(19) United States
(12) Patent Application Publication
(10) Pub. No.: US 2025/0230155 A1
(43) Pub. Date: Jul. 17, 2025

(54) 7,8-DIHYDRO-5H-1,6-NAPHTHYRIDINE DERIVATIVES AS POSITIVE ALLOSTERIC MODULATORS OF THE MUSCARINIC ACETYLCHOLINE RECEPTOR M4 FOR TREATING NEUROLOGICAL AND PSYCHIATRIC DISORDERS

(71) Applicant: Vanderbilt University, Nashville, TN (US)

(72) Inventors: Craig W. Lindsley, Brentwood, TN (US); P. Jeffrey Conn, Nashville, TN (US); Darren W. Engers, Brentwood, TN (US); Kayla J. Temple, Spring Hill, TN (US); Julie L. Engers, Brentwood, TN (US); Madeline F. Long, Nashville, TN (US); Logan A. Baker, Thompson's Station, TN (US); Alison R. Gregro, Mount Juliet, TN (US); Charlotte Park, Nashville, TN (US); Paul K. Spearing, Chapel Hill, TN (US)

(21) Appl. No.: 18/700,110

(22) PCT Filed: Oct. 14, 2022
(86) PCT No.: PCT/US2022/046760

§ 371 Date: Apr. 10, 2024

Related U.S. Application Data
(60) Provisional application No. 63/300,826, filed on Jan. 19, 2022.
(60) Provisional application No. 63/255,899, filed on Oct. 14, 2021.

(51) Int. Cl. C07D 471/04; A61K 31/444; A61K 31/501; A61K 31/506; A61K 31/517; A61K 31/519; A61K 31/5377; A61K 31/538; A61K 31/553; C07B 59/00; C07D 519/00

(52) U.S. Cl. CPC C07D 471/04; A61K 31/444; A61K 31/501; A61K 31/506; A61K 31/517; A61K 31/519; A61K 31/5377; A61K 31/538; A61K 31/553; C07B 59/002; C07D 519/00

(57) ABSTRACT

The present invention relates to 7,8-dihydro-5H-1,6-naphthyridine derivatives of formula (I) The present compounds are positive allosteric modulators of the muscarinic acetylcholine receptor M4 (mAChR M4) for use in treating neurological and psychiatric disorders associated with muscarinic acetylcholine receptor dysfunction. An exemplary compound is e.g. compound 1 Data on the activity of exemplary compounds in an mAChR M4 cell-based assay is provided.

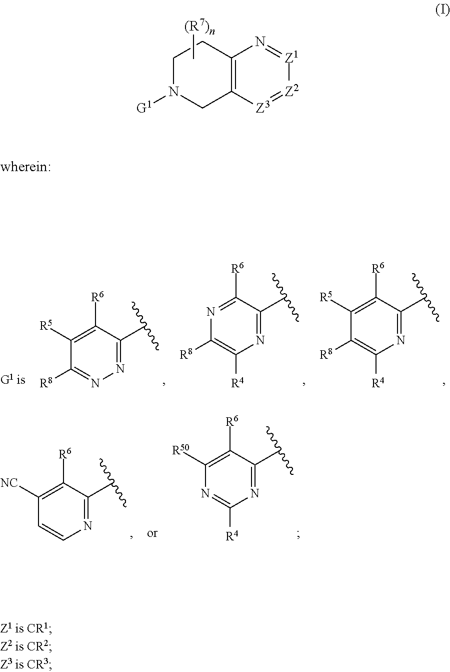

| | | | |
|---|---|---|---|
| 1 | 5-methyl-6- (3-pyrrolidin- 1-1-7,8- dihydro-5H-1,6- naphtbyridin- 6-yl)pyridine- 3-carbonitrile | 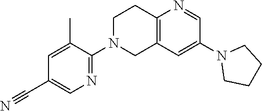 [SMILES: Cc1cc(C#N)cnc1N1CCc2ncc(N3CCCC3)cc2C1] | 320.2 |

### RELATED APPLICATIONS

This application claims priority to U.S. Provisional Application No. 63/255,899, filed Oct. 14, 2021 and U.S. Provisional Application No. 63/300,826, filed Jan. 19, 2022, each of which is hereby incorporated by reference in its entirety.

### TECHNICAL FIELD

The present disclosure relates to compounds, compositions, and methods for treating neurological and psychiatric disorders associated with muscarinic acetylcholine receptor dysfunction.

### BACKGROUND

Cholinergic neurotransmission involves the activation of nicotinic acetylcholine receptors (nAChRs) or the muscarinic acetylcholine receptors (mAChRs) by the binding of the endogenous orthosteric agonist acetylcholine (ACh). Conditions associated with cognitive impairment, such as Alzheimer's disease, are accompanied by a reduction of acetylcholine content in the brain. This is believed to be the result of degeneration of cholinergic neurons of the basal forebrain, which widely innervate multiple areas of the brain, including the association cortices and hippocampus, which are critically involved in higher processes. Clinical data supports that cholinergic hypofunction contributes to the cognitive deficits of patients suffering from schizophrenia. Efforts to increase acetylcholine levels have focused on increasing levels of choline, the precursor for acetylcholine synthesis, and on blocking acetylcholinesterase (AChE), the enzyme that metabolizes acetylcholine. As a result, acetylcholinesterase (AChE) inhibitors, which inhibit the hydrolysis of ACh, have been approved in the United States for use in the palliative, but not disease-modifying, treatment of the cognitive deficits in AD patients.

Attempts to augment central cholinergic function through the administration of choline or phosphatidylcholine have not been successful. AChE inhibitors have shown therapeutic efficacy, but have been found to have frequent cholinergic side effects due to peripheral acetylcholine stimulation, including abdominal cramps, nausea, vomiting, and diarrhea. These gastrointestinal side effects have been observed in about a third of the patients treated. In addition, some AChE inhibitors, such as tacrine, have also been found to cause significant hepatotoxicity with elevated liver transaminases observed in about 30% of patients. The adverse effects of AChE inhibitors have severely limited their clinical utility. An alternative approach to pharmacologically target cholinergic hypofunction is the activation of mAChRs, which are widely expressed throughout the body.

The mAChRs are members of the family A G protein-coupled receptors (GPCRs) and include five subtypes, designated M₁-M₅. The M₁, M₃ and M₅ subtypes mainly couple to G_{q} and activate phospholipase C, whereas the M₂ and M₄ subtypes mainly couple to G_{i/o} and associated effector systems. These five distinct mAChR subtypes have been identified in the mammalian central nervous system where they are prevalent and differentially expressed. M₁-M₅ have varying roles in cognitive, sensory, motor and autonomic functions. Thus, without wishing to be bound by a particular theory, it is believed that selective agonists of mAChR subtypes that regulate processes involved in cognitive function could prove to be superior therapeutics for treatment of psychosis, schizophrenia and related disorders. The muscarinic M₄ receptor has been shown to have a major role in cognitive processing and is believed to have a major role in the pathophysiology of psychotic disorders, including schizophrenia.

Evidence suggests that the most prominent adverse effects of AChE inhibitors and other cholinergic agents are mediated by activation of peripheral M₂ and M₃ mAChRs and include bradycardia, GI distress, excessive salivation, and sweating. In contrast, M₄ has been viewed as the most likely subtype for mediating the effects of muscarinic acetylcholine receptor dysfunction in psychotic disorders, including schizophrenia, cognition disorders, and neuropathic pain. Because of this, considerable effort has been focused on developing selective M₄ agonists for treatment of these disorders. Unfortunately, these efforts have been largely unsuccessful because of an inability to develop compounds that are highly selective for the mAChR M₄. Because of this, mAChR agonists that have been tested in clinical studies induce a range of adverse effects by activation of peripheral mAChRs. To fully understand the physiological roles of individual mAChR subtypes and to further explore the therapeutic utility of mAChR ligands in psychosis, including schizophrenia, cognition disorders and other disorders, it can be important to develop compounds that are highly selective activators of mAChR M₄ and other individual mAChR subtypes.

Previous attempts to develop agonists that are highly selective for individual mAChR subtypes have failed because of the high conservation of the orthosteric ACh binding site. To circumvent problems associated with targeting the highly conserved orthosteric ACh binding site, it is believed that developing compounds that act at allosteric sites on mAChRs that are removed from the orthosteric site and are less highly conserved. This approach is proving to be highly successful in developing selective ligands for multiple GPCR subtypes. In the case of mAChRs, a major goal has been to develop allosteric ligands that selectively increase activity of mAChR M₄ or other mAChR subtypes. Allosteric activators can include allosteric agonists, that act at a site removed from the orthosteric site to directly activate the receptor in the absence of ACh as well as positive allosteric modulators (PAMs), which do not activate the receptor directly but potentiate activation of the receptor by the endogenous orthosteric agonist ACh. Also, it is possible for a single molecule to have both allosteric potentiator and allosteric agonist activity.

More recently, muscarinic agonists including xanomeline have been shown to be active in animal models with similar profiles to known antipsychotic drugs, but without causing catalepsy (Bymaster et al., Eur. J. Pharmacol. 1998, 356, 109, Bymaster et al., Life Sci. 1999, 64, 527; Shannon et al., J. Pharmacol. Exp. Ther. 1999, 290, 901; Shannon et al., Schizophrenia Res. 2000, 42, 249). Further, xanomeline was shown to reduce psychotic behavioral symptoms such as delusions, suspiciousness, vocal outbursts, and hallucinations in Alzheimer's disease patients (Bodick et al., Arch. Neurol. 1997, 54, 465), however treatment induced side effects, e.g., gastrointestinal effects, have severely limited the clinical utility of this compound.

Despite advances in muscarinic acetylcholine receptor research, there is still a scarcity of compounds that are potent, efficacious, and selective activators of the M₄ mAChR and also effective in the treatment of neurological and psychiatric disorders associated with cholinergic activity and diseases in which the muscarinic M₄ receptor is involved.

### SUMMARY

In one aspect, disclosed are compounds of formula (I), or a pharmaceutically acceptable salt thereof

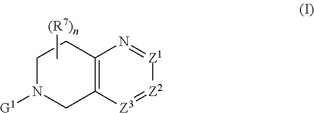

wherein:
G¹ is

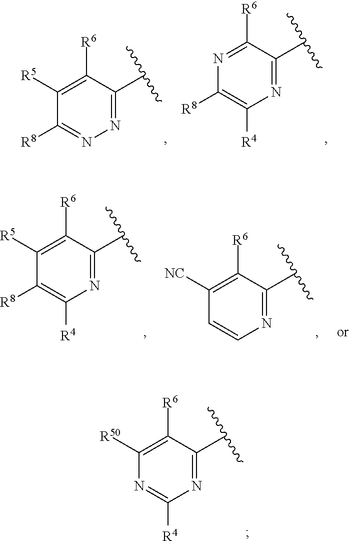

Z is N or CR¹;
Z² is CR²;
Z3 is N or CR³
R¹ is hydrogen, halogen, cyano, C₁₋₆alkyl, C₁₋₆haloalkyl, —OR^{H}—NR^{b}R^{c}, —SR^{b}, —OC(O)R^{b}, —NR^{b}C(O)R^{c}, —NR^{b}SO₂R^{a}, —C(O)OR^{b}, —C(O)NR^{b}R^{c}, —SO₂NR^{b}R^{c}, —C(O)R^{b}, —S(O)R^{a}, —SO₂R^{a}, G², or —C₁₋₃alkylene-G²;
R² and R³ are each independently hydrogen, halogen, cyano, NO₂, C₁₋₆alkyl, C₁₋₆haloalkyl, —OR^{b}, —NR^{b}R^{c}, —SR^{b}, —OC(O)R^{b}, —NR^{b}C(O)R^{c}, —NR^{b}SO₂R^{a}, —C(O)OR^{b}, —C(O)N^{b}R^{c}R, —SO₂NR^{b}R^{c}, —C(O)R^{b}, —S(O)R^{a}, —SO₂R^{a}, —C₁₋₆alkylene-OH, —C₁₋₆fluoroalkylene-OH, G², or —C₁₋₃alkylene-G²;
R^{a}, at each occurrence, is independently C₁₋₆alkyl, C₁₋₆haloalkyl, G², or —C₁₋₃alkylene-G²;
R^{b} and R^{c}, at each occurrence, are independently hydrogen, C₁₋₆alkyl, C₁₋₆haloalkyl, G², —C₁₋₃alkylene-G², —C₂₋₄alkylene-O—C₁₋₄alkyl, or —C₂₋₄alkylene-O-G²;
R^{H} is hydrogen, C₁₋₆alkyl, C₁₋₆haloalkyl, G^{2H}, or —C₁₋₃alkylene-G^{2H}.
G², at each occurrence, is independently a 6- to 12-membered aryl, a 5- to 12-membered heteroaryl, a 4- to 12-membered heterocyclyl, or a 3- to 12-membered carbocyclyl, wherein G² is optionally substituted with 1-5 substituents independently selected from the group consisting of halogen, cyano, C₁₋₆alkyl, C₁₋₆haloalkyl, oxo, —OR^{x}, —N(R^{x})₂, —C₁₋₆alkylene-OR^{x}, —C₁₋₆alkylene-N(R^{x})₂, G^{2a}, and —C₁₋₃alkylene-G^{2a};
G^{2H} is a 6- to 12-membered aryl, a 5- to 12-membered heteroaryl, a 4- to 8-membered heterocyclyl containing 1-2 oxygen atoms, or a 3- to 12-membered carbocyclyl, wherein G^{2H} is optionally substituted with 1-5 substituents independently selected from the group consisting of halogen, cyano, C₁₋₄alkyl, C₁₋₄haloalkyl, oxo, —OR^{x}, —N(R^{x})₂, G^{2a}, and —C₁₋₃alkylene-G^{2a};
R^{x}, at each occurrence, is independently hydrogen, C₁₋₄alkyl, C₁₋₄haloalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄haloalkyl;
R⁴ is hydrogen, halogen, cyano, C₁₋₆alkyl, C₁₋₆haloalkyl, —C₁₋₆alkylene-OR^{4a}, —OR^{4a}, —O—C₂₋₆alkylene-OR^{4a}, N(R^{4a})₂, —N(R^{4a})—C₂₋₆alkylene-OR^{4a}, G³, —O—G³, —N(R^{4a})-G³, —C₁₋₃alkylene-G³, —O—C₁₋₃alkylene-G³, or —N(R^{4a})—C₁₋₃alkylene-G³;
R^{4a}, at each occurrence, is independently hydrogen, C₁₋₆alkyl, or C₁₋₆haloalkyl;
R⁵ and R⁶ are each independently hydrogen, halogen, cyano, C₁₋₆alkyl, C₁₋₆haloalkyl, OH, —OC₁₋₆alkyl, —OC₁₋₆haloalkyl, G³, —O—G³, —C₁₋₃alkylene-G³, or —O—C₁₋₃alkylene-G³;
R⁵⁰ is hydrogen, halogen, cyano, C₁₋₆alkyl, C₁₋₆haloalkyl, OH, —OC₁₋₆alkyl, —OC₁₋₆haloalkyl, G³⁰, —O—G³, —C(O)—N(R^{50a})₂—C₁₋₃alkylene-G³, or —O—C₁₋₃alkylene-G³;
R^{50a}, at each occurrence, is independently hydrogen, C₁₋₄alkyl, G³, or —C₁₋₃alkylene-G³, or two R^{50a}, together with the nitrogen to which they attach, form a 4- to 8-membered heterocyclyl, the heterocyclyl optionally containing a second heteroatom that is O, N, or S and being optionally substituted with 1-4 C₁₋₄alkyl;
wherein, alternatively, R⁵⁰ and R⁶, together with the atom to which each attaches, form a 5- to 7-membered non-aromatic carbocyclic or heterocyclic ring, the heterocyclic ring containing one heteroatom that is O, N, or S and the carbocyclic or heterocyclic ring being optionally substituted with 1-4 C₁₋₄alkyl;
G³, at each occurrence, is independently a 6- to 12-membered aryl, a 5- to 12-membered heteroaryl, a 4- to 12-membered heterocyclyl, or a 3- to 12-membered carbocyclyl, wherein G³ is optionally substituted with 1-5 substituents independently selected from the group consisting of halogen, cyano, C₁₋₄alkyl, C₁₋₄haloalkyl, oxo, OH, —OC₁₋₄alkyl, —OC₁₋₄haloalkyl, G^{3a}, and —C₁₋₃alkylene-G^{3a};
G³⁰ is a 6- to 12-membered aryl, a 5- to 12-membered heteroaryl other than indazol-3-yl, a 4- to 12-membered heterocyclyl, or a 3- to 12-membered carbocyclyl, wherein G³⁰ is optionally substituted with 1-5 substituents independently selected from the group consisting of halogen, cyano, C₁₋₄alkyl, C₁₋₄haloalkyl, oxo, OH, —OC₁₋₄alkyl, —OC₁₋₄haloalkyl, G^{3a}, and —C₁₋₃alkylene-G^{3a};
R⁷, at each occurrence, is independently halogen, C₁₋₄alkyl, C₁₋₄haloalkyl, C₃₋₇cycloalkyl, OH, —OC₁₋₄alkyl, or —OC₁₋₄haloalkyl, wherein the cycloalkyl is optionally substituted with 1-4 substituents independently selected from the group consisting of halogen, C₁₋₄alkyl, and C₁₋₄haloalkyl;
R⁸ is halogen, cyano, C₁₋₆alkyl, C₁₋₆haloalkyl, —OR^{8b}, —NR^{8b}R^{8c}, —SR^{8b}, —OC(O)R^{8b}, —NR^{8b}C(O)R^{8c}, —NR^{8b}SO₂R^{8a}, —C(O)OR^{8b}, —C(O)NR^{8b}R^{8c}, —SO₂NR^{8b}R^{8c}—C(O)R^{8b}, —S(O)R^{8a}, —SO₂R^{8a}, G⁴, or —C₁₋₃alkylene-G⁴;
R^{8a}, at each occurrence, is independently C₁₋₆alkyl, C₁₋₆haloalkyl, G⁴, or —C₁₋₃alkylene-G⁴;
R^{8b} and R^{8c}, at each occurrence, are independently hydrogen, C₁₋₆alkyl, C₁₋₆haloalkyl, —C₁₋₆alkylene-OH, G⁴, or —C₁₋₃alkylene-G⁴; wherein, alternatively, when R⁸ is —C(O)NR^{8b}R^{8c}, and R⁴ is —OR^{4a}, R^{8c} and R^{4a}, together with the atom to which each attaches, form an oxazepin-5-one ring;
G⁴, at each occurrence, is independently a 6- to 12-membered aryl, a 5- to 12-membered heteroaryl, a 4- to 12-membered heterocyclyl, or a 3- to 12-membered carbocyclyl optionally fused to a 6-membered arene or heteroarene that contains 1-2 nitrogen atoms, wherein G⁴ is optionally substituted with 1-5 substituents independently selected from the group consisting of halogen, cyano, C₁₋₄alkyl, C₁₋₄haloalkyl, —C₁₋₆alkylene-OH, oxo, —OR⁸⁰, —N(R⁸⁰)₂, —NR⁸⁰C(O)R⁸⁰, —NR⁸⁰SO₂R⁸⁰, —C(O)OR⁸⁰, —C(O)N(R⁸⁰)₂, —SO₂R⁸⁰, G^{4a}, and —C₁₋₃alkylene-G^{4a}.
R⁸⁰, at each occurrence, is independently hydrogen, C₁₋₄alkyl, C₁₋₄haloalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄haloalkyl;
G^{2a} G^{3a}, and G^{4a}, at each occurrence, are independently a phenyl, a 5- to 6-membered heteroaryl, a 4- to 8-membered heterocyclyl, or a 3- to 8-membered carbocyclyl, wherein G^{2a}, G^{3a}, and G^{4a}, at each occurrence, are independently optionally substituted with 1-5 substituents independently selected from the group consisting of halogen, cyano, C₁₋₄alkyl, C₁₋₄haloalkyl, —C₁₋₆alkylene-OH, oxo, OH, —OC₁₋₄alkyl, —OC₁₋₄haloalkyl, C₃₋₄cycloalkyl, and —C₁₋₃alkylene-C₃₋₄cycloalkyl;
and
n is 0, 1, 2, 3, or 4;
provided that the compound is not
6-[3-chloro-5-(trifluoromethyl)-2-pyridinyl]-5,6,7,8-tetrahydro-3-methoxy-pyrido[4,3-c]pyridazine;
6-(7,8-dihydro-1,6-naphthyridin-6(5H)-yl)-N-[2-(1-methyl-2-piperidinyl)ethyl]-3-pyridinecarboxamide;
6-(7,8-dihydro-1,6-naphthyridin-6(5H)-yl)-N-[(3-propyl-5-isoxazolyl)methyl]-3-pyridinecarboxamide;
6-(6-methyl-2-(pyridin-3-yl)pyrimidin-4-yl)-5,6,7,8-tetrahydro-1,6-naphthyridine;
4-[7,8-dihydro-3-(1-pyrrolidinyl)pyrido[4,3-c]pyridazin-6(5H)-yl]-2-pyrimidinecarbonitrile;
4-(7,8-dihydro-3-methoxypyrido[4,3-c]pyridazin-6(5H)-yl)-2-pyrimidinecarbonitrile;
6-(2,6-dimethyl-4-pyrimidinyl)-5,6,7,8-tetrahydro-1,6-naphthyridin-4-amine;
5,6,7,8-tetrahydro-6-[2-(1-piperidinyl)-4-pyrimidinyl]-pyrido[4,3-c]pyridazin-3(2H)-one; or
5,6,7,8-tetrahydro-6-[6-(1-piperidinyl)-4-pyrimidinyl]-pyrido[4,3-c]pyridazin-3(2H)-one, or a salt thereof.

In another aspect, disclosed are compounds of formula (I), or a pharmaceutically acceptable salt thereof

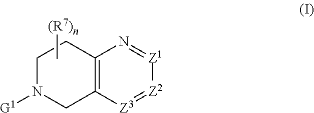

wherein:
G¹ is

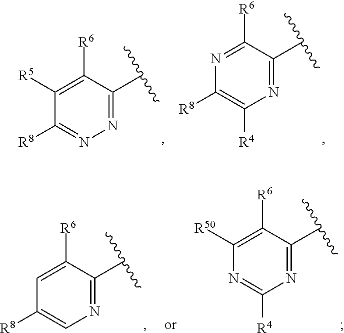

Z¹ is N or CR¹;
Z² is CR²;
Z³ is N or CR³;
R¹ is hydrogen, halogen, cyano, C₁₋₆alkyl, C₁₋₆haloalkyl, —OR^{H}, —R^{c}, —SR^{b}, —OC(O)R^{b}, —NR^{b}C(O)R, —NR^{b}SO₂R^{a}, —C(O)OR^{b}, —C(O)NR^{b}R^{c}, —SO₂NR^{b}R^{c}, —C(O)R^{b}, —S(O)R^{a}—SO₂R^{a}, G², or —C₁₋₃alkylene-G²;
R² and R³ are each independently hydrogen, halogen, cyano, C₁₋₆alkyl, C₁₋₆haloalkyl, —OR^{b}, —NR^{b}R^{c}, —SR^{b}, —OC(O)R^{b}, —NR^{b}C(O)R, —NR^{b}SO₂R^{a}, —C(O)OR^{b}, —C(O)R^{b}R^{c}, —SO₂NR^{b}R^{c}, —C(O)R^{b}, —S(O)R^{a}, —SO₂R^{a}, G², or —C₁₋₃alkylene-G²;
R^{a}, at each occurrence, is independently C₁₋₆alkyl, C₁₋₆haloalkyl, G², or —C₁₋₃alkylene-G²;
R^{b} and R^{c}, at each occurrence, are independently hydrogen, C₁₋₆alkyl, C₁₋₆haloalkyl, G², or —C₁₋₃alkylene-G²;
R^{H} is hydrogen, C₁₋₆alkyl, C₁₋₆haloalkyl, G^{2H}, or —C₁₋₃alkylene-G^{2H};
G², at each occurrence, is independently a 6- to 12-membered aryl, a 5- to 12-membered heteroaryl, a 4- to 12-membered heterocyclyl, or a 3- to 12-membered carbocyclyl, wherein G² is optionally substituted with 1-5 substituents independently selected from the group consisting of halogen, cyano, C₁₋₄alkyl, C₁₋₄haloalkyl, oxo, —OR^{x}, —N(R^{x})₂, G^{2a}, and —C₁₋₃alkylene-G^{2a};
G^{2H} is a 6- to 12-membered aryl, a 5- to 12-membered heteroaryl, a 4- to 8-membered heterocyclyl containing 1-2 oxygen atoms, or a 3- to 12-membered carbocyclyl, wherein G^{2H} is optionally substituted with 1-5 substituents independently selected from the group consisting of halogen, cyano, C₁₋₄alkyl, C₁₋₄haloalkyl, oxo, —OR^{x}, —N(R^{x})₂, G^{2a}, and —C₁₋₃alkylene-G^{2a};
R^{x}, at each occurrence, is independently hydrogen, C₁₋₄alkyl, C₁₋₄haloalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄haloalkyl;
R⁴, R⁵ and R⁶ are each independently hydrogen, halogen, cyano, C₁₋₆alkyl, C₁₋₆haloalkyl, OH, —OC₁₋₆alkyl, —OC₁₋₆haloalkyl, G³, —O—G³, —C₁₋₃alkylene-G³, or —O—C₁₋₃alkylene-G³;
R⁵⁰ is hydrogen, halogen, cyano, C₁₋₆alkyl, C₁₋₆haloalkyl, OH, —OC₁₋₆alkyl, —OC₁₋₆haloalkyl, G³⁰, —O—G³, —C₁₋₃alkylene-G³, or —O—C₁₋₃alkylene-G³;
G³, at each occurrence, is independently a 6- to 12-membered aryl, a 5- to 12-membered heteroaryl, a 4- to 12-membered heterocyclyl, or a 3- to 12-membered carbocyclyl, wherein G³ is optionally substituted with 1-5 substituents independently selected from the group consisting of halogen, cyano, C₁₋₄alkyl, C₁₋₄haloalkyl, oxo, OH, —OC₁₋₄alkyl, —OC₁₋₄haloalkyl, G^{3a}, and —C₁₋₃alkylene-G^{3a};
G³⁰ is a 6- to 12-membered aryl, a 5- to 12-membered heteroaryl other than indazol-3-yl, a 4- to 12-membered heterocyclyl, or a 3- to 12-membered carbocyclyl, wherein G³⁰ is optionally substituted with 1-5 substituents independently selected from the group consisting of halogen, cyano, C₁₋₄alkyl, C₁₋₄haloalkyl, oxo, OH, —OC₁₋₄alkyl, —OC₁₋₄haloalkyl, G^{3a}, and —C₁₋₃alkylene-G^{3a};
R⁷, at each occurrence, is independently halogen, C₁₋₄alkyl, C₁₋₄haloalkyl, C₃₋₇cycloalkyl, OH, —OC₁₋₄alkyl, or —OC₁₋₄haloalkyl, wherein the cycloalkyl is optionally substituted with 1-4 substituents independently selected from the group consisting of halogen, C₁₋₄alkyl, and C₁₋₄haloalkyl;
R⁸ is halogen, cyano, C₁₋₆alkyl, C₁₋₆haloalkyl, —OR^{8b}, —NR^{8b}R^{8c}, —SR^{8b}, —OC(O)R^{8b}, —NR^{8b}C(O)R^{8c}—NR^{8b}SO₂R^{8a}, —C(O)OR^{8b}, —C(O)NR^{8b}R^{8c}, —SO₂NR^{8b}R^{8c}, —C(O)R^{8b}, —S(O)R^{8a}, —SO₂R^{8a}, G⁴, or —C₁₋₃alkylene-G⁴;
R^{8a}, at each occurrence, is independently C₁₋₆alkyl, C₁₋₆haloalkyl, G⁴, or —C₁₋₃alkylene-G⁴;
R^{8b} and R^{8c}, at each occurrence, are independently hydrogen, C₁₋₆alkyl, C₁₋₆haloalkyl, G⁴, or —C₁₋₃alkylene-G⁴; G⁴, at each occurrence, is independently a 6- to 12-membered aryl, a 5- to 12-membered heteroaryl, a 4- to 12-membered heterocyclyl, or a 3- to 12-membered carbocyclyl, wherein
G⁴ is optionally substituted with 1-5 substituents independently selected from the group consisting of halogen, cyano, C₁₋₄alkyl, C₁₋₄haloalkyl, —N(R⁸⁰)₂, —NR⁸⁰ C(O)R⁸⁰, —NR⁸⁰ SO₂R⁸⁰, —C(O)OR⁸⁰, —C(O)N(R⁸⁰)₂, —SO₂R⁸⁰, G^{4a}, and —C₁₋₃alkylene-G⁴a;
R⁸⁰, at each occurrence, is independently hydrogen, C₁₋₄alkyl, C₁₋₄haloalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄haloalkyl;
G^{2a}, G^{3a}, and G^{4a}, at each occurrence, are independently a phenyl, a 5- to 6-membered heteroaryl, a 4- to 8-membered heterocyclyl, or a 3- to 8-membered carbocyclyl, wherein G^{2a}, G^{3a}, and G^{4a}, at each occurrence, are independently optionally substituted with 1-5 substituents independently selected from the group consisting of halogen, cyano, C₁₋₄alkyl, C₁₋₄haloalkyl, oxo, OH, —OC₁₋₄alkyl, and —OC₁₋₄haloalkyl;
and
n is 0, 1, 2, 3, or 4;
provided that the compound is not
6-[3-chloro-5-(trifluoromethyl)-2-pyridinyl]-5,6,7,8-tetrahydro-3-methoxy-pyrido[4,3-c]pyridazine;
6-(7,8-dihydro-1,6-naphthyridin-6(5H)-yl)-N-[2-(1-methyl-2-piperidinyl)ethyl]-3-pyridinecarboxamide;
6-(7,8-dihydro-1,6-naphthyridin-6(5H)-yl)-N-[(3-propyl-5-isoxazolyl)methyl]-3-pyridinecarboxamide;
6-(6-methyl-2-(pyridin-3-yl)pyrimidin-4-yl)-5, 6,7,8-tetrahydro-1,6-naphthyridine;
4-[7,8-dihydro-3-(1-pyrrolidinyl)pyrido[4,3-c]pyridazin-6(5H)-yl]-2-pyrimidinecarbonitrile;
4-(7,8-dihydro-3-methoxypyrido[4,3-c]pyridazin-6(5H)-yl)-2-pyrimidinecarbonitrile;
6-(2,6-dimethyl-4-pyrimidinyl)-5,6,7,8-tetrahydro-1,6-naphthyridin-4-amine;
5,6,7,8-tetrahydro-6-[2-(1-piperidinyl)-4-pyrimidinyl]-pyrido[4,3-c]pyridazin-3(2H)-one; or
5,6,7,8-tetrahydro-6-[6-(1-piperidinyl)-4-pyrimidinyl]-pyrido[4,3-c]pyridazin-3(2H)-one, or a salt thereof.

In another aspect, the invention provides a pharmaceutical composition comprising a compound of formula (I), or a pharmaceutically acceptable salt thereof, and a pharmaceutically acceptable carrier.

Another aspect provides a method of treating a neurological and/or psychiatric disorder associated with muscarinic acetylcholine receptor dysfunction in a mammal, comprising administering to the mammal a therapeutically effective amount of the compound of formula (I), or pharmaceutically acceptable salt or composition thereof.

Another aspect provides a compound of formula (I), or a pharmaceutically acceptable salt or composition thereof, for use in the treatment of a neurological and/or psychiatric disorder associated with muscarinic acetylcholine receptor dysfunction in a mammal.

Another aspect provides use of a compound of formula (I), or a pharmaceutically acceptable salt or composition thereof, for the preparation of a medicament for the treatment of a neurological and/or psychiatric disorder associated with muscarinic acetylcholine receptor dysfunction in a mammal.

In another aspect, the invention provides kits comprising a compound of formula (I), or a pharmaceutically acceptable salt or composition thereof, and instructions for use.

### DETAILED DESCRIPTION

Disclosed herein are positive allosteric modulators (i.e. potentiators) of the muscarinic acetylcholine receptor M₄ (mAChR M₄), methods of making same, pharmaceutical compositions comprising same, and methods of treating neurological and psychiatric disorders associated with muscarinic acetylcholine receptor dysfunction using same. The compounds include naphthyridine-substituted pyridazine compounds.

The human muscarinic acetylcholine receptor M₄ (mAChR M₄) is a protein of 479 amino acids encoded by the CHRM₄ gene. The molecular weight of the unglycosylated protein is about 54 kDa and it is a transmembrane GPCR. As described above, the mAChR M₄ is a member of the GPCR Class A family, or the rhodopsin-like GPCRs, which are characterized by structural features similar to rhodopsin such as seven transmembrane segments. The muscarinic acetylcholine receptors have the N-terminus oriented to the extracellular face of the membrane and the C-terminus located on the cytoplasmic face.

Previous attempts to develop agonists that are highly selective for individual mAChR subtypes have failed because of the high conservation of the orthosteric ACh binding site. To circumvent problems associated with targeting the highly conserved orthosteric ACh binding site, it is believed that developing compounds that act at allosteric sites on mAChRs that are removed from the orthosteric site and are less highly-conserved. Without wishing to be bound by a particular theory, the disclosed compounds and products of the disclosed methods are believed to bind to an allosteric site distinct from the orthosteric binding site.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain saturated hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched saturated chain hydrocarbon, for example, of 1 to 6 carbon atoms. Representative examples of alkylene include, but are not limited to, —CH₂—, —CH₂CH₂—, —CH₂CH₂CH₂—, —CH₂CH(CH₃)CH₂—, —CH₂CH₂CH₂CH₂—, —CH₂CH(CH₃)CH₂CH₂—, and —CH₂CH₂CH₂CH₂CH₂—.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NR^{x}R^{y}, wherein R^{x} and R^{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NR^{x}—, wherein R^{x} may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkoxy,” as used herein, means at least one fluoroalkyl group, as defined herein, is appended to the parent molecular moiety through an oxygen atom. Representative examples of fluoroalkoxy include, but are not limited to, difluoromethoxy, trifluoromethoxy and 2,2,2-trifluoroethoxy.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl is an 8- to 12-membered ring system and includes a fused bicyclic heteroaromatic ring system (i.e., 107π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 107π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl(thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

Where heterocyclic and heteroaromatic ring systems are defined to “contain” or as “containing” specified heteroatoms (e.g., 1-3 heteroatoms independently selected from the group consisting of O, N, and S), any ring atoms of the heterocyclic and heteroaromatic ring systems that are not one of the specified heteroatoms are carbon atoms.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “sulfonamide,” as used herein, means —S(O)₂NR^{z}— or —NR^{z}S(O)—, wherein R^{z} may be hydrogen, alkyl, cycloalkyl, aryl, heteroaryl, heterocycle, alkenyl, or heteroalkyl.

The term “substituents” refers to a group “substituted” on a group such as an alkyl, alkenyl, alkynyl, aryl, heteroaryl, cycloalkyl, heteroalkyl, or heterocycle group, at any atom of that group. Any atom can be substituted.

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl. In some embodiments, a group is optionally substituted. In some embodiments, a group is optionally substituted with 1, 2, 3, 4, or 5 substituents. In some embodiments, an aryl, heteroaryl, cycloalkyl, or heterocycle is optionally substituted with 1, 2, 3, 4, or 5 substituents. In some embodiments, an aryl, heteroaryl, cycloalkyl, or heterocycle may be independently unsubstituted or substituted with 1, 2, or 3 substituents.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “allosteric site” as used herein refers to a ligand binding site that is topographically distinct from the orthosteric binding site.

The term “modulator” as used herein refers to a molecular entity (e.g., but not limited to, a ligand and a disclosed compound) that modulates the activity of the target receptor protein.

The term “ligand” as used herein refers to a natural or synthetic molecular entity that is capable of associating or binding to a receptor to form a complex and mediate, prevent or modify a biological effect. Thus, the term “ligand” encompasses allosteric modulators, inhibitors, activators, agonists, antagonists, natural substrates and analogs of natural substrates.

The terms “natural ligand” and “endogenous ligand” as used herein are used interchangeably, and refer to a naturally occurring ligand, found in nature, which binds to a receptor.

The term “orthosteric site” as used herein refers to the primary binding site on a receptor that is recognized by the endogenous ligand or agonist for that receptor. For example, the orthosteric site in the mAChR M₄ receptor is the site that acetylcholine binds.

The term “mAChR M₄ receptor positive allosteric modulator” as used herein refers to any exogenously administered compound or agent that directly or indirectly augments the activity of the mAChR M₄ receptor in the presence or in the absence of acetylcholine, or another agonist, in an animal, in particular a mammal, for example a human. For example, a mAChR M₄ receptor positive allosteric modulator can increase the activity of the mAChR M₄ receptor in a cell in the presence of extracellular acetylcholine. The cell can be Chinese hamster ovary (CHO-K1) cells transfected with human mAChR M₄. The cell can be Chinese hamster ovary (CHO-K1) cells transfected with rat mAChR M₄ receptor. The cell can be Chinese hamster ovary (CHO-K1) cells transfected with a mammalian mAChR M₄. The term “mAChR M₄ receptor positive allosteric modulator” includes a compound that is a “mAChR M₄ receptor allosteric potentiator” or a “mAChR M₄ receptor allosteric agonist,” as well as a compound that has mixed activity comprising pharmacology of both an “mAChR M₄ receptor allosteric potentiator” and an “mAChR M₄ receptor allosteric agonist.” The term “mAChR M₄ receptor positive allosteric modulator also includes a compound that is a “mAChR M₄ receptor allosteric enhancer.”

The term “mAChR M₄ receptor allosteric potentiator” as used herein refers to any exogenously administered compound or agent that directly or indirectly augments the response produced by the endogenous ligand (such as acetylcholine) when the endogenous ligand binds to the orthosteric site of the mAChR M₄ receptor in an animal, in particular a mammal, for example a human. The mAChR M₄ receptor allosteric potentiator binds to a site other than the orthosteric site, that is, an allosteric site, and positively augments the response of the receptor to an agonist or the endogenous ligand. In some embodiments, an allosteric potentiator does not induce desensitization of the receptor, activity of a compound as an mAChR M₄ receptor allosteric potentiator provides advantages over the use of a pure mAChR M₄ receptor orthosteric agonist. Such advantages can include, for example, increased safety margin, higher tolerability, diminished potential for abuse, and reduced toxicity.

The term “mAChR M₄ receptor allosteric enhancer” as used herein refers to any exogenously administered compound or agent that directly or indirectly augments the response produced by the endogenous ligand (such as acetylcholine) in an animal, in particular a mammal, for example a human. In some embodiments, the allosteric enhancer increases the affinity of the natural ligand or agonist for the orthosteric site. In some embodiments, an allosteric enhancer increases the agonist efficacy. The mAChR M₄ receptor allosteric enhancer binds to a site other than the orthosteric site, that is, an allosteric site, and positively augments the response of the receptor to an agonist or the endogenous ligand. An allosteric enhancer has no effect on the receptor by itself and requires the presence of an agonist or the natural ligand to realize a receptor effect.

The term “mAChR M₄ receptor allosteric agonist” as used herein refers to any exogenously administered compound or agent that directly activates the activity of the mAChR M₄ receptor in the absence of the endogenous ligand (such as acetylcholine) in an animal, in particular a mammal, for example a human. The mAChR M₄ receptor allosteric agonist binds to a site that is distinct from the orthosteric acetylcholine site of the mAChR M₄ receptor. Because it does not require the presence of the endogenous ligand, activity of a compound as an mAChR M₄ receptor allosteric agonist provides advantages if cholinergic tone at a given synapse is low.

The term “mAChR M₄ receptor neutral allosteric ligand” as used herein refers to any exogenously administered compound or agent that binds to an allosteric site without affecting the binding or function of agonists or the natural ligand at the orthosteric site in an animal, in particular a mammal, for example a human. However, a neutral allosteric ligand can block the action of other allosteric modulators that act via the same site.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Compounds

In one aspect, disclosed is a compound of formula (I), wherein Z¹, Z², Z³, G¹, R⁷, and n are as defined herein. Embodiments of formula (I) include the following descriptions of Z¹, Z², Z³, G¹, R⁷, and n, and any combinations thereof.

In some embodiments, G¹ is

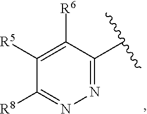

wherein R⁵, R⁶, and R⁸ are as defined herein. Accordingly, in some embodiments the compounds of formula (I) have formula (I-a), wherein Z¹, Z², Z³, R⁵, R⁶, R⁷, R⁸, and n are as defined herein (e.g., R⁸ is cyano, —C(O)OR^{8b}, —C(O)R^{8b}, or —C(O)NR^{8b}R^{8c}).

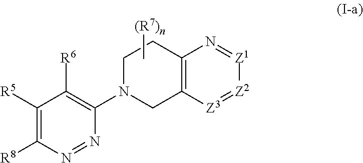

An exemplary embodiment within formula (I-a) is illustrated by formula (I-aa), wherein R², R⁵, R⁶, and R⁸ are as defined herein (e.g., R⁸ is cyano, —C(O)OR^{8b}, —C(O)R^{1b}, or —C(O)NR^{8b}R^{8c}).

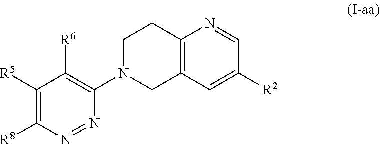

In further embodiments of formulas (I), (I-a) or (I-aa), R⁵ and R⁶ are independently C₁₋₄alkyl. In still further embodiments, Rand R⁶ are CH₃.

In some embodiments, G¹ is

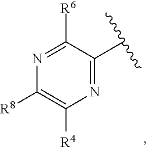

wherein R⁴, R⁶, and R⁸ are as defined herein. Accordingly, in some embodiments the compounds of formula (I) have formula (I-b), wherein Z¹, Z², Z³, R⁴, R⁶, R⁷, R⁸, and n are as defined herein.

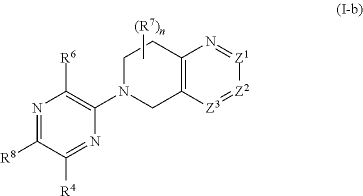

An exemplary embodiment within formula (I-b) is illustrated by formula (I-ba), wherein R², R⁴, R⁶, and R⁸ are as defined herein.

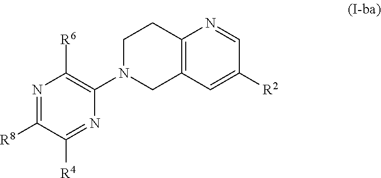

In further embodiments of formulas (I), (I-b) or (I-ba), R⁴ is hydrogen.

In some embodiments, G¹ is

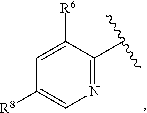

wherein R⁶ is as defined herein. Accordingly, in some embodiments the compounds of formula (I) have formula (I-c), wherein Z¹, Z², Z³, R⁶, R⁷, R⁸, and n are as defined herein (e.g., R⁸ is cyano, —C(O)OR^{8b}, —C(O)R^{8b}, or —C(O)N^{8b}R^{8c})

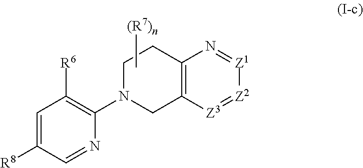

An exemplary embodiment within formula (I-c) is illustrated by formula (I-ca), wherein R², R⁶, and R⁸ are as defined herein (e.g., R⁸ is cyano, —C(O)OR^{8b}, —C(O)R^{8b}, or —C(O)NR^{b}R^{8c}).

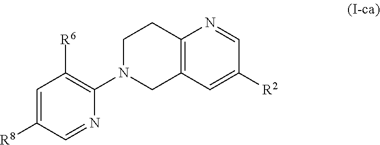

In further embodiments of formulas (I), (I-c) or (1-ca), R⁶ is C₁.4alkyl. In still further embodiments, R⁶ is CH₃.

In some embodiments, G¹ is

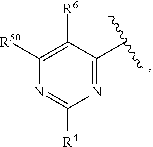

wherein R⁴, R⁵⁰, and R⁶ are as defined herein. Accordingly, in some embodiments the compounds of formula (I) have formula (I-d), wherein Z¹, Z², Z³, R⁴, R⁶, R⁷, R⁵⁰, and n are as defined herein. In some embodiments, wherein R⁵⁰ is G³⁰, G³⁰ is not an indazole. In some embodiments wherein R⁵⁰ is G³⁰, G³⁰ is a 6-to 12-membered aryl, a 5- to 6-membered heteroaryl, a 4- to 12-membered heterocyclyl, or a 3-to 12-membered carbocyclyl, wherein G³⁰ is optionally substituted as defined herein.

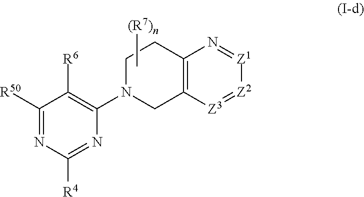

An exemplary embodiment within formula (I-d) is illustrated by formula (I-da), wherein R², R⁴, R⁶, and R⁵⁰ are as defined herein.

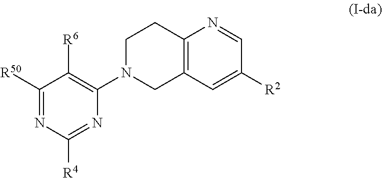

In further embodiments of formulas (I), (I-d) or (I-da), R⁴, R⁵⁰, and R⁶ are independently hydrogen, halogen (e.g., chloro, fluoro), C₁₋₄alkyl (e.g., methyl), —OC₁₋₄alkyl (e.g., —OCH₃), or C₃₋₈cycloalkyl (e.g., cyclopropyl). In still further embodiments, R⁴ is C₁₋₄alkyl (e.g., methyl) or C₃₋₆cycloalkyl (e.g., cyclopropyl); R⁵⁰ is halogen or —OC₁₋₄alkyl (e.g., —OCH₃); and R⁶ is C₁₋₄alkyl (e.g., methyl).

In the embodiments herein are further embodiments wherein Z¹ is CR¹; and Z³ is CR³ and R¹ and R³ are as defined herein. In further embodiments, R¹ and R³ are hydrogen.

In some embodiments, R² is hydrogen, C₁₋₆alkyl, C₁₋₆haloalkyl, —OR^{b}, —NR^{b}R^{c}, G², or —C₁₋₃alkylene-G², wherein R^{b}, R^{c}, and G² are as defined herein.

In some embodiments, G² is a) a 5- to 6-membered or 9- to 10-membered heteroaryl containing 1-3 heteroatoms independently selected from the group consisting of O, N, and S; b) a phenyl; c) a phenyl fused to a 5- to 7-membered heterocycle containing 1-2 oxygen atoms; or d) a 4- to 8-membered monocyclic heterocyclyl containing 1-2 heteroatoms independently selected from the group consisting of O, N, and S; wherein G² is optionally substituted with 1-4 substituents independently selected from the group consisting of halogen (chloro, fluoro), cyano, C₁₋₄alkyl (e.g., methyl, n-propyl, isopropyl), C₁₋₄haloalkyl (e.g., CF³), —OR^{x} (e.g., —OC₁₋₄alkyl such as —OCH₃, —OC₁₋₄haloalkyl such as —OCF₃), —N(R^{x})₂ (e.g., —N(C₁₋₄alkyl)₂ such as —N(CH₃)₂), G^{2a}, and —CH₂-G^{2a}; and G^{2a} is C₃₋₆cycloalkyl (e.g., cyclopropyl, cyclohexyl) or phenyl and optionally substituted with 1-4 substituents independently selected from the group consisting of halogen, C₁₋₄alkyl, and C₁₋₄haloalkyl, wherein R^{x} is as defined herein. In further embodiments, the phenyl fused to a 5- to 7-membered heterocycle containing 1-2 oxygen atoms is not optionally substituted. In further embodiments, the 5- to 6-membered heteroaryl, 9- to 10-membered heteraryl, and phenyl are optionally substituted as defined above. In further embodiments, the 4- to 8-membered heterocyclyl is optionally substituted with 1-4 substituents independently selected from the group consisting of halogen and C₁₋₄alkyl.

In some embodiments, G² is pyrazolyl (e.g., pyrazol-3-yl, pyrazol-4-yl), thiazolyl (e.g., thiazol-5-yl), isoxazolyl (e.g., isoxazol-4-yl), pyridinyl (e.g., pyridin-2-yl, pyridin-3-yl, pyridin-4-yl), pyridazinyl (e.g., pyridazin-4-yl), pyrimidinyl (e.g., pyrimidin-4-yl, pyrimidin-5-yl), benzimidazolyl (e.g., benzimidazol-5-yl), imidazo[1,2-a]pyridinyl (e.g., imidazo[1,2-a]pyridine-6-yl), indazolyl (e.g., indazol-5-yl), phenyl, 1,4-benzodioxin-6-yl, tetrahydrofuranyl (e.g., tetrahydrofuran-2-yl), 1,4-dioxanyl (e.g., 1,4-dioxan-2-yl), azetidinyl (e.g., azetidin-1-yl), pyrrolidinyl (e.g., pyrrolidin-1-yl), or piperidinyl (e.g., piperidin-1-yl), wherein G² is optionally substituted as described herein.

In some embodiments, R² is G², wherein G² is as defined herein. In further embodiments, G² is a 5- to 6-membered or 9- to 10-membered heteroaryl containing 1-3 heteroatoms independently selected from the group consisting of O, N, and S (e.g., pyrazolyl, isoxazolyl, thiazolyl, pyridinyl, pyridazinyl, pyrimidinyl, benzimidazolyl, imidazo[1,2-a]pyridinyl); or phenyl, wherein G² is optionally substituted as defined herein (e.g., 1-4 substituents independently selected from the group consisting of halogen (chloro, fluoro), cyano, C₁₋₄alkyl (e.g., methyl, n-propyl, isopropyl), C₁₋₄haloalkyl (e.g., CF³), —OR^{x} (e.g., —OC₁₋₄alkyl such as —OCH₃, —OC₁₋₄haloalkyl such as —OCF₃), —N(R^{x})₂ (e.g., —N(C₁₋₄alkyl)₂ such as —N(CH₃)₂), G^{2a}, and —CH₂-G^{2a}; wherein G^{2a} is C₃₋₆cycloalkyl (e.g., cyclopropyl, cyclohexyl) or phenyl and optionally substituted with 1-4 substituents independently selected from the group consisting of halogen, C₁₋₄alkyl, and C₁₋₄haloalkyl, wherein R^{x} is as defined herein (e.g., methyl)). In further embodiments, G² is a 4- to 8-membered monocyclic heterocyclyl containing 1-2 heteroatoms independently selected from the group consisting of O, N, and S (e.g., azetidinyl, pyrrolidinyl, piperidinyl), wherein G² is optionally substituted as described herein (e.g., 1-4 substituents independently selected from the group consisting of halogen, C₁₋₄alkyl, and C₁₋₄haloalkyl. In some embodiments, G² is an optionally substituted 4- to 8-membered heterocyclyl attached through a ring nitrogen atom (e.g., piperidin-1-yl).

In some embodiments, R² is —NR^{b}R^{c}, wherein R^{b} and R^{c} are as defined herein. In some embodiments, R² is —NR^{b}R^{c}; one of R^{b} and R^{c} is G² or —C₁₋₃alkylene-G² and the other is as defined herein. In some embodiments, R² is —NR^{b}R^{c}; R^{b} is G² or —C₁₋₃alkylene-G²; and R^{c} is hydrogen, C₁₋₆alkyl, C₁₋₆haloalkyl, C₃₋₆cycloalkyl, or —C₁₋₃alkylene-C₃₋₆cycloalkyl, wherein G² is as defined herein. In further embodiments, R^{c} is hydrogen. In further embodiments, G² is a) a 5- to 6-membered or 9- to 10-membered heteroaryl heteroaryl containing 1-3 heteroatoms independently selected from the group consisting of O, N, and S (e.g., pyrazolyl, pyridinyl, pyrimidinyl); b) a phenyl; or c) a 4- to 8-membered monocyclic heterocyclyl containing 1-2 heteroatoms independently selected from the group consisting of O, N, and S (e.g., tetrahydrofuranyl); wherein G² (e.g., phenyl, pyrazolyl, pyridinyl, pyrimidinyl) is optionally substituted with 1-4 substituents independently selected from the group consisting of halogen, cyano, C₁₋₄alkyl, C₁₋₄haloalkyl, —OR^{x}, —N(R^{x})₂, G^{2a}, and —CH₂-G^{2a}; and G^{2a} is C₃₋₆cycloalkyl optionally substituted with 1-4 substituents independently selected from the group consisting of halogen, C₁₋₄alkyl, and C₁₋₄haloalkyl, wherein R^{x} is as defined herein (e.g., methyl). In further embodiments, G² is phenyl, pyrazolyl, pyridinyl, pyrimidinyl, tetrahydrofuranyl, or 1,4-dioxanyl, wherein the phenyl, pyrazolyl, pyridinyl, and pyrimidinyl are optionally substituted as described herein. In some embodiments, R² is —R^{b}R^{c}; R^{b} is G²; and R^{c} and G² are as defined herein. In further embodiments, G² is a) an optionally substituted 5- to 6-membered or 9- to 10-membered heteroaryl containing 1-3 heteroatoms independently selected from the group consisting of O, N, and S; or b) optionally substituted phenyl. In still further embodiments, G² is a phenyl, pyrazolyl, pyridinyl, or pyrimidinyl, each optionally substituted with 1-4 substituents independently selected from the group consisting of halogen (e.g., fluoro, chloro), cyano, C₁₋₄alkyl (e.g., methyl), C₁₋₄haloalkyl (e.g., CF³), —OR^{x}, (e.g., —OC₁₋₄alkyl such as —OCH₃, —OC₁₋₄haloalkyl such as —OCF₃), —N(R^{x})₂ (e.g., —N(C₁₋₄alkyl)₂ such as —NMe₂), G^{2a}, and —CH₂—G^{2a}. In other embodiments, R² is —NR^{b}R^{c}; R^{b} is —CH₂—G²; and R^{c} and G² are as defined herein. In further embodiments, G² is a) an optionally substituted 5- to 6-membered or 9- to 10-membered heteroaryl containing 1-3 heteroatoms independently selected from the group consisting of O, N, and S; or b) optionally substituted phenyl. In still further embodiments, G² is a phenyl or pyridinyl optionally substituted with 1-4 substituents independently selected from the group consisting of halogen (e.g., fluoro, chloro), cyano, C₁₋₄alkyl (e.g., methyl), C₁₋₄haloalkyl (e.g., CF³), —OR^{x} (e.g., —OC₁₋₄alkyl such as —OCH₃, —OC₁₋₄haloalkyl such as —OCF₃), —N(R^{x})₂, G^{2a}, and —CH₂—G^{2a}. In other embodiments, R² is —NR^{b}R^{c}; R^{b} is —CH₂—G²; R^{c} is as defined herein; and G² is a 4- to 8-membered heterocyclyl containing 1-2 oxygen atoms (e.g., tetrahydrofuranyl, 1,4-dioxanyl).

In some embodiments, R² is —C₁₋₃alkylene-G², wherein G² is as defined herein (e.g., optionally substituted phenyl).

In some embodiments, R² is —OR^{b}; and R^{b} is G². In further embodiments, G² is a) a 5- to 6-membered or 9- to 10-membered heteroaryl containing 1-3 heteroatoms independently selected from the group consisting of O, N, and S (e.g., pyridinyl, indazolyl); b) a phenyl; or c) a phenyl fused to a 5- to 7-membered heterocycle containing 1-2 oxygen atoms (e.g., 1,4-benzodioxin-6-yl), wherein G² is optionally substituted with 1-4 substituents independently selected from the group consisting of halogen (e.g. fluoro), cyano, C₁₋₄alkyl (e.g., methyl), C₁₋₄haloalkyl, —OR^{x} (e.g., —OC₁₋₄alkyl such as —OCH₃), —N(R^{x})₂, G^{2a}, and —CH₂—G^{2a}; and G^{2a} is C₃₋₆ optionally substituted with 1-4 substituents independently selected from the group consisting of halogen, C₁₋₄alkyl, and C₁₋₄haloalkyl, wherein R^{x} is as defined herein (e.g., methyl). In still further embodiments, G² is a) a 6-membered or 9- to 10-membered heteroaryl containing 1-3 nitrogen atoms (e.g., pyridinyl, indazolyl); or b) a phenyl, wherein the heteroaryl and phenyl are optionally substituted as defined above; or G² is c) a phenyl fused to a 5- to 7-membered heterocycle containing 1-2 oxygen atoms (no optional substituent).

In some embodiments, R² is hydrogen, halogen, C₁₋₆alkyl, C₁₋₆haloalkyl,

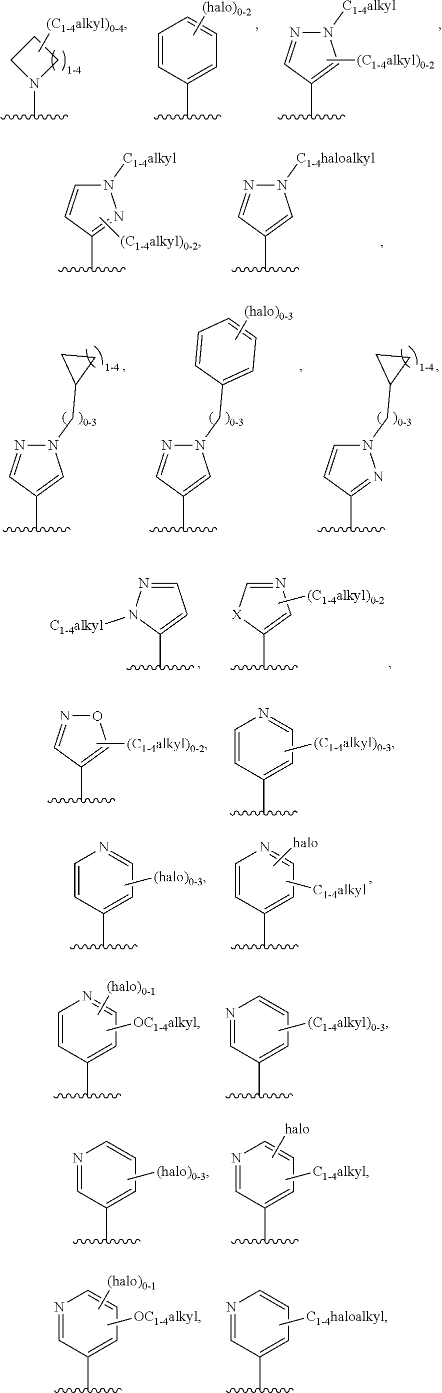

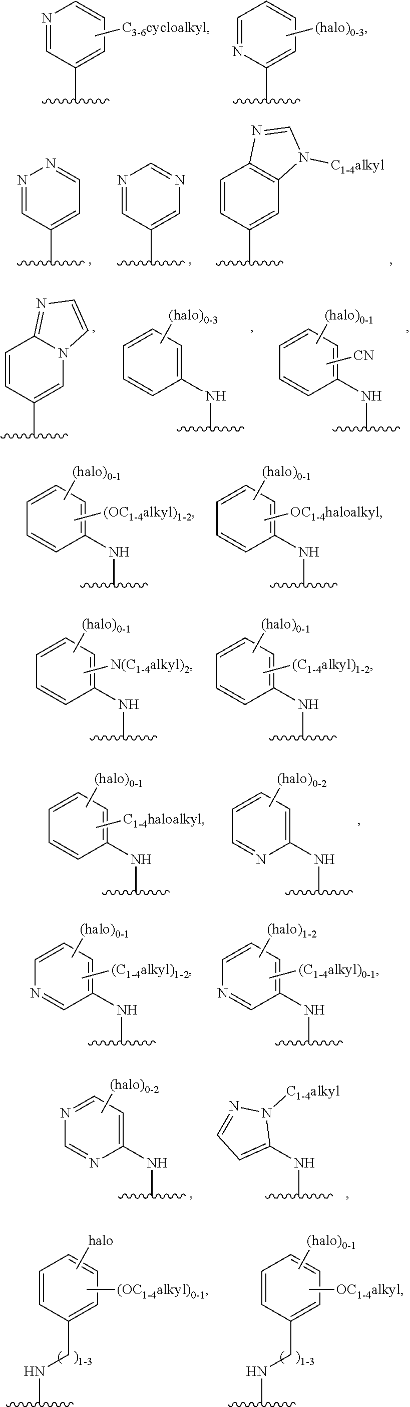

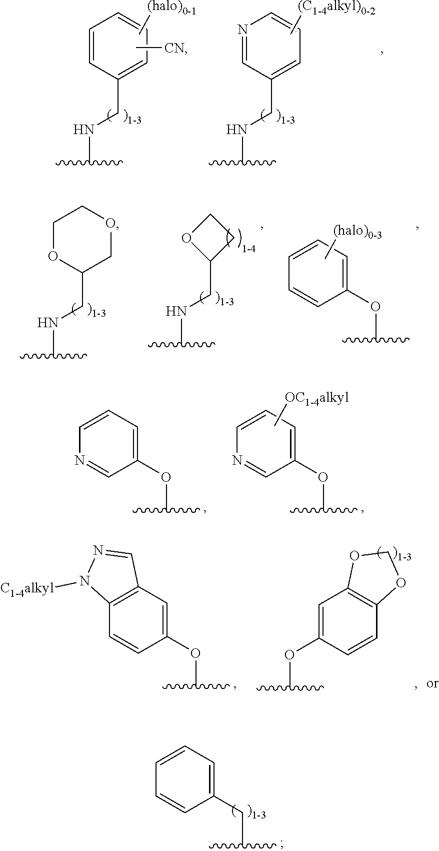

and X is O, N, or S. When X is N, hydrogen substitution is present on the nitrogen in the absence of alkyl substitution.

In the embodiments herein are further embodiments wherein R⁸ is cyano, —C(O)OR^{8b}, —C(O)R^{8b}, or —C(O)NR^{8b}R^{8c}, wherein Z¹, Z², Z³, R⁴, R⁵, R⁶, R⁷, n, R^{8b} and R^{8c} are as defined herein. In embodiments wherein R^{8b} or R^{8c} includes a G⁴ group, are embodiments where G⁴ may be a) a 5- to 6-membered or 9- to 10-membered heteroaryl containing 1-3 heteroatoms independently selected from the group consisting of 0, N, and S; b) phenyl; c) a phenyl fused to a 5- to 7-membered heterocycle containing 1-2 heteroatoms independently selected from the group consisting of 0, N, and S; d) a 4- to 10-membered heterocyclyl containing 1-3 heteroatoms independently selected from the group consisting of 0, N, and S; or e) a C₃₋₈cycloalkyl, wherein G⁴ is optionally substituted as defined herein (e.g., 1-4 substituents independently selected from the group consisting of halogen (e.g., fluoro, chloro, bromo), cyano, C₁₋₄alkyl (e.g., methyl), C₁₋₄haloalkyl (e.g., CF³), OH, oxo, —OC₁₋₄alkyl (e.g., —OCH₃), —OC₁₋₄haloalkyl, —C(O)OC₁₋₄alkyl (e.g., —C(O)OCH₃), —C(O)NHC₁₋₄alkyl (e.g., —C(O)NHCH₃), —C(O)N(C₁₋₄alkyl)₂, —SO₂C₁₋₄alkyl (e.g., —SO₂CH₃), G⁴a (e.g., pyrazolyl, pyrazol-1-yl, imidazolyl, imidazol-1-yl), and —C₁₋₃alkylene-G^{4a}). For example, a G^{4a} in an optional substituent may be a 5- to 6-membered heteroaryl containing 1-3 heteroatoms independently selected from the group consisting of O, N, and S (e.g., pyrazolyl).

In some embodiments, R⁸ is cyano and Z¹, Z², Z³, R⁴, R⁵, R⁶, R⁷, and n are as defined herein.

In some embodiments, R⁸ is —C(O)OR^{8b}, and Z¹, Z², Z³, R⁴, R⁵, R⁶, R⁷, n, and R^{gb} are as defined herein. For example, R^{8b} may be C₁₋₆alkyl.

In some embodiments, R⁸ is —C(O)R^{8b}, and Z¹, Z², Z³, R⁴, R⁵, R⁶, R⁷, n, and R^{gb} are as defined herein. In further embodiments, R^{8b} is G⁴. In still further embodiments, the G⁴ is a 4- to 10-membered heterocyclyl containing 1-3 heteroatoms independently selected from the group consisting of O, N, and S, wherein G⁴ is optionally substituted as defined herein. For example G⁴ may be a 5- to 7-membered nitrogen containing heterocyclyl attached through a ring nitrogen and optionally fused to a phenyl (e.g., 1,2,3,4-tetrahydroisoquinolin-2-yl, indolin-1-yl) or to a 6-membered heteroaryl containing 1-2 nitrogen atoms (e.g., 5,6,7,8-tetrahydro-1,6-naphthyridin-6-yl).

In some embodiments, R⁸ is —C(O)NR^{8b}R^{8c}, wherein Z¹, Z², Z³, R⁴, R⁵, R⁶, R⁷, n, R^{8b} and R^{8c} are as defined herein. In further embodiments, R^{8b} is C₁₋₆alkyl, G⁴, or —C₁₋₃alkylene-G⁴ (e.g., —CH₂—G⁴). In still further embodiments, G⁴ is a) a 5- to 6-membered or 9- to 10-membered heteroaryl containing 1-3 heteroatoms independently selected from the group consisting of O, N, and S (e.g., pyridinyl, pyrimidinyl, pyrrolyl, thiazolyl, isoxazolyl, pyrazolyl, benzothiazolyl, benzothiophenyl, benzofuranyl, benzotriazolyl, quinolinyl, quinoxalinyl, pyrazolo[1,5-a]pyridinyl, imidazo[1,2-a]pyridinyl, [1,2,4]triazolo[1,5-a]pyridinyl, 6,7-dihydro-5H-cyclopenta[b]pyridin-3-yl); b) phenyl; c) a phenyl fused to a 5- to 7-membered heterocycle containing 1-2 heteroatoms independently selected from the group consisting of O, N, and S (e.g., 1,3-dihydroisobenzofuran-5-yl, indolin-5-yl, 2,3-dihydro-1H-benzo[d]imidazol-5-yl, 2,3-dihydrothiophen-5-yl); d) a 4- to 10-membered heterocyclyl containing 1-3 heteroatoms independently selected from the group consisting of O, N, and S (e.g., tetrahydropyranyl); or e) a C₃₋₈cycloalkyl (e.g., cyclopropyl, cyclopentyl, cyclohexyl, bicyclo[1.1.1]pentanyl), wherein G⁴ is optionally substituted as defined herein. In some embodiments, G⁴ is optionally substituted with 1-4 substituents independently selected from the group consisting of halogen (e.g., fluoro, chloro, bromo), cyano, C₁₋₄alkyl (e.g., methyl), C₁₋₄haloalkyl (e.g., CF³), OH, oxo, —OC₁₋₄alkyl (e.g., —OCH₃), —OC₁₋₄haloalkyl, —C(O)OC₁₋₄alkyl (e.g., —C(O)OCH₃), —C(O)NHC₁₋₄alkyl (e.g., —C(O)NHCH₃), —C(O)N(C₁₋₄alkyl)₂, —SO₂C₁₋₄alkyl (e.g., —SO₂CH₃), G⁴a (e.g., pyrazolyl, pyrazol-1-yl, imidazolyl, imidazol-1-yl), and —C₁₋₃alkylene-G^{4a}. In further embodiments wherein R⁸ is —C(O)NR^{8b}R^{8c}, R^{8c} is hydrogen.

In the embodiments herein wherein R⁸ is —C(O)OR^{8b}, —C(O)R^{1b}, or —C(O)NR^{8b}R^{8c} are still further embodiments wherein G¹ is

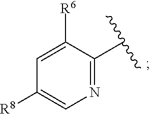

and R⁶, —C(O)OR^{8b}, —C(O)R^{8b} and —C(O)NR^{8b}R^{8c} are as defined herein.

In the embodiments herein wherein R⁸ is —C(O)OR^{8b}, —C(O)R^{1b}, or —C(O)NR^{8b}R^{8c} are still further embodiments wherein G¹ is

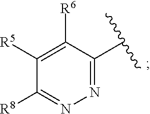

and R⁵, R⁶, R⁸, —C(O)OR^{8b}, —C(O)R^{8b}, and —C(O)NR^{8c} are as defined herein.

In the embodiments herein are further embodiments wherein n is 0.

Representative compounds of formula (I) include, but are not limited to:
5-methyl-6-(3-pyrrolidin-1-yl-7,8-dihydro-5H-1,6-naphthyridin-6-yl)pyridine-3-carbonitrile;
6-[3-(4-fluoroanilino)-7,8-dihydro-5H-1,6-naphthyridin-6-yl]-5-methyl-pyridine-3-carbonitrile;
6-[3-(4-fluorophenyl)-7,8-dihydro-5H-1,6-naphthyridin-6-yl]-5-methyl-pyridine-3-carbonitrile;
6-[3-[(4-fluorophenyl)methylamino]-7,8-dihydro-5H-1,6-naphthyridin-6-yl]-5-methyl-pyridine-3-carbonitrile;
6-[3-[(3-fluoro-4-methoxy-phenyl)methylamino]-7,8-dihydro-5H-1,6-naphthyridin-6-yl]-5-methyl-pyridine-3-carbonitrile;
5-methyl-6-[3-(2-pyridylamino)-7,8-dihydro-5H-1,6-naphthyridin-6-yl]pyridine-3-carbonitrile;
6-[3-(2-fluoroanilino)-7,8-dihydro-5H-1,6-naphthyridin-6-yl]-5-methyl-pyridine-3-carbonitrile;
6-[3-(1,4-dioxan-2-ylmethylamino)-7,8-dihydro-5H-1,6-naphthyridin-6-yl]-5-methyl-pyridine-3-carbonitrile;
5-methyl-6-[3-(tetrahydrofuran-2-ylmethylamino)-7,8-dihydro-5H-1,6-naphthyridin-6-yl]pyridine-3-carbonitrile;
6-[3-[1-(cyclopropylmethyl)pyrazol-3-yl]-7,8-dihydro-5H-1,6-naphthyridin-6-yl]-4,5-dimethyl-pyridazine-3-carbonitrile;
4,5-dimethyl-6-[3-(1-methylpyrazol-4-yl)-7,8-dihydro-5H-1,6-naphthyridin-6-yl]pyridazine-3-carbonitrile;
4,5-dimethyl-6-[3-(2-methyl-4-pyridyl)-7,8-dihydro-5H-1,6-naphthyridin-6-yl]pyridazine-3-carbonitrile;
6-[3-(1-cyclopropylpyrazol-4-yl)-7,8-dihydro-5H-1,6-naphthyridin-6-yl]-4,5-dimethyl-pyridazine-3-carbonitrile;
4,5-dimethyl-6-[3-(2-methylpyrazol-3-yl)-7,8-dihydro-5H-1,6-naphthyridin-6-yl]pyridazine-3-carbonitrile;
6-[3-(1-cyclohexylpyrazol-3-yl)-7,8-dihydro-5H-1,6-naphthyridin-6-yl]-4,5-dimethyl-pyridazine-3-carbonitrile;
4,5-dimethyl-6-[3-(1-propylpyrazol-4-yl)-7,8-dihydro-5H-1,6-naphthyridin-6-yl]pyridazine-3-carbonitrile;
4,5-dimethyl-6-[3-(3-methylbenzimidazol-5-yl)-7,8-dihydro-5H-1,6-naphthyridin-6-yl]pyridazine-3-carbonitrile;
6-[3-(2-fluorophenyl)-7,8-dihydro-5H-1,6-naphthyridin-6-yl]-4,5-dimethyl-pyridazine-3-carbonitrile;
6-(3-imidazo[1,2-a]pyridin-6-yl-7,8-dihydro-5H-1,6-naphthyridin-6-yl)-4,5-dimethyl-pyridazine-3-carbonitrile;
4,5-dimethyl-6-[3-(2-phenylethyl)-7,8-dihydro-5H-1,6-naphthyridin-6-yl]pyridazine-3-carbonitrile;
6-[3-(3-fluorophenyl)-7,8-dihydro-5H-1,6-naphthyridin-6-yl]-4,5-dimethyl-pyridazine-3-carbonitrile;
4,5-dimethyl-6-(3-pyrrolidin-1-yl-7,8-dihydro-5H-1,6-naphthyridin-6-yl)pyridazine-3-carbonitrile;
4,5-dimethyl-6-[3-(1-piperidyl)-7,8-dihydro-5H-1,6-naphthyridin-6-yl]pyridazine-3-carbonitrile;
6-[3-[(3-fluorophenyl)methylamino]-7,8-dihydro-5H-1,6-naphthyridin-6-yl]-4,5-dimethyl-pyridazine-3-carbonitrile;
6-[3-[(4-methoxyphenyl)methylamino]-7,8-dihydro-5H-1,6-naphthyridin-6-yl]-4,5-dimethyl-pyridazine-3-carbonitrile;
6-[3-[(4-cyanophenyl)methylamino]-7,8-dihydro-5H-1,6-naphthyridin-6-yl]-4,5-dimethyl-pyridazine-3-carbonitrile;
6-[3-[(3-methoxyphenyl)methylamino]-7,8-dihydro-5H-1,6-naphthyridin-6-yl]-4,5-dimethyl-pyridazine-3-carbonitrile;
6-[3-(3,3-dimethylazetidin-1-yl)-7,8-dihydro-5H-1,6-naphthyridin-6-yl]-4,5-dimethyl-pyridazine-3-carbonitrile;
4,5-dimethyl-6-[3-[(6-methyl-3-pyridyl)methylamino]-7,8-dihydro-5H-1,6-naphthyridin-6-yl]pyridazine-3-carbonitrile;
6-[3-(2-fluoroanilino)-7,8-dihydro-5H-1,6-naphthyridin-6-yl]-4,5-dimethyl-pyridazine-3-carbonitrile;
4,5-dimethyl-6-(3-thiazol-5-yl-7,8-dihydro-5H-1,6-naphthyridin-6-yl)pyridazine-3-carbonitrile;
6-[3-(5-cyano-2-fluoro-anilino)-7,8-dihydro-5H-1,6-naphthyridin-6-yl]-4,5-dimethyl-pyridazine-3-carbonitrile;
6-[3-(2-chloro-6-fluoro-anilino)-7,8-dihydro-5H-1,6-naphthyridin-6-yl]-4,5-dimethyl-pyridazine-3-carbonitrile;
6-[3-(2-fluoro-3-methoxy-anilino)-7,8-dihydro-5H-1,6-naphthyridin-6-yl]-4,5-dimethyl-pyridazine-3-carbonitrile;
6-[3-[2-fluoro-3-(trifluoromethyl)anilino]-7,8-dihydro-5H-1,6-naphthyridin-6-yl]-4,5-dimethyl-pyridazine-3-carbonitrile;
6-[3-(3-chloro-2-fluoro-anilino)-7,8-dihydro-5H-1,6-naphthyridin-6-yl]-4,5-dimethyl-pyridazine-3-carbonitrile;
6-[3-(2-fluoro-5-methyl-anilino)-7,8-dihydro-5H-1,6-naphthyridin-6-yl]-4,5-dimethyl-pyridazine-3-carbonitrile;
6-[3-(2,4-difluoroanilino)-7,8-dihydro-5H-1,6-naphthyridin-6-yl]-4,5-dimethyl-pyridazine-3-carbonitrile;
6-[3-(2-chloroanilino)-7,8-dihydro-5H-1,6-naphthyridin-6-yl]-4,5-dimethyl-pyridazine-3-carbonitrile;
6-[3-[2-(dimethylamino)anilino]-7,8-dihydro-5H-1,6-naphthyridin-6-yl]-4,5-dimethyl-pyridazine-3-carbonitrile;
6-[3-(2-cyanoanilino)-7,8-dihydro-5H-1,6-naphthyridin-6-yl]-4,5-dimethyl-pyridazine-3-carbonitrile;
4,5-dimethyl-6-[3-(2-methylanilino)-7,8-dihydro-5H-1,6-naphthyridin-6-yl]pyridazine-3-carbonitrile;
4,5-dimethyl-6-[3-[2-(trifluoromethyl)anilino]-7,8-dihydro-5H-1,6-naphthyridin-6-yl]pyridazine-3-carbonitrile;
4,5-dimethyl-6-[3-[2-(trifluoromethoxy)anilino]-7,8-dihydro-5H-1,6-naphthyridin-6-yl]pyridazine-3-carbonitrile;
6-(7,8-dihydro-5H-1,6-naphthyridin-6-yl)-5-methyl-pyridine-3-carbonitrile;
6-[3-(4-chloro-2-fluoro-anilino)-7,8-dihydro-5H-1,6-naphthyridin-6-yl]-4,5-dimethyl-pyridazine-3-carbonitrile;
4,5-dimethyl-6-[3-(3-pyridyl)-7,8-dihydro-5H-1,6-naphthyridin-6-yl]pyridazine-3-carbonitrile;
4,5-dimethyl-6-(3-pyridazin-4-yl-7,8-dihydro-5H-1,6-naphthyridin-6-yl)pyridazine-3-carbonitrile;
4,5-dimethyl-6-(3-pyrimidin-5-yl-7,8-dihydro-5H-1,6-naphthyridin-6-yl)pyridazine-3-carbonitrile;
4,5-dimethyl-6-[3-[2-(trifluoromethyl)-3-pyridyl]-7,8-dihydro-5H-1,6-naphthyridin-6-yl]pyridazine-3-carbonitrile;
6-[3-(6-chloro-5-fluoro-3-pyridyl)-7,8-dihydro-5H-1,6-naphthyridin-6-yl]-4,5-dimethyl-pyridazine-3-carbonitrile;
6-[3-(2-chloro-5-methoxy-3-pyridyl)-7,8-dihydro-5H-1,6-naphthyridin-6-yl]-4,5-dimethyl-pyridazine-3-carbonitrile;
6-[3-(2-chloro-5-fluoro-3-pyridyl)-7,8-dihydro-5H-1,6-naphthyridin-6-yl]-4,5-dimethyl-pyridazine-3-carbonitrile;
6-[3-(2-fluoro-3-pyridyl)-7,8-dihydro-5H-1,6-naphthyridin-6-yl]-4,5-dimethyl-pyridazine-3-carbonitrile;
4,5-dimethyl-6-[3-(4-methyl-3-pyridyl)-7,8-dihydro-5H-1,6-naphthyridin-6-yl]pyridazine-3-carbonitrile;
6-[3-(6-chloro-3-pyridyl)-7,8-dihydro-5H-1,6-naphthyridin-6-yl]-4,5-dimethyl-pyridazine-3-carbonitrile;
6-(7,8-dihydro-5H-1,6-naphthyridin-6-yl)-N,5-dimethyl-pyridine-3-carboxamide;
6-(7,8-dihydro-5H-1,6-naphthyridin-6-yl)-N-isopropyl-5-methyl-pyridine-3-carboxamide;
6-(7,8-dihydro-5H-1,6-naphthyridin-6-yl)-5-methyl-N-(2-pyridylmethyl)pyridine-3-carboxamide;
6-(7,8-dihydro-5H-1,6-naphthyridin-6-yl)-5-methyl-N-(3-pyridylmethyl)pyridine-3-carboxamide;
6-(7,8-dihydro-5H-1,6-naphthyridin-6-yl)-5-methyl-N-(4-pyridylmethyl)pyridine-3-carboxamide;
6-(7,8-dihydro-5H-1,6-naphthyridin-6-yl)-5-methyl-N-(thiazol-5-ylmethyl)pyridine-3-carboxamide;
6-(7,8-dihydro-5H-1,6-naphthyridin-6-yl)-5-methyl-N-[(2-methylpyrazol-3-yl)methyl]pyridine-3-carboxamide;
N-cyclopropyl-6-(7,8-dihydro-5H-1,6-naphthyridin-6-yl)-5-methyl-pyridine-3-carboxamide;
N-cyclopentyl-6-(7,8-dihydro-5H-1,6-naphthyridin-6-yl)-5-methyl-pyridine-3-carboxamide;
N-cyclohexyl-6-(7,8-dihydro-5H-1,6-naphthyridin-6-yl)-5-methyl-pyridine-3-carboxamide;
6-(7,8-dihydro-5H-1,6-naphthyridin-6-yl)-5-methyl-N-[(4-pyrazol-1-ylphenyl)methyl]pyridine-3-carboxamide;
6-(7,8-dihydro-5H-1,6-naphthyridin-6-yl)-5-methyl-N-(6-quinolylmethyl)pyridine-3-carboxamide;
6-(7,8-dihydro-5H-1,6-naphthyridin-6-yl)-5-methyl-N-(pyrazolo[1,5-a]pyridin-3-ylmethyl)pyridine-3-carboxamide;
N-benzyl-6-(7,8-dihydro-5H-1,6-naphthyridin-6-yl)-5-methyl-pyridine-3-carboxamide;
methyl 6-(7,8-dihydro-5H-1,6-naphthyridin-6-yl)-5-methyl-pyridine-3-carboxylate;
6-[3-(2-fluoro-5-methyl-4-pyridyl)-7,8-dihydro-5H-1,6-naphthyridin-6-yl]-4,5-dimethyl-pyridazine-3-carbonitrile;
6-[3-(2-chloro-3-fluoro-4-pyridyl)-7,8-dihydro-5H-1,6-naphthyridin-6-yl]-4,5-dimethyl-pyridazine-3-carbonitrile;
4,5-dimethyl-6-[3-(4-pyridyl)-7,8-dihydro-5H-1,6-naphthyridin-6-yl]pyridazine-3-carbonitrile;
6-[3-(2,6-difluoro-4-pyridyl)-7,8-dihydro-5H-1,6-naphthyridin-6-yl]-4,5-dimethyl-pyridazine-3-carbonitrile;
6-[3-(2-methoxy-4-pyridyl)-7,8-dihydro-5H-1,6-naphthyridin-6-yl]-4,5-dimethyl-pyridazine-3-carbonitrile;
4,5-dimethyl-6-[3-(3-methyl-4-pyridyl)-7,8-dihydro-5H-1,6-naphthyridin-6-yl]pyridazine-3-carbonitrile;
6-[3-[(3,5-difluoro-2-pyridyl)amino]-7,8-dihydro-5H-1,6-naphthyridin-6-yl]-4,5-dimethyl-pyridazine-3-carbonitrile;
6-[3-[(2-fluoro-6-methyl-3-pyridyl)amino]-7,8-dihydro-5H-1,6-naphthyridin-6-yl]-4,5-dimethyl-pyridazine-3-carbonitrile;
6-[3-[(3-fluoro-2-pyridyl)amino]-7,8-dihydro-5H-1,6-naphthyridin-6-yl]-4,5-dimethyl-pyridazine-3-carbonitrile;
6-[3-[(2-chloro-5-fluoro-pyrimidin-4-yl)amino]-7,8-dihydro-5H-1,6-naphthyridin-6-yl]-4,5-dimethyl-pyridazine-3-carbonitrile;
6-[3-[(5-chloro-2-fluoro-3-pyridyl)amino]-7,8-dihydro-5H-1,6-naphthyridin-6-yl]-4,5-dimethyl-pyridazine-3-carbonitrile;
6-(6-chloro-2,5-dimethyl-pyrimidin-4-yl)-7,8-dihydro-5H-1,6-naphthyridine;
6-(6-chloro-2-cyclopropyl-5-methyl-pyrimidin-4-yl)-7,8-dihydro-5H-1,6-naphthyridine;
6-(6-chloro-2,5-dimethyl-pyrimidin-4-yl)-N-(2-fluorophenyl)-7,8-dihydro-5H-1,6-naphthyridin-3-amine;
6-(6-chloro-2,5-dimethyl-pyrimidin-4-yl)-N-(2,3-difluorophenyl)-7,8-dihydro-5H-1,6-naphthyridin-3-amine;
6-(6-chloro-2,5-dimethyl-pyrimidin-4-yl)-N-(2,4-difluorophenyl)-7,8-dihydro-5H-1,6-naphthyridin-3-amine;
6-(6-chloro-2,5-dimethyl-pyrimidin-4-yl)-N-(2,5-difluorophenyl)-7,8-dihydro-5H-1,6-naphthyridin-3-amine;
6-(6-chloro-2,5-dimethyl-pyrimidin-4-yl)-N-(2,6-difluorophenyl)-7,8-dihydro-5H-1,6-naphthyridin-3-amine;
6-(6-chloro-2-cyclopropyl-5-methyl-pyrimidin-4-yl)-N-(2-fluorophenyl)-7,8-dihydro-5H-1,6-naphthyridin-3-amine;
6-(6-chloro-2-cyclopropyl-5-methyl-pyrimidin-4-yl)-N-(2,3-difluorophenyl)-7,8-dihydro-5H-1,6-naphthyridin-3-amine;
6-(6-chloro-2,5-dimethyl-pyrimidin-4-yl)-3-(1-methylpyrazol-4-yl)-7,8-dihydro-5H-1,6-naphthyridine;
6-(6-chloro-2,5-dimethyl-pyrimidin-4-yl)-3-(2-methyl-4-pyridyl)-7,8-dihydro-5H-1,6-naphthyridine;
5-[6-(6-chloro-2,5-dimethyl-pyrimidin-4-yl)-7,8-dihydro-5H-1,6-naphthyridin-3-yl]thiazole;
5-methyl-6-(3-thiazol-5-yl-7,8-dihydro-5H-1,6-naphthyridin-6-yl)pyridine-3-carbonitrile;
6-[3-(1-cyclopropylpyrazol-4-yl)-7,8-dihydro-5H-1,6-naphthyridin-6-yl]-5-methyl-pyridine-3-carbonitrile;
6-[3-(1-benzylpyrazol-4-yl)-7,8-dihydro-5H-1,6-naphthyridin-6-yl]-5-methyl-pyridine-3-carbonitrile;
6-[3-(1-cyclopentylpyrazol-4-yl)-7,8-dihydro-5H-1,6-naphthyridin-6-yl]-5-methyl-pyridine-3-carbonitrile;
6-[3-(1-cyclohexylpyrazol-4-yl)-7,8-dihydro-5H-1,6-naphthyridin-6-yl]-5-methyl-pyridine-3-carbonitrile;
5-methyl-6-[3-[1-(2,2,2-trifluoroethyl)pyrazol-4-yl]-7,8-dihydro-5H-1,6-naphthyridin-6-yl]pyridine-3-carbonitrile;
6-[3-(1-cyclobutylpyrazol-4-yl)-7,8-dihydro-5H-1,6-naphthyridin-6-yl]-5-methyl-pyridine-3-carbonitrile;
6-[3-[1-(cyclopropylmethyl)pyrazol-4-yl]-7,8-dihydro-5H-1,6-naphthyridin-6-yl]-5-methyl-pyridine-3-carbonitrile;
6-[3-(1,3-dimethylpyrazol-4-yl)-7,8-dihydro-5H-1,6-naphthyridin-6-yl]-5-methyl-pyridine-3-carbonitrile;
5-methyl-6-[3-(1-methylpyrazol-4-yl)-7,8-dihydro-5H-1,6-naphthyridin-6-yl]pyridine-3-carbonitrile;
6-[3-(1-ethylpyrazol-4-yl)-7,8-dihydro-5H-1,6-naphthyridin-6-yl]-5-methyl-pyridine-3-carbonitrile;
6-[3-[1-(4-fluorophenyl)pyrazol-4-yl]-7,8-dihydro-5H-1,6-naphthyridin-6-yl]-5-methyl-pyridine-3-carbonitrile;
5-methyl-6-[3-(1,3,5-trimethylpyrazol-4-yl)-7,8-dihydro-5H-1,6-naphthyridin-6-yl]pyridine-3-carbonitrile;
5-methyl-6-[3-(1-propylpyrazol-4-yl)-7,8-dihydro-5H-1,6-naphthyridin-6-yl]pyridine-3-carbonitrile;
6-[3-(3,5-dimethylisoxazol-4-yl)-7,8-dihydro-5H-1,6-naphthyridin-6-yl]-5-methyl-pyridine-3-carbonitrile;
6-[3-(2,5-difluoroanilino)-7,8-dihydro-5H-1,6-naphthyridin-6-yl]-5-methyl-pyridine-3-carbonitrile;
6-[3-(2-chloro-6-fluoro-anilino)-7,8-dihydro-5H-1,6-naphthyridin-6-yl]-5-methyl-pyridine-3-carbonitrile;
6-[3-(1-isobutylpyrazol-4-yl)-7,8-dihydro-5H-1,6-naphthyridin-6-yl]-5-methyl-pyridine-3-carbonitrile;
6-[3-[2-fluoro-3-(trifluoromethyl)anilino]-7,8-dihydro-5H-1,6-naphthyridin-6-yl]-5-methyl-pyridine-3-carbonitrile;
6-[3-(3-chloro-2-fluoro-anilino)-7,8-dihydro-5H-1,6-naphthyridin-6-yl]-5-methyl-pyridine-3-carbonitrile;
6-[3-(2-fluoro-5-methyl-anilino)-7,8-dihydro-5H-1,6-naphthyridin-6-yl]-5-methyl-pyridine-3-carbonitrile;
5-methyl-6-[3-(2,3,5-trifluoroanilino)-7,8-dihydro-5H-1,6-naphthyridin-6-yl]pyridine-3-carbonitrile;
4,5-dimethyl-6-(3-methyl-7,8-dihydro-5H-1,6-naphthyridin-6-yl)pyridazine-3-carbonitrile;
6-(7,8-dihydro-5H-1,6-naphthyridin-6-yl)-4,5-dimethyl-pyridazine-3-carbonitrile;
6-[3-[(2-fluoro-3-pyridyl)amino]-7,8-dihydro-5H-1,6-naphthyridin-6-yl]-4,5-dimethyl-pyridazine-3-carbonitrile;
6-(7,8-dihydro-5H-1,6-naphthyridin-6-yl)-N-(imidazo[1,2-a]pyridin-2-ylmethyl)-5-methyl-pyridine-3-carboxamide;
6-(7,8-dihydro-5H-1,6-naphthyridin-6-yl)-5-methyl-N-(4-pyridyl)pyridine-3-carboxamide;
6-(7,8-dihydro-5H-1,6-naphthyridin-6-yl)-5-methyl-N-tetrahydropyran-4-yl-pyridine-3-carboxamide;
6-(7,8-dihydro-5H-1,6-naphthyridin-6-yl)-N-(isoxazol-5-ylmethyl)-5-methyl-pyridine-3-carboxamide;
6-(7,8-dihydro-5H-1,6-naphthyridin-6-yl)-N-(3-fluoro-1-bicyclo[1.1.1]pentanyl)-5-methyl-pyridine-3-carboxamide;
N-(1,3-benzothiazol-2-ylmethyl)-6-(7,8-dihydro-5H-1,6-naphthyridin-6-yl)-5-methyl-pyridine-3-carboxamide;
6-(7,8-dihydro-5H-1,6-naphthyridin-6-yl)-5-methyl-N-[2-(4-pyridyl)ethyl]pyridine-3-carboxamide;
6-(7,8-dihydro-5H-1,6-naphthyridin-6-yl)-5-methyl-N-[(2-methylthiazol-5-yl)methyl]pyridine-3-carboxamide;
N-(benzothiophen-3-ylmethyl)-6-(7,8-dihydro-5H-1,6-naphthyridin-6-yl)-5-methyl-pyridine-3-carboxamide;
6-(7,8-dihydro-5H-1,6-naphthyridin-6-yl)-5-methyl-N-[(2-methyl-4-pyridyl)methyl]pyridine-3-carboxamide;
6-(7,8-dihydro-5H-1,6-naphthyridin-6-yl)-5-methyl-N-[(3-methyl-4-pyridyl)methyl]pyridine-3-carboxamide;
6-(7,8-dihydro-5H-1,6-naphthyridin-6-yl)-5-methyl-N-(4-quinolylmethyl)pyridine-3-carboxamide;
6-[3-[(2-fluoro-6-methyl-3-pyridyl)amino]-7,8-dihydro-5H-1,6-naphthyridin-6-yl]-5-methyl-pyridine-3-carbonitrile;
methyl 6-(3-bromo-7,8-dihydro-5H-1,6-naphthyridin-6-yl)-5-methyl-pyridine-3-carboxylate;
N-[(2-chlorothiazol-5-yl)methyl]-6-(7,8-dihydro-5H-1,6-naphthyridin-6-yl)-5-methyl-pyridine-3-carboxamide;
6-(7,8-dihydro-5H-1,6-naphthyridin-6-yl)-4,5-dimethyl-N-(4-pyridylmethyl)pyridazine-3-carboxamide;
6-(7,8-dihydro-5H-1,6-naphthyridin-6-yl)-4,5-dimethyl-N-(3-pyridylmethyl)pyridazine-3-carboxamide;
6-(7,8-dihydro-5H-1,6-naphthyridin-6-yl)-4,5-dimethyl-N-(2-pyridylmethyl)pyridazine-3-carboxamide;
6-(7,8-dihydro-5H-1,6-naphthyridin-6-yl)-4,5-dimethyl-N-(thiazol-5-ylmethyl)pyridazine-3-carboxamide;
N-[(5-bromo-3-pyridyl)methyl]-6-(7,8-dihydro-5H-1,6-naphthyridin-6-yl)-5-methyl-pyridine-3-carboxamide;
6-(7,8-dihydro-5H-1,6-naphthyridin-6-yl)-5-methyl-N-(3-quinolylmethyl)pyridine-3-carboxamide;
N-[(5-bromo-2-methoxy-phenyl)methyl]-6-(7,8-dihydro-5H-1,6-naphthyridin-6-yl)-5-methyl-pyridine-3-carboxamide;
methyl 5-[[[6-(7,8-dihydro-5H-1,6-naphthyridin-6-yl)-5-methyl-pyridine-3-carbonyl]amino]methyl]pyridine-2-carboxylate;
6-(7,8-dihydro-5H-1,6-naphthyridin-6-yl)-5-methyl-N-[(4-methylsulfonylphenyl)methyl]pyridine-3-carboxamide;
6-(7,8-dihydro-5H-1,6-naphthyridin-6-yl)-5-methyl-N-[[5-(trifluoromethyl)-2-pyridyl]methyl]pyridine-3-carboxamide;
6-(7,8-dihydro-5H-1,6-naphthyridin-6-yl)-5-methyl-N-[[5-(trifluoromethyl)pyrimidin-2-yl]methyl]pyridine-3-carboxamide;
N-[(5-chloropyrimidin-2-yl)methyl]-6-(7,8-dihydro-5H-1,6-naphthyridin-6-yl)-5-methyl-pyridine-3-carboxamide;
6-(7,8-dihydro-5H-1,6-naphthyridin-6-yl)-N-[(5-fluoropyrimidin-2-yl)methyl]-5-methyl-pyridine-3-carboxamide;
6-(7,8-dihydro-5H-1,6-naphthyridin-6-yl)-N-[(6-imidazol-1-yl-3-pyridyl)methyl]-5-methyl-pyridine-3-carboxamide;
6-(7,8-dihydro-5H-1,6-naphthyridin-6-yl)-5-methyl-N-[[4-(methylcarbamoyl)phenyl]methyl]pyridine-3-carboxamide;
N-(3H-benzotriazol-5-ylmethyl)-6-(7,8-dihydro-5H-1,6-naphthyridin-6-yl)-5-methyl-pyridine-3-carboxamide;
6-(7,8-dihydro-5H-1,6-naphthyridin-6-yl)-5-methyl-N-([1,2,4]triazolo[1,5-a]pyridin-6-ylmethyl)pyridine-3-carboxamide;
6-(7,8-dihydro-5H-1,6-naphthyridin-6-yl)-N-[(4-fluoro-3-methoxy-phenyl)methyl]-5-methyl-pyridine-3-carboxamide;
N-(1,3-dihydroisobenzofuran-5-ylmethyl)-6-(7,8-dihydro-5H-1,6-naphthyridin-6-yl)-5-methyl-pyridine-3-carboxamide;
6-(7,8-dihydro-5H-1,6-naphthyridin-6-yl)-5-methyl-N-[(2-oxoindolin-5-yl)methyl]pyridine-3-carboxamide;
N-(6,7-dihydro-5H-cyclopenta[b]pyridin-3-ylmethyl)-6-(7,8-dihydro-5H-1,6-naphthyridin-6-yl)-5-methyl-pyridine-3-carboxamide;
6-(7,8-dihydro-5H-1,6-naphthyridin-6-yl)-5-methyl-N-(8-quinolylmethyl)pyridine-3-carboxamide;
6-(7,8-dihydro-5H-1,6-naphthyridin-6-yl)-5-methyl-N-(quinoxalin-6-ylmethyl)pyridine-3-carboxamide;
6-(7,8-dihydro-5H-1,6-naphthyridin-6-yl)-N-[(1,3-dimethyl-2-oxo-benzimidazol-5-yl)methyl]-5-methyl-pyridine-3-carboxamide;
6-(7,8-dihydro-5H-1,6-naphthyridin-6-yl)-N-[(3-fluorophenyl)methyl]-5-methyl-pyridine-3-carboxamide;
6-(7,8-dihydro-5H-1,6-naphthyridin-6-yl)-5-methyl-N-(tetrahydropyran-4-ylmethyl)pyridine-3-carboxamide;
6-(7,8-dihydro-5H-1,6-naphthyridin-6-yl)-5-methyl-N-(tetrahydropyran-2-ylmethyl)pyridine-3-carboxamide;
6-(7,8-dihydro-5H-1,6-naphthyridin-6-yl)-N-[(7-methoxybenzofuran-2-yl)methyl]-5-methyl-pyridine-3-carboxamide;
6-(7,8-dihydro-5H-1,6-naphthyridin-6-yl)-5-methyl-N-(1H-pyrrol-2-ylmethyl)pyridine-3-carboxamide;
6-(7,8-dihydro-5H-1,6-naphthyridin-6-yl)-N-[(1,1-dioxo-2,3-dihydrobenzothiophen-5-yl)methyl]-5-methyl-pyridine-3-carboxamide;
5-methyl-6-[3-(trifluoromethyl)-7,8-dihydro-5H-1,6-naphthyridin-6-yl]pyridine-3-carbonitrile;
6-[3-[(2-fluoro-3-pyridyl)amino]-7,8-dihydro-5H-1,6-naphthyridin-6-yl]-5-methyl-pyridine-3-carbonitrile;
6-[3-[(3-fluoro-2-pyridyl)amino]-7,8-dihydro-5H-1,6-naphthyridin-6-yl]-5-methyl-pyridine-3-carbonitrile;
6-[3-(6-fluoro-2-pyridyl)-7,8-dihydro-5H-1,6-naphthyridin-6-yl]-5-methyl-pyridine-3-carbonitrile;
4,5-dimethyl-6-[3-[(2-methylpyrazol-3-yl)amino]-7,8-dihydro-5H-1,6-naphthyridin-6-yl]pyridazine-3-carbonitrile;
6-[3-(1,3-dimethylpyrazol-4-yl)-7,8-dihydro-5H-1,6-naphthyridin-6-yl]-5-methyl-N-(thiazol-2-ylmethyl)pyridine-3-carboxamide;
5-methyl-6-[3-(1-methylpyrazol-3-yl)-7,8-dihydro-5H-1,6-naphthyridin-6-yl]-N-(thiazol-2-ylmethyl)pyridine-3-carboxamide;
6-[3-(3,5-dimethylisoxazol-4-yl)-7,8-dihydro-5H-1,6-naphthyridin-6-yl]-5-methyl-N-(thiazol-2-ylmethyl)pyridine-3-carboxamide;
5-methyl-6-[3-(1-methylpyrazol-4-yl)-7,8-dihydro-5H-1,6-naphthyridin-6-yl]-N-(thiazol-2-ylmethyl)pyridine-3-carboxamide;
6-[3-(6-fluoro-4-methyl-3-pyridyl)-7,8-dihydro-5H-1,6-naphthyridin-6-yl]-5-methyl-N-(thiazol-2-ylmethyl)pyridine-3-carboxamide;
6-[3-(2-fluoro-3-pyridyl)-7,8-dihydro-5H-1,6-naphthyridin-6-yl]-5-methyl-N-(thiazol-2-ylmethyl)pyridine-3-carboxamide;
5-methyl-6-[3-(1-methylpyrazol-4-yl)-7,8-dihydro-5H-1,6-naphthyridin-6-yl]-N-(4-pyridylmethyl)pyridine-3-carboxamide;
6-[3-(6-fluoro-4-methyl-3-pyridyl)-7,8-dihydro-5H-1,6-naphthyridin-6-yl]-5-methyl-N-(4-pyridylmethyl)pyridine-3-carboxamide;
6-[3-(2-fluoro-3-pyridyl)-7,8-dihydro-5H-1,6-naphthyridin-6-yl]-5-methyl-N-(4-pyridylmethyl)pyridine-3-carboxamide;
5-methyl-6-(3-methyl-7,8-dihydro-5H-1,6-naphthyridin-6-yl)pyridine-3-carbonitrile;
6-[3-(2-fluorophenoxy)-7,8-dihydro-5H-1,6-naphthyridin-6-yl]-5-methyl-pyridine-3-carbonitrile;
6-[3-(3-fluorophenoxy)-7,8-dihydro-5H-1,6-naphthyridin-6-yl]-5-methyl-pyridine-3-carbonitrile;
6-[3-[(6-methoxy-3-pyridyl)oxy]-7,8-dihydro-5H-1,6-naphthyridin-6-yl]-5-methyl-pyridine-3-carbonitrile;
5-methyl-6-[3-(1-methylindazol-5-yl)oxy-7,8-dihydro-5H-1,6-naphthyridin-6-yl]pyridine-3-carbonitrile;
6-[3-(2,4-dimethylthiazol-5-yl)-7,8-dihydro-5H-1,6-naphthyridin-6-yl]-5-methyl-pyridine-3-carbonitrile;
5-methyl-6-[3-(2-methylthiazol-5-yl)-7,8-dihydro-5H-1,6-naphthyridin-6-yl]pyridine-3-carbonitrile;
6-[3-(6-cyclopropyl-3-pyridyl)-7,8-dihydro-5H-1,6-naphthyridin-6-yl]-5-methyl-pyridine-3-carbonitrile;
5-methyl-6-[3-(6-methyl-3-pyridyl)-7,8-dihydro-5H-1,6-naphthyridin-6-yl]pyridine-3-carbonitrile;
6-[3-(6-methoxy-3-pyridyl)-7,8-dihydro-5H-1,6-naphthyridin-6-yl]-5-methyl-pyridine-3-carbonitrile;
4,5-dimethyl-6-[3-(2-methyl-4-pyridyl)-7,8-dihydro-5H-1,6-naphthyridin-6-yl]-N-(4-pyridylmethyl)pyridazine-3-carboxamide;
6-[3-(2-fluoroanilino)-7,8-dihydro-5H-1,6-naphthyridin-6-yl]-4,5-dimethyl-N-(4-pyridylmethyl)pyridazine-3-carboxamide;
5-methyl-6-[3-(3-pyridyl)-7,8-dihydro-5H-1,6-naphthyridin-6-yl]pyridine-3-carbonitrile;
6-(6-chloro-2,5-dimethyl-pyrimidin-4-yl)-3-(1,3-dimethylpyrazol-4-yl)-7,8-dihydro-5H-1,6-naphthyridine;
6-(6-chloro-2,5-dimethyl-pyrimidin-4-yl)-3-(1,3,5-trimethylpyrazol-4-yl)-7,8-dihydro-5H-1,6-naphthyridine;
6-(6-chloro-2,5-dimethyl-pyrimidin-4-yl)-3-[1-(cyclopropylmethyl)pyrazol-4-yl]-7,8-dihydro-5H-1,6-naphthyridine;
6-(6-chloro-2,5-dimethyl-pyrimidin-4-yl)-3-(1-cyclobutylpyrazol-4-yl)-7,8-dihydro-5H-1,6-naphthyridine;
3-(1,3-dimethylpyrazol-4-yl)-6-(6-methoxy-2,5-dimethyl-pyrimidin-4-yl)-7,8-dihydro-5H-1,6-naphthyridine;
4-[6-(6-chloro-2,5-dimethyl-pyrimidin-4-yl)-7,8-dihydro-5H-1,6-naphthyridin-3-yl]-3,5-dimethyl-isoxazole;
5-[6-(6-chloro-2,5-dimethyl-pyrimidin-4-yl)-7,8-dihydro-5H-1,6-naphthyridin-3-yl]-2,4-dimethyl-thiazole;
5-[6-(6-chloro-2,5-dimethyl-pyrimidin-4-yl)-7,8-dihydro-5H-1,6-naphthyridin-3-yl]-2-methyl-thiazole;
6-(6-chloro-2,5-dimethyl-pyrimidin-4-yl)-3-(trifluoromethyl)-7,8-dihydro-5H-1,6-naphthyridine;
3-(1,3-dimethylpyrazol-4-yl)-6-(2,5,6-trimethylpyrimidin-4-yl)-7,8-dihydro-5H-1,6-naphthyridine;
6-(6-chloro-2,5-dimethyl-pyrimidin-4-yl)-3-(4-fluorophenoxy)-7,8-dihydro-5H-1,6-naphthyridine;
6-(6-chloro-2,5-dimethyl-pyrimidin-4-yl)-3-(3-fluorophenoxy)-7,8-dihydro-5H-1,6-naphthyridine;
6-(6-chloro-2,5-dimethyl-pyrimidin-4-yl)-3-(2-fluorophenoxy)-7,8-dihydro-5H-1,6-naphthyridine;
6-(6-chloro-2,5-dimethyl-pyrimidin-4-yl)-3-[(6-methoxy-3-pyridyl)oxy]-7,8-dihydro-5H-1,6-naphthyridine;
6-(6-chloro-2,5-dimethyl-pyrimidin-4-yl)-3-(2,3-dihydro-1,4-benzodioxin-6-yloxy)-7,8-dihydro-5H-1,6-naphthyridine;
6-(6-chloro-2,5-dimethyl-pyrimidin-4-yl)-3-(3-pyridyloxy)-7,8-dihydro-5H-1,6-naphthyridine;
6-(6-chloro-2,5-dimethyl-pyrimidin-4-yl)-3-(1-methylindazol-5-yl)oxy-7,8-dihydro-5H-1,6-naphthyridine; and
6-(6-chloro-2,5-dimethyl-pyrimidin-4-yl)-3-methyl-7,8-dihydro-5H-1,6-naphthyridine;

or a pharmaceutically acceptable salt thereof.

In the following, numbered embodiments of the invention are disclosed. The first embodiment is denoted E1, subsequent embodiments are denoted E1.1, E2, and so forth.
E1. A compound of formula (I), or a pharmaceutically acceptable salt thereof,

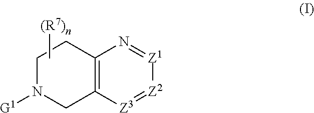

wherein:

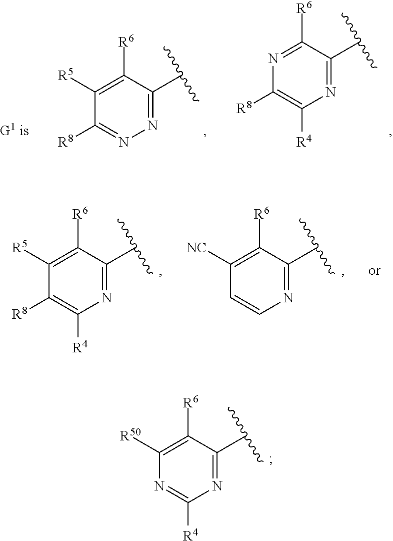

Z¹ is N or CR¹;
Z² is CR²;
Z³ is N or CR³;
R¹ is hydrogen, halogen, cyano, C₁₋₆alkyl, C₁₋₆haloalkyl, —OR^{H}, —R^{c}, —SR^{b}, —OC(O)R^{b}, —NR^{b}C(O)R^{c}, —NR^{b}SO₂R^{a}, —C(O)OR^{b}, —C(O)NR^{b}R^{c}, —SO₂NR^{b}R^{c}, —C(O)R^{b}, —S(O)R^{a}, —SO₂R^{a}, G², or —C₁₋₃alkylene-G²;
R² and R³ are each independently hydrogen, halogen, cyano, NO₂, C₁₋₆alkyl, C₁₋₆haloalkyl, —OR^{b}, —R^{c}, —SR^{b}, —OC(O)R^{b}, —NR^{b}C(O)R^{c}, —NR^{b}SO₂R^{a}, —C(O)OR^{b}, —C(O)NR^{b}R^{c}, —SO₂NR^{b}R^{c}, —C(O)R^{b}, —S(O)R^{a}, —SO₂R^{a}, —C₁₋₆alkylene-OH, —C₁₋₆fluoroalkylene-OH, G², or —C₁₋₃alkylene-G²;
R^{a}, at each occurrence, is independently C₁₋₆alkyl, C₁₋₆haloalkyl, G², or —C₁₋₃alkylene-G²;
R^{b} and R^{c}, at each occurrence, are independently hydrogen, C₁₋₆alkyl, C₁₋₆haloalkyl, G², —C₁₋₃alkylene-G², —C₂₋₄alkylene-O—C₁₋₄alkyl, or —C₂₋₄alkylene-O—G²;
R^{H} is hydrogen, C₁₋₆alkyl, C₁₋₆haloalkyl, G^{2H}, or —C₁₋₃alkylene-G^{2H};
G², at each occurrence, is independently a 6- to 12-membered aryl, a 5- to 12-membered heteroaryl, a 4- to 12-membered heterocyclyl, or a 3- to 12-membered carbocyclyl, wherein G² is optionally substituted with 1-5 substituents independently selected from the group consisting of halogen, cyano, C₁₋₆alkyl, C₁₋₆haloalkyl, oxo, —OR^{x}, —N(R^{x})₂, —C₁₋₆alkylene-OR^{x}, —C₁₋₆alkylene-N(R^{x})₂, G^{2a}, and —C₁₋₃alkylene-G^{2a};
G^{2H} is a 6- to 12-membered aryl, a 5- to 12-membered heteroaryl, a 4- to 8-membered heterocyclyl containing 1-2 oxygen atoms, or a 3- to 12-membered carbocyclyl, wherein G^{2H} is optionally substituted with 1-5 substituents independently selected from the group consisting of halogen, cyano, C₁₋₄alkyl, C₁₋₄haloalkyl, oxo, —OR^{x}, —N(R^{x})₂, G^{2a}, and —C₁₋₃alkylene-G^{2a};
R^{x}, at each occurrence, is independently hydrogen, C₁₋₄alkyl, C₁₋₄haloalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄haloalkyl;
R⁴ is hydrogen, halogen, cyano, C₁₋₆alkyl, C₁₋₆haloalkyl, —C₁₋₆alkylene-OR^{4a}, —OR^{4a}, —O—C₂₋₆alkylene-OR^{4a}, —N(R^{4a})₂, —N(R^{4a})—C₂₋₆alkylene-OR^{4a}, G³, —O—G³, —N(R^{4a})—G³, —C₁₋₃alkylene-G³, —O—C₁₋₃alkylene-G³, or —N(R^{4a})—C₁₋₃alkylene-G³;
R^{4a}, at each occurrence, is independently hydrogen, C₁₋₆alkyl, or C₁₋₆haloalkyl;
R⁵ and R⁶ are each independently hydrogen, halogen, cyano, C₁₋₆alkyl, C₁₋₆haloalkyl, OH, —OC₁₋₆alkyl, —OC₁₋₆haloalkyl, G³, —O—G³, —C₁₋₃alkylene-G³, or —O—C₁₋₃alkylene-G³;
R⁵⁰ is hydrogen, halogen, cyano, C₁₋₆alkyl, C₁₋₆haloalkyl, OH, —OC₁₋₆alkyl, —OC₁₋₆haloalkyl, G³⁰, —O—G³, —C(O)—N(R^{50a}, —C₁₋₃alkylene-G³, or —O—C₁₋₃alkylene-G³;
R^{50a}, at each occurrence, is independently hydrogen, C₁₋₄alkyl, G³, or —C₁₋₃alkylene-G³, or two R^{50a}, together with the nitrogen to which they attach, form a 4- to 8-membered heterocyclyl, the heterocyclyl optionally containing a second heteroatom that is O, N, or S and being optionally substituted with 1-4 C₁₋₄alkyl;
wherein, alternatively, R⁵⁰ and R⁶, together with the atom to which each attaches, form a 5- to 7-membered non-aromatic carbocyclic or heterocyclic ring, the heterocyclic ring containing one heteroatom that is O, N, or S and the carbocyclic or heterocyclic ring being optionally substituted with 1-4 C₁₋₄alkyl;
G³, at each occurrence, is independently a 6- to 12-membered aryl, a 5- to 12-membered heteroaryl, a 4- to 12-membered heterocyclyl, or a 3- to 12-membered carbocyclyl, wherein G³ is optionally substituted with 1-5 substituents independently selected from the group consisting of halogen, cyano, C₁₋₄alkyl, C₁₋₄haloalkyl, oxo, OH, —OC₁₋₄alkyl, —OC₁₋₄haloalkyl, G^{3a}, and —C₁₋₃alkylene-G^{3a};
G³⁰ is a 6- to 12-membered aryl, a 5- to 12-membered heteroaryl other than indazol-3-yl, a 4- to 12-membered heterocyclyl, or a 3- to 12-membered carbocyclyl, wherein G³⁰ is optionally substituted with 1-5 substituents independently selected from the group consisting of halogen, cyano, C₁₋₄alkyl, C₁₋₄haloalkyl, oxo, OH, —OC₁₋₄alkyl, —OC₁₋₄haloalkyl, G^{3a}, and —C₁₋₃alkylene-G^{3a};
R⁷, at each occurrence, is independently halogen, C₁₋₄alkyl, C₁₋₄haloalkyl, C₃₋₇cycloalkyl, OH, —OC₁₋₄alkyl, or —OC₁₋₄haloalkyl, wherein the cycloalkyl is optionally substituted with 1-4 substituents independently selected from the group consisting of halogen, C₁₋₄alkyl, and C₁₋₄haloalkyl;
R⁸ is halogen, cyano, C₁₋₆alkyl, C₁₋₆haloalkyl, —OR^{8b}, —NR^{8b}R^{8c}, —SR^{8b}, —OC(O)R^{8b}, —NR^{8b}C(O)R^{8c}—NR^{8b}SO₂R^{8a}, —C(O)OR^{8b}, —C(O)NR^{8b}R^{8c}, —SO₂NR^{8b}R^{8c}, —C(O)R^{8b}—S(O)R^{8a}, —SO₂R^{8a}, G⁴, or —C₁₋₃alkylene-G⁴;
R^{8a}, at each occurrence, is independently C₁₋₆alkyl, C₁₋₆haloalkyl, G⁴, or —C₁₋₃alkylene-G⁴;
R^{8b} and R^{8c}, at each occurrence, are independently hydrogen, C₁₋₆alkyl, C₁₋₆haloalkyl, —C₁₋₆alkylene-OH, G⁴, or —C₁₋₃alkylene-G⁴;
wherein, alternatively, when R⁸ is —C(O)NR^{8b}R^{8c} and R⁴ is —OR^{4a}, R^{8c} and R^{4a}, together with the atom to which each attaches, form an oxazepin-5-one ring;
G⁴, at each occurrence, is independently a 6- to 12-membered aryl, a 5- to 12-membered heteroaryl, a 4- to 12-membered heterocyclyl, or a 3- to 12-membered carbocyclyl optionally fused to a 6-membered arene or heteroarene that contains 1-2 nitrogen atoms, wherein G⁴ is optionally substituted with 1-5 substituents independently selected from the group consisting of halogen, cyano, C₁₋₄alkyl, C₁₋₄haloalkyl, —C₁₋₆alkylene-OH, oxo, —OR⁸⁰, —N(R⁸⁰)₂, —NR⁸⁰C(O)R⁸⁰, —NR⁸⁰S02R⁸⁰, —C(O)OR⁸⁰, —C(O)N(R⁸⁰)₂, —SO₂R⁸⁰, G^{4a}, and —C₁₋₃alkylene-G^{4a}.
R⁸⁰, at each occurrence, is independently hydrogen, C₁₋₄alkyl, C₁₋₄haloalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄haloalkyl;
G^{2a}, G^{3a}, and G^{4a}, at each occurrence, are independently a phenyl, a 5- to 6-membered heteroaryl, a 4- to 8-membered heterocyclyl, or a 3- to 8-membered carbocyclyl, wherein G^{2a}, G^{3a}, and G^{4a}, at each occurrence, are independently optionally substituted with 1-5 substituents independently selected from the group consisting of halogen, cyano, C₁₋₄alkyl, C₁₋₄haloalkyl, —C₁₋₆alkylene-OH, oxo, OH, —OC₁₋₄alkyl, —OC₁₋₄haloalkyl, C₃₋₄cycloalkyl, and —C₁₋₃alkylene-C₃₋₄cycloalkyl;
and
n is 0, 1, 2, 3, or 4;
provided that the compound is not
6-[3-chloro-5-(trifluoromethyl)-2-pyridinyl]-5,6,7,8-tetrahydro-3-methoxy-pyrido[4,3-c]pyridazine;
6-(7,8-dihydro-1,6-naphthyridin-6(5H)-yl)-N-[2-(1-methyl-2-piperidinyl)ethyl]-3-pyridinecarboxamide;
6-(7,8-dihydro-1,6-naphthyridin-6(5H)-yl)-N-[(3-propyl-5-isoxazolyl)methyl]-3-pyridinecarboxamide;
6-(6-methyl-2-(pyridin-3-yl)pyrimidin-4-yl)-5, 6,7,8-tetrahydro-1,6-naphthyridine;
4-[7,8-dihydro-3-(1-pyrrolidinyl)pyrido[4,3-c]pyridazin-6(5H)-yl]-2-pyrimidinecarbonitrile;
4-(7,8-dihydro-3-methoxypyrido[4,3-c]pyridazin-6(5H)-yl)-2-pyrimidinecarbonitrile;
6-(2,6-dimethyl-4-pyrimidinyl)-5,6,7,8-tetrahydro-1,6-naphthyridin-4-amine;
5,6,7,8-tetrahydro-6-[2-(1-piperidinyl)-4-pyrimidinyl]-pyrido[4,3-c]pyridazin-3(2H)-one; or
5,6,7,8-tetrahydro-6-[6-(1-piperidinyl)-4-pyrimidinyl]-pyrido[4,3-c]pyridazin-3(2H)-one, or a salt thereof.
   E1.1. The compound of E1, or a pharmaceutically acceptable salt thereof, wherein
   G¹ is

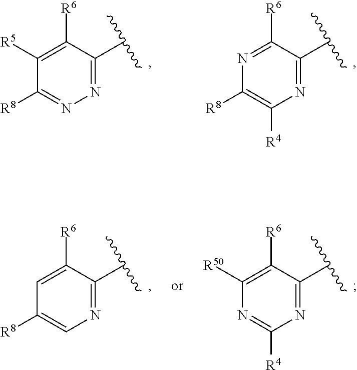

R² and R³ are each independently hydrogen, halogen, cyano, C₁₋₆alkyl, C₁₋₆haloalkyl, —OR^{b}, —R^{b}R^{c}, —SR^{b}, —OC(O)R^{b}, —NR^{b}C(O)R^{c}, —NR^{b}SO₂R^{a}, —C(O)OR^{b}, —C(O)NR^{b}R^{c}, —SO₂NR^{b}R^{c}, —C(O)R^{b}, —S(O)R^{a}, —SO₂R^{a}, G², or —C₁₋₃alkylene-G²;
R^{b} and R^{c}, at each occurrence, are independently hydrogen, C₁₋₆alkyl, C₁₋₆haloalkyl, G², or —C₁₋₃alkylene-G²;
G², at each occurrence, is independently a 6- to 12-membered aryl, a 5- to 12-membered heteroaryl, a 4- to 12-membered heterocyclyl, or a 3- to 12-membered carbocyclyl, wherein G² is optionally substituted with 1-5 substituents independently selected from the group consisting of halogen, cyano, C₁₋₄alkyl, C₁₋₄haloalkyl, oxo, —OR^{x}, —N(R^{x})₂, G^{2a}, and —C₁₋₃alkylene-G^{2a};
R⁴, R⁵ and R⁶ are each independently hydrogen, halogen, cyano, C₁₋₆alkyl, C₁₋₆haloalkyl, OH, —OC₁₋₆alkyl, —OC₁₋₆haloalkyl, G³, —O—G³, —C₁₋₃alkylene-G³, or —O—C₁₋₃alkylene-G³;
R⁵⁰ is hydrogen, halogen, cyano, C₁₋₆alkyl, C₁₋₆haloalkyl, OH, —OC₁₋₆alkyl, —OC₁₋₆haloalkyl, G³⁰, —O—G³, —C₁₋₃alkylene-G³, or —O—C₁₋₃alkylene-G³;
R^{8b} and R^{8c}, at each occurrence, are independently hydrogen, C₁₋₆alkyl, C₁₋₆haloalkyl, G⁴, or —C₁₋₃alkylene-G⁴;
G⁴, at each occurrence, is independently a 6- to 12-membered aryl, a 5- to 12-membered heteroaryl, a 4- to 12-membered heterocyclyl, or a 3- to 12-membered carbocyclyl, wherein G⁴ is optionally substituted with 1-5 substituents independently selected from the group consisting of halogen, cyano, C₁₋₄alkyl, C₁₋₄haloalkyl, oxo, —OR⁸⁰, —N(R⁸⁰)₂, —NR⁸⁰C(O)R⁸⁰, —NR⁸⁰ SO₂R⁸⁰, —C(O)OR⁸⁰, —C(O)N(R⁸⁰)₂, —SO₂R⁸⁰, G^{4a}, and —C₁₋₃alkylene-G⁴a; and
G^{2a}, G^{3a}, and G^{4a}, at each occurrence, are independently a phenyl, a 5- to 6-membered heteroaryl, a 4- to 8-membered heterocyclyl, or a 3- to 8-membered carbocyclyl, wherein G^{2a}, G^{3a}, and G^{4a}, at each occurrence, are independently optionally substituted with 1-5 substituents independently selected from the group consisting of halogen, cyano, C₁₋₄alkyl, C₁₋₄haloalkyl, oxo, OH, —OC₁₋₄alkyl, and —OC₁₋₄haloalkyl.
E2. The compound of E1 or E1.1, or a pharmaceutically acceptable salt thereof, wherein:
G¹ is

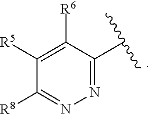

E3. The compound of any of E1-E2, or a pharmaceutically acceptable salt thereof, wherein R⁵ is hydrogen or C₁₋₄alkyl.
E3.1. The compound of E3, or a pharmaceutically acceptable salt thereof, wherein R⁵ is hydrogen.
E3.2. The compound of E3, or a pharmaceutically acceptable salt thereof, wherein R⁵ is C₁₋₄alkyl.
E3.3. The compound of E3.2, or a pharmaceutically acceptable salt thereof, wherein R⁵ is methyl.
E4. The compound of any of E1-E3.3, or a pharmaceutically acceptable salt thereof, wherein R⁶ is C₁₋₄alkyl.
E4.1. The compound of E4, or a pharmaceutically acceptable salt thereof, wherein R⁶ is methyl.
E4.2. The compound of E4, or a pharmaceutically acceptable salt thereof, wherein R⁵ and R⁶ are independently C₁₋₄alkyl.
E4.3. The compound of E4.1 or E4.2, or a pharmaceutically acceptable salt thereof, wherein R⁵ and R⁶ are CH₃.
E5. The compound of E1 or E1.1, or a pharmaceutically acceptable salt thereof, wherein
G¹ is

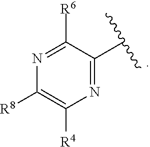

E6. The compound of any of E1, E1.1, or E5, or a pharmaceutically acceptable salt thereof, wherein R⁴ is hydrogen.
E7. The compound of E1 or E1.1, or a pharmaceutically acceptable salt thereof, wherein G¹ is

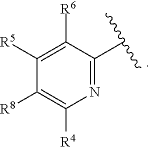

E7.1. The compound of E7, or a pharmaceutically acceptable salt thereof, wherein R⁴ is hydrogen, —OC₁₋₄alkyl (e.g., —OCH₃, —OCH₂CH₃), or a 5- to 6-membered heteroaryl containing 1-3 heteroatoms that are independently O, N, or S (e.g., pyrrol-2-yl), the heteroaryl being optionally substituted with 1-3 C₁₋₄alkyl (e.g., methyl).
E7.2. The compound of E7.1, or a pharmaceutically acceptable salt thereof, wherein R⁴ is hydrogen.
E7.3. The compound of any of E7-E7.2, or a pharmaceutically acceptable salt thereof, wherein R⁵ is hydrogen.
E7.4. The compound of any of E7-E7.3, or a pharmaceutically acceptable salt thereof, wherein:
G¹ is

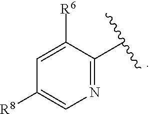

E8. The compound of any of E1, E1.1, or E7-E7.4, or a pharmaceutically acceptable salt thereof, wherein R⁶ is halogen, C₁₋₄alkyl, or C₁₋₄fluoroalkyl.
E8.1. The compound of E8, or a pharmaceutically acceptable salt thereof, wherein R⁶ is C₁₋₄alkyl.
E8.2. The compound of E8, or a pharmaceutically acceptable salt thereof, wherein R⁶ is bromo, CH₃, or CF³.
E8.3. The compound of any of E8-E8.2, or a pharmaceutically acceptable salt thereof, wherein R⁶ is CH₃.
E9. The compound of any of E1, E1.1, or E8-E8.3, or a pharmaceutically acceptable salt thereof, wherein G¹ is

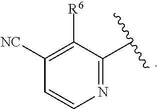

E10. The compound of E1 or E1.1, or a pharmaceutically acceptable salt thereof, wherein:
G¹ is

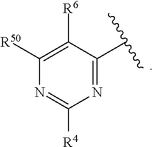

E10.1. The compound of any of E1, E1.1, or E10, or a pharmaceutically acceptable salt thereof, wherein R⁴ is halogen, C₁₋₆alkyl, C₁₋₆haloalkyl, —C₁₋₆alkylene-OR^{4a}, —N(R^{4a})—C₂₋₆alkylene-OR^{4a}, G³, or —N(R^{4a})—G³.
E10.2. The compound of E10.1, or a pharmaceutically acceptable salt thereof, wherein R⁴ is halogen, C₁₋₄alkyl, C₁₋₄fluoroalkyl, —C₁₋₃alkylene-OR^{4a}, —N(R^{4a})—C₂₋₆alkylene-OR^{4a}, G³, or —N(R^{4a})—G³; R^{4a} is hydrogen or C₁₋₄alkyl; and G³ is C₃₋₆cycloalkyl or a 4- to 7-membered heterocyclyl containing 1-2 heteroatoms that are independently O, N, or S, G³ being optionally substituted with 1-4 C₁₋₄alkyl.
E10.3. The compound of E10.2, or a pharmaceutically acceptable salt thereof, wherein R⁴ is chloro, CH₃, CF³, —CH₂—OH, —CH₂—OCH₃, —N(H)—CH₂C(CH₃)₂—OH, cyclopropyl, pyrrolidin-1-yl, morpholino, 4-methylpiperazin-1-yl, —N(H)-tetrahydrofuran-3-yl, or —N(H)— cyclopropyl.
E10.4. The compound of any of E1, E1.1, or E10, or a pharmaceutically acceptable salt thereof, wherein R⁴, R⁵⁰, and R⁶ are independently hydrogen, halogen, C₁₋₄alkyl, —OC₁₋₄alkyl, or C₃₋₈cycloalkyl.

E11. The compound of any of E1 or E10-E10.3, or a pharmaceutically acceptable salt thereof, wherein R⁵⁰ is halogen, C₁₋₄alkyl, —OC₁₋₄alkyl, —C(O)—N(R^{50a})₂, or G³⁰.
E11.1. The compound of E11, or a pharmaceutically acceptable salt thereof, wherein G³⁰ is a 4- to 7-membered heterocyclyl containing a first nitrogen and optionally a second heteroatom that is O, N, or S, the heterocyclyl at G³⁰ being attached to the parent molecular moiety at the first nitrogen.
E11.2. The compound of E11.1, or a pharmaceutically acceptable salt thereof, wherein R⁵⁰ is chloro, CH₃, —OCH₃, —C(O)-morpholino, or pyrrolidin-1-yl.
E11.3. The compound of E11.2, or a pharmaceutically acceptable salt thereof, wherein R⁵⁰ is chloro or CH₃.
E12. The compound of any of E1, E1.1, or E10-E11.3, or a pharmaceutically acceptable salt thereof, wherein R⁶ is C₁₋₄alkyl.
E12.1. The compound of E12, or a pharmaceutically acceptable salt thereof, wherein R⁶ is CH₃.
E13. The compound of any of E1 or E10-E10.3, or a pharmaceutically acceptable salt thereof, wherein R⁵⁰ and R⁶, together with the atom to which each attaches, form a non-aromatic carbocyclic or heterocyclic ring, the heterocyclic ring containing one heteroatom that is O, N, or S and the carbocyclic or heterocyclic ring being optionally substituted with 1-4 C₁₋₄alkyl.
E13.1. The compound of E13, or a pharmaceutically acceptable salt thereof, wherein R⁵⁰ and R⁶, together with the atom to which each attaches, form the non-aromatic 5- to 7-membered carbocyclic ring.
E13.2. The compound of E13.1, or a pharmaceutically acceptable salt thereof, wherein G¹ is

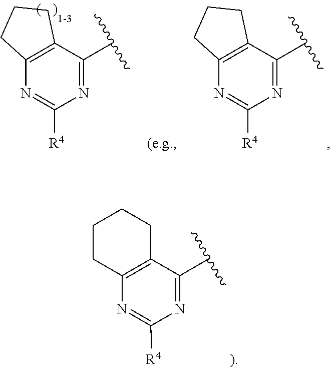

E13.3. The compound of E13, or a pharmaceutically acceptable salt thereof, wherein R⁵⁰ and R⁶, together with the atom to which each attaches, form the non-aromatic 5- to 7-membered heterocyclic ring.
E13.4. The compound of E13.3, or a pharmaceutically acceptable salt thereof, wherein G¹ is

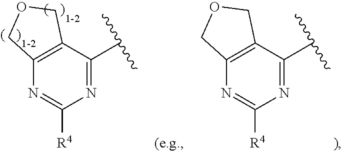

E14. The compound of any of E1-E13.4, or a pharmaceutically acceptable salt thereof, wherein Z¹ is CR¹; and Z³ is CR³.
E15. The compound of any of E1-E14, or a pharmaceutically acceptable salt thereof, wherein R¹ and R³ are hydrogen.
E15.1. The compound of any of E1-E14, or a pharmaceutically acceptable salt thereof, wherein R¹ is CF³ and R³ is hydrogen.
E16. The compound of any of E1-E15.1, or a pharmaceutically acceptable salt thereof, R² is hydrogen, halogen, NO₂, C₁₋₆alkyl, C₁₋₆haloalkyl, —OR^{b}, —NR^{c}, —C₁₋₆alkylene-OH, —C₁₋₆fluoroalkylene-OH, G², or —C₁₋₃alkylene-G².
E16.1. The compound of E16, or a pharmaceutically acceptable salt thereof, wherein R² is hydrogen, C₁₋₆alkyl, C₁₋₆haloalkyl, —OR^{b}, —NR^{b}R^{c}, G², or —C₁₋₃alkylene-G².
E16.2. The compound of E16, or a pharmaceutically acceptable salt thereof, wherein R² is hydrogen, halogen, C₁₋₄alkyl, C₁₋₄fluoroalkyl, —C₁₋₄fluoroalkylene-OH, NH₂, or NO₂.
E16.3. The compound of E16.2, or a pharmaceutically acceptable salt thereof, wherein R² is hydrogen, bromo, CH₃ (e.g., CD3), CHF₂, CF³, CH₂CF₃, —C(OH)(CH₃)(CF³), NH₂, or NO₂.
E16.4. The compound of any of E16-E16.3, or a pharmaceutically acceptable salt thereof, wherein R² is hydrogen.
E17. The compound of any of E1-E16.1, or a pharmaceutically acceptable salt thereof, wherein G² is
   a) a 5- to 6-membered or 9- to 10-membered heteroaryl containing 1-3 heteroatoms independently selected from the group consisting of 0, N, and S;
   b) a phenyl;
   c) a phenyl fused to a 5- to 7-membered heterocycle containing 1-2 oxygen atoms; or
   d) a 4- to 8-membered monocyclic heterocyclyl containing 1-2 heteroatoms independently selected from the group consisting of 0, N, and S;
   wherein G² is optionally substituted with 1-4 substituents independently selected from the group consisting of halogen, cyano, C₁₋₄alkyl, C₁₋₄haloalkyl, —OR^{x}, —N(R^{x})₂, G^{2a}, and —CH₂—G^{2a}; and
   G^{2a} is C₃₋₆cycloalkyl or phenyl and optionally substituted with 1-4 substituents independently selected from the group consisting of halogen, C₁₋₄alkyl, and C₁₋₄haloalkyl.
E18. The compound of E17, or a pharmaceutically acceptable salt thereof, wherein G² is pyrazolyl, thiazolyl, isoxazolyl, pyridinyl, pyridazinyl, pyrimidinyl, benzimidazolyl, imidazo[1,2-a]pyridinyl, indazolyl, phenyl, 1,4-benzodioxin-6-yl, tetrahydrofuranyl, 1,4-dioxanyl, azetidinyl, pyrrolidinyl, or piperidinyl, wherein G² is optionally substituted as defined in E17.
E19. The compound of any of E1-E16.1 or E17-E18, or a pharmaceutically acceptable salt thereof, wherein R² is G².
E19.1. The compound of any of E1-E16.1 or E19, or a pharmaceutically acceptable salt thereof, wherein: G² is:
   a) C₃₋₆cycloalkyl;
   b) phenyl;
   c) a 4- to 10-membered heterocyclyl containing 1-3 heteroatoms independently selected from the group consisting of O, N, and S (e.g., azetidin-1-yl, pyrrolidin-1-yl, piperidin-1-yl, morpholino, piperazin-1-yl, tetrahydrofuran-3-yl, 6,7-dihydropyrazolo[1,5-a]pyrimidin-4(5H)-yl, 6,7-dihydroimidazo[1,2-a]pyrimidin-8(5H)-yl, 3,4-dihydro-1,5-naphthyridin-1(2H)-yl, 2,3-dihydro-1H-pyrido[2,3-b][1,4]oxazin-1-yl); or
   d) a 5- to 6-membered or 9- to 10-membered heteroaryl containing 1-3 heteroatoms independently selected from the group consisting of O, N, and S (e.g., pyrazol-1-yl, pyrazol-3-yl, pyrazol-4-yl, pyrazol-5-yl, imidazol-1-yl, thiazol-5-yl, isothiazol-5-yl, isoxazol-4-yl, pyridin-2-yl, pyridin-3-yl, pyridin-4-yl, pyridazin-4-yl, pyrimidin-5-yl, benzo[d]imidazol-6-yl, imidazo[1,2-a]pyridin-6-yl);
wherein G² is optionally substituted with 1-4 substituents independently selected from the group consisting of halogen, cyano, C₁₋₆alkyl, C₁₋₆haloalkyl, oxo, —OR^{x}, —N(R^{x})₂, —C₁₋₆alkylene-OR^{x}, —C₁₋₆alkylene-N(R^{x})₂, G^{2a}, and —C₁₋₃alkylene-G^{2a}; and G^{2a} is C₃₋₆cycloalkyl(cyclopropyl, cyclobutyl, cyclopentyl, cyclohexyl), phenyl, a 5- to 6-membered heteroaryl containing 1-3 heteroatoms independently selected from the group consisting of O, N, and S (e.g., imidazol-1-yl, pyridin-2-yl, pyridin-3-yl), or a 4- to 8-membered heterocyclyl containing 1-3 heteroatoms independently selected from the group consisting of O, N, and S (e.g., azetidin-1-yl, pyrrolidin-1-yl, morpholino, piperazin-1-yl, 2-oxa-5-azabicyclo[2.2.1]heptan-5-yl, 2-oxa-6-azaspiro[3.3]heptan-6-yl), G²a being optionally substituted with 1-4 substituents independently selected from the group consisting of halogen, C₁₋₄alkyl, C₁₋₄fluoroalkyl, —C₁₋₆alkylene-OH, C₃₋₄cycloalkyl, and —C₁₋₃alkylene-C₃₋₄cycloalkyl.
E19.2. The compound of E19.1, or a pharmaceutically acceptable salt thereof, wherein G² is the optionally substituted C₃₋₆cycloalkyl.
E19.3. The compound of E19.1, or a pharmaceutically acceptable salt thereof, wherein G² is the optionally substituted phenyl.
E19.4. The compound of E19.1, or a pharmaceutically acceptable salt thereof, wherein G² is the optionally substituted 4- to 10-membered heterocyclyl.
E19.5. The compound of E19.1, or a pharmaceutically acceptable salt thereof, wherein G² is the optionally substituted 5- to 6-membered heteroaryl.
E19.6. The compound of E19.1, or a pharmaceutically acceptable salt thereof, wherein G² is the optionally substituted 9- to 10-membered heteroaryl.
19.7. The compound of any of E1-E19.6, or a pharmaceutically acceptable salt thereof, wherein R^{x}, at each occurrence, is independently hydrogen or C₁₋₄alkyl (e.g., methyl).
E19.8. The compound of E19.1, or a pharmaceutically acceptable salt thereof, wherein G² is C₃₋₆cycloalkyl,

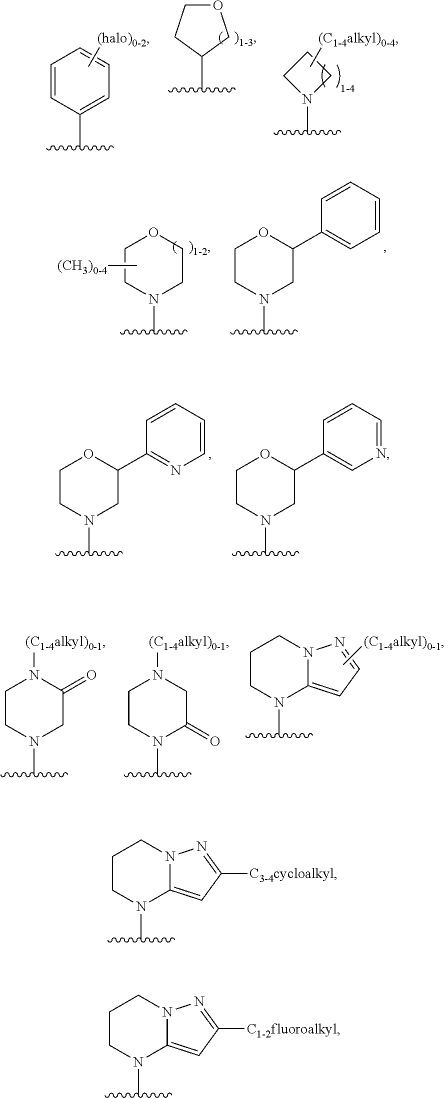

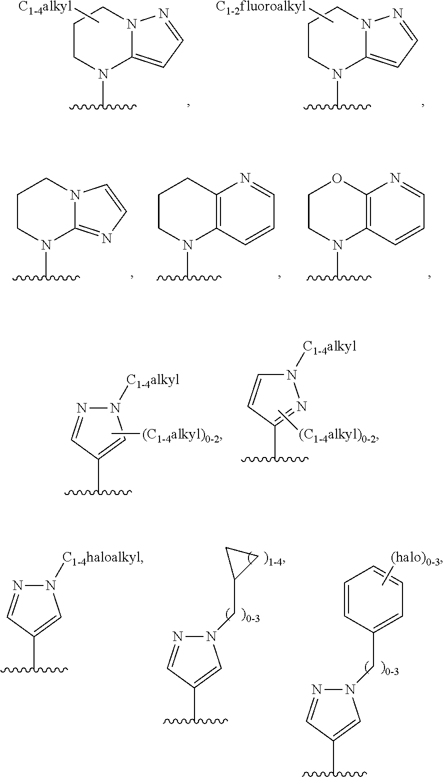

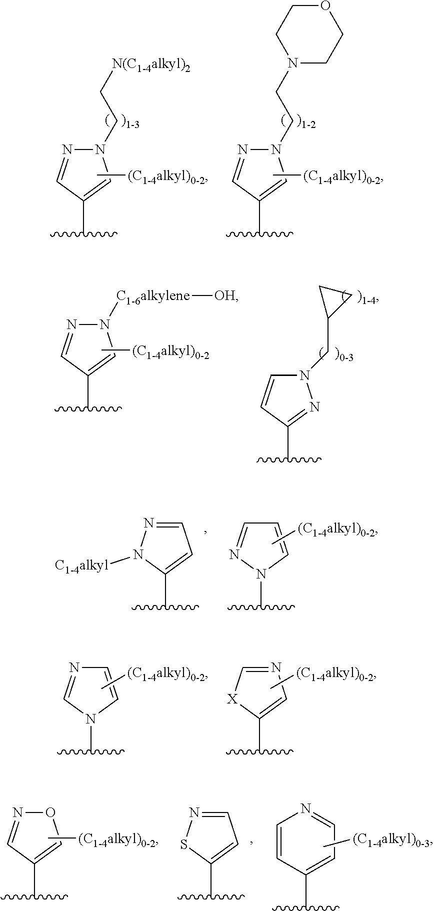

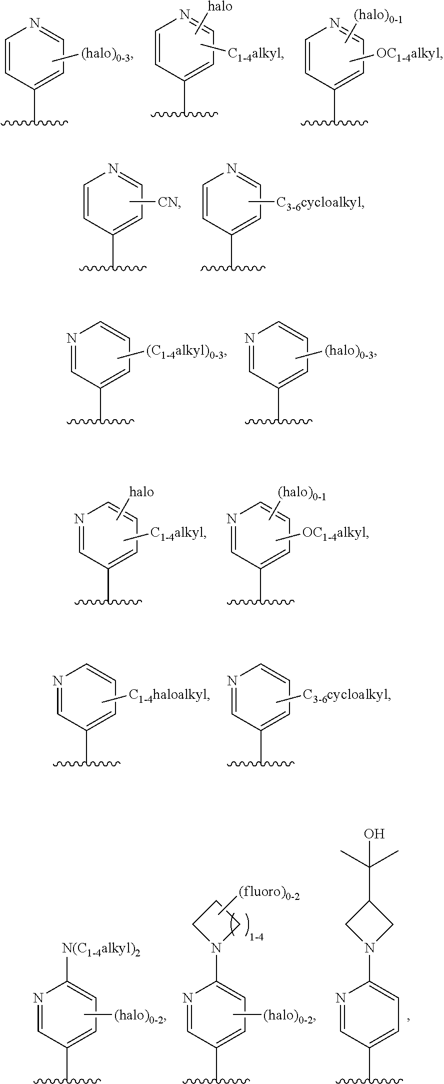

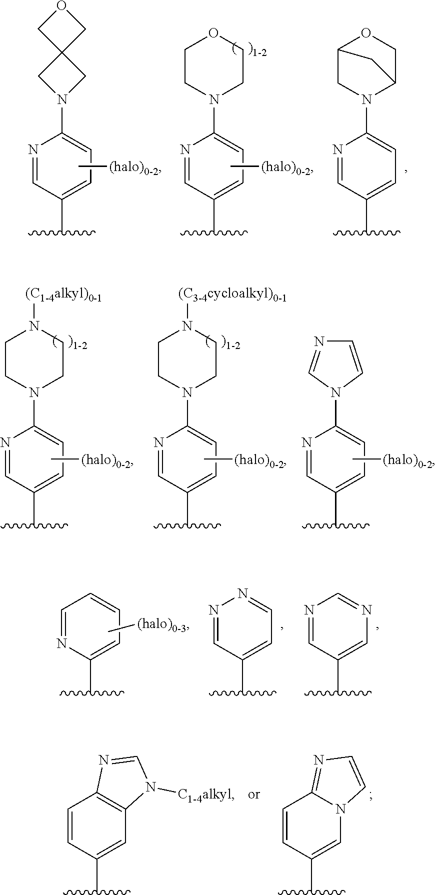

### and X is O, N, or S.

E19.9. The compound of E19.8, or a pharmaceutically acceptable salt thereof, wherein G² is

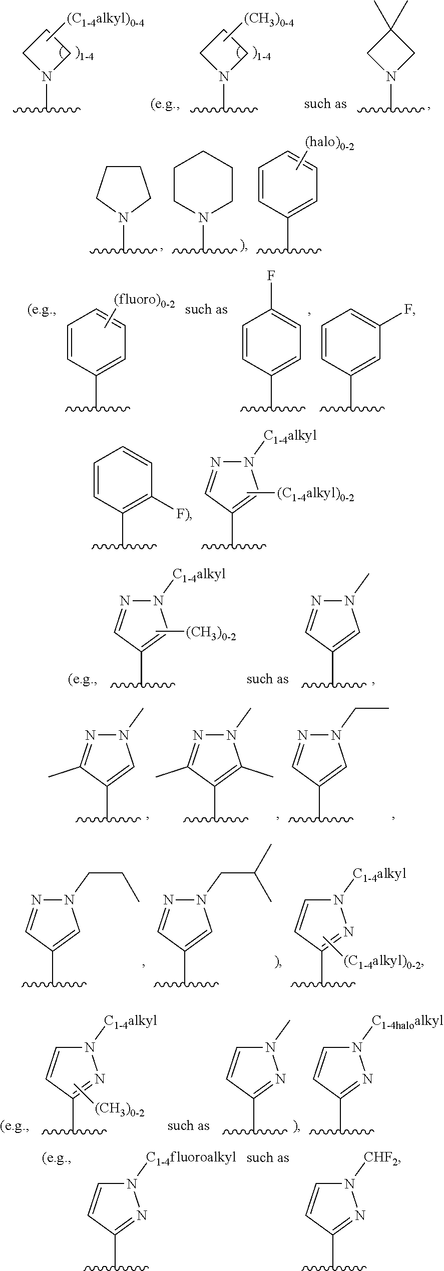

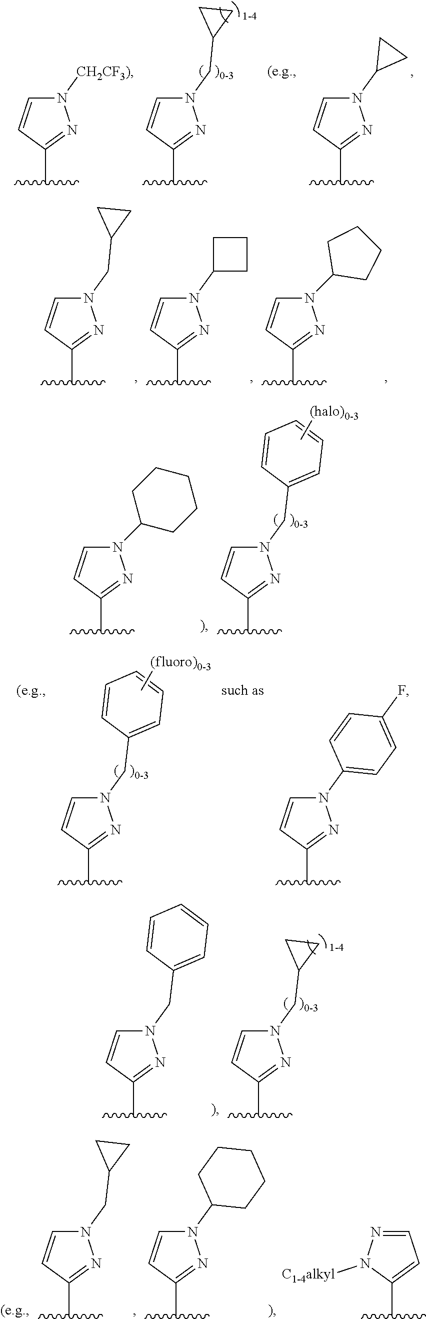

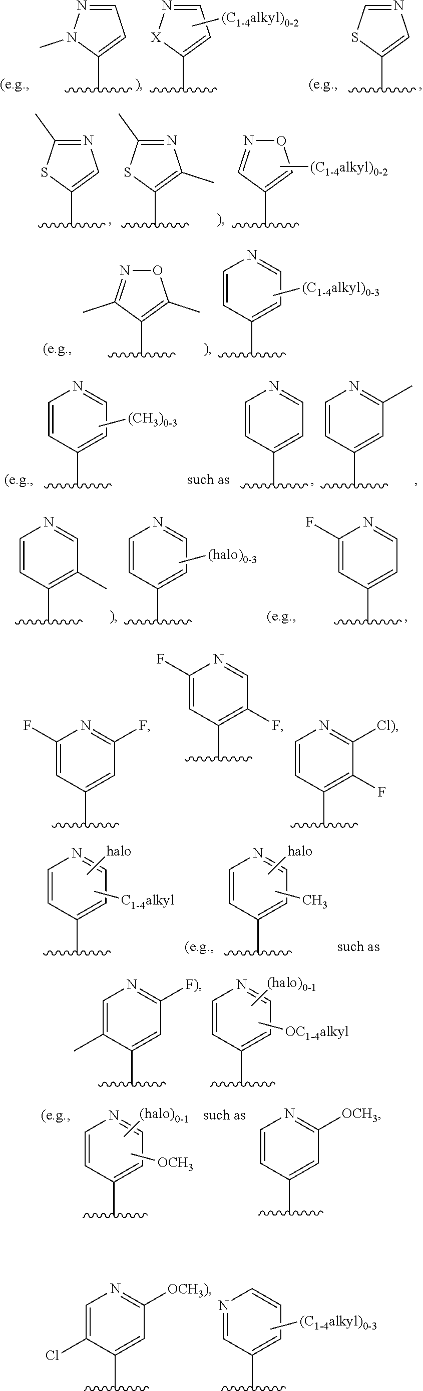

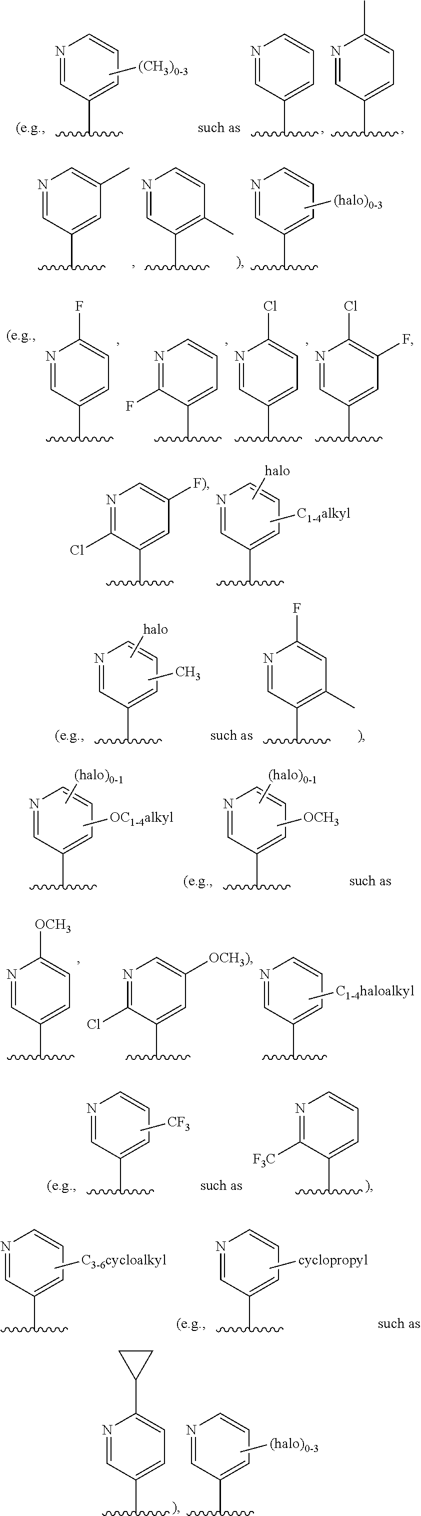

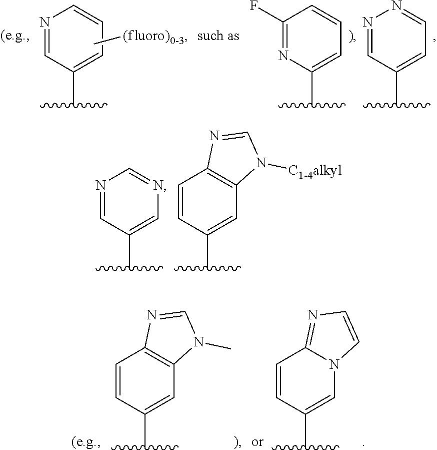

E19.10. The compound of E19.8, or a pharmaceutically acceptable salt thereof, wherein G² is C₃₋₆cycloalkyl (e.g., cyclopropyl, cyclobutyl),

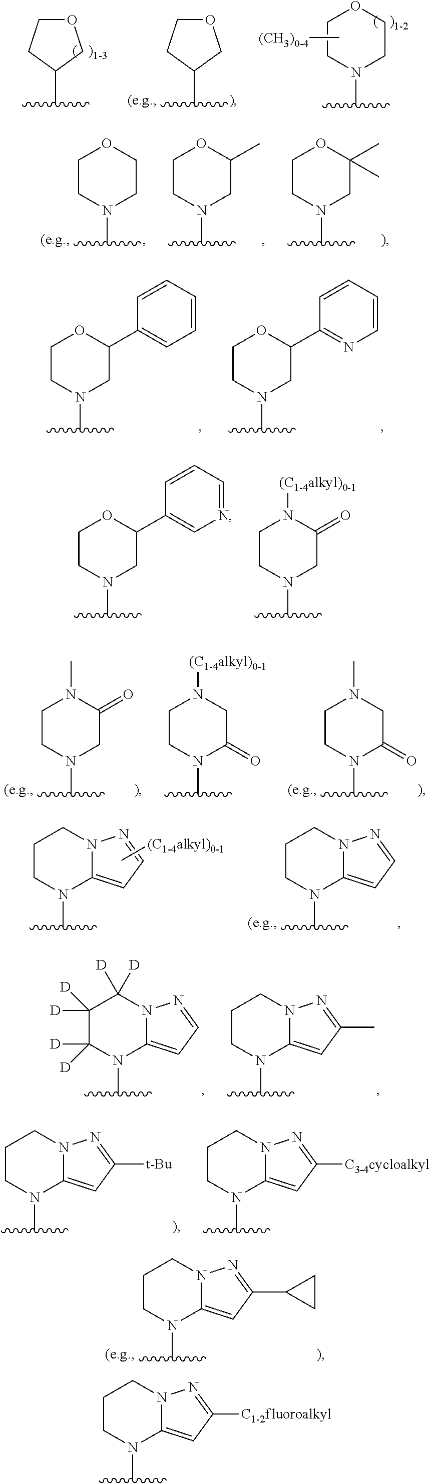

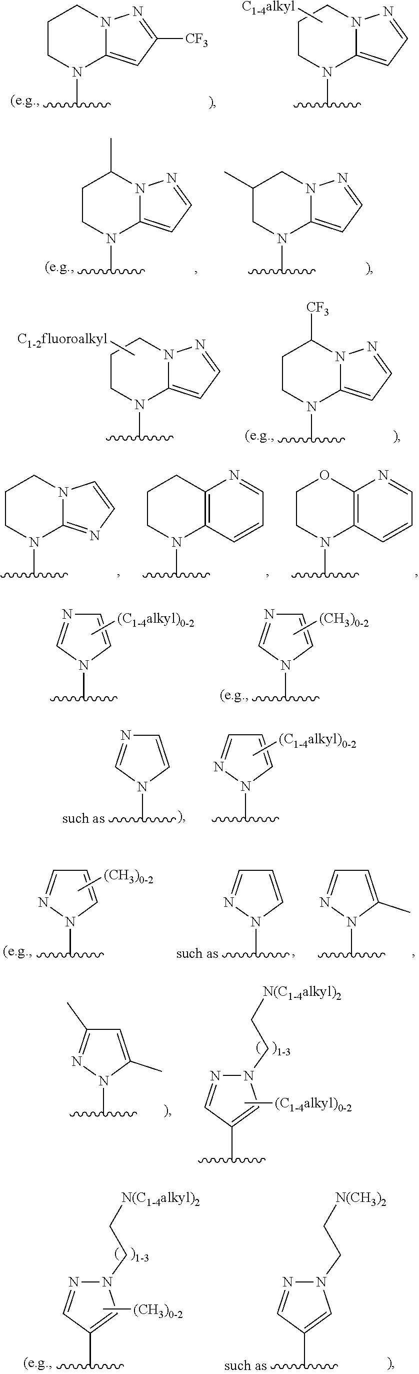

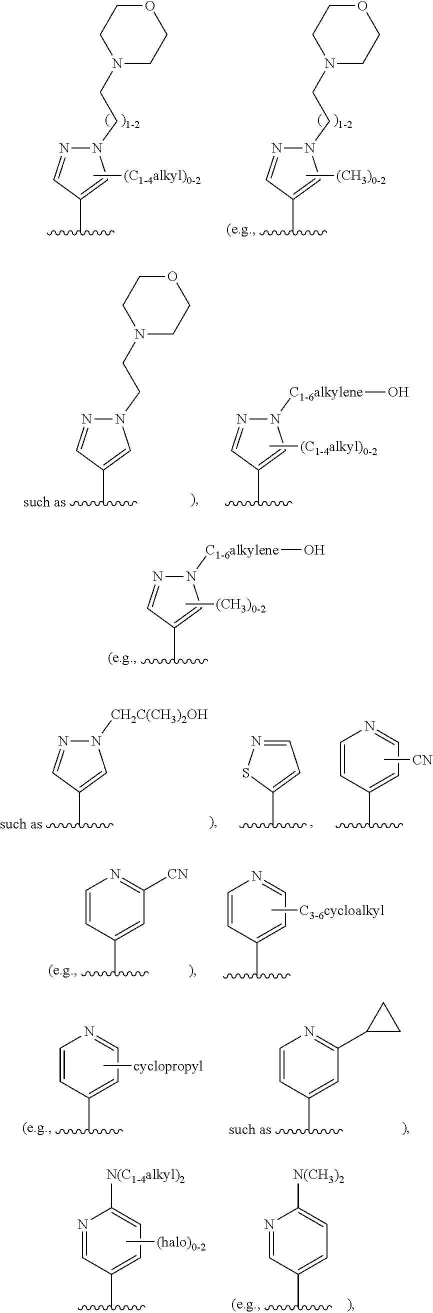

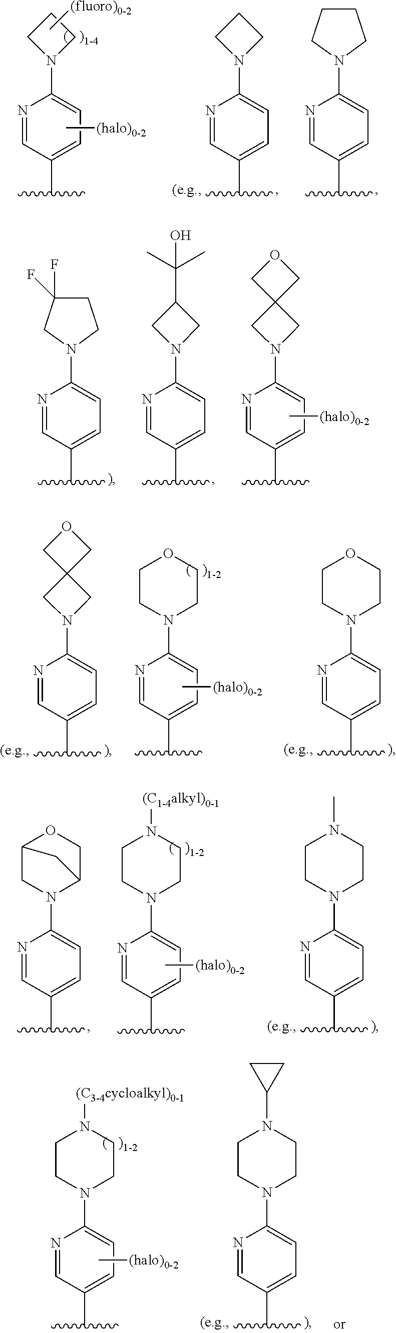

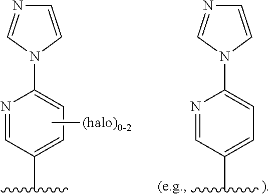

E19.11. The compound of any of E19.1, E19.2, E19.8, or E19.10, or a pharmaceutically acceptable salt thereof, wherein G² is cyclopropyl.
E19.12. The compound of any of E19.1, E19.4, E19.8, or E19.10, or a pharmaceutically acceptable salt thereof, wherein G² is

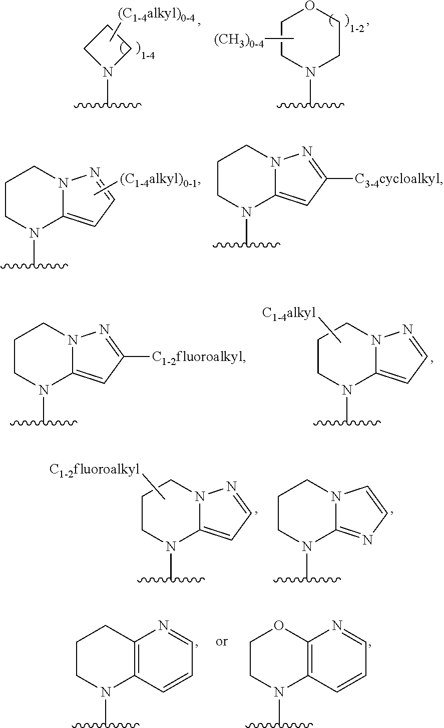

E19.13. The compound of E19.12, or a pharmaceutically acceptable salt thereof, wherein G² is

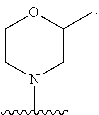 [SMILES: *N1CCOC(C)C1]

E19.14. The compound of E-9.13, or a pharmaceutically acceptable salt thereof, wherein G² is

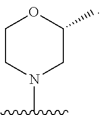 [SMILES: *N1CCO[C@H](C)C1]

E19.15. The compound of E19.13, or a pharmaceutically acceptable salt thereof, wherein G² is

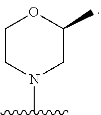 [SMILES: *N1CCO[C@@H](C)C1]

E19.16. The compound of E19.12, or a pharmaceutically acceptable salt thereof, wherein G² is

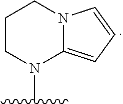 [SMILES: *N1CCCn2cccc21]

E19.17. The compound of any of E19.1, E19.5, or E19.8, or a pharmaceutically acceptable salt thereof, wherein G² is

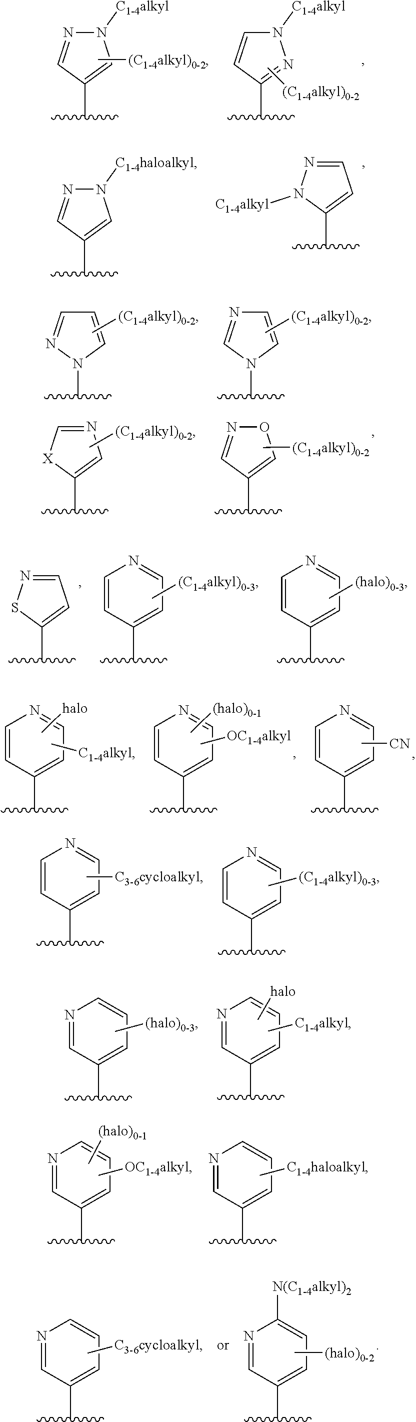

E19.18. The compound of E19.17, or a pharmaceutically acceptable salt thereof, wherein G² is

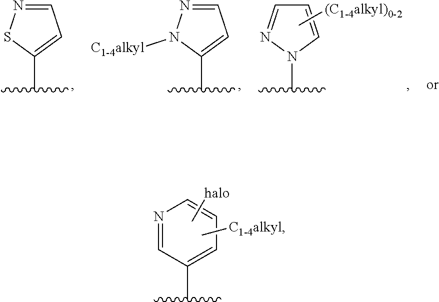

E19.19. The compound of E19.18, or a pharmaceutically acceptable salt thereof, wherein G² is

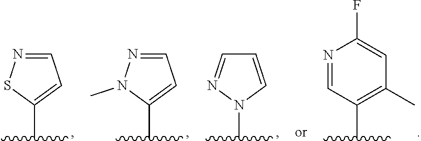

E19.20. The compound of any of E19-E19.7, or a pharmaceutically acceptable salt thereof, wherein G² is optionally substituted with a first substituent independently selected from the group consisting of halogen, cyano, C₁₋₆alkyl, C₁₋₆haloalkyl, oxo, —OR^{x}, —N(R^{x})₂, —C₁₋₆alkylene-OR^{x}, —C₁₋₆alkylene-N(R^{x})₂, G^{2a}, and —C₁₋₃alkylene-G^{2a}; and further optionally substituted with 1-3 substituents independently selected from the group consisting of halogen, C₁₋₄alkyl, C₁₋₄fluoroalkyl, and —OR^{x}.
E19.21. The compound of any of E19-19.7 or E19.20, or a pharmaceutically acceptable salt thereof, wherein G^{2a} is optionally substituted with a first substituent selected from the group consisting of halogen, C₁₋₄alkyl, C₁₋₄fluoroalkyl, —C₁₋₆alkylene-OH, C₃₋₄cycloalkyl, and —C₁₋₃alkylene-C₃₋₄cycloalkyl, and optionally further substituted with 1-3 substituents independently selected from the group consisting of halogen and C₁₋₄alkyl.
E19.22. The compound of E19.1 or E19.3, or a pharmaceutically acceptable salt thereof, wherein G² is

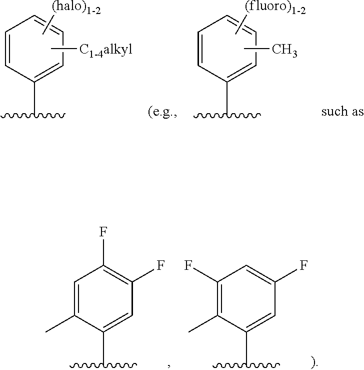

E20. The compound of any of E1-E16.1 or E17-E18, or a pharmaceutically acceptable salt thereof, wherein
   R² is —NR^{b}R^{c};
   R^{b} is —G² or —C₁₋₃alkylene-G²; and
   R^{c} is hydrogen, C₁₋₆alkyl, C₁₋₆haloalkyl, C₃₋₆cycloalkyl, or —C₁₋₃alkylene-C₃₋₆cycloalkyl.
E20.1. The compound of E20, or a pharmaceutically acceptable salt thereof, wherein R^{b} is —G².
E20.2. The compound of E20.1, or a pharmaceutically acceptable salt thereof, wherein G² is phenyl or a 5- to 6-membered heteroaryl containing 1-3 heteroatoms independently selected from the group consisting of O, N, and S (e.g., a 5- to 6-membered heteroaryl containing 1-3 nitrogen atoms such as pyrazol-5-yl, pyridin-2-yl, pyridin-3-yl, pyrimidin-4-yl); wherein G² is optionally substituted with 1-4 substituents independently selected from the group consisting of halogen, cyano, C₁₋₆alkyl, C₁₋₆haloalkyl, —OR^{x}, —N(R^{x})₂, G^{2a}, and —C₁₋₃alkylene-G^{2a}; and G^{2a} is C₃₋₆cycloalkyl.
E20.2a. The compound of E20.2, or a pharmaceutically acceptable salt thereof, wherein G² is the optionally substituted 5- to 6-membered heteroaryl.
E20.3. The compound of any of E20.1-E20.2a, or a pharmaceutically acceptable salt thereof, wherein R^{x}, at each occurrence, is independently hydrogen, C₁₋₄alkyl (e.g., methyl) or C₁₋₄fluoroalkyl (e.g., CF³).
E20.4. The compound of any of E20.1-E20.3, or a pharmaceutically acceptable salt thereof, wherein R² is

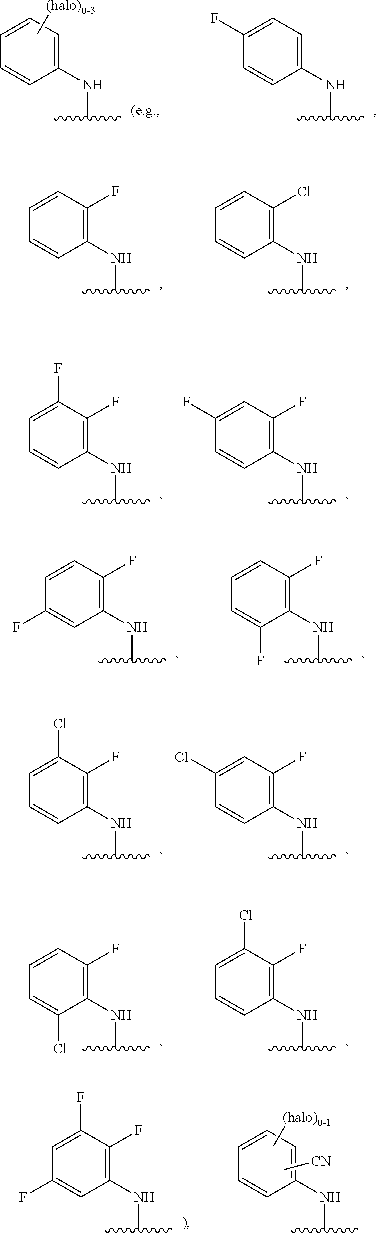

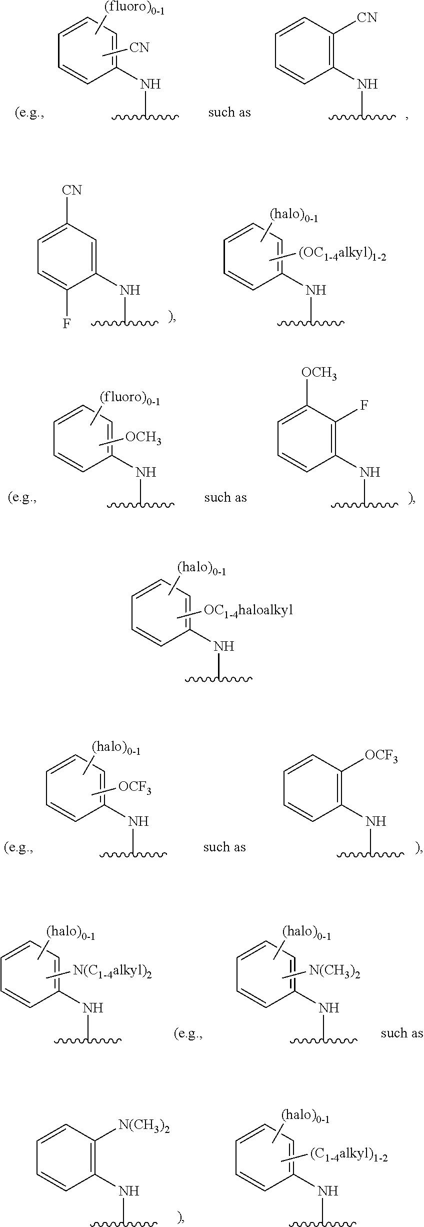

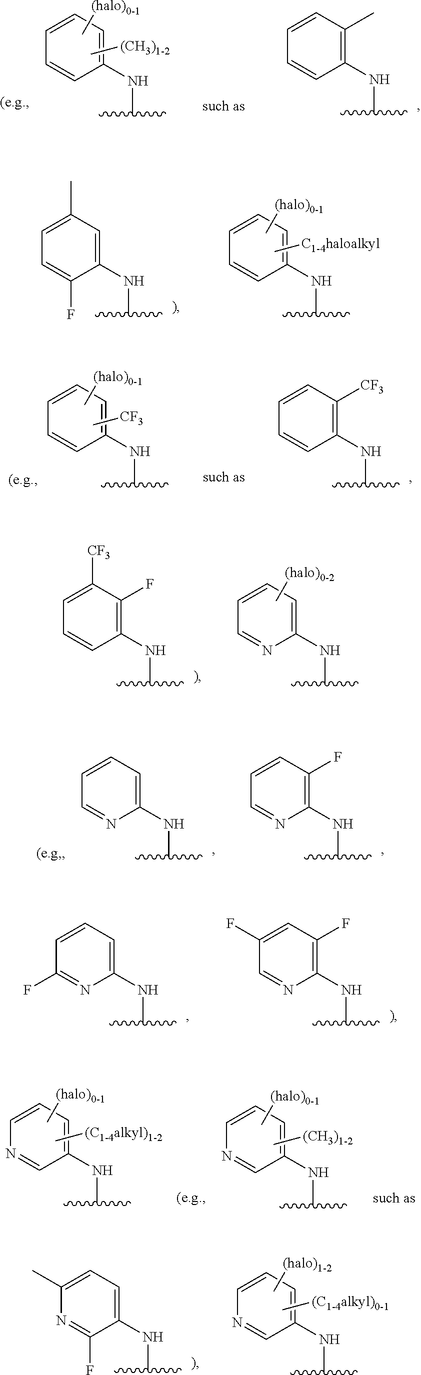

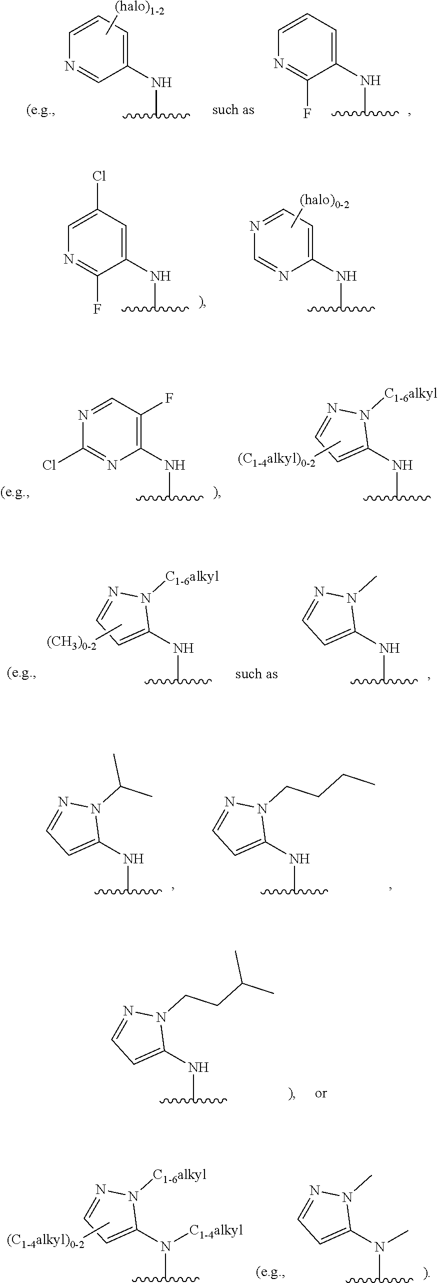

E20.5. The compound of any of E20.1-E20.3, or a pharmaceutically acceptable salt thereof, wherein R² is

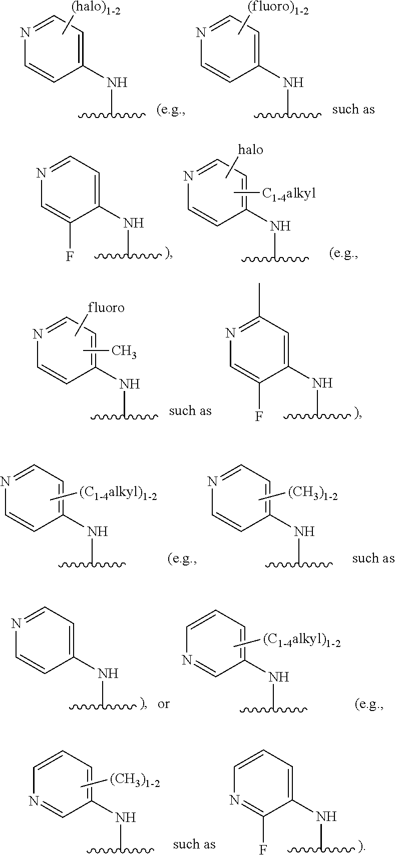

E20.6. The compound of any of E20.1-E20.3, or a pharmaceutically acceptable salt thereof, wherein R² is

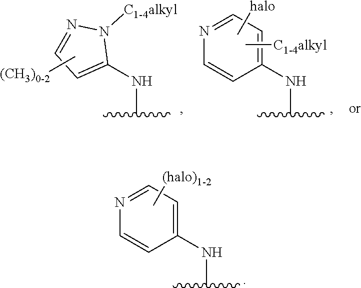

E20.7. The compound of E20.6, or a pharmaceutically acceptable salt thereof, wherein R² is

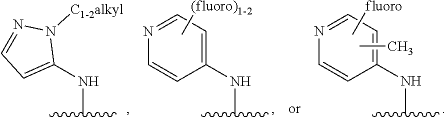

E20.8. The compound of E20.7, or a pharmaceutically acceptable salt thereof, wherein R² is

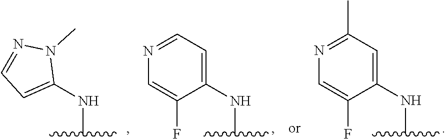

E20.9. The compound of E20, or a pharmaceutically acceptable salt thereof, wherein R^{b} is —C₁₋₃alkylene-G².
E20.10. The compound of E20.9, or a pharmaceutically acceptable salt thereof, wherein G² is phenyl, a 5- to 6-membered heteroaryl containing 1-3 heteroatoms independently selected from the group consisting of O, N, and S (e.g., a 5- to 6-membered heteroaryl containing 1-3 nitrogen atoms such as pyrazol-5-yl, pyridin-2-yl, pyridin-3-yl, pyrimidin-4-yl), or a 4- to 8-membered heterocyclyl containing 1-2 heteroatoms independently selected from the group consisting of O, N, and S (e.g., a 4- to 8-membered heterocyclyl containing 1-2 oxygen atoms such as tetrahydrofuran-2-yl, 1,4-dioxan-2-yl); wherein G² is optionally substituted with 1-4 substituents independently selected from the group consisting of halogen, cyano, C₁₋₆alkyl, C₁₋₆haloalkyl, —OR^{x}, —N(R^{x})₂, G^{2a}, and —C₁₋₃alkylene-G^{2a}; and G^{2a} is C₃₋₆cycloalkyl.
E20.11. The compound of any of E20.9-E20.10, or a pharmaceutically acceptable salt thereof, wherein R^{x}, at each occurrence, is independently hydrogen, C₁₋₄alkyl (e.g., methyl) or C₁₋₄fluoroalkyl (e.g., CF³).
E20.12. The compound of any of E20.9-E20.11, or a pharmaceutically acceptable salt thereof, wherein R² is

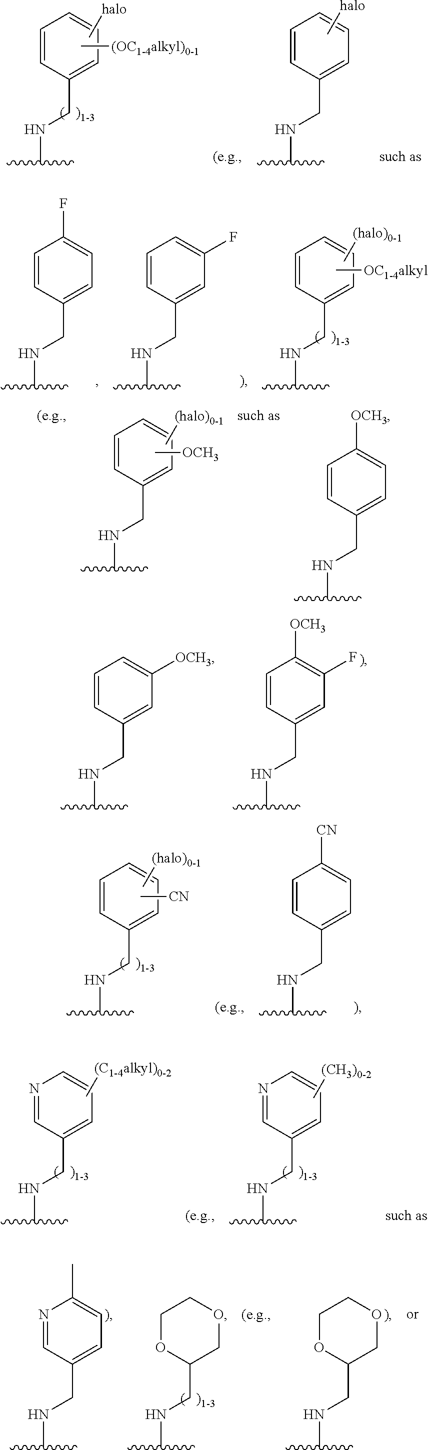

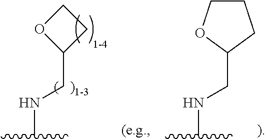

E20.13. The compound of any of E20-E20.3 or E20.9-E20.11, or a pharmaceutically acceptable salt thereof, wherein R^{c} is hydrogen or methyl.
E20.14. The compound of any of E20-E20.3, E20.9-E20.11, or E20.13, or a pharmaceutically acceptable salt thereof, wherein G² is optionally substituted with a first substituent independently selected from the group consisting of halogen, cyano, C₁₋₆alkyl, C₁₋₆haloalkyl, —OR^{x}, —N(R^{x})₂, G^{2a}, and —C₁₋₃alkylene-G^{2a}, and optionally further substituted with 1-3 substituents independently selected from the group consisting of halogen and C₁₋₄alkyl; and G²a is C₃₋₆cycloalkyl.
E21. The compound of any of E1-E16.1 or E17-E18, or a pharmaceutically acceptable salt thereof, wherein R² is —C₁₋₃alkylene-G² (e.g., —CH₂—G²).
E21.1. The compound of E21, or a pharmaceutically acceptable salt thereof, wherein G² is phenyl optionally substituted with 1-4 substituents independently selected from the group consisting of halogen, cyano, C₁₋₄alkyl, C₁₋₄haloalkyl, —OR^{x}, and —N(R^{x})₂.
E21.2. The compound of E21.1, or a pharmaceutically acceptable salt thereof, wherein R² is or

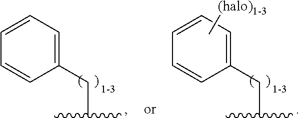

E21.3. The compound of E21.2, or a pharmaceutically acceptable salt thereof, wherein R² is

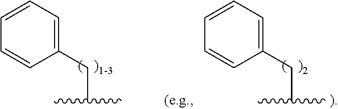

E21.4. The compound of E21.2, or a pharmaceutically acceptable salt thereof, wherein R² is

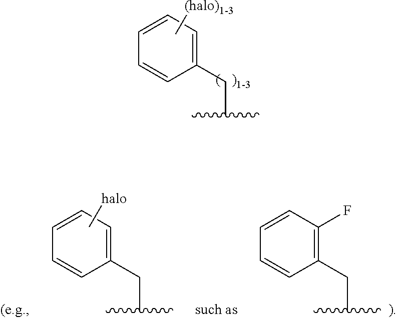

E22. The compound of any of E1-E16.1 or E17-E18, or a pharmaceutically acceptable salt thereof, wherein R² is —OR^{b}; and R^{b} is G².
E22.1. The compound of E22, or a pharmaceutically acceptable salt thereof, wherein G² is phenyl, a phenyl fused to a 5- to 7-membered heterocycle containing 1-2 oxygen atoms (e.g., 2,3-dihydrobenzo[b][1,4]dioxin-6-yl), a 5- to 6-membered heteroaryl, or a 9- to 10-membered heteroaryl, each heteroaryl containing 1-3 heteroatoms independently selected from the group consisting of O, N, and S (e.g., pyridin-3-yl, indazol-5-yl), G² being optionally substituted with 1-4 substituents independently selected from the group consisting of halogen, cyano, C₁₋₄alkyl, C₁₋₄haloalkyl, —OR^{x}, and —N(R^{x})₂.
E22.2. The compound of E22 or E22.1, or a pharmaceutically acceptable salt thereof, wherein R^{x}, at each occurrence, is independently hydrogen, C₁₋₄alkyl (e.g., methyl) or C₁₋₄fluoroalkyl (e.g., CF³).
E22.3. The compound of E22.2, or a pharmaceutically acceptable salt thereof, wherein R² is

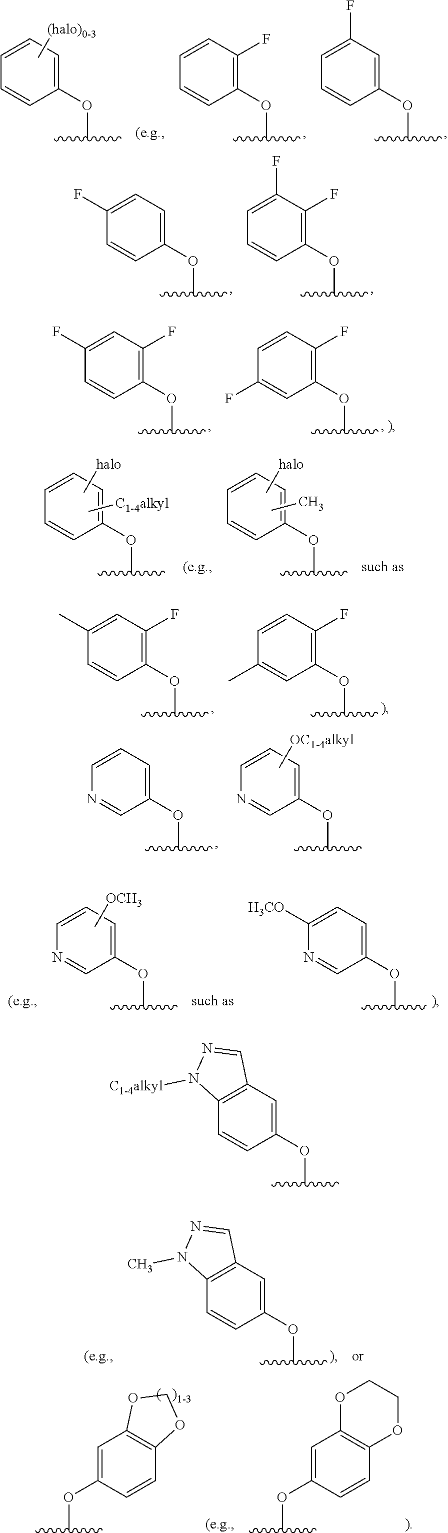

E23. The compound of any of E1-E16.1 or E17-E18, or a pharmaceutically acceptable salt thereof, wherein R² is —OR^{b}; and R^{b} is —C₂₋₄alkylene-O—C₁₋₄alkyl.
E23.1. The compound of E23, or a pharmaceutically acceptable salt thereof, wherein R^{b} is —(CH₂)₂—O—C₁₋₄alkyl.
E23.2. The compound of E23, or a pharmaceutically acceptable salt thereof, wherein R^{b} is —C₂₋₄alkylene-O—CH₃.
E23.3. The compound of any of E23-E23.2, or a pharmaceutically acceptable salt thereof, wherein R^{b} is —(CH₂)₂—O—CH₃.
E24. The compound of any of E1-E16.1 or E17-E18, or a pharmaceutically acceptable salt thereof, wherein R² is —OR^{b}; and R^{b} is —C₂₋₄alkylene-O—G².
E24.1. The compound of E24, or a pharmaceutically acceptable salt thereof, wherein R^{b} is —(CH₂)₂—O—G² or —(CH₂)₃—O—G².
E24.2. The compound of E24 or E24.1, or a pharmaceutically acceptable salt thereof, wherein G² is phenyl or C₃₋₆cycloalkyl, the phenyl and cycloalkyl being optionally substituted with 1-4 substituents independently selected from the group consisting of halogen and C₁₋₄alkyl.
E24.3. The compound of E24.2, or a pharmaceutically acceptable salt thereof, wherein G² is phenyl, 2-fluorophenyl, 4-fluorophenyl, 2-methylphenyl, 4-methylphenyl, or cyclopentyl.
E25. The compound of any of E1-E16.1, or a pharmaceutically acceptable salt thereof, wherein R² is hydrogen, halogen, C₁₋₆alkyl, C₁₋₆haloalkyl,

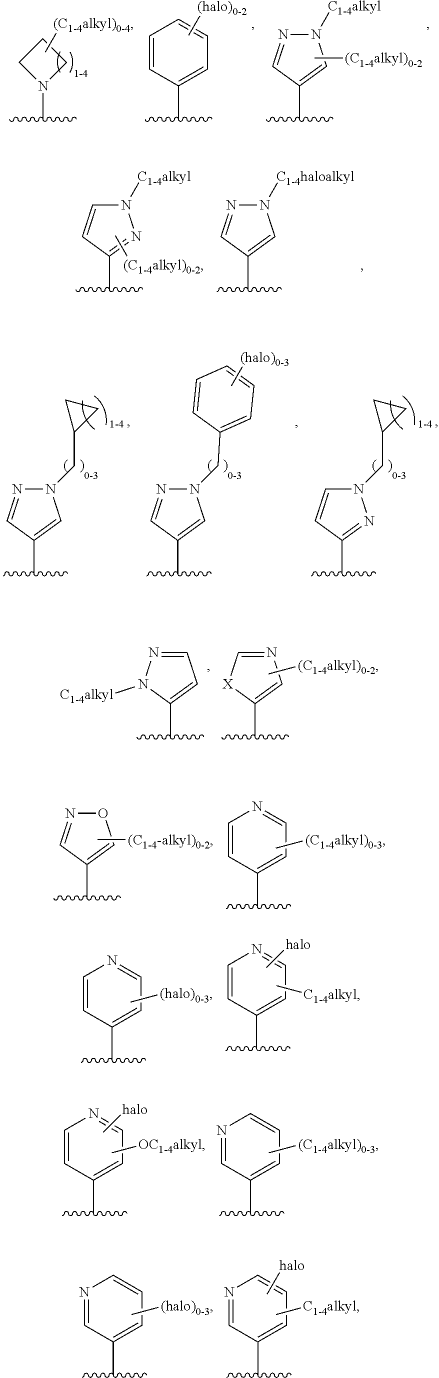

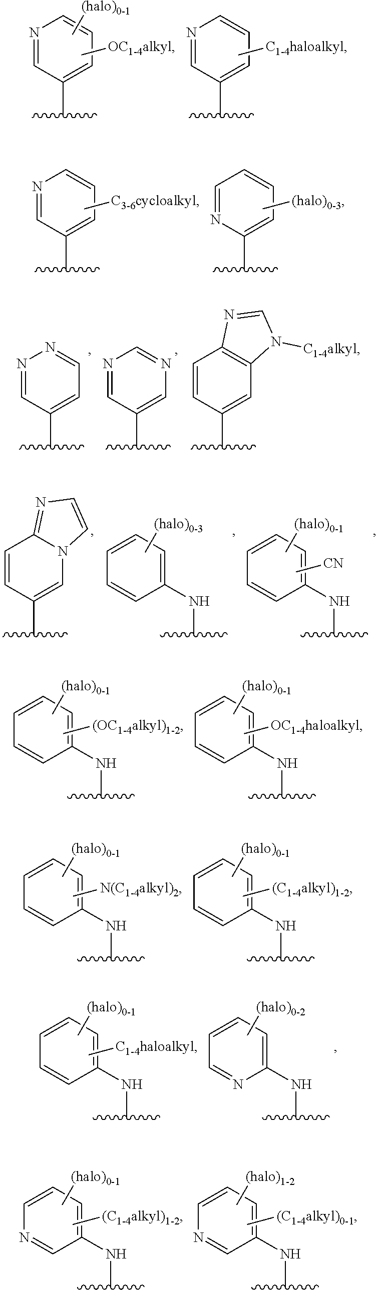

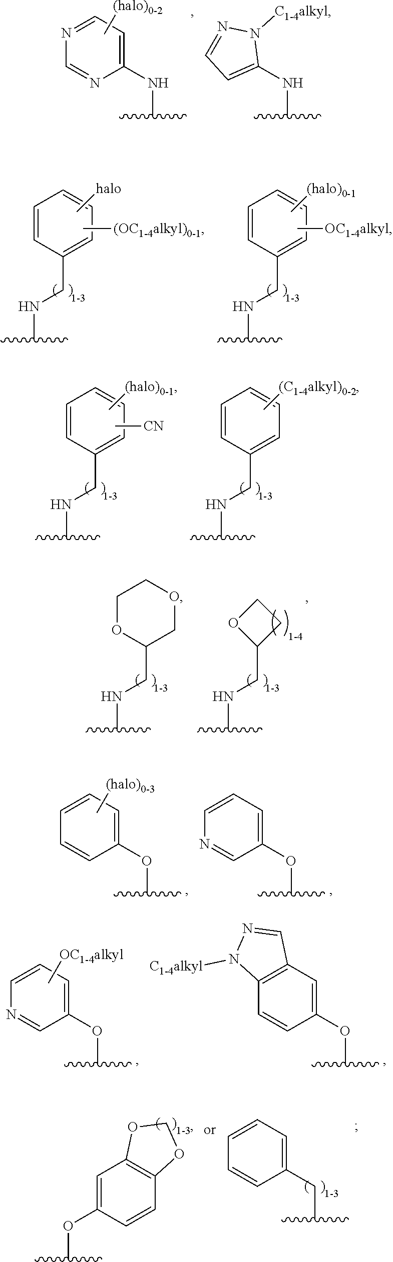

### and X is O, N, or S.

E26. The compound of any of E1-E25, or a pharmaceutically acceptable salt thereof, wherein R⁸ is cyano, —C(O)OR^{8b}, —C(O)R^{8b}, or —C(O)NR^{8b}R^{8c}.
E27. The compound of E26, or a pharmaceutically acceptable salt thereof, wherein
   R⁸ is —C(O)R^{b};
   R^{8b} is G⁴; and
   G⁴ is a 4- to 10-membered heterocyclyl containing 1-3 heteroatoms independently selected from the group consisting of O, N, and S, wherein G⁴ is optionally substituted as defined in E1 or E1.1.
E27.1. The compound of any of E1-E27, or a pharmaceutically acceptable salt thereof, wherein G⁴ is a 4- to 8-membered monocyclic heterocyclyl containing 1-2 heteroatoms, the first heteroatom being a nitrogen and the second heteroatom being independently selected from the group consisting of O, N, and S, wherein G⁴ is attached to the parent molecular moiety at the first heteroatom and optionally substituted with a first substituent selected from the group consisting of halogen, cyano, C₁₋₄alkyl, C₁₋₄fluoroalkyl, —C₁₋₄alkylene-OH, oxo, —OR⁸⁰, —N(R⁸⁰)₂, G^{4a}, and —C₁₋₃alkylene-G^{4a}, G⁴ being further optionally substituted with 1-3 substituents independently selected from the group consisting of halogen, C₁₋₄alkyl, C₁₋₄ fluoroalkyl, —C₁₋₄alkylene-OH, and —OR⁸⁰; and G^{4a} is a phenyl, a 5- to 6-membered heteroaryl containing 1-3 heteroatoms that are independently O, N, or S, a 4- to 6-membered heterocyclyl containing 1-2 heteroatoms that are independently O, N, or S, or a C₃₋₆cycloalkyl.
E27.2. The compound of E27.1, or a pharmaceutically acceptable salt thereof, wherein the optionally substituted 4- to 8-membered monocyclic heterocyclyl containing 1-2 heteroatoms at G⁴ is optionally substituted azetidin-1-yl, pyrrolidin-1-yl, piperazin-1-yl, or morpholino.
E27.3. The compound of any of E1-E27.2, or a pharmaceutically acceptable salt thereof, wherein R⁸⁰, at each occurrence, is independently hydrogen, C₁₋₄alkyl, C₁₋₄fluoroalkyl, C₃₋₄cycloalkyl, or —C₁₋₃alkylene-C₃₋₄cycloalkyl.
E27.4. The compound of any of E1-E27.3, or a pharmaceutically acceptable salt thereof, wherein G⁴ is

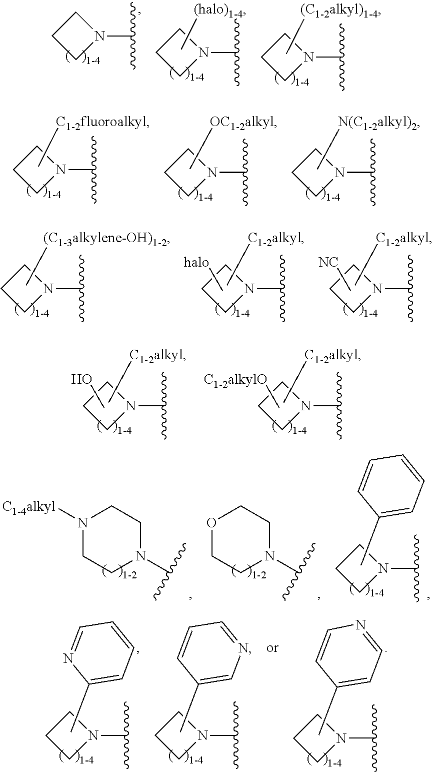

E27.5. The compound of E27.4, or a pharmaceutically acceptable salt thereof, wherein G⁴ is

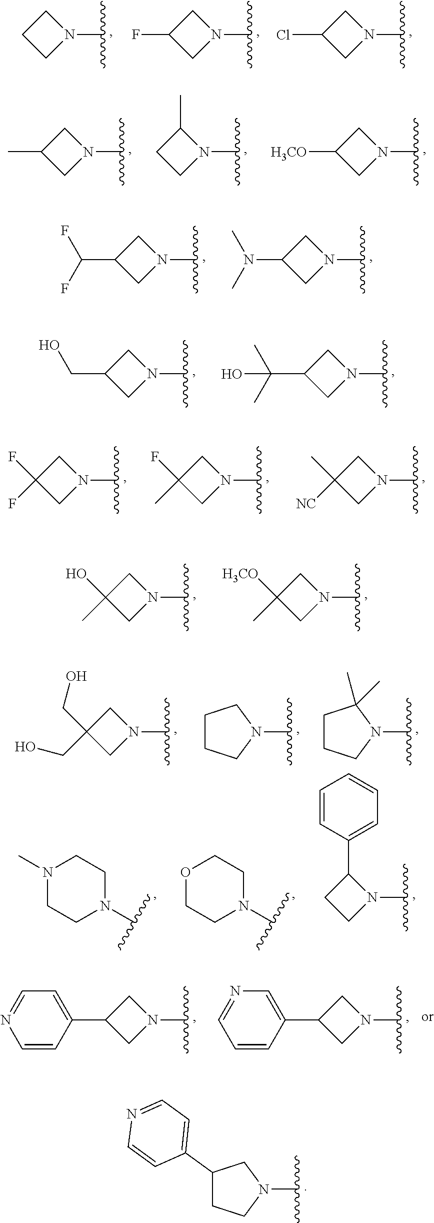

E27.6. The compound of E27.4, or a pharmaceutically acceptable salt thereof, wherein G⁴ is

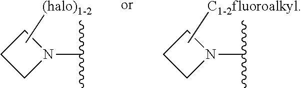

E27.7. The compound of E27.6, or a pharmaceutically acceptable salt thereof, wherein G⁴ is

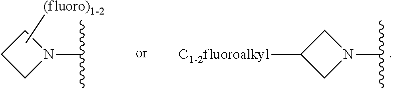

E27.8. The compound of E27.7, or a pharmaceutically acceptable salt thereof, wherein G⁴ is

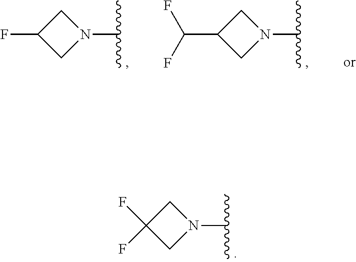 [SMILES: *N1CC(F)C1 ; *N1CC(C(F)F)C1 ; *N1CC(F)(F)C1]

E27.9. The compound of any of E1-E27, or a pharmaceutically acceptable salt thereof, wherein G⁴ is a 7- to 12-membered heterocyclyl containing 1-3 heteroatoms, the first heteroatom being a nitrogen and the second and third heteroatoms being independently selected from the group consisting of O, N, and S, wherein G⁴ is attached to the parent molecular moiety at the first heteroatom and optionally substituted with 1-4 substituents independently selected from the group consisting of halogen and C₁₋₄alkyl.
E27.10. The compound of E27.9, or a pharmaceutically acceptable salt thereof, wherein the optionally substituted 7- to 12-membered heterocyclyl containing 1-3 heteroatoms at G⁴ is optionally substituted 3,4-dihydro-2,6-naphthyridin-2(1H)-yl, 3,4-dihydro-2,7-naphthyridin-2(1H)-yl, 5,8-dihydro-1,7-naphthyridin-7(6H)-yl, 6,7-dihydropyrazolo[1,5-a]pyrimidin-4(5H)-yl, 3-azabicyclo[3.1.0]hexan-3-yl, 2-azaspiro[3.3]heptan-2-yl, 2-oxa-6-azaspiro[3.3]heptan-6-yl, 1,3-dihydro-2H-pyrrolo[3,4-c]pyridin-2-yl, or 5,7-dihydro-6H-pyrrolo[3,4-b]pyridin-6-yl.
E27.11. The compound of E27.9 or E27.10, or a pharmaceutically acceptable salt thereof, wherein G⁴ is

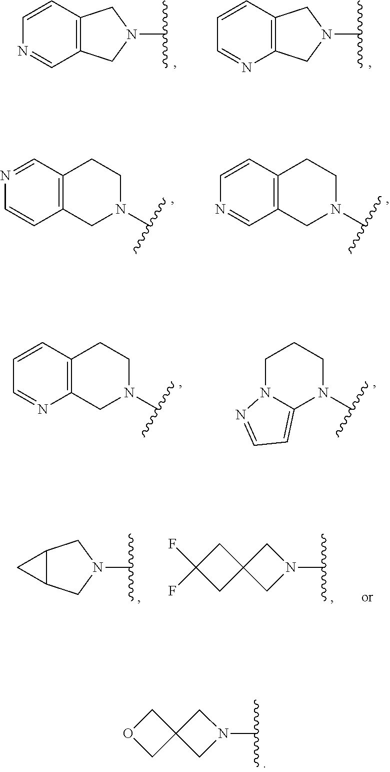

E27.12. The compound of E27.11, or a pharmaceutically acceptable salt thereof, wherein G⁴ is

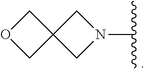 [SMILES: *N1CC2(COC2)C1]

E28. The compound of E26, or a pharmaceutically acceptable salt thereof, wherein R⁸ is —C(O)NR^{8b}R^{8c}.
E28.1. The compound of any of E1-E26 or E28, or a pharmaceutically acceptable salt thereof, wherein R^{8b} is C₁₋₆alkyl, G⁴, or —C₁₋₃alkylene-G⁴; and G⁴ is
   a) a 5- to 6-membered or 9- to 10-membered heteroaryl containing 1-3 heteroatoms independently selected from the group consisting of O, N, and S (e.g., pyridin-4-yl, pyridin-3-yl, pyridin-2-yl, pyrimidin-2-yl, thiazol-2-yl, thiazol-4-yl, thiazol-5-yl, pyrazol-5-yl, isoxazol-5-yl, pyrrol-2-yl, quinolin-3-yl, quinolin-4-yl, quinolin-6-yl, quinolin-8-yl, quinoxalin-6-yl, benzothiophen-3-yl, benzothiazol-2-yl, [1,2,4]triazolo[1,5-a]pyridin-6-yl, pyrazolo[1,5-a]pyridin-3-yl, imidazo[1,2-a]pyridin-2-yl, benzotriazol-5-yl, 6,7-dihydro-5H-cyclopenta[b]pyridin-3-yl, benzofuran-2-yl);
   b) phenyl;
   c) a phenyl fused to a 5- to 7-membered heterocycle containing 1-2 heteroatoms independently selected from the group consisting of O, N, and S (e.g., 1,3-dihydroisobenzofuran-5-yl, indolin-5-yl, 2,3-dihydro-1H-benzo[d]imidazol-5-yl, 2,3-dihydrobenzothiophen-5-yl);
   d) a 4- to 10-membered heterocyclyl containing 1-3 heteroatoms independently selected from the group consisting of O, N, and S (e.g., tetrahydropyran-4-yl, tetrahydropyran-2-yl, 2-oxaspiro[3.3]heptan-6-yl, azetidin-3-yl); or
   e) a C₃₋₈cycloalkyl (e.g., cyclopropyl, cyclobutyl, cyclopentyl, cyclohexyl, bicyclo[1.1.1]pentan-1-yl);
   wherein G⁴ is optionally substituted as defined in E1 or E1.1.
E28.2. The compound of any of E1-E26 or E28, or a pharmaceutically acceptable salt thereof, wherein G⁴ is a 5- to 6-membered carbocyclyl fused to a 6-membered arene or heteroarene that contains 1-2 nitrogen atoms (e.g., 5,6,7,8-tetrahydroquinolin-6-yl).
E28.3. The compound of any of E1-E26 or E28, or a pharmaceutically acceptable salt thereof, wherein R^{8b} is hydrogen or C₁₋₆alkyl (e.g., methyl, isopropyl, or isobutyl).
E28.4. The compound of E28.1 or E28.2, or a pharmaceutically acceptable salt thereof, wherein R^{8b} is G⁴.
E28.5. The compound of E28.1 or E28.2, or a pharmaceutically acceptable salt thereof, wherein R^{8b} is —C₁₋₃alkylene-G⁴ (e.g., —CH₂—G⁴, —(CH₂)₂-alkylene-G⁴).
E28.6. The compound of E28.4 or E28.5, or a pharmaceutically acceptable salt thereof, wherein G⁴ is the optionally substituted 5- to 6-membered heteroaryl.
E28.7. The compound of E28.4 or E28.5, or a pharmaceutically acceptable salt thereof, wherein G⁴ is the optionally substituted 9- to 10-membered heteroaryl.
E28.8. The compound of E28.4 or E28.5, or a pharmaceutically acceptable salt thereof, wherein G⁴ is the optionally substituted phenyl.
E28.9. The compound of E28.4 or E28.5, or a pharmaceutically acceptable salt thereof, wherein G⁴ is the optionally substituted phenyl fused to a 5- to 7-membered heterocycle.
E28.10. The compound of E28.4 or E28.5, or a pharmaceutically acceptable salt thereof, wherein G⁴ is the optionally substituted 4- to 10-membered heterocyclyl.
E28.11. The compound of E28.4 or E28.5, or a pharmaceutically acceptable salt thereof, wherein G⁴ is the optionally substituted C₃₋₈cycloalkyl.
E28.12. The compound of E28.4 or E28.5, or a pharmaceutically acceptable salt thereof, wherein G⁴ is the optionally substituted 5- to 6-membered carbocyclyl fused to a 6-membered arene or heteroarene.
E28.13. The compound of any of E1-E26 or E28-E28.12, or a pharmaceutically acceptable salt thereof, wherein G⁴ is optionally substituted with 1-4 substituents independently selected from the group consisting of halogen (e.g., fluoro, chloro, bromo), cyano, C₁₋₄alkyl (e.g., methyl), C₁₋₄fluoroalkyl (e.g., CF³, CH₂CF₃), OH, oxo, —OC₁₋₄alkyl (e.g., —OCH₃), —OC₁₋₄ fluoroalkyl, —C(O)OC₁₋₄alkyl (e.g., —C(O)OCH₃), —C(O)NHC₁₋₄alkyl (e.g., —C(O)NHCH₃), —C(O)N(C₁₋₄alkyl)₂, —SO₂C₁₋₄alkyl (e.g., —SO₂CH₃), G⁴a (e.g., pyrazolyl, pyrazol-1-yl, imidazolyl, imidazol-1-yl, morpholino, pyridin-2-yl, pyridin-3-yl), and —C₁₋₃alkylene-G^{4a}.
E28.14. The compound of E28.13, or a pharmaceutically acceptable salt thereof, wherein G⁴ is optionally substituted with a first substituent selected from the group consisting of halogen (e.g., fluoro, chloro, bromo), cyano, C₁₋₄alkyl (e.g., methyl), C₁₋₄fluoroalkyl (e.g., CF³, CH₂CF₃), OH, oxo, —OC₁₋₄alkyl (e.g., —OCH₃), —OC₁₋₄fluoroalkyl, —C(O)OC₁₋₄alkyl (e.g., —C(O)OCH₃), —C(O)NHC₁₋₄alkyl (e.g., —C(O)NHCH₃), —C(O)N(C₁₋₄alkyl)₂, —SO₂C₁₋₄alkyl (e.g., —SO₂CH₃), G⁴a (e.g., pyrazolyl, pyrazol-1-yl, imidazolyl, imidazol-1-yl, morpholino, pyridin-2-yl, pyridin-3-yl), and optionally further substituted with 1-3 substituents independently selected from the group consisting of halogen, C₁₋₄alkyl, and oxo.
E28.15. The compound of any of E1-E26, E28.4, E28.11, or E28.13-E28.14, or a pharmaceutically acceptable salt thereof, wherein R^{8b} is C₃₋₆cycloalkyl optionally substituted with 1-2 halogen or cyano.
E28.16. The compound of any of E1-E26, E28.4, E28.11, or E28.13-E28.14, or a pharmaceutically acceptable salt thereof, wherein R^{8b} is

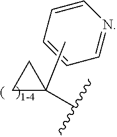

E28.17. The compound of any of E1-E26, E28.4, E28.11, or E28.13-E28.14, or a pharmaceutically acceptable salt thereof, wherein R^{8b} is cyclopropyl, 1-cyanocyclopropyl, 1-(pyridin-2-yl)cyclopropyl, 1-(pyridin-3-yl)cyclopropyl, cyclobutyl, 3,3-difluorocyclobutyl, cyclopentyl, cyclohexyl, bicyclo[1.1.1]pentan-1-yl, or 3-fluorobicyclo[1.1.1]pentan-1-yl.
E28.18. The compound of any of E1-E26, E28.4, E28.6, E28.10, or E28.13-E28.14, or a pharmaceutically acceptable salt thereof, wherein R^{8b} is

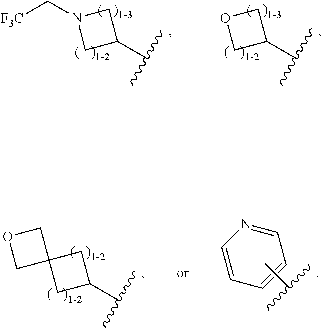

E28.19. The compound of E28.18, or a pharmaceutically acceptable salt thereof, wherein R^{8b} is

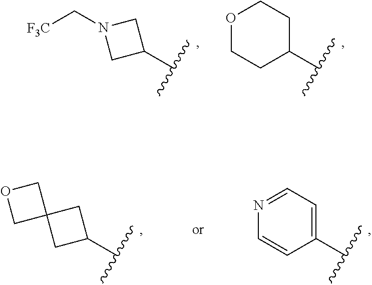 [SMILES: *C1CN(CC(F)(F)F)C1 ; *C1CCOCC1 ; *C1CC2(COC2)C1 ; *c1ccncc1]

E28.20. The compound of any of E1-E26, E28.5, E28.10, or E28.13-E28.14, or a pharmaceutically acceptable salt thereof, wherein R^{8b} is

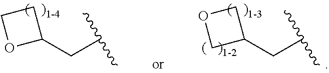

E28.21. The compound of E28.20, or a pharmaceutically acceptable salt thereof, wherein R is

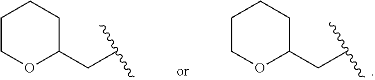 [SMILES: *CC1CCCCO1 ; *CC1CCCCO1]

E28.22. The compound of any of E1-E26, E28.5, E28.8, or E28.13-E28.14, or a pharmaceutically acceptable salt thereof, wherein R^{8b} is

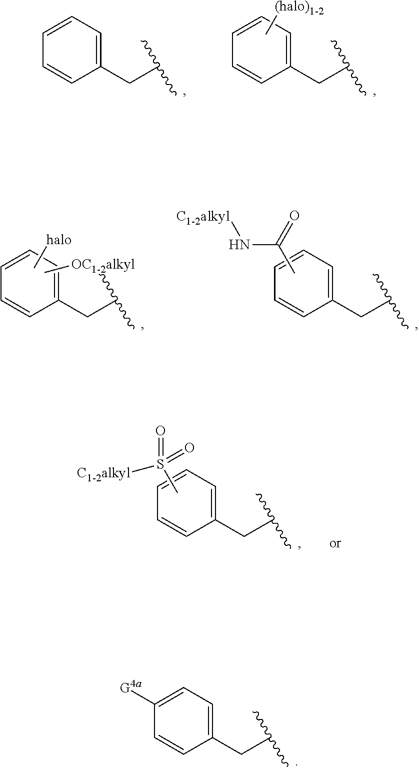

E28.23. The compound of E28.22, or a pharmaceutically acceptable salt thereof, wherein R^{8b} is

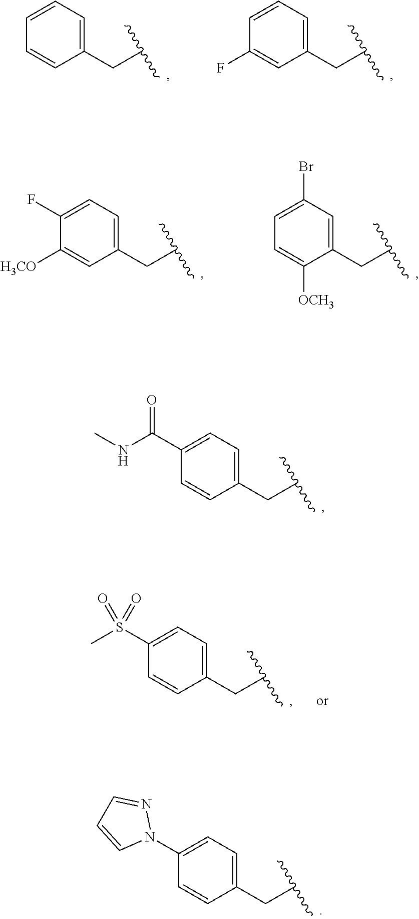

E28.24. The compound of any of E1-E26, E28.5, E28.9, or E28.13-E28.14, or a pharmaceutically acceptable salt thereof, wherein R^{8b} is

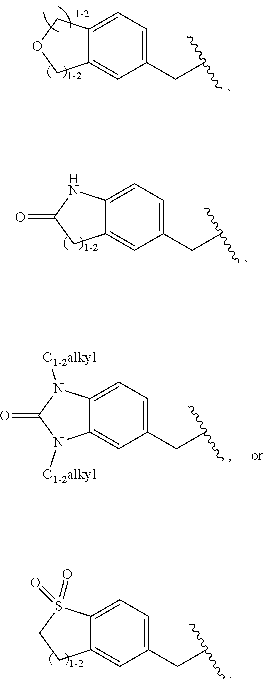

E28.25. The compound of E28.24, or a pharmaceutically acceptable salt thereof, wherein R^{gb} is

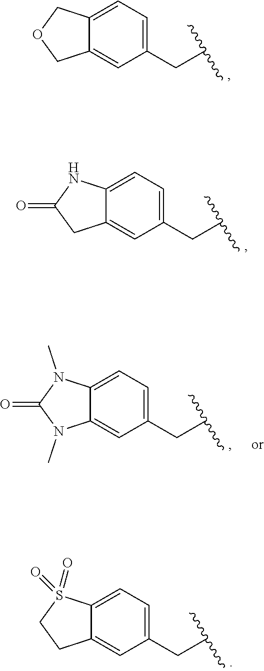 [SMILES: *Cc1ccc2c(c1)COC2 ; *Cc1ccc2c(c1)CC(=O)N2 ; *Cc1ccc2c(c1)n(C)c(=O)n2C ; *Cc1ccc2c(c1)CCS2(=O)=O]

E28.26. The compound of any of E1-E26, E28.5, E28.6, or E28.13-E28.14, or a pharmaceutically acceptable salt thereof, wherein R^{8b} is

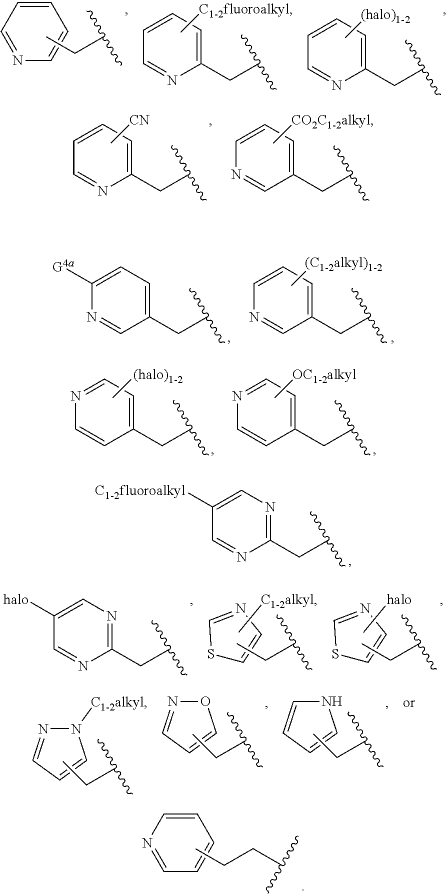

E28.27. The compound of E28.26, or a pharmaceutically acceptable salt thereof, wherein R^{8b} is

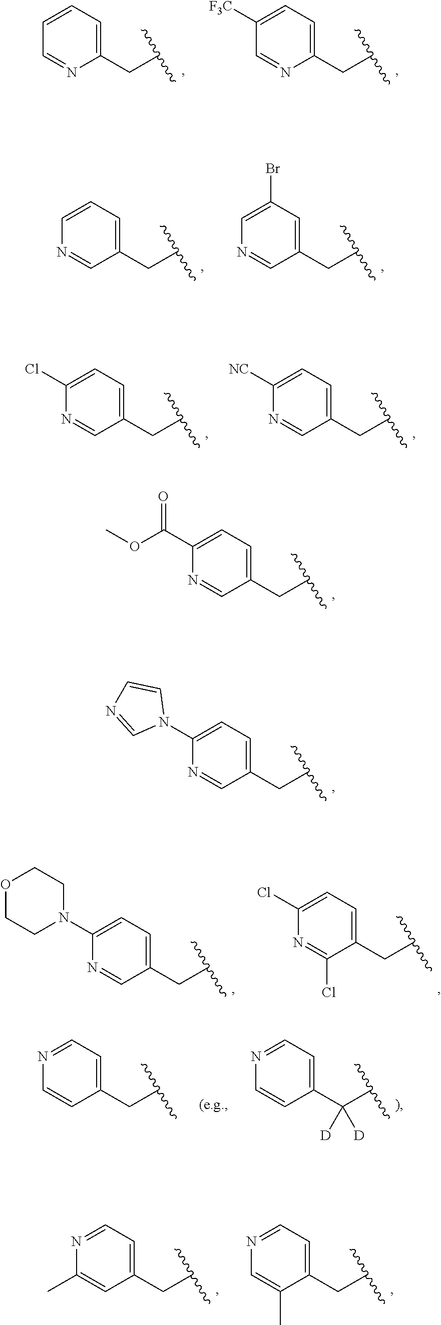

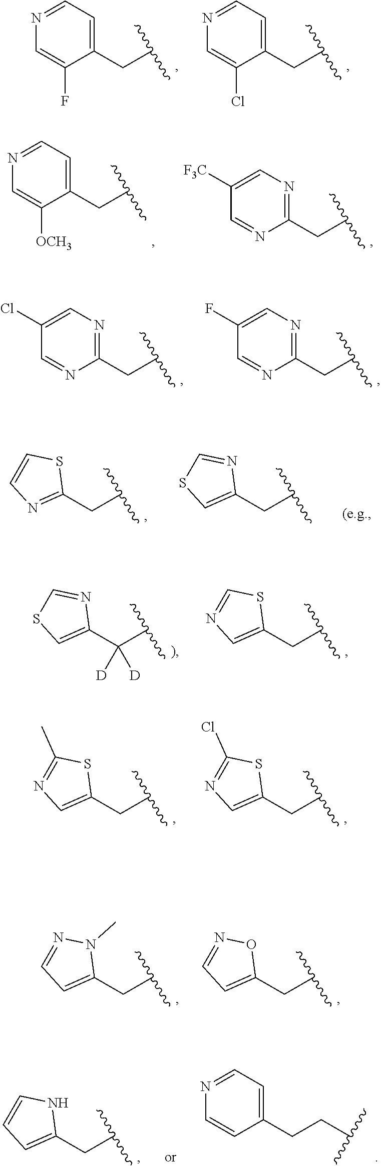

E28.28. The compound of any of E1-E26, E28.5, E28.7, or E28.13-E28.14, or a pharmaceutically acceptable salt thereof, wherein R^{8b} is

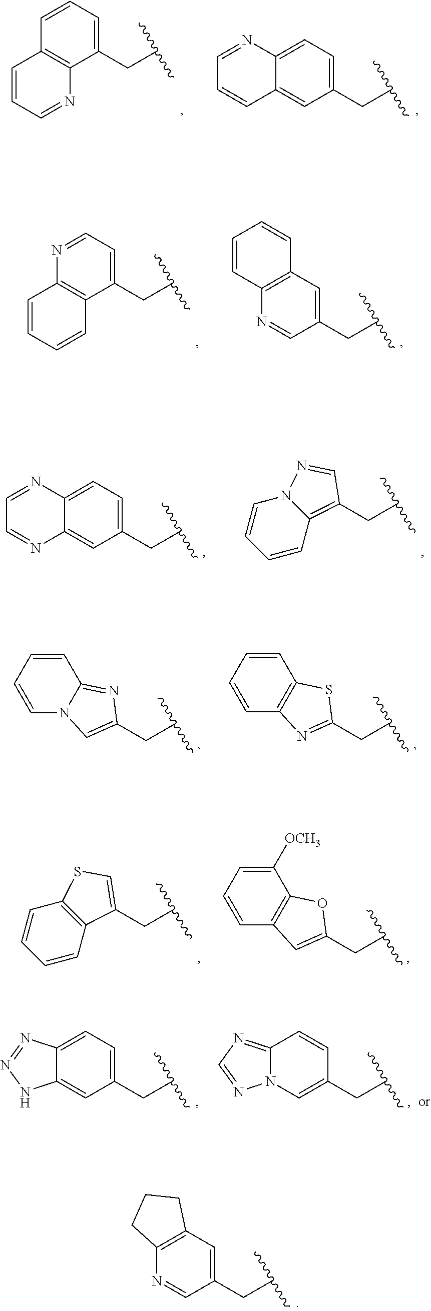

E28.29. The compound of any of E1-E26 or E28, or a pharmaceutically acceptable salt thereof, wherein R^{8b} is —C₁₋₆alkylene-OH (e.g., CH₂C(OH)(CH₃)₂).
E28.30. The compound of any of E1-E26 or E28-E28.29, or a pharmaceutically acceptable salt thereof, wherein G^{4a} is a phenyl, a 5- to 6-membered heteroaryl containing 1-3 heteroatoms, a 4- to 8-membered heterocyclyl containing 1-2 heteroatoms, or a 3- to 8-membered carbocyclyl, wherein the heteroatoms are independently O, N, or S, and G^{4a}, at each occurrence, is independently optionally substituted with 1-5 substituents independently selected from the group consisting of halogen, cyano, C₁₋₄alkyl, C₁₋₄haloalkyl, oxo, OH, —OC₁₋₄alkyl, and —OC₁₋₄haloalkyl.
E28.31. The compound of E28.30, or a pharmaceutically acceptable salt thereof, wherein G^{4a} is pyrazolyl, imidazolyl, morpholino, or pyridinyl.
E29. The compound of any of E1-E28.31, or a pharmaceutically acceptable salt thereof, wherein R^{8c} is hydrogen or C₁₋₄alkyl (e.g., methyl).
E29.1. The compound of E29, or a pharmaceutically acceptable salt thereof, wherein R^{8c} is hydrogen.
E30. The compound of any of E28-E28.31, or a pharmaceutically acceptable salt thereof, wherein R⁴ is —OR^{4a}, and R^{8c} and R^{4a}, together with the atom to which each attaches, form an oxazepin-5-one ring

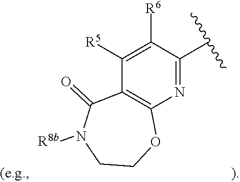

E31. The compound of E26, or a pharmaceutically acceptable salt thereof, wherein R⁸ is cyano.
E32. The compound of any of E1-E31, or a pharmaceutically acceptable salt thereof, wherein n is 0, i.e., formula (I) is:

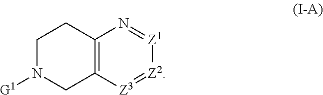

E32.1. The compound of E32, or a pharmaceutically acceptable salt thereof, wherein formula (I-A) is

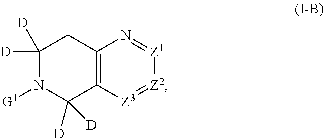

i.e., the hydrogen atoms adjacent the non-aromatic ring nitrogen atom are the isotope deuterium.
E33. The compound of any of E1-E31, or a pharmaceutically acceptable salt thereof, wherein n is 1.
E33.1. The compound of E33, or a pharmaceutically acceptable salt thereof, wherein the compound has formula (II):

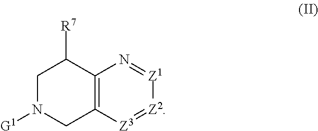

E33.2. The compound of E33.1, or a pharmaceutically acceptable salt thereof, wherein the compound has formula (II-A):

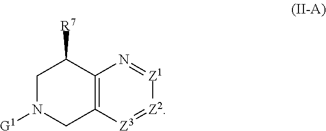

E33.3. The compound of E33.1, or a pharmaceutically acceptable salt thereof, wherein the compound has formula (II-B):

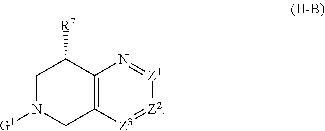

E33.4. The compound of E33, or a pharmaceutically acceptable salt thereof, wherein the compound has formula (III):

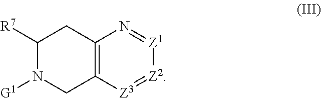

E34. The compound of any of E1-E31, or a pharmaceutically acceptable salt thereof, wherein n is 2.
E34.1. The compound of E34, or a pharmaceutically acceptable salt thereof, wherein the compound has formula (IV):

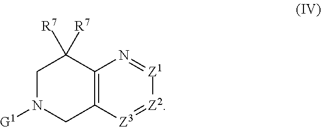

E35. The compound of any of E1-E34.1, or a pharmaceutically acceptable salt thereof, wherein R⁷ is C₁.4alkyl.
E35.1. The compound of E35, or a pharmaceutically acceptable salt thereof, wherein R⁷ is methyl.
E36. The compound of E1 selected from the group consisting of

5-methyl-6-(3-pyrrolidin-1-yl-7,8-dihydro-5H-1,6-naphthyridin-6-yl)pyridine-3-carbonitrile
6-[3-(4-fluoroanilino)-7,8-dihydro-5H-1,6-naphthyridin-6-yl]-5-methyl-pyridine-3-
carbonitrile
6-[3-(4-fluorophenyl)-7,8-dihydro-5H-1,6-naphthyridin-6-yl]-5-methyl-pyridine-3-
carbonitrile
6-[3-[(4-fluorophenyl)methylamino]-7,8-dihydro-5H-1,6-naphthyridin-6-yl]-5-methyl-
pyridine-3-carbonitrile
6-[3-[(3-fluoro-4-methoxy-phenyl)methylamino]-7,8-dihydro-5H-1,6-naphthyridin-6-yl]-5-
methyl-pyridine-3-carbonitrile
5-methyl-6-[3-(2-pyridylamino)-7,8-dihydro-5H-1,6-naphthyridin-6-yl]pyridine-3-
carbonitrile
6-[3-(2-fluoroanilino)-7,8-dihydro-5H-1,6-naphthyridin-6-yl]-5-methyl-pyridine-3-
carbonitrile
6-[3-(1,4-dioxan-2-ylmethylamino)-7,8-dihydro-5H-1,6-naphthyridin-6-yl]-5-methyl-
pyridine-3-carbonitrile
5-methyl-6-[3-(tetrahydrofuran-2-ylmethylamino)-7,8-dihydro-5H-1,6-naphthyridin-6-
yl]pyridine-3-carbonitrile
6-[3-[1-(cyclopropylmethyl)pyrazol-3-yl]-7,8-dihydro-5H-1,6-naphthyridin-6-yl]-4,5-
dimethyl-pyridazine-3-carbonitrile
4,5-dimethyl-6-[3-(1-methylpyrazol-4-yl)-7,8-dihydro-5H-1,6-naphthyridin-6-
yl]pyridazine-3-carbonitrile
4,5-dimethyl-6-[3-(2-methyl-4-pyridyl)-7,8-dihydro-5H-1,6-naphthyridin-6-yl]pyridazine-
3-carbonitrile
6-[3-(1-cyclopropylpyrazol-4-yl)-7,8-dihydro-5H-1,6-naphthyridin-6-yl]-4,5-dimethyl-
pyridazine-3-carbonitrile
4,5-dimethyl-6-[3-(2-methylpyrazol-3-yl)-7,8-dihydro-5H-1,6-naphthyridin-6-
yl]pyridazine-3-carbonitrile
6-[3-(1-cyclohexylpyrazol-3-yl)-7,8-dihydro-5H-1,6-naphthyridin-6-yl]-4,5-dimethyl-
pyridazine-3-carbonitrile
4,5-dimethyl-6-[3-(1-propylpyrazol-4-yl)-7,8-dihydro-5H-1,6-naphthyridin-6-
yl]pyridazine-3-carbonitrile
4,5-dimethyl-6-[3-(3-methylbenzimidazol-5-yl)-7,8-dihydro-5H-1,6-naphthyridin-6-
yl]pyridazine-3-carbonitrile
6-[3-(2-fluorophenyl)-7,8-dihydro-5H-1,6-naphthyridin-6-yl]-4,5-dimethyl-pyridazine-3-
carbonitrile
6-(3-imidazo[1,2-a]pyridin-6-yl-7,8-dihydro-5H-1,6-naphthyridin-6-yl)-4,5-dimethyl-
pyridazine-3-carbonitrile
4,5-dimethyl-6-[3-(2-phenylethyl)-7,8-dihydro-5H-1,6-naphthyridin-6-yl]pyridazine-3-
carbonitrile
6-[3-(3-fluorophenyl)-7,8-dihydro-5H-1,6-naphthyridin-6-yl]-4,5-dimethyl-pyridazine-3-
carbonitrile
4,5-dimethyl-6-(3-pyrrolidin-1-yl-7,8-dihydro-5H-1,6-naphthyridin-6-yl)pyridazine-3-
carbonitrile
4,5-dimethyl-6-[3-(1-piperidyl)-7,8-dihydro-5H-1,6-naphthyridin-6-yl]pyridazine-3-
carbonitrile
6-[3-[(3-fluorophenyl)methylamino]-7,8-dihydro-5H-1,6-naphthyridin-6-yl]-4,5-dimethyl-
pyridazine-3-carbonitrile
6-[3-[(4-methoxyphenyl)methylamino]-7,8-dihydro-5H-1,6-naphthyridin-6-yl]-4,5-
dimethyl-pyridazine-3-carbonitrile
6-[3-[(4-cyanophenyl)methylamino]-7,8-dihydro-5H-1,6-naphthyridin-6-yl]-4,5-dimethyl-
pyridazine-3-carbonitrile
6-[3-[(3-methoxyphenyl)methylamino]-7,8-dihydro-5H-1,6-naphthyridin-6-yl]-4,5-
dimethyl-pyridazine-3-carbonitrile
6-[3-(3,3-dimethylazetidin-1-yl)-7,8-dihydro-5H-1,6-naphthyridin-6-yl]-4,5-dimethyl-
pyridazine-3-carbonitrile
4,5-dimethyl-6-[3-[(6-methyl-3-pyridyl)methylamino]-7,8-dihydro-5H-1,6-naphthyridin-6-
yl]pyridazine-3-carbonitrile
6-[3-(2-fluoroanilino)-7,8-dihydro-5H-1,6-naphthyridin-6-yl]-4,5-dimethyl-pyridazine-3-
carbonitrile
4,5-dimethyl-6-(3-thiazol-5-yl-7,8-dihydro-5H-1,6-naphthyridin-6-yl)pyridazine-3-
carbonitrile
6-[3-(5-cyano-2-fluoro-anilino)-7,8-dihydro-5H-1,6-naphthyridin-6-yl]-4,5-dimethyl-
pyridazine-3-carbonitrile
6-[3-(2-chloro-6-fluoro-anilino)-7,8-dihydro-5H-1,6-naphthyridin-6-yl]-4,5-dimethyl-
pyridazine-3-carbonitrile
6-[3-(2-fluoro-3-methoxy-anilino)-7,8-dihydro-5H-1,6-naphthyridin-6-yl]-4,5-dimethyl-
pyridazine-3-carbonitrile
6-[3-[2-fluoro-3-(trifluoromethyl)anilino]-7,8-dihydro-5H-1,6-naphthyridin-6-yl]-4,5-
dimethyl-pyridazine-3-carbonitrile
6-[3-(3-chloro-2-fluoro-anilino)-7,8-dihydro-5H-1,6-naphthyridin-6-yl]-4,5-dimethyl-
pyridazine-3-carbonitrile
6-[3-(2-fluoro-5-methyl-anilino)-7,8-dihydro-5H-1,6-naphthyridin-6-yl]-4,5-dimethyl-
pyridazine-3-carbonitrile
6-[3-(2,4-difluoroanilino)-7,8-dihydro-5H-1,6-naphthyridin-6-yl]-4,5-dimethyl-pyridazine-
3-carbonitrile
6-[3-(2-chloroanilino)-7,8-dihydro-5H-1,6-naphthyridin-6-yl]-4,5-dimethyl-pyridazine-3-
carbonitrile
6-[3-[2-(dimethylamino)anilino]-7,8-dihydro-5H-1,6-naphthyridin-6-yl]-4,5-dimethyl-
pyridazine-3-carbonitrile
6-[3-(2-cyanoanilino)-7,8-dihydro-5H-1,6-naphthyridin-6-yl]-4,5-dimethyl-pyridazine-3-
carbonitrile
4,5-dimethyl-6-[3-(2-methylanilino)-7,8-dihydro-5H-1,6-naphthyridin-6-yl]pyridazine-3-
carbonitrile
4,5-dimethyl-6-[3-[2-(trifluoromethyl)anilino]-7,8-dihydro-5H-1,6-naphthyridin-6-
yl]pyridazine-3-carbonitrile
4,5-dimethyl-6-[3-[2-(trifluoromethoxy)anilino]-7,8-dihydro-5H-1,6-naphthyridin-6-
yl]pyridazine-3-carbonitrile
6-(7,8-dihydro-5H-1,6-naphthyridin-6-yl)-5-methyl-pyridine-3-carbonitrile
6-[3-(4-chloro-2-fluoro-anilino)-7,8-dihydro-5H-1,6-naphthyridin-6-yl]-4,5-dimethyl-
pyridazine-3-carbonitrile
4,5-dimethyl-6-[3-(3-pyridyl)-7,8-dihydro-5H-1,6-naphthyridin-6-yl]pyridazine-3-
carbonitrile
4,5-dimethyl-6-(3-pyridazin-4-yl-7,8-dihydro-5H-1,6-naphthyridin-6-yl)pyridazine-3-
carbonitrile
4,5-dimethyl-6-(3-pyrimidin-5-yl-7,8-dihydro-5H-1,6-naphthyridin-6-yl)pyridazine-3-
carbonitrile
4,5-dimethyl-6-[3-[2-(trifluoromethyl)-3-pyridyl]-7,8-dihydro-5H-1,6-naphthyridin-6-
yl]pyridazine-3-carbonitrile
6-[3-(6-chloro-5-fluoro-3-pyridyl)-7,8-dihydro-5H-1,6-naphthyridin-6-yl]-4,5-dimethyl-
pyridazine-3-carbonitrile
6-[3-(2-chloro-5-methoxy-3-pyridyl)-7,8-dihydro-5H-1,6-naphthyridin-6-yl]-4,5-dimethyl-
pyridazine-3-carbonitrile
6-[3-(2-chloro-5-fluoro-3-pyridyl)-7,8-dihydro-5H-1,6-naphthyridin-6-yl]-4,5-dimethyl-
pyridazine-3-carbonitrile
6-[3-(2-fluoro-3-pyridyl)-7,8-dihydro-5H-1,6-naphthyridin-6-yl]-4,5-dimethyl-pyridazine-
3-carbonitrile
4,5-dimethyl-6-[3-(4-methyl-3-pyridyl)-7,8-dihydro-5H-1,6-naphthyridin-6-yl]pyridazine-
3-carbonitrile
6-[3-(6-chloro-3-pyridyl)-7,8-dihydro-5H-1,6-naphthyridin-6-yl]-4,5-dimethyl-pyridazine-
3-carbonitrile
6-(7,8-dihydro-5H-1,6-naphthyridin-6-yl)-N,5-dimethyl-pyridine-3-carboxamide
6-(7,8-dihydro-5H-1,6-naphthyridin-6-yl)-N-isopropyl-5-methyl-pyridine-3-carboxamide
6-(7,8-dihydro-5H-1,6-naphthyridin-6-yl)-5-methyl-N-(2-pyridylmethyl)pyridine-3-
carboxamide
6-(7,8-dihydro-5H-1,6-naphthyridin-6-yl)-5-methyl-N-(3-pyridylmethyl)pyridine-3-
carboxamide
6-(7,8-dihydro-5H-1,6-naphthyridin-6-yl)-5-methyl-N-(4-pyridylmethyl)pyridine-3-
carboxamide
6-(7,8-dihydro-5H-1,6-naphthyridin-6-yl)-5-methyl-N-(thiazol-5-ylmethyl)pyridine-3-
carboxamide
6-(7,8-dihydro-5H-1,6-naphthyridin-6-yl)-5-methyl-N-[(2-methylpyrazol-3-
yl)methyl]pyridine-3-carboxamide
N-cyclopropyl-6-(7,8-dihydro-5H-1,6-naphthyridin-6-yl)-5-methyl-pyridine-3-carboxamide
N-cyclopentyl-6-(7,8-dihydro-5H-1,6-naphthyridin-6-yl)-5-methyl-pyridine-3-carboxamide
N-cyclohexyl-6-(7,8-dihydro-5H-1,6-naphthyridin-6-yl)-5-methyl-pyridine-3-carboxamide
6-(7,8-dihydro-5H-1,6-naphthyridin-6-yl)-5-methyl-N-[(4-pyrazol-1-
ylphenyl)methyl]pyridine-3-carboxamide
6-(7,8-dihydro-5H-1,6-naphthyridin-6-yl)-5-methyl-N-(6-quinolylmethyl)pyridine-3-
carboxamide
6-(7,8-dihydro-5H-1,6-naphthyridin-6-yl)-5-methyl-N-(pyrazolo[1,5-a]pyridin-3-
ylmethyl)pyridine-3-carboxamide
N-benzyl-6-(7,8-dihydro-5H-1,6-naphthyridin-6-yl)-5-methyl-pyridine-3-carboxamide
methyl 6-(7,8-dihydro-5H-1,6-naphthyridin-6-yl)-5-methyl-pyridine-3-carboxylate
6-[3-(2-fluoro-5-methyl-4-pyridyl)-7,8-dihydro-5H-1,6-naphthyridin-6-yl]-4,5-dimethyl-
pyridazine-3-carbonitrile
6-[3-(2-chloro-3-fluoro-4-pyridyl)-7,8-dihydro-5H-1,6-naphthyridin-6-yl]-4,5-dimethyl-
pyridazine-3-carbonitrile
4,5-dimethyl-6-[3-(4-pyridyl)-7,8-dihydro-5H-1,6-naphthyridin-6-yl]pyridazine-3-
carbonitrile
6-[3-(2,6-difluoro-4-pyridyl)-7,8-dihydro-5H-1,6-naphthyridin-6-yl]-4,5-dimethyl-
pyridazine-3-carbonitrile
6-[3-(2-methoxy-4-pyridyl)-7,8-dihydro-5H-1,6-naphthyridin-6-yl]-4,5-dimethyl-
pyridazine-3-carbonitrile
4,5-dimethyl-6-[3-(3-methyl-4-pyridyl)-7,8-dihydro-5H-1,6-naphthyridin-6-yl]pyridazine-
3-carbonitrile
6-[3-[(3,5-difluoro-2-pyridyl)amino]-7,8-dihydro-5H-1,6-naphthyridin-6-yl]-4,5-dimethyl-
pyridazine-3-carbonitrile
6-[3-[(2-fluoro-6-methyl-3-pyridyl)amino]-7,8-dihydro-5H-1,6-naphthyridin-6-yl]-4,5-
dimethyl-pyridazine-3-carbonitrile
6-[3-[(3-fluoro-2-pyridyl)amino]-7,8-dihydro-5H-1,6-naphthyridin-6-yl]-4,5-dimethyl-
pyridazine-3-carbonitrile
6-[3-[(2-chloro-5-fluoro-pyrimidin-4-yl)amino]-7,8-dihydro-5H-1,6-naphthyridin-6-yl]-4,5-
dimethyl-pyridazine-3-carbonitrile
6-[3-[(5-chloro-2-fluoro-3-pyridyl)amino]-7,8-dihydro-5H-1,6-naphthyridin-6-yl]-4,5-
dimethyl-pyridazine-3-carbonitrile
6-(6-chloro-2,5-dimethyl-pyrimidin-4-yl)-7,8-dihydro-5H-1,6-naphthyridine
6-(6-chloro-2-cyclopropyl-5-methyl-pyrimidin-4-yl)-7,8-dihydro-5H-1,6-naphthyridine
6-(6-chloro-2,5-dimethyl-pyrimidin-4-yl)-N-(2-fluorophenyl)-7,8-dihydro-5H-1,6-
naphthyridin-3-amine
6-(6-chloro-2,5-dimethyl-pyrimidin-4-yl)-N-(2,3-difluorophenyl)-7,8-dihydro-5H-1,6-
naphthyridin-3-amine
6-(6-chloro-2,5-dimethyl-pyrimidin-4-yl)-N-(2,4-difluorophenyl)-7,8-dihydro-5H-1,6-
naphthyridin-3-amine
6-(6-chloro-2,5-dimethyl-pyrimidin-4-yl)-N-(2,5-difluorophenyl)-7,8-dihydro-5H-1,6-
naphthyridin-3-amine
6-(6-chloro-2,5-dimethyl-pyrimidin-4-yl)-N-(2,6-difluorophenyl)-7,8-dihydro-5H-1,6-
naphthyridin-3-amine
6-(6-chloro-2-cyclopropyl-5-methyl-pyrimidin-4-yl)-N-(2-fluorophenyl)-7,8-dihydro-5H-
1,6-naphthyridin-3-amine
6-(6-chloro-2-cyclopropyl-5-methyl-pyrimidin-4-yl)-N-(2,3-difluorophenyl)-7,8-dihydro-
5H-1,6-naphthyridin-3-amine
6-(6-chloro-2,5-dimethyl-pyrimidin-4-yl)-3-(1-methylpyrazol-4-yl)-7,8-dihydro-5H-1,6-
naphthyridine
6-(6-chloro-2,5-dimethyl-pyrimidin-4-yl)-3-(2-methyl-4-pyridyl)-7,8-dihydro-5H-1,6-
naphthyridine
5-[6-(6-chloro-2,5-dimethyl-pyrimidin-4-yl)-7,8-dihydro-5H-1,6-naphthyridin-3-yl]thiazole
5-methyl-6-(3-thiazol-5-yl-7,8-dihydro-5H-1,6-naphthyridin-6-yl)pyridine-3-carbonitrile
6-[3-(1-cyclopropylpyrazol-4-yl)-7,8-dihydro-5H-1,6-naphthyridin-6-yl]-5-methyl-
pyridine-3-carbonitrile
6-[3-(1-benzylpyrazol-4-yl)-7,8-dihydro-5H-1,6-naphthyridin-6-yl]-5-methyl-pyridine-3-
carbonitrile
6-[3-(1-cyclopentylpyrazol-4-yl)-7,8-dihydro-5H-1,6-naphthyridin-6-yl]-5-methyl-pyridine-
3-carbonitrile
6-[3-(1-cyclohexylpyrazol-4-yl)-7,8-dihydro-5H-1,6-naphthyridin-6-yl]-5-methyl-pyridine-
3-carbonitrile
5-methyl-6-[3-[1-(2,2,2-trifluoroethyl)pyrazol-4-yl]-7,8-dihydro-5H-1,6-naphthyridin-6-
yl]pyridine-3-carbonitrile
6-[3-(1-cyclobutylpyrazol-4-yl)-7,8-dihydro-5H-1,6-naphthyridin-6-yl]-5-methyl-pyridine-
3-carbonitrile
6-[3-[1-(cyclopropylmethyl)pyrazol-4-yl]-7,8-dihydro-5H-1,6-naphthyridin-6-yl]-5-methyl-
pyridine-3-carbonitrile
6-[3-(1,3-dimethylpyrazol-4-yl)-7,8-dihydro-5H-1,6-naphthyridin-6-yl]-5-methyl-pyridine-
3-carbonitrile
5-methyl-6-[3-(1-methylpyrazol-4-yl)-7,8-dihydro-5H-1,6-naphthyridin-6-yl]pyridine-3-
carbonitrile
6-[3-(1-ethylpyrazol-4-yl)-7,8-dihydro-5H-1,6-naphthyridin-6-yl]-5-methyl-pyridine-3-
carbonitrile
6-[3-[1-(4-fluorophenyl)pyrazol-4-yl]-7,8-dihydro-5H-1,6-naphthyridin-6-yl]-5-methyl-
pyridine-3-carbonitrile
5-methyl-6-[3-(1,3,5-trimethylpyrazol-4-yl)-7,8-dihydro-5H-1,6-naphthyridin-6-
yl]pyridine-3-carbonitrile
5-methyl-6-[3-(1-propylpyrazol-4-yl)-7,8-dihydro-5H-1,6-naphthyridin-6-yl]pyridine-3-
carbonitrile
6-[3-(3,5-dimethylisoxazol-4-yl)-7,8-dihydro-5H-1,6-naphthyridin-6-yl]-5-methyl-pyridine-
3-carbonitrile
6-[3-(2,5-difluoroanilino)-7,8-dihydro-5H-1,6-naphthyridin-6-yl]-5-methyl-pyridine-3-
carbonitrile
6-[3-(2-chloro-6-fluoro-anilino)-7,8-dihydro-5H-1,6-naphthyridin-6-yl]-5-methyl-pyridine-
3-carbonitrile
6-[3-(1-isobutylpyrazol-4-yl)-7,8-dihydro-5H-1,6-naphthyridin-6-yl]-5-methyl-pyridine-3-
carbonitrile
6-[3-[2-fluoro-3-(trifluoromethyl)anilino]-7,8-dihydro-5H-1,6-naphthyridin-6-yl]-5-methyl-
pyridine-3-carbonitrile
6-[3-(3-chloro-2-fluoro-anilino)-7,8-dihydro-5H-1,6-naphthyridin-6-yl]-5-methyl-pyridine-
3-carbonitrile
6-[3-(2-fluoro-5-methyl-anilino)-7,8-dihydro-5H-1,6-naphthyridin-6-yl]-5-methyl-pyridine-
3-carbonitrile
5-methyl-6-[3-(2,3,5-trifluoroanilino)-7,8-dihydro-5H-1,6-naphthyridin-6-yl]pyridine-3-
carbonitrile
4,5-dimethyl-6-(3-methyl-7,8-dihydro-5H-1,6-naphthyridin-6-yl)pyridazine-3-carbonitrile
6-(7,8-dihydro-5H-1,6-naphthyridin-6-yl)-4,5-dimethyl-pyridazine-3-carbonitrile
6-[3-[(2-fluoro-3-pyridyl)amino]-7,8-dihydro-5H-1,6-naphthyridin-6-yl]-4,5-dimethyl-
pyridazine-3-carbonitrile
6-(7,8-dihydro-5H-1,6-naphthyridin-6-yl)-N-(imidazo[1,2-a]pyridin-2-ylmethyl)-5-methyl-
pyridine-3-carboxamide
6-(7,8-dihydro-5H-1,6-naphthyridin-6-yl)-5-methyl-N-(4-pyridyl)pyridine-3-carboxamide
6-(7,8-dihydro-5H-1,6-naphthyridin-6-yl)-5-methyl-N-tetrahydropyran-4-yl-pyridine-3-
carboxamide
6-(7,8-dihydro-5H-1,6-naphthyridin-6-yl)-N-(isoxazol-5-ylmethyl)-5-methyl-pyridine-3-
carboxamide
6-(7,8-dihydro-5H-1,6-naphthyridin-6-yl)-N-(3-fluoro-1-bicyclo[1.1.1]pentanyl)-5-methyl-
pyridine-3-carboxamide
N-(1,3-benzothiazol-2-ylmethyl)-6-(7,8-dihydro-5H-1,6-naphthyridin-6-yl)-5-methyl-
pyridine-3-carboxamide
6-(7,8-dihydro-5H-1,6-naphthyridin-6-yl)-5-methyl-N-[2-(4-pyridyl)ethyl]pyridine-3-
carboxamide
6-(7,8-dihydro-5H-1,6-naphthyridin-6-yl)-5-methyl-N-[(2-methylthiazol-5-
yl)methyl]pyridine-3-carboxamide
N-(benzothiophen-3-ylmethyl)-6-(7,8-dihydro-5H-1,6-naphthyridin-6-yl)-5-methyl-
pyridine-3-carboxamide
6-(7,8-dihydro-5H-1,6-naphthyridin-6-yl)-5-methyl-N-[(2-methyl-4-
pyridyl)methyl]pyridine-3-carboxamide
6-(7,8-dihydro-5H-1,6-naphthyridin-6-yl)-5-methyl-N-[(3-methyl-4-
pyridyl)methyl]pyridine-3-carboxamide
6-(7,8-dihydro-5H-1,6-naphthyridin-6-yl)-5-methyl-N-(4-quinolylmethyl)pyridine-3-
carboxamide
6-[3-[(2-fluoro-6-methyl-3-pyridyl)amino]-7,8-dihydro-5H-1,6-naphthyridin-6-yl]-5-
methyl-pyridine-3-carbonitrile
methyl 6-(3-bromo-7,8-dihydro-5H-1,6-naphthyridin-6-yl)-5-methyl-pyridine-3-
carboxylate
N-[(2-chlorothiazol-5-yl)methyl]-6-(7,8-dihydro-5H-1,6-naphthyridin-6-yl)-5-methyl-
pyridine-3-carboxamide
6-(7,8-dihydro-5H-1,6-naphthyridin-6-yl)-4,5-dimethyl-N-(4-pyridylmethyl)pyridazine-3-
carboxamide
6-(7,8-dihydro-5H-1,6-naphthyridin-6-yl)-4,5-dimethyl-N-(3-pyridylmethyl)pyridazine-3-
carboxamide
6-(7,8-dihydro-5H-1,6-naphthyridin-6-yl)-4,5-dimethyl-N-(2-pyridylmethyl)pyridazine-3-
carboxamide
6-(7,8-dihydro-5H-1,6-naphthyridin-6-yl)-4,5-dimethyl-N-(thiazol-5-ylmethyl)pyridazine-
3-carboxamide
N-[(5-bromo-3-pyridyl)methyl]-6-(7,8-dihydro-5H-1,6-naphthyridin-6-yl)-5-methyl-
pyridine-3-carboxamide
6-(7,8-dihydro-5H-1,6-naphthyridin-6-yl)-5-methyl-N-(3-quinolylmethyl)pyridine-3-
carboxamide
N-[(5-bromo-2-methoxy-phenyl)methyl]-6-(7,8-dihydro-5H-1,6-naphthyridin-6-yl)-5-
methyl-pyridine-3-carboxamide
methyl 5-[[[6-(7,8-dihydro-5H-1,6-naphthyridin-6-yl)-5-methyl-pyridine-3-
carbonyl]amino]methyl]pyridine-2-carboxylate
6-(7,8-dihydro-5H-1,6-naphthyridin-6-yl)-5-methyl-N-[(4-
methylsulfonylphenyl)methyl]pyridine-3-carboxamide
6-(7,8-dihydro-5H-1,6-naphthyridin-6-yl)-5-methyl-N-[[5-(trifluoromethyl)-2-
pyridyl]methyl]pyridine-3-carboxamide
6-(7,8-dihydro-5H-1,6-naphthyridin-6-yl)-5-methyl-N-[[5-(trifluoromethyl)pyrimidin-2-
yl]methyl]pyridine-3-carboxamide
N-[(5-chloropyrimidin-2-yl)methyl]-6-(7,8-dihydro-5H-1,6-naphthyridin-6-yl)-5-methyl-
pyridine-3-carboxamide
6-(7,8-dihydro-5H-1,6-naphthyridin-6-yl)-N-[(5-fluoropyrimidin-2-yl)methyl]-5-methyl-
pyridine-3-carboxamide
6-(7,8-dihydro-5H-1,6-naphthyridin-6-yl)-N-[(6-imidazol-1-yl-3-pyridyl)methyl]-5-methyl-
pyridine-3-carboxamide
6-(7,8-dihydro-5H-1,6-naphthyridin-6-yl)-5-methyl-N-[[4-
(methylcarbamoyl)phenyl]methyl]pyridine-3-carboxamide
N-(3H-benzotriazol-5-ylmethyl)-6-(7,8-dihydro-5H-1,6-naphthyridin-6-yl)-5-methyl-
pyridine-3-carboxamide
6-(7,8-dihydro-5H-1,6-naphthyridin-6-yl)-5-methyl-N-([1,2,4]triazolo[1,5-a]pyridin-6-
ylmethyl)pyridine-3-carboxamide
6-(7,8-dihydro-5H-1,6-naphthyridin-6-yl)-N-[(4-fluoro-3-methoxy-phenyl)methyl]-5-
methyl-pyridine-3-carboxamide
N-(1,3-dihydroisobenzofuran-5-ylmethyl)-6-(7,8-dihydro-5H-1,6-naphthyridin-6-yl)-5-
methyl-pyridine-3-carboxamide
6-(7,8-dihydro-5H-1,6-naphthyridin-6-yl)-5-methyl-N-[(2-oxoindolin-5-yl)methyl]pyridine-
3-carboxamide
N-(6,7-dihydro-5H-cyclopenta[b]pyridin-3-ylmethyl)-6-(7,8-dihydro-5H-1,6-naphthyridin-
6-yl)-5-methyl-pyridine-3-carboxamide
6-(7,8-dihydro-5H-1,6-naphthyridin-6-yl)-5-methyl-N-(8-quinolylmethyl)pyridine-3-
carboxamide
6-(7,8-dihydro-5H-1,6-naphthyridin-6-yl)-5-methyl-N-(quinoxalin-6-ylmethyl)pyridine-3-
carboxamide
6-(7,8-dihydro-5H-1,6-naphthyridin-6-yl)-N-[(1,3-dimethyl-2-oxo-benzimidazol-5-
yl)methyl]-5-methyl-pyridine-3-carboxamide
6-(7,8-dihydro-5H-1,6-naphthyridin-6-yl)-N-[(3-fluorophenyl)methyl]-5-methyl-pyridine-3-
carboxamide
6-(7,8-dihydro-5H-1,6-naphthyridin-6-yl)-5-methyl-N-(tetrahydropyran-4-
ylmethyl)pyridine-3-carboxamide
6-(7,8-dihydro-5H-1,6-naphthyridin-6-yl)-5-methyl-N-(tetrahydropyran-2-
ylmethyl)pyridine-3-carboxamide
6-(7,8-dihydro-5H-1,6-naphthyridin-6-yl)-N-[(7-methoxybenzofuran-2-yl)methyl]-5-
methyl-pyridine-3-carboxamide
6-(7,8-dihydro-5H-1,6-naphthyridin-6-yl)-5-methyl-N-(1H-pyrrol-2-ylmethyl)pyridine-3-
carboxamide
6-(7,8-dihydro-5H-1,6-naphthyridin-6-yl)-N-[(1,1-dioxo-2,3-dihydrobenzothiophen-5-
yl)methyl]-5-methyl-pyridine-3-carboxamide
5-methyl-6-[3-(trifluoromethyl)-7,8-dihydro-5H-1,6-naphthyridin-6-yl]pyridine-3-
carbonitrile
6-[3-[(2-fluoro-3-pyridyl)amino]-7,8-dihydro-5H-1,6-naphthyridin-6-yl]-5-methyl-
pyridine-3-carbonitrile
6-[3-[(3-fluoro-2-pyridyl)amino]-7,8-dihydro-5H-1,6-naphthyridin-6-yl]-5-methyl-
pyridine-3-carbonitrile
6-[3-(6-fluoro-2-pyridyl)-7,8-dihydro-5H-1,6-naphthyridin-6-yl]-5-methyl-pyridine-3-
carbonitrile
4,5-dimethyl-6-[3-[(2-methylpyrazol-3-yl)amino]-7,8-dihydro-5H-1,6-naphthyridin-6-
yl]pyridazine-3-carbonitrile
6-[3-(1,3-dimethylpyrazol-4-yl)-7,8-dihydro-5H-1,6-naphthyridin-6-yl]-5-methyl-N-
(thiazol-2-ylmethyl)pyridine-3-carboxamide
5-methyl-6-[3-(1-methylpyrazol-3-yl)-7,8-dihydro-5H-1,6-naphthyridin-6-yl]-N-(thiazol-2-
ylmethyl)pyridine-3-carboxamide
6-[3-(3,5-dimethylisoxazol-4-yl)-7,8-dihydro-5H-1,6-naphthyridin-6-yl]-5-methyl-N-
(thiazol-2-ylmethyl)pyridine-3-carboxamide
5-methyl-6-[3-(1-methylpyrazol-4-yl)-7,8-dihydro-5H-1,6-naphthyridin-6-yl]-N-(thiazol-2-
ylmethyl)pyridine-3-carboxamide
6-[3-(6-fluoro-4-methyl-3-pyridyl)-7,8-dihydro-5H-1,6-naphthyridin-6-yl]-5-methyl-N-
(thiazol-2-ylmethyl)pyridine-3-carboxamide
6-[3-(2-fluoro-3-pyridyl)-7,8-dihydro-5H-1,6-naphthyridin-6-yl]-5-methyl-N-(thiazol-2-
ylmethyl)pyridine-3-carboxamide
5-methyl-6-[3-(1-methylpyrazol-4-yl)-7,8-dihydro-5H-1,6-naphthyridin-6-yl]-N-(4-
pyridylmethyl)pyridine-3-carboxamide
6-[3-(6-fluoro-4-methyl-3-pyridyl)-7,8-dihydro-5H-1,6-naphthyridin-6-yl]-5-methyl-N-(4-
pyridylmethyl)pyridine-3-carboxamide
6-[3-(2-fluoro-3-pyridyl)-7,8-dihydro-5H-1,6-naphthyridin-6-yl]-5-methyl-N-(4-
pyridylmethyl)pyridine-3-carboxamide
5-methyl-6-(3-methyl-7,8-dihydro-5H-1,6-naphthyridin-6-yl)pyridine-3-carbonitrile
6-[3-(2-fluorophenoxy)-7,8-dihydro-5H-1,6-naphthyridin-6-yl]-5-methyl-pyridine-3-
carbonitrile
6-[3-(3-fluorophenoxy)-7,8-dihydro-5H-1,6-naphthyridin-6-yl]-5-methyl-pyridine-3-
carbonitrile
6-[3-[(6-methoxy-3-pyridyl)oxy]-7,8-dihydro-5H-1,6-naphthyridin-6-yl]-5-methyl-
pyridine-3-carbonitrile
5-methyl-6-[3-(1-methylindazol-5-yl)oxy-7,8-dihydro-5H-1,6-naphthyridin-6-yl]pyridine-
3-carbonitrile
6-[3-(2,4-dimethylthiazol-5-yl)-7,8-dihydro-5H-1,6-naphthyridin-6-yl]-5-methyl-pyridine-
3-carbonitrile
5-methyl-6-[3-(2-methylthiazol-5-yl)-7,8-dihydro-5H-1,6-naphthyridin-6-yl]pyridine-3-
carbonitrile
6-[3-(6-cyclopropyl-3-pyridyl)-7,8-dihydro-5H-1,6-naphthyridin-6-yl]-5-methyl-pyridine-
3-carbonitrile
5-methyl-6-[3-(6-methyl-3-pyridyl)-7,8-dihydro-5H-1,6-naphthyridin-6-yl]pyridine-3-
carbonitrile
6-[3-(6-methoxy-3-pyridyl)-7,8-dihydro-5H-1,6-naphthyridin-6-yl]-5-methyl-pyridine-3-
carbonitrile
4,5-dimethyl-6-[3-(2-methyl-4-pyridyl)-7,8-dihydro-5H-1,6-naphthyridin-6-yl]-N-(4-
pyridylmethyl)pyridazine-3-carboxamide
6-[3-(2-fluoroanilino)-7,8-dihydro-5H-1,6-naphthyridin-6-yl]-4,5-dimethyl-N-(4-
pyridylmethyl)pyridazine-3-carboxamide
5-methyl-6-[3-(3-pyridyl)-7,8-dihydro-5H-1,6-naphthyridin-6-yl]pyridine-3-carbonitrile
6-(6-chloro-2,5-dimethyl-pyrimidin-4-yl)-3-(1,3-dimethylpyrazol-4-yl)-7,8-dihydro-5H-
1,6-naphthyridine
6-(6-chloro-2,5-dimethyl-pyrimidin-4-yl)-3-(1,3,5-trimethylpyrazol-4-yl)-7,8-dihydro-5H-
1,6-naphthyridine
6-(6-chloro-2,5-dimethyl-pyrimidin-4-yl)-3-[1-(cyclopropylmethyl)pyrazol-4-yl]-7,8-
dihydro-5H-1,6-naphthyridine
6-(6-chloro-2,5-dimethyl-pyrimidin-4-yl)-3-(1-cyclobutylpyrazol-4-yl)-7,8-dihydro-5H-1,6-
naphthyridine
3-(1,3-dimethylpyrazol-4-yl)-6-(6-methoxy-2,5-dimethyl-pyrimidin-4-yl)-7,8-dihydro-5H-
1,6-naphthyridine
4-[6-(6-chloro-2,5-dimethyl-pyrimidin-4-yl)-7,8-dihydro-5H-1,6-naphthyridin-3-yl]-3,5-
dimethyl-isoxazole
5-[6-(6-chloro-2,5-dimethyl-pyrimidin-4-yl)-7,8-dihydro-5H-1,6-naphthyridin-3-yl]-2,4-
dimethyl-thiazole
5-[6-(6-chloro-2,5-dimethyl-pyrimidin-4-yl)-7,8-dihydro-5H-1,6-naphthyridin-3-yl]-2-
methyl-thiazole
6-(6-chloro-2,5-dimethyl-pyrimidin-4-yl)-3-(trifluoromethyl)-7,8-dihydro-5H-1,6-
naphthyridine
3-(1,3-dimethylpyrazol-4-yl)-6-(2,5,6-trimethylpyrimidin-4-yl)-7,8-dihydro-5H-1,6-
naphthyridine
6-(6-chloro-2,5-dimethyl-pyrimidin-4-yl)-3-(4-fluorophenoxy)-7,8-dihydro-5H-1,6-
naphthyridine
6-(6-chloro-2,5-dimethyl-pyrimidin-4-yl)-3-(3-fluorophenoxy)-7,8-dihydro-5H-1,6-
naphthyridine
6-(6-chloro-2,5-dimethyl-pyrimidin-4-yl)-3-(2-fluorophenoxy)-7,8-dihydro-5H-1,6-
naphthyridine
6-(6-chloro-2,5-dimethyl-pyrimidin-4-yl)-3-[(6-methoxy-3-pyridyl)oxy]-7,8-dihydro-5H-
1,6-naphthyridine
6-(6-chloro-2,5-dimethyl-pyrimidin-4-yl)-3-(2,3-dihydro-1,4-benzodioxin-6-yloxy)-7,8-
dihydro-5H-1,6-naphthyridine
6-(6-chloro-2,5-dimethyl-pyrimidin-4-yl)-3-(3-pyridyloxy)-7,8-dihydro-5H-1,6-
naphthyridine
6-(6-chloro-2,5-dimethyl-pyrimidin-4-yl)-3-(1-methylindazol-5-yl)oxy-7,8-dihydro-5H-1,6-
naphthyridine
6-(6-chloro-2,5-dimethyl-pyrimidin-4-yl)-3-methyl-7,8-dihydro-5H-1,6-naphthyridine
6-(6-chloro-2,5-dimethylpyrimidin-4-yl)-3-(trifluoromethyl)-5,6,7,8-tetrahydro-1,6-
naphthyridine
3-bromo-6-(6-chloro-2-(methoxymethyl)-5-methylpyrimidin-4-yl)-5,6,7,8-tetrahydro-1,6-
naphthyridine
6-(6-chloro-2,5-dimethylpyrimidin-4-yl)-N-(1-methyl-1H-pyrazol-5-yl)-5,6,7,8-tetrahydro-
1,6-naphthyridin-3-amine
6-(6-chloro-2,5-dimethylpyrimidin-4-yl)-5,6,7,8-tetrahydro-1,6-naphthyridin-3-amine
6-(6-chloro-2-(methoxymethyl)-5-methylpyrimidin-4-yl)-3-(trifluoromethyl)-5,6,7,8-
tetrahydro-1,6-naphthyridine
6-(6-chloro-2,5-dimethylpyrimidin-4-yl)-3-(difluoromethyl)-5,6,7,8-tetrahydro-1,6-
naphthyridine
6-(6-chloro-2-(methoxymethyl)-5-methylpyrimidin-4-yl)-3-(1,3-dimethyl-1H-pyrazol-4-
yl)-5,6,7,8-tetrahydro-1,6-naphthyridine
5-(6-(6-chloro-2-(methoxymethyl)-5-methylpyrimidin-4-yl)-5,6,7,8-tetrahydro-1,6-
naphthyridin-3-yl)thiazole
6-(6-chloro-2,5-dimethylpyrimidin-4-yl)-2-(trifluoromethyl)-5,6,7,8-tetrahydro-1,6-
naphthyridine
6-(6-chloro-2,5-dimethylpyrimidin-4-yl)-8,8-dimethyl-5,6,7,8-tetrahydro-1,6-naphthyridin-
3-amine
5-(6-(6-chloro-2,5-dimethylpyrimidin-4-yl)-5,6,7,8-tetrahydro-1,6-naphthyridin-3-yl)-N,N-
dimethylpyridin-2-amine
(2,5-dimethyl-6-(3-(trifluoromethyl)-7,8-dihydro-1,6-naphthyridin-6(5H)-yl)pyrimidin-4-
yl)(morpholino)methanone
6-(6-chloro-2,5-dimethylpyrimidin-4-yl)-8-methyl-3-nitro-5,6,7,8-tetrahydro-1,6-
naphthyridine
6-(2,5-dimethyl-6-(pyrrolidin-1-yl)pyrimidin-4-yl)-3-(6-fluoropyridin-3-yl)-5,6,7,8-
tetrahydro-1,6-naphthyridine
6-(6-chloro-2,5-dimethylpyrimidin-4-yl)-3-(6-fluoropyridin-3-yl)-5,6,7,8-tetrahydro-1,6-
naphthyridine
6-(6-chloro-2,5-dimethylpyrimidin-4-yl)-3-cyclopropyl-5,6,7,8-tetrahydro-1,6-
naphthyridine
(4-(3-(1,3-dimethyl-1H-pyrazol-4-yl)-7,8-dihydro-1,6-naphthyridin-6(5H)-yl)-5,6-
dimethylpyrimidin-2-yl)methanol
4-(5-(6-(2,5,6-trimethylpyrimidin-4-yl)-5,6,7,8-tetrahydro-1,6-naphthyridin-3-yl)pyridin-2-
yl)morpholine
3-(6-(3,3-difluoropyrrolidin-1-yl)pyridin-3-yl)-6-(2,5,6-trimethylpyrimidin-4-yl)-5,6,7,8-
tetrahydro-1,6-naphthyridine
3-(6-fluoropyridin-3-yl)-6-(2,5,6-trimethylpyrimidin-4-yl)-5,6,7,8-tetrahydro-1,6-
naphthyridine
3-(6-methoxypyridin-3-yl)-6-(2,5,6-trimethylpyrimidin-4-yl)-5,6,7,8-tetrahydro-1,6-
naphthyridine
3-(6-(pyrrolidin-1-yl)pyridin-3-yl)-6-(2,5,6-trimethylpyrimidin-4-yl)-5,6,7,8-tetrahydro-1,6-
naphthyridine
3-(6-(1H-imidazol-1-yl)pyridin-3-yl)-6-(2,5,6-trimethylpyrimidin-4-yl)-5,6,7,8-tetrahydro-
1,6-naphthyridine
3-(6-(4-methylpiperazin-1-yl)pyridin-3-yl)-6-(2,5,6-trimethylpyrimidin-4-yl)-5,6,7,8-
tetrahydro-1,6-naphthyridine
3-(6-(azetidin-1-yl)pyridin-3-yl)-6-(2,5,6-trimethylpyrimidin-4-yl)-5,6,7,8-tetrahydro-1,6-
naphthyridine
3-(trifluoromethyl)-6-(2,5,6-trimethylpyrimidin-4-yl)-5,6,7,8-tetrahydro-1,6-naphthyridine
3-bromo-6-(6-chloro-2,5-dimethylpyrimidin-4-yl)-8,8-dimethyl-5,6,7,8-tetrahydro-1,6-
naphthyridine
6-(6-chloro-5-methyl-2-(trifluoromethyl)pyrimidin-4-yl)-3-(trifluoromethyl)-5,6,7,8-
tetrahydro-1,6-naphthyridine
6-(6-chloro-2,5-dimethylpyrimidin-4-yl)-3-(1,3-dimethyl-1H-pyrazol-4-yl)-8,8-dimethyl-
5,6,7,8-tetrahydro-1,6-naphthyridine
6-(6-chloro-2,5-dimethylpyrimidin-4-yl)-8,8-dimethyl-N-(1-methyl-1H-pyrazol-5-yl)-
5,6,7,8-tetrahydro-1,6-naphthyridin-3-amine
N-(2-fluoropyridin-3-yl)-6-(2,5,6-trimethylpyrimidin-4-yl)-5,6,7,8-tetrahydro-1,6-
naphthyridin-3-amine
N-(1-methyl-1H-pyrazol-5-yl)-6-(2,5,6-trimethylpyrimidin-4-yl)-5,6,7,8-tetrahydro-1,6-
naphthyridin-3-amine
6-(6-chloro-5-methyl-2-(trifluoromethyl)pyrimidin-4-yl)-3-cyclopropyl-5,6,7,8-tetrahydro-
1,6-naphthyridine
6-(6-chloro-2,5-dimethylpyrimidin-4-yl)-3-(6,7-dihydropyrazolo[1,5-a]pyrimidin-4(5H)-
yl)-5,6,7,8-tetrahydro-1,6-naphthyridine
3-(6,7-dihydropyrazolo[1,5-a]pyrimidin-4(5H)-yl)-6-(2,5,6-trimethylpyrimidin-4-yl)-
5,6,7,8-tetrahydro-1,6-naphthyridine
6-(6-chloro-5-methyl-2-(trifluoromethyl)pyrimidin-4-yl)-3-(1,3-dimethyl-1H-pyrazol-4-yl)-
5,6,7,8-tetrahydro-1,6-naphthyridine
5-(6-(6-chloro-5-methyl-2-(trifluoromethyl)pyrimidin-4-yl)-5,6,7,8-tetrahydro-1,6-
naphthyridin-3-yl)-N,N-dimethylpyridin-2-amine
6-(6-chloro-5-methyl-2-(trifluoromethyl)pyrimidin-4-yl)-N-(1-methyl-1H-pyrazol-5-yl)-
5,6,7,8-tetrahydro-1,6-naphthyridin-3-amine
6-(6-chloro-5-methyl-2-(trifluoromethyl)pyrimidin-4-yl)-3-(6,7-dihydropyrazolo[1,5-
alpyrimidin-4(5H)-yl)-5,6,7,8-tetrahydro-1,6-naphthyridine
6-(6-chloro-5-methyl-2-(trifluoromethyl)pyrimidin-4-yl)-N-(2-fluoropyridin-3-yl)-5,6,7,8-
tetrahydro-1,6-naphthyridin-3-amine
6-(6-chloro-2,5-dimethylpyrimidin-4-yl)-3-(6,7-dihydropyrazolo[1,5-a]pyrimidin-4(5H)-
yl)-8,8-dimethyl-5,6,7,8-tetrahydro-1,6-naphthyridine
6-(6-chloro-2,5-dimethylpyrimidin-4-yl)-N-(2-fluoropyridin-3-yl)-5,6,7,8-tetrahydro-1,6-
naphthyridin-3-amine
6-(5,6-dimethyl-2-(trifluoromethyl)pyrimidin-4-yl)-3-(trifluoromethyl)-5,6,7,8-tetrahydro-
1,6-naphthyridine
6-(2-chloro-5,6-dimethylpyrimidin-4-yl)-3-(trifluoromethyl)-5,6,7,8-tetrahydro-1,6-
naphthyridine
6-(2-chloro-5,6-dimethylpyrimidin-4-yl)-3-cyclopropyl-5,6,7,8-tetrahydro-1,6-
naphthyridine
6-(6-chloro-2,5-dimethylpyrimidin-4-yl)-8-methyl-5,6,7,8-tetrahydro-1,6-naphthyridin-3-
amine
6-(6-chloro-2,5-dimethylpyrimidin-4-yl)-8-methyl-3-(trifluoromethyl)-5,6,7,8-tetrahydro-
1,6-naphthyridine
6-(6-chloro-2,5-dimethylpyrimidin-4-yl)-8,8-dimethyl-3-(trifluoromethyl)-5,6,7,8-
tetrahydro-1,6-naphthyridine
3-(6,7-dihydropyrazolo[1,5-a]pyrimidin-4(5H)-yl)-6-(5,6-dimethyl-2-
(trifluoromethyl)pyrimidin-4-yl)-5,6,7,8-tetrahydro-1,6-naphthyridine
6-(2-chloro-6,7-dihydro-5H-cyclopenta[d]pyrimidin-4-yl)-3-(trifluoromethyl)-5,6,7,8-
tetrahydro-1,6-naphthyridine
3-(2-fluorophenoxy)-6-(2,5,6-trimethylpyrimidin-4-yl)-5,6,7,8-tetrahydro-1,6-naphthyridine
3-(2,3-difluorophenoxy)-6-(2,5,6-trimethylpyrimidin-4-yl)-5,6,7,8-tetrahydro-1,6-
naphthyridine
3-(2,4-difluorophenoxy)-6-(2,5,6-trimethylpyrimidin-4-yl)-5,6,7,8-tetrahydro-1,6-
naphthyridine
3-(2,5-difluorophenoxy)-6-(2,5,6-trimethylpyrimidin-4-yl)-5,6,7,8-tetrahydro-1,6-
naphthyridine
2-chloro-4-(3-(trifluoromethyl)-7,8-dihydro-1,6-naphthyridin-6(5H)-yl)-5,6,7,8-
tetrahydroquinazoline
6-(2-cyclopropyl-6,7-dihydro-5H-cyclopenta[d]pyrimidin-4-yl)-3-(trifluoromethyl)-5,6,7,8-
tetrahydro-1,6-naphthyridine
2-cyclopropyl-4-(3-(trifluoromethyl)-7,8-dihydro-1,6-naphthyridin-6(5H)-yl)-5,6,7,8-
tetrahydroquinazoline
6-(2-methyl-6,7-dihydro-5H-cyclopenta[d]pyrimidin-4-yl)-3-(trifluoromethyl)-5,6,7,8-
tetrahydro-1,6-naphthyridine
2-methyl-4-(3-(trifluoromethyl)-7,8-dihydro-1,6-naphthyridin-6(5H)-yl)-5,6,7,8-
tetrahydroquinazoline
3-(2-fluoro-4-methylphenoxy)-6-(2,5,6-trimethylpyrimidin-4-yl)-5,6,7,8-tetrahydro-1,6-
naphthyridine
4-(4-(3-(1,3-dimethyl-1H-pyrazol-4-yl)-7,8-dihydro-1,6-naphthyridin-6(5H)-yl)-5,6-
dimethylpyrimidin-2-yl)morpholine
3-(1,3-dimethyl-1H-pyrazol-4-yl)-6-(5,6-dimethyl-2-(pyrrolidin-1-yl)pyrimidin-4-yl)-
5,6,7,8-tetrahydro-1,6-naphthyridine
N-cyclopropyl-4-(3-(1,3-dimethyl-1H-pyrazol-4-yl)-7,8-dihydro-1,6-naphthyridin-6(5H)-
yl)-5,6-dimethylpyrimidin-2-amine
4-(3-(1,3-dimethyl-1H-pyrazol-4-yl)-7,8-dihydro-1,6-naphthyridin-6(5H)-yl)-5,6-dimethyl-
N-(tetrahydrofuran-3-yl)pyrimidin-2-amine
1-((4-(3-(1,3-dimethyl-1H-pyrazol-4-yl)-7,8-dihydro-1,6-naphthyridin-6(5H)-yl)-5,6-
dimethylpyrimidin-2-yl)amino)-2-methylpropan-2-ol
3-(1,3-dimethyl-1H-pyrazol-4-yl)-6-(5,6-dimethyl-2-(4-methylpiperazin-1-yl)pyrimidin-4-
yl)-5,6,7,8-tetrahydro-1,6-naphthyridine
6-(5,6-dimethyl-2-morpholinopyrimidin-4-yl)-N-(1-methyl-1H-pyrazol-5-yl)-5,6,7,8-
tetrahydro-1,6-naphthyridin-3-amine
6-(5,6-dimethyl-2-(pyrrolidin-1-yl)pyrimidin-4-yl)-N-(1-methyl-1H-pyrazol-5-yl)-5,6,7,8-
tetrahydro-1,6-naphthyridin-3-amine
6-(2-(cyclopropylamino)-5,6-dimethylpyrimidin-4-yl)-N-(1-methyl-1H-pyrazol-5-yl)-
5,6,7,8-tetrahydro-1,6-naphthyridin-3-amine
6-(5,6-dimethyl-2-((tetrahydrofuran-3-yl)amino)pyrimidin-4-yl)-N-(1-methyl-1H-pyrazol-
5-yl)-5,6,7,8-tetrahydro-1,6-naphthyridin-3-amine
1-((4,5-dimethyl-6-(3-((1-methyl-1H-pyrazol-5-yl)amino)-7,8-dihydro-1,6-naphthyridin-
6(5H)-yl)pyrimidin-2-yl)amino)-2-methylpropan-2-ol
2-chloro-4-(3-(trifluoromethyl)-7,8-dihydro-1,6-naphthyridin-6(5H)-yl)-5,7-
dihydrofuro[3,4-d]pyrimidine
2-chloro-4-(3-(6,7-dihydropyrazolo[1,5-a]pyrimidin-4(5H)-yl)-7,8-dihydro-1,6-
naphthyridin-6(5H)-yl)-5,7-dihydrofuro[3,4-d]pyrimidine
1-(6-(2-chloro-5,7-dihydrofuro[3,4-d]pyrimidin-4-yl)-5,6,7,8-tetrahydro-1,6-naphthyridin-
3-yl)-2,3-dihydro-1H-pyrido[2,3-b][1,4]oxazine
(R)-2-chloro-4-(3-(2-methylmorpholino)-7,8-dihydro-1,6-naphthyridin-6(5H)-yl)-5,7-
dihydrofuro[3,4-d]pyrimidine
2-chloro-4-(3-(1,3-dimethyl-1H-pyrazol-4-yl)-7,8-dihydro-1,6-naphthyridin-6(5H)-yl)-5,7-
dihydrofuro[3,4-d]pyrimidine
2-chloro-4-(3-(1-cyclopropyl-1H-pyrazol-4-yl)-7,8-dihydro-1,6-naphthyridin-6(5H)-yl)-
5,7-dihydrofuro[3,4-d]pyrimidine
2-chloro-4-(3-cyclopropyl-7,8-dihydro-1,6-naphthyridin-6(5H)-yl)-5,7-dihydrofuro[3,4-
d]pyrimidine
6-(2-chloro-5,7-dihydrofuro[3,4-d]pyrimidin-4-yl)-N-(1-methyl-1H-pyrazol-5-yl)-5,6,7,8-
tetrahydro-1,6-naphthyridin-3-amine
6-(2-chloro-5,7-dihydrofuro[3,4-d]pyrimidin-4-yl)-N-(2-fluoropyridin-3-yl)-5,6,7,8-
tetrahydro-1,6-naphthyridin-3-amine
2-chloro-4-(3-(2-fluorophenoxy)-7,8-dihydro-1,6-naphthyridin-6(5H)-yl)-5,7-
dihydrofuro[3,4-d]pyrimidine
6-(3-(isothiazol-5-yl)-7,8-dihydro-1,6-naphthyridin-6(5H)-yl)-5-methylnicotinonitrile
6-(3-(isothiazol-5-yl)-7,8-dihydro-1,6-naphthyridin-6(5H)-yl)-4,5-dimethylpyridazine-3-
carbonitrile
4,5-dimethyl-6-(3-(2-methylthiazol-5-yl)-7,8-dihydro-1,6-naphthyridin-6(5H)-
yl)pyridazine-3-carbonitrile
6-(3-(2,4-dimethylthiazol-5-yl)-7,8-dihydro-1,6-naphthyridin-6(5H)-yl)-4,5-
dimethylpyridazine-3-carbonitrile
6-(3-(1,3-dimethyl-1H-pyrazol-4-yl)-7,8-dihydro-1,6-naphthyridin-6(5H)-yl)-4,5-
dimethylpyridazine-3-carbonitrile
4,5-dimethyl-6-(3-(1,3,5-trimethyl-1H-pyrazol-4-yl)-7,8-dihydro-1,6-naphthyridin-6(5H)-
yl)pyridazine-3-carbonitrile
6-(3-(3,5-dimethylisoxazol-4-yl)-7,8-dihydro-1,6-naphthyridin-6(5H)-yl)-4,5-
dimethylpyridazine-3-carbonitrile
4,5-dimethyl-6-(3-(6-methylpyridin-3-yl)-7,8-dihydro-1,6-naphthyridin-6(5H)-
yl)pyridazine-3-carbonitrile
6-(3-(6-methoxypyridin-3-yl)-7,8-dihydro-1,6-naphthyridin-6(5H)-yl)-4,5-
dimethylpyridazine-3-carbonitrile
6-(3-(4-fluorophenoxy)-7,8-dihydro-1,6-naphthyridin-6(5H)-yl)-4,5-dimethylpyridazine-3-
carbonitrile
6-(3-(6-cyclopropylpyridin-3-yl)-7,8-dihydro-1,6-naphthyridin-6(5H)-yl)-4,5-
dimethylpyridazine-3-carbonitrile
6-(3-((6-methoxypyridin-3-yl)oxy)-7,8-dihydro-1,6-naphthyridin-6(5H)-yl)-4,5-
dimethylpyridazine-3-carbonitrile
4,5-dimethyl-6-(3-(trifluoromethyl)-7,8-dihydro-1,6-naphthyridin-6(5H)-yl)pyridazine-3-
carbonitrile
5-methyl-6-(3-(trifluoromethyl)-7,8-dihydro-1,6-naphthyridin-6(5H)-yl)pyridazine-3-
carbonitrile
5-methyl-6-(3-methyl-7,8-dihydro-1,6-naphthyridin-6(5H)-yl)pyridazine-3-carbonitrile
5-methyl-6-(3-((1-methyl-1H-pyrazol-5-yl)amino)-7,8-dihydro-1,6-naphthyridin-6(5H)-
yl)nicotinonitrile
6-(3-(difluoromethyl)-7,8-dihydro-1,6-naphthyridin-6(5H)-yl)-5-methylnicotinonitrile
6-(3-(difluoromethyl)-7,8-dihydro-1,6-naphthyridin-6(5H)-yl)-4,5-dimethylpyridazine-3-
carbonitrile
6-(3-(difluoromethyl)-7,8-dihydro-1,6-naphthyridin-6(5H)-yl)-5-methylpyridazine-3-
carbonitrile
6-(3-(2,4-difluorophenoxy)-7,8-dihydro-1,6-naphthyridin-6(5H)-yl)-5-methylnicotinonitrile
5-methyl-6-(3-((1-methyl-1H-pyrazol-5-yl)amino)-7,8-dihydro-1,6-naphthyridin-6(5H)-
yl)pyridazine-3-carbonitrile
6-(3-(6-(dimethylamino)pyridin-3-yl)-7,8-dihydro-1,6-naphthyridin-6(5H)-yl)-4,5-
dimethylpyridazine-3-carbonitrile
6-(3-((1-isopentyl-3-methyl-1H-pyrazol-5-yl)amino)-7,8-dihydro-1,6-naphthyridin-6(5H)-
yl)-5-methylnicotinonitrile
6-(3-(6,7-dihydropyrazolo[1,5-a]pyrimidin-4(5H)-yl)-7,8-dihydro-1,6-naphthyridin-6(5H)-
yl)-5-methylnicotinonitrile
6-(3-((1-butyl-3-methyl-1H-pyrazol-5-yl)amino)-7,8-dihydro-1,6-naphthyridin-6(5H)-yl)-5-
methylnicotinonitrile
6-(3-((1-isopropyl-3-methyl-1H-pyrazol-5-yl)amino)-7,8-dihydro-1,6-naphthyridin-6(5H)-
yl)-5-methylnicotinonitrile
6-(3-cyclopropyl-7,8-dihydro-1,6-naphthyridin-6(5H)-yl)-4,5-dimethylpyridazine-3-
carbonitrile
6-(3-(6-(dimethylamino)pyridin-3-yl)-7,8-dihydro-1,6-naphthyridin-6(5H)-yl)-5-
methylnicotinonitrile
6-(3-(6-fluoropyridin-3-yl)-7,8-dihydro-1,6-naphthyridin-6(5H)-yl)-5-methylnicotinonitrile
6-(3-(6-fluoro-4-methylpyridin-3-yl)-7,8-dihydro-1,6-naphthyridin-6(5H)-yl)-4,5-
dimethylpyridazine-3-carbonitrile
6-(3-(6-cyclopropylpyridin-3-yl)-7,8-dihydro-1,6-naphthyridin-6(5H)-yl)-5-
methylpyridazine-3-carbonitrile
5-methyl-6-(3-(4-methylpyridin-3-yl)-7,8-dihydro-1,6-naphthyridin-6(5H)-yl)pyridazine-3-
carbonitrile
6-(3-(1,3-dimethyl-1H-pyrazol-4-yl)-7,8-dihydro-1,6-naphthyridin-6(5H)-yl)-5-
methylpyridazine-3-carbonitrile
6-(3-(1-(2-(dimethylamino)ethyl)-1H-pyrazol-4-yl)-7,8-dihydro-1,6-naphthyridin-6(5H)-
yl)-4,5-dimethylpyridazine-3-carbonitrile
4,5-dimethyl-6-(3-(1-(2-morpholinoethyl)-1H-pyrazol-4-yl)-7,8-dihydro-1,6-naphthyridin-
6(5H)-yl)pyridazine-3-carbonitrile
6-(3-(1-(difluoromethyl)-1H-pyrazol-4-yl)-7,8-dihydro-1,6-naphthyridin-6(5H)-yl)-4,5-
dimethylpyridazine-3-carbonitrile
6-(3-(1-(2-hydroxy-2-methylpropyl)-1H-pyrazol-4-yl)-7,8-dihydro-1,6-naphthyridin-6(5H)-
yl)-4,5-dimethylpyridazine-3-carbonitrile
4,5-dimethyl-6-(3-(6-(4-methylpiperazin-1-yl)pyridin-3-yl)-7,8-dihydro-1,6-naphthyridin-
6(5H)-yl)pyridazine-3-carbonitrile
6-(3-(6-(3-(2-hydroxypropan-2-yl)azetidin-1-yl)pyridin-3-yl)-7,8-dihydro-1,6-naphthyridin-
6(5H)-yl)-4,5-dimethylpyridazine-3-carbonitrile
6-(3-cyclopropyl-7,8-dihydro-1,6-naphthyridin-6(5H)-yl)-5-methylnicotinonitrile
5-methyl-6-(3-(methyl(1-methyl-1H-pyrazol-5-yl)amino)-7,8-dihydro-1,6-naphthyridin-
6(5H)-yl)nicotinonitrile
6-(3-(6-(2-oxa-6-azaspiro[3.3]heptan-6-yl)pyridin-3-yl)-7,8-dihydro-1,6-naphthyridin-
6(5H)-yl)-4,5-dimethylpyridazine-3-carbonitrile
6-(3-(6-(2-oxa-5-azabicyclo[2.2.1]heptan-5-yl)pyridin-3-yl)-7,8-dihydro-1,6-naphthyridin-
6(5H)-yl)-4,5-dimethylpyridazine-3-carbonitrile
6-(3-(6-fluoropyridin-3-yl)-7,8-dihydro-1,6-naphthyridin-6(5H)-yl)-4,5-
dimethylpyridazine-3-carbonitrile
6-(3-(6-(4-cyclopropylpiperazin-1-yl)pyridin-3-yl)-7,8-dihydro-1,6-naphthyridin-6(5H)-yl)-
4,5-dimethylpyridazine-3-carbonitrile
4-(6-(5-cyano-3-methylpyridin-2-yl)-5,6,7,8-tetrahydro-1,6-naphthyridin-3-
yl)picolinonitrile
6-(3-(2,5-difluoropyridin-4-yl)-7,8-dihydro-1,6-naphthyridin-6(5H)-yl)-5-
methylnicotinonitrile
6-(3-(2-methoxypyridin-4-yl)-7,8-dihydro-1,6-naphthyridin-6(5H)-yl)-5-
methylnicotinonitrile
6-(3-(2-cyclopropylpyridin-4-yl)-7,8-dihydro-1,6-naphthyridin-6(5H)-yl)-5-
methylnicotinonitrile
6-(3-(2-fluoropyridin-4-yl)-7,8-dihydro-1,6-naphthyridin-6(5H)-yl)-5-methylnicotinonitrile
6-(3-(5-chloro-2-methoxypyridin-4-yl)-7,8-dihydro-1,6-naphthyridin-6(5H)-yl)-5-
methylnicotinonitrile
6-(3-(6,7-dihydropyrazolo[1,5-a]pyrimidin-4(5H)-yl)-7,8-dihydro-1,6-naphthyridin-6(5H)-
yl)-4,5-dimethylpyridazine-3-carbonitrile
6-(3-(1H-imidazol-1-yl)-7,8-dihydro-1,6-naphthyridin-6(5H)-yl)-5-methylnicotinonitrile
6-(3-(1H-pyrazol-1-yl)-7,8-dihydro-1,6-naphthyridin-6(5H)-yl)-5-methylnicotinonitrile
5-methyl-6-(3-(5-methyl-1H-pyrazol-1-yl)-7,8-dihydro-1,6-naphthyridin-6(5H)-
yl)nicotinonitrile
6-(3-(3,5-dimethyl-1H-pyrazol-1-yl)-7,8-dihydro-1,6-naphthyridin-6(5H)-yl)-5-
methylnicotinonitrile
5-methyl-6-(8-methyl-3-(trifluoromethyl)-7,8-dihydro-1,6-naphthyridin-6(5H)-
yl)nicotinonitrile
4,5-dimethyl-6-(8-methyl-3-(trifluoromethyl)-7,8-dihydro-1,6-naphthyridin-6(5H)-
yl)pyridazine-3-carbonitrile
6-(3-(2-fluoro-4-methylphenoxy)-7,8-dihydro-1,6-naphthyridin-6(5H)-yl)-5-
methylnicotinonitrile
6-(3-(2,3-difluorophenoxy)-7,8-dihydro-1,6-naphthyridin-6(5H)-yl)-5-methylnicotinonitrile
6-(3-(2,5-difluorophenoxy)-7,8-dihydro-1,6-naphthyridin-6(5H)-yl)-5-methylnicotinonitrile
6-(3-(2,3-difluorophenoxy)-7,8-dihydro-1,6-naphthyridin-6(5H)-yl)-4,5-
dimethylpyridazine-3-carbonitrile
6-(3-(2,5-difluorophenoxy)-7,8-dihydro-1,6-naphthyridin-6(5H)-yl)-4,5-
dimethylpyridazine-3-carbonitrile
6-(3-(2,4-difluorophenoxy)-7,8-dihydro-1,6-naphthyridin-6(5H)-yl)-4,5-
dimethylpyridazine-3-carbonitrile
6-(3-(2,4-difluorophenoxy)-7,8-dihydro-1,6-naphthyridin-6(5H)-yl)-5-methylpyridazine-3-
carbonitrile
6-(3-(2,3-difluorophenoxy)-7,8-dihydro-1,6-naphthyridin-6(5H)-yl)-5-methylpyridazine-3-
carbonitrile
6-(3-(2,5-difluorophenoxy)-7,8-dihydro-1,6-naphthyridin-6(5H)-yl)-5-methylpyridazine-3-
carbonitrile
6-(3-(2-fluoro-4-methylphenoxy)-7,8-dihydro-1,6-naphthyridin-6(5H)-yl)-4,5-
dimethylpyridazine-3-carbonitrile
5-methyl-6-(3-(7-(trifluoromethyl)-6,7-dihydropyrazolo[1,5-a]pyrimidin-4(5H)-yl)-7,8-
dihydro-1,6-naphthyridin-6(5H)-yl)nicotinonitrile
6-(3-(6,7-dihydropyrazolo[1,5-a]pyrimidin-4(5H)-yl)-7,8-dihydro-1,6-naphthyridin-6(5H)-
yl)-5-methylpyridazine-3-carbonitrile
6-(3-(1-cyclopropyl-1H-pyrazol-4-yl)-7,8-dihydro-1,6-naphthyridin-6(5H)-yl)-5-
methylpyridazine-3-carbonitrile
6-(3-(3,4-dihydro-1,5-naphthyridin-1(2H)-yl)-7,8-dihydro-1,6-naphthyridin-6(5H)-yl)-5-
methylnicotinonitrile
4,5-dimethyl-6-(3-(2-methyl-6,7-dihydropyrazolo[1,5-a]pyrimidin-4(5H)-yl)-7,8-dihydro-
1,6-naphthyridin-6(5H)-yl)pyridazine-3-carbonitrile
4,5-dimethyl-6-(3-(7-methyl-6,7-dihydropyrazolo[1,5-a]pyrimidin-4(5H)-yl)-7,8-dihydro-
1,6-naphthyridin-6(5H)-yl)pyridazine-3-carbonitrile
6-(3-(2-fluorobenzyl)-7,8-dihydro-1,6-naphthyridin-6(5H)-yl)-5-methylnicotinonitrile
5-methyl-6-(7-methyl-3-(trifluoromethyl)-7,8-dihydro-1,6-naphthyridin-6(5H)-
yl)nicotinonitrile
4,5-dimethyl-6-(7-methyl-3-(trifluoromethyl)-7,8-dihydro-1,6-naphthyridin-6(5H)-
yl)pyridazine-3-carbonitrile
6-(3-(2-fluoro-5-methylphenoxy)-7,8-dihydro-1,6-naphthyridin-6(5H)-yl)-5-
methylnicotinonitrile
6-(3-(2-fluorobenzyl)-7,8-dihydro-1,6-naphthyridin-6(5H)-yl)-4,5-dimethylpyridazine-3-
carbonitrile
6-(3-(2,2-dimethylmorpholino)-7,8-dihydro-1,6-naphthyridin-6(5H)-yl)-5-
methylnicotinonitrile
2-methoxy-5-methyl-6-(3-(trifluoromethyl)-7,8-dihydro-1,6-naphthyridin-6(5H)-
yl)nicotinonitrile
5-methyl-2-(1-methyl-1H-pyrrol-2-yl)-6-(3-(trifluoromethyl)-7,8-dihydro-1,6-naphthyridin-
6(5H)-yl)nicotinonitrile
2-ethoxy-5-methyl-6-(3-(trifluoromethyl)-7,8-dihydro-1,6-naphthyridin-6(5H)-
yl)nicotinonitrile
4,5-dimethyl-6-(3-(6-methyl-6,7-dihydropyrazolo[1,5-a]pyrimidin-4(5H)-yl)-7,8-dihydro-
1,6-naphthyridin-6(5H)-yl)pyridazine-3-carbonitrile
(R)-5-methyl-6-(3-(2-phenylmorpholino)-7,8-dihydro-1,6-naphthyridin-6(5H)-
yl)nicotinonitrile
(R)-4,5-dimethyl-6-(3-(2-phenylmorpholino)-7,8-dihydro-1,6-naphthyridin-6(5H)-
yl)pyridazine-3-carbonitrile
6-(3-(2,2-dimethylmorpholino)-2-methyl-7,8-dihydro-1,6-naphthyridin-6(5H)-yl)-5-
methylnicotinonitrile
(S)-5-methyl-6-(3-(2-phenylmorpholino)-7,8-dihydro-1,6-naphthyridin-6(5H)-
yl)nicotinonitrile
(S)-4,5-dimethyl-6-(3-(2-phenylmorpholino)-7,8-dihydro-1,6-naphthyridin-6(5H)-
yl)pyridazine-3-carbonitrile
4,5-dimethyl-6-(3-(2-(pyridin-2-yl)morpholino)-7,8-dihydro-1,6-naphthyridin-6(5H)-
yl)pyridazine-3-carbonitrile
5-methyl-6-(3-(6-methyl-6,7-dihydropyrazolo[1,5-a]pyrimidin-4(5H)-yl)-7,8-dihydro-1,6-
naphthyridin-6(5H)-yl)pyridazine-3-carbonitrile
5-methyl-6-(3-(2-methyl-6,7-dihydropyrazolo[1,5-a]pyrimidin-4(5H)-yl)-7,8-dihydro-1,6-
naphthyridin-6(5H)-yl)pyridazine-3-carbonitrile
5-methyl-6-(3-(7-methyl-6,7-dihydropyrazolo[1,5-a]pyrimidin-4(5H)-yl)-7,8-dihydro-1,6-
naphthyridin-6(5H)-yl)pyridazine-3-carbonitrile
5-methyl-6-(3-(6-methyl-6,7-dihydropyrazolo[1,5-a]pyrimidin-4(5H)-yl)-7,8-dihydro-1,6-
naphthyridin-6(5H)-yl)nicotinonitrile
5-methyl-6-(3-(2-methyl-6,7-dihydropyrazolo[1,5-a]pyrimidin-4(5H)-yl)-7,8-dihydro-1,6-
naphthyridin-6(5H)-yl)nicotinonitrile
5-methyl-6-(3-(7-methyl-6,7-dihydropyrazolo[1,5-a]pyrimidin-4(5H)-yl)-7,8-dihydro-1,6-
naphthyridin-6(5H)-yl)nicotinonitrile
5-methyl-6-(3-(2-(pyridin-2-yl)morpholino)-7,8-dihydro-1,6-naphthyridin-6(5H)-
yl)nicotinonitrile
4,5-dimethyl-6-(3-(2-(pyridin-3-yl)morpholino)-7,8-dihydro-1,6-naphthyridin-6(5H)-
yl)pyridazine-3-carbonitrile
5-methyl-6-(3-(2-(pyridin-3-yl)morpholino)-7,8-dihydro-1,6-naphthyridin-6(5H)-
yl)nicotinonitrile
5-methyl-6-(3-(2-methylthiazol-5-yl)-7,8-dihydro-1,6-naphthyridin-6(5H)-yl)pyridazine-3-
carbonitrile
6-(3-(2,3-dihydro-1H-pyrido[2,3-b][1,4]oxazin-1-yl)-7,8-dihydro-1,6-naphthyridin-6(5H)-
yl)-5-methylpyridazine-3-carbonitrile
5-methyl-6-(3-(6-methylpyridin-3-yl)-7,8-dihydro-1,6-naphthyridin-6(5H)-yl)pyridazine-3-
carbonitrile
6-(3-cyclopropyl-7,8-dihydro-1,6-naphthyridin-6(5H)-yl)-5-methylpyridazine-3-carbonitrile
5-methyl-6-(3-(1-methyl-1H-pyrazol-5-yl)-7,8-dihydro-1,6-naphthyridin-6(5H)-
yl)pyridazine-3-carbonitrile
5-methyl-6-(3-(tetrahydrofuran-3-yl)-7,8-dihydro-1,6-naphthyridin-6(5H)-yl)nicotinonitrile
6-(3-(2-fluorophenoxy)-7,8-dihydro-1,6-naphthyridin-6(5H)-yl)-5-methylpyridazine-3-
carbonitrile
(R)-5-methyl-6-(3-(2-methylmorpholino)-7,8-dihydro-1,6-naphthyridin-6(5H)-
yl)pyridazine-3-carbonitrile
6-(3-(2,2-dimethylmorpholino)-7,8-dihydro-1,6-naphthyridin-6(5H)-yl)-5-
methylpyridazine-3-carbonitrile
6-(3-((2-fluoropyridin-3-yl)amino)-7,8-dihydro-1,6-naphthyridin-6(5H)-yl)-5-
methylpyridazine-3-carbonitrile
6-(3-(1H-pyrazol-1-yl)-7,8-dihydro-1,6-naphthyridin-6(5H)-yl)-5-methylpyridazine-3-
carbonitrile
6-(3-(3,5-dimethylisoxazol-4-yl)-7,8-dihydro-1,6-naphthyridin-6(5H)-yl)-5-
methylpyridazine-3-carbonitrile
6-(3-cyclobutyl-7,8-dihydro-1,6-naphthyridin-6(5H)-yl)-5-methylpyridazine-3-carbonitrile
6-(3-cyclobutyl-7,8-dihydro-1,6-naphthyridin-6(5H)-yl)-5-methylnicotinonitrile
5-methyl-6-(8-methyl-3-morpholino-7,8-dihydro-1,6-naphthyridin-6(5H)-yl)pyridazine-3-
carbonitrile
5-methyl-6-(8-methyl-3-morpholino-7,8-dihydro-1,6-naphthyridin-6(5H)-yl)nicotinonitrile
(R)-5-methyl-6-(8-methyl-3-morpholino-7,8-dihydro-1,6-naphthyridin-6(5H)-
yl)nicotinonitrile
(R)-5-methyl-6-(8-methyl-3-morpholino-7,8-dihydro-1,6-naphthyridin-6(5H)-
yl)pyridazine-3-carbonitrile
(S)-5-methyl-6-(8-methyl-3-morpholino-7,8-dihydro-1,6-naphthyridin-6(5H)-yl)pyridazine-
3-carbonitrile
(S)-5-methyl-6-(8-methyl-3-morpholino-7,8-dihydro-1,6-naphthyridin-6(5H)-
yl)nicotinonitrile
5-methyl-6-(3-(4-methyl-3-oxopiperazin-1-yl)-7,8-dihydro-1,6-naphthyridin-6(5H)-
yl)nicotinonitrile
5-methyl-6-(3-(4-methyl-3-oxopiperazin-1-yl)-7,8-dihydro-1,6-naphthyridin-6(5H)-
yl)pyridazine-3-carbonitrile
6-(3-(6,7-dihydroimidazo[1,2-a]pyrimidin-8(5H)-yl)-7,8-dihydro-1,6-naphthyridin-6(5H)-
yl)-5-methylnicotinonitrile
5-methyl-6-(3-(2-(trifluoromethyl)-6,7-dihydropyrazolo[1,5-a]pyrimidin-4(5H)-yl)-7,8-
dihydro-1,6-naphthyridin-6(5H)-yl)nicotinonitrile
5-methyl-6-(3-(2-(trifluoromethyl)-6,7-dihydropyrazolo[1,5-a]pyrimidin-4(5H)-yl)-7,8-
dihydro-1,6-naphthyridin-6(5H)-yl)pyridazine-3-carbonitrile
6-(3-(2-(tert-butyl)-6,7-dihydropyrazolo[1,5-a]pyrimidin-4(5H)-yl)-7,8-dihydro-1,6-
naphthyridin-6(5H)-yl)-5-methylnicotinonitrile
6-(3-(2-cyclopropyl-6,7-dihydropyrazolo[1,5-a]pyrimidin-4(5H)-yl)-7,8-dihydro-1,6-
naphthyridin-6(5H)-yl)-5-methylnicotinonitrile
6-(3-(6,7-dihydropyrazolo[1,5-a]pyrimidin-4(5H)-yl-5,5,6,6,7,7-d6)-7,8-dihydro-1,6-
naphthyridin-6(5H)-yl)-5-methylpyridazine-3-carbonitrile
6-(3-(6,7-dihydropyrazolo[1,5-a]pyrimidin-4(5H)-yl-5,5,6,6,7,7-d₆)-7,8-dihydro-1,6-
naphthyridin-6(5H)-yl)-5-methylnicotinonitrile
6-(3-(2-(tert-butyl)-6,7-dihydropyrazolo[1,5-a]pyrimidin-4(5H)-yl)-7,8-dihydro-1,6-
naphthyridin-6(5H)-yl)-5-methylpyridazine-3-carbonitrile
6-(3-(2-cyclopropyl-6,7-dihydropyrazolo[1,5-a]pyrimidin-4(5H)-yl)-7,8-dihydro-1,6-
naphthyridin-6(5H)-yl)-5-methylpyridazine-3-carbonitrile
5-methyl-6-(3-(4-methyl-2-oxopiperazin-1-yl)-7,8-dihydro-1,6-naphthyridin-6(5H)-
yl)nicotinonitrile
5-methyl-6-(3-(methyl-d₃)-7,8-dihydro-1,6-naphthyridin-6(5H)-yl)pyridazine-3-carbonitrile
5-methyl-6-(3-(2,2,2-trifluoroethyl)-7,8-dihydro-1,6-naphthyridin-6(5H)-yl)pyridazine-3-
carbonitrile
5-methyl-6-(3-(2,2,2-trifluoroethyl)-7,8-dihydro-1,6-naphthyridin-6(5H)-yl)nicotinonitrile
5-methyl-6-(3-(1,1,1-trifluoro-2-hydroxypropan-2-yl)-7,8-dihydro-1,6-naphthyridin-6(5H)-
yl)pyridazine-3-carbonitrile
4,5-dimethyl-6-(3-(1,1,1-trifluoro-2-hydroxypropan-2-yl)-7,8-dihydro-1,6-naphthyridin-
6(5H)-yl)pyridazine-3-carbonitrile
5-methyl-6-(3-(1,1,1-trifluoro-2-hydroxypropan-2-yl)-7,8-dihydro-1,6-naphthyridin-6(5H)-
yl)nicotinonitrile
5-methyl-6-(3-(2-phenoxyethoxy)-7,8-dihydro-1,6-naphthyridin-6(5H)-yl)nicotinonitrile
6-(3-(2-(4-fluorophenoxy)ethoxy)-7,8-dihydro-1,6-naphthyridin-6(5H)-yl)-5-
methylnicotinonitrile
6-(3-(2-(2-fluorophenoxy)ethoxy)-7,8-dihydro-1,6-naphthyridin-6(5H)-yl)-5-
methylnicotinonitrile
5-methyl-6-(3-(2-(o-tolyloxy)ethoxy)-7,8-dihydro-1,6-naphthyridin-6(5H)-yl)nicotinonitrile
5-methyl-6-(3-(2-(p-tolyloxy)ethoxy)-7,8-dihydro-1,6-naphthyridin-6(5H)-yl)nicotinonitrile
5-methyl-6-(3-(3-phenoxypropoxy)-7,8-dihydro-1,6-naphthyridin-6(5H)-yl)nicotinonitrile
6-(3-(2-(cyclopentyloxy)ethoxy)-7,8-dihydro-1,6-naphthyridin-6(5H)-yl)-5-
methylnicotinonitrile
6-(3-(2-methoxyethoxy)-7,8-dihydro-1,6-naphthyridin-6(5H)-yl)-5-methylnicotinonitrile
4,5-dimethyl-6-(3-(5-methylpyridin-3-yl)-7,8-dihydro-1,6-naphthyridin-6(5H)-
yl)pyridazine-3-carbonitrile
6-(3-(6-fluoro-4-methylpyridin-3-yl)-7,8-dihydro-1,6-naphthyridin-6(5H)-yl)-5-
methylnicotinonitrile
2-(3-(6-fluoro-4-methylpyridin-3-yl)-7,8-dihydro-1,6-naphthyridin-6(5H)-yl)-3-
methylisonicotinonitrile
5-methyl-6-(3-methyl-7,8-dihydro-1,6-naphthyridin-6(5H)-yl)-N-(pyridin-4-
yl)nicotinamide
5-methyl-6-(3-methyl-7,8-dihydro-1,6-naphthyridin-6(5H)-yl)-N-(thiazol-5-
ylmethyl)nicotinamide
5-methyl-6-(3-methyl-7,8-dihydro-1,6-naphthyridin-6(5H)-yl)-N-(pyridin-4-
ylmethyl)nicotinamide
5-methyl-6-(3-methyl-7,8-dihydro-1,6-naphthyridin-6(5H)-yl)-N-((3-methylpyridin-4-
yl)methyl)nicotinamide
5-methyl-N-(pyridin-4-yl)-6-(3-(trifluoromethyl)-7,8-dihydro-1,6-naphthyridin-6(5H)-
yl)nicotinamide
5-methyl-N-(thiazol-5-ylmethyl)-6-(3-(trifluoromethyl)-7,8-dihydro-1,6-naphthyridin-
6(5H)-yl)nicotinamide
5-methyl-N-(pyridin-4-ylmethyl)-6-(3-(trifluoromethyl)-7,8-dihydro-1,6-naphthyridin-
6(5H)-yl)nicotinamide
5-methyl-N-((3-methylpyridin-4-yl)methyl)-6-(3-(trifluoromethyl)-7,8-dihydro-1,6-
naphthyridin-6(5H)-yl)nicotinamide
5-methyl-N-(quinolin-3-ylmethyl)-6-(3-(trifluoromethyl)-7,8-dihydro-1,6-naphthyridin-
6(5H)-yl)nicotinamide
N-(benzo[b]thiophen-3-ylmethyl)-5-methyl-6-(3-(trifluoromethyl)-7,8-dihydro-1,6-
naphthyridin-6(5H)-yl)nicotinamide
5-methyl-6-(3-(trifluoromethyl)-7,8-dihydro-1,6-naphthyridin-6(5H)-yl)nicotinamide
5-methyl-6-(3-((1-methyl-1H-pyrazol-5-yl)amino)-7,8-dihydro-1,6-naphthyridin-6(5H)-yl)-
N-(thiazol-5-ylmethyl)nicotinamide
6-(3-(1,3-dimethyl-1H-pyrazol-4-yl)-7,8-dihydro-1,6-naphthyridin-6(5H)-yl)-5-methyl-N-
(pyridin-4-yl)nicotinamide
6-(3-(1,3-dimethyl-1H-pyrazol-4-yl)-7,8-dihydro-1,6-naphthyridin-6(5H)-yl)-5-methyl-N-
((3-methylpyridin-4-yl)methyl)nicotinamide
N-(benzo[b]thiophen-3-ylmethyl)-6-(3-(1,3-dimethyl-1H-pyrazol-4-yl)-7,8-dihydro-1,6-
naphthyridin-6(5H)-yl)-5-methylnicotinamide
N-(benzo[d]thiazol-2-ylmethyl)-6-(3-(1,3-dimethyl-1H-pyrazol-4-yl)-7,8-dihydro-1,6-
naphthyridin-6(5H)-yl)-5-methylnicotinamide
6-(3-(1,3-dimethyl-1H-pyrazol-4-yl)-7,8-dihydro-1,6-naphthyridin-6(5H)-yl)-N,5-dimethyl-
N-(pyridin-4-ylmethyl)nicotinamide
N-((2-chlorothiazol-5-yl)methyl)-6-(3-(1,3-dimethyl-1H-pyrazol-4-yl)-7,8-dihydro-1,6-
naphthyridin-6(5H)-yl)-5-methylnicotinamide
N-((6-cyanopyridin-3-yl)methyl)-6-(3-(1,3-dimethyl-1H-pyrazol-4-yl)-7,8-dihydro-1,6-
naphthyridin-6(5H)-yl)-5-methylnicotinamide
6-(3-(1,3-dimethyl-1H-pyrazol-4-yl)-7,8-dihydro-1,6-naphthyridin-6(5H)-yl)-5-methyl-N-
(pyridin-3-ylmethyl)nicotinamide
6-(3-(1,3-dimethyl-1H-pyrazol-4-yl)-7,8-dihydro-1,6-naphthyridin-6(5H)-yl)-5-methyl-N-
(pyridin-2-ylmethyl)nicotinamide
6-(3-(1,3-dimethyl-1H-pyrazol-4-yl)-7,8-dihydro-1,6-naphthyridin-6(5H)-yl)-5-methyl-N-
((tetrahydro-2H-pyran-4-yl)methyl)nicotinamide
6-(3-(1,3-dimethyl-1H-pyrazol-4-yl)-7,8-dihydro-1,6-naphthyridin-6(5H)-yl)-N-isobutyl-5-
methylnicotinamide
6-(3-(1,3-dimethyl-1H-pyrazol-4-yl)-7,8-dihydro-1,6-naphthyridin-6(5H)-yl)-N-(2-
hydroxy-2-methylpropyl)-5-methylnicotinamide
N-((2,6-dichloropyridin-3-yl)methyl)-6-(3-(1,3-dimethyl-1H-pyrazol-4-yl)-7,8-dihydro-1,6-
naphthyridin-6(5H)-yl)-5-methylnicotinamide
N-(benzo[b]thiophen-3-ylmethyl)-4,5-dimethyl-6-(3-(trifluoromethyl)-7,8-dihydro-1,6-
naphthyridin-6(5H)-yl)pyridazine-3-carboxamide
4,5-dimethyl-N-((3-methylpyridin-4-yl)methyl)-6-(3-(trifluoromethyl)-7,8-dihydro-1,6-
naphthyridin-6(5H)-yl)pyridazine-3-carboxamide
4,5-dimethyl-N-(thiazol-5-ylmethyl)-6-(3-(trifluoromethyl)-7,8-dihydro-1,6-naphthyridin-
6(5H)-yl)pyridazine-3-carboxamide
4,5-dimethyl-N-(pyridin-4-yl)-6-(3-(trifluoromethyl)-7,8-dihydro-1,6-naphthyridin-6(5H)-
yl)pyridazine-3-carboxamide
4,5-dimethyl-N-(pyridin-4-ylmethyl)-6-(3-(trifluoromethyl)-7,8-dihydro-1,6-naphthyridin-
6(5H)-yl)pyridazine-3-carboxamide
4,5-dimethyl-6-(3-methyl-7,8-dihydro-1,6-naphthyridin-6(5H)-yl)-N-(thiazol-5-
ylmethyl)pyridazine-3-carboxamide
4,5-dimethyl-6-(3-methyl-7,8-dihydro-1,6-naphthyridin-6(5H)-yl)-N-(pyridin-4-
yl)pyridazine-3-carboxamide
N-(benzo[b]thiophen-3-ylmethyl)-4,5-dimethyl-6-(3-methyl-7,8-dihydro-1,6-naphthyridin-
6(5H)-yl)pyridazine-3-carboxamide
4,5-dimethyl-6-(3-methyl-7,8-dihydro-1,6-naphthyridin-6(5H)-yl)-N-(quinolin-3-
ylmethyl)pyridazine-3-carboxamide
4,5-dimethyl-6-(3-methyl-7,8-dihydro-1,6-naphthyridin-6(5H)-yl)-N-((3-methylpyridin-4-
yl)methyl)pyridazine-3-carboxamide
5-methyl-6-(3-((1-methyl-1H-pyrazol-5-yl)amino)-7,8-dihydro-1,6-naphthyridin-6(5H)-yl)-
N-(pyridin-4-ylmethyl)nicotinamide
5-methyl-6-(3-((1-methyl-1H-pyrazol-5-yl)amino)-7,8-dihydro-1,6-naphthyridin-6(5H)-yl)-
N-((3-methylpyridin-4-yl)methyl)nicotinamide
6-(3-(1,3-dimethyl-1H-pyrazol-4-yl)-7,8-dihydro-1,6-naphthyridin-6(5H)-yl)-5-methyl-N-
(2-oxaspiro[3.3]heptan-6-yl)nicotinamide
N-(3,3-difluorocyclobutyl)-6-(3-(1,3-dimethyl-1H-pyrazol-4-yl)-7,8-dihydro-1,6-
naphthyridin-6(5H)-yl)-5-methylnicotinamide
6-(3-(1,3-dimethyl-1H-pyrazol-4-yl)-7,8-dihydro-1,6-naphthyridin-6(5H)-yl)-N-(3-
fluorobicyclo[1.1.1 ]pentan-1-yl)-5-methylnicotinamide
N-((6-(1H-imidazol-1-yl)pyridin-3-yl)methyl)-6-(3-(1,3-dimethyl-1H-pyrazol-4-yl)-7,8-
dihydro-1,6-naphthyridin-6(5H)-yl)-5-methylnicotinamide
N-([1,2,4]triazolo[1,5-a]pyridin-6-ylmethyl)-6-(3-(1,3-dimethyl-1H-pyrazol-4-yl)-7,8-
dihydro-1,6-naphthyridin-6(5H)-yl)-5-methylnicotinamide
6-(3-(1,3-dimethyl-1H-pyrazol-4-yl)-7,8-dihydro-1,6-naphthyridin-6(5H)-yl)-5-methyl-N-
((6-morpholinopyridin-3-yl)methyl)nicotinamide
N-((6-chloropyridin-3-yl)methyl)-6-(3-(1,3-dimethyl-1H-pyrazol-4-yl)-7,8-dihydro-1,6-
naphthyridin-6(5H)-yl)-5-methylnicotinamide
6-(3-(1,3-dimethyl-1H-pyrazol-4-yl)-7,8-dihydro-1,6-naphthyridin-6(5H)-yl)-5-methyl-N-
(pyridin-4-ylmethyl)nicotinamide
6-(3-(6-cyclopropylpyridin-3-yl)-7,8-dihydro-1,6-naphthyridin-6(5H)-yl)-5-methyl-N-
(pyridin-4-ylmethyl)nicotinamide
6-(3-(6-cyclopropylpyridin-3-yl)-7,8-dihydro-1,6-naphthyridin-6(5H)-yl)-5-methyl-N-
(thiazol-4-ylmethyl)nicotinamide
6-(3-(6-(dimethylamino)pyridin-3-yl)-7,8-dihydro-1,6-naphthyridin-6(5H)-yl)-5-methyl-N-
(thiazol-5-ylmethyl)nicotinamide
4,5-dimethyl-6-(3-methyl-7,8-dihydro-1,6-naphthyridin-6(5H)-yl)-N-(pyridin-4-
ylmethyl)pyridazine-3-carboxamide
6-(3-(1,3-dimethyl-1H-pyrazol-4-yl)-7,8-dihydro-1,6-naphthyridin-6(5H)-yl)-5-
(trifluoromethyl)nicotinamide
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(pyrrolidin-1-yl)methanone
6-(3-(6,7-dihydropyrazolo[1,5-a]pyrimidin-4(5H)-yl)-7,8-dihydro-1,6-naphthyridin-6(5H)-
yl)-5-methyl-N-(thiazol-5-ylmethyl)pyridazine-3-carboxamide
6-(3-(6,7-dihydropyrazolo[1,5-a]pyrimidin-4(5H)-yl)-7,8-dihydro-1,6-naphthyridin-6(5H)-
yl)-5-methyl-N-(pyridin-4-ylmethyl)pyridazine-3-carboxamide
6-(3-(6,7-dihydropyrazolo[1,5-a]pyrimidin-4(5H)-yl)-7,8-dihydro-1,6-naphthyridin-6(5H)-
yl)-5-methyl-N-((3-methylpyridin-4-yl)methyl)pyridazine-3-carboxamide
5-methyl-N-(thiazol-5-ylmethyl)-6-(3-(trifluoromethyl)-7,8-dihydro-1,6-naphthyridin-
6(5H)-yl)pyridazine-3-carboxamide
5-methyl-N-(pyridin-4-ylmethyl)-6-(3-(trifluoromethyl)-7,8-dihydro-1,6-naphthyridin-
6(5H)-yl)pyridazine-3-carboxamide
5-methyl-N-((3-methylpyridin-4-yl)methyl)-6-(3-(trifluoromethyl)-7,8-dihydro-1,6-
naphthyridin-6(5H)-yl)pyridazine-3-carboxamide
N-((3-methoxypyridin-4-yl)methyl)-5-methyl-6-(3-(trifluoromethyl)-7,8-dihydro-1,6-
naphthyridin-6(5H)-yl)pyridazine-3-carboxamide
N-((3-fluoropyridin-4-yl)methyl)-5-methyl-6-(3-(trifluoromethyl)-7,8-dihydro-1,6-
naphthyridin-6(5H)-yl)pyridazine-3-carboxamide
N-((3-chloropyridin-4-yl)methyl)-5-methyl-6-(3-(trifluoromethyl)-7,8-dihydro-1,6-
naphthyridin-6(5H)-yl)pyridazine-3-carboxamide
(3,4-dihydro-2,6-naphthyridin-2(1H)-yl)(5-methyl-6-(3-(trifluoromethyl)-7,8-dihydro-1,6-
naphthyridin-6(5H)-yl)pyridazin-3-yl)methanone
(3,4-dihydro-2,7-naphthyridin-2(1H)-yl)(5-methyl-6-(3-(trifluoromethyl)-7,8-dihydro-1,6-
naphthyridin-6(5H)-yl)pyridazin-3-yl)methanone
(5,8-dihydro-1,7-naphthyridin-7(6H)-yl)(5-methyl-6-(3-(trifluoromethyl)-7,8-dihydro-1,6-
naphthyridin-6(5H)-yl)pyridazin-3-yl)methanone
5-methyl-N-(5,6,7,8-tetrahydroquinolin-6-yl)-6-(3-(trifluoromethyl)-7,8-dihydro-1,6-
naphthyridin-6(5H)-yl)pyridazine-3-carboxamide
(5-methyl-6-(3-(trifluoromethyl)-7,8-dihydro-1,6-naphthyridin-6(5H)-yl)pyridazin-3-
yl)(pyrrolidin-1-yl)methanone
(6,7-dihydropyrazolo[1,5-a]pyrimidin-4(5H)-yl)(5-methyl-6-(3-(trifluoromethyl)-7,8-
dihydro-1,6-naphthyridin-6(5H)-yl)pyridazin-3-yl)methanone
(3-azabicyclo[3.1.0]hexan-3-yl)(5-methyl-6-(3-(trifluoromethyl)-7,8-dihydro-1,6-
naphthyridin-6(5H)-yl)pyridazin-3-yl)methanone
5-methyl-N-(pyridin-4-ylmethyl-d₂)-6-(3-(trifluoromethyl)-7,8-dihydro-1,6-naphthyridin-
6(5H)-yl)pyridazine-3-carboxamide
N,5-dimethyl-N-(pyridin-4-ylmethyl)-6-(3-(trifluoromethyl)-7,8-dihydro-1,6-naphthyridin-
6(5H)-yl)pyridazine-3-carboxamide
5-methyl-N-(thiazol-4-ylmethyl-d₂)-6-(3-(trifluoromethyl)-7,8-dihydro-1,6-naphthyridin-
6(5H)-yl)pyridazine-3-carboxamide
6-(3-(6,7-dihydropyrazolo[1,5-a]pyrimidin-4(5H)-yl)-7,8-dihydro-1,6-naphthyridin-6(5H)-
yl)-5-methyl-N-(thiazol-4-ylmethyl-d₂)pyridazine-3-carboxamide
6-(3-(6,7-dihydropyrazolo[1,5-a]pyrimidin-4(5H)-yl)-7,8-dihydro-1,6-naphthyridin-6(5H)-
yl)-5-methyl-N-(pyridin-4-ylmethyl-d₂)pyridazine-3-carboxamide
N-(thiazol-5-ylmethyl)-6-(3-(trifluoromethyl)-7,8-dihydro-1,6-naphthyridin-6(5H)-
yl)pyridazine-3-carboxamide
N-(pyridin-4-ylmethyl)-6-(3-(trifluoromethyl)-7,8-dihydro-1,6-naphthyridin-6(5H)-
yl)pyridazine-3-carboxamide
(1,3-dihydro-2H-pyrrolo[3,4-c]pyridin-2-yl)(6-(3-(6,7-dihydropyrazolo[1,5-a]pyrimidin-
4(5H)-yl)-7,8-dihydro-1,6-naphthyridin-6(5H)-yl)-5-methylpyridazin-3-yl)methanone
(5,7-dihydro-6H-pyrrolo[3,4-b]pyridin-6-yl)(6-(3-(6,7-dihydropyrazolo[1,5-a]pyrimidin-
4(5H)-yl)-7,8-dihydro-1,6-naphthyridin-6(5H)-yl)-5-methylpyridazin-3-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(4-methylpiperazin-1-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(morpholino)methanone
5-methyl-6-(3-(1-methyl-1H-pyrazol-5-yl)-7,8-dihydro-1,6-naphthyridin-6(5H)-yl)-N-
(thiazol-5-ylmethyl)pyridazine-3-carboxamide
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-(pyridin-4-yl)azetidin-1-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-(dimethylamino)azetidin-1-yl)methanone
1-(6-(3-(6,7-dihydropyrazolo[1,5-a]pyrimidin-4(5H)-yl)-7,8-dihydro-1,6-naphthyridin-
6(5H)-yl)-5-methylpyridazine-3-carbonyl)-3-methylazetidine-3-carbonitrile
6-(3-(6,7-dihydropyrazolo[1,5-a]pyrimidin-4(5H)-yl)-7,8-dihydro-1,6-naphthyridin-6(5H)-
yl)-5-methyl-N-(1-(2,2,2-trifluoroethyl)azetidin-3-yl)pyridazine-3-carboxamide
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-(pyridin-4-yl)pyrrolidin-1-yl)methanone
(3-(difluoromethyl)azetidin-1-yl)(5-methyl-6-(3-(trifluoromethyl)-7,8-dihydro-1,6-
naphthyridin-6(5H)-yl)pyridazin-3-yl)methanone
N-(1-cyanocyclopropyl)-5-methyl-6-(3-(trifluoromethyl)-7,8-dihydro-1,6-naphthyridin-
6(5H)-yl)pyridazine-3-carboxamide
(3-(difluoromethyl)azetidin-1-yl)(6-(3-(6,7-dihydropyrazolo[1,5-a]pyrimidin-4(5H)-yl)-7,8-
dihydro-1,6-naphthyridin-6(5H)-yl)-5-methylpyridazin-3-yl)methanone
N-(1-cyanocyclopropyl)-6-(3-(6,7-dihydropyrazolo[1,5-a]pyrimidin-4(5H)-yl)-7,8-dihydro-
1,6-naphthyridin-6(5H)-yl)-5-methylpyridazine-3-carboxamide
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-(pyridin-3-yl)azetidin-1-yl)methanone
(3-chloroazetidin-1-yl)(6-(3-(6,7-dihydropyrazolo[1,5-a]pyrimidin-4(5H)-yl)-7,8-dihydro-
1,6-naphthyridin-6(5H)-yl)-5-methylpyridazin-3-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-(2-hydroxypropan-2-yl)azetidin-1-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(2,2-dimethylpyrrolidin-1-yl)methanone
(6-(3-(1H-pyrazol-1-yl)-7,8-dihydro-1,6-naphthyridin-6(5H)-yl)-5-methylpyridazin-3-
yl)(pyrrolidin-1-yl)methanone
(6-(3-(1H-pyrazol-1-yl)-7,8-dihydro-1,6-naphthyridin-6(5H)-yl)-5-methylpyridazin-3-
yl)(azetidin-1-yl)methanone
(6-(3-(1H-pyrazol-1-yl)-7,8-dihydro-1,6-naphthyridin-6(5H)-yl)-5-methylpyridazin-3-yl)(3-
chloroazetidin-1-yl)methanone
(6-(3-(1H-pyrazol-1-yl)-7,8-dihydro-1,6-naphthyridin-6(5H)-yl)-5-methylpyridazin-3-
yl)(3,3-difluoroazetidin-1-yl)methanone
(6-(3-(1H-pyrazol-1-yl)-7,8-dihydro-1,6-naphthyridin-6(5H)-yl)-5-methylpyridazin-3-yl)(3-
hydroxy-3-methylazetidin-1-yl)methanone
(6-(3-(1H-pyrazol-1-yl)-7,8-dihydro-1,6-naphthyridin-6(5H)-yl)-5-methylpyridazin-3-yl)(3-
(pyridin-3-yl)azetidin-1-yl)methanone
(R)-(5-methyl-6-(3-(2-methylmorpholino)-7,8-dihydro-1,6-naphthyridin-6(5H)-
yl)pyridazin-3-yl)(pyrrolidin-1-yl)methanone
(R)-azetidin-1-yl(5-methyl-6-(3-(2-methylmorpholino)-7,8-dihydro-1,6-naphthyridin-
6(5H)-yl)pyridazin-3-yl)methanone
(R)-(3-chloroazetidin-1-yl)(5-methyl-6-(3-(2-methylmorpholino)-7,8-dihydro-1,6-
naphthyridin-6(5H)-yl)pyridazin-3-yl)methanone
(R)-(3,3-difluoroazetidin-1-yl)(5-methyl-6-(3-(2-methylmorpholino)-7,8-dihydro-1,6-
naphthyridin-6(5H)-yl)pyridazin-3-yl)methanone
(R)-(3-hydroxy-3-methylazetidin-1-yl)(5-methyl-6-(3-(2-methylmorpholino)-7,8-dihydro-
1,6-naphthyridin-6(5H)-yl)pyridazin-3-yl)methanone
(R)-(5-methyl-6-(3-(2-methylmorpholino)-7,8-dihydro-1,6-naphthyridin-6(5H)-
yl)pyridazin-3-yl)(3-(pyridin-3-yl)azetidin-1-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(2-oxa-6-azaspiro[3.3]heptan-6-yl)methanone
6-(3-(6,7-dihydropyrazolo[1,5-a]pyrimidin-4(5H)-yl)-7,8-dihydro-1,6-naphthyridin-6(5H)-
yl)-5-methyl-N-(1-(pyridin-2-yl)cyclopropyl)pyridazine-3-carboxamide
(3,3-difluoroazetidin-1-yl)(6-(3-(6,7-dihydropyrazolo[1,5-a]pyrimidin-4(5H)-yl)-7,8-
dihydro-1,6-naphthyridin-6(5H)-yl)-5-methylpyridazin-3-yl)methanone
azetidin-1-yl(6-(3-(6,7-dihydropyrazolo[1,5-a]pyrimidin-4(5H)-yl)-7,8-dihydro-1,6-
naphthyridin-6(5H)-yl)-5-methylpyridazin-3-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-fluoro-3-methylazetidin-1-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-hydroxy-3-methylazetidin-1-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-methoxyazetidin-1-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-methylazetidin-1-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(2-methylazetidin-1-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-fluoroazetidin-1-yl)methanone
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-methoxy-3-methylazetidin-1-yl)methanone
6-(3-(6,7-dihydropyrazolo[1,5-a]pyrimidin-4(5H)-yl)-7,8-dihydro-1,6-naphthyridin-6(5H)-
yl)-5-methyl-N-(1-(pyridin-3-yl)cyclopropyl)pyridazine-3-carboxamide
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(3-(hydroxymethyl)azetidin-1-yl)methanone
(6,6-difluoro-2-azaspiro[3.3]heptan-2-yl)(6-(3-(6,7-dihydropyrazolo[1,5-a]pyrimidin-
4(5H)-yl)-7,8-dihydro-1,6-naphthyridin-6(5H)-yl)-5-methylpyridazin-3-yl)methanone
6-(3-(6,7-dihydropyrazolo[1,5-a]pyrimidin-4(5H)-yl)-7,8-dihydro-1,6-naphthyridin-6(5H)-
yl)-N,N,5-trimethylpyridazine-3-carboxamide
(6-(3-(6,7-dihydropyrazolo[1,5-a]pyrimidin-4(5H)-yl)-7,8-dihydro-1,6-naphthyridin-6(5H)-
yl)-5-methylpyridazin-3-yl)(2-phenylazetidin-1-yl)methanone
(3-(difluoromethyl)azetidin-1-yl)(5-methyl-6-(3-((1-methyl-1H-pyrazol-5-yl)amino)-7,8-
dihydro-1,6-naphthyridin-6(5H)-yl)pyridazin-3-yl)methanone
(3-fluoroazetidin-1-yl)(5-methyl-6-(3-((1-methyl-1H-pyrazol-5-yl)amino)-7,8-dihydro-1,6-
naphthyridin-6(5H)-yl)pyridazin-3-yl)methanone
(3-fluoro-3-methylazetidin-1-yl)(5-methyl-6-(3-((1-methyl-1H-pyrazol-5-yl)amino)-7,8-
dihydro-1,6-naphthyridin-6(5H)-yl)pyridazin-3-yl)methanone
(5-methyl-6-(3-((1-methyl-1H-pyrazol-5-yl)amino)-7,8-dihydro-1,6-naphthyridin-6(5H)-
yl)pyridazin-3-yl)(2-oxa-6-azaspiro[3.3]heptan-6-yl)methanone
(3-(difluoromethyl)azetidin-1-yl)(5-methyl-6-(3-(1-methyl-1H-pyrazol-5-yl)-7,8-dihydro-
1,6-naphthyridin-6(5H)-yl)pyridazin-3-yl)methanone
(3-fluoroazetidin-1-yl)(5-methyl-6-(3-(1-methyl-1H-pyrazol-5-yl)-7,8-dihydro-1,6-
naphthyridin-6(5H)-yl)pyridazin-3-yl)methanone
(3-fluoro-3-methylazetidin-1-yl)(5-methyl-6-(3-(1-methyl-1H-pyrazol-5-yl)-7,8-dihydro-
1,6-naphthyridin-6(5H)-yl)pyridazin-3-yl)methanone
(5-methyl-6-(3-(1-methyl-1H-pyrazol-5-yl)-7,8-dihydro-1,6-naphthyridin-6(5H)-
yl)pyridazin-3-yl)(2-oxa-6-azaspiro[3.3]heptan-6-yl)methanone
(3,3-difluoroazetidin-1-yl)(5-methyl-6-(3-((1-methyl-1H-pyrazol-5-yl)amino)-7,8-dihydro-
1,6-naphthyridin-6(5H)-yl)pyridazin-3-yl)methanone
(3,3-difluoroazetidin-1-yl)(5-methyl-6-(3-(1-methyl-1H-pyrazol-5-yl)-7,8-dihydro-1,6-
naphthyridin-6(5H)-yl)pyridazin-3-yl)methanone
(6-(3-(1H-pyrazol-1-yl)-7,8-dihydro-1,6-naphthyridin-6(5H)-yl)-5-methylpyridazin-3-yl)(3-
fluoroazetidin-1-yl)methanone
(6-(3-(1H-pyrazol-1-yl)-7,8-dihydro-1,6-naphthyridin-6(5H)-yl)-5-methylpyridazin-3-yl)(3-
fluoro-3-methylazetidin-1-yl)methanone
(6-(3-(1H-pyrazol-1-yl)-7,8-dihydro-1,6-naphthyridin-6(5H)-yl)-5-methylpyridazin-3-
yl)(6,6-difluoro-2-azaspiro[3.3]heptan-2-yl)methanone
(6-(3-(1H-pyrazol-1-yl)-7,8-dihydro-1,6-naphthyridin-6(5H)-yl)-5-methylpyridazin-3-yl)(2-
oxa-6-azaspiro[3.3]heptan-6-yl)methanone
(R)-(3-fluoroazetidin-1-yl)(5-methyl-6-(3-(2-methylmorpholino)-7,8-dihydro-1,6-
naphthyridin-6(5H)-yl)pyridazin-3-yl)methanone
(R)-(3-fluoro-3-methylazetidin-1-yl)(5-methyl-6-(3-(2-methylmorpholino)-7,8-dihydro-1,6-
naphthyridin-6(5H)-yl)pyridazin-3-yl)methanone
(R)-(6,6-difluoro-2-azaspiro[3.3]heptan-2-yl)(5-methyl-6-(3-(2-methylmorpholino)-7,8-
dihydro-1,6-naphthyridin-6(5H)-yl)pyridazin-3-yl)methanone
(R)-(5-methyl-6-(3-(2-methylmorpholino)-7,8-dihydro-1,6-naphthyridin-6(5H)-
yl)pyridazin-3-yl)(2-oxa-6-azaspiro[3.3]heptan-6-yl)methanone
(3,3-difluoroazetidin-1-yl)(5-methyl-6-(3-(methyl-d₃)-7,8-dihydro-1,6-naphthyridin-6(5H)-
yl)pyridazin-3-yl)methanone
(5-methyl-6-(3-(methyl-d₃)-7,8-dihydro-1,6-naphthyridin-6(5H)-yl)pyridazin-3-yl)(2-oxa-6-
azaspiro[3.3]heptan-6-yl)methanone
(5-methyl-6-(3-(methyl-d₃)-7,8-dihydro-1,6-naphthyridin-6(5H)-yl)pyridazin-3-
yl)(pyrrolidin-1-yl)methanone
5-methyl-6-(3-(methyl-d₃)-7,8-dihydro-1,6-naphthyridin-6(5H)-yl)-N-(pyridin-4-
ylmethyl)pyridazine-3-carboxamide
5-methyl-6-(3-(methyl-d₃)-7,8-dihydro-1,6-naphthyridin-6(5H)-yl)-N-(1-(pyridin-3-
yl)cyclopropyl)pyridazine-3-carboxamide
5-methyl-N-(pyridin-4-ylmethyl)-6-(3-(2,2,2-trifluoroethyl)-7,8-dihydro-1,6-naphthyridin-
6(5H)-yl)pyridazine-3-carboxamide
(5-methyl-6-(3-(2,2,2-trifluoroethyl)-7,8-dihydro-1,6-naphthyridin-6(5H)-yl)pyridazin-3-
yl)(pyrrolidin-1-yl)methanone
(3,3-difluoroazetidin-1-yl)(5-methyl-6-(3-(2,2,2-trifluoroethyl)-7,8-dihydro-1,6-
naphthyridin-6(5H)-yl)pyridazin-3-yl)methanone
(3,3-bis(hydroxymethyl)azetidin-1-yl)(5-methyl-6-(3-(2,2,2-trifluoroethyl)-7,8-dihydro-1,6-
naphthyridin-6(5H)-yl)pyridazin-3-yl)methanone
5-methyl-N-(1-(pyridin-3-yl)cyclopropyl)-6-(3-(2,2,2-trifluoroethyl)-7,8-dihydro-1,6-
naphthyridin-6(5H)-yl)pyridazine-3-carboxamide
(3-fluoroazetidin-1-yl)(5-methyl-6-(3-(2,2,2-trifluoroethyl)-7,8-dihydro-1,6-naphthyridin-
6(5H)-yl)pyridazin-3-yl)methanone
(5-methyl-6-(3-(2,2,2-trifluoroethyl)-7,8-dihydro-1,6-naphthyridin-6(5H)-yl)pyridazin-3-
yl)(morpholino)methanone
(3-hydroxy-3-methylazetidin-1-yl)(5-methyl-6-(3-(2,2,2-trifluoroethyl)-7,8-dihydro-1,6-
naphthyridin-6(5H)-yl)pyridazin-3-yl)methanone
(3-methoxyazetidin-1-yl)(5-methyl-6-(3-(2,2,2-trifluoroethyl)-7,8-dihydro-1,6-
naphthyridin-6(5H)-yl)pyridazin-3-yl)methanone
5-methyl-N-(thiazol-5-ylmethyl)-6-(3-(2,2,2-trifluoroethyl)-7,8-dihydro-1,6-naphthyridin-
6(5H)-yl)pyridazine-3-carboxamide
4-benzyl-7-bromo-8-(3-(trifluoromethyl)-7,8-dihydro-1,6-naphthyridin-6(5H)-yl)-3,4-
dihydropyrido[3,2-f][1,4]oxazepin-5(2H)-one
4-benzyl-7-methyl-8-(3-(trifluoromethyl)-7,8-dihydro-1,6-naphthyridin-6(5H)-yl)-3,4-
dihydropyrido[3,2-f][1,4]oxazepin-5(2H)-one
6-(3-(6-fluoro-4-methylpyridin-3-yl)-7,8-dihydro-1,6-naphthyridin-6(5H)-yl)-5-
methylpyridazine-3-carbonitrile
6-(3-bromo-7,8-dihydro-1,6-naphthyridin-6(5H)-yl)-5-methylnicotinonitrile
6-(3-(4,5-difluoro-2-methylphenyl)-7,8-dihydro-1,6-naphthyridin-6(5H)-yl)-5-
methylnicotinonitrile
6-(3-(4,5-difluoro-2-methylphenyl)-7,8-dihydro-1,6-naphthyridin-6(5H)-yl)-5-
methylpyridazine-3-carbonitrile
6-(3-(3,5-difluoro-2-methylphenyl)-7,8-dihydro-1,6-naphthyridin-6(5H)-yl)-5-
methylnicotinonitrile
6-(3-((3-fluoropyridin-4-yl)amino)-7,8-dihydro-1,6-naphthyridin-6(5H)-yl)-5-
methylpyridazine-3-carbonitrile
6-(3-((3-fluoropyridin-4-yl)amino)-7,8-dihydro-1,6-naphthyridin-6(5H)-yl)-5-
methylnicotinonitrile
6-(3-((5-fluoro-2-methylpyridin-4-yl)amino)-7,8-dihydro-1,6-naphthyridin-6(5H)-yl-
5,5,7,7-d₄)-4,5-dimethylpyridazine-3-carbonitrile
6-(3-((5-fluoro-2-methylpyridin-4-yl)amino)-7,8-dihydro-1,6-naphthyridin-6(5H)-yl-
5,5,7,7-d₄)-5-methylnicotinonitrile
5-methyl-6-(3-((2-methylpyridin-3-yl)amino)-7,8-dihydro-1,6-naphthyridin-6(5H)-yl-
5,5,7,7-d₄)nicotinonitrile
6-(3-((5-fluoro-2-methylpyridin-4-yl)amino)-7,8-dihydro-1,6-naphthyridin-6(5H)-yl-
5,5,7,7-d₄)-5-methylpyridazine-3-carbonitrile
5-methyl-6-(3-((2-methylpyridin-3-yl)amino)-7,8-dihydro-1,6-naphthyridin-6(5H)-yl-
5,5,7,7-d₄)pyridazine-3-carbonitrile
5-methyl-6-(2-methyl-7,8-dihydro-1,6-naphthyridin-6(5H)-yl)pyridazine-3-carbonitrile
6-(3-((3-fluoropyridin-4-yl)amino)-7,8-dihydro-1,6-naphthyridin-6(5H)-yl-5,5,7,7-d₄)-5-
methylpyridazine-3-carbonitrile
5-methyl-6-(3-((3-methylpyridin-4-yl)amino)-7,8-dihydro-1,6-naphthyridin-6(5H)-yl-
5,5,7,7-d₄)pyridazine-3-carbonitrile
6-(3-((3-fluoropyridin-4-yl)amino)-7,8-dihydro-1,6-naphthyridin-6(5H)-yl-5,5,7,7-d₄)-4,5-
dimethylpyridazine-3-carbonitrile
4,5-dimethyl-6-(3-((3-methylpyridin-4-yl)amino)-7,8-dihydro-1,6-naphthyridin-6(5H)-yl-
5,5,7,7-d₄)pyridazine-3-carbonitrile
5-methyl-6-(3-((3-methylpyridin-4-yl)amino)-7,8-dihydro-1,6-naphthyridin-6(5H)-yl-
5,5,7,7-d₄)nicotinonitrile
6-(3-((2-fluorophenyl)amino)-7,8-dihydro-1,6-naphthyridin-6(5H)-yl-5,5,7,7-d₄)-5-
methylpyridazine-3-carbonitrile
6-(3-((2-fluorophenyl)amino)-7,8-dihydro-1,6-naphthyridin-6(5H)-yl-5,5,7,7-d₄)-5-
methylnicotinonitrile
6-(3-((3-fluoropyridin-4-yl)amino)-7,8-dihydro-1,6-naphthyridin-6(5H)-yl)-4,5-
dimethylpyridazine-3-carbonitrile
6-(3-((5-fluoro-2-methylpyridin-4-yl)amino)-7,8-dihydro-1,6-naphthyridin-6(5H)-yl)-4,5-
dimethylpyridazine-3-carbonitrile
6-(3-((5-fluoro-2-methylpyridin-4-yl)amino)-7,8-dihydro-1,6-naphthyridin-6(5H)-yl)-5-
methylpyridazine-3-carbonitrile
6-(3-((5-fluoro-2-methylpyridin-4-yl)amino)-7,8-dihydro-1,6-naphthyridin-6(5H)-yl)-5-
methylnicotinonitrile
5-methyl-6-(3-(1-methyl-1H-pyrazol-5-yl)-7,8-dihydro-1,6-naphthyridin-6(5H)-yl-5,5,7,7-
d₄)pyridazine-3-carbonitrile
4,5-dimethyl-6-(3-((3-methylpyridin-4-yl)amino)-7,8-dihydro-1,6-naphthyridin-6(5H)-
yl)pyridazine-3-carbonitrile
5-methyl-6-(3-((3-methylpyridin-4-yl)amino)-7,8-dihydro-1,6-naphthyridin-6(5H)-
yl)pyridazine-3-carbonitrile
5-methyl-6-(3-((3-methylpyridin-4-yl)amino)-7,8-dihydro-1,6-naphthyridin-6(5H)-
yl)nicotinonitrile
6-(3-(3,5-dimethylisoxazol-4-yl)-7,8-dihydro-1,6-naphthyridin-6(5H)-yl-5,5,7,7-d₄)-5-
methylpyridazine-3-carbonitrile

or a pharmaceutically acceptable salt thereof.
E37. A pharmaceutical composition comprising the compound of any of E1-E36, or a pharmaceutically acceptable salt thereof, and a pharmaceutically acceptable carrier.
E38. A method for treating a neurological and/or psychiatric disorder associated with muscarinic acetylcholine receptor dysfunction in a mammal, comprising administering to the mammal a therapeutically effective amount of the compound of any of E1-E36, or pharmaceutically acceptable salt thereof, or the pharmaceutical composition of E37.
E39. The method of E38, wherein the disorder is associated with a mAChR M₄ dysfunction.
E40. The method of E38 or E39, wherein the disorder is a neurological and/or psychiatric disorder associated with mAChR M₄ dysfunction.
E41. The method of any of E38-E40, wherein the disorder is selected from Alzheimer's disease, schizophrenia, a sleep disorder, a pain disorder, and a cognitive disorder.
E42. The method of E41, wherein the disorder is Alzheimer's disease.
E43. The method of any of E38-E40, wherein the disorder is selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.
E44. A kit comprising the compound of any of E1-E36, or a pharmaceutically acceptable salt thereof, and one or more of: (a) at least one agent known to increase mAChR M₄ activity; (b) at least one agent known to decrease mAChR M₄ activity; (c) at least one agent known to treat a disorder associated with cholinergic activity; (d) instructions for treating a disorder associated with cholinergic activity; (e) instructions for treating a disorder associated with mAChR M₄ receptor activity; and (f) instructions for administering the compound in connection with cognitive or behavioral therapy.
E45. The compound of any of E1-E36, or a pharmaceutically acceptable salt thereof, or the pharmaceutical composition of E37, for use in the treatment of a neurological and/or psychiatric disorder associated with muscarinic acetylcholine receptor dysfunction in a mammal.
E46. The use of the compound of any of E1-E36, or a pharmaceutically acceptable salt thereof, or the pharmaceutical composition of E37 for the preparation of a medicament for the treatment of a neurological and/or psychiatric disorder associated with muscarinic acetylcholine receptor dysfunction in a mammal.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

It should be understood that the compound may possess tautomeric forms, as well as geometric isomers, and that these also constitute embodiments of the disclosure.

In the compounds of formula (I), and any subformulas, any “hydrogen” or “H,” whether explicitly recited or implicit in the structure, encompasses hydrogen isotopes ¹H (protium) and ²H (deuterium).

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ^{2H}, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e. ^{2H}, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

In some embodiments, R¹ is deuterium. In some embodiments, R² is deuterium. In some embodiments, R⁵ is deuterium.

a. Pharmaceutically Acceptable Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

b. General Synthesis

Compounds of formula (I) may be prepared by synthetic processes or by metabolic processes. Preparation of the compounds by metabolic processes includes those occurring in the human or animal body (in vivo) or processes occurring in vitro.

Abbreviations used in the schemes that follow include the following: ACN is acetonitrile; DCM is dichloromethane; DIEA is diisopropylethylamine; 1,4-diox is 1,4 dioxane; DMF is N,N-dimethylformamide; h is hours; HATU is 2-(7-aza-1H-benzotriazole-1-yl)-1,1,3,3-tetramethyluronium hexafluorophosphate; min is minutes; μW is microwave (referring to a microwave reactor); NMP is N-methylpyrrolidone; Pd₂(dba)₃ is tris(dibenzylideneacetone)dipalladium(0); Pd(dppf)Cl₂ is [1,1′-Bis(diphenylphosphino)ferrocene]palladium(II) dichloride; THE is tetrahydrofuran; Xantphos is 4,5-bis(diphenylphosphino)-9,9-dimethylxanthene.

Compounds of formula (I) may be synthesized as shown in Schemes 1-15.

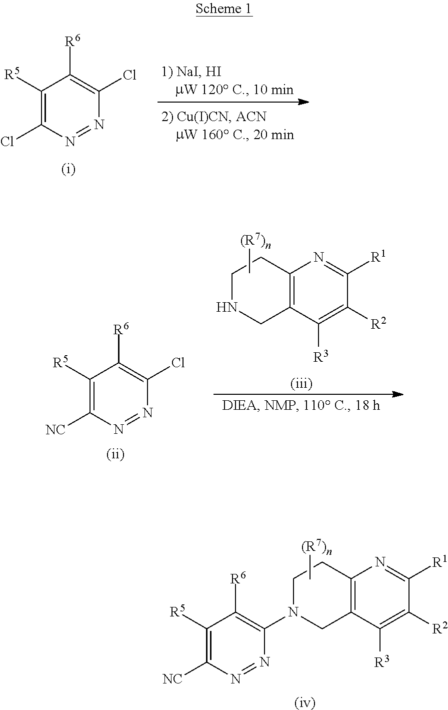

5,6,7,8-Tetrahydronaphthyridines (iv) (e.g., R² is H or halo) may be synthesized as shown generally in Scheme 1. A starting 3,6-dichloropyridazine (i) may be reacted with sodium iodide under microwave irradiation and heating in hydroiodic acid to provide an intermediate mono-iodo-chloropyridazine, which may be further reacted with copper(I) cyanide in acetonitrile and heating up to around 150-170° C. under microwave irradiation to provide intermediate (ii). Intermediate (ii) may be reacted with a tetrahydronapthyridine (iii) in a solvent such as N-methylpyrrolidone in the presence of a base (e.g., Hünig's base) and heating up to 100-120° C. to provide the product (iv).

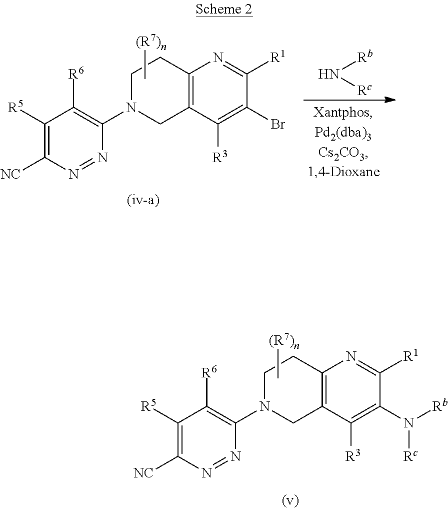

As shown in Scheme 2, intermediate (iv-a) may be coupled with an amine under Buchwald coupling conditions, generally known in the art, to provide products (v), wherein R^{b} and R^{c} are as defined herein. For example, the reaction may be conducted with a palladium catalyst such as Pd₂(dba)₃ in the presence of a base (e.g., Cs₂CO₃) and a ligand such as Xantphos (4,5-bis(diphenylphosphino)-9,9-dimethylxanthene) in a solvent such as dioxane with heating up to around 100° C. The synthetic route depicted in Scheme 2 may likewise be applied to the synthesis of compounds of formula (I) wherein R² is G² and G² is a heterocycle attached at a nitrogen atom (e.g., piperidin-1-yl).

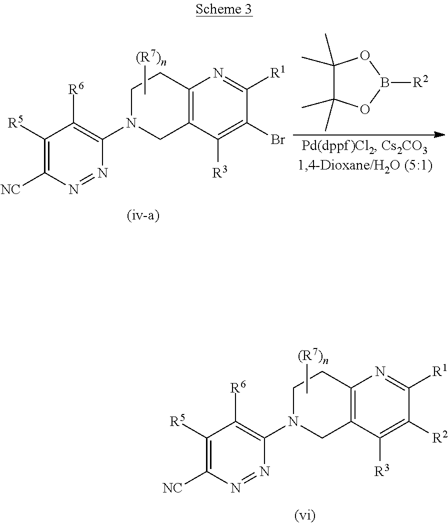

As shown in Scheme 3, intermediate (iv-a) may be coupled with a boronic acid or ester under Suzuki coupling conditions, generally known in the art, to provide compounds of formula (vi), wherein R² is alkyl or G² and G² is an optionally substituted aryl or heteroaryl ring system as defined herein. Coupling reactions may be conducted with a palladium catalyst such as Pd(dppf)Cl₂ and a base (e.g., K₂CO₃, Cs₂CO₃) in a solvent mixture of organic solvent and water such as DMF or dioxane and water with heating to about 70-90° C. The reaction may be facilitated with microwave irradiation.

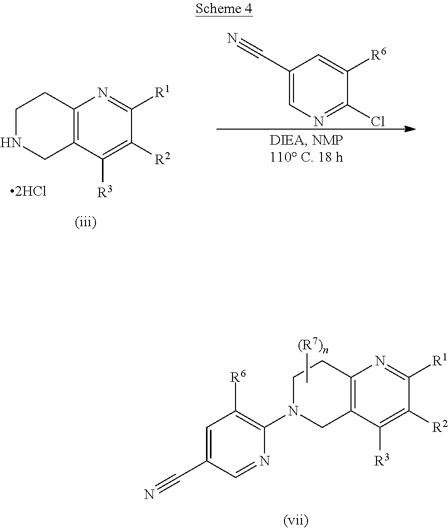

As shown in Scheme 4, intermediate (iii) may be converted to compounds of formula (vii) (e.g., R² is H or halo) using procedures analogous to those described for Scheme 1.

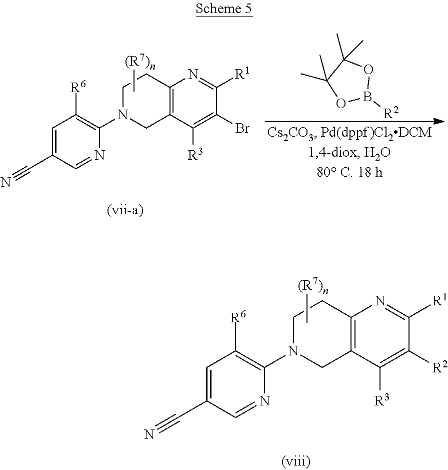

As shown in Scheme 5, compounds of formula (vii-a) may be converted to compounds of formula (viii) using procedures analogous to those described for Scheme 3, wherein R² is alkyl or G² and G² is an optionally substituted aryl or heteroaryl ring system as defined herein.

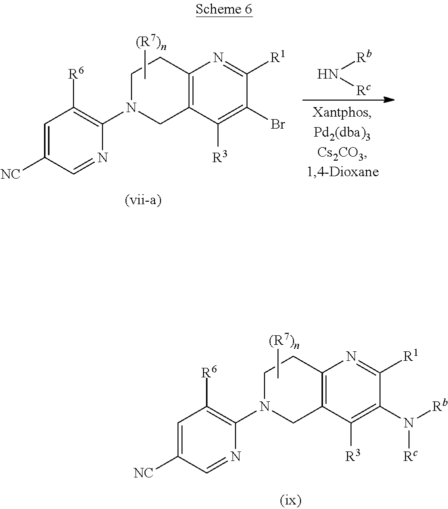

As shown in Scheme 6, compounds of formula (vii-a) may be converted to compounds of formula (ix) using procedures analogous to those described for Scheme 2. The synthetic route depicted in Scheme 6 may likewise be applied to the synthesis of compounds of formula (I) wherein R² is G² and G² is a heterocycle attached at a nitrogen atom (e.g., piperidin-1-yl).

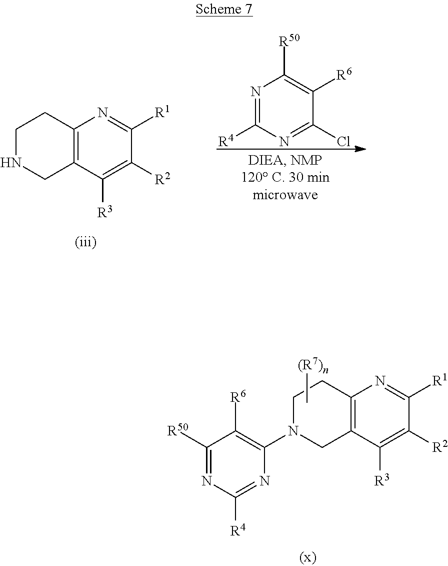

As shown in Scheme 7, intermediate (iii) may be converted to compounds of formula (x) (e.g., R² is H or halo) using procedures analogous to those described for Scheme 1.

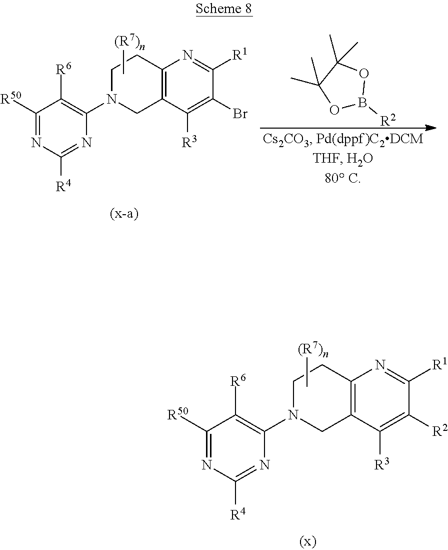

As shown in Scheme 8, compounds of formula (x-a) may be converted to compounds of formula (x), wherein R² is alkyl or G² and G² is an optionally substituted aryl or heteroaryl ring system as defined herein, using procedures analogous to those described for Scheme 3.

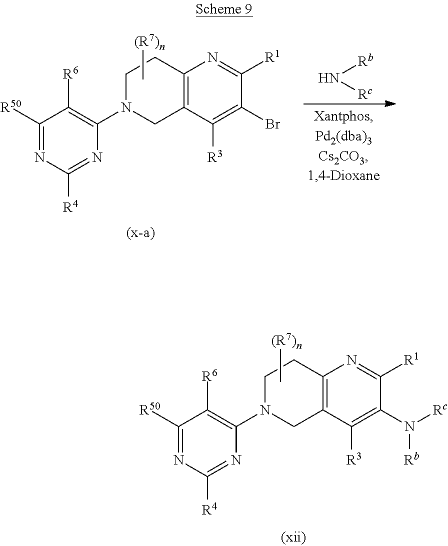

As shown in Scheme 9, compounds of formula (x-a) may be converted to compounds of formula (xii) using procedures analogous to those described for Scheme 2. The synthetic route depicted in Scheme 9 may likewise be applied to the synthesis of compounds of formula (I) wherein R² is G² and G² is a heterocycle attached at a nitrogen atom (e.g., piperidin-1-yl).

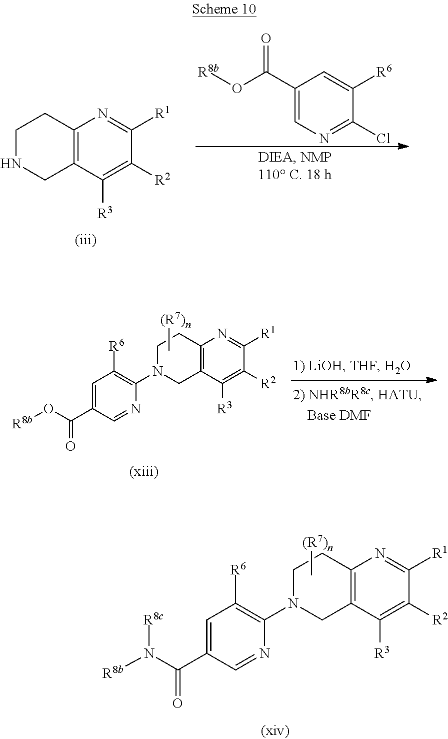

As shown in Scheme 10, compounds of formula (iii) may be converted to compounds of formula (xiii) (e.g., R² is H or halo) using procedures analogous to those described for Scheme 1. In turn, ester compounds of formula (xiii) may be converted to compounds of formula (xiv) by hydrolysis under standard conditions (e.g., LiGH, THF/water) to provide the carboxylic acid (not shown), which may be converted to amides (xiv) under standard coupling conditions (e.g., HATU, HNR^{8b}R^{8c}, Hünig's base, DMF).

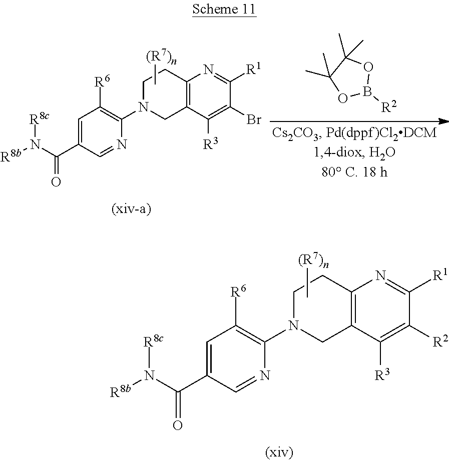

As shown in Scheme 11, compounds of formula (xiv-a) may be converted to compounds of formula (xiv), wherein R² is alkyl or G² and G² is an optionally substituted aryl or heteroaryl ring system as defined herein, using procedures analogous to those described for Scheme 3.

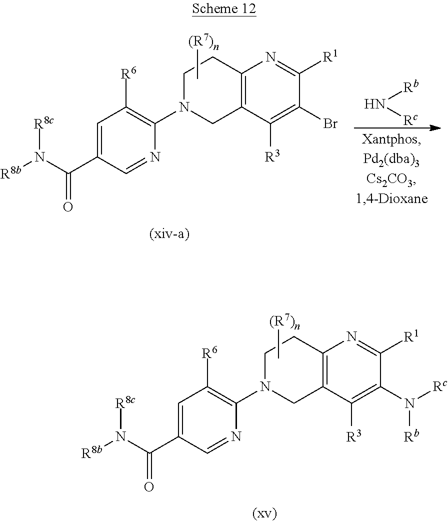

As shown in Scheme 12, compounds of formula (xiv-a) may be converted to compounds of formula (xv) using procedures analogous to those described for Scheme 2. The synthetic route depicted in Scheme 12 may likewise be applied to the synthesis of compounds of formula (I) wherein R² is G² and G² is a heterocycle attached at a nitrogen atom (e.g., piperidin-1-yl).

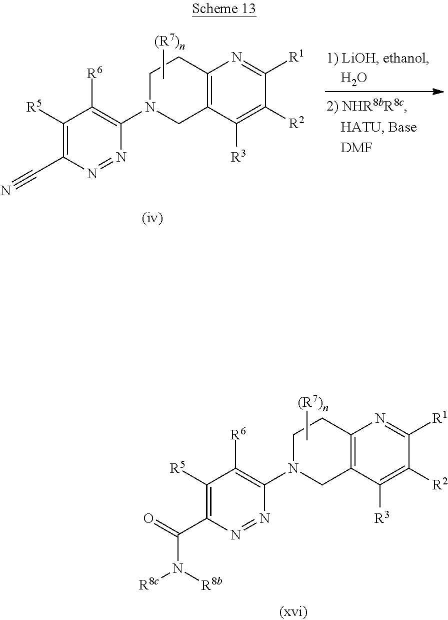

As shown in Scheme 13, nitrile compounds of formula (iv) may be converted to compounds of formula (xvi) by hydrolysis under standard conditions (e.g., LiGH, ethanol/water) to provide the carboxylic acid (not shown), which may be converted to amides (xvi) under standard coupling conditions (e.g., HATU, HNR^{8b}R^{8c}, Hünig's base, DMF).

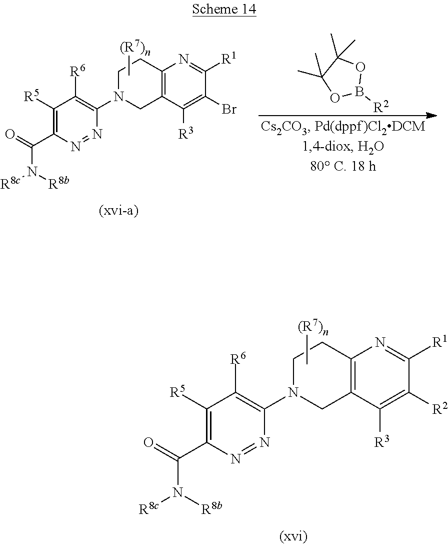

As shown in Scheme 14, compounds of formula (xvi-a) may be converted to compounds of formula (xvi), wherein R² is alkyl or G² and G² is an optionally substituted aryl or heteroaryl ring system as defined herein, using procedures analogous to those described for Scheme 3.

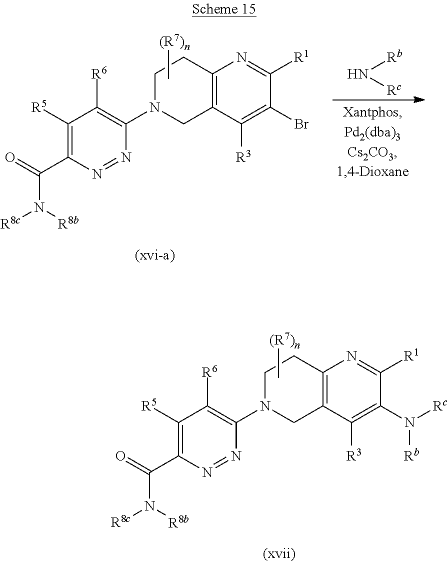

As shown in Scheme 15, compounds of formula (xvi-a) may be converted to compounds of formula (xvii) using procedures analogous to those described for Scheme 2. The synthetic route depicted in Scheme 15 may likewise be applied to the synthesis of compounds of formula (I) wherein R² is G² and G² is a heterocycle attached at a nitrogen atom (e.g., piperidin-1-yl).

The following materials are also available from commercial sources to prepare compounds of the invention according to the schemes and examples disclosed herein: METHYL 6-FLUORO-5-METHYLNICOTINATE (CAS #211122-38-2; Combi-Blocks, Inc.; catalog #QK-2095); 6—CHLORO-5-METHYLNICOTINONITRILE (CAS #66909-33-9; AstaTech, Inc.; catalog #22676); 3,6-DICHLORO-4,5-DIMETHYLPYRIDAZINE (CAS #34584-69-5; Combi-Blocks, Inc.; catalog #QB-2518); 4,6-DICHLORO-2-CYCLOPROPYL-5-METHYLPYRIMIDINE (CAS #21721-73-3; Enamine; catalog #EN300-92015); 4,6-DICHLORO-2,5-DIMETHYLPYRIMIDINE (CAS #1780-33-2; AstaTech, Inc.; catalog #60106).

Suitable boronic acids/esters, amines, and alcohols for coupling reactions described herein may be readily obtained from commercial sources or prepared by standard methods well know to those skilled in the art.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

c. Muscarinic Acetylcholine Receptor M₄ Activity

In some embodiments, the disclosed compounds potentiate the agonist response (e.g., acetylcholine) of mAChR M₄. In some embodiments, the disclosed compounds increase mAChR M₄ response to non-maximal concentrations of agonist in the presence of compound compared to the response to agonist in the absence of compound. The potentiation of mAChR M₄ activity can be demonstrated by methodology known in the art. For example, activation of mAChR M₄ activity can be determined by measurement of calcium flux in response to agonist, e.g. acetylcholine, in cells loaded with a Ca²⁺-sensitive fluorescent dye (e.g., Fluo-4) and co-expression of a chimeric or promiscuous G protein. In some embodiments, the calcium flux was measured as an increase in fluorescent static ratio. In some embodiments, positive allosteric modulator activity was analyzed as a concentration-dependent increase in the EC₂₀ acetylcholine response (i.e. the response of mAChR M₄ at a concentration of acetylcholine that yields 20% of the maximal response).

In some embodiments, the disclosed compounds activate mAChR M₄ response as an increase in calcium fluorescence in mAChR M₄-transfected CHO-K1 cells in the presence of the compound, compared to the response of equivalent CHO-K1 cells in the absence of the compound. In some embodiments, a disclosed compound activates the mAChR M₄ response with an EC₅₀ of less than about 10 μM, less than about 5 μM, less than about 1 μM, less than about 500 nM, of less than about 100 nM, or less than about 50 nM. In some embodiments, the mAChR M₄-transfected CHO-K1 cells are transfected with human mAChR M₄ In some embodiments, the mAChR M₄-transfected CHO-K1 cells are transfected with rat mAChR M₄.

The disclosed compounds may exhibit positive allosteric modulation of mAChR M₄ response to acetylcholine as an increase in response to non-maximal concentrations of acetylcholine in CHO-K1 cells transfected with a mAChR M₄ in the presence of the compound, compared to the response to acetylcholine in the absence of the compound. In some embodiments, the disclosed compounds exhibit positive allosteric modulation of the mAChR M₄ response to acetylcholine with an EC₅₀ of less than about 10 μM, less than about 5 μM, less than about 1 μM, less than about 500 nM, or less than about 100 nM. In some embodiments, the EC₅₀ for positive allosteric modulation is determined in CHO-K1 cells are transfected with a mAChR M₄. In some embodiments, the mAChR M₄ transfected human mAChR M₄. In some embodiments, the mAChR M₄ transfected rat mAChR M₄.

The disclosed compounds may activate mAChR M₄ response in mAChR M₄-transfected CHO-K1 cells with an EC₅₀ less than the EC₅₀ for one or more of mAChR M₁, M₂, M₃ or M₅-transfected CHO-K1 cells. That is, a disclosed compound can have selectivity for the mAChR M₄ receptor vis-a-vis one or more of the mAChR M₁, M₂, M₃ or M₅ receptors. For example, in some embodiments, a disclosed compound can activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₁. In some embodiments, a disclosed compound can activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₂. In some embodiments, a disclosed compound can activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₃. In some embodiments, a disclosed compound can activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₅. In some embodiments, a disclosed compound can activate mAChR M₄ response with an EC₅₀ of 5-fold less, about 10-fold less, about 20-fold less, about 30-fold less than that for the M₂-M₅ receptors, of about 50-fold less, about 100-fold less, about 200-fold less, about 300-fold less, about 400-fold less, or greater than about 500-fold less than that for the mAChR M₁, M₂, M₃, or M₅ receptors.

The disclosed compounds may activate mAChR M₄ response in M₄-transfected CHO-K1 cells with an EC₅₀ of less than about 10 μM and exhibits a selectivity for the M₄ receptor vis-a-vis one or more of the mAChR M₁, M₂, M₃, or M₅ receptors. For example, in some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, 10-fold less, 20-fold less, 30-fold less, 50-fold less, 100-fold less, 200-fold less, 300-fold less, 400-fold less, or greater than about 500-fold less than that for mAChR M₁. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₂. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₃. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₅. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with EC₅₀ of 5-fold less, about 10-fold less, about 20-fold less, about 30-fold less than that for the M₂-M₅ receptors, of about 50-fold less, about 100-fold less, about 200-fold less, about 300-fold less, about 400-fold less, M₂, M₃, or M₅ receptors, or greater than about 500-fold less than that for the mAChR M₁, M₂, M₃, or M₅ receptors.

In vivo efficacy for disclosed compounds can be measured in a number of preclinical rat behavioral models where known, clinically useful antipsychotics display similar positive responses. For example, disclosed compounds may reverse amphetamine-induced hyperlocomotion in male Sprague-Dawley rats at doses ranging from 1 to 100 mg/kg p.o.

### 3. Pharmaceutical Compositions and Formulations

The disclosed compounds may be incorporated into pharmaceutical compositions suitable for administration to a subject (such as a patient, which may be a human or non-human). The disclosed compounds may also be provided as formulations, such as spray-dried dispersion formulations.

The pharmaceutical compositions and formulations may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their pharmaceutically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp.587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

The pharmaceutical composition or formulation may exhibit positive allosteric modulation of mAChR M₄ with an EC₅₀ of less than about 10 μM, less than about 5 μM, less than about 1 μM, less than about 500 nM, or less than about 100 nM. The pharmaceutical composition or formulation may exhibit positive allosteric modulation of mAChR M₄ with an EC₅₀ of between about 10 μM and about 1 nM, about 1 μM and about 1 nM, about 100 nM and about 1 nM, or between about 10 nM and about 1 nM.

a. Spray-Dried Dispersion Formulations

The disclosed compounds may be formulated as a spray-dried dispersion (SDD). An SDD is a single-phase, amorphous molecular dispersion of a drug in a polymer matrix. It is a solid solution with the compound molecularly “dissolved” in a solid matrix. SDDs are obtained by dissolving drug and a polymer in an organic solvent and then spray-drying the solution. The use of spray drying for pharmaceutical applications can result in amorphous dispersions with increased solubility of Biopharmaceutics Classification System (BCS) class II (high permeability, low solubility) and class IV (low permeability, low solubility) drugs. Formulation and process conditions are selected so that the solvent quickly evaporates from the droplets, thus allowing insufficient time for phase separation or crystallization. SDDs have demonstrated long-term stability and manufacturability. For example, shelf lives of more than 2 years have been demonstrated with SDDs. Advantages of SDDs include, but are not limited to, enhanced oral bioavailability of poorly water-soluble compounds, delivery using traditional solid dosage forms (e.g., tablets and capsules), a reproducible, controllable and scalable manufacturing process and broad applicability to structurally diverse insoluble compounds with a wide range of physical properties.

Thus, in one embodiment, the disclosure may provide a spray-dried dispersion formulation comprising a compound of formula (I).

### 4. Methods of Use

The disclosed compounds, pharmaceutical compositions and formulations may be used in methods for treatment of disorders, such as neurological and/or psychiatric disorders, associated with muscarinic acetylcholine receptor dysfunction. The disclosed compounds and pharmaceutical compositions may also be used in methods for the potentiation of muscarinic acetylcholine receptor activity in a mammal, and in methods for enhancing cognition in a mammal. The methods further include cotherapeutic methods for improving treatment outcomes in the context of cognitive or behavioral therapy. In the methods of use described herein, additional therapeutic agent(s) may be administered simultaneously or sequentially with the disclosed compounds and compositions.

a. Treating Disorders

The disclosed compounds, pharmaceutical compositions and formulations may be used for treating disorders, or used in methods for treatment of disorders, such as neurological and/or psychiatric disorders, associated with muscarinic acetylcholine receptor dysfunction. The methods of treatment may comprise administering to a subject in need of such treatment a therapeutically effective amount of the compound of formula (I), or a pharmaceutical composition comprising a therapeutically effective amount of a compound of formula (I).

In some embodiments, the disclosure provides a method for enhancing cognition in a mammal comprising the step of administering to the mammal a therapeutically effective amount of the compound of formula (I), or a pharmaceutical composition comprising a therapeutically effective amount of a compound of formula (I).

The compounds and compositions disclosed herein may be useful for treating, preventing, ameliorating, controlling or reducing the risk of a variety of disorders associated with selective mAChR M₄ receptor activation. For example, a treatment can include selective mAChR M₄ receptor activation to an extent effective to affect cholinergic activity. A disorder can be associated with cholinergic activity, for example cholinergic hypofunction. Thus, provided is a method of treating or preventing a disorder in a subject comprising the step of administering to the subject at least one disclosed compound or at least one disclosed pharmaceutical composition, in an amount effective to treat the disorder in the subject.

Also provided is a method for the treatment of one or more disorders associated with mAChR M₄ receptor activity in a subject comprising the step of administering to the subject a therapeutically effective amount of the compound of formula (I), or a pharmaceutically acceptable salt thereof, or a pharmaceutical composition comprising a therapeutically effective amount of a compound of formula (I), or a pharmaceutically acceptable salt thereof.

In some embodiments, the disclosure provides a compound of formula (I), or a pharmaceutically acceptable salt thereof, for use in a method for the treatment of a disorder associated with the mAChR M₄ receptor. In some embodiments, the disclosure provides a pharmaceutical composition comprising a compound of formula (I), or a pharmaceutically acceptable salt thereof, for use in a method for the treatment of a disorder associated with the mAChR M₄ receptor.

In some embodiments, the disclosure provides a compound of formula (I), or a pharmaceutically acceptable salt thereof, for use in the manufacture of a medicament for the treatment of a disorder associated with the mAChR M₄ receptor.

In some embodiments, the disclosure provides a method for the treatment of a disorder associated with muscarinic acetylcholine receptor dysfunction in a mammal, comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In some embodiments, the disclosure provides a compound of formula (I), or a pharmaceutically acceptable salt thereof, for use in a method for the treatment of a disorder associated with muscarinic acetylcholine receptor dysfunction in a mammal.

In some embodiments, the disclosure provides a pharmaceutical composition comprising a compound of formula (I), or a pharmaceutically acceptable salt thereof, for use in a method for the treatment of a disorder associated with muscarinic acetylcholine receptor dysfunction in a mammal.

In some embodiments, the disclosure provides a compound of formula (I), or a pharmaceutically acceptable salt thereof, for use in the manufacture of a medicament for the treatment of a disorder associated with muscarinic acetylcholine receptor dysfunction in a mammal.

In some embodiments, the disclosed compounds and compositions have utility in treating a variety of neurological, psychiatric and cognitive disorders associated with the mAChR M₄ receptor, including one or more of the following conditions or diseases: schizophrenia, psychotic disorder NOS, brief psychotic disorder, schizophreniform disorder, schizoaffective disorder, delusional disorder, shared psychotic disorder, catastrophic schizophrenia, postpartum psychosis, psychotic depression, psychotic break, tardive psychosis, myxedematous psychosis, occupational psychosis, menstrual psychosis, secondary psychotic disorder, bipolar I disorder with psychotic features, and substance-induced psychotic disorder. In some embodiments, the psychotic disorder is a psychosis associated with an illness selected from major depressive disorder, affective disorder, bipolar disorder, electrolyte disorder, Alzheimer's disease, neurological disorder, hypoglycemia, AIDS, lupus, and post-traumatic stress disorder.

In some embodiments, the disclosure provides a compound of formula (I), or a pharmaceutically acceptable salt thereof, for use in a method for the treatment of a neurological, psychiatric, or cognitive disorder associated with the mAChR M₄ receptor, in particular, the disorders described herein. In some embodiments, the disclosure provides a pharmaceutical composition comprising a compound of formula (I), or a pharmaceutically acceptable salt thereof, for use in a method for the treatment of a neurological, psychiatric, or cognitive disorder associated with the mAChR M₄ receptor, in particular, the disorders described herein. In some embodiments, the disclosure provides a compound of formula (I), or a pharmaceutically acceptable salt thereof, for use in the manufacture of a medicament for the treatment of a neurological, psychiatric, or cognitive disorder associated with the mAChR M₄ receptor, in particular, the disorders described herein.

In some embodiments, the disorder is a neurological disorder selected from brain tumor, dementia with Lewy bodies, multiple sclerosis, sarcoidosis, Lyme disease, syphilis, Alzheimer's disease, Parkinson's disease, and anti-NMDA receptor encephalitis.

In some embodiments, the disorder is a psychotic disorder selected from schizophrenia, brief psychotic disorder, schizophreniform disorder, schizoaffective disorder, delusional disorder, and shared psychotic disorder. In some embodiments, the schizophrenia is selected from catastrophic schizophrenia, catatonic schizophrenia, paranoid schizophrenia, residual schizophrenia, disorganized schizophrenia, and undifferentiated schizophrenia. In some embodiments, the disorder is selected from schizoid personality disorder, schizotypal personality disorder, and paranoid personality disorder. In some embodiments, the psychotic disorder is due to a general medical condition and is substance-induced or drug-induced (phencyclidine, ketamine and other dissociative anesthetics, amphetamine and other psychostimulants, and cocaine).

In some embodiments, the present disclosure provides a method for treating a cognitive disorder, comprising administering to a patient in need thereof an effective amount of a compound or a composition of the present disclosure. In some embodiments, cognitive disorders include dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse), delirium, amnestic disorder, substance-induced persisting delirium, dementia due to HIV disease, dementia due to Huntington's disease, dementia due to Parkinson's disease, Parkinsonian-ALS demential complex, dementia of the Alzheimer's type, age-related cognitive decline, and mild cognitive impairment.

The text revision of the fourth edition of the Diagnostic and Statistical Manual of Mental Disorders (DSM-IV-TR) (2000, American Psychiatric Association, Washington DC) provides a diagnostic tool that includes cognitive disorders including dementia, delirium, amnestic disorders and age-related cognitive decline. The fifth edition of the Diagnostic and Statistical Manual of Mental Disorders (DSM-5) (2013, American Psychiatric Association, Washington DC) provides a diagnostic tool for neurocognitive disorders (NCDs) that include delirium, followed by the syndromes of major NCD, mild NCD, and their etiological subtypes. The major or mild NCD subtypes include NCD due to Alzheimer's disease, vascular NCD, NCD with Lewy bodies, NCD due to Parkinson's disease, frontotemporal NCD, NCD due to traumatic brain injury, NCD due to HIV infection, substance/medication-induced NCD, NCD due to Huntington's disease, NCD due to prion disease, NCD due to another medical condition, NCD due to multiple etiologies, and unspecified NCD. The NCD category in DSM-5 encompasses the group of disorders in which the primary clinical deficit is in cognitive function, and that are acquired rather than developmental. As used herein, the term “cognitive disorders” includes treatment of those cognitive disorders and neurocognitive disorders as described in DSM-IV-TR or DSM-5. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In some embodiments, the present disclosure provides a method for treating schizophrenia or psychosis, comprising administering to a patient in need thereof an effective amount of a compound or composition of the present disclosure. Particular schizophrenia or psychosis pathologies are paranoid, disorganized, catatonic or undifferentiated schizophrenia and substance-induced psychotic disorder. DSM-IV-TR provides a diagnostic tool that includes paranoid, disorganized, catatonic, undifferentiated or residual schizophrenia, and substance-induced psychotic disorder. DSM-5 eliminated the subtypes of schizophrenia, and instead includes a dimensional approach to rating severity for the core symptoms of schizophrenia, to capture the heterogeneity in symptom type and severity expressed across individuals with psychotic disorders. As used herein, the term “schizophrenia or psychosis” includes treatment of those mental disorders as described in DSM-IV-TR or DSM-5. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

In some embodiments, the present disclosure provides a method for treating pain, comprising administering to a patient in need thereof an effective amount of a compound or composition of the present disclosure. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

The compounds and compositions may be further useful in a method for the prevention, treatment, control, amelioration, or reduction of risk of the diseases, disorders and conditions noted herein. The compounds and compositions may be further useful in a method for the prevention, treatment, control, amelioration, or reduction of risk of the aforementioned diseases, disorders and conditions, in combination with other agents.

In the treatment of conditions which require activation of mAChR M₄, an appropriate dosage level may be about 0.01 to 500 mg per kg patient body weight per day, which can be administered in single or multiple doses. The dosage level may be about 0.1 to about 250 mg/kg per day, or about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, or 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in some embodiments, the disclosure relates to a method for activating mAChR M₄ receptor activity in at least one cell, comprising the step of contacting the at least one cell with at least one disclosed compound or at least one product of a disclosed method in an amount effective to activate mAChR M₄ in the at least one cell. In some embodiments, the cell is mammalian, for example, human. In some embodiments, the cell has been isolated from a subject prior to the contacting step. In some embodiments, contacting is via administration to a subject.

In some embodiments, the invention relates to a method for activating mAChR M₄ activity in a subject, comprising the step of administering to the subject at least one disclosed compound or at least one product of a disclosed method in a dosage and amount effective to activating mAChR M₄ activity in the subject. In some embodiments, the subject is mammalian, for example, human. In some embodiments, the mammal has been diagnosed with a need for mAChR M₄ agonism prior to the administering step. In some embodiments, the mammal has been diagnosed with a need for mAChR M₄ activation prior to the administering step. In some embodiments, the method further comprises the step of identifying a subject in need of mAChR M₄ agonism.

In some embodiments, the invention relates to a method for the treatment of a disorder associated with selective mAChR M₄ activation, for example, a disorder associated with cholinergic activity, in a mammal comprising the step of administering to the mammal at least one disclosed compound or at least one product of a disclosed method in a dosage and amount effective to treat the disorder in the mammal. In some embodiments, the mammal is a human. In some embodiments, the mammal has been diagnosed with a need for treatment for the disorder prior to the administering step. In some embodiments, the method further comprises the step of identifying a subject in need of treatment for the disorder.

In some embodiments, the disorder can be selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

In some embodiments, the disorder is Alzheimer's disease.

b. Potentiation of Muscarinic Acetylcholine Receptor Activity

In some embodiments, the disclosure relates to a method for potentiation of muscarinic acetylcholine receptor activity in a mammal comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In some embodiments, the disclosure provides a compound of formula (I), or a pharmaceutically acceptable salt thereof, for use in a method for the potentiation of muscarinic acetylcholine receptor activity in a mammal. In some embodiments, the disclosure provides a pharmaceutical composition comprising a compound of formula (I), or a pharmaceutically acceptable salt thereof, for use in a method for the potentiation of muscarinic acetylcholine receptor activity in a mammal.

In some embodiments, the disclosure provides a compound of formula (I), or a pharmaceutically acceptable salt thereof, for use in the manufacture of a medicament for the potentiation of muscarinic acetylcholine receptor activity in a mammal.

In some embodiments, potentiation of muscarinic acetylcholine receptor activity increases muscarinic acetylcholine receptor activity. In some embodiments, potentiation of muscarinic acetylcholine receptor activity is partial agonism of the muscarinic acetylcholine receptor. In some embodiments, potentiation of muscarinic acetylcholine receptor activity is positive allosteric modulation of the muscarinic acetylcholine receptor.

In some embodiments, the compound administered exhibits potentiation of mAChR M₄ with an EC₅₀ of less than about 10 μM, less than about 5 μM, less than about 1 μM, less than about 500 nM, or less than about 100 nM. In some embodiments, the compound administered exhibits potentiation of mAChR M₄ with an EC₅₀ of between about 10 μM and about 1 nM, about 1 μM and about 1 nM, about 100 nM and about 1 nM, or about 10 nM and about 1 nM.

In some embodiments, the mammal is a human. In some embodiments, the mammal has been diagnosed with a need for potentiation of muscarinic acetylcholine receptor activity prior to the administering step. In some embodiments, the method further comprises the step of identifying a mammal in need of potentiating muscarinic acetylcholine receptor activity. In some embodiments, the potentiation of muscarinic acetylcholine receptor activity treats a disorder associated with muscarinic acetylcholine receptor activity in the mammal. In some embodiments, the muscarinic acetylcholine receptor is mAChR M₄.

In some embodiments, potentiation of muscarinic acetylcholine receptor activity in a mammal is associated with the treatment of a neurological and/or psychiatric disorder associated with a muscarinic receptor dysfunction, such as a neurological or psychiatric disorder disclosed herein. In some embodiments, the muscarinic receptor is mAChR M₄.

In some embodiments, the disclosure provides to a method for potentiation of muscarinic acetylcholine receptor activity in a cell, comprising the step of contacting the cell with an effective amount of at least one disclosed compound or a pharmaceutically acceptable salt thereof. In some embodiments, the cell is mammalian (e.g., human). In some embodiments, the cell has been isolated from a mammal prior to the contacting step. In some embodiments, contacting is via administration to a mammal.

c. Enhancing Cognition

In some embodiments, the invention relates to a method for enhancing cognition in a mammal comprising the step of administering to the mammal an effective amount of least one disclosed compound; or a pharmaceutically acceptable salt, hydrate, solvate, or polymorph thereof.

In some embodiments, the disclosure provides a compound of formula (I), or a pharmaceutically acceptable salt thereof, for use in a method for the enhancment of cognition in a mammal. In some embodiments, the disclosure provides a pharmaceutical composition comprising a compound of formula (I), or a pharmaceutically acceptable salt thereof, for use in a method for the enhancment of cognition in a mammal.

In some embodiments, the disclosure provides a compound of formula (I), or a pharmaceutically acceptable salt thereof, for use in the manufacture of a medicament for the enhancment of cognition in a mammal.

In some embodiments, the mammal is a human. In some embodiments, the mammal has been diagnosed with a need for cognition enhancement prior to the administering step. In some embodiments, the method further comprises the step of identifying a mammal in need of cognition enhancement. In some embodiments, the need for cognition enhancement is associated with a muscarinic receptor dysfunction. In some embodiments, the muscarinic receptor is mAChR M₄.

In some embodiments, the cognition enhancement is a statistically significant increase in Novel Object Recognition. In some embodiments, the cognition enhancement is a statistically significant increase in performance of the Wisconsin Card Sorting Test.

d. Cotherapeutic methods

The present invention is further directed to administration of a selective mAChR M₄ activator for improving treatment outcomes in the context of cognitive or behavioral therapy. That is, in some embodiments, the invention relates to a cotherapeutic method comprising a step of administering to a mammal an effective amount and dosage of at least one disclosed compound, or a pharmaceutically acceptable salt thereof.

In some embodiments, the disclosure provides a compound of formula (I), or a pharmaceutically acceptable salt thereof, for use in a cotherapeutic method with cognitive or behavioral therapy in a mammal. In some embodiments, the disclosure provides a pharmaceutical composition comprising a compound of formula (I), or a pharmaceutically acceptable salt thereof, for use in a cotherapeutic method with cognitive or behavioral therapy in a mammal.

In some embodiments, the disclosure provides a compound of formula (I), or a pharmaceutically acceptable salt thereof, for use in the manufacture of a medicament for a cotherapeutic method with cognitive or behavioral therapy in a mammal.

In some embodiments, administration improves treatment outcomes in the context of cognitive or behavioral therapy. Administration in connection with cognitive or behavioral therapy can be continuous or intermittent. Administration need not be simultaneous with therapy and can be before, during, and/or after therapy. For example, cognitive or behavioral therapy can be provided within 1, 2, 3, 4, 5, 6, 7 days before or after administration of the compound. As a further example, cognitive or behavioral therapy can be provided within 1, 2, 3, or 4 weeks before or after administration of the compound. As a still further example, cognitive or behavioral therapy can be provided before or after administration within a period of time of 1, 2, 3, 4, 5, 6, 7, 8, 9, or 10 half-lives of the administered compound.

It is understood that the disclosed cotherapeutic methods can be used in connection with the disclosed compounds, compositions, kits, and uses.

e. Combination Therapies

In the methods of use described herein, additional therapeutic agent(s) may be administered simultaneously or sequentially with the disclosed compounds and compositions. Sequential administration includes administration before or after the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula (I). The above combinations include combinations of a compound of the present invention not only with one other active compound, but also with two or more other active compounds.

The disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefor, contemporaneously or sequentially with a disclosed compound. When a disclosed compound is used contemporaneously with one or more other drugs, a pharmaceutical composition in unit dosage form containing such drugs and the disclosed compound may be used. However, the combination therapy can also be administered on overlapping schedules. It is also envisioned that the combination of one or more active ingredients and a disclosed compound can be more efficacious than either as a single agent. Thus, when used in combination with one or more other active ingredients, the disclosed compounds and the other active ingredients can be used in lower doses than when each is used singly.

The pharmaceutical compositions and methods of the present invention can further comprise other therapeutically active compounds as noted herein which are usually applied in the treatment of the above mentioned pathological conditions.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the disclosed compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In some embodiments, the compound can be employed in combination with anti-Alzheimer's agents, beta-secretase inhibitors, cholinergic agents, gamma-secretase inhibitors, HMG-CoA reductase inhibitors, M₁ allosteric agonists, M₁ positive allosteric modulators, NSAIDs including ibuprofen, vitamin E, and anti-amyloid antibodies. In another embodiment, the subject compound can be employed in combination with sedatives, hypnotics, anxiolytics, antipsychotics (typical and atypical), antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject compound can be administered in conjunction with the use of physical methods such as with light therapy or electrical stimulation.

In some embodiments, the compound can be employed in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexol) hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist can be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In some embodiments, the compound can be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound can be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound can be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In some embodiments, the compound can be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, a-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRY) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In some embodiments, the compounds can be coadministered with orthosteric muscarinic agonists, muscarinic potentiators, or cholinesterase inhibitors. In some embodiments, the compounds can be coadministered with GlyT1 inhibitors and the like such as, but not limited to: risperidone, clozapine, haloperidol, fluoxetine, prazepam, xanomeline, lithium, phenobarbitol, and salts thereof and combinations thereof.

f. Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire.TM.). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

### 5. Kits

In one aspect, the disclosure provides kits comprising at least one disclosed compound or a pharmaceutically acceptable salt thereof, and one or more of:
(a) at least one agent known to increase mAChR M₄ activity;
(b) at least one agent known to decrease mAChR M₄ activity;
(c) at least one agent known to treat a disorder associated with cholinergic activity;
(d) instructions for treating a disorder associated with cholinergic activity;
(e) instructions for treating a disorder associated with M₄ receptor activity; or
(f) instructions for administering the compound in connection with cognitive or behavioral therapy.

In some embodiments, the at least one disclosed compound and the at least one agent are co-formulated. In some embodiments, the at least one disclosed compound and the at least one agent are co-packaged. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

The disclosed kits can be employed in connection with disclosed methods of use.

The kits may further comprise information, instructions, or both that use of the kit may provide treatment for medical conditions in mammals (particularly humans). The information and instructions may be in the form of words, pictures, or both, and the like. In addition or in the alternative, the kit may include the compound, a composition, or both; and information, instructions, or both; regarding methods of application of compound, or of composition, for example with the benefit of treating or preventing medical conditions in mammals (e.g., humans).

The compounds and processes of the invention will be better understood by reference to the following examples, which are intended as an illustration of and not a limitation upon the scope of the invention.

### 6. EXAMPLES

All NMR spectra were recorded on a 400 MHz AMX Bruker NMR spectrometer. ¹H chemical shifts are reported in 6 values in ppm downfield with the deuterated solvent as the internal standard. Data are reported as follows: chemical shift, multiplicity (s=singlet, bs=broad singlet, d=doublet, t=triplet, q=quartet, dd=doublet of doublets, m=multiplet, ABq=AB quartet), coupling constant, integration. Reversed-phase LCMS analysis was performed using an Agilent 1200 system comprised of a binary pump with degasser, high-performance autosampler, thermostatted column compartment, C18 column, diode-array detector (DAD) and an Agilent 6150 MSD with the following parameters. The gradient conditions were 5% to 95% acetonitrile with the aqueous phase 0.1% TFA in water over 1.4 minutes, hold at 95% acetonitrile for 0.1 min, 0.5 mL/min, 55° C. (“90 sec method”). Samples were separated on a Waters Acquity UPLC BEH C18 column (1.7 μm, 1.0×50 mm) at 0.5 mL/min, with column and solvent temperatures maintained at 55° C. The DAD was set to scan from 190 to 300 nm, and the signals used were 220 nm and 254 nm (both with a band width of 4 nm). The MS detector was configured with an electrospray ionization source, and the low-resolution mass spectra were acquired by scanning from 140 to 700 AMU with a step size of 0.2 AMU at 0.13 cycles/second, and peak width of 0.008 minutes. The drying gas flow was set to 13 liters per minute at 300° C. and the nebulizer pressure was set to 30 psi. The capillary needle voltage was set at 3000 V, and the fragmentor voltage was set at 100V. Data acquisition was performed with Agilent Chemstation and Analytical Studio Reviewer software.

Abbreviations used in the examples and reaction schemes that follow include the following:
ACN or MeCN=acetonitrile;
DCM=dichloromethane
DIEA or DIPEA=diisopropylethyl amine
DMF=N,N-dimethylformamide
DMSO=dimethyl sulfoxide
EtOAc=ethyl acetate
EtOH=ethanol
h or hr=hour(s)
HATU=2-(7-aza-1H-benzotriazole-1-yl)-1,1,3,3-tetramethyluronium hexafluorophosphate
IPA=isopropyl alcohol
MeCN=acetonitrile
MeOH=methanol
min=minute(s)
NMP=N-methyl-2-pyrrolidone
Pd₂(dba)₃=tris(dibenzylideneacetone)dipalladium(0)
Pd(dppf)Cl₂=[1,1′-Bis(diphenylphosphino)ferrocene]palladium(II) dichloride
RP=reverse phase
rt or r.t.=room temperature
R-r=retention time (in minutes)
TFA is trifluoroacetic acid
THE=tetrahydrofuran

Xantphos is 4,5-bis(diphenylphosphino)-9,9-dimethylxanthene

### Example 1

### 6-(3-Bromo-7,8-dihydro-1,6-naphthyridin-6(5H)-yl)-4,5-dimethylpyridazine-3-carbonitrile (B)

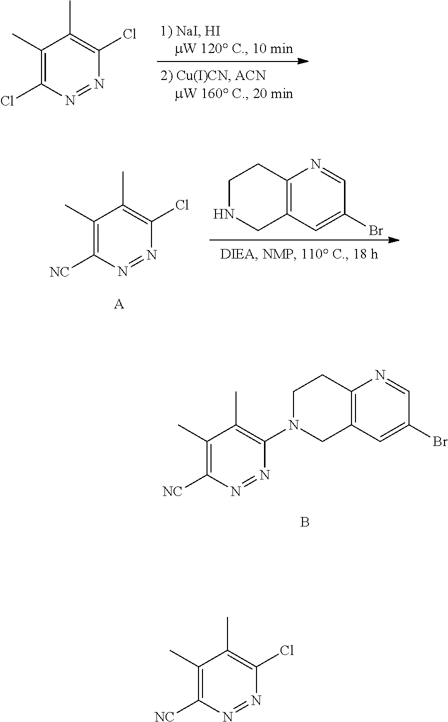

6-Chloro-4,5-dimethyl-pyridazine-3-carbonitrile (A). Divided equally among 5×20 mL microwave vials were added 3,6-dichloro-4,5-dimethylpyridazine (5 g, 28.2 mmol) and sodium iodide (10.6 g, 70.6 mmol) dissolved in hydroiodic acid (50 mL). The reaction vessel was sealed and subjected to microwave irradiation at 120° C. for 10 min. The reaction was quenched by slowly adding (dropwise) into a flask containing a mixture of saturated aqueous NaHCO₃(˜150 mL), saturated aqueous Na₂S₂O₃ (˜50 mL), and DCM (˜50 mL). The mixture was stirred for 30 minutes then extracted with DCM (3×). The solvent was removed and the crude product (a pale yellow powder) was carried on without further purification.

Divided equally among 6×20 mL microwave vials was added a mixture of 3-chloro-6-iodo-4,5-dimethyl-pyridazine (7.5 g, 27.8 mmol) and copper(I) cyanide (4.99 g, 55.7 mmol) in MeCN (66 mL) was heated to 160° C. for 20 min under microwave irradiation. DCM was added and the mixture was filtered through Celite®. The filtrate was concentrated in vacuo onto Celite® and the crude product was purified using normal phase chromatography (0-50% EtOAc/hexanes) to afford 6-chloro-4,5-dimethyl-pyridazine-3-carbonitrile (2.78 g, 16.6 mmol, 59% yield) as a pale yellow solid. ¹H NMR (400 MHz, CDCl₃) δ 2.58 (s, 3H), 2.47 (s, 3H). LCMS (90 sec method): RT=0.541 min, m/z=168.2 [M+H], >98% @215 nm and 254 nm.

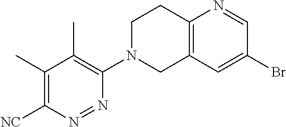 [SMILES: Cc1c(C#N)nnc(N2CCc3ncc(Br)cc3C2)c1C]

6-(3-Bromo-7,8-dihydro-1,6-naphthyridin-6(5H)-yl)-4,5-dimethylpyridazine-3-carbonitrile (B). To a solution of 6-chloro-4,5-dimethyl-pyridazine-3-carbonitrile (200 mg, 1.19 mmol) in NMP (5.8 mL) was added N,N-diisopropylethylamine (0.62 mL, 3.58 mmol) and 7-bromo-2,5-diazatetralin (279.7 mg, 1.31 mmol) and NMP (5.8 mL). The mixture was stirred at 110° C. for 8 h before cooling to room temperature and diluted with water (˜40 mL) and a precipitate formed. The precipitate was filtered off to give 6-(3-bromo-7,8-dihydro-5H-1,6-naphthyridin-6-yl)-4,5-dimethyl-pyridazine-3-carbonitrile (253 mg, 0.73 mmol, 62% yield). ¹H NMR (400 MHz, CDCl₃) δ 8.54 (d, J=2.3 Hz, 1H), 7.70 (bs, 1H), 4.71 (s, 2H), 3.64 (t, J=6.0 Hz, 2H), 3.22 (t, J=6.0 Hz, 2H), 2.48 (s, 3H), 2.33 (s, 2H). LCMS (90 sec method): RT=0.866; m/z=344.0 [M+H]*; >90% @215 nm & 254 nm.

### Example 2

### 6-(3-((2-Fluorophenyl)amino)-7,8-dihydro-1,6-naphthyridin-6(5H)-yl)-4,5-dimethylpyridazine-3-carbonitrile (C)

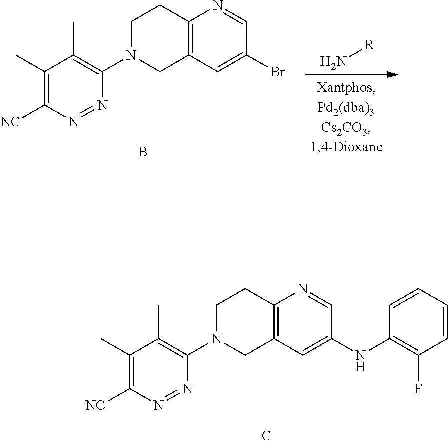

6-(3-((2-Fluorophenyl)amino)-7,8-dihydro-1,6-naphthyridin-6(5H)-yl)-4,5-dimethylpyridazine-3-carbonitrile (C). To a microwave vial under inert atmosphere was added tris(dibenzylideneacetone)dipalladium(0) (2.8 mg), 4,5-bis(diphenylphosphino)-9,9-dimethylxanthene (2.6 mg), 2-fluoroaniline (5.1 mg, 0.05 mmol), 6-(3-bromo-7,8-dihydro-5H-1,6-naphthyridin-6-yl)-4,5-dimethyl-pyridazine-3-carbonitrile (20 mg, 0.06 mmol), and cesium carbonate (0.01 mL, 0.09 mmol) in 1,4-dioxane (1.0 mL). The resulting suspension was heated to 100° C. for 18 hr, and LCMS confirmed complete loss of starting material. The reaction was cooled to r.t. and filtered through a plug of Celite®, washed with EtOAc/DCM (1:1), and the solvents were concentrated. The crude residue was dissolved in DMSO (1.5 mL) and purified using the RP-HPLC (10-45% ACN/0.1% aqueous TFA). The fractions containing product were neutralized with sat. NaHCO₃ and extracted with 3:1 chloroform/IPA. The organic layers were concentrated to afford the title compound (4.2 mg, 0.01 mmol, 20% yield) as a pale yellow solid. ¹H NMR (400 MHz, CDCl₃) δ 8.45 (s, 1H), 7.32 (s, 1H), 7.29-7.27 (m, 1H), 7.17-7.04 (m, 3H), 4.71 (s, 2H), 3.63 (t, J=5.9 Hz, 2H), 3.32 (t, J=5.9 Hz, 2H), 2.48 (s, 3H), 2.32 (s, 3H). LCMS (90 sec method): RT=0.739; m/z=375.2 [M+H]*; >98% @215 nm & 254 nm.

### Example 3

### 4,5-Dimethyl-6-(3-thiazol-5-yl-7,8-dihydro-5H-1,6-naphthyridin-6-yl)pyridazine-3-carbonitrile (D)

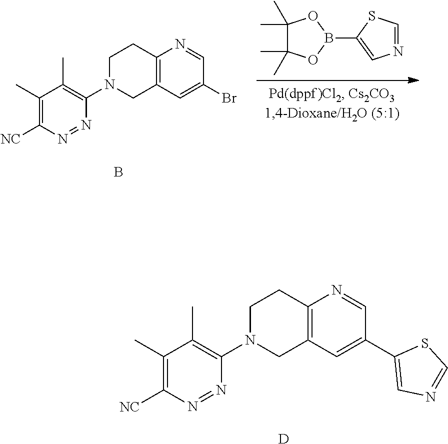

To a microwave vial was added a mixture of thiazol-5-ylboronic acid pinacol ester (12.3 mg, 0.06 mmol), cesium carbonate (56.8 mg, 0.17 mmol), [1,1′-bis(diphenylphosphino)ferrocene]dichloropalladium(II) (4.3 mg, 0.01 mmol), and 6-(3-bromo-7,8-dihydro-5H-1,6-naphthyridin-6-yl)-4,5-dimethyl-pyridazine-3-carbonitrile (20 mg, 0.06 mmol) were added to a vessel followed by 1,4-dioxane (0.53 mL) and water (0.05 mL, degassed). The vial was sealed, and the reaction was heated to 80° C. for 18 h. The reaction mixture was filtered through a pad of Celite®, washed with EtOAc/DCM then concentrated. The crude residue was dissolved in DMSO (1.5 mL) and purified using the reverse phase HPLC (10-45% ACN/0.1% aqueous TFA). The fractions containing product were neutralized with sat. NaHCO₃ and extracted with 3:1 chloroform/IPA. The solvents were concentrated to give 4,5-dimethyl-6-(3-thiazol-5-yl-7,8-dihydro-5H-1,6-naphthyridin-6-yl)pyridazine-3-carbonitrile as a white solid. ¹H NMR (400 MHz, CDCl₃) δ 8.86 (s, 1H), 8.71 (d, J=2.1 Hz, 1H), 8.15 (s, 1H), 7.76 (bs, 1H), 4.80 (s, 2H), 3.68 (t, J=5.9 Hz, 2H), 3.33 (t, J=5.9 Hz, 2H), 2.49 (s, 3H), 2.35 (s, 3H). LCMS (90 sec method): RT=0.691; m/z=349.2 [M+H]+; >98% @215 nm & 254 nm.

### Example 4

### 6-[3-(1-Isobutylpyrazol-4-yl)-7,8-dihydro-5H-1,6-naphthyridin-6-yl]-5-methyl-pyridine-3-carbonitrile

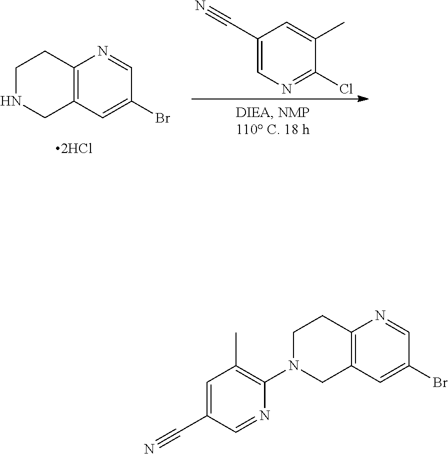

6-(3-Bromo-7,8-dihydro-5H-1,6-naphthyridin-6-yl)-5-methyl-pyridine-3-carbonitrile. To a solution of 6-chloro-5-methylnicotinonitrile (450.0 mg, 3.0 mmol) in NMP (14 mL) was added DIEA (2.6 mL, 15 mmol) and 3-bromo-5,6,7,8-tetrahydro-1,6-naphthyridine dihydrochloride (1.0 g, 3.5 mmol). The reaction mixture was stirred at 110° C. for 18 h before cooling to room temperature. The mixture was diluted with sat. NaHCO₃ (200 mL) and extracted with EtOAc (3×). The collected organic layers were dried (MgSO₄), filtered and concentrated. The crude mixture was purified using normal phase chromatography (0-50% EtOAc/hexanes). ¹H NMR (400 MHz, d-DMSO) δ 8.54 (d, J=2.2 Hz, 1H), 8.50 (d, J=2.3 Hz, 1H), 7.98 (d, J=2.2 Hz, 1H), 7.92 (dd, J=2.2, 0.8 Hz, 1H), 4.60 (s, 2H), 3.68 (dd, J=11.8, 5.9 Hz, 2H), 3.02 (dd, J=11.6, 5.6 Hz, 2H), 2.35 (s, 3H). ES-MS [M+H]⁺=329.2.

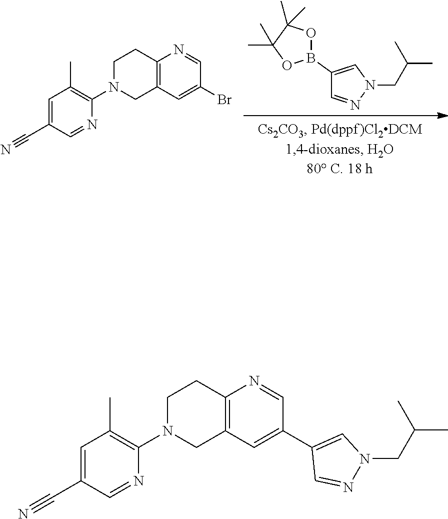 [SMILES: Cc1cc(C#N)cnc1N1CCc2ncc(Br)cc2C1 ; CC(C)Cn1cc(B2OC(C)(C)C(C)(C)O2)cn1 ; Cc1cc(C#N)cnc1N1CCc2ncc(-c3cnn(CC(C)C)c3)cc2C1]

6-[3-(1-Isobutylpyrazol-4-yl)-7,8-dihydro-5H-1,6-naphthyridin-6-yl]-5-methyl-pyridine-3-carbonitrile. To a microwave vial was added a mixture of 1-isobutylpyrazole-4-boronic acid pinacol ester (7.6 mg, 0.03 mmol), cesium carbonate (30 mg, 0.09 mmol), Pd(dppf)Cl₂ DCM (2.2 mg, 0.003 mmol), and 6-(3-bromo-7,8-dihydro-5H-1,6-naphthyridin-6-yl)-5-methyl-pyridine-3-carbonitrile (10 mg, 0.03 mmol). To the mixture was added 1,4-dioxane (0.9 mL) and water (0.09 mL). After the vial was evacuated and purged with N₂ (3×), the reaction was heated to 80° C. for 18 h. The reaction mixture was filtered through a pad of Celite® and washed with EtOAc/DCM, then concentrated. The crude residue was dissolved in DMSO (1.5 mL) and purified by RP-HPLC (5-35% MeCN/0.1% TFA aqueous solution). The fractions containing product were basified with sat. NaHCO₃ and extracted with 3:1 chloroform/IPA. The solvents were concentrated to give 6-[3-(1-isobutylpyrazol-4-yl)-7,8-dihydro-5H-1,6-naphthyridin-6-yl]-5-methyl-pyridine-3-carbonitrile (7.1 mg, 63% yield) as a yellow solid. ¹H NMR (400 MHz, d-DMSO) δ 8.70 (d, J=2.1 Hz, 1H), 8.58 (d, J=2.2 Hz, 1H), 8.29 (s, 1H), 8.03 (s, 1H), 7.97 (d, J=1.5 Hz, 1H), 7.88 (d, J=2.0 Hz, 1H), 4.67 (s, 2H), 4.01 (d, J=7.3 Hz, 2H), 3.76 (dd, J=11.7, 5.9 Hz, 2H), 3.08 (dd, J=11.4, 5.5 Hz, 2H), 2.42 (s, 3H), 2.23-2.17 (m, 1H), 0.97 (s, 3H), 0.89 (s, 3H). ES-MS [M+H]⁺=373.4.

### Example 5

### 6-[3-[(2-Fluoro-6-methyl-3-pyridyl)amino]-7,8-dihydro-5H-1,6-naphthyridin-6-yl]-5-methyl-pyridine-3-carbonitrile

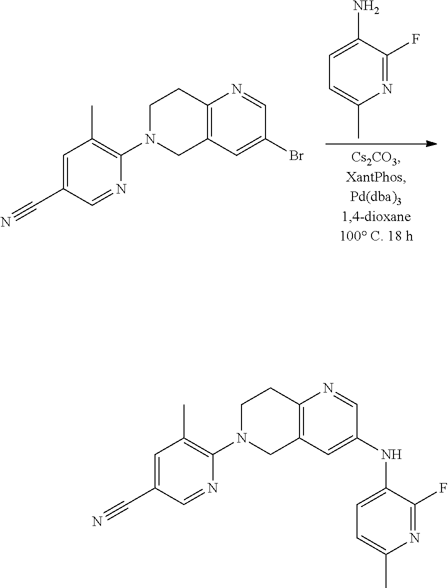

To a microwave vial under inert atmosphere was added Pd₂(dba)₃ (2.8 mg, 0.003 mmol), Xantphos (2.6 mg, 0.005 mmol), 6-(3-bromo-7,8-dihydro-5H-1,6-naphthyridin-6-yl)-5-methyl-pyridine-3-carbonitrile (10 mg, 0.03 mmol), 3-amino-2-fluoro-6-methylpyridine (5.8 mg, 0.05 mmol), and cesium carbonate (0.01 mL, 0.09 mmol) in 1,4-dioxane (1.0 mL). The resulting suspension was heated to 100° C. for 18 hr, after which time LCMS confirmed loss of starting material. The reaction was cooled to r.t. and filtered through a plug of Celite with EtOAc, and solvents were removed. The crude material was purified by RP-HPLC (15-45% MeCN in 0.1% TFA aqueous solution over 10 min). The fractions containing product were basified with sat. NaHCO₃ and extracted with 3:1 chloroform/IPA. The solvents were concentrated to give 6-[3-[(2-fluoro-6-methyl-3-pyridyl)amino]-7,8-dihydro-5H-1,6-naphthyridin-6-yl]-5-methyl-pyridine-3-carbonitrile (2 mg, 0.005 mmol, 18% yield) as a brown solid. ¹H NMR (400 MHz, CDCl₃) δ 8.49 (s, 1H), 8.31 (d, J=1.9 Hz, 1H), 7.57 (dd, J=2.1, 0.6 Hz, 1H), 7.53-7.50 (m, 1H), 7.13 (s, 1H), 6.92 (d, J=7.7 Hz, 1H), 4.49 (s, 2H), 3.60 (dd, J=11.7, 6.0 Hz, 2H), 3.22 (dd, J=11.1, 5.5 Hz, 2H), 2.43 (s, 3H), 2.29 (s, 3H). ES-MS [M+H]⁺=375.4.

### Example 6

### 3-Bromo-6-(6-chloro-2,5-dimethyl-pyrimidin-4-yl)-7,8-dihydro-5H-1,6-naphthyridine

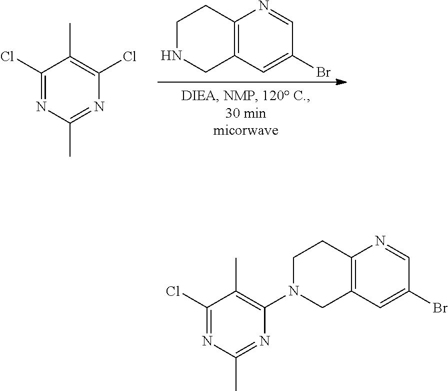

A mixture of 4,6-dichloro-2,5-dimethylpyrimidine (500 mg, 2.82 mmol, 1.0 eq.), 3-bromo-5,6,7,8-tetrahydro-1,6-naphthyridine dihydrochloride (808 mg, 2.82 mmol, 1.0 eq.) and N,N-diisopropylethylamine (1.48 mL, 8.47 mmol, 3.0 eq.) in NMP (7.06 mL, 0.4 M) was subjected under microwave irradiation at 120° C. for 30 min. After irradiation, the reaction mixture was diluted with water and extracted with EtOAc (3×). The combined extracts were washed with water, brine, dried over Na₂SO₄, filtered and concentration. Purification using column chromatography on silica gel (0-50% EtOAc/hexanes) provided the title compound as a crystalline solid (915 mg, 92%). ¹H NMR (400 MHz, CDCl₃) δ 8.54 (d, J=2.2 Hz, 1H), 7.68 (d, J=1.9 Hz, 1H), 4.61 (s, 2H), 3.70 (dd, J=5.9, 5.9 Hz, 2H), 3.18 (dd, J=5.9, 5.9 Hz, 2H), 2.58 (s, 3H), 2.32 (s, 3H); ES-MS [M+H]⁺=353.2/355.2.

### Example 7

### 6-(6-Chloro-2,5-dimethyl-pyrimidin-4-yl)-N-(2-fluorophenyl)-7,8-dihydro-5H-1,6-naphthyridin-3-amine

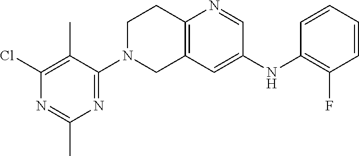 [SMILES: Cc1nc(Cl)c(C)c(N2CCc3ncc(Nc4ccccc4F)cc3C2)n1]

3-Bromo-6-(6-chloro-2,5-dimethyl-pyrimidin-4-yl)-7,8-dihydro-5H-1,6-naphthyridine (15 mg, 0.04 mmol, 1.0 eq.), tris(dibenzylideneacetone)dipalladium(0) (3.9 mg, 0.004 mmol, 0.1 eq.), 4,5-bis(diphenylphosphino)-9,9-dimethylxanthene (2.45 mg, 0.004 mmol, 0.1 eq.), 2-fluoroaniline (6.1 μL, 0.064 mmol, 1.5 eq.) and cesium carbonate (42 mg, 0.13 mmol, 3 eq.) in 1,4-dioxane (1.0 mL) were charged into a reaction vial under inert atmosphere. The resulting suspension was stirred at 100° C. After 90 min, the reaction mixture was cooled to r.t. and filtered through a plug of Celite, which was rinsed thoroughly with EtOAc/DCM (1:1). The filtrate was concentrated, and the crude material was purified using RP-HPLC (15-55% MeCN in 0.1% TFA aqueous solution). After work-up and extraction, the title compound was obtained as an off white solid (8.3 mg, 51%). ¹H NMR (400 MHz, CDCl₃) δ 8.56 (s, 1H), 7.36-7.32 (m, 2H), 7.22-7.11 (m, 4H), 4.60 (s, 2H), 3.39 (dd, J=5.8, 5.8 Hz, 2H), 3.36 (dd, J=5.8, 5.8 Hz, 2H), 2.55 (s, 3H), 2.32 (s, 3H); ES-MS [M+H]⁺=384.2.

### Example 8

### 6-(6-Chloro-2,5-dimethyl-pyrimidin-4-yl)-3-(1-methylpyrazol-4-yl)-7,8-dihydro-5H-1,6-naphthyridine

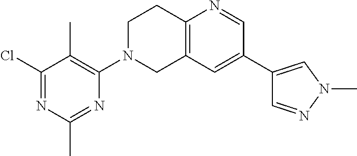 [SMILES: Cc1nc(Cl)c(C)c(N2CCc3ncc(-c4cnn(C)c4)cc3C2)n1]

3-Bromo-6-(6-chloro-2,5-dimethyl-pyrimidin-4-yl)-7,8-dihydro-5H-1,6-naphthyridine (10 mg, 0.028 mmol, 1.0 eq.), 1-methylpyrazole-4-boronic acid pinacol ester (7.1 mg, 0.034 mmol, 1.2 eq.), cesium carbonate (27.6 mg, 0.084 mmol, 3.0 eq.), and [1,1′-bis(diphenylphosphino)ferrocene]dichloropalladium(II) (3.1 mg, 0.004 mmol, 0.15 eq.), were charged into a reaction vial under inert atmosphere. A mixture of degassed THE and water (5:1, v/v, 1.0 mL) was added. The resulting solution was stirred at 80° C. After 90 min, the reaction mixture was filtered through a pad of Celite® which was rinsed thoroughly with EtOAc/DCM. The filtrate was concentrated, and the crude material was purified using RP-HPLC (10-50% MeCN in 0.1% TFA aqueous solution). After work-up and extraction, the title compound was obtained as a pale yellow solid (3.3 mg, 33%). ¹H NMR (400 MHz, CDCl₃) δ 8.54 (s, 1H), 7.83 (s, 1H), 7.74 (s, 1H), 7.70 (s, 1H), 4.63 (s, 2H), 3.91 (s, 3H), 3.64 (dd, J=5.9, 5.9 Hz, 2H), 3.41 (dd, J=5.9, 5.9 Hz, 2H), 2.47 (s, 3H), 2.25 (s, 3H); ES-MS [M+H]⁺=355.2.

### Example 9

### Methyl 6-(7,8-dihydro-1,6-naphthyridin-6(5H)-yl)-5-methylnicotinate

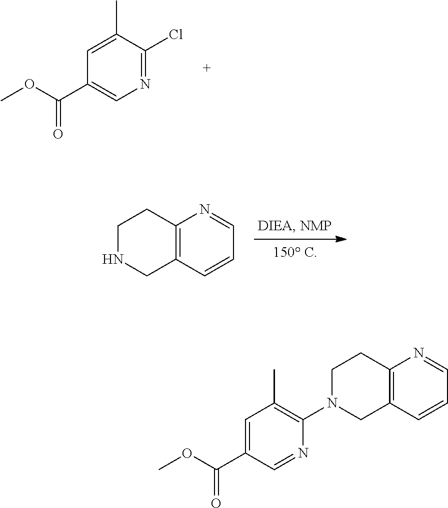 [SMILES: COC(=O)c1cnc(Cl)c(C)c1 ; c1cnc2c(c1)CNCC2 ; COC(=O)c1cnc(N2CCc3ncccc3C2)c(C)c1]

2,5-Diazatetralin (50 mg, 0.37 mmol) and methyl 6-chloro-5-methyl-pyridine-3-carboxylate (75 mg, 0.41 mmol) were dissolved in NMP (1.5 mL) and N,N-diisopropylethylamine (130 μL, 0.75 mmol) was added to the stirring solution. The vial was sealed and heated to 110° C. overnight (16 h). The reaction was cooled to r.t. and filtered prior to RP-HPLC (20-70% MeCN in 0.05% NH₄OH aqueous solution). The fractions containing pure compound were combined and concentrated under reduced pressure to yield the title compound as a yellow oil. (74 mg, 70% yield)¹H-NMR (400 MHz, CDCl₃) δ 8.76 (d, J=2.1 Hz, 1H), 8.45 (d, J=3.4 Hz, 1H), 8.00-7.97 (m, 1H), 7.47 (d, J=7.3 Hz, 1H), 7.13 (dd, J=7.7, 4.8 Hz, 1H), 4.57 (s, 2H), 3.90 (s, 3H), 3.61 (t, J=5.9, 2H), 3.20 (t, J=5.9, 2H), 2.38 (s, 3H). ES-MS [M+H]⁺=284.2.

### Example 10

### Lithium 6-(4-(1H-pyrazol-1-yl)benzyl)quinoline-8-carboxylate

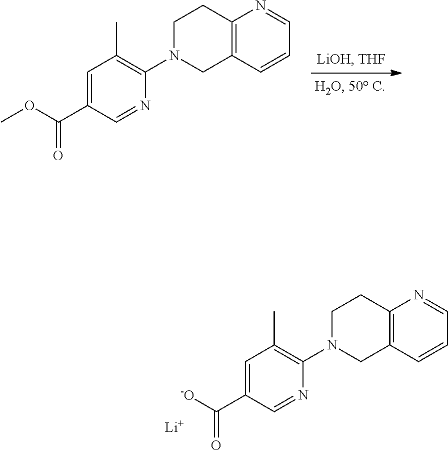

Methyl 6-(4-(1H-pyrazol-1-yl)benzyl)quinoline-8-carboxylate (74 mg, 0.26 mmol) was dissolved in 1,4-dioxane (1.5 mL) and a solution of LiGH (13 mg, 0.52 mmol) in H₂O (0.5 mL) was added dropwise. The resulting solution was heated to 50° C. and stirred for 2 h, after which time solvents were concentrated under reduced pressure to yield the title compound as a tan solid which was used without further purification. ES-MS [M+H]⁺=270.4.

### Example 11

### N-[(2-chlorothiazol-5-yl)methyl]-6-(7,8-dihydro-5H-1,6-naphthyridin-6-yl)-5-methyl-pyridine-3-carboxamide

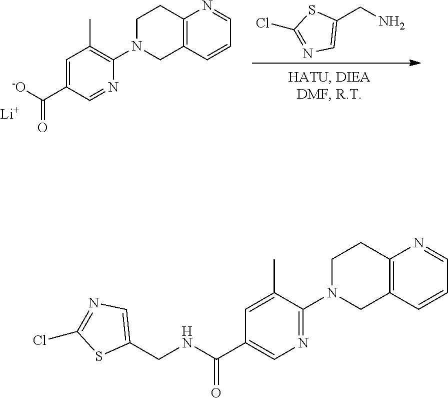

Lithium 6-(7,8-dihydro-5H-1,6-naphthyridin-6-yl)-5-methyl-pyridine-3-carboxylate (7.mg, 0.025 mmol) and HATU (14.51 mg, 0.038 mmol) were dissolved in DMF (0.5 mL) and allowed to stir for 15 min. 2-Chloro-5-(aminomethyl)thiazole (11.3 mg, 0.075 mmol) and N,N-diisopropylethylamine (13 μL, 0.075 mmol) were then added to the mixture. The reaction was stirred for 2 hours. The reaction was passed through a syringe filter and purified via RP-HPLC (25-80% MeCN/0.05% NH₄OH aqueous solution). The fractions containing pure compound were concentrated to yield the title compound (2.2 mg, 22% yield) as a yellow oil. ¹H-NMR (400 MHz, CDCl₃) δ 8.50 (d, J=2.3 Hz, 1H), 8.44 (d, J=3.4 Hz, 1H), 7.87-7.85 (m, 1H), 7.46 (d, J=9.4 Hz, 1H), 7.45 (s, 1H) 7.13 (dd, J=7.7, 4.8 Hz, 1H), 6.53 (t, J=5.3 Hz, 1H) 4.71 (d, J=5.3 Hz, 2H), 4.53 (s, 2H), 3.59 (t, J=5.9 Hz, 2H) 3.18 (t, J=5.9 Hz, 2H) 2.38 (s, 3H). ES-MS [M+H]⁺=400.0.

### Example 12

### 6-(3-Bromo-7,8-dihydro-1,6-naphthyridin-6(5H)-yl)-4,5-dimethylpyridazine-3-carboxylic acid

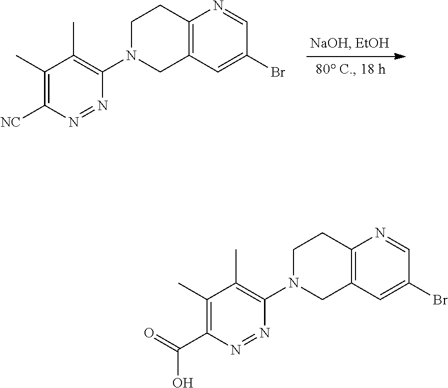 [SMILES: Cc1c(C#N)nnc(N2CCc3ncc(Br)cc3C2)c1C ; Cc1c(C(=O)O)nnc(N2CCc3ncc(Br)cc3C2)c1C]

To a solution of 6-(3-bromo-7,8-dihydro-5H-1,6-naphthyridin-6-yl)-4,5-dimethyl-pyridazine-3-carbonitrile (50 mg, 0.15 mmol) in ethanol (0.7 mL) was added a solution (1 Min water) of sodium hydroxide (436 μL, 0.44 mmol). The reaction mixture was heated to 80° C. Upon completion, the mixture was diluted with water and acidified with 1 MHCl to a pH ˜5 and extracted with chloroform/IPA (4:1) (2×). The combined organic extracts were dried over sodium sulfate, filtered, and concentrated to give 6-(3-bromo-7,8-dihydro-5H-1,6-naphthyridin-6-yl)-4,5-dimethyl-pyridazine-3-carboxylic acid (45 mg, 85% yield). ES-MS [M+H]⁺=363.0.

### Example 13

### 6-(3-Bromo-7,8-dihydro-1,6-naphthyridin-6(5H)-yl)-4,5-dimethyl-N-(pyridin-4-ylmethyl)pyridazine-3-carboxamide

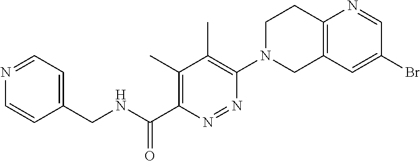 [SMILES: Cc1c(C(=O)NCc2ccncc2)nnc(N2CCc3ncc(Br)cc3C2)c1C]

To a solution of 6-(3-bromo-7,8-dihydro-5H-1,6-naphthyridin-6-yl)-4,5-dimethyl-pyridazine-3-carboxylic acid (45 mg, 0.12 mmol) in DMF (2 mL) was added HATU (94 mg, 0.25 mmol) and N,N-diisopropylethylamine (43 μL, 0.25 mmol). The mixture stirred for 10 minutes, then 4-(aminomethyl)pyridine (25 μL, 0.25 mmol) was added to the reaction mixture. The mixture was stirred at room temperature. Upon completion, the reaction was diluted with saturated sodium bicarbonate (˜35 mL) and extracted with EtOAc (3×15 mL). The organic phases were dried over sodium sulfate, filtered, and concentrated. The crude product was purified using normal phase chromatography (0-6% MeOH/DCM) to afford 6-(3-bromo-7,8-dihydro-5H-1,6-naphthyridin-6-yl)-4,5-dimethyl-N-(4-pyridylmethyl) pyridazine-3-carboxamide (38 mg, 69% yield). ES-MS [M+H]⁺=453.3/455.2.

### Example 14

### 4,5-Dimethyl-6-(3-(2-methylpyridin-4-yl)-7,8-dihydro-1,6-naphthyridin-6(5H)-yl)-N-(pyridin-4-ylmethyl)pyridazine-3-carboxamide

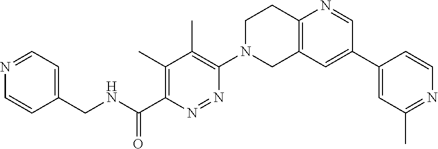 [SMILES: Cc1cc(-c2cnc3c(c2)CN(c2nnc(C(=O)NCc4ccncc4)c(C)c2C)CC3)ccn1]

To a microwave vial was added a mixture of picoline-4-boronic acid (9 mg, 0.06 mmol), cesium carbonate (69 mg, 0.21 mmol), [1,1′-bis(diphenylphosphino)ferrocene]dichloropalladium(II) (3 mg, 0.004 mmol), and 6-(3-bromo-7,8-dihydro-5H-1,6-naphthyridin-6-yl)-4,5-dimethyl-N-(4-pyridylmethyl)pyridazine-3-carboxamide (19 mg, 0.04 mmol) followed by 1,4-dioxane (200 μL) and water (50 μL). The vial was sealed, and the reaction was heated to 80° C. for 18 h. The reaction mixture was filtered through a plug of Celite® washing with DCM/EtOAc (1:1). The filtrate was concentrated, and the crude product was dissolve in DMSO (2 mL) and purified using a reverse-phase HPLC (20-55% ACN/0.05% aqueous NH₄OH). The fractions containing product were concentrated to give 4,5-dimethyl-6-[3-(2-methyl-4-pyridyl)-7,8-dihydro-5H-1,6-naphthyridin-6-yl]-N-(4-pyridylmethyl)pyridazine-3-carboxamide (5.2 mg, 0.011 mmol, 26% yield) as a tan solid. ES-MS [M+H]⁺=466.4.

### Example 15

### 6-(3-((2-Fluorophenyl)amino)-7,8-dihydro-1,6-naphthyridin-6(5H)-yl)-4,5-dimethyl-N-(pyridin-4-ylmethyl)pyridazine-3-carboxamide

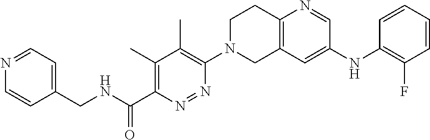 [SMILES: Cc1c(C(=O)NCc2ccncc2)nnc(N2CCc3ncc(Nc4ccccc4F)cc3C2)c1C]

To a microwave vial under inert atmosphere were added tris(dibenzylideneacetone)dipalladium(0) (4 mg, 0.004 mmol), 4,5-bis(diphenylphosphino)-9,9-dimethylxanthene (4 mg, 0.01 mmol), 2-fluoroaniline (6 μL, 0.06 mmol), 6-(3-bromo-7,8-dihydro-5H-1,6-naphthyridin-6-yl)-4,5-dimethyl-N-(4-pyridylmethyl)pyridazine-3-carboxamide (19 mg, 0.04 mmol), and cesium carbonate (42 mg, 0.13 mmol) in 1,4-dioxane (900 μL). The resulting suspension was heated to 110° C. for 1 h. The reaction was cooled to room temperature and filtered through a plug of Celite®, washed with EtOAc/DCM (1:1), and solvents were concentrated. The crude product was dissolved in DMSO (2 mL) and purified using the reverse-phase chromatography HPLC (30-60% ACN/0.05% aqueous NH₄OH). The fractions containing product were concentrated to give title compound as a tan solid ES-MS [M+H]⁺=484.4.

### Example 16

### 6-(3-(1,3-Dimethyl-1H-pyrazol-4-yl)-7,8-dihydro-1,6-naphthyridin-6(5H)-yl)-5-methyl-N-(thiazol-5-ylmethyl)nicotinamide

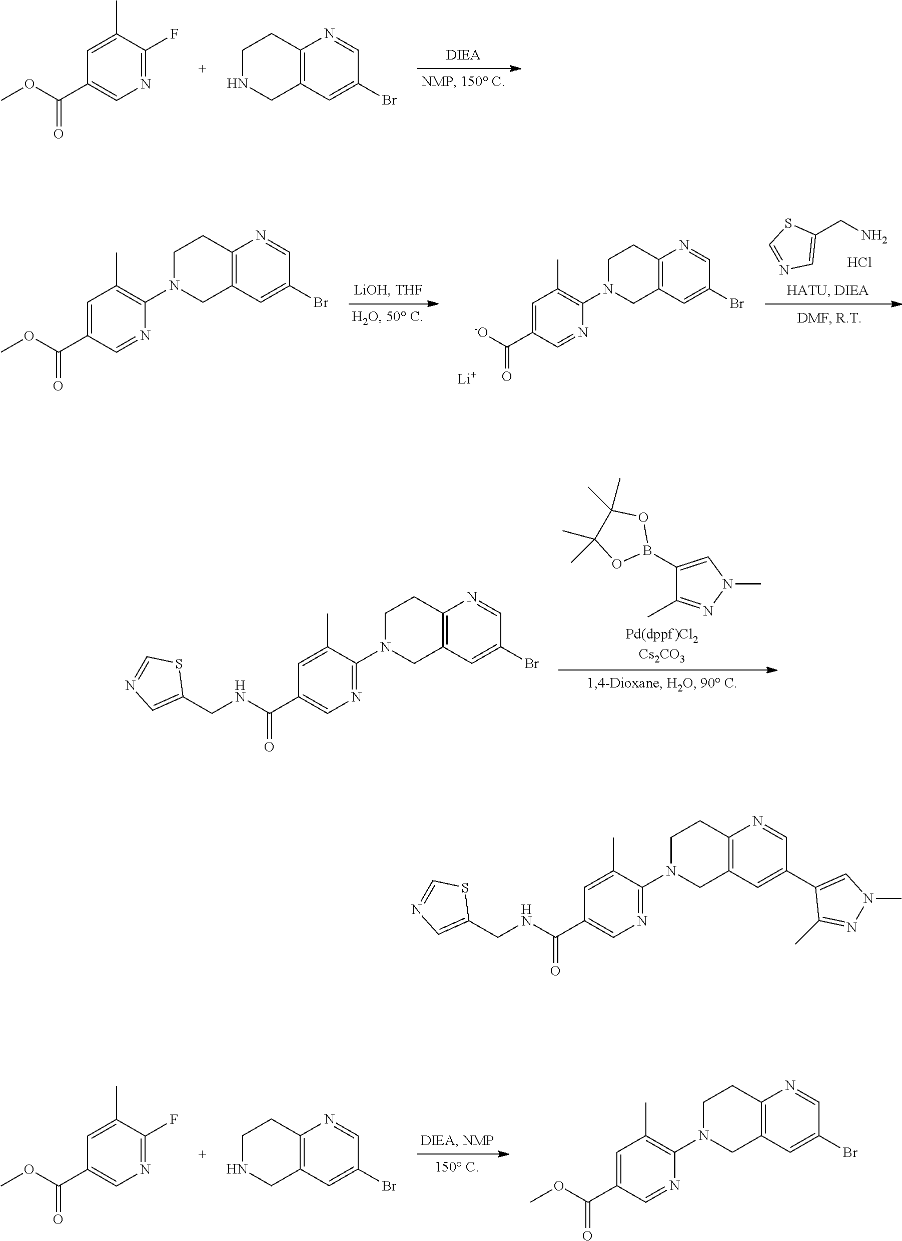

Methyl 6-(7,8-dihydro-1,6-naphthyridin-6(5H)-yl)-5-methylnicotinate. 3-Bromo-5,6,7,8-tetrahydro-1,6-naphthyridine (500 mg, 1.75 mmol) and methyl 6-fluoro-5-methylnicotinate (391 mg, 1.92 mmol) were dissolved in NMP (11.5 mL) and N,N-diisopropylethylamine (609 μL, 3.50 mmol) was added to the stirring solution. The vial was sealed and heated to 110° C. overnight (16 h). The reaction was cooled to r.t. and filtered prior to RP-HPLC (20-70% MeCN in 0.05% NH₄OH aqueous solution). The fractions containing pure compound were combined and concentrated under reduced pressure to yield the title compound as a yellow oil. (444 mg, 70% yield)¹H-NMR (400 MHz, CDCl₃) δ 8.76 (d, J=2.2 Hz, 1H), 8.51 (s, 1H), 8.02 (d, J=1.4, 1H), 7.68 (s, 1H), 4.60 (s, 1H), 3.90 (s, 3H), 3.62 (dd, J=5.8, 5.8, 2H), 3.17 (dd, J=5.4, 5.4, 2H), 2.38 (s, 3H). ES-MS [M+H]⁺=364.0.

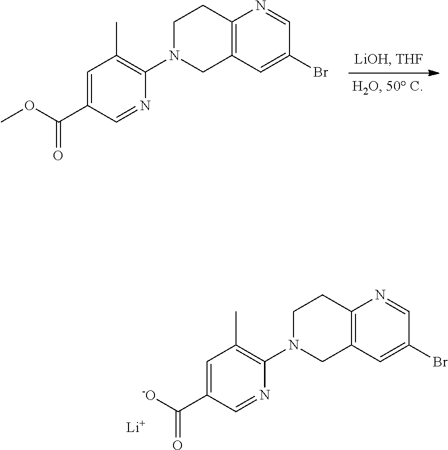

Lithium 6-(3-bromo-7,8-dihydro-1,6-naphthyridin-6(5H)-yl)-5-methylnicotinate. Methyl 6-(3-bromo-7,8-dihydro-5H-1,6-naphthyridin-6-yl)-5-methyl-pyridine-3-carboxylate (444 mg, 1.22 mmol) was dissolved in THE (6 mL) and a solution of LiGH (91.8 mg, 3.68 mmol) in H₂O (1.2 mL) was added dropwise. The resulting solution was heated to 50° C. and stirred for 2 h, after which time solvents were concentrated under reduced pressure to yield the title compound as a tan solid which was used without further purification. ES-MS [M+H]⁺=350.3.

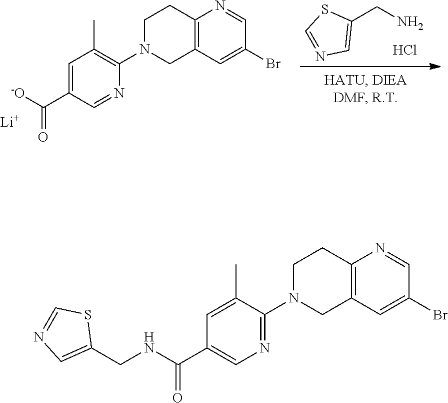

6-(3-Bromo-7,8-dihydro-1,6-naphthyridin-6(5H)-yl)-5-methyl-N-(thiazol-5-ylmethyl)nicotinamide. Lithium 6-(3-bromo-7,8-dihydro-1,6-naphthyridin-6(5H)-yl)-5-methylnicotinate (217 mg, 0.61 mmol) and HATU (350 mg, 0.92 mmol) were dissolved in DMF (3.0 mL). After 15 min, thiazol-5-ylmethanamine hydrochloride (185 mg, 1.23 mmol) and N,N-diisopropylethylamine (320 μL, 1.84 mmol) were then added to the mixture. After the reaction was complete via LCMS, the mixture was filtered, concentrated, and purified via RP-HPLC (25-80% MeCN in 0.05% aqueous NH₄OH). The fractions containing pure compound were concentrated to yield the title compound (206 mg, 75% yield) as a yellow powder. ¹H-NMR (400 MHz, CDCl₃) δ 8.73 (s, 1H), 8.68 (s, 1H), 8.51 (d, J=2.1 Hz, 1H), 8.06 (s, 1H), 7.85 (s, 1H), 7.69 (d, J=1.7 Hz, 1H), 4.85 (d, J=6.0 Hz, 2H), 4.66 (s, 2H), 3.67 (dd, J=5.8, 5.8 Hz, 2H) 3.48 (s, 1H) 3.17 (dd, J=5.8, 5.7 Hz, 2H) 2.41 (s, 3H). ES-MS [M+H]⁺=446.2.

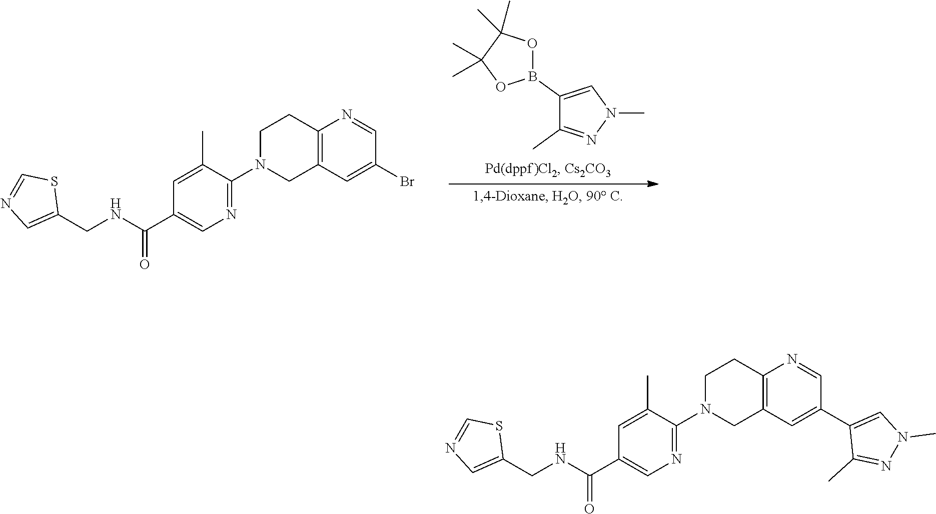

6-(3-(1,3-Dimethyl-1H-pyrazol-4-yl)-7,8-dihydro-1,6-naphthyridin-6(5H)-yl)-5-methyl-N-(thiazol-5-ylmethyl)nicotinamide. 6-(3-Bromo-7,8-dihydro-1,6-naphthyridin-6(5H)-yl)-5-methyl-N-(thiazol-5-ylmethyl)nicotinamide (10.0 mg, 0.023 mmol), cesium carbonate (22.0 mg, 0.068 mmol), Pd(ddpf)Cl₂ (1.65 mg, 0.002 mmol), and 1,3-dimethyl-4-(4,4,5,5-tetramethyl-1,3,2-dioxaborolan-2-yl)-1H-pyrazole (6.0 mg, 0.027 mmol) were combined into a small microwave vial. The vial was capped, sealed, and placed under an inert atmosphere. 1,4-Dioxane/water (5:1 v/v, 1 mL) was added. The mixture was evacuated and purged with nitrogen (3×) and heated to 90° C. After 1h, the heat was removed, and the crude reaction was concentrated. The residue was taken up in MeOH and passed through a syringe filter. The crude filtrate was purified via RP-HPLC (30-80% MeCN/0.05% aqueous NH₄OH) to yield the title compound (5 mg, 46% yield) as an oil. ES-MS [M+H]⁺=460.2.

### Example 17

### 6-(3-Bromo-7,8-dihydro-1,6-naphthyridin-6(5H)-yl)-5-methylpyridazine-3-carbonitrile

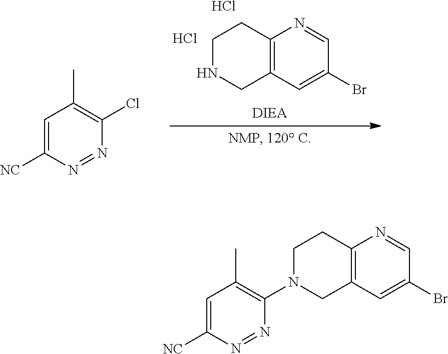

### Prepared in a similar manner as intermediate B to afford 184 mg of title compound (86% yield). ES-MS [M+1]⁺: 330.2/332.0

### Example 18

### 6-(3-(1-(Difluoromethyl)-1H-pyrazol-4-yl)-7,8-dihydro-1,6-naphthyridin-6(5H)-yl)-4,5-dimethylpyridazine-3-carbonitrile

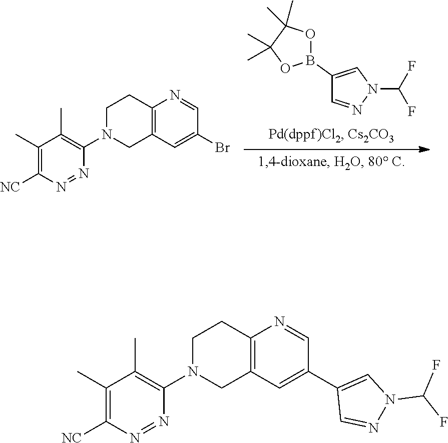 [SMILES: Cc1c(C#N)nnc(N2CCc3ncc(Br)cc3C2)c1C ; CC1(C)OB(c2cnn(C(F)F)c2)OC1(C)C ; Cc1c(C#N)nnc(N2CCc3ncc(-c4cnn(C(F)F)c4)cc3C2)c1C]

To vial under inert atmosphere was added a mixture of 1-(difluoromethyl)pyrazole-4-boronic acid pinacol ester (21 mg, 0.08 mmol), 6-(3-bromo-7,8-dihydro-1,6-naphthyridin-6(5H)-yl)-4,5-dimethylpyridazine-3-carbonitrile (15 mg, 0.04 mmol), cesium carbonate (42 mg, 0.13 mmol), and Pd(dppf)Cl₂ (3.1 mg, 0.004 mmol) in 1,4-dioxane (1.0 mL) and water (0.03 mL). The vial was sealed, and the reaction was heated to 80° C. for 1.5 h. The reaction mixture was filtered over Celite®, washed with DCM/EtOAc and concentrated. The residue was dissolved in DMSO (2 mL) and purified via RP-HPLC (20-70% MeCN/0.1% aqueous TFA). After a basic workup with saturated aqueous NaHCO₃ and extraction with 3:1 chloroform/IPA, the organic layers were concentrated to afford the title compound (9.1 mg). ES-MS [M+1]⁺: 382.2.

### Example 19

### 6-(3-(6,7-Dihydropyrazolo[1,5-a]pyrimidin-4(5H)-yl-5,5,6,6,7,7-d₆)-7,8-dihydro-1,6-naphthyridin-6(5H)-yl)-5-methylpyridazine-3-carbonitrile

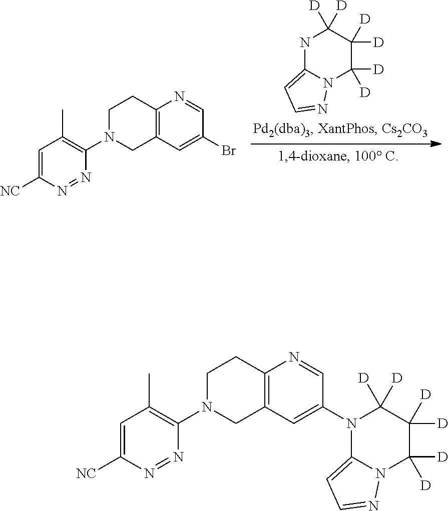 [SMILES: Cc1cc(C#N)nnc1N1CCc2ncc(Br)cc2C1 ; [2H]C1([2H])Nc2ccnn2C([2H])([2H])C1([2H])[2H] ; [2H]C1([2H])N(c2cnc3c(c2)CN(c2nnc(C#N)cc2C)CC3)c2ccnn2C([2H])([2H])C1([2H])[2H]]

To a vial under inert atmosphere was added 6-(3-bromo-7,8-dihydro-1,6-naphthyridin-6(5H)-yl)-5-methylpyridazine-3-carbonitrile (20.5 mg, 0.06 mmol), 4,5,6,7-tetrahydropyrazolo[1,5-a]pyrimidine-5,5,6,6,7,7-d₆ (8.0 mg, 0.06 mmol), cesium carbonate (41 mg, 0.12 mmol), Pd₂(dba)₃ (5.7 mg, 0.006 mmol), and XantPhos (3.6 mg, 0.006 mmol) in 1,4-dioxane (1 mL). The vial was sealed, and the mixture was heated to 100° C. for 18 h. After cooling to ambient temperature, the mixture was filtered through Celite®, washed with DCM/MeOH and concentrated. The residue was dissolved in DMSO (1 mL) and purified via RP-HPLC (5-40% MeCN/0.1% aqueous TFA). After a basic workup with saturated aqueous NaHCO₃ and extraction with 3:1 chloroform/IPA, the combined organic layers were concentrated to afford the title compound (1.3 mg). ES-MS [M+1]⁺: 379.4.

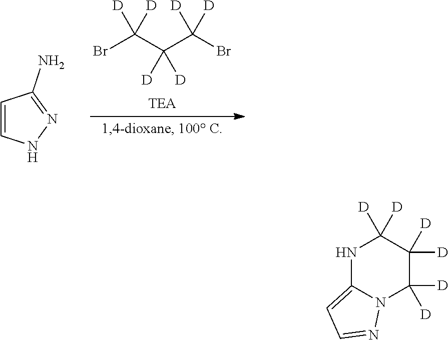 [SMILES: Nc1cc[nH]n1 ; [2H]C([2H])(Br)C([2H])([2H])C([2H])([2H])Br ; [2H]C1([2H])Nc2ccnn2C([2H])([2H])C1([2H])[2H]]

4,5,6,7-Tetrahydropyrazolo[1,5-a]pyrimidine-5,5,6,6,7,7-d₆. To a solution of 3-aminopyrazole (200 mg, 2.4 mmol) and triethylamine (670 μL, 4.8 mmol) in 1,4-dioxane (8 mL) was added 1,3-dibromopropane-d₆ (345 μL, 2.4 mmol). The mixture was heated to 100° C. for 18 h. The reaction mixture was concentrated in vacuo and purification via flash column chromatography on silica gel (0-8% MeOH/DCM with 1% NH₄OH additive) afforded the title compound (129 mg). ¹H NMR (400 MHz, DMSO-d₆) δ 7.02 (d, J=1.9 Hz, 1H), 5.89 (s, 1H), 5.15 (d, J=1.9 Hz, 1H); ES-MS [M+1]⁺: 130.2.

### Example 20

### 6-(3-(6,7-Dihydropyrazolo[1,5-a]pyrimidin-4(5H)-yl-5,5,6,6,7,7-d₆)-7,8-dihydro-1,6-naphthyridin-6(5H)-yl)-5-methylnicotinonitrile

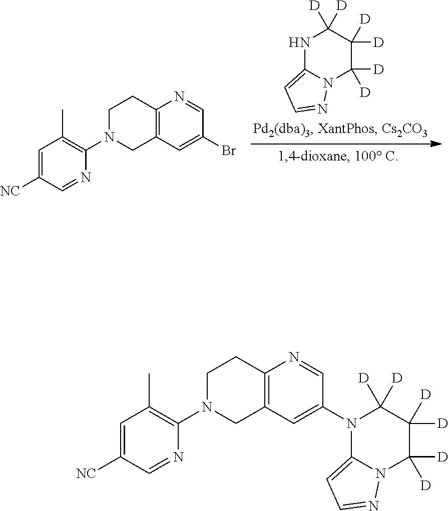 [SMILES: Cc1cc(C#N)cnc1N1CCc2ncc(Br)cc2C1 ; [2H]C1([2H])Nc2ccnn2C([2H])([2H])C1([2H])[2H] ; [2H]C1([2H])N(c2cnc3c(c2)CN(c2ncc(C#N)cc2C)CC3)c2ccnn2C([2H])([2H])C1([2H])[2H]]

### Prepared in a similar manner as example 19 to afford 6.3 mg of title compound. ES-MS [M+1]⁺: 378.2

### Example 21

(5-Methyl-6-(3-(2,2,2-trifluoroethyl)-7,8-dihydro-1,6-naphthyridin-6(5H)-yl)pyridin-3-yl)(morpholino)methanone

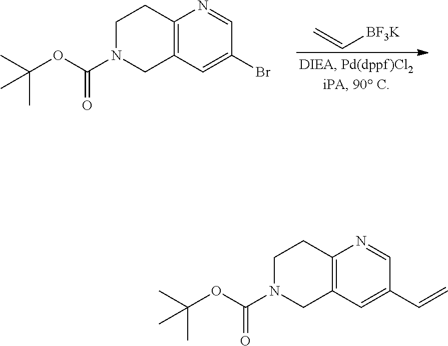

tert-Butyl 3-vinyl-7,8-dihydro-1,6-naphthyridine-6(5H)-carboxylate. To a solution of tert-butyl 3-bromo-7,8-dihydro-1,6-naphthyridine-6(5H)-carboxylate 1253 mg, 4.0 mmol) in IPA (20 mL) was added potassium vinyltrifluoroborate (804 mg, 6.0 mmol) and [1,1′-bis(diphenylphosphino)ferrocene]dichloropalladium(II) (294 mg, 0.4 mmol) and DIEA (1.4 mL, 8.0 mmol. The reaction mixture was stirred at 90° C. for 3 h. After cooling to ambient temperature, the reaction mixture was filtered through a pad of Celite® which was rinsed thoroughly with EtOAc/DCM. The filtrate was concentrated under reduced pressure. The crude material was purified using normal phase chromatography on silica gel (0-70% EtOAc/hexanes) to provide the title compound (950 mg). ¹H NMR (400 MHz, CDCl₃) δ 8.42 (d, J=2.1 Hz, 1H), 7.48 (d, J=2.1 Hz, 1H), 6.67 (dd, J=17.6, 11.0 Hz, 1H), 5.80 (d, J=17.6 Hz, 1H), 5.36 (d, J=11.0 Hz, 1H), 4.60 (s, 2H), 3.75 (t, J=6.0 Hz, 2H), 3.01 (t, J=6.0 Hz, 2H), 1.49 (s, 9H); ES-MS [M+1]⁺: 261.4.

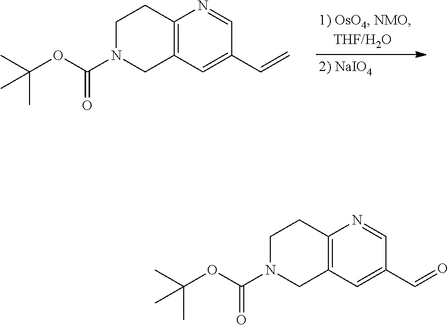

tert-Butyl 3-formyl-7,8-dihydro-1,6-naphthyridine-6(5H)-carboxylate. To a suspension of tert-butyl 3-vinyl-7,8-dihydro-1,6-naphthyridine-6(5H)-carboxylate (950 mg, 3.65 mmol) in THE (9.1 mL) and H₂O (9.1 mL) was added a solution of osmium tetraoxide (2.5 wt. % in tert-butanol, 2.30 mL) followed by 4-methylmorpholine N-oxide (513 mg, 4.38 mmol). The reaction mixture was stirred at rt for 1 h. Diol intermediate formation was detected by ES-MS [M+1]⁺: 295.4. To the mixture, sodium periodate (1.69 g, 9.12 mmol) was added. After 1 h at rt, the mixture was diluted with EtOAc and washed with 10% Na₂S₂O₃ solution (2×). The aqueous layer was extracted with EtOAc (3×). The combined extracts were washed with brine, dried over Na₂SO₄, filtered, and concentrated. Purification using normal phase chromatography on silica gel (0-100% EtOAc/hexanes) provided the title compound (850 mg). ¹H NMR (400 MHz, DMSO) δ 10.06 (s, 1H), 8.88 (d, J=2.0 Hz, 1H), 8.11-8.06 (m, 1H), 4.64 (s, 2H), 3.69 (t, J=6.0 Hz, 2H), 2.97 (t, J=6.0 Hz, 2H), 1.43 (s, 9H); ES-MS [M+1]⁺: 263.1.

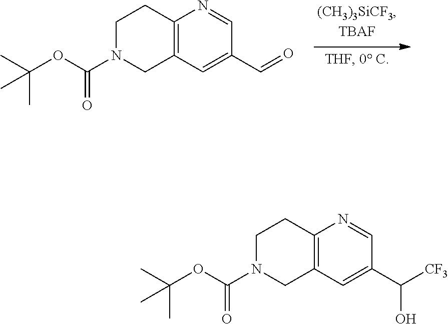 [SMILES: CC(C)(C)OC(=O)N1CCc2ncc(C=O)cc2C1 ; CC(C)(C)OC(=O)N1CCc2ncc(C(O)C(F)(F)F)cc2C1]

tert-Butyl 3-(2,2,2-trifluoro-1-hydroxyethyl)-7,8-dihydro-1,6-naphthyridine-6(5H)-carboxylate. To a solution of tert-butyl 3-formyl-7,8-dihydro-1,6-naphthyridine-6(5H)-carboxylate (430 mg, 1.64 mmol) in THE (11.0 mL) at 0° C. was added (trifluoromethyl)trimethylsilane (0.36 mL, 1.5 mmol) followed by the addition of tetrabutylammonium fluoride solution (1.0 M in THF, 2.5 mL, 2.5 mmol). After 5 min, the reaction mixture was diluted with EtOAc and washed with 10% Na₂S₂O₃ solution. The aqueous layer was re-extracted with EtOAc (3×). The combined organic layers were washed with brine, dried over Na₂SO₄, filtered and concentrated. Purification using normal phase chromatography on silica gel (0-80% EtOAc/hexanes) provided the title compound (333 mg). ¹H NMR (400 MHz, DMSO) δ 8.48-8.43 (m, 1H), 7.69 (d, J=2.1 Hz, 1H), 7.01 (s, 1H), 5.24 (q, J=7.5 Hz, 1H), 4.56 (s, 2H), 3.66 (t, J=6.0 Hz, 2H), 2.88 (t, J=6.0 Hz, 2H), 1.43 (s, 9H); ES-MS [M+1]⁺: 333.4.

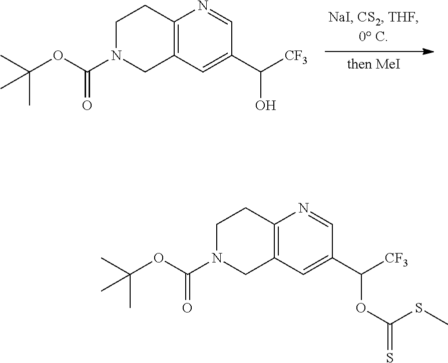 [SMILES: CC(C)(C)OC(=O)N1CCc2ncc(C(O)C(F)(F)F)cc2C1 ; CSC(=S)OC(c1cnc2c(c1)CN(C(=O)OC(C)(C)C)CC2)C(F)(F)F]

tert-Butyl 3-(2,2,2-trifluoro-1-(((methylthio)carbonothioyl)oxy)ethyl)-7,8-dihydro-1,6-naphthyridine-6(5H)-carboxylate. To a solution of tert-butyl 3-(2,2,2-trifluoro-1-hydroxyethyl)-7,8-dihydro-1,6-naphthyridine-6(5H)-carboxylate (333 mg, 1.0 mmol) in THF (10.0 mL) at 0° C. was added sodium hydride (60% dispersion in mineral oil, 100 mg, 2.51 mmol). After 1 h, a solution of carbon disulfide (5 M in THF, 0.40 mL, 2.0 mmol) was added dropwise. The mixture was allowed to stir at 0° C. for 1 h and iodomethane (0.13 mL, 2.0 mmol) was added. After 2 h at rt, the reaction mixture was quenched with ice-cold water and extracted with DCM (2×). The combined extracts were dried over Na₂SO₄, filtered, and concentrated. The crude material was used in the next step without any further purification (423 mg). ES-MS [M+1]⁺: 423.2.

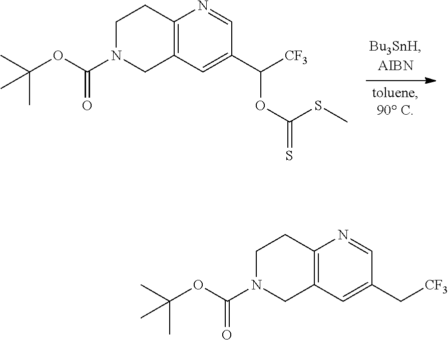

tert-Butyl 3-(2,2,2-trifluoroethyl)-7,8-dihydro-1,6-naphthyridine-6(5H)-carboxylate. To a solution of tert-butyl 3-(2,2,2-trifluoro-1-(((methylthio)carbonothioyl)oxy)ethyl)-7,8-dihydro-1,6-naphthyridine-6(5H)-carboxylate (423 mg, 1.0 mmol) in toluene (10.0 mL) was added tributyltin hydride (0.67 mL, 2.5 mmol) followed by 2,2-azobis(2-methylpropionitrile) (25 mg, 0.15 mmol). The reaction mixture was stirred at 90° C. After 30 min, the reaction mixture was concentrated under reduced pressure and purified using normal phase chromatography on silica gel (0-70% EtOAc/hexanes) to provide the title compound (255 mg). ¹H NMR (400 MHz, DMSO) δ 8.38-8.33 (m, 1H), 7.60 (s, 1H), 4.54 (s, 2H), 3.73-3.64 (m, 4H), 2.86 (t, J=6.0 Hz, 2H), 1.43 (s, 9H); ES-MS [M+1]⁺: 317.3.

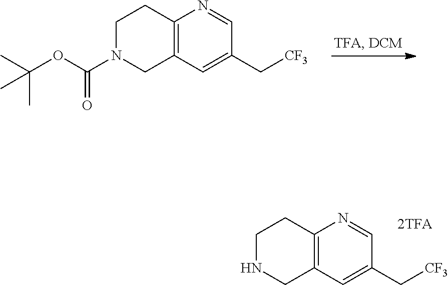 [SMILES: CC(C)(C)OC(=O)N1CCc2ncc(CC(F)(F)F)cc2C1 ; FC(F)(F)Cc1cnc2c(c1)CNCC2]

3-(2,2,2-Trifluoroethyl)-5,6,7,8-tetrahydro-1,6-naphthyridine. To a solution of tert-butyl 3-(2,2,2-trifluoroethyl)-7,8-dihydro-1,6-naphthyridine-6(5H)-carboxylate (255 mg, 0.81 mmol) in DCM (6.0 mL) was added trifluoroacetic acid (1.0 mL, 13.1 mmol). The resulting mixture was stirred at rt for 2 h and concentrated under reduced pressure. The crude material was carried forward as a TFA salt (358 mg). ES-MS [M+1]⁺: 217.3.

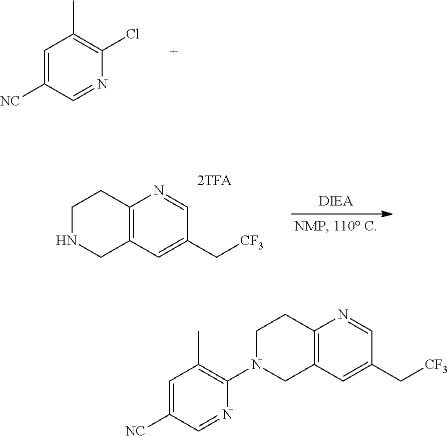

5-Methyl-6-(3-(2,2,2-trifluoroethyl)-7,8-dihydro-1,6-naphthyridin-6(5H)-yl)nicotinonitrile. Prepared in a similar manner as intermediate B to afford title compound. ¹H NMR (400 MHz, CDCl₃) δ 8.43-8.38 (m, 1H), 7.47-7.44 (m, 2H), 7.26 (s, 1H), 4.81 (s, 2H), 3.77 (t, J=5.9 Hz, 2H), 3.42 (d, J=10.6 Hz, 1H), 3.37 (d, J=10.6 Hz, 1H), 3.24 (t, J=5.9 Hz, 2H), 2.44 (d, J=0.9 Hz, 3H); ES-MS [M+1]⁺: 334.3.

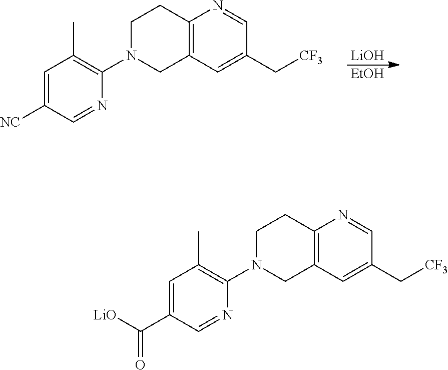

Lithium 5-methyl-6-(3-(2,2,2-trifluoroethyl)-7,8-dihydro-1,6-naphthyridin-6(5H)-yl)nicotinate. To a solution of 5-methyl-6-(3-(2,2,2-trifluoroethyl)-7,8-dihydro-1,6-naphthyridin-6(5H)-yl)nicotinonitrile (85 mg, 0.26 mmol) in ethanol (2.0 mL) was added a solution of lithium hydroxide (1M in H₂O, 1.28 mL, 1.28 mmol). The reaction mixture was stirred at 50° C. for 16 h. and concentrated under reduced pressure to provide the title compound as a lithium salt. ES-MS [M+1]⁺: 353.3.

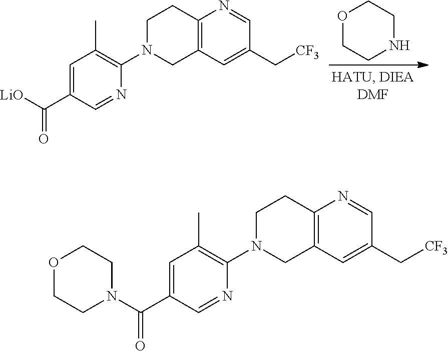

(5-Methyl-6-(3-(2,2,2-trifluoroethyl)-7,8-dihydro-1,6-naphthyridin-6(5H)-yl)pyridin-3-yl)(morpholino)methanone. To a suspension of lithium 5-methyl-6-(3-(2,2,2-trifluoroethyl)-7,8-dihydro-1,6-naphthyridin-6(5H)-yl)nicotinate (13 mg, 0.036 mmol), morpholine (15.8 mg, 0.18 mmol) and DIEA (31.5 μL, 0.18 mmol) in DMF (1.0 mL) was added HATU (28 mg, 0.072 mmol). The resulting mixture was stirred for 30 min and filtered to remove any insoluble salts. Purification using RP-HPLC to provide the title compound (4.4 mg). ES-MS [M+1]⁺: 422.4.

### Example 22

### 6-(3-Cyclopropyl-7,8-dihydro-1,6-naphthyridin-6(5H)-yl)-5-methylnicotinonitrile

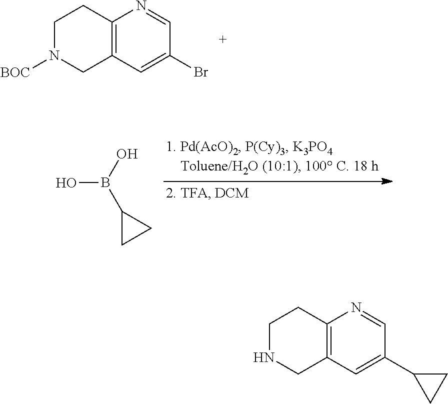 [SMILES: CC(C)(C)OC(=O)N1CCc2ncc(Br)cc2C1 ; OB(O)C1CC1 ; c1nc2c(cc1C1CC1)CNCC2]

3-Cyclopropyl-5,6,7,8-tetrahydro-1,6-naphthyridine. In a 20 mL microwave vial were combined tert-butyl 3-bromo-7,8-dihydro-5H-1,6-naphthyridine-6-carboxylate (500 mg), cyclopropylboronic acid (274 mg), Pd(AcO)₂ (36 mg), K₃PO₄ (1.03 g), tricyclohexylphosphine (90 mg) and toluene/water (10:1; 16.5 mL). The vial was capped and purged with nitrogen. The mixture was stirred at 100° C. 18 h. The reaction mixture was then filtered over Celite®, washing with EtOAc/DCM, and concentrated. The material was purified using normal phase chromatography (MeOH/DCM) to produce the BOC intermediate. The intermediate was dissolved in DCM (10 mL) and TFA (1.2 mL) was added. After 2h, solvents were removed and the crude residue was purified by SCX column (HF bond), washed with MeOH and eluted with 7NNH₃ in MeOH to produce the title product (275 mg). ES-MS [M+H]⁺=175.4.

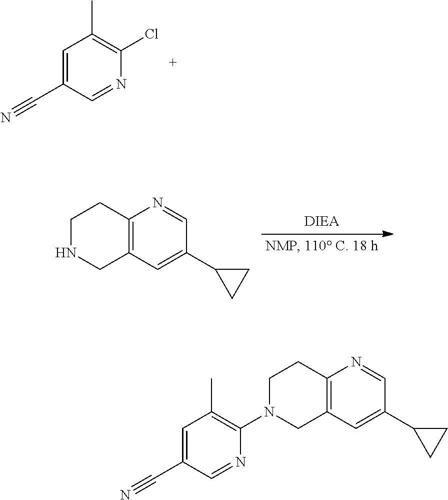

To a solution of 3-cyclopropyl-5,6,7,8-tetrahydro-1,6-naphthyridine (11 mg) in NMP (1 mL) was added N,N-diisopropylethylamine (57 μL) and 6-chloro-5-methylnicotinonitrile (10 mg) and NMP (1 mL). The mixture was stirred at 110° C. for 18h before cooling to room temperature. The reaction was then syringe filtered, diluted with DMSO, and purified by reverse phase chromatography (MeCN/H₂O/TFA). The desired fractions were neutralized with aqueous sodium bicarbonate and the MeCN was evaporated. The fractions were then diluted with water, extracted with CHCl₃/IPA (3:1) and filtered with a phase separator to produce the title compound (5.4 mg). ¹H NMR (400 MHz, CDCl₃) δ 8.42-8.37 (m, 1H), 8.26 (d, J=2.2 Hz, 1H), 7.59 (dd, J=2.2, 0.9 Hz, 1H), 7.15 (d, J=2.2 Hz, 1H), 4.54 (s, 2H), 3.64 (t, J=5.9 Hz, 2H), 3.19 (t, J=5.9 Hz, 2H), 2.36 (d, J=1.4 Hz, 3H), 1.96-1.84 (m, 1H), 1.10-0.97 (m, 2H), 0.75-0.67 (m, 2H); ES-MS [M+H]⁺=291.4.

### Example 23

### 6-(3-(2-Fluorophenoxy)-7,8-dihydro-1,6-naphthyridin-6(5H)-yl)-5-methylpyridazine-3-carbonitrile

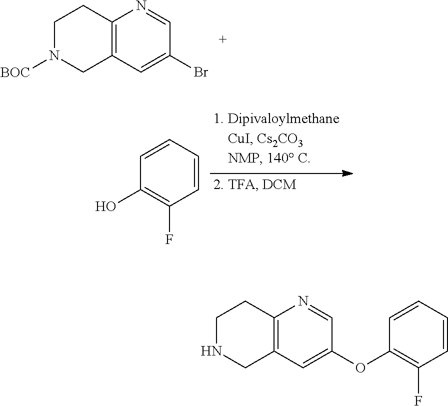 [SMILES: CC(C)(C)OC(=O)N1CCc2ncc(Br)cc2C1 ; Oc1ccccc1F ; Fc1ccccc1Oc1cnc2c(c1)CNCC2]

3-(2-Fluorophenoxy)-5,6,7,8-tetrahydro-1,6-naphthyridine. To a microwave vial were combined tert-butyl 3-bromo-7,8-dihydro-5H-1,6-naphthyridine-6-carboxylate (3.0 g), 2-fluorophenol (2.15 g), Cs₂CO₃ (6.28 g), 2,2,6,6-tetramethyl-3,5-heptanedione (200 μL), and CuI (91.0 mg). The solids were degassed followed by addition of degassed NMP (48 mL). The resulting solution was stirred at 140° C. The reaction was filtered over Celite®, washed with DCM, and concentrated in vacuo. The resulting solution was filtered, concentrated, and purified by reverse phase chromatography (5-75% MeCN/0.1% aqueous TFA). The desired fractions were concentrated to give the BOC intermediate. The intermediate was taken up in DCM (48 mL) and TFA (7.34 mL) and stirred 2 h. The reaction mixture was concentrated and purified by SCX cartridge (HF bond), washed with MeOH, and eluted with 7NNH₃ in MeOH. The filtrate was concentrated to give the title compound (950 mg). ¹H NMR (400 MHz, DMSO) δ 8.16 (d, J=2.9 Hz, 1H), 7.26-7.15 (m, 4H), 7.12 (d, J=2.8 Hz, 1H), 3.89 (s, 2H), 3.09 (t, J=6.0 Hz, 2H), 2.80 (t, J=6.1 Hz, 2H); ES-MS [M+H]⁺=245.4.

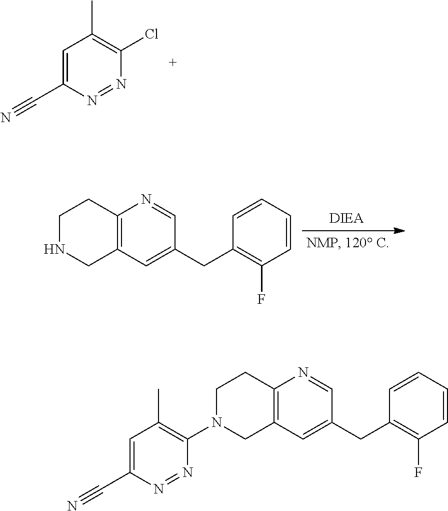

6-(3-(2-Fluorophenoxy)-7,8-dihydro-1,6-naphthyridin-6(5H)-yl)-5-methylpyridazine-3-carbonitrile. To a solution of 3-(2-fluorophenoxy)-5,6,7,8-tetrahydro-1,6-naphthyridine (11 mg) in NMP (0.5 mL) was added N,N-diisopropylethylamine (25 μL) and 6-chloro-5-methyl-pyridazine-3-carbonitrile (8 mg) and NMP (0.5 mL). The mixture was stirred at 120° C. for 30 min in a microwave reactor. The reaction was then syringe filtered, diluted with DMSO, and purified by reverse phase chromatography (5-75% MeCN/0.1% aqueous TFA). The desired fractions were neutralized with aqueous sodium bicarbonate and the fractions were then diluted with water, extracted with CHCl₃/IPA (3:1) and filtered with a phase separator to provide the title compound (9.2 mg). ¹H NMR (400 MHz, DMSO) δ 8.22 (d, J=2.8 Hz, 1H), 7.96 (d, J=1.0 Hz, 1H), 7.45-7.39 (m, 1H), 7.37 (d, J=2.8 Hz, 1H), 7.32-7.17 (m, 3H), 4.73 (s, 2H), 3.80 (t, J=5.9 Hz, 2H), 3.07 (t, J=5.9 Hz, 2H), 2.39 (d, J=0.9 Hz, 3H); ES-MS [M+H]⁺=362.4.

### Example 24

### 6-(6-Chloro-2-(methoxymethyl)-5-methylpyrimidin-4-yl)-3-(1,3-dimethyl-1H-pyrazol-4-yl)-5,6,7,8-tetrahydro-1,6-naphthyridine

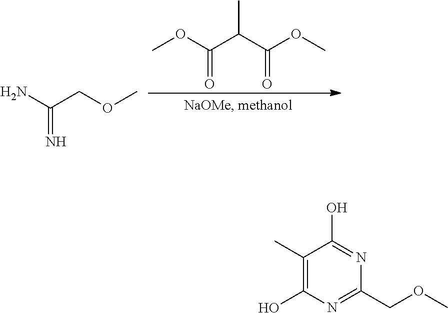

2-(Methoxymethyl)-5-methylpyrimidine-4,6-diol. A mixture of 2-methoxyacetamidine (881 mg, 10 mmol) and dimethyl methylmalonate (2.0 mL, 15 mmol) and sodium methoxide (6.86 mL, 30 mmol) in methanol (10 mL) was heated at reflux for 16 h. The crude reaction mixture was concentrated under reduced pressure to provide the title compound (1.7 g) which was carried forward without further purification. ES-MS [M+1]⁺: 171.4.

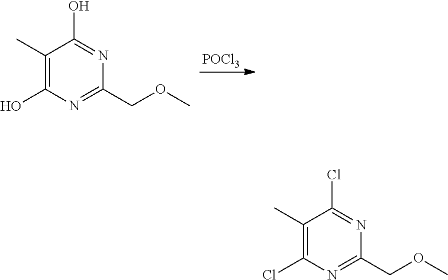

4,6-Dichloro-2-(methoxymethyl)-5-methylpyrimidine. A solution mixture of 2-(methoxymethyl)-5-methylpyrimidine-4,6-diol (1.7 g, 10 mmol) and phosphorus(V)oxychloride (25 mL) was stirred at 90° C. for 3 h. After cooling to rt, the reaction mixture was poured into a mixture of ice/water and extracted with EtOAc (3×). The combined extracts were washed with brine, dried over Na₂SO₄, filtered, and concentrated. Purification using normal phase chromatography on silica gel (0-60% EtOAc/hexanes) provided the title compound (1145 mg, 55% yield). ¹H NMR (400 MHz, CDCl₃) δ 4.60 (s, 2H), 3.52 (s, 3H), 2.48 (s, 3H); ES-MS [M+1]⁺: 207.2/209.2.

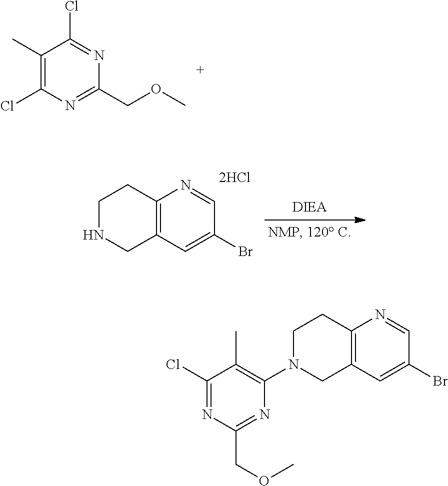

3-Bromo-6-(6-chloro-2-(methoxymethyl)-5-methylpyrimidin-4-yl)-5,6,7,8-tetrahydro-1,6-naphthyridine. 4,6-Dichloro-2-(methoxymethyl)-5-methylpyrimidine (43 mg, 0.21 mmol), 3-bromo-5,6,7,8-tetrahydro-1,6-naphthyridine (65 mg, 0.22 mmol) and DIEA (108 μL, 0.62 mmol) were combined in NMP (0.5 mL). The mixture was subjected to microwave radiation at 120° C. for 45 min. Purification using reverse phase HPLC provided the title compound (58 mg, 73% yield). ¹H NMR (400 MHz, CDCl₃) δ 8.53 (d, J=2.2 Hz, 1H), 7.72 (d, J=2.0 Hz, 1H), 4.64 (s, 2H), 4.50 (s, 2H), 3.76-3.68 (m, 3H), 3.51 (s, 3H), 3.21 (t, J=5.9 Hz, 2H), 2.33 (s, 3H); ES-MS [M+1]⁺: 385.0.

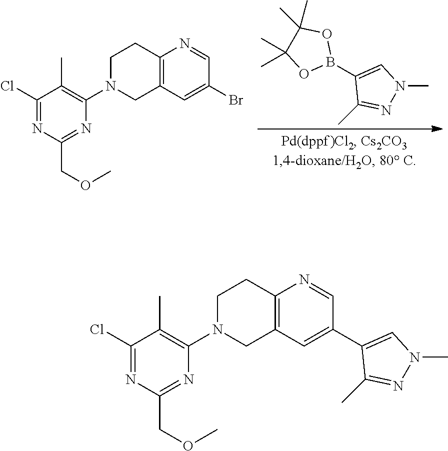

6-(6-Chloro-2-(methoxymethyl)-5-methylpyrimidin-4-yl)-3-(1,3-dimethyl-1H-pyrazol-4-yl)-5,6,7,8-tetrahydro-1,6-naphthyridine. 3-Bromo-6-(6-chloro-2-(methoxymethyl)-5-methylpyrimidin-4-yl)-5,6,7,8-tetrahydro-1,6-naphthyridine (15 mg, 0.04 mmol), 1,3-dimethyl-4-(4,4,5,5-tetramethyl-1,3,2-dioxaborolan-2-yl)-1H-pyrazole (11 mg, 0.05 mmol), cesium carbonate (27 mg, 0.08 mmol), [1,1′-bis(diphenylphosphino)ferrocene]dichloropalladium(II) (8 mg, 0.01 mmol) were combined. Anhydrous 1,4-dioxane (0.5 mL) and water (0.1 mL) were added. The resulting mixture was evacuated and purged with nitrogen (3×) and stirred at 80° C. for 16 h. Upon cooling to rt, the reaction mixture was filtered through a pad of Celite®, rinsed with EtOAc/DCM and concentrated. Purification using reverse phase HLPC provided the title compound. ES-MS [M+1]⁺: 399.2.

### Example 25

### (2,5-Dimethyl-6-(3-(trifluoromethyl)-7,8-dihydro-1,6-naphthyridin-6(5H)-yl)pyrimidin-4-yl)(morpholino)methanone

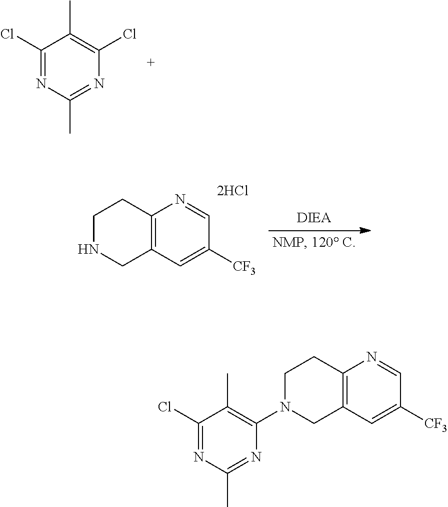

6-(6-Chloro-2,5-dimethylpyrimidin-4-yl)-3-(trifluoromethyl)-5,6,7,8-tetrahydro-1,6-naphthyridine. 4,6-Dichloro-2,5-dimethylpyrimidine (35.4 mg, 0.2 mmol), 3-(trifluoromethyl)-5,6,7,8-tetrahydro-1,6-naphthyridine dihydrochloride (60.5 mg, 0.20 mmol) and DIEA (105 μL, 0.6 mmol) were combined in NMP (1.0 mL). The reaction mixture was subjected to microwave radiation at 120° C. for 45 min. Purification using reverse phase HPLC provided the title compound (55 mg, 81% yield). ¹H NMR (400 MHz, CDCl₃) δ 8.72 (s, 1H), 7.75 (s, 1H), 4.67 (s, 2H), 3.72 (t, J=5.9 Hz, 2H), 3.28 (t, J=6.0 Hz, 2H), 2.54 (s, 3H); ES-MS [M+1]⁺: 343.2.

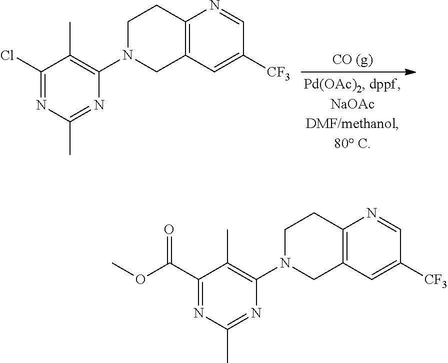 [SMILES: Cc1nc(Cl)c(C)c(N2CCc3ncc(C(F)(F)F)cc3C2)n1 ; COC(=O)c1nc(C)nc(N2CCc3ncc(C(F)(F)F)cc3C2)c1C]

Methyl 2,5-dimethyl-6-(3-(trifluoromethyl)-7,8-dihydro-1,6-naphthyridin-6(5H)-yl)pyrimidine-4-carboxylate. To a solution of 6-(6-chloro-2,5-dimethylpyrimidin-4-yl)-3-(trifluoromethyl)-5,6,7,8-tetrahydro-1,6-naphthyridine (35 mg, 0.1 mmol) and sodium acetate (85 mg, 1.02 mmol) in anhydrous methanol (2.0 mL) and DMF (0.7 mL) was added 1,1′-bis(diphenylphosphino)ferrocene (11 mg, 0.02 mmol) and palladium(II)acetate (4.6 mg, 0.02 mmol). The reaction mixture was stirred at 80° C. under carbon monoxide atmosphere. After 48 h, the mixture was filtered through a pad of Celite®, rinsed with EtOAc/DCM and concentrated. Purification using reverse phase HPLC provided the title compound (15 mg, 40% yield). ES-MS [M+1]⁺: 367.4.

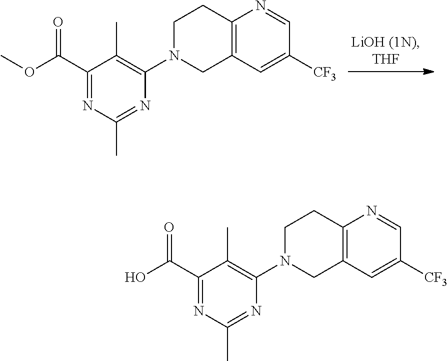 [SMILES: COC(=O)c1nc(C)nc(N2CCc3ncc(C(F)(F)F)cc3C2)c1C ; Cc1nc(C(=O)O)c(C)c(N2CCc3ncc(C(F)(F)F)cc3C2)n1]

2,5-Dimethyl-6-(3-(trifluoromethyl)-7,8-dihydro-1,6-naphthyridin-6(5H)-yl)pyrimidine-4-carboxylic acid. To a solution of methyl 2,5-dimethyl-6-(3-(trifluoromethyl)-7,8-dihydro-1,6-naphthyridin-6(5H)-yl)pyrimidine-4-carboxylate (15 mg, 0.04 mmol) in THE (0.5 mL) was added a solution of aqueous lithium hydroxide (1N solution, 0.5 mL). The reaction mixture was stirred at rt overnight and concentrated under reduced pressure to provide the title compound as a lithium salt (14.4 mg). ES-MS [M+1]⁺: 353.2.

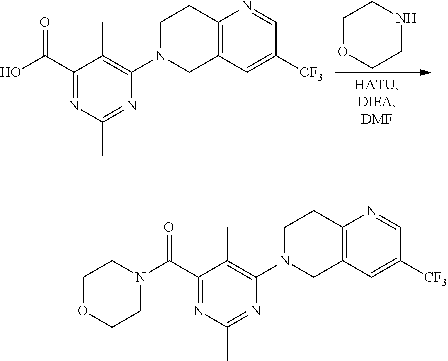

(2,5-Dimethyl-6-(3-(trifluoromethyl)-7,8-dihydro-1,6-naphthyridin-6(5H)-yl)pyrimidin-4-yl)(morpholino)methanone. 2,5-Dimethyl-6-(3-(trifluoromethyl)-7,8-dihydro-1,6-naphthyridin-6(5H)-yl)pyrimidine-4-carboxylic acid (10 mg, 0.03 mmol), HATU (22 mg, 0.06 mmol) and DIEA (25 μL, 0.14 mmol) were combined in DMF (0.5 mL). Next, morpholine (10 mg, 0.12 mmol) was added. The reaction mixture was stirred at rt for 1 h. Purification using reverse phase HLPC provided the title compound (2.2 mg). ES-MS [M+1]⁺: 422.3.

### Example 26

### 6-(6-Chloro-2,5-dimethylpyrimidin-4-yl)-8-methyl-3-nitro-5,6,7,8-tetrahydro-1,6-naphthyridine

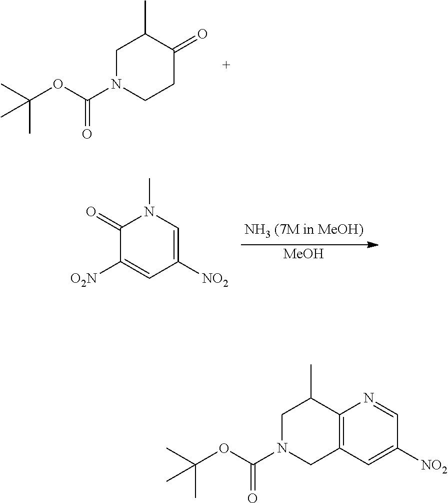 [SMILES: "MeOH" ; CC1CN(C(=O)OC(C)(C)C)CCC1=O ; Cn1cc([N+](=O)[O-])cc([N+](=O)[O-])c1=O ; CC1CN(C(=O)OC(C)(C)C)Cc2cc([N+](=O)[O-])cnc21]

tert-Butyl 8-methyl-3-nitro-7,8-dihydro-1,6-naphthyridine-6(5H)-carboxylate. To a solution of tert-butyl 3-methyl-4-oxopiperidine-1-carboxylate (2.13 g, 10 mmol) in methanol (25 mL) was added 1-methyl-3,5-dinitropyridin-2(1H)-one (2.0 g, 10 mmol) followed by a solution of NH₃ (7M in MeOH, 25 mL). The resulting mixture was divided equally into 4 microwave vials and subjected to microwave radiation at 120° C. for 1 h. The reaction mixture was concentrated under reduced pressure. Purification using normal phase chromatography on silica gel (0-100% EtOAc/hexanes) provided the title compound (2.6 g, 90% yield). ¹H NMR (400 MHz, CDCl₃) δ 9.26 (d, J=2.5 Hz, 1H), 8.21 (dd, J=2.5, 1.2 Hz, 1H), 4.95-4.73 (m, 1H), 4.60-4.52 (m, 1H), 3.83-3.56 (m, 2H), 3.24-3.14 (m, 1H), 1.50 (s, 9H), 1.38 (d, J=7.1 Hz, 3H); ES-MS [M+1]⁺: 294.4.

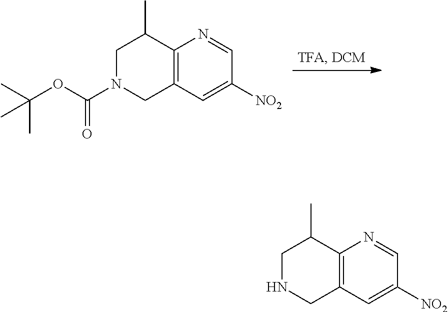 [SMILES: CC1CN(C(=O)OC(C)(C)C)Cc2cc([N+](=O)[O-])cnc21 ; CC1CNCc2cc([N+](=O)[O-])cnc21]

8-Methyl-3-nitro-5,6,7,8-tetrahydro-1,6-naphthyridine. To a solution of tert-butyl 8-methyl-3-nitro-7,8-dihydro-1,6-naphthyridine-6(5H)-carboxylate (880 mg, 3.0 mmol) in DCM (11.5 mL) was added trifluoroacetic acid (2.3 mL). The reaction mixture was stirred at rt for 6 h and concentrated under reduced pressured. The resulting residue was free-based using an Agilent HF SCX cartridge to provide the title compound (570 mg, 98% yield). ES-MS [M+1]⁺: 194.4.

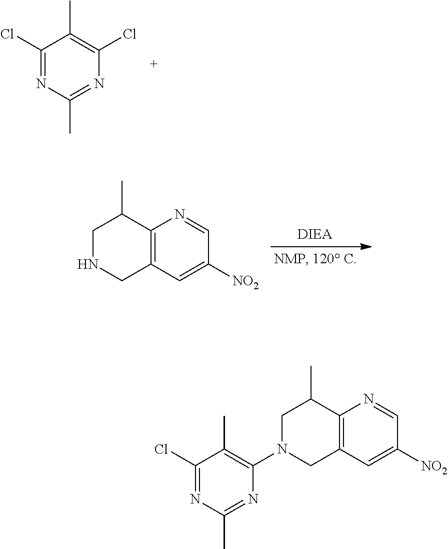

6-(6-Chloro-2,5-dimethylpyrimidin-4-yl)-8-methyl-3-nitro-5,6,7,8-tetrahydro-1,6-naphthyridine. Prepared in a similar manner as intermediate B. ES-MS [M+1]⁺: 334.4

### Example 27

### 3-(6-(3,3-Difluoropyrrolidin-1-yl)pyridin-3-yl)-6-(2,5,6-trimethylpyrimidin-4-yl)-5,6,7,8-tetrahydro-1,6-naphthyridine

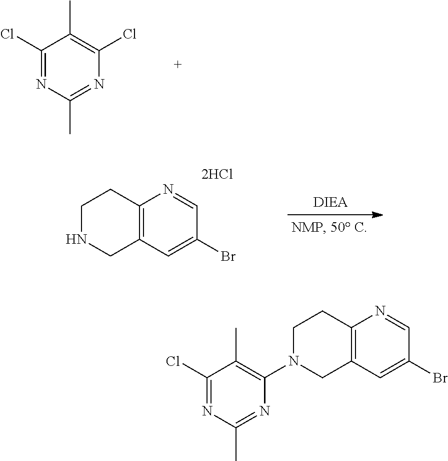

3-Bromo-6-(6-chloro-2,5-dimethylpyrimidin-4-yl)-5,6,7,8-tetrahydro-1,6-naphthyridine. Prepared in a similar manner as intermediate B. ¹H NMR (400 MHz, CDCl₃) δ 8.51 (d, J=2.2 Hz, 1H), 7.66 (d, J=2.2 Hz, 1H), 4.58 (s, 2H), 3.67 (t, J=5.9 Hz, 2H), 3.15 (t, J=5.9 Hz, 2H), 2.53 (s, 3H), 2.30 (s, 3H); ES-MS [M+1]⁺: 334.4.

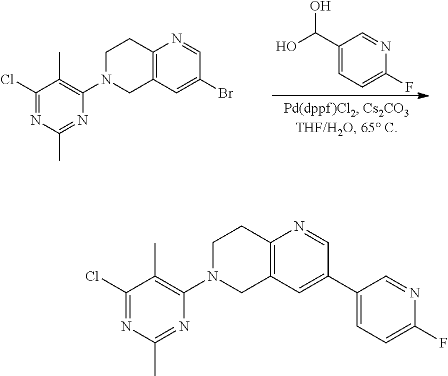 [SMILES: Cc1nc(Cl)c(C)c(N2CCc3ncc(Br)cc3C2)n1 ; OC(O)c1ccc(F)nc1 ; Cc1nc(Cl)c(C)c(N2CCc3ncc(-c4ccc(F)nc4)cc3C2)n1]

6-(6-Chloro-2,5-dimethylpyrimidin-4-yl)-3-(6-fluoropyridin-3-yl)-5,6,7,8-tetrahydro-1,6-naphthyridine. 3-Bromo-6-(6-chloro-2,5-dimethylpyrimidin-4-yl)-5,6,7,8-tetrahydro-1,6-naphthyridine (175 mg, 0.5 mmol), (6-fluoropyridin-3-yl)boronic acid (84 mg, 0.6 mmol), cesium carbonate (322 mg, 1.0 mmol), [1,1′-bis(diphenylphosphino)ferrocene]dichloropalladium(II) (54 mg, 0.07 mmol) were combined. Anhydrous THE (4.2 mL) and water (0.8 mL) were added. The resulting mixture was evacuated and purged with nitrogen (3×) and stirred at 65° C. for 1 h. Upon cooling to rt, the reaction mixture was filtered through a pad of Celite®, rinsed with EtOAc/DCM, and concentrated. Purification using normal phase chromatography on silica gel (0-80% EtOAc/hexanes) provided the title compound (135 mg, 74% yield). ¹H NMR (400 MHz, CDCl₃) δ 8.66 (s, 1H), 8.43 (s, 1H), 8.03-7.96 (m, 1H), 7.70 (s, 1H), 7.07 (d, J=5.0 Hz, 1H), 4.70 (s, 2H), 3.81-3.79 (m, 2H), 3.37-3.28 (m, 2H), 2.55 (s, 3H), 2.33 (s, 3H); ES-MS [M+1]⁺: 370.3.

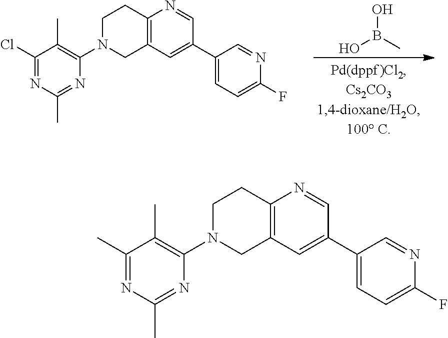

3-(6-Fluoropyridin-3-yl)-6-(2,5,6-trimethylpyrimidin-4-yl)-5,6,7,8-tetrahydro-1,6-naphthyridine. 6-(6-Chloro-2,5-dimethylpyrimidin-4-yl)-3-(6-fluoropyridin-3-yl)-5,6,7,8-tetrahydro-1,6-naphthyridine (37 mg, 0.1 mmol), methylboronic acid (30 mg, 0.5 mmol), cesium carbonate (98 mg, 0.3 mmol), [1,1′-bis(diphenylphosphino)ferrocene]dichloropalladium(II) (11 mg mg, 0.015 mmol) were combined. Anhydrous 1,4-dioxane (1.0 mL) and water (0.4 mL) were added. The resulting mixture was evacuated and purged with nitrogen (3×) and stirred at 100° C. for 1 h. Upon cooling to rt, the reaction mixture was filtered through a pad of Celite®, rinsed with EtOAc/DCM and concentrated. Purification using reverse phase HPLC provided the title compound (20 mg, 56% yield). ¹H NMR (400 MHz, CDCl₃) δ 8.65 (d, J=1.9 Hz, 1H), 8.42 (d, J=1.9 Hz, 1H), 7.97 (ddd, J=10.0, 8.0, 4.0 Hz, 1H), 7.65 (d, J=2.2 Hz, 1H), 7.07 (dd, J=8.4, 3.0 Hz, 1H), 4.76 (s, 2H), 3.85-3.75 (m, 2H), 3.27 (t, J=5.7 Hz, 2H), 2.66 (s, 3H), 2.55 (s, 3H), 2.26 (s, 3H); ES-MS [M+1]⁺: 350.4.

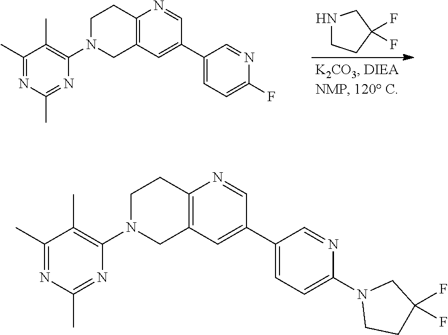 [SMILES: Cc1nc(C)c(C)c(N2CCc3ncc(-c4ccc(F)nc4)cc3C2)n1 ; FC1(F)CCNC1 ; Cc1nc(C)c(C)c(N2CCc3ncc(-c4ccc(N5CCC(F)(F)C5)nc4)cc3C2)n1]

3-(6-(3,3-Difluoropyrrolidin-1-yl)pyridin-3-yl)-6-(2,5,6-trimethylpyrimidin-4-yl)-5,6,7,8-tetrahydro-1,6-naphthyridine. 3-(6-Fluoropyridin-3-yl)-6-(2,5,6-trimethylpyrimidin-4-yl)-5,6,7,8-tetrahydro-1,6-naphthyridine (10 mg, 0.03 mmol), 3,3-difluoropyrrolidine (20.5 mg, 0.14 mmol), potassium carbonate (12 mg, 0.08 mmol) and DIEA (25 μL, 0.14 mmol) were combined in NMP (0.5 mL). The resulting mixture was stirred at 120° C. for 16 h. Purification using reverse phase HPLC provided the title compound (3.3 mg). ES-MS [M+1]⁺: 437.4.

### Example 28

### 6-(6-Chloro-5-methyl-2-(trifluoromethyl)pyrimidin-4-yl)-3-(trifluoromethyl)-5,6,7,8-tetrahydro-1,6-naphthyridine

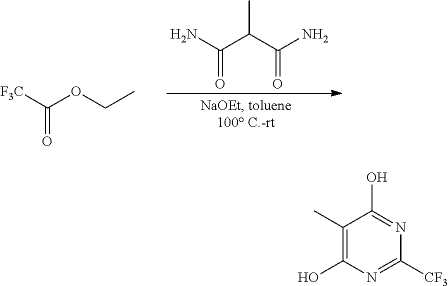

5-Methyl-2-(trifluoromethyl)pyrimidine-4,6-diol. To a suspension of 2-methylmalonamide (5.8 g, 50 mmol) in toluene (250 mL) was added a solution of sodium ethoxide (21 wt % in ethanol, 56.0 mL, 150 mmol) followed by ethyl trifluoroacetate (6.0 mL, 50 mmol). The resulting mixture was stirred at 100° C. for 3 h then allowed to cool to rt. The reaction mixture was extracted with water (3×). The combined aqueous layers were acidified to pH ˜1 using dropwise addition of concentrated HCl. The resulting precipitate was collected using vacuum filtration and dried in vacuo to provide the title compound (4.0 g, 41% yield). ¹H NMR (400 MHz, DMSO-d₆) δ 1.92 (s, 3H), OH protons not observable; ES-MS [M+1]⁺: 195.2.

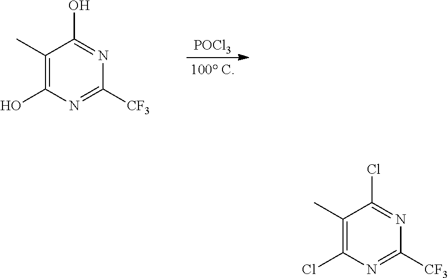

4,6-Dichloro-5-methyl-2-(trifluoromethyl)pyrimidine. To a suspension of 5-methyl-2-(trifluoromethyl)pyrimidine-4,6-diol (1.94 g, 10 mmol) in phosphorus(V)oxychloride (9.3 mL) was added triethylamine dropwise (2.8 mL, 20 mmol). The reaction mixture was stirred at 100° C. for 3 h. After cooling to rt, the reaction mixture was concentrated under reduced pressure. The resulting residue was re-dissolved in DCM and poured into a mixture of ice/water. After the layers were separated, the aqueous layer was re-extracted with DCM (3×). The combined organic layers were washed with brine, dried over Na₂SO₄, filtered and concentrated. Purification using normal phase chromatography on silica gel provided the title compound (1.2 g, 50% yield). ¹H NMR (400 MHz, CDCl₃) δ 2.58 (s, 3H); ¹³C NMR (101 MHz, CDCl₃) δ 163.33 (2C), 154.00 (q), 132.96, 118.83 (q), 16.96; ES-MS [M+1]⁺: not ionizable.

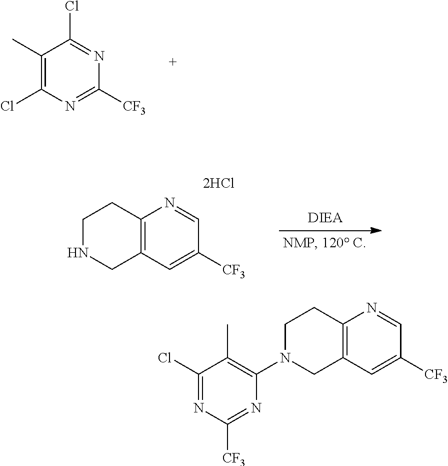 [SMILES: Cc1c(Cl)nc(C(F)(F)F)nc1Cl ; FC(F)(F)c1cnc2c(c1)CNCC2 ; Cc1c(Cl)nc(C(F)(F)F)nc1N1CCc2ncc(C(F)(F)F)cc2C1]

6-(6-Chloro-5-methyl-2-(trifluoromethyl)pyrimidin-4-yl)-3-(trifluoromethyl)-5,6,7,8-tetrahydro-1,6-naphthyridine. 4,6-Dichloro-5-methyl-2-(trifluoromethyl)pyrimidine (80 mg, 0.35 mmol) and 3-(trifluoromethyl)-5,6,7,8-tetrahydro-1,6-naphthyridine dihydrochloride (105 mg, 0.38 mmol) were combined in NMP (4.0 mL). The mixture was subjected to microwave irradiation at 120° C. for 30 min. Purification using reverse phase HPLC provided the title compound (75 mg, 55% yield). ¹H NMR (400 MHz, CDCl₃) δ 8.75 (s, 1H), 7.77 (s, 1H), 4.77 (s, 2H), 3.85 (t, J=5.9 Hz, 2H), 3.32 (t, J=5.9 Hz, 2H), 2.43 (s, 3H); ES-MS [M+1]⁺: 397.2.

### Example 29

### 6-(6-Chloro-2,5-dimethylpyrimidin-4-yl)-3-(1,3-dimethyl-1H-pyrazol-4-yl)-8,8-dimethyl-5,6,7,8-tetrahydro-1,6-naphthyridine

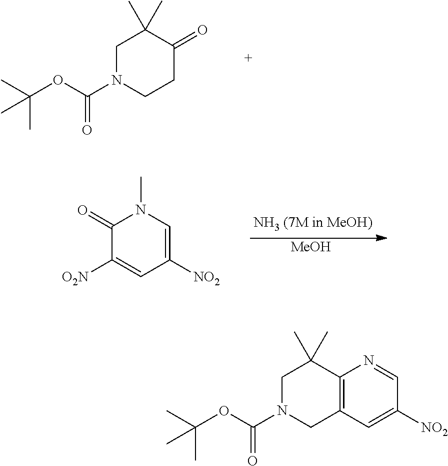 [SMILES: "MeOH" ; CC(C)(C)OC(=O)N1CCC(=O)C(C)(C)C1 ; Cn1cc([N+](=O)[O-])cc([N+](=O)[O-])c1=O ; CC(C)(C)OC(=O)N1Cc2cc([N+](=O)[O-])cnc2C(C)(C)C1]

tert-Butyl 8,8-dimethyl-3-nitro-7,8-dihydro-1,6-naphthyridine-6(5H)-carboxylate. To a solution of tert-butyl 3,3-dimethyl-4-oxopiperidine-1-carboxylate (1.8 g, 8.0 mmol) in methanol (16 mL) was added 1-methyl-3,5-dinitropyridin-2(1H)-one (1.6 g, 8.0 mmol) followed by a solution of NH₃ (7M in MeOH, 10 mL). The resulting mixture was divided equally into 4 microwave vials and subjected to microwave radiation at 120° C. for 30 min. The reaction mixture was concentrated under reduced pressure. Purification using normal phase chromatography on silica gel (0-100% EtOAc/hexanes) provided the title compound (2.4 g, 98% yield). ¹H NMR (400 MHz, CDCl₃) δ 9.26 (d, J=2.5 Hz, 1H), 8.17 (dd, J=2.5, 1.2 Hz, 1H), 4.75 (s, 2H), 3.56 (s, 2H), 1.50 (s, 9H), 1.35 (s, 6H); ES-MS [M+1]⁺: 308.4.

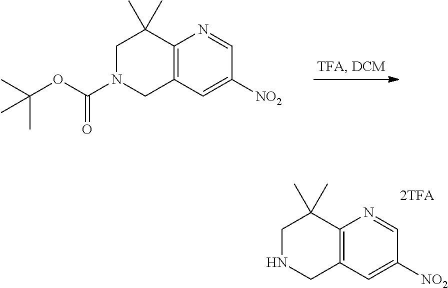 [SMILES: CC(C)(C)OC(=O)N1Cc2cc([N+](=O)[O-])cnc2C(C)(C)C1 ; CC1(C)CNCc2cc([N+](=O)[O-])cnc21]

8,8-Dimethyl-3-nitro-5,6,7,8-tetrahydro-1,6-naphthyridine. To a solution of tert-butyl 8,8-dimethyl-3-nitro-7,8-dihydro-1,6-naphthyridine-6(5H)-carboxylate (2.4 g, 7.8 mmol) in DCM (24 mL) was added trifluoroacetic acid (5.0 mL). The reaction mixture was stirred at rt for 6 h and concentrated under reduced pressured to provide the title compound as a TFA salt (3.4 g). ¹H NMR (400 MHz, DMSO-d₆) δ 9.49 (bs, 1H), 9.32 (d, J=2.6 Hz, 1H), 8.62 (d, J=2.6 Hz, 1H), 4.49 (s, 2H), 3.44 (s, 2H), 1.40 (s, 6H); ES-MS [M+1]⁺: 208.2.

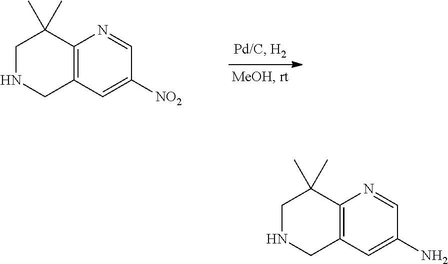

8,8-Dimethyl-5,6,7,8-tetrahydro-1,6-naphthyridin-3-amine. To a solution of 8,8-dimethyl-3-nitro-5,6,7,8-tetrahydro-1,6-naphthyridine (3.4 g, 7.8 mmol) in methanol (50 mL) under nitrogen atmosphere was added Pd/C (10 wt. % loading, matrix activated carbon support, −100 mg). The reaction mixture was stirred under hydrogen atmosphere at rt. After 16 h, the reaction mixture was filtered through a pad of Celite® which was rinsed with MeOH. The filtrate was concentrated under reduced pressure. The crude material was subjected to an Agilent HF SCX cartridge and 7N NH₃ in MeOH solution to provide the title compound (1.4 g). ES-MS [M+1]⁺: 178.4.

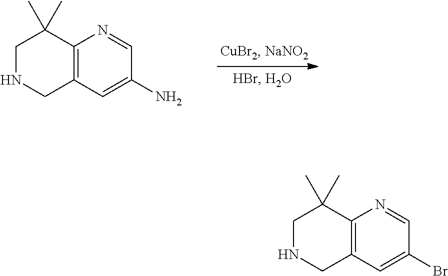

3-Bromo-8,8-dimethyl-5,6,7,8-tetrahydro-1,6-naphthyridine. A solution of 8,8-dimethyl-5,6,7,8-tetrahydro-1,6-naphthyridin-3-amine (355 mg, 2.0 mmol) in water (1.0 mL) and hydrobromic acid (1.1 mL) was cooled to −10° C., then sodium nitrite (210 mg, 3.0 mmol) was added. The reaction mixture was kept between −10° C. to −5° C. for 30 min, a solution of cupric bromide (338 mg, 1.5 mmol) in hydrobromic acid (1.1 mL) was added. The resulting mixture was slowly warmed to rt and heated at reflux for 6 h. After cooling to rt, the reaction mixture was slowly poured over a saturated solution of NaHCO₃ and extracted with EtOAc (3×). The combined extracts were washed with water, brine, dried over Na₂SO₄, filtered, and concentrated. Purification using normal phase chromatography on silica gel (10% MeOH/DCM) provided the title compound (120 mg, 25% yield). ¹H NMR (400 MHz, MeOD) δ 8.43 (d, J=2.3 Hz, 1H), 7.67 (d, J=2.3 Hz, 1H), 3.97 (s, 2H), 2.92 (s, 2H), 1.30 (s, 6H); ES-MS [M+1]⁺: 243.2.

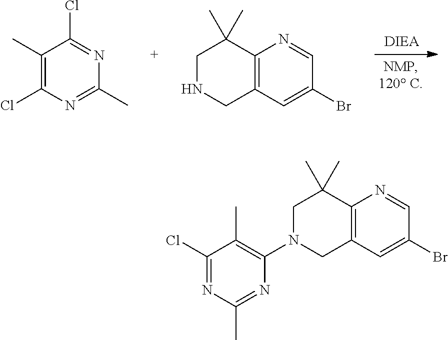

6-(6-Chloro-2,5-dimethylpyrimidin-4-yl)-8,8-dimethyl-3-(trifluoromethyl)-5,6,7,8-tetrahydro-1,6-naphthyridine. Prepared in similar manner as intermediate B. ES-MS [M+1]⁺: 383.3.

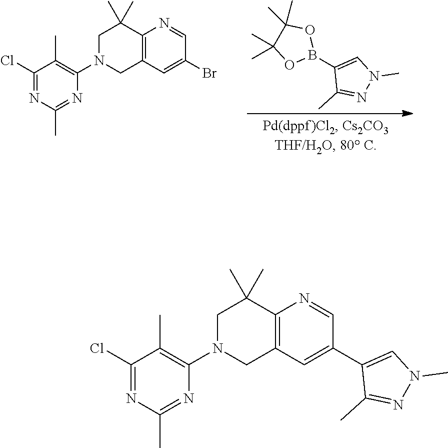

6-(6-Chloro-2,5-dimethylpyrimidin-4-yl)-3-(1,3-dimethyl-1H-pyrazol-4-yl)-8,8-dimethyl-5,6,7,8-tetrahydro-1,6-naphthyridine. Prepared in the similar manner as compound D. ES-MS [M+1]⁺: 397.4.

### Example 30

### 2-Cyclopropyl-4-(3-(trifluoromethyl)-7,8-dihydro-1,6-naphthyridin-6(5H)-yl)-5,6,7,8-tetrahydroquinazoline

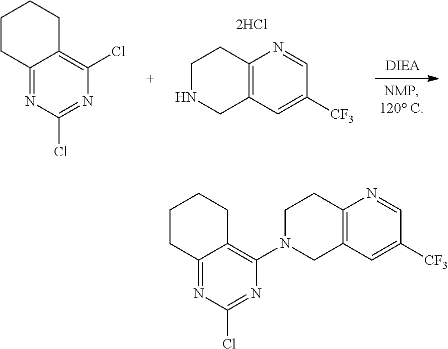 [SMILES: Clc1nc(Cl)c2c(n1)CCCC2 ; FC(F)(F)c1cnc2c(c1)CNCC2 ; FC(F)(F)c1cnc2c(c1)CN(c1nc(Cl)nc3c1CCCC3)CC2]

2-Chloro-4-(3-(trifluoromethyl)-7,8-dihydro-1,6-naphthyridin-6(5H)-yl)-5,6,7,8-tetrahydroquinazoline. To a mixture of 3-(trifluoromethyl)-5,6,7,8-tetrahydro-1,6-naphthyridine dihydrochloride (150 mg, 0.55 mmol) and 2,4-dichloro-5,6,7,8-tetrahydroquinazoline (133 mg, 0.65 mmol) in NMP (5 mL) was added DIEA (380 μL, 2.2 mmol). The reaction mixture was stirred at 70° C. for 12 h. Purification using reverse phase HPLC provided the title compound (165 mg, 82% yield). ¹H NMR (400 MHz, DMSO-d₆) δ 8.77 (d, J=2.8 Hz, 1H), 8.16 (d, J=2.8 Hz, 1H), 4.73 (s, 2H), 3.78 (t, J=5.8 Hz, 2H), 3.20-3.11 (m, 2H), 2.68 (dt, J=20.5, 6.3 Hz, 4H), 1.82-1.76 (m, 2H), 1.71-1.60 (m, 2H); ES-MS [M+1]⁺: 369.3.

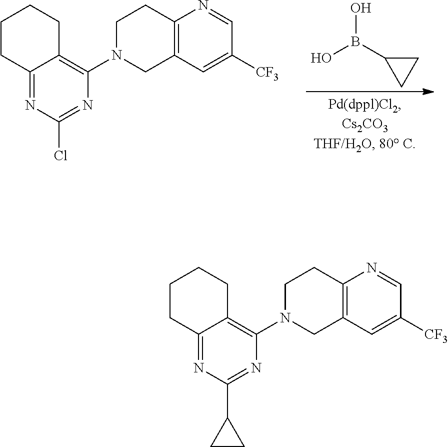 [SMILES: FC(F)(F)c1cnc2c(c1)CN(c1nc(Cl)nc3c1CCCC3)CC2 ; OB(O)C1CC1 ; FC(F)(F)c1cnc2c(c1)CN(c1nc(C3CC3)nc3c1CCCC3)CC2]

2-Cyclopropyl-4-(3-(trifluoromethyl)-7,8-dihydro-1,6-naphthyridin-6(5H)-yl)-5,6,7,8-tetrahydroquinazoline. Prepared in the similar manner as compound D. ES-MS [M+1]⁺: 375.0.

### Example 31

### 6-(2-Cyclopropyl-6,7-dihydro-5H-cyclopenta[d]pyrimidin-4-yl)-3-(trifluoromethyl)-5,6,7,8-tetrahydro-1,6-naphthyridine

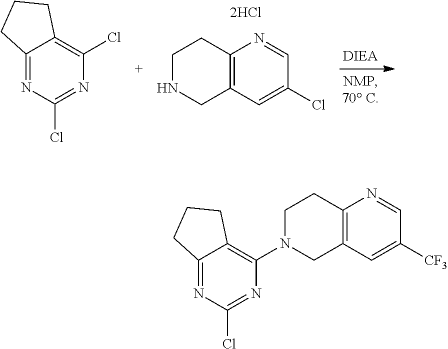 [SMILES: Clc1nc(Cl)c2c(n1)CCC2 ; Clc1cnc2c(c1)CNCC2 ; FC(F)(F)c1cnc2c(c1)CN(c1nc(Cl)nc3c1CCC3)CC2]

6-(2-Chloro-6,7-dihydro-5H-cyclopenta[d]pyrimidin-4-yl)-3-(trifluoromethyl)-5,6,7,8-tetrahydro-1,6-naphthyridine. To a mixture of 3-(trifluoromethyl)-5,6,7,8-tetrahydro-1,6-naphthyridine dihydrochloride (150 mg, 0.55 mmol) and 2,4-dichloro-6,7-dihydro-5H-cyclopenta[d]pyrimidine (124 mg, 0.65 mmol) in NMP (2.5 mL) was added DIEA (380 μL, 2.2 mmol). The reaction mixture was stirred at 70° C. overnight. The crude mixture was purified using reverse phase HPLC to afford the title compound (165 mg, 82% yield). ¹H NMR (400 MHz, DMSO-d₆) δ 8.77 (d, J=2.8 Hz, 1H), 8.22 (d, J=2.8 Hz, 1H), 4.98 (s, 2H), 4.03 (t, J=5.9 Hz, 2H), 3.17-3.06 (m, 4H), 2.75 (dt, J=20.5, 6.3 Hz, 4H), 2.05-1.97 (m, 2H), ES-MS [M+1]⁺: 355.2.

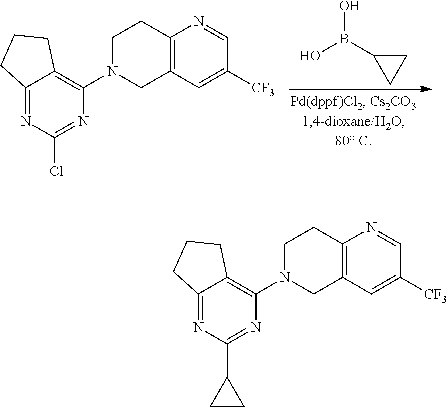 [SMILES: FC(F)(F)c1cnc2c(c1)CN(c1nc(Cl)nc3c1CCC3)CC2 ; OB(O)C1CC1 ; FC(F)(F)c1cnc2c(c1)CN(c1nc(C3CC3)nc3c1CCC3)CC2]

6-(2-Cyclopropyl-6,7-dihydro-5H-cyclopenta[d]pyrimidin-4-yl)-3-(trifluoromethyl)-5,6,7,8-tetrahydro-1,6-naphthyridine. Prepared in the similar manner as compound D. ES-MS [M+1]⁺: 361.0.

### Example 32

### 4-(4-(3-(1,3-Dimethyl-1H-pyrazol-4-yl)-7,8-dihydro-1,6-naphthyridin-6(5H)-yl)-5,6-dimethylpyrimidin-2-yl)morpholine

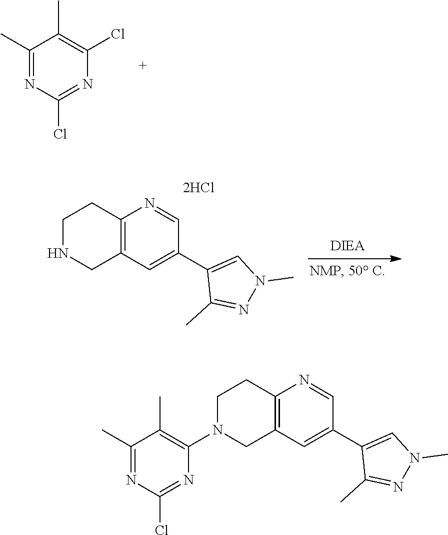

6-(2-Chloro-5,6-dimethylpyrimidin-4-yl)-3-(1,3-dimethyl-1H-pyrazol-4-yl)-5,6,7,8-tetrahydro-1,6-naphthyridine. 2,4-Dichloro-5,6-dimethylpyrimidine (71 mg, 0.40 mmol), 3-(1,3-dimethyl-1H-pyrazol-4-yl)-5,6,7,8-tetrahydro-1,6-naphthyridine dihydrochloride (80 mg, 0.27 mmol) and DIEA (185 μL, 1.1 mmol) were combined with NMP (1.3 mL). The reaction mixture was stirred at 50° C. for 1 h. The reaction mixture was purified using reverse phase HPLC to afford title compound (60 mg, 61% yield). ¹H NMR (400 MHz, CDCl₃) δ 8.50 (d, J=2.2 Hz, 1H), 7.48 (d, J=2.2 Hz, 1H), 7.46 (s, 1H), 4.62 (s, 2H), 3.90 (s, 3H), 3.67 (t, J=5.9 Hz, 2H), 3.21 (t, J=5.9 Hz, 2H), 2.43 (s, 3H), 2.23 (s, 3H); ES-MS [M+1]⁺: 369.4.

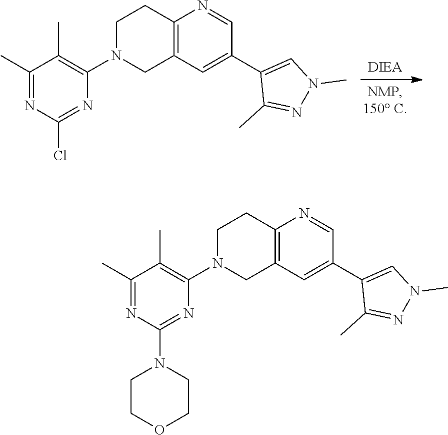

4-(4-(3-(1,3-Dimethyl-1H-pyrazol-4-yl)-7,8-dihydro-1,6-naphthyridin-6(5H)-yl)-5,6-dimethylpyrimidin-2-yl)morpholine. A mixture of 6-(2-chloro-5,6-dimethylpyrimidin-4-yl)-3-(1,3-dimethyl-1H-pyrazol-4-yl)-5,6,7,8-tetrahydro-1,6-naphthyridine (9 mg, 0.024 mmol), morpholine (21 μL, 0.24 mmol), DIEA (17 μL, 0.10 mmol) in NMP (0.6 mL) was subjected to microwave irradiation at 150° C. for 30 min. The reaction mixture was purified using reverse phase HPLC to provide the title compound (6.1 mg, 59% yield). ¹H NMR (400 MHz, DMSO-d₆) δ 8.44 (d, J=2.2 Hz, 1H), 7.95 (s, 1H), 7.64 (d, J=2.2 Hz, 1H), 4.48 (s, 2H), 3.79 (s, 3H), 3.65-3.53 (m, 8H), 3.29 (s, 2H), 3.02 (t, J=5.8 Hz, 2H), 2.29 (s, 3H), 2.23 (s, 3H), 2.08 (s, 3H); ES-MS [M+1]⁺: 420.4.

### Example 33

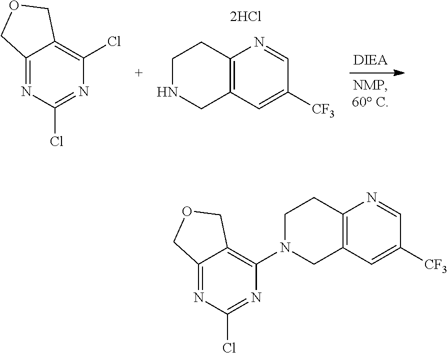 [SMILES: Clc1nc(Cl)c2c(n1)COC2 ; FC(F)(F)c1cnc2c(c1)CNCC2 ; FC(F)(F)c1cnc2c(c1)CN(c1nc(Cl)nc3c1COC3)CC2]

2-Chloro-4-(3-(trifluoromethyl)-7,8-dihydro-1,6-naphthyridin-6(5H)-yl)-5,7-dihydrofuro[3,4-d]pyrimidine. To a mixture of 3-(trifluoromethyl)-5,6,7,8-tetrahydro-1,6-naphthyridine dihydrochloride (17 mg, 0.06 mmol) and 2,4-dichloro-5,7-dihydrofuro[3,4-d]pyrimidine (10 mg, 0.05 mmol) in NMP (0.75 mL) was added DIEA (36 μL, 0.3 mmol). The reaction mixture was stirred at 60° C. for 90 min. The reaction mixture was purified using reverse phase HPLC to provide the title compound (7.6 mg, 41% yield). ¹H NMR (400 MHz, DMSO-d₆) δ 8.79 (d, J=2.8 Hz, 1H), 8.24 (d, J=2.8 Hz, 1H), 5.34 (t, J=2.7 Hz, 2H), 4.92 (s, 2H), 4.80 (t, J=2.7 Hz, 2H), 3.91 (t, J=6.0 Hz, 2H), 3.11 (t, J=5.9 Hz, 2H); ES-MS [M+1]⁺: 357.3.

### Example 34

### 1-(6-(2-Chloro-5,7-dihydrofuro[3,4-d]pyrimidin-4-yl)-5,6,7,8-tetrahydro-1,6-naphthyridin-3-yl)-2,3-dihydro-1H-pyrido[2,3-b][1,4]oxazine

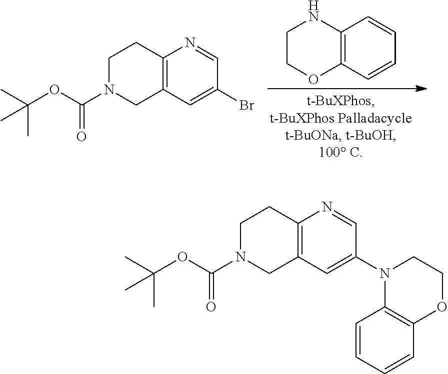 [SMILES: CC(C)(C)OC(=O)N1CCc2ncc(Br)cc2C1 ; c1ccc2c(c1)NCCO2 ; CC(C)(C)OC(=O)N1CCc2ncc(N3CCOc4ccccc43)cc2C1]

tert-Butyl 3-(2,3-dihydro-4H-benzo[b][1,4]oxazin-4-yl)-7,8-dihydro-1,6-naphthyridine-6(5H)-carboxylate. To a solution of tert-butyl 3-bromo-7,8-dihydro-1,6-naphthyridine-6(5H)-carboxylate (940 mg, 3.0 mmol) in t-BuOH (15 mL) was added 3,4-dihydro-2H-benzo[b][1,4]oxazine (490 mg, 3.6 mmol), t-BuXPhos (191 mg, 0.45 mmol), t-BuXPhos Palladacycle (310 mg, 0.15 mmol) and sodium tert-butoxide (577 mg, 6.0 mmol). The reaction mixture was evacuated and purged with nitrogen (3×) and stirred at 100° C. overnight. After cooling to ambient temperature, the reaction mixture was filtered through a pad of Celite® which was rinsed with EtOAc/DCM. The filtrate was concentrated under reduced pressure. The crude material was purified via reverse phase HPLC to provide the title compound (1.2 g, 64% yield). ES-MS [M+1]⁺: 369.3.

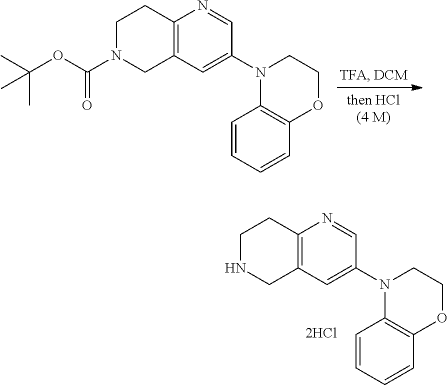

4-(5,6,7,8-Tetrahydro-1,6-naphthyridin-3-yl)-3,4-dihydro-2H-benzo[b][1,4]oxazine dihydrochloride. To a solution of tert-butyl 3-(2,3-dihydro-4H-benzo[b][1,4]oxazin-4-yl)-7,8-dihydro-1,6-naphthyridine-6(5H)-carboxylate (1.2 g, 1.92 mmol) in DCM (19 mL) was added trifluoroacetic acid (5.0 mL). The reaction mixture was stirred at rt for 4 h and concentrated under reduced pressure. The resulting residue was re-dissolved in 1,4-dioxane. A solution of HCl (4.0 M in 1,4-dioxane) was added. The mixture was sonicated for 5-10 min, concentrated under reduced pressure, and carried forward as a HCl salt (728 mg). ¹H NMR (400 MHz, DMSO-d₆) δ 9.89 (s, 1H), 8.56 (d, J=2.6 Hz, 1H), 7.91 (d, J=2.6 Hz, 1H), 7.74 (dd, J=5.0, 1.6 Hz, 1H), 7.37 (dd, J=8.0, 1.6 Hz, 1H), 6.96 (dd, J=8.0, 5.0 Hz, 1H), 4.50 (dd, J=5.2, 3.6 Hz, 2H), 4.37-4.30 (m, 2H), 3.81 (dd, J=5.2, 3.8 Hz, 2H), 3.75-3.62 (m, 1H), 3.54-3.42 (m, 1H), 3.18 (d, J=12.6 Hz, 2H); ES-MS [M+1]⁺: 269.4.

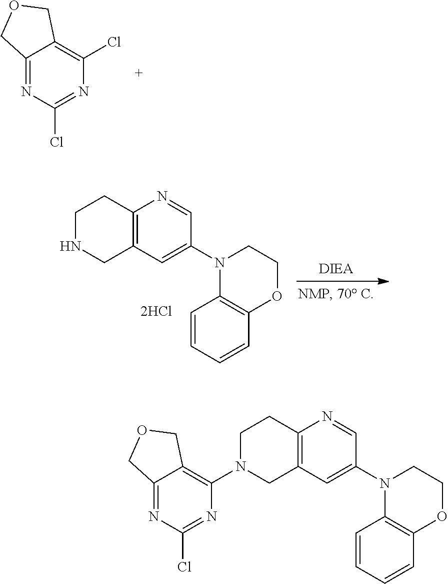 [SMILES: Clc1nc(Cl)c2c(n1)COC2 ; c1ccc2c(c1)OCCN2c1cnc2c(c1)CNCC2 ; Clc1nc2c(c(N3CCc4ncc(N5CCOc6ccccc65)cc4C3)n1)COC2]

1-(6-(2-Chloro-5,7-dihydrofuro[3,4-d]pyrimidin-4-yl)-5,6,7,8-tetrahydro-1,6-naphthyridin-3-yl)-2,3-dihydro-1H-pyrido[2,3-b][1,4]oxazine. To a mixture of 4-(5,6,7,8-tetrahydro-1,6-naphthyridin-3-yl)-3,4-dihydro-2H-benzo[b][1,4]oxazine dihydrochloride (20 mg, 0.06 mmol) and 2,4-dichloro-5,7-dihydrofuro[3,4-d]pyrimidine (10 mg, 0.05 mmol) in NMP (0.75 mL) was added DIEA (36 μL, 0.3 mmol). The reaction mixture was stirred at 70° C. for 90 min. The reaction mixture was purified using reverse phase HPLC to provide the title compound (7.8 mg, 35% yield). ¹H NMR (400 MHz, DMSO-d₆) δ 8.38 (d, J=2.6 Hz, 1H), 7.71 (d, J=2.6 Hz, 1H), 7.62 (dd, J=4.7, 1.6 Hz, 1H), 7.12 (dd, J=7.9, 1.6 Hz, 1H), 6.81 (dd, J=7.9, 4.7 Hz, 1H), 5.33 (t, J=2.7 Hz, 2H), 4.84-4.77 (m, 4H), 4.45-4.38 (m, 2H), 3.88 (d, J=6.9 Hz, 2H), 3.76-3.69 (m, 2H), 2.99 (d, J=11.9 Hz, 2H); ES-MS [M+1]⁺: 423.2

### Example 35

### 4-Benzyl-7-methyl-8-[3-(trifluoromethyl)-7,8-dihydro-5H-1,6-naphthyridin-6-yl]-2,3-dihydropyrido[3,2-f][1,4]oxazepin-5-one

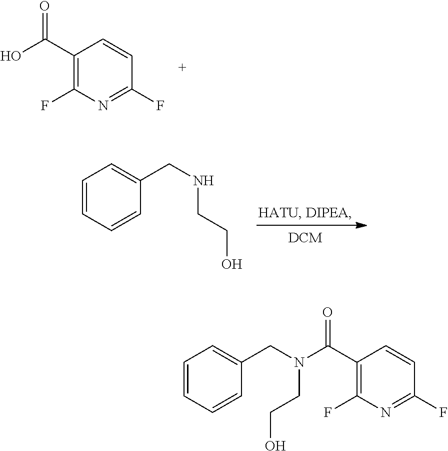 [SMILES: O=C(O)c1ccc(F)nc1F ; OCCNCc1ccccc1 ; O=C(c1ccc(F)nc1F)N(CCO)Cc1ccccc1]

N-benzyl-2,6-difluoro-N-(2-hydroxyethyl)pyridine-3-carboxamide. To a solution of 2,6-difluoronicotinic acid (500 mg) in DCM (4.7 mL) and N,N-diisopropylethylamine (1.64 mL) was added HATU (2.1 g). After 10 min, to the reaction was added N-benzylethanolamine (712 mg). The solution stirred at room temperature for 18 h. The reaction was concentrated and purified by normal-phase chromatography on silica gel (0-50% EtOAc/Hexanes) to afford the title compound (872 mg, 95% yield). ES-MS [M+1]⁺: 293.4.

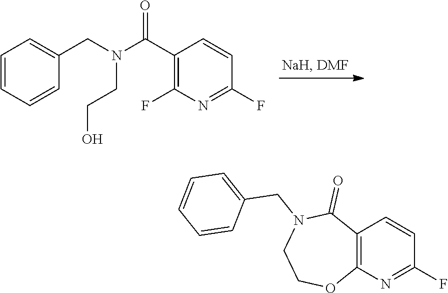 [SMILES: O=C(c1ccc(F)nc1F)N(CCO)Cc1ccccc1 ; O=C1c2ccc(F)nc2OCCN1Cc1ccccc1]

4-Benzyl-8-fluoro-2,3-dihydropyrido[3,2-f][1,4]oxazepin-5-one. To a 0° C. suspension of N-benzyl-2,6-difluoro-N-(2-hydroxyethyl)pyridine-3-carboxamide (772 mg) in DMF (13 mL) was added sodium hydride (158 mg). The reaction stirred at 0° C. for 1 h, then warmed to room temperature and stirred for 18 h. To the reaction was added water (50 mL) and diluted with EtOAc (50 mL). The layers were separated, and the organic layer was washed with water (3×), brine (2×), dried (MgSO₄), filtered, and concentrated. The crude residue was purified by normal-phase chromatography on silica gel (30-100% EtOAc/Hexanes) to afford the title compound (212 mg, 29% yield). ¹H NMR (400 MHz, CDCl₃) δ 8.66 (t, J=8.3 Hz, 1H), 7.36-7.30 (m, 5H), 6.73 (dd, J=8.4, 3.4 Hz, 1H), 4.81 (s, 2H), 4.40-4.38 (m, 2H), 3.63-3.61 (m, 2H); ES-MS [M+1]⁺: 273.2.

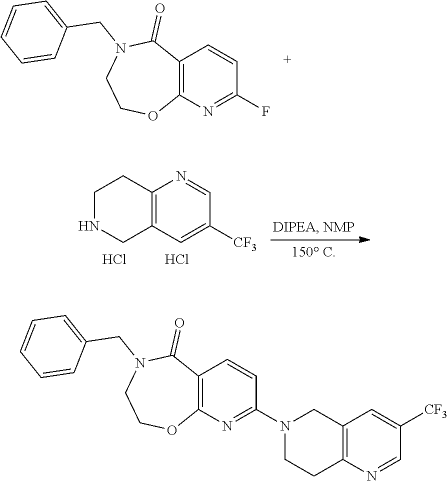 [SMILES: O=C1c2ccc(F)nc2OCCN1Cc1ccccc1 ; FC(F)(F)c1cnc2c(c1)CNCC2 ; Cl ; Cl ; O=C1c2ccc(N3CCc4ncc(C(F)(F)F)cc4C3)nc2OCCN1Cc1ccccc1]

4-Benzyl-8-[3-(trifluoromethyl)-7,8-dihydro-5H-1,6-naphthyridin-6-yl]-2,3-dihydropyrido[3,2-f][1,4]oxazepin-5-one. To a vial were added 7-(trifluoromethyl)-2,5-diazatetralin dihydrochloride (224 mg), 4-benzyl-8-fluoro-2,3-dihydropyrido[3,2-J][1,4]oxazepin-5-one (185 mg), and N,N-diisopropylethylamine (0.59 mL) in NMP (1 mL). The reaction was subjected to microwave irradiation at 150° C. for 20 min. The reaction was filtered over Celite® and purified by reverse-phase HPLC to afford the title compound (132 mg, 43% yield). ES-MS [M+1]⁺: 455.2.

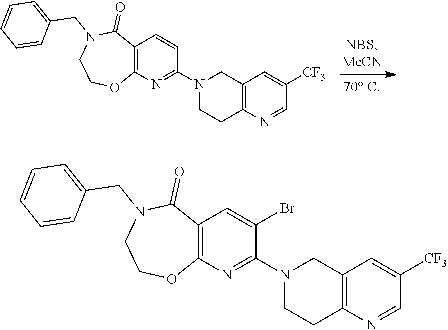 [SMILES: O=C1c2ccc(N3CCc4ncc(C(F)(F)F)cc4C3)nc2OCCN1Cc1ccccc1 ; O=C1c2cc(Br)c(N3CCc4ncc(C(F)(F)F)cc4C3)nc2OCCN1Cc1ccccc1]

4-Benzyl-7-bromo-8-[3-(trifluoromethyl)-7,8-dihydro-5H-1,6-naphthyridin-6-yl]-2,3-dihydropyrido[3,2-][1,4]oxazepin-5-one. To a solution of N-bromosuccinimide (62 mg) in MeCN (2.9 mL) was added 4-benzyl-8-[3-(trifluoromethyl)-7,8-dihydro-5H-1,6-naphthyridin-6-yl]-2,3-dihydropyrido[3,2-J][1,4]oxazepin-5-one (132 mg). The reaction was heated at 70° C. After 18 h, the solvents were removed in vacuo and the residue was diluted with DCM/Water. The layers were separated, and the organic layer was concentrated. The crude residue was purified by normal-phase chromatography on silica gel (0-50% EtOAc/Hexanes) to afford the title compound (104 mg, 67%). ¹H NMR (400 MHz, CDCl₃) δ 8.72 (s, 1H), 8.70 (s, 1H), 7.68 (s, 1H), 7.36-7.28 (m, 5H), 4.79 (s, 2H), 4.69 (s, 2H), 4.38-4.36 (m, 2H), 3.89 (t, J=5.8 Hz, 2H), 3.62-3.60 (m, 2H), 3.32 (t, J=5.6 Hz, 2H); ES-MS [M+1]⁺: 533.0/535.0.

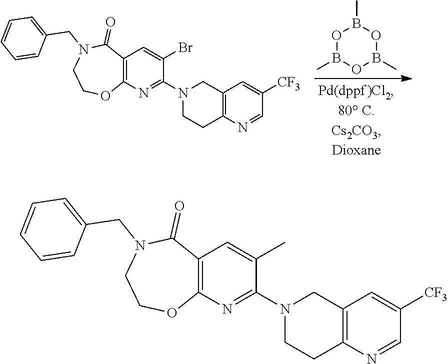

4-Benzyl-7-methyl-8-[3-(trifluoromethyl)-7,8-dihydro-5H-1,6-naphthyridin-6-yl]-2,3-dihydropyrido[3,2-f][1,4]oxazepin-5-one. To a vial were added 4-benzyl-7-bromo-8-[3-(trifluoromethyl)-7,8-dihydro-5H-1,6-naphthyridin-6-yl]-2,3-dihydropyrido[3,2-f][1,4]oxazepin-5-one (15 mg), cesium carbonate (27 mg), trimethylboroxine (16 μL), 1,4-dioxane (0.8 mL), and [1,1′-bis(diphenylphosphino)ferrocene]dichloropalladium(II) (2 mg). The mixture was evacuated and purged with nitrogen (3×). The reaction was heated to 80° C. After 5 h, the reaction was filtered over Celite® and concentrated. The crude residue was purified by reverse-phase HPLC to afford the title compound (2.8 mg, 21% yield). ¹H NMR (400 MHz, CDCl₃) δ 8.70 (s, 1H), 8.33 (s, 1H), 7.70 (s, 1H), 7.36-7.29 (m, 5H), 4.81 (s, 2H), 4.61 (s, 2H), 4.36 (t, J=3.9 Hz, 2H), 3.64-3.59 (m, 4H), 3.30-3.26 (m, 2H), 2.34 (s, 3H); ES-MS [M+1]⁺: 469.2.

### Example 36

### 5-methyl-6-(8-methyl-3-morpholino-7,8-dihydro-1,6-naphthyridin-6(5H)-yl)nicotinonitrile enantiomer synthesis

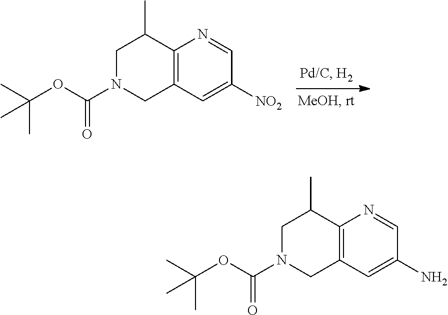 [SMILES: CC1CN(C(=O)OC(C)(C)C)Cc2cc([N+](=O)[O-])cnc21 ; CC1CN(C(=O)OC(C)(C)C)Cc2cc(N)cnc21]

Step A: tert-Butyl 3-amino-8-methyl-7,8-dihydro-1,6-naphthyridine-6(5H)-carboxylate. To a solution of tert-butyl 8-methyl-3-nitro-7,8-dihydro-1,6-naphthyridine-6(5H)-carboxylate (760 mg, 2.6 mmol) in methanol (13 mL) under nitrogen atmosphere was added Pd/C (10 wt. % loading, matrix activated carbon support, −30 mg). The reaction mixture was stirred under hydrogen atmosphere at rt. After 16 h, the reaction mixture was filtered through a pad of Celite® which was rinsed with MeOH. The filtrate was concentrated under reduced pressure. The crude material was subjected to an Agilent HF SCX cartridge and 7N NH₃ in MeOH solution to provide the title compound as a racemic mixture. ES-MS [M+1]⁺: 264.2.

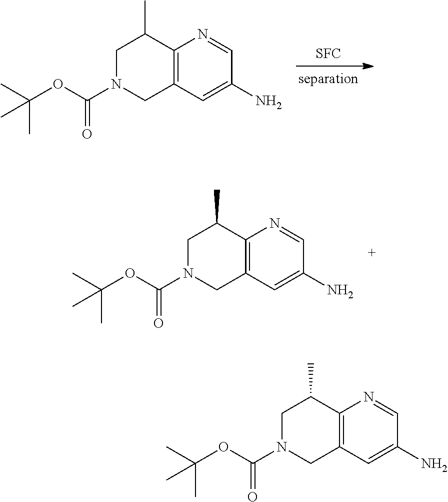 [SMILES: CC1CN(C(=O)OC(C)(C)C)Cc2cc(N)cnc21 ; "SFC" ; C[C@@H]1CN(C(=O)OC(C)(C)C)Cc2cc(N)cnc21 ; C[C@H]1CN(C(=O)OC(C)(C)C)Cc2cc(N)cnc21]

Step B: tert-butyl(R)-3-amino-8-methyl-7,8-dihydro-1,6-naphthyridine-6(5H)-carboxylate and tert-butyl(S)-3-amino-8-methyl-7,8-dihydro-1,6-naphthyridine-6(5H)-carboxylate. tert-Butyl 3-amino-8-methyl-7,8-dihydro-1,6-naphthyridine-6(5H)-carboxylate was subjected to SFC to afford enantiomerically pure title compounds.

### Analytical Separation Example

Chiral SFC separation was performed on a Thar (Waters) Investigator. Column: Chiral Technologies CHIRALPAK IA, 4.6×250 mm, 5 μm. Gradient conditions: 20-50% methanol (0.1% DEA) in CO₂ over 5 min. Flow rate: 3.5 mL/min. Column temperature: 40° C. System backpressure: 100 bar.

### Preparative Separation Example

Chiral SFC separation was performed on a PIC Solution SFC-PICLab PREP 100. Column: Chiral Technologies CHIRALPAK IA, 20×250 mm, 5 μm. Conditions: 8% isocratic methanol in CO₂. Flow rate: 80 mL/min. Column temperature: 40° C. System backpressure: 100 bar.

tert-Butyl 3-amino-8-methyl-7,8-dihydro-1,6-naphthyridine-6(5H)-carboxylate (first eluted peak):

Rt=4.16 min (analytical method); ES-MS [M+H]⁺=264.2.; 98.2% ee.

¹H NMR (400 MHz, DMSO-d₆) δ 7.78 (d, J=2.6 Hz, 1H), 6.67 (s, 1H), 5.12 (s, 2H), 4.48 (d, J=16.9 Hz, 1H), 3.57 (dd, J=13.0, 4.6 Hz, 1H), 2.77 (h, J=6.7 Hz, 1H), 1.42 (s, 9H), 1.13 (d, J=6.9 Hz, 3H), NH₂ proton —not observable.

tert-Butyl 3-amino-8-methyl-7,8-dihydro-1,6-naphthyridine-6(5H)-carboxylate (second eluted peak):

Rt=4.63 min (analytical method); ES-MS [M+H]⁺=264.2; 95.2% ee. ¹H NMR (400 MHz, DMSO-d₆) δ 7.78 (d, J=2.6 Hz, 1H), 6.64 (s, 1H), 5.12 (s, 2H), 4.48 (d, J=16.9 Hz, 1H), 3.57 (dd, J=13.1, 4.6 Hz, 1H), 2.77 (h, J=6.7 Hz, 1H), 1.42 (s, 9H), 1.13 (d, J=6.9 Hz, 3H); NH₂ proton —not observable.

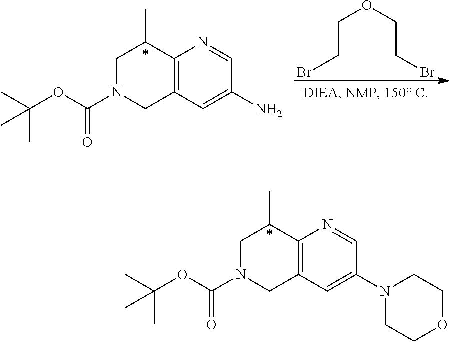 [SMILES: CC1CN(C(=O)OC(C)(C)C)Cc2cc(N)cnc21 ; BrCCOCCBr ; CC1CN(C(=O)OC(C)(C)C)Cc2cc(N3CCOCC3)cnc21]

Step C: tert-Butyl 8-methyl-3-morpholino-7,8-dihydro-1,6-naphthyridine-6(5H)-carboxylate enantiomer synthesis. A solution of the first eluting isomer of tert-butyl 3-amino-8-methyl-7,8-dihydro-1,6-naphthyridine-6(5H)-carboxylate (126 mg, 0.48 mmol) in Example 36, Step B, 1-bromo-2-(2-bromoethoxy)ethane (60 μL, 0.48 mmol) and DIEA (420 μL, 2.40 mmol) in NMP (2.0 mL) was subjected to microwave irradiation for 1 h at 150° C. The reaction mixture was purified using reverse phase HPLC to provide an enantiomer of the title compound (59 mg). ¹H NMR (400 MHz, DMSO-d₆) δ 8.16 (d, J=2.8 Hz, 1H), 7.13 (d, J=2.8 Hz, 1H), 4.59 (d, J=17.1 Hz, 1H), 3.77-3.70 (m, 4H), 3.61 (dd, J=13.1, 4.6 Hz, 1H), 3.15-3.08 (m, 4H), 2.86 (h, J=6.6 Hz, 1H), 1.42 (s, 9H), 1.17 (d, J=7.0 Hz, 3H); ES-MS [M+1]⁺

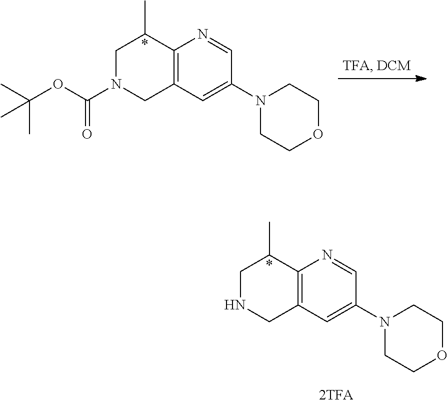 [SMILES: CC1CN(C(=O)OC(C)(C)C)Cc2cc(N3CCOCC3)cnc21 ; CC1CNCc2cc(N3CCOCC3)cnc21]

Step D: 4-(8-methyl-5,6,7,8-tetrahydro-1,6-naphthyridin-3-yl)morpholine enantiomer synthesis. To a solution of the product of Example 36, Step C (59.4 mg, 0.18 mmol) in DCM (1.0 mL) was added trifluoroacetic acid (1.0 mL). The reaction mixture was stirred at rt for 2 h and concentrated under reduced pressured. The crude residue was carried forward as a TFA salt without further purification. ¹H NMR (400 MHz, DMSO-d₆) δ 9.13 (bs, 1H), 8.30 (d, J=2.8 Hz, 1H), 7.33 (d, J=2.8 Hz, 1H), 4.34-4.24 (m, 2H), 3.79-3.72 (m, 4H), 3.63-3.56 (m, 1H), 3.22-3.11 (m, 6H), 1.33 (d, J=6.7 Hz, 3H).

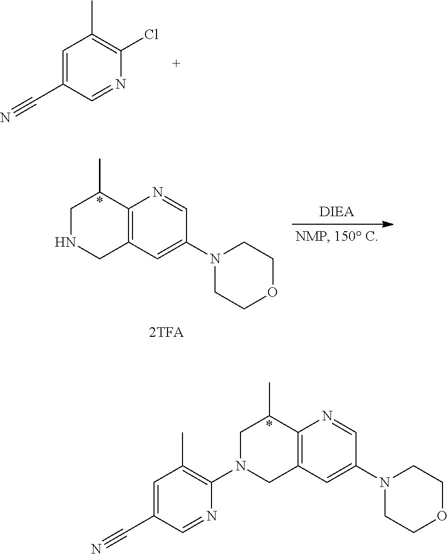

Step E: 5-methyl-6-(8-methyl-3-morpholino-7,8-dihydro-1,6-naphthyridin-6(5H)-yl)nicotinonitrile enantiomer synthesis. To a mixture of the product of Example 36, Step D (13.7 mg, 0.03 mmol) and 6-chloro-5-methylnicotinonitrile (9.1 mg, 0.06 mmol) in NMP (1 mL) was added DIEA (26 μL, 0.15 mmol). The reaction mixture was subjected to microwave irradiation at 150° C. for 30 min. The reaction mixture was purified using reverse phase HPLC to affford an enantiomer of the title compound (1.5 mg). ES-MS [M+1]⁺: 350.2.

### Example 37

### 6-(3-((3-fluoropyridin-4-yl)amino)-7,8-dihydro-1,6-naphthyridin-6(5H)-yl-5,5,7,7-d₄)-5-methylpyridazine-3-carbonitrile

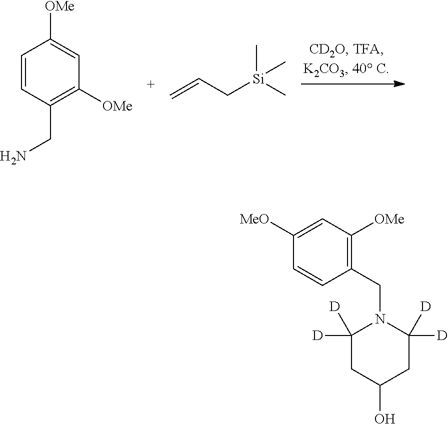

1-(2,4-Dimethoxybenzyl)piperidin-2,2,6,6-d₄-4-ol. Deuterated formaldehyde (7.58 mL, 55.0 mmol) was added to 2,4-dimethoxybenzylamine (3.64 mL, 23.9 mmol). Then, trifluoroacetic acid (1.83 mL, 23.9 mmol) was added. The resulting mixture was sonicated for 10 min and then stirred for 1 h at room temperature. To the resulting solution was added allyltrimethylsilane (4.18 mL, 26.3 mmol) and the reaction was heated at 40° C. for 18 h. The reaction was diluted with water (8 mL) and DCM (8 mL), and solid potassium carbonate (1.67 g, 12.0 mmol) was added. The mixture stirred for 10 min and was then extracted with 3:1 CHCl₃/IPA (5×). The combined organic layers were dried (MgSO₄), filtered, and concentrated. The crude oil was purified by silica gel chromatography (0-20% MeOH/DCM) to afford the title compound. ¹H NMR (400 MHz, MeOD) δ 7.22 (d, J=8.6 Hz, 1H), 6.58 (d, J=2.3, 1H), 6.53 (dd, J=8.3, 2.4 Hz, 1H), 3.84 (s, 3H), 3.83-3.80 (m, 5H), 3.35 (s, 1H), 1.89 (dd, J=13.7, 3.7 Hz, 2H), 1.65 (dd, J=13.6, 8.1 Hz, 2H); ES-MS [M+1]⁺: 256.2.

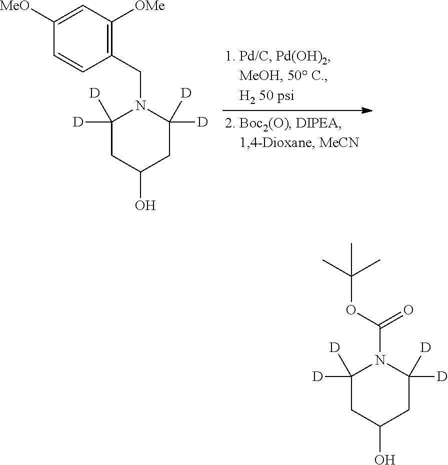 [SMILES: [2H]C1([2H])CC(O)CC([2H])([2H])N1Cc1ccc(OC)cc1OC ; [2H]C1([2H])CC(O)CC([2H])([2H])N1C(=O)OC(C)(C)C]

tert-Butyl 4-hydroxypiperidine-1-carboxylate-2,2,6,6-d4. To a degassed solution of 1-(2,4-dimethoxybenzyl)piperidin-2,2,6,6-d4-4-ol (3.5 g, 13.7 mmol) in methanol (300 mL) was added palladium hydroxide (0.29 g, 2.1 mmol) and 10% palladium on activated carbon (0.22 g, 2.1 mmol). The reaction was charged with H2 and stirred at 50° C. under 50 psi for 48 h. The reaction mixture was filtered over celite, washed with methanol, and concentrated under reduced pressure. The solid was combined with 1,4-dioxane (45 mL), acetonitrile (45 mL), and N,N-diisopropylethylamine (2.9 mL, 16.4 mmol). To the solution was added di-tert-butyl dicarbonate (4.7 mL, 20.5 mmol) and the reaction stirred at room temperature. After 3 h, the reaction was concentrated, and the crude oil was purified by normal-phase chromatography (0-20% MeOH/DCM) to afford the title compound (2.18 g). ¹H NMR (400 MHz, MeOD) δ 3.79-3.71 (m, 1H), 1.79 (dd, J=13.1, 3.8 Hz, 2H), 1.45 (s, 9H), 1.39-1.34 (m, 2H).

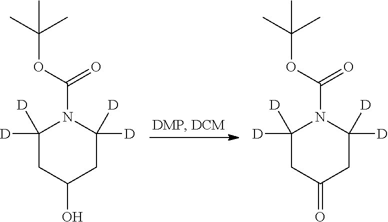 [SMILES: [2H]C1([2H])CC(O)CC([2H])([2H])N1C(=O)OC(C)(C)C ; [2H]C1([2H])CC(=O)CC([2H])([2H])N1C(=O)OC(C)(C)C]

tert-Butyl 4-oxopiperidine-1-carboxylate-2,2,6,6-d₄. To a solution of tert-butyl 4-hydroxypiperidine-1-carboxylate-2,2,6,6-d4 (2.18 g, 10.6 mmol) in DCM (30 mL) was added Dess-Martin periodinane (6.75 g, 15.9 mmol). The reaction stirred at room temperature for 18 h. The reaction was concentrated over Celite® and purified by normal-phase chromatography (0-50% EtOAc/Hexanes) to afford the title compound (1.69 g). ¹H NMR (400 MHz, CDCl₃) δ 2.42 (s, 4H), 1.49 (s, 9H).

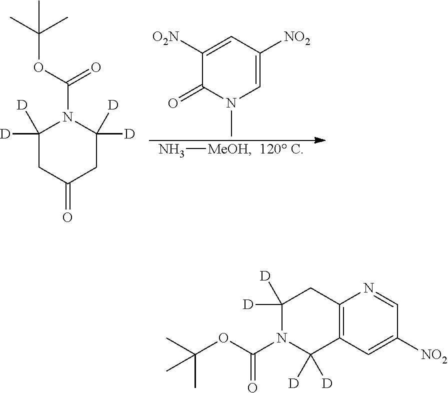

tert-Butyl 3-nitro-7,8-dihydro-1,6-naphthyridine-6(5H)-carboxylate-5,5,7,7-d₄. In four separate microwave vials were combined equal portions of 1-methyl-3,5-dinitro-2-pyridone (1.0 g, 5.1 mmol), tert-butyl 4-oxopiperidine-1-carboxylate-2,2,6,6-d4(1.0 g, 5.1 mmol), and 2M ammonia-methanol solution (20.2 mL). The mixture was heated at 120° C. for 20 min with microwave irradiation. The reaction was concentrated over Celite® and purified by normal-phase chromatography (0-30% EtOAc/Hexanes) to afford the title compound (1.23 g). ¹H NMR (400 MHz, CDCl₃) δ 9.25 (d, J=2.5 Hz, 1H), 8.23 (d, J=2.5, 1H), 3.11 (s, 2H), 1.50 (s, 9H); ES-MS [M+1]⁺: 284.1.

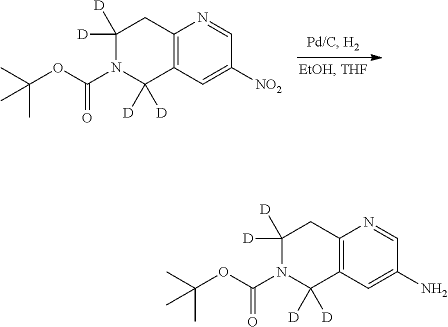 [SMILES: [2H]C1([2H])Cc2ncc([N+](=O)[O-])cc2C([2H])([2H])N1C(=O)OC(C)(C)C ; [2H]C1([2H])Cc2ncc(N)cc2C([2H])([2H])N1C(=O)OC(C)(C)C]

tert-Butyl 3-amino-7,8-dihydro-1,6-naphthyridine-6(5H)-carboxylate-5,5,7,7-d4. To a solution of tert-butyl 3-nitro-7,8-dihydro-1,6-naphthyridine-6(5H)-carboxylate-5,5,7,7-d4 (1.23 g, 4.3 mmol) in ethanol (10 mL) and THF (10 mL) was added 10% palladium on activated carbon (527 mg, 4.9 mmol). The mixture was degassed and placed under H2 balloon at 1 atm for 3 h. The reaction was filtered through Celite®, washed with EtOH, and the filtrate was concentrated to afford the title compound (1.03 g). ¹H NMR (400 MHz, CDCl₃) δ 7.94 (d, J=2.6 Hz, 1H), 6.73 (d, J=2.6, 1H), 3.60 (s, 2H), 2.87 (s, 2H), 1.48 (s, 9H); ES-MS [M+1]⁺: 254.1.

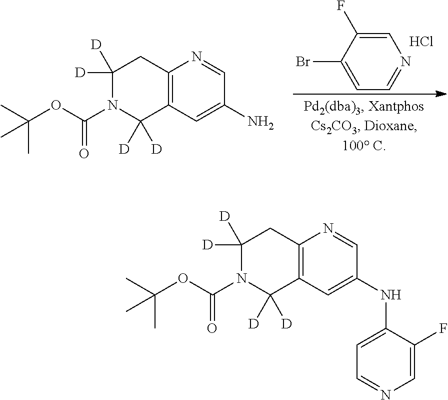 [SMILES: [2H]C1([2H])Cc2ncc(N)cc2C([2H])([2H])N1C(=O)OC(C)(C)C ; Fc1cnccc1Br ; Cl ; [2H]C1([2H])Cc2ncc(Nc3ccncc3F)cc2C([2H])([2H])N1C(=O)OC(C)(C)C]

tert-Butyl 3-((3-fluoropyridin-4-yl)amino)-7,8-dihydro-1,6-naphthyridine-6(5H)-carboxylate-5,5,7,7-d₄. In a vial were combined 4-bromo-3-fluoropyridine hydrochloride (415 mg, 1.9 mmol), tert-butyl 3-amino-7,8-dihydro-1,6-naphthyridine-6(5H)-carboxylate-5,5,7,7-d4 (330 mg, 1.3 mmol), tris(dibenzylideneacetone)dipalladium(0) (119 mg, 0.1 mmol), Xantphos (113 mg, 0.2 mmol), and cesium carbonate (1.7 g, 5.2 mmol) in 1,4-dioxane (6.5 mL). The reaction was degassed and heated at 100° C. for 2 h. The mixture was cooled, filtered through a pad of Celite®, and washed with 3:1 CHCl₃/IPA. The solvents were removed, and the crude product was purified by normal-phase chromatography (0-5% MeOH/DCM) to afford the title compound (314 mg). ¹H NMR (400 MHz, CDCl₃) δ 8.38 (d, J=2.5 Hz, 1H), 8.32 (d, J=2.9 Hz, 1H), 8.13 (d, J=5.5 Hz, 1H), 7.33 (d, J=2.5 Hz, 1H), 6.94 (dd, J=7.0, 5.8, 1H), 6.24 (s, 1H), 3.00 (s, 2H), 1.50 (s, 9H); ES-MS [M+1]⁺: 349.3.

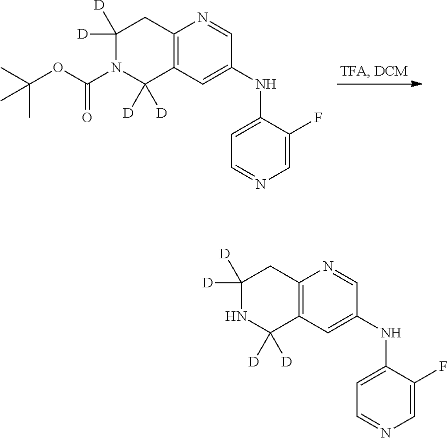 [SMILES: [2H]C1([2H])Cc2ncc(Nc3ccncc3F)cc2C([2H])([2H])N1C(=O)OC(C)(C)C ; [2H]C1([2H])Cc2ncc(Nc3ccncc3F)cc2C([2H])([2H])N1]

N-(3-Fluoropyridin-4-yl)-5,6,7,8-tetrahydro-1,6-naphthyridin-5,5,7,7-d₄-3-amine. In a vial were combined tert-butyl 3-((3-fluoropyridin-4-yl)amino)-7,8-dihydro-1,6-naphthyridine-6(5H)-carboxylate-5,5,7,7-d4 (314 mg, 0.9 mmol), trifluoroacetic acid (1.07 mL, 14.0 mmol), and DCM (4 mL). The reaction stirred at room temperature for 2 h. The reaction was concentrated and purified by SCX cartridge, eluting with 2N NH₃/MeOH solution. Solvents were removed to afford the title compound (178 mg). ¹H NMR (400 MHz, CDCl₃) δ 8.30 (d, J=2.5 Hz, 1H), 8.23 (s, 1H), 8.17 (d, J=5.7 Hz, 1H), 7.22 (d, J=2.6 Hz, 1H), 6.79 (d, J=5.6, 1H), 5.68 (s, 1H), 2.94 (s, 2H); ES-MS [M+1]⁺: 249.3.

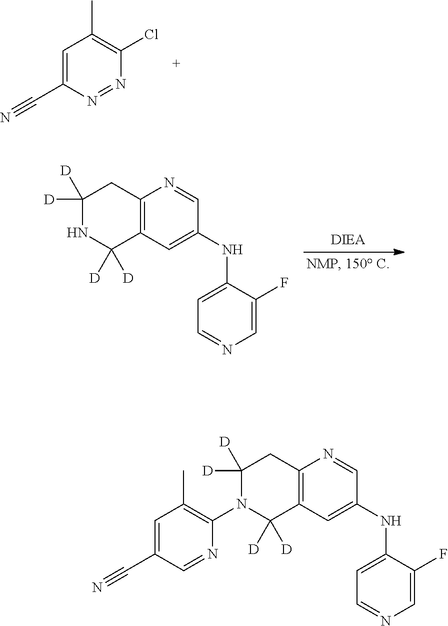

6-(3-((3-Fluoropyridin-4-yl)amino)-7,8-dihydro-1,6-naphthyridin-6(5H)-yl-5,5,7,7-d₄)-5-methylpyridazine-3-carbonitrile. To a vial was added 6-chloro-5-methylpyridazine-3-carbonitrile (12 mg, 0.08 mmol), DIEA (67 μL) and N-(3-fluoropyridin-4-yl)-5,6,7,8-tetrahydro-1,6-naphthyridin-5,5,7,7-d4-3-amine (19 mg, 0.08 mmol) in NMP (0.4 mL). The reaction was heated to 120° C. After 12 h, the reaction mixture was purified using reverse phase HPLC to afford title compound. ES-MS [M+1]⁺: 366.4.

### Example 38

### 6-(3-(3,5-dimethylisoxazol-4-yl)-7,8-dihydro-1,6-naphthyridin-6(5H)-yl-5,5,7,7-d₄)-5-methylpyridazine-3-carbonitrile

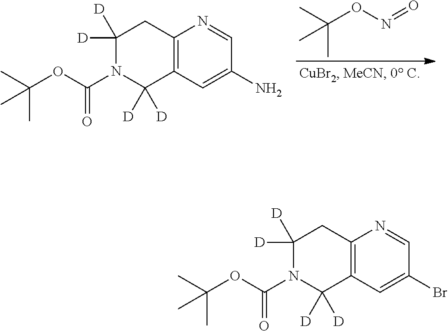

tert-Butyl 3-bromo-7,8-dihydro-1,6-naphthyridine-6(5H)-carboxylate-5,5,7,7-d₄. Cupric bromide (767 mg, 3.4 mmol) was added to a solution of tert-butyl 3-amino-7,8-dihydro-1,6-naphthyridine-6(5H)-carboxylate-5,5,7,7-d4 (580 mg, 2.3 mmol) in MeCN (8 mL). The reaction was cooled to 0° C. followed by dropwise addition of tert-butyl nitrite (0.33 mL, 2.8 mmol). The reaction stirred at 0° C. for 1 h and then room temperature for 5 h. The mixture was diluted with water and CHCl₃/IPA. The layers were separated and the aqueous layer was re-extracted with CHCl₃/IPA (2×). The combined organic phases were washed with brine (2×), dried (MgSO₄), filtered, and concentrated. The crude oil was purified by normal-phase chromatography (0-40% EtOAc/Hexanes) to afford the title compound (520 mg). ¹H NMR (400 MHz, CDCl₃) δ 8.47 (d, J=2.2 Hz, 1H), 7.56 (d, J=2.2, 1H), 2.93 (s, 2H), 1.48 (s, 9H); ES-MS [M+1]⁺: 317.1/319.1.

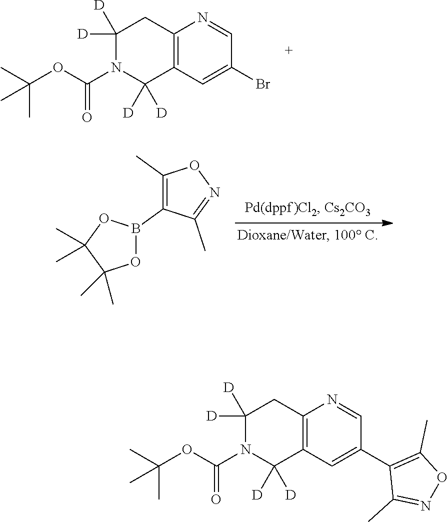

tert-Butyl 3-(3,5-dimethylisoxazol-4-yl)-7,8-dihydro-1,6-naphthyridine-6(5H)-carboxylate-5,5,7,7-d₄. tert-Butyl 3-bromo-7,8-dihydro-1,6-naphthyridine-6(5H)-carboxylate-5,5,7,7-d4 (200 mg, 0.63 mmol), 3,5-dimethylisoxazole-4-boronic acid pinacol ester (169 mg, 0.76 mmol), cesium carbonate (413 mg, 1.26 mmol), and Pd(dppf)Cl₂ (69 mg, 0.09 mmol) were combined in 1,4-dioxane (2.5 mL) and water (0.5 mL) and degassed (3×). The reaction was heated at 100° C. for 2.5 h. The mixture was filtered through a pad of Celite®, washed with EtOAc/DCM, and the filtrate was concentrated under reduced pressure. The oil was purified by normal-phase chromatography (0-4% MeOH/DCM) to afford the title compound (202 mg). ¹H NMR (400 MHz, CDCl₃) δ 8.34 (d, J=2.0 Hz, 1H), 7.31 (d, J=2.1 Hz, 1H), 3.04 (s, 2H), 2.41 (s, 3H), 2.27 (s, 3H), 1.51 (s, 9H); ES-MS [M+1]⁺: 334.1.

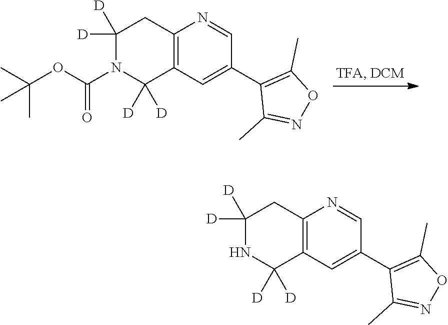 [SMILES: [2H]C1([2H])Cc2ncc(-c3c(C)noc3C)cc2C([2H])([2H])N1C(=O)OC(C)(C)C ; [2H]C1([2H])Cc2ncc(-c3c(C)noc3C)cc2C([2H])([2H])N1]

3,5-Dimethyl-4-(5,6,7,8-tetrahydro-1,6-naphthyridin-3-yl-5,5,7,7-d)isoxazole. tert-Butyl 3-(3,5-dimethylisoxazol-4-yl)-7,8-dihydro-1,6-naphthyridine-6(5H)-carboxylate-5,5,7,7-d4 (200.mg, 0.6 mmol) was combined with DCM (3 mL) and trifluoroacetic acid (0.69 mL, 9.0 mmol). After 2 h, the reaction was complete and was concentrated in vacuo. The crude oil was purified by SCX Cartridge, washing with MeOH and eluting compound with 7N NH₃/MeOH solution. Solvents were removed to afford the title compound (114 mg). ¹H NMR (400 MHz, CDCl₃) δ 8.31 (d, J=2.2 Hz, 1H), 7.20 (d, J=2.2 Hz, 1H), 2.98 (s, 2H), 2.39 (s, 3H), 2.25 (s, 3H); ES-MS [M+1]⁺: 234.3.

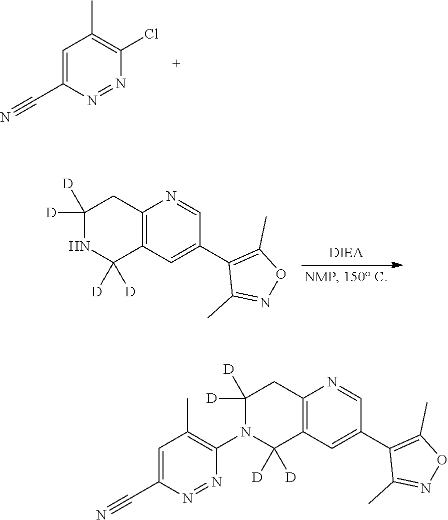

6-(3-(3,5-Dimethylisoxazol-4-yl)-7,8-dihydro-1,6-naphthyridin-6(5H)-yl-5,5,7,7-d₄)-5-methylpyridazine-3-carbonitrile. The title compound was prepared in a similar manner as 6-(3-((3-fluoropyridin-4-yl)amino)-7,8-dihydro-1,6-naphthyridin-6(5N)-yl-5,5,7,7-d₄)-5-methylpyridazine-3-carbonitrile (Example 37). ES-MS [M+1]⁺: 351.0.

The compounds shown in Table 1 were prepared using the methods shown in the preceding Schemes and Examples with the appropriate starting materials.

TABLE 1

| Cpd. | | | ES-MS |
|---|---|---|---|
| No. | Name | Structure | [M + 1]⁺ |
| 1 | 5-methyl-6-(3-pyrrolidin-1-yl-7,8- dihydro-5H-1,6-naphthyridin-6- yl)pyridine-3-carbonitrile | 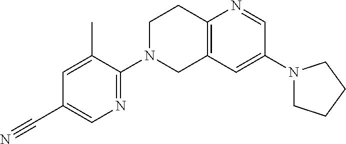 [SMILES: Cc1cc(C#N)cnc1N1CCc2ncc(N3CCCC3)cc2C1] | 320.2 |
| 2 | 6-[3-(4-fluoroanilino)-7,8-dihydro- 5H-1,6-naphthyridin-6-yl]-5- methyl-pyridine-3-carbonitrile | 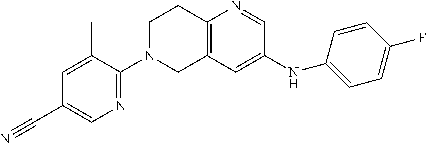 [SMILES: Cc1cc(C#N)cnc1N1CCc2ncc(Nc3ccc(F)cc3)cc2C1] | 360.2 |
| 3 | 6-[3-(4-fluorophenyl)-7,8-dihydro- 5H-1,6-naphthyridin-6-yl]-5- methyl-pyridine-3-carbonitrile | 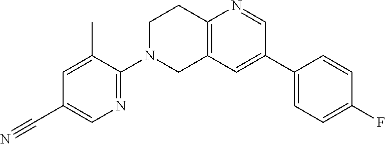 [SMILES: Cc1cc(C#N)cnc1N1CCc2ncc(-c3ccc(F)cc3)cc2C1] | 345.2 |
| 4 | 6-[3-[(4- fluorophenyl)methylamino]-7,8- dihydro-5H-1,6-naphthyridin-6-yl]- 5-methyl-pyridine-3-carbonitrile | 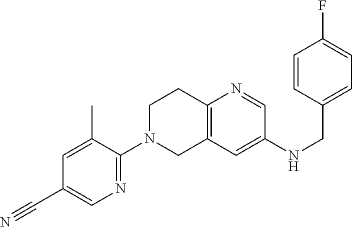 [SMILES: Cc1cc(C#N)cnc1N1CCc2ncc(NCc3ccc(F)cc3)cc2C1] | 374.2 |
| 5 | 6-[3-[(3-fluoro-4-methoxy- phenyl)methylamino]-7,8-dihydro- 5H-1,6-naphthyridin-6-yl]-5- methyl-pyridine-3-carbonitrile | 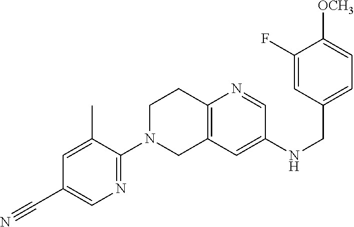 [SMILES: COc1ccc(CNc2cnc3c(c2)CN(c2ncc(C#N)cc2C)CC3)cc1F] | 404.2 |
| 6 | 5-methyl-6-[3-(2-pyridylamino)- 7,8-dihydro-5H-1,6-naphthyridin-6- yl]pyridine-3-carbonitrile | 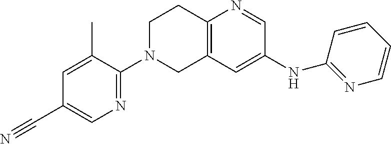 [SMILES: Cc1cc(C#N)cnc1N1CCc2ncc(Nc3ccccn3)cc2C1] | 343.2 |
| 7 | 6-[3-(2-fluoroanilino)-7,8-dihydro- 5H-1,6-naphthyridin-6-yl]-5- methyl-pyridine-3-carbonitrile | 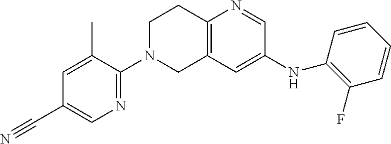 [SMILES: Cc1cc(C#N)cnc1N1CCc2ncc(Nc3ccccc3F)cc2C1] | 360.2 |
| 8 | 6-[3-(1,4-dioxan-2- ylmethylamino)-7,8-dihydro-5H- 1,6-naphthyridin-6-yl]-5-methyl- pyridine-3-carbonitrile | 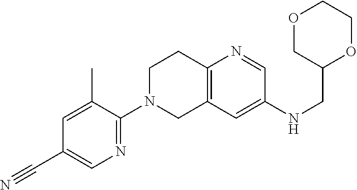 [SMILES: Cc1cc(C#N)cnc1N1CCc2ncc(NCC3COCCO3)cc2C1] | 366.2 |
| 9 | 5-methyl-6-[3-(tetrahydrofuran-2- ylmethylamino)-7,8-dihydro-5H- 1,6-naphthyridin-6-yl]pyridine-3- carbonitrile | 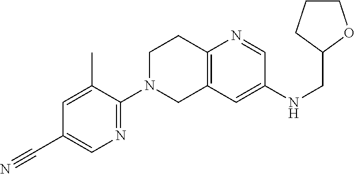 [SMILES: Cc1cc(C#N)cnc1N1CCc2ncc(NCC3CCCO3)cc2C1] | 350.2 |
| 10 | 6-[3-[1- (cyclopropylmethyl)pyrazol-3-yl]- 7,8-dihydro-5H-1,6-naphthyridin-6- yl]-4,5-dimethyl-pyridazine-3- carbonitrile | 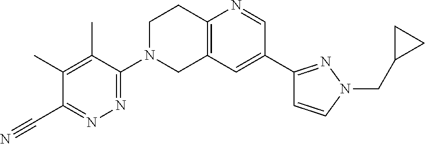 [SMILES: Cc1c(C#N)nnc(N2CCc3ncc(-c4ccn(CC5CC5)n4)cc3C2)c1C] | 386.2 |
| 11 | 4,5-dimethyl-6-[3-(1- methylpyrazol-4-yl)-7,8-dihydro- 5H-1,6-naphthyridin-6- yl]pyridazine-3-carbonitrile | 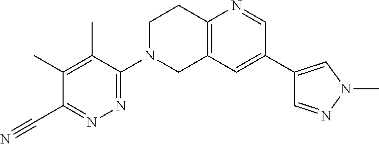 [SMILES: Cc1c(C#N)nnc(N2CCc3ncc(-c4cnn(C)c4)cc3C2)c1C] | 346.2 |
| 12 | 4,5-dimethyl-6-[3-(2-methyl-4- pyridyl)-7,8-dihydro-5H-1,6- naphthyridin-6-yl]pyridazine-3- carbonitrile | 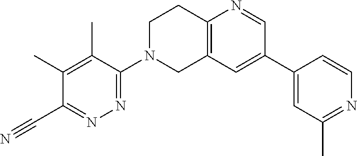 [SMILES: Cc1cc(-c2cnc3c(c2)CN(c2nnc(C#N)c(C)c2C)CC3)ccn1] | 357.2 |
| 13 | 6-[3-(1-cyclopropylpyrazol-4-yl)- 7,8-dihydro-5H-1,6-naphthyridin-6- yl]-4,5-dimethyl-pyridazine-3- carbonitrile | 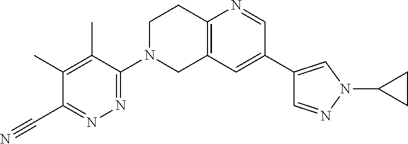 [SMILES: Cc1c(C#N)nnc(N2CCc3ncc(-c4cnn(C5CC5)c4)cc3C2)c1C] | 372.2 |
| 14 | 4,5-dimethyl-6-[3-(2- methylpyrazol-3-yl)-7,8-dihydro- 5H-1,6-naphthyridin-6- yl]pyridazine-3-carbonitrile | 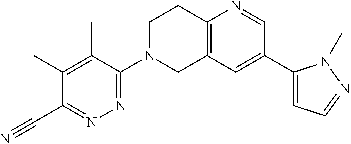 [SMILES: Cc1c(C#N)nnc(N2CCc3ncc(-c4ccnn4C)cc3C2)c1C] | 346.2 |
| 15 | 6-[3-(1-cyclohexylpyrazol-3-yl)- 7,8-dihydro-5H-1,6-naphthyridin-6- yl]-4,5-dimethyl-pyridazine-3- carbonitrile | 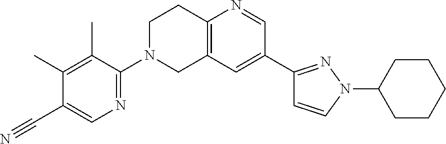 [SMILES: Cc1c(C#N)cnc(N2CCc3ncc(-c4ccn(C5CCCCC5)n4)cc3C2)c1C] | 414.2 |
| 16 | 4,5-dimethyl-6-[3-(1- propylpyrazol-4-yl)-7,8-dihydro- 5H-1,6-naphthyridin-6- yl]pyridazine-3-carbonitrile | 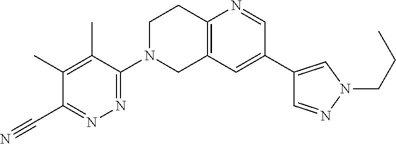 [SMILES: CCCn1cc(-c2cnc3c(c2)CN(c2nnc(C#N)c(C)c2C)CC3)cn1] | 374.2 |
| 17 | 4,5-dimethyl-6-[3-(3- methylbenzimidazol-5-yl)-7,8- dihydro-5H-1,6-naphthyridin-6- yl]pyridazine-3-carbonitrile | 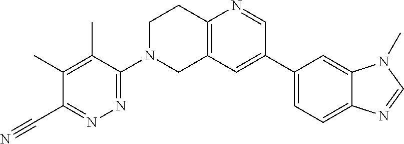 [SMILES: Cc1c(C#N)nnc(N2CCc3ncc(-c4ccc5ncn(C)c5c4)cc3C2)c1C] | 396.2 |
| 18 | 6-[3-(2-fluorophenyl)-7,8-dihydro- 5H-1,6-naphthyridin-6-yl]-4,5- dimethyl-pyridazine-3-carbonitrile | 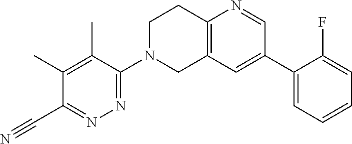 [SMILES: Cc1c(C#N)nnc(N2CCc3ncc(-c4ccccc4F)cc3C2)c1C] | 360.2 |
| 19 | 6-(3-imidazo[1,2-a]pyridin-6-yl- 7,8-dihydro-5H-1,6-naphthyridin-6- yl)-4,5-dimethyl-pyridazine-3- carbonitrile | 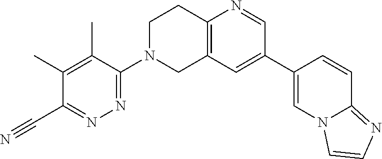 [SMILES: Cc1c(C#N)nnc(N2CCc3ncc(-c4ccc5nccn5c4)cc3C2)c1C] | 382.2 |
| 20 | 4,5-dimethyl-6-[3-(2-phenylethyl)- 7,8-dihydro-5H-1,6-naphthyridin-6- yl]pyridazine-3-carbonitrile | 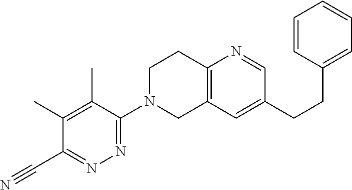 [SMILES: Cc1c(C#N)nnc(N2CCc3ncc(CCc4ccccc4)cc3C2)c1C] | 370.2 |
| 21 | 6-[3-(3-fluorophenyl)-7,8-dihydro- 5H-1,6-naphthyridin-6-yl]-4,5- dimethyl-pyridazine-3-carbonitrile | 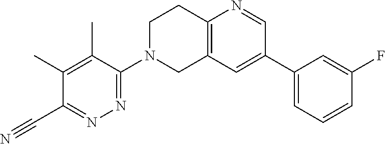 [SMILES: Cc1c(C#N)nnc(N2CCc3ncc(-c4cccc(F)c4)cc3C2)c1C] | 360.2 |
| 22 | 4,5-dimethyl-6-(3-pyrrolidin-1-yl- 7,8-dihydro-5H-1,6-naphthyridin-6- yl)pyridazine-3-carbonitrile | 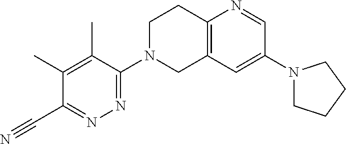 [SMILES: Cc1c(C#N)nnc(N2CCc3ncc(N4CCCC4)cc3C2)c1C] | 335.2 |
| 23 | 4,5-dimethyl-6-[3-(1-piperidyl)- 7,8-dihydro-5H-1,6-naphthyridin-6- yl]pyridazine-3-carbonitrile | 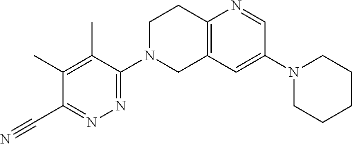 [SMILES: Cc1c(C#N)nnc(N2CCc3ncc(N4CCCCC4)cc3C2)c1C] | 349.3 |
| 24 | 6-[3-[(3- fluorophenyl)methylamino]-7,8- dihydro-5H-1,6-naphthyridin-6-yl]- 4,5-dimethyl-pyridazine-3- carbonitrile | 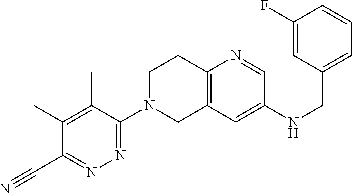 [SMILES: Cc1c(C#N)nnc(N2CCc3ncc(NCc4cccc(F)c4)cc3C2)c1C] | 389.2 |
| 25 | 6-[3-[(4- methoxyphenyl)methylamino]-7,8- dihydro-5H-1,6-naphthyridin-6-yl]- 4,5-dimethyl-pyridazine-3- carbonitrile | 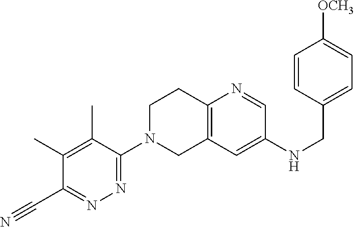 [SMILES: COc1ccc(CNc2cnc3c(c2)CN(c2nnc(C#N)c(C)c2C)CC3)cc1] | 401.2 |
| 26 | 6-[3-[(4- cyanophenyl)methylamino]-7,8- dihydro-5H-1,6-naphthyridin-6-yl]- 4,5-dimethyl-pyridazine-3- carbonitrile | 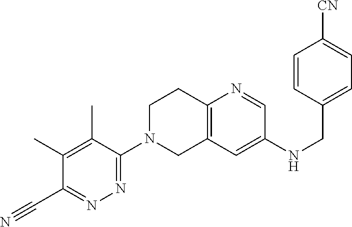 [SMILES: Cc1c(C#N)nnc(N2CCc3ncc(NCc4ccc(C#N)cc4)cc3C2)c1C] | 396.2 |
| 27 | 6-[3-[(3- methoxyphenyl)methylamino]-7,8- dihydro-5H-1,6-naphthyridin-6-yl]- 4,5-dimethyl-pyridazine-3- carbonitrile | 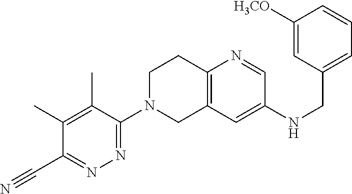 [SMILES: COc1cccc(CNc2cnc3c(c2)CN(c2nnc(C#N)c(C)c2C)CC3)c1] | 401.2 |
| 28 | 6-[3-(3,3-dimethylazetidin-1-yl)- 7,8-dihydro-5H-1,6-naphthyridin-6- yl]-4,5-dimethyl-pyridazine-3- carbonitrile | 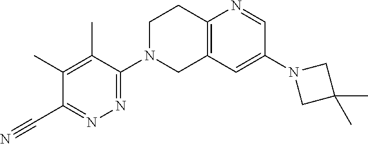 [SMILES: Cc1c(C#N)nnc(N2CCc3ncc(N4CC(C)(C)C4)cc3C2)c1C] | 349.2 |
| 29 | 4,5-dimethyl-6-[3-[(6-methyl-3- pyridyl)methylamino]-7,8-dihydro- 5H-1,6-naphthyridin-6- yl]pyridazine-3-carbonitrile | 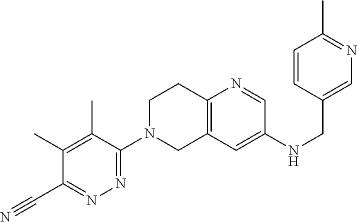 [SMILES: Cc1ccc(CNc2cnc3c(c2)CN(c2nnc(C#N)c(C)c2C)CC3)cn1] | 386.2 |
| 30 | 6-[3-(2-fluoroanilino)-7,8-dihydro- 5H-1,6-naphthyridin-6-yl]-4,5- dimethyl-pyridazine-3-carbonitrile | 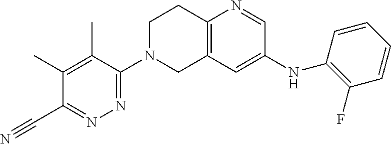 [SMILES: Cc1c(C#N)nnc(N2CCc3ncc(Nc4ccccc4F)cc3C2)c1C] | 375.2 |
| 31 | 4,5-dimethyl-6-(3-thiazol-5-yl-7,8- dihydro-5H-1,6-naphthyridin-6- yl)pyridazine-3-carbonitrile | 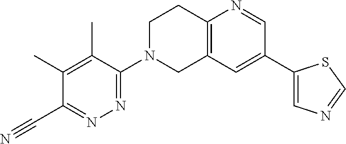 [SMILES: Cc1c(C#N)nnc(N2CCc3ncc(-c4cncs4)cc3C2)c1C] | 349.2 |
| 32 | 6-[3-(5-cyano-2-fluoro-anilino)- 7,8-dihydro-5H-1,6-naphthyridin-6- yl]-4,5-dimethyl-pyridazine-3- carbonitrile | 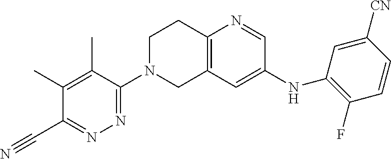 [SMILES: Cc1c(C#N)nnc(N2CCc3ncc(Nc4cc(C#N)ccc4F)cc3C2)c1C] | 400.2 |
| 33 | 6-[3-(2-chloro-6-fluoro-anilino)- 7,8-dihydro-5H-1,6-naphthyridin-6- yl]-4,5-dimethyl-pyridazine-3- carbonitrile | 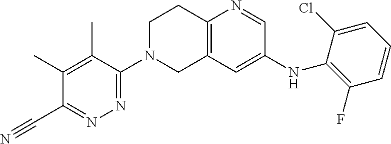 [SMILES: Cc1c(C#N)nnc(N2CCc3ncc(Nc4c(F)cccc4Cl)cc3C2)c1C] | 409.2 |
| 34 | 6-[3-(2-fluoro-3-methoxy-anilino)- 7,8-dihydro-5H-1,6-naphthyridin-6- yl]-4,5-dimethyl-pyridazine-3- carbonitrile | 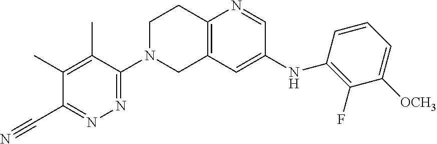 [SMILES: COc1cccc(Nc2cnc3c(c2)CN(c2nnc(C#N)c(C)c2C)CC3)c1F] | 405.2 |
| 35 | 6-[3-[2-fluoro-3- (trifluoromethyl)anilino]-7,8- dihydro-5H-1,6-naphthyridin-6-yl]- 4,5-dimethyl-pyridazine-3- carbonitrile | 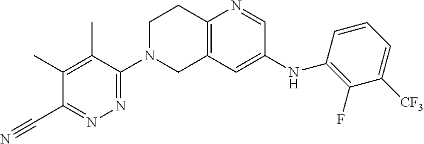 [SMILES: Cc1c(C#N)nnc(N2CCc3ncc(Nc4cccc(C(F)(F)F)c4F)cc3C2)c1C] | 443.2 |
| 36 | 6-[3-(3-chloro-2-fluoro-anilino)- 7,8-dihydro-5H-1,6-naphthyridin-6- yl]-4,5-dimethyl-pyridazine-3- carbonitrile | 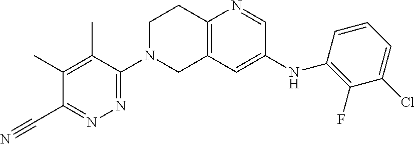 [SMILES: Cc1c(C#N)nnc(N2CCc3ncc(Nc4cccc(Cl)c4F)cc3C2)c1C] | 409.2 |
| 37 | 6-[3-(2-fluoro-5-methyl-anilino)- 7,8-dihydro-5H-1,6-naphthyridin-6- yl]-4,5-dimethyl-pyridazine-3- carbonitrile | 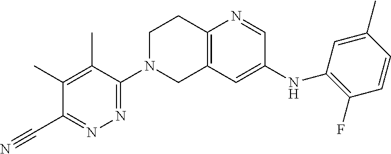 [SMILES: Cc1ccc(F)c(Nc2cnc3c(c2)CN(c2nnc(C#N)c(C)c2C)CC3)c1] | 389.2 |
| 38 | 6-[3-(2,4-difluoroanilino)-7,8- dihydro-5H-1,6-naphthyridin-6-yl]- 4,5-dimethyl-pyridazine-3- carbonitrile | 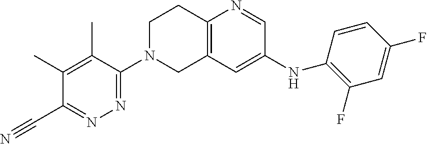 [SMILES: Cc1c(C#N)nnc(N2CCc3ncc(Nc4ccc(F)cc4F)cc3C2)c1C] | 393.2 |
| 39 | 6-[3-(2-chloroanilino)-7,8-dihydro- 5H-1,6-naphthyridin-6-yl]-4,5- dimethyl-pyridazine-3-carbonitrile | 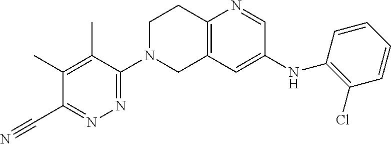 [SMILES: Cc1c(C#N)nnc(N2CCc3ncc(Nc4ccccc4Cl)cc3C2)c1C] | 391.2 |
| 40 | 6-[3-[2-(dimethylamino)anilino]- 7,8-dihydro-5H-1,6-naphthyridin-6- yl]-4,5-dimethyl-pyridazine-3- carbonitrile | 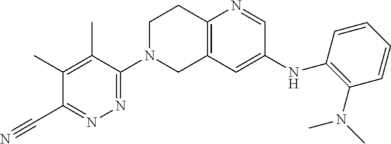 [SMILES: Cc1c(C#N)nnc(N2CCc3ncc(Nc4ccccc4N(C)C)cc3C2)c1C] | 400.2 |
| 41 | 6-[3-(2-cyanoanilino)-7,8-dihydro- 5H-1,6-naphthyridin-6-yl]-4,5- dimethyl-pyridazine-3-carbonitrile | 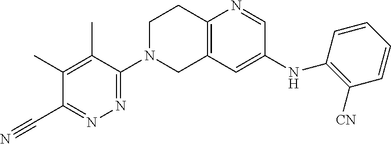 [SMILES: Cc1c(C#N)nnc(N2CCc3ncc(Nc4ccccc4C#N)cc3C2)c1C] | 382.2 |
| 42 | 4,5-dimethyl-6-[3-(2- methylanilino)-7,8-dihydro-5H-1,6- naphthyridin-6-yl]pyridazine-3- carbonitrile | 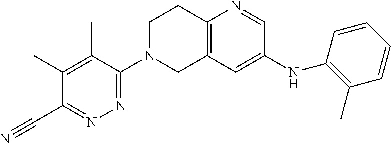 [SMILES: Cc1ccccc1Nc1cnc2c(c1)CN(c1nnc(C#N)c(C)c1C)CC2] | 371.2 |
| 43 | 4,5-dimethyl-6-[3-[2- (trifluoromethyl)anilino]-7,8- dihydro-5H-1,6-naphthyridin-6- yl]pyridazine-3-carbonitrile | 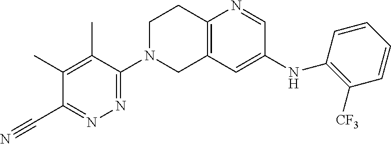 [SMILES: Cc1c(C#N)nnc(N2CCc3ncc(Nc4ccccc4C(F)(F)F)cc3C2)c1C] | 425.2 |
| 44 | 4,5-dimethyl-6-[3-[2- (trifluoromethoxy)anilino]-7,8- dihydro-5H-1,6-naphthyridin-6- yl]pyridazine-3-carbonitrile | 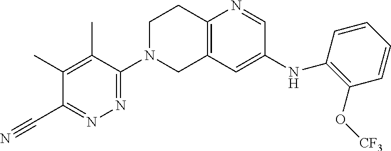 [SMILES: Cc1c(C#N)nnc(N2CCc3ncc(Nc4ccccc4OC(F)(F)F)cc3C2)c1C] | 441.2 |
| 45 | 6-(7,8-dihydro-5H-1,6- naphthyridin-6-yl)-5-methyl- pyridine-3-carbonitrile | 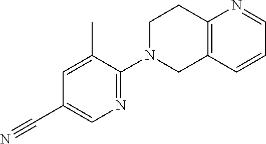 [SMILES: Cc1cc(C#N)cnc1N1CCc2ncccc2C1] | 251.2 |
| 46 | 6-[3-(4-chloro-2-fluoro-anilino)- 7,8-dihydro-5H-1,6-naphthyridin-6- yl]-4,5-dimethyl-pyridazine-3- carbonitrile | 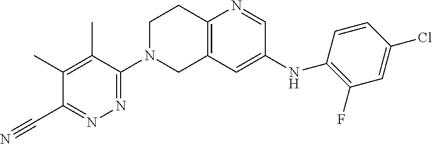 [SMILES: Cc1c(C#N)nnc(N2CCc3ncc(Nc4ccc(Cl)cc4F)cc3C2)c1C] | 409.2 |
| 47 | 4,5-dimethyl-6-[3-(3-pyridyl)-7,8- dihydro-5H-1,6-naphthyridin-6- yl]pyridazine-3-carbonitrile | 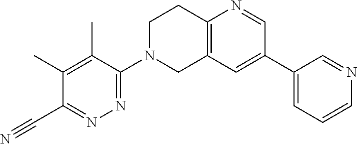 [SMILES: Cc1c(C#N)nnc(N2CCc3ncc(-c4cccnc4)cc3C2)c1C] | 343.2 |
| 48 | 4,5-dimethyl-6-(3-pyridazin-4-yl- 7,8-dihydro-5H-1,6-naphthyridin-6- yl)pyridazine-3-carbonitrile | 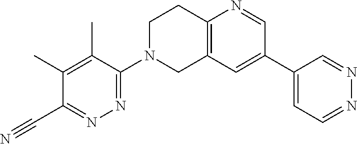 [SMILES: Cc1c(C#N)nnc(N2CCc3ncc(-c4ccnnc4)cc3C2)c1C] | 344.2 |
| 49 | 4,5-dimethyl-6-(3-pyrimidin-5-yl- 7,8-dihydro-5H-1,6-naphthyridin-6- yl)pyridazine-3-carbonitrile | 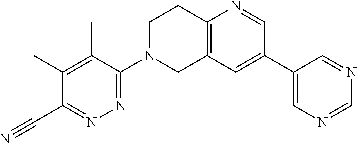 [SMILES: Cc1c(C#N)nnc(N2CCc3ncc(-c4cncnc4)cc3C2)c1C] | 344.2 |
| 50 | 4,5-dimethyl-6-[3-[2- (trifluoromethyl)-3-pyridyl]-7,8- dihydro-5H-1,6-naphthyridin-6- yl]pyridazine-3-carbonitrile | 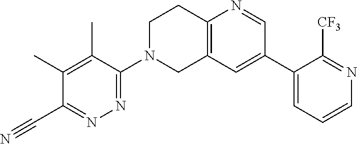 [SMILES: Cc1c(C#N)nnc(N2CCc3ncc(-c4cccnc4C(F)(F)F)cc3C2)c1C] | 411.2 |
| 51 | 6-[3-(6-chloro-5-fluoro-3-pyridyl)- 7,8-dihydro-5H-1,6-naphthyridin-6- yl]-4,5-dimethyl-pyridazine-3- carbonitrile | 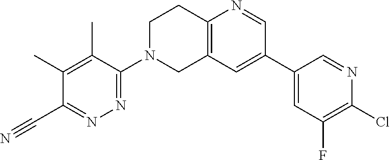 [SMILES: Cc1c(C#N)nnc(N2CCc3ncc(-c4cnc(Cl)c(F)c4)cc3C2)c1C] | 395.2 |
| 52 | 6-[3-(2-chloro-5-methoxy-3- pyridyl)-7,8-dihydro-5H-1,6- naphthyridin-6-yl]-4,5-dimethyl- pyridazine-3-carbonitrile | 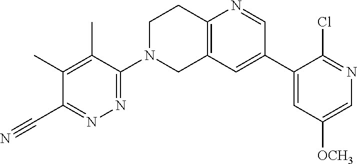 [SMILES: COc1cnc(Cl)c(-c2cnc3c(c2)CN(c2nnc(C#N)c(C)c2C)CC3)c1] | 407.2 |
| 53 | 6-[3-(2-chloro-5-fluoro-3-pyridyl)- 7,8-dihydro-5H-1,6-naphthyridin-6- yl]-4,5-dimethyl-pyridazine-3- carbonitrile | 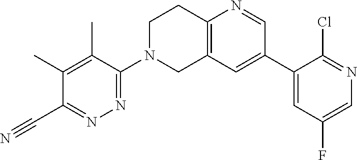 [SMILES: Cc1c(C#N)nnc(N2CCc3ncc(-c4cc(F)cnc4Cl)cc3C2)c1C] | 395.2 |
| 54 | 6-[3-(2-fluoro-3-pyridyl)-7,8- dihydro-5H-1,6-naphthyridin-6-yl]- 4,5-dimethyl-pyridazine-3- carbonitrile | 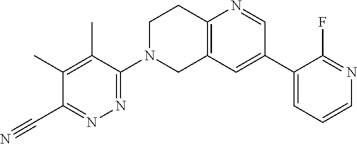 [SMILES: Cc1c(C#N)nnc(N2CCc3ncc(-c4cccnc4F)cc3C2)c1C] | 361.2 |
| 55 | 4,5-dimethyl-6-[3-(4-methyl-3- pyridyl)-7,8-dihydro-5H-1,6- naphthyridin-6-yl]pyridazine-3- carbonitrile | 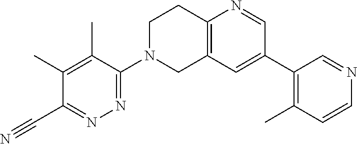 [SMILES: Cc1ccncc1-c1cnc2c(c1)CN(c1nnc(C#N)c(C)c1C)CC2] | 357.2 |
| 56 | 6-[3-(6-chloro-3-pyridyl)-7,8- dihydro-5H-1,6-naphthyridin-6-yl]- 4,5-dimethyl-pyridazine-3- carbonitrile | 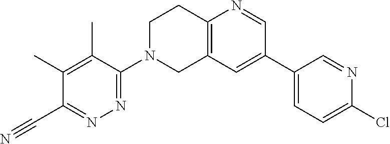 [SMILES: Cc1c(C#N)nnc(N2CCc3ncc(-c4ccc(Cl)nc4)cc3C2)c1C] | 377.2 |
| 57 | 6-(7,8-dihydro-5H-1,6- naphthyridin-6-yl)-N,5-dimethyl- pyridine-3-carboxamide | 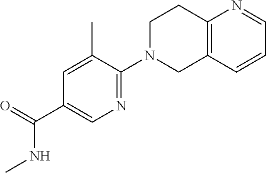 [SMILES: CNC(=O)c1cnc(N2CCc3ncccc3C2)c(C)c1] | 283.4 |
| 58 | 6-(7,8-dihydro-5H-1,6- naphthyridin-6-yl)-N-isopropyl-5- methyl-pyridine-3-carboxamide | 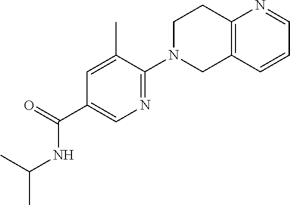 [SMILES: Cc1cc(C(=O)NC(C)C)cnc1N1CCc2ncccc2C1] | 311.4 |
| 59 | 6-(7,8-dihydro-5H-1,6- naphthyridin-6-yl)-5-methyl-N-(2- pyridylmethyl)pyridine-3- carboxamide | 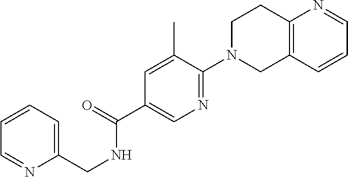 [SMILES: Cc1cc(C(=O)NCc2ccccn2)cnc1N1CCc2ncccc2C1] | 360.4 |
| 60 | 6-(7,8-dihydro-5H-1,6- naphthyridin-6-yl)-5-methyl-N-(3- pyridylmethyl)pyridine-3- carboxamide | 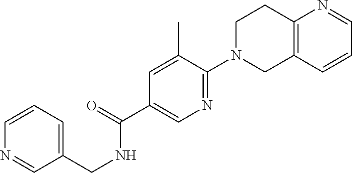 [SMILES: Cc1cc(C(=O)NCc2cccnc2)cnc1N1CCc2ncccc2C1] | 360.4 |
| 61 | 6-(7,8-dihydro-5H-1,6- naphthyridin-6-yl)-5-methyl-N-(4- pyridylmethyl)pyridine-3- carboxamide | 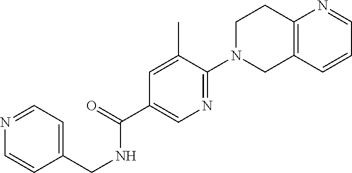 [SMILES: Cc1cc(C(=O)NCc2ccncc2)cnc1N1CCc2ncccc2C1] | 360.4 |
| 62 | 6-(7,8-dihydro-5H-1,6- naphthyridin-6-yl)-5-methyl-N- (thiazol-5-ylmethyl)pyridine-3- carboxamide | 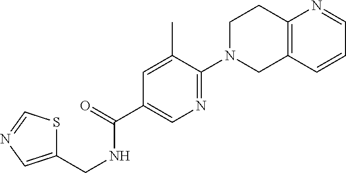 [SMILES: Cc1cc(C(=O)NCc2cncs2)cnc1N1CCc2ncccc2C1] | 366.2 |
| 63 | 6-(7,8-dihydro-5H-1,6- naphthyridin-6-yl)-5-methyl-N-[(2- methylpyrazol-3- yl)methyl]pyridine-3-carboxamide | 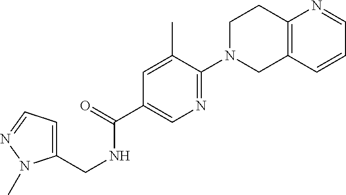 [SMILES: Cc1cc(C(=O)NCc2ccnn2C)cnc1N1CCc2ncccc2C1] | 363.2 |
| 64 | N-cyclopropyl-6-(7,8-dihydro-5H- 1,6-naphthyridin-6-yl)-5-methyl- pyridine-3-carboxamide | 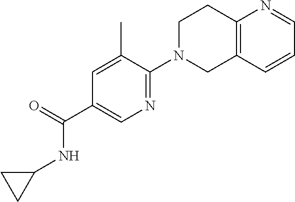 [SMILES: Cc1cc(C(=O)NC2CC2)cnc1N1CCc2ncccc2C1] | 309.2 |
| 65 | N-cyclopentyl-6-(7,8-dihydro-5H- 1,6-naphthyridin-6-yl)-5-methyl- pyridine-3-carboxamide | 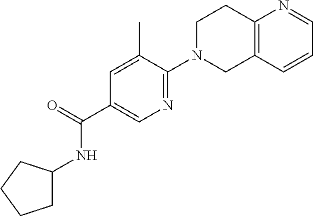 [SMILES: Cc1cc(C(=O)NC2CCCC2)cnc1N1CCc2ncccc2C1] | 337.2 |
| 66 | N-cyclohexyl-6-(7,8-dihydro-5H- 1,6-naphthyridin-6-yl)-5-methyl- pyridine-3-carboxamide | 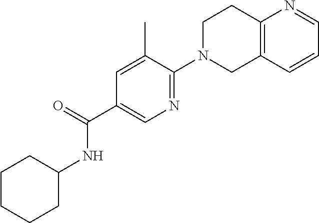 [SMILES: Cc1cc(C(=O)NC2CCCCC2)cnc1N1CCc2ncccc2C1] | 351.2 |
| 67 | 6-(7,8-dihydro-5H-1,6- naphthyridin-6-yl)-5-methyl-N-[(4- pyrazol-1- ylphenyl)methyl]pyridine-3- carboxamide | 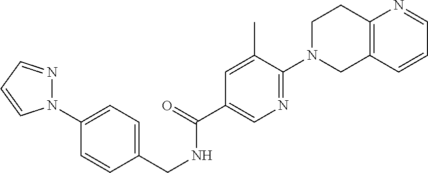 [SMILES: Cc1cc(C(=O)NCc2ccc(-n3cccn3)cc2)cnc1N1CCc2ncccc2C1] | 425.2 |
| 68 | 6-(7,8-dihydro-5H-1,6- naphthyridin-6-yl)-5-methyl-N-(6- quinolylmethyl)pyridine-3- carboxamide | 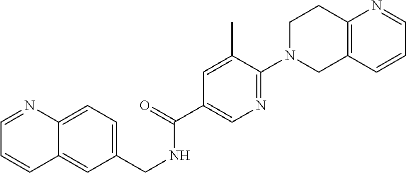 [SMILES: Cc1cc(C(=O)NCc2ccc3ncccc3c2)cnc1N1CCc2ncccc2C1] | 410.2 |
| 69 | 6-(7,8-dihydro-5H-1,6- naphthyridin-6-yl)-5-methyl-N- (pyrazolo[1,5-a]pyridin-3- ylmethyl)pyridine-3-carboxamide | 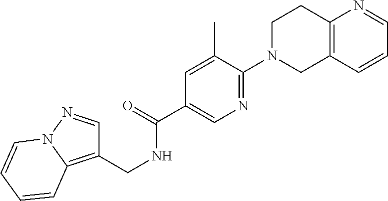 [SMILES: Cc1cc(C(=O)NCc2cnn3ccccc23)cnc1N1CCc2ncccc2C1] | 399.2 |
| 70 | N-benzyl-6-(7,8-dihydro-5H-1,6- naphthyridin-6-yl)-5-methyl- pyridine-3-carboxamide | 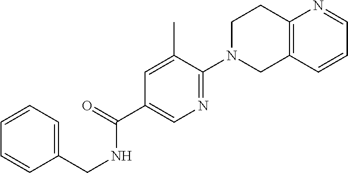 [SMILES: Cc1cc(C(=O)NCc2ccccc2)cnc1N1CCc2ncccc2C1] | 359.2 |
| 71 | methyl 6-(7,8-dihydro-5H-1,6- naphthyridin-6-yl)-5-methyl- pyridine-3-carboxylate | 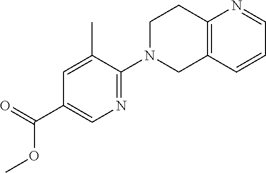 [SMILES: COC(=O)c1cnc(N2CCc3ncccc3C2)c(C)c1] | 284.2 |
| 72 | 6-[3-(2-fluoro-5-methyl-4-pyridyl)- 7,8-dihydro-5H-1,6-naphthyridin-6- yl]-4,5-dimethyl-pyridazine-3- carbonitrile | 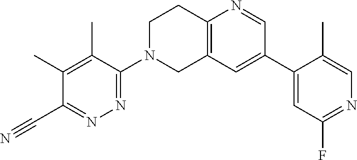 [SMILES: Cc1cnc(F)cc1-c1cnc2c(c1)CN(c1nnc(C#N)c(C)c1C)CC2] | 375.2 |
| 73 | 6-[3-(2-chloro-3-fluoro-4-pyridyl)- 7,8-dihydro-5H-1,6-naphthyridin-6- yl]-4,5-dimethyl-pyridazine-3- carbonitrile | 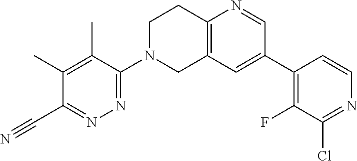 [SMILES: Cc1c(C#N)nnc(N2CCc3ncc(-c4ccnc(Cl)c4F)cc3C2)c1C] | 395 |
| 74 | 4,5-dimethyl-6-[3-(4-pyridyl)-7,8- dihydro-5H-1,6-naphthyridin-6- yl]pyridazine-3-carbonitrile | 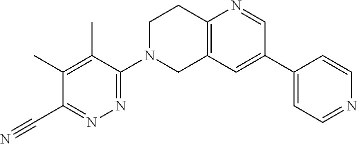 [SMILES: Cc1c(C#N)nnc(N2CCc3ncc(-c4ccncc4)cc3C2)c1C] | 343.2 |
| 75 | 6-[3-(2,6-difluoro-4-pyridyl)-7,8- dihydro-5H-1,6-naphthyridin-6-yl]- 4,5-dimethyl-pyridazine-3- carbonitrile | 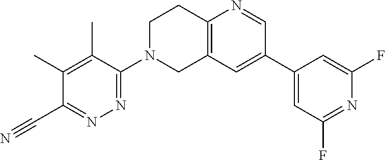 [SMILES: Cc1c(C#N)nnc(N2CCc3ncc(-c4cc(F)nc(F)c4)cc3C2)c1C] | 379.2 |
| 76 | 6-[3-(2-methoxy-4-pyridyl)-7,8- dihydro-5H-1,6-naphthyridin-6-yl]- 4,5-dimethyl-pyridazine-3- carbonitrile | 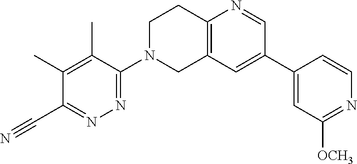 [SMILES: COc1cc(-c2cnc3c(c2)CN(c2nnc(C#N)c(C)c2C)CC3)ccn1] | 373.2 |
| 77 | 4,5-dimethyl-6-[3-(3-methyl-4- pyridyl)-7,8-dihydro-5H-1,6- naphthyridin-6-yl]pyridazine-3- carbonitrile | 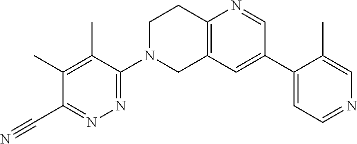 [SMILES: Cc1cnccc1-c1cnc2c(c1)CN(c1nnc(C#N)c(C)c1C)CC2] | 357.2 |
| 78 | 6-[3-[(3,5-difluoro-2- pyridyl)amino]-7,8-dihydro-5H- 1,6-naphthyridin-6-yl]-4,5- dimethyl-pyridazine-3-carbonitrile | 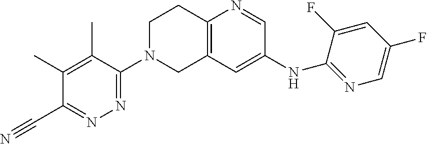 [SMILES: Cc1c(C#N)nnc(N2CCc3ncc(Nc4ncc(F)cc4F)cc3C2)c1C] | 394.2 |
| 79 | 6-[3-[(2-fluoro-6-methyl-3- pyridyl)amino]-7,8-dihydro-5H- 1,6-naphthyridin-6-yl]-4,5- dimethyl-pyridazine-3-carbonitrile | 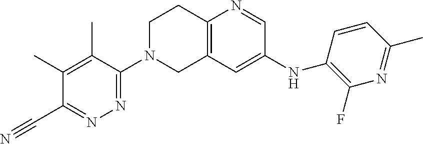 [SMILES: Cc1ccc(Nc2cnc3c(c2)CN(c2nnc(C#N)c(C)c2C)CC3)c(F)n1] | 390.2 |
| 80 | 6-[3-[(3-fluoro-2-pyridyl)amino]- 7,8-dihydro-5H-1,6-naphthyridin-6- yl]-4,5-dimethyl-pyridazine-3- carbonitrile | 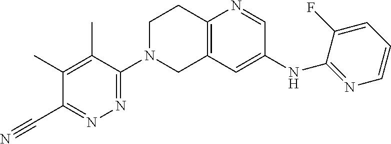 [SMILES: Cc1c(C#N)nnc(N2CCc3ncc(Nc4ncccc4F)cc3C2)c1C] | 376.2 |
| 81 | 6-[3-[(2-chloro-5-fluoro-pyrimidin- 4-yl)amino]-7,8-dihydro-5H-1,6- naphthyridin-6-yl]-4,5-dimethyl- pyridazine-3-carbonitrile | 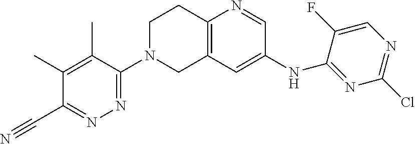 [SMILES: Cc1c(C#N)nnc(N2CCc3ncc(Nc4nc(Cl)ncc4F)cc3C2)c1C] | 411 |
| 82 | 6-[3-[(5-chloro-2-fluoro-3- pyridyl)amino]-7,8-dihydro-5H- 1,6-naphthyridin-6-yl]-4,5- dimethyl-pyridazine-3-carbonitrile | 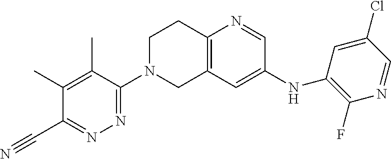 [SMILES: Cc1c(C#N)nnc(N2CCc3ncc(Nc4cc(Cl)cnc4F)cc3C2)c1C] | 410.2 |
| 83 | 6-(6-chloro-2,5-dimethyl- pyrimidin-4-yl)-7,8-dihydro-5H- 1,6-naphthyridine | 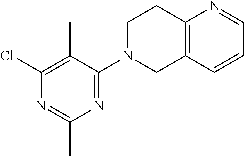 [SMILES: Cc1nc(Cl)c(C)c(N2CCc3ncccc3C2)n1] | 275.2 |
| 84 | 6-(6-chloro-2-cyclopropyl-5- methyl-pyrimidin-4-yl)-7,8- dihydro-5H-1,6-naphthyridine | 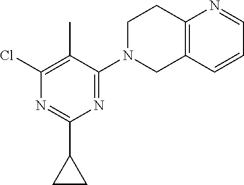 [SMILES: Cc1c(Cl)nc(C2CC2)nc1N1CCc2ncccc2C1] | 301.2 |
| 85 | 6-(6-chloro-2,5-dimethyl- pyrimidin-4-yl)-N-(2- fluorophenyl)-7,8-dihydro-5H-1,6- naphthyridin-3-amine | 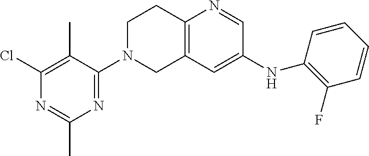 [SMILES: Cc1nc(Cl)c(C)c(N2CCc3ncc(Nc4ccccc4F)cc3C2)n1] | 384.2 |
| 86 | 6-(6-chloro-2,5-dimethyl- pyrimidin-4-yl)-N-(2,3- difluorophenyl)-7,8-dihydro-5H- 1,6-naphthyridin-3-amine | 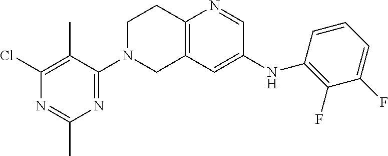 [SMILES: Cc1nc(Cl)c(C)c(N2CCc3ncc(Nc4cccc(F)c4F)cc3C2)n1] | 402.2 |
| 87 | 6-(6-chloro-2,5-dimethyl- pyrimidin-4-yl)-N-(2,4- difluorophenyl)-7,8-dihydro-5H- 1,6-naphthyridin-3-amine | 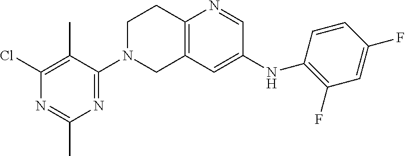 [SMILES: Cc1nc(Cl)c(C)c(N2CCc3ncc(Nc4ccc(F)cc4F)cc3C2)n1] | 402.2 |
| 88 | 6-(6-chloro-2,5-dimethyl- pyrimidin-4-yl)-N-(2,5- difluorophenyl)-7,8-dihydro-5H- 1,6-naphthyridin-3-amine | 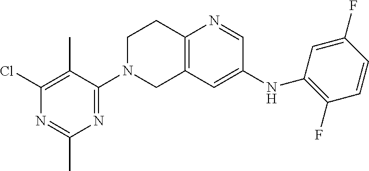 [SMILES: Cc1nc(Cl)c(C)c(N2CCc3ncc(Nc4cc(F)ccc4F)cc3C2)n1] | 402.2 |
| 89 | 6-(6-chloro-2,5-dimethyl- pyrimidin-4-yl)-N-(2,6- difluorophenyl)-7,8-dihydro-5H- 1,6-naphthyridin-3-amine | 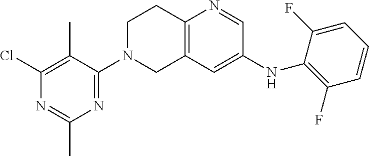 [SMILES: Cc1nc(Cl)c(C)c(N2CCc3ncc(Nc4c(F)cccc4F)cc3C2)n1] | 402.2 |
| 90 | 6-(6-chloro-2-cyclopropyl-5- methyl-pyrimidin-4-yl)-N-(2- fluorophenyl)-7,8-dihydro-5H-1,6- naphthyridin-3-amine | 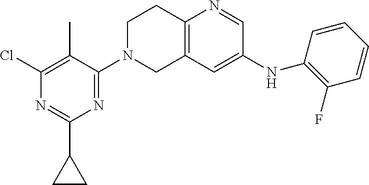 [SMILES: Cc1c(Cl)nc(C2CC2)nc1N1CCc2ncc(Nc3ccccc3F)cc2C1] | 410.2 |
| 91 | 6-(6-chloro-2-cyclopropyl-5- methyl-pyrimidin-4-yl)-N-(2,3- difluorophenyl)-7,8-dihydro-5H- 1,6-naphthyridin-3-amine | 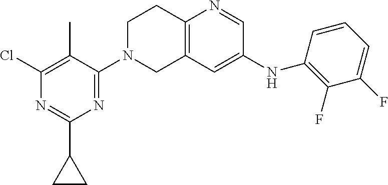 [SMILES: Cc1c(Cl)nc(C2CC2)nc1N1CCc2ncc(Nc3cccc(F)c3F)cc2C1] | 428.2 |
| 92 | 6-(6-chloro-2,5-dimethyl- pyrimidin-4-yl)-3-(1- methylpyrazol-4-yl)-7,8-dihydro- 5H-1,6-naphthyridine | 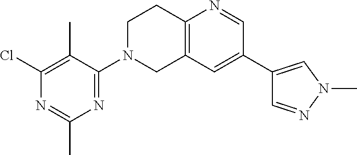 [SMILES: Cc1nc(Cl)c(C)c(N2CCc3ncc(-c4cnn(C)c4)cc3C2)n1] | 355.2 |
| 93 | 6-(6-chloro-2,5-dimethyl- pyrimidin-4-yl)-3-(2-methyl-4- pyridyl)-7,8-dihydro-5H-1,6- naphthyridine | 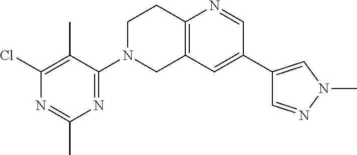 [SMILES: Cc1nc(Cl)c(C)c(N2CCc3ncc(-c4cnn(C)c4)cc3C2)n1] | 366.2 |
| 94 | 5-[6-(6-chloro-2,5-dimethyl- pyrimidin-4-yl)-7,8-dihydro-5H- 1,6-naphthyridin-3-yl]thiazole | 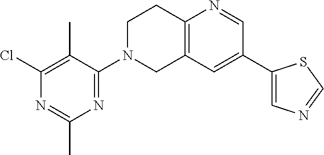 [SMILES: Cc1nc(Cl)c(C)c(N2CCc3ncc(-c4cncs4)cc3C2)n1] | 358.2 |
| 95 | 5-methyl-6-(3-thiazol-5-yl-7,8- dihydro-5H-1,6-naphthyridin-6- yl)pyridine-3-carbonitrile | 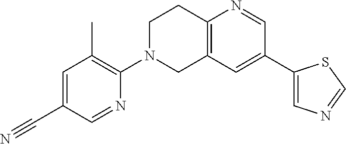 [SMILES: Cc1cc(C#N)cnc1N1CCc2ncc(-c3cncs3)cc2C1] | 334.4 |
| 96 | 6-[3-(1-cyclopropylpyrazol-4-yl)- 7,8-dihydro-5H-1,6-naphthyridin-6- yl]-5-methyl-pyridine-3- carbonitrile | 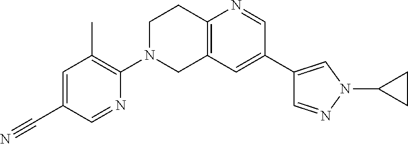 [SMILES: Cc1cc(C#N)cnc1N1CCc2ncc(-c3cnn(C4CC4)c3)cc2C1] | 357.2 |
| 97 | 6-[3-(1-benzylpyrazol-4-yl)-7,8- dihydro-5H-1,6-naphthyridin-6-yl]- 5-methyl-pyridine-3-carbonitrile | 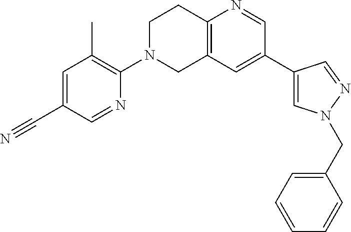 [SMILES: Cc1cc(C#N)cnc1N1CCc2ncc(-c3cnn(Cc4ccccc4)c3)cc2C1] | 407.4 |
| 98 | 6-[3-(1-cyclopentylpyrazol-4-yl)- 7,8-dihydro-5H-1,6-naphthyridin-6- yl]-5-methyl-pyridine-3- carbonitrile | 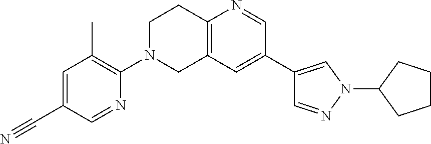 [SMILES: Cc1cc(C#N)cnc1N1CCc2ncc(-c3cnn(C4CCCC4)c3)cc2C1] | 385.4 |
| 99 | 6-[3-(1-cyclohexylpyrazol-4-yl)- 7,8-dihydro-5H-1,6-naphthyridin-6- yl]-5-methyl-pyridine-3- carbonitrile | 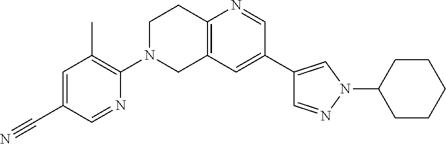 [SMILES: Cc1cc(C#N)cnc1N1CCc2ncc(-c3cnn(C4CCCCC4)c3)cc2C1] | 399.5 |
| 100 | 5-methyl-6-[3-[1-(2,2,2- trifluoroethyl)pyrazol-4-yl]-7,8- dihydro-5H-1,6-naphthyridin-6- yl]pyridine-3-carbonitrile | 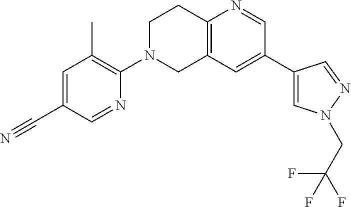 [SMILES: Cc1cc(C#N)cnc1N1CCc2ncc(-c3cnn(CC(F)(F)F)c3)cc2C1] | 399.3 |
| 101 | 6-[3-(1-cyclobutylpyrazol-4-yl)- 7,8-dihydro-5H-1,6-naphthyridin-6- yl]-5-methyl-pyridine-3- carbonitrile | 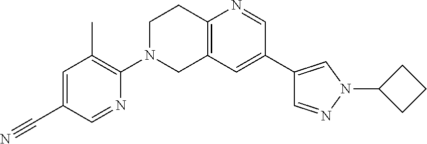 [SMILES: Cc1cc(C#N)cnc1N1CCc2ncc(-c3cnn(C4CCC4)c3)cc2C1] | 371.4 |
| 102 | 6-[3-[1- (cyclopropylmethyl)pyrazol-4-yl]- 7,8-dihydro-5H-1,6-naphthyridin-6- yl]-5-methyl-pyridine-3- carbonitrile | 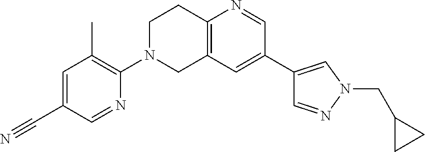 [SMILES: Cc1cc(C#N)cnc1N1CCc2ncc(-c3cnn(CC4CC4)c3)cc2C1] | 371.4 |
| 103 | 6-[3-(1,3-dimethylpyrazol-4-yl)- 7,8-dihydro-5H-1,6-naphthyridin-6- yl]-5-methyl-pyridine-3- carbonitrile | 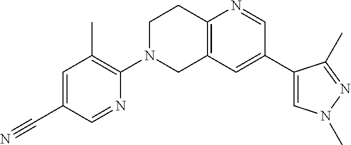 [SMILES: Cc1cc(C#N)cnc1N1CCc2ncc(-c3cn(C)nc3C)cc2C1] | 345.4 |
| 104 | 5-methyl-6-[3-(1-methylpyrazol-4- yl)-7,8-dihydro-5H-1,6- naphthyridin-6-yl]pyridine-3- carbonitrile | 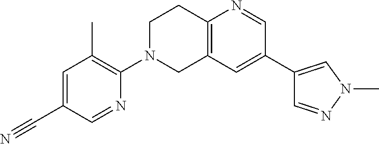 [SMILES: Cc1cc(C#N)cnc1N1CCc2ncc(-c3cnn(C)c3)cc2C1] | 331.4 |
| 105 | 6-[3-(1-ethylpyrazol-4-yl)-7,8- dihydro-5H-1,6-naphthyridin-6-yl]- 5-methyl-pyridine-3-carbonitrile | 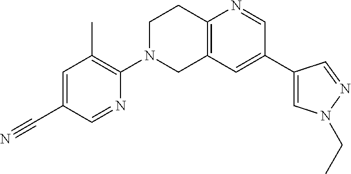 [SMILES: CCn1cc(-c2cnc3c(c2)CN(c2ncc(C#N)cc2C)CC3)cn1] | 345.3 |
| 106 | 6-[3-[1-(4-fluorophenyl)pyrazol-4- yl]-7,8-dihydro-5H-1,6- naphthyridin-6-yl]-5-methyl- pyridine-3-carbonitrile | 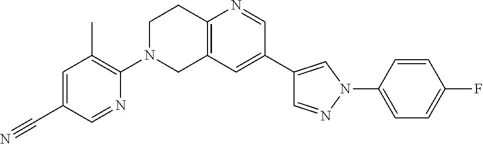 [SMILES: Cc1cc(C#N)cnc1N1CCc2ncc(-c3cnn(-c4ccc(F)cc4)c3)cc2C1] | 411.3 |
| 107 | 5-methyl-6-[3-(1,3,5- trimethylpyrazol-4-yl)-7,8-dihydro- 5H-1,6-naphthyridin-6-yl]pyridine- 3-carbonitrile | 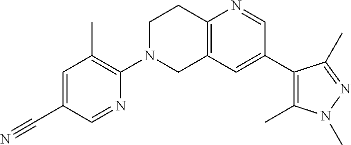 [SMILES: Cc1cc(C#N)cnc1N1CCc2ncc(-c3c(C)nn(C)c3C)cc2C1] | 359.2 |
| 108 | 5-methyl-6-[3-(1-propylpyrazol-4- yl)-7,8-dihydro-5H-1,6- naphthyridin-6-yl]pyridine-3- carbonitrile | 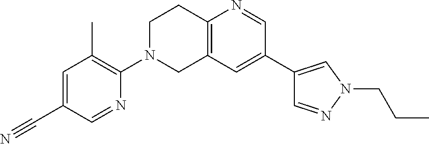 [SMILES: CCCn1cc(-c2cnc3c(c2)CN(c2ncc(C#N)cc2C)CC3)cn1] | 359.2 |
| 109 | 6-[3-(3,5-dimethylisoxazol-4-yl)- 7,8-dihydro-5H-1,6-naphthyridin-6- yl]-5-methyl-pyridine-3- carbonitrile | 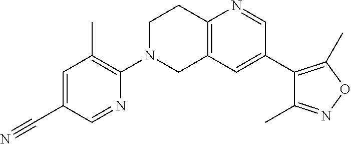 [SMILES: Cc1cc(C#N)cnc1N1CCc2ncc(-c3c(C)noc3C)cc2C1] | 346.2 |
| 110 | 6-[3-(2,5-difluoroanilino)-7,8- dihydro-5H-1,6-naphthyridin-6-yl]- 5-methyl-pyridine-3-carbonitrile | 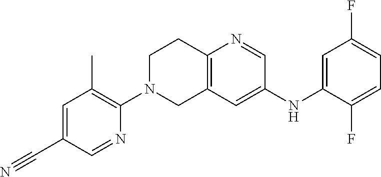 [SMILES: Cc1cc(C#N)cnc1N1CCc2ncc(Nc3cc(F)ccc3F)cc2C1] | 378.2 |
| 111 | 6-[3-(2-chloro-6-fluoro-anilino)- 7,8-dihydro-5H-1,6-naphthyridin-6- yl]-5-methyl-pyridine-3- carbonitrile | 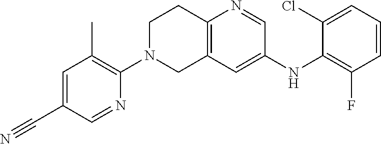 [SMILES: Cc1cc(C#N)cnc1N1CCc2ncc(Nc3c(F)cccc3Cl)cc2C1] | 394.2 |
| 112 | 6-[3-(1-isobutylpyrazol-4-yl)-7,8- dihydro-5H-1,6-naphthyridin-6-yl]- 5-methyl-pyridine-3-carbonitrile | 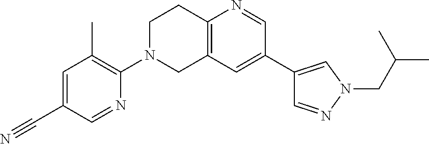 [SMILES: Cc1cc(C#N)cnc1N1CCc2ncc(-c3cnn(CC(C)C)c3)cc2C1] | 373.4 |
| 113 | 6-[3-[2-fluoro-3- (trifluoromethyl)anilino]-7,8- dihydro-5H-1,6-naphthyridin-6-yl]- 5-methyl-pyridine-3-carbonitrile | 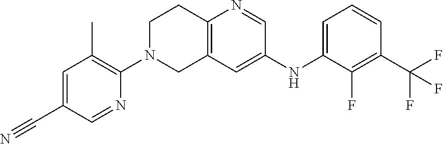 [SMILES: Cc1cc(C#N)cnc1N1CCc2ncc(Nc3cccc(C(F)(F)F)c3F)cc2C1] | 428.4 |
| 114 | 6-[3-(3-chloro-2-fluoro-anilino)- 7,8-dihydro-5H-1,6-naphthyridin-6- yl]-5-methyl-pyridine-3- carbonitrile | 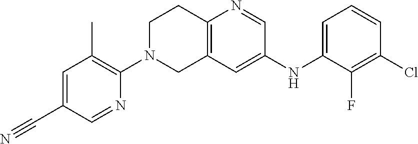 [SMILES: Cc1cc(C#N)cnc1N1CCc2ncc(Nc3cccc(Cl)c3F)cc2C1] | 394.3 |
| 115 | 6-[3-(2-fluoro-5-methyl-anilino)- 7,8-dihydro-5H-1,6-naphthyridin-6- yl]-5-methyl-pyridine-3- carbonitrile | 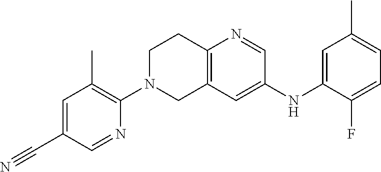 [SMILES: Cc1ccc(F)c(Nc2cnc3c(c2)CN(c2ncc(C#N)cc2C)CC3)c1] | 374.4 |
| 116 | 5-methyl-6-[3-(2,3,5- trifluoroanilino)-7,8-dihydro-5H- 1,6-naphthyridin-6-yl]pyridine-3- carbonitrile | 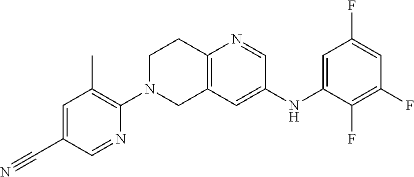 [SMILES: Cc1cc(C#N)cnc1N1CCc2ncc(Nc3cc(F)cc(F)c3F)cc2C1] | 396.4 |
| 117 | 4,5-dimethyl-6-(3-methyl-7,8- dihydro-5H-1,6-naphthyridin-6- yl)pyridazine-3-carbonitrile | 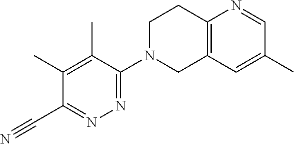 [SMILES: Cc1cnc2c(c1)CN(c1nnc(C#N)c(C)c1C)CC2] | 280.2 |
| 118 | 6-(7,8-dihydro-5H-1,6- naphthyridin-6-yl)-4,5-dimethyl- pyridazine-3-carbonitrile | 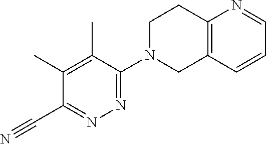 [SMILES: Cc1c(C#N)nnc(N2CCc3ncccc3C2)c1C] | 266.4 |
| 119 | 6-[3-[(2-fluoro-3-pyridyl)amino]- 7,8-dihydro-5H-1,6-naphthyridin-6- yl]-4,5-dimethyl-pyridazine-3- carbonitrile | 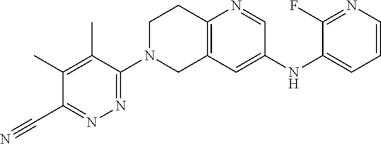 [SMILES: Cc1c(C#N)nnc(N2CCc3ncc(Nc4cccnc4F)cc3C2)c1C] | 376.2 |
| 120 | 6-(7,8-dihydro-5H-1,6- naphthyridin-6-yl)-N-(imidazo[1,2- alpyridin-2-ylmethyl)-5-methyl- pyridine-3-carboxamide | 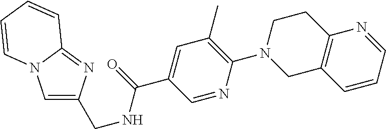 [SMILES: Cc1cc(C(=O)NCc2cn3ccccc3n2)cnc1N1CCc2ncccc2C1] | 399.2 |
| 121 | 6-(7,8-dihydro-5H-1,6- naphthyridin-6-yl)-5-methyl-N-(4- pyridyl)pyridine-3-carboxamide | 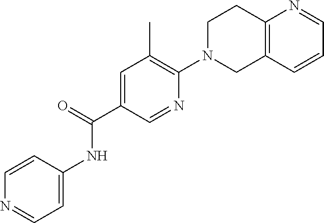 [SMILES: Cc1cc(C(=O)Nc2ccncc2)cnc1N1CCc2ncccc2C1] | 346.2 |
| 122 | 6-(7,8-dihydro-5H-1,6- naphthyridin-6-yl)-5-methyl-N- tetrahydropyran-4-yl-pyridine-3- carboxamide | 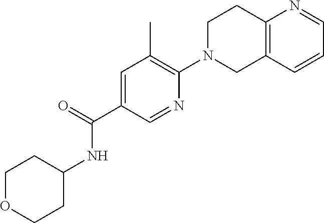 [SMILES: Cc1cc(C(=O)NC2CCOCC2)cnc1N1CCc2ncccc2C1] | 353.2 |
| 123 | 6-(7,8-dihydro-5H-1,6- naphthyridin-6-yl)-N-(isoxazol-5- ylmethyl)-5-methyl-pyridine-3- carboxamide | 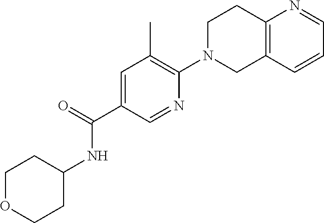 [SMILES: Cc1cc(C(=O)NC2CCOCC2)cnc1N1CCc2ncccc2C1] | 350.2 |
| 124 | 6-(7,8-dihydro-5H-1,6- naphthyridin-6-yl)-N-(3-fluoro-1- bicyclo[1.1.1]pentanyl)-5-methyl- pyridine-3-carboxamide | 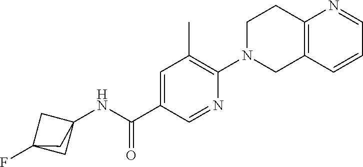 [SMILES: Cc1cc(C(=O)NC23CC(F)(C2)C3)cnc1N1CCc2ncccc2C1] | 353.2 |
| 125 | N-(1,3-benzothiazol-2-ylmethyl)-6- (7,8-dihydro-5H-1,6-naphthyridin- 6-yl)-5-methyl-pyridine-3- carboxamide | 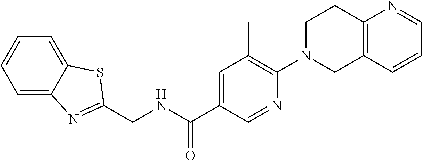 [SMILES: Cc1cc(C(=O)NCc2nc3ccccc3s2)cnc1N1CCc2ncccc2C1] | 416.2 |
| 126 | 6-(7,8-dihydro-5H-1,6- naphthyridin-6-yl)-5-methyl-N-[2- (4-pyridyl)ethyl]pyridine-3- carboxamide | 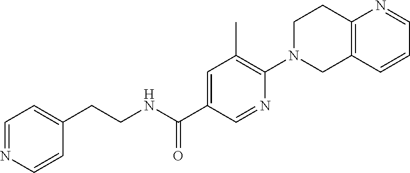 [SMILES: Cc1cc(C(=O)NCCc2ccncc2)cnc1N1CCc2ncccc2C1] | 374.2 |
| 127 | 6-(7,8-dihydro-5H-1,6- naphthyridin-6-yl)-5-methyl-N-[(2- methylthiazol-5- yl)methyl]pyridine-3-carboxamide | 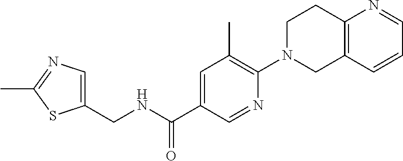 [SMILES: Cc1ncc(CNC(=O)c2cnc(N3CCc4ncccc4C3)c(C)c2)s1] | 380.2 |
| 128 | N-(benzothiophen-3-ylmethyl)-6- (7,8-dihydro-5H-1,6-naphthyridin- 6-yl)-5-methyl-pyridine-3- carboxamide | 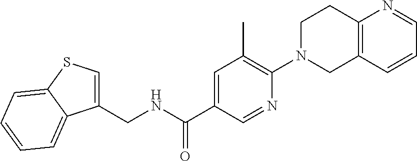 [SMILES: Cc1cc(C(=O)NCc2csc3ccccc23)cnc1N1CCc2ncccc2C1] | 415.2 |
| 129 | 6-(7,8-dihydro-5H-1,6- naphthyridin-6-yl)-5-methyl-N-[(2- methyl-4-pyridyl)methyl]pyridine- 3-carboxamide | 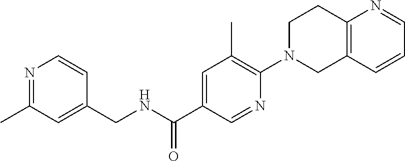 [SMILES: Cc1cc(CNC(=O)c2cnc(N3CCc4ncccc4C3)c(C)c2)ccn1] | 375.2 |
| 130 | 6-(7,8-dihydro-5H-1,6- naphthyridin-6-yl)-5-methyl-N-[(3- methyl-4-pyridyl)methyl]pyridine- 3-carboxamide | 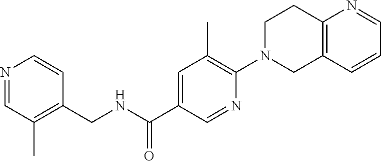 [SMILES: Cc1cnccc1CNC(=O)c1cnc(N2CCc3ncccc3C2)c(C)c1] | 374.2 |
| 131 | 6-(7,8-dihydro-5H-1,6- naphthyridin-6-yl)-5-methyl-N-(4- quinolylmethyl)pyridine-3- carboxamide | 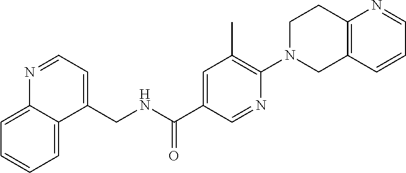 [SMILES: Cc1cc(C(=O)NCc2ccnc3ccccc23)cnc1N1CCc2ncccc2C1] | 410.2 |
| 132 | 6-[3-[(2-fluoro-6-methyl-3- pyridyl)amino]-7,8-dihydro-5H- 1,6-naphthyridin-6-yl]-5-methyl- pyridine-3-carbonitrile | 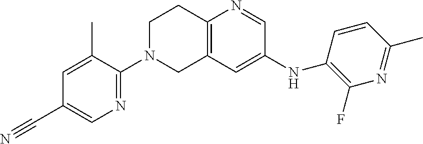 [SMILES: Cc1ccc(Nc2cnc3c(c2)CN(c2ncc(C#N)cc2C)CC3)c(F)n1] | 375.4 |
| 133 | methyl 6-(3-bromo-7,8-dihydro- 5H-1,6-naphthyridin-6-yl)-5- methyl-pyridine-3-carboxylate | 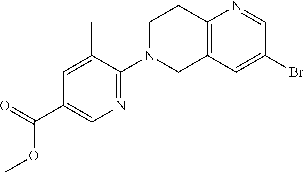 [SMILES: COC(=O)c1cnc(N2CCc3ncc(Br)cc3C2)c(C)c1] | 362 |
| 134 | N-[(2-chlorothiazol-5-yl)methyl]-6- (7,8-dihydro-5H-1,6-naphthyridin- 6-yl)-5-methyl-pyridine-3- carboxamide | 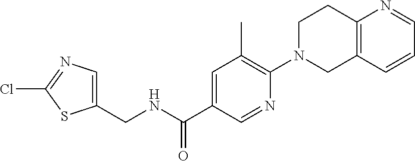 [SMILES: Cc1cc(C(=O)NCc2cnc(Cl)s2)cnc1N1CCc2ncccc2C1] | 400 |
| 135 | 6-(7,8-dihydro-5H-1,6- naphthyridin-6-yl)-4,5-dimethyl-N- (4-pyridylmethyl)pyridazine-3- carboxamide | 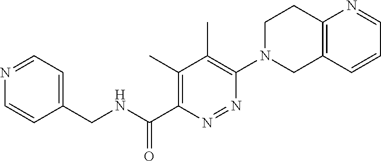 [SMILES: Cc1c(C(=O)NCc2ccncc2)nnc(N2CCc3ncccc3C2)c1C] | 375.3 |
| 136 | 6-(7,8-dihydro-5H-1,6- naphthyridin-6-yl)-4,5-dimethyl-N- (3-pyridylmethyl)pyridazine-3- carboxamide | 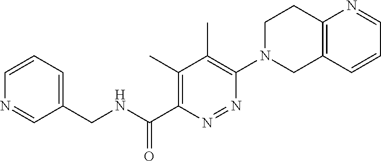 [SMILES: Cc1c(C(=O)NCc2cccnc2)nnc(N2CCc3ncccc3C2)c1C] | 375.3 |
| 137 | 6-(7,8-dihydro-5H-1,6- naphthyridin-6-yl)-4,5-dimethyl-N- (2-pyridylmethyl)pyridazine-3- carboxamide | 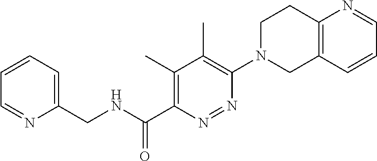 [SMILES: Cc1c(C(=O)NCc2ccccn2)nnc(N2CCc3ncccc3C2)c1C] | 375.4 |
| 138 | 6-(7,8-dihydro-5H-1,6- naphthyridin-6-yl)-4,5-dimethyl-N- (thiazol-5-ylmethyl)pyridazine-3- carboxamide | 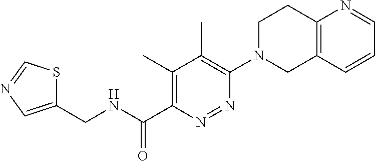 [SMILES: Cc1c(C(=O)NCc2cncs2)nnc(N2CCc3ncccc3C2)c1C] | 381.4 |
| 139 | N-[(5-bromo-3-pyridyl)methyl]-6- (7,8-dihydro-5H-1,6-naphthyridin- 6-yl)-5-methyl-pyridine-3- carboxamide | 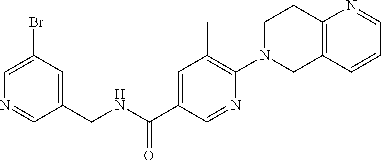 [SMILES: Cc1cc(C(=O)NCc2cncc(Br)c2)cnc1N1CCc2ncccc2C1] | 438.2 |
| 140 | 6-(7,8-dihydro-5H-1,6- naphthyridin-6-yl)-5-methyl-N-(3- quinolylmethyl)pyridine-3- carboxamide | 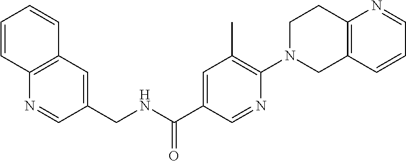 [SMILES: Cc1cc(C(=O)NCc2cnc3ccccc3c2)cnc1N1CCc2ncccc2C1] | 410.4 |
| 141 | N-[(5-bromo-2-methoxy- phenyl)methyl]-6-(7,8-dihydro-5H- 1,6-naphthyridin-6-yl)-5-methyl- pyridine-3-carboxamide | 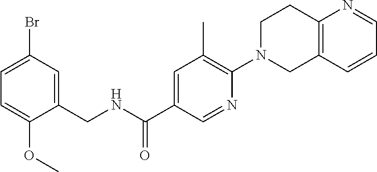 [SMILES: COc1ccc(Br)cc1CNC(=O)c1cnc(N2CCc3ncccc3C2)c(C)c1] | 467.3 |
| 142 | methyl 5-[[[6-(7,8-dihydro-5H-1,6- naphthyridin-6-yl)-5-methyl- pyridine-3- carbonyl]amino]methyl]pyridine-2- carboxylate | 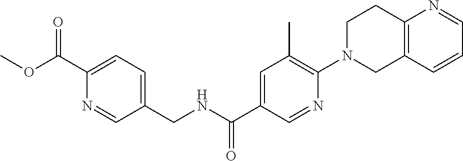 [SMILES: COC(=O)c1ccc(CNC(=O)c2cnc(N3CCc4ncccc4C3)c(C)c2)cn1] | 418.2 |
| 143 | 6-(7,8-dihydro-5H-1,6- naphthyridin-6-yl)-5-methyl-N-[(4- methylsulfonylphenyl)methyl] pyridine-3-carboxamide | 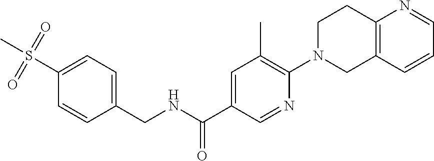 [SMILES: Cc1cc(C(=O)NCc2ccc(S(C)(=O)=O)cc2)cnc1N1CCc2ncccc2C1] | 437.2 |
| 144 | 6-(7,8-dihydro-5H-1,6- naphthyridin-6-yl)-5-methyl-N-[[5- (trifluoromethyl)-2- pyridyl]methyl]pyridine-3- carboxamide | 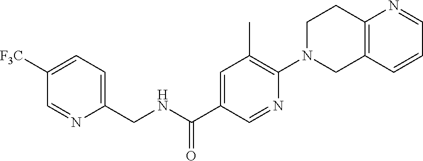 [SMILES: Cc1cc(C(=O)NCc2ccc(C(F)(F)F)cn2)cnc1N1CCc2ncccc2C1] | 428.2 |
| 145 | 6-(7,8-dihydro-5H-1,6- naphthyridin-6-yl)-5-methyl-N-[[5- (trifluoromethyl)pyrimidin-2- yl]methyl]pyridine-3-carboxamide | 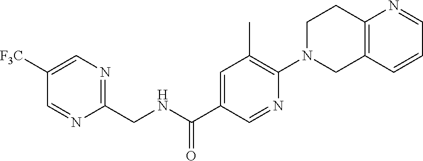 [SMILES: Cc1cc(C(=O)NCc2ncc(C(F)(F)F)cn2)cnc1N1CCc2ncccc2C1] | 429.2 |
| 146 | N-[(5-chloropyrimidin-2- yl)methyl]-6-(7,8-dihydro-5H-1,6- naphthyridin-6-yl)-5-methyl- pyridine-3-carboxamide | 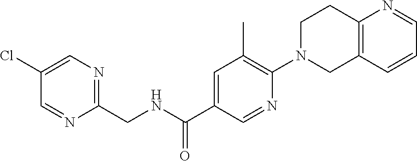 [SMILES: Cc1cc(C(=O)NCc2ncc(Cl)cn2)cnc1N1CCc2ncccc2C1] | 395.2 |
| 147 | 6-(7,8-dihydro-5H-1,6- naphthyridin-6-yl)-N-[(5- fluoropyrimidin-2-yl)methyl]-5- methyl-pyridine-3-carboxamide | 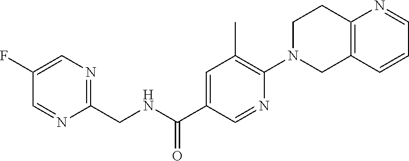 [SMILES: Cc1cc(C(=O)NCc2ncc(F)cn2)cnc1N1CCc2ncccc2C1] | 379.4 |
| 148 | 6-(7,8-dihydro-5H-1,6- naphthyridin-6-yl)-N-[(6-imidazol- 1-yl-3-pyridyl)methyl]-5-methyl- pyridine-3-carboxamide | 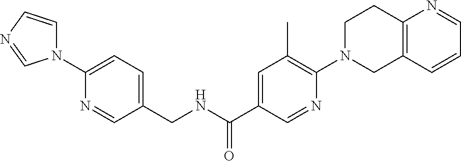 [SMILES: Cc1cc(C(=O)NCc2ccc(-n3ccnc3)nc2)cnc1N1CCc2ncccc2C1] | 426.3 |
| 149 | 6-(7,8-dihydro-5H-1,6- naphthyridin-6-yl)-5-methyl-N-[[4- (methylcarbamoyl)phenyl]methyl] pyridine-3-carboxamide | 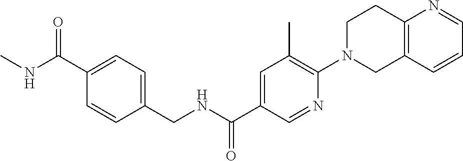 [SMILES: CNC(=O)c1ccc(CNC(=O)c2cnc(N3CCc4ncccc4C3)c(C)c2)cc1] | 416.3 |
| 150 | N-(3H-benzotriazol-5-ylmethyl)-6- (7,8-dihydro-5H-1,6-naphthyridin- 6-yl)-5-methyl-pyridine-3- carboxamide | 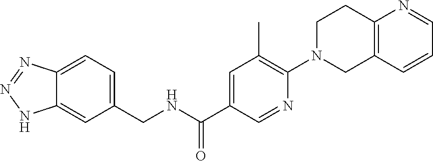 [SMILES: Cc1cc(C(=O)NCc2ccc3nn[nH]c3c2)cnc1N1CCc2ncccc2C1] | 400.4 |
| 151 | 6-(7,8-dihydro-5H-1,6- naphthyridin-6-yl)-5-methyl-N- ([1,2,4]triazolo[1,5-a]pyridin-6- ylmethyl)pyridine-3-carboxamide | 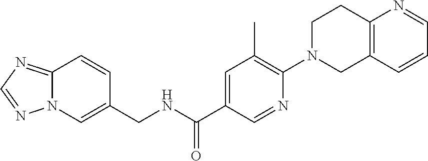 [SMILES: Cc1cc(C(=O)NCc2ccc3ncnn3c2)cnc1N1CCc2ncccc2C1] | 400.4 |
| 152 | 6-(7,8-dihydro-5H-1,6- naphthyridin-6-yl)-N-[(4-fluoro-3- methoxy-phenyl)methyl]-5-methyl- pyridine-3-carboxamide | 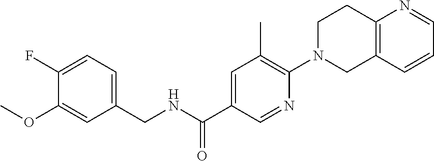 [SMILES: COc1cc(CNC(=O)c2cnc(N3CCc4ncccc4C3)c(C)c2)ccc1F] | 407.4 |
| 153 | N-(1,3-dihydroisobenzofuran-5- ylmethyl)-6-(7,8-dihydro-5H-1,6- naphthyridin-6-yl)-5-methyl- pyridine-3-carboxamide | 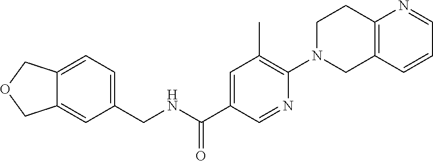 [SMILES: Cc1cc(C(=O)NCc2ccc3c(c2)COC3)cnc1N1CCc2ncccc2C1] | 401.4 |
| 154 | 6-(7,8-dihydro-5H-1,6- naphthyridin-6-yl)-5-methyl-N-[(2- oxoindolin-5-yl)methyl]pyridine-3- carboxamide | 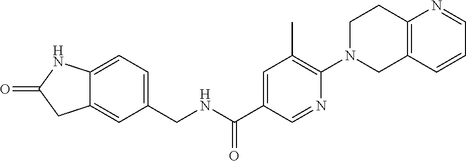 [SMILES: Cc1cc(C(=O)NCc2ccc3c(c2)CC(=O)N3)cnc1N1CCc2ncccc2C1] | 414.4 |
| 155 | N-(6,7-dihydro-5H- cyclopenta[b]pyridin-3-ylmethyl)- 6-(7,8-dihydro-5H-1,6- naphthyridin-6-yl)-5-methyl- pyridine-3-carboxamide | 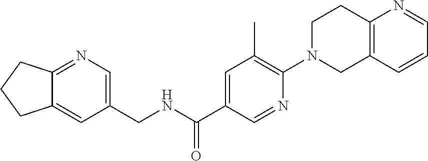 [SMILES: Cc1cc(C(=O)NCc2cnc3c(c2)CCC3)cnc1N1CCc2ncccc2C1] | 400.4 |
| 156 | 6-(7,8-dihydro-5H-1,6- naphthyridin-6-yl)-5-methyl-N-(8- quinolylmethyl)pyridine-3- carboxamide | 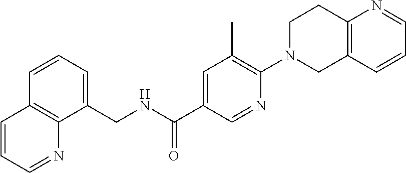 [SMILES: Cc1cc(C(=O)NCc2cccc3cccnc23)cnc1N1CCc2ncccc2C1] | 410.4 |
| 157 | 6-(7,8-dihydro-5H-1,6- naphthyridin-6-yl)-5-methyl-N- (quinoxalin-6-ylmethyl)pyridine-3- carboxamide | 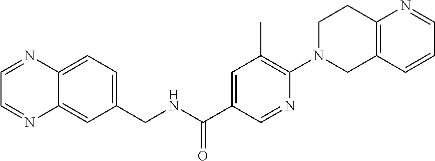 [SMILES: Cc1cc(C(=O)NCc2ccc3nccnc3c2)cnc1N1CCc2ncccc2C1] | 411.4 |
| 158 | 6-(7,8-dihydro-5H-1,6- naphthyridin-6-yl)-N-[(1,3- dimethyl-2-oxo-benzimidazol-5- yl)methyl]-5-methyl-pyridine-3- carboxamide | 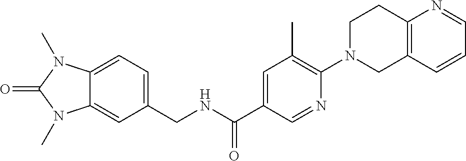 [SMILES: Cc1cc(C(=O)NCc2ccc3c(c2)n(C)c(=O)n3C)cnc1N1CCc2ncccc2C1] | 443.3 |
| 159 | 6-(7,8-dihydro-5H-1,6- naphthyridin-6-yl)-N-[(3- fluorophenyl)methyl]-5-methyl- pyridine-3-carboxamide | 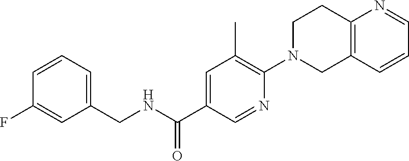 [SMILES: Cc1cc(C(=O)NCc2cccc(F)c2)cnc1N1CCc2ncccc2C1] | 377.4 |
| 160 | 6-(7,8-dihydro-5H-1,6- naphthyridin-6-yl)-5-methyl-N- (tetrahydropyran-4- ylmethyl)pyridine-3-carboxamide | 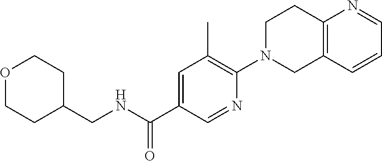 [SMILES: Cc1cc(C(=O)NCC2CCOCC2)cnc1N1CCc2ncccc2C1] | 367.4 |
| 161 | 6-(7,8-dihydro-5H-1,6- naphthyridin-6-yl)-5-methyl-N- (tetrahydropyran-2- ylmethyl)pyridine-3-carboxamide | 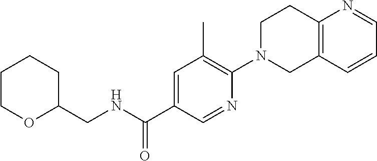 [SMILES: Cc1cc(C(=O)NCC2CCCCO2)cnc1N1CCc2ncccc2C1] | 367.5 |
| 162 | 6-(7,8-dihydro-5H-1,6- naphthyridin-6-yl)-N-[(7- methoxybenzofuran-2-yl)methyl]- 5-methyl-pyridine-3-carboxamide | 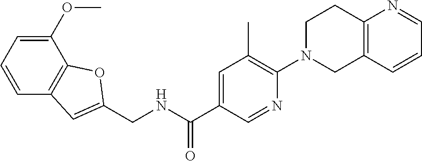 [SMILES: COc1cccc2cc(CNC(=O)c3cnc(N4CCc5ncccc5C4)c(C)c3)oc12] | 429.4 |
| 163 | 6-(7,8-dihydro-5H-1,6- naphthyridin-6-yl)-5-methyl-N- (1H-pyrrol-2-ylmethyl)pyridine-3- carboxamide | 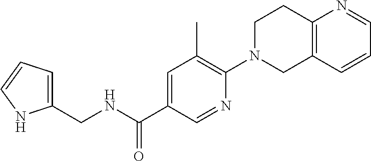 [SMILES: Cc1cc(C(=O)NCc2ccc[nH]2)cnc1N1CCc2ncccc2C1] | 348.4 |
| 164 | 6-(7,8-dihydro-5H-1,6- naphthyridin-6-yl)-N-[(1,1-dioxo- 2,3-dihydrobenzothiophen-5- yl)methyl]-5-methyl-pyridine-3- carboxamide | 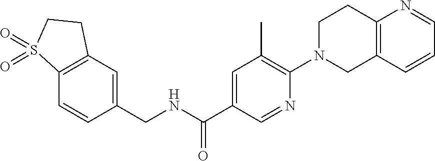 [SMILES: Cc1cc(C(=O)NCc2ccc3c(c2)CCS3(=O)=O)cnc1N1CCc2ncccc2C1] | 449.3 |
| 165 | 5-methyl-6-[3-(trifluoromethyl)- 7,8-dihydro-5H-1,6-naphthyridin-6- yl]pyridine-3-carbonitrile | 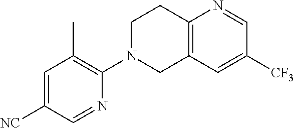 [SMILES: Cc1cc(C#N)cnc1N1CCc2ncc(C(F)(F)F)cc2C1] | 319.2 |
| 166 | 6-[3-[(2-fluoro-3-pyridyl)amino]- 7,8-dihydro-5H-1,6-naphthyridin-6- yl]-5-methyl-pyridine-3- carbonitrile | 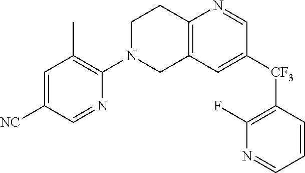 | 361.4 |
| 167 | 6-[3-[(3-fluoro-2-pyridyl)amino]- 7,8-dihydro-5H-1,6-naphthyridin-6- yl]-5-methyl-pyridine-3- carbonitrile | 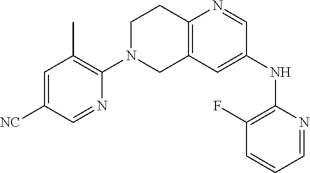 [SMILES: Cc1cc(C#N)cnc1N1CCc2ncc(Nc3ncccc3F)cc2C1] | 361.5 |
| 168 | 6-[3-(6-fluoro-2-pyridyl)-7,8- dihydro-5H-1,6-naphthyridin-6-yl]- 5-methyl-pyridine-3-carbonitrile | 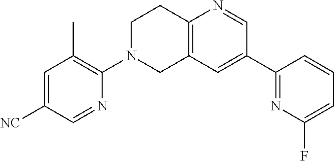 [SMILES: Cc1cc(C#N)cnc1N1CCc2ncc(-c3cccc(F)n3)cc2C1] | 346.4 |
| 169 | 4,5-dimethyl-6-[3-[(2- methylpyrazol-3-yl)amino]-7,8- dihydro-5H-1,6-naphthyridin-6- yl]pyridazine-3-carbonitrile | 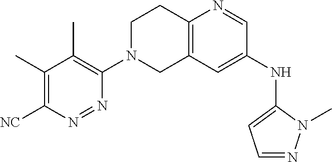 | 361.2 |
| 170 | 6-[3-(1,3-dimethylpyrazol-4-yl)- 7,8-dihydro-5H-1,6-naphthyridin-6- yl]-5-methyl-N-(thiazol-2- ylmethyl)pyridine-3-carboxamide | 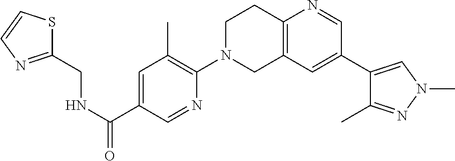 [SMILES: Cc1cc(C(=O)NCc2nccs2)cnc1N1CCc2ncc(-c3cn(C)nc3C)cc2C1] | 460.2 |
| 171 | 5-methyl-6-[3-(1-methylpyrazol-3- yl)-7,8-dihydro-5H-1,6- naphthyridin-6-yl]-N-(thiazol-2- ylmethyl)pyridine-3-carboxamide | 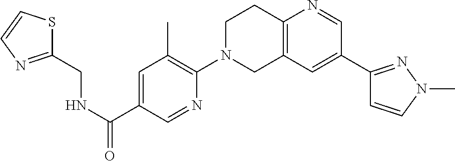 [SMILES: Cc1cc(C(=O)NCc2nccs2)cnc1N1CCc2ncc(-c3ccn(C)n3)cc2C1] | 446.2 |
| 172 | 6-[3-(3,5-dimethylisoxazol-4-yl)- 7,8-dihydro-5H-1,6-naphthyridin-6- yl]-5-methyl-N-(thiazol-2- ylmethyl)pyridine-3-carboxamide | 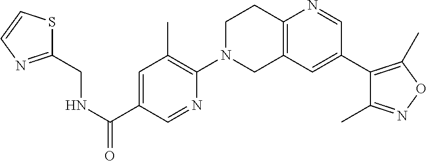 [SMILES: Cc1cc(C(=O)NCc2nccs2)cnc1N1CCc2ncc(-c3c(C)noc3C)cc2C1] | 461.2 |
| 173 | 5-methyl-6-[3-(1-methylpyrazol-4- yl)-7,8-dihydro-5H-1,6- naphthyridin-6-yl]-N-(thiazol-2- ylmethyl)pyridine-3-carboxamide | 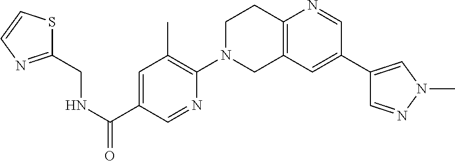 [SMILES: Cc1cc(C(=O)NCc2nccs2)cnc1N1CCc2ncc(-c3cnn(C)c3)cc2C1] | 446.2 |
| 174 | 6-[3-(6-fluoro-4-methyl-3-pyridyl)- 7,8-dihydro-5H-1,6-naphthyridin-6- yl]-5-methyl-N-(thiazol-2- ylmethyl)pyridine-3-carboxamide | 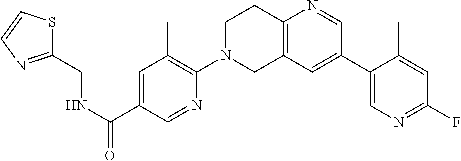 [SMILES: Cc1cc(F)ncc1-c1cnc2c(c1)CN(c1ncc(C(=O)NCc3nccs3)cc1C)CC2] | 475.2 |
| 175 | 6-[3-(2-fluoro-3-pyridyl)-7,8- dihydro-5H-1,6-naphthyridin-6-yl]- 5-methyl-N-(thiazol-2- ylmethyl)pyridine-3-carboxamide | 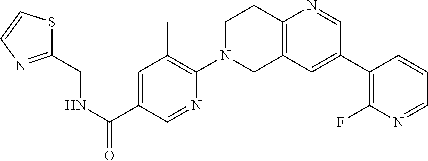 [SMILES: Cc1cc(C(=O)NCc2nccs2)cnc1N1CCc2ncc(-c3cccnc3F)cc2C1] | 461 |
| 176 | 5-methyl-6-[3-(1-methylpyrazol-4- yl)-7,8-dihydro-5H-1,6- naphthyridin-6-yl]-N-(4- pyridylmethyl)pyridine-3- carboxamide | 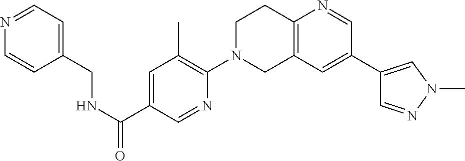 [SMILES: Cc1cc(C(=O)NCc2ccncc2)cnc1N1CCc2ncc(-c3cnn(C)c3)cc2C1] | 440.2 |
| 177 | 6-[3-(6-fluoro-4-methyl-3-pyridyl)- 7,8-dihydro-5H-1,6-naphthyridin-6- yl]-5-methyl-N-(4- pyridylmethyl)pyridine-3- carboxamide | 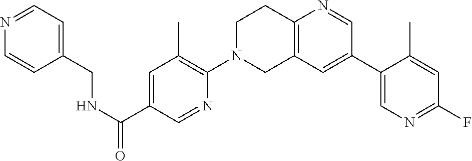 [SMILES: Cc1cc(F)ncc1-c1cnc2c(c1)CN(c1ncc(C(=O)NCc3ccncc3)cc1C)CC2] | 469.2 |
| 178 | 6-[3-(2-fluoro-3-pyridyl)-7,8- dihydro-5H-1,6-naphthyridin-6-yl]- 5-methyl-N-(4- pyridylmethyl)pyridine-3- carboxamide | 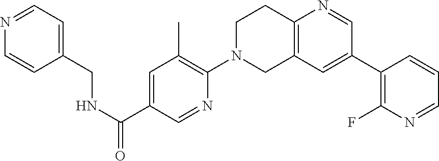 [SMILES: Cc1cc(C(=O)NCc2ccncc2)cnc1N1CCc2ncc(-c3cccnc3F)cc2C1] | 455.2 |
| 179 | 5-methyl-6-(3-methyl-7,8-dihydro- 5H-1,6-naphthyridin-6-yl)pyridine- 3-carbonitrile | 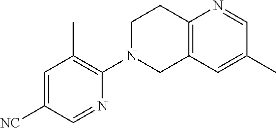 [SMILES: Cc1cnc2c(c1)CN(c1ncc(C#N)cc1C)CC2] | 265.4 |
| 180 | 6-[3-(2-fluorophenoxy)-7,8- dihydro-5H-1,6-naphthyridin-6-yl]- 5-methyl-pyridine-3-carbonitrile | 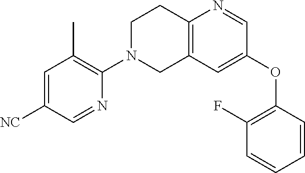 [SMILES: Cc1cc(C#N)cnc1N1CCc2ncc(Oc3ccccc3F)cc2C1] | 361.4 |
| 181 | 6-[3-(3-fluorophenoxy)-7,8- dihydro-5H-1,6-naphthyridin-6-yl]- 5-methyl-pyridine-3-carbonitrile | 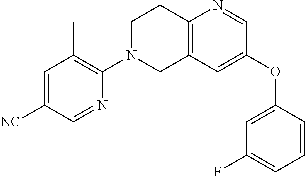 [SMILES: Cc1cc(C#N)cnc1N1CCc2ncc(Oc3cccc(F)c3)cc2C1] | 361.4 |
| 182 | 6-[3-[(6-methoxy-3-pyridyl)oxy]- 7,8-dihydro-5H-1,6-naphthyridin-6- yl]-5-methyl-pyridine-3- carbonitrile | 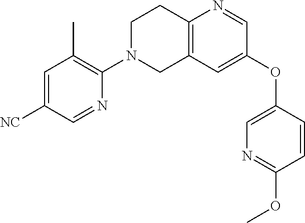 [SMILES: COc1ccc(Oc2cnc3c(c2)CN(c2ncc(C#N)cc2C)CC3)cn1] | 374.4 |
| 183 | 5-methyl-6-[3-(1-methylindazol-5- yl)oxy-7,8-dihydro-5H-1,6- naphthyridin-6-yl]pyridine-3- carbonitrile | 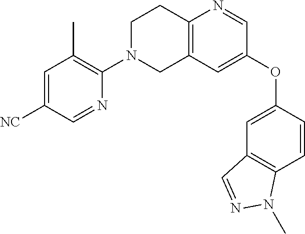 [SMILES: Cc1cc(C#N)cnc1N1CCc2ncc(Oc3ccc4c(cnn4C)c3)cc2C1] | 397.4 |
| 184 | 6-[3-(2,4-dimethylthiazol-5-yl)-7,8- dihydro-5H-1,6-naphthyridin-6-yl]- 5-methyl-pyridine-3-carbonitrile | 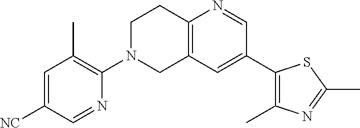 [SMILES: Cc1nc(C)c(-c2cnc3c(c2)CN(c2ncc(C#N)cc2C)CC3)s1] | 362.4 |
| 185 | 5-methyl-6-[3-(2-methylthiazol-5- yl)-7,8-dihydro-5H-1,6- naphthyridin-6-yl]pyridine-3- carbonitrile | 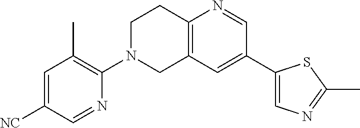 [SMILES: Cc1ncc(-c2cnc3c(c2)CN(c2ncc(C#N)cc2C)CC3)s1] | 348.4 |
| 186 | 6-[3-(6-cyclopropyl-3-pyridyl)-7,8- dihydro-5H-1,6-naphthyridin-6-yl]- 5-methyl-pyridine-3-carbonitrile | 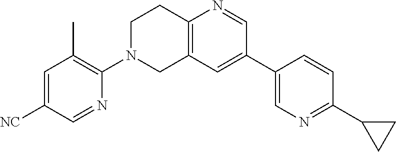 [SMILES: Cc1cc(C#N)cnc1N1CCc2ncc(-c3ccc(C4CC4)nc3)cc2C1] | 368.5 |
| 187 | 5-methyl-6-[3-(6-methyl-3- pyridyl)-7,8-dihydro-5H-1,6- naphthyridin-6-yl]pyridine-3- carbonitrile | 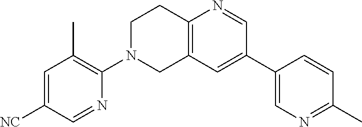 [SMILES: Cc1ccc(-c2cnc3c(c2)CN(c2ncc(C#N)cc2C)CC3)cn1] | 342.4 |
| 188 | 6-[3-(6-methoxy-3-pyridyl)-7,8- dihydro-5H-1,6-naphthyridin-6-yl]- 5-methyl-pyridine-3-carbonitrile | 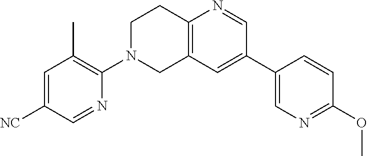 [SMILES: COc1ccc(-c2cnc3c(c2)CN(c2ncc(C#N)cc2C)CC3)cn1] | 358.4 |
| 189 | 4,5-dimethyl-6-[3-(2-methyl-4- pyridyl)-7,8-dihydro-5H-1,6- naphthyridin-6-yl]-N-(4- pyridylmethyl)pyridazine-3- carboxamide | 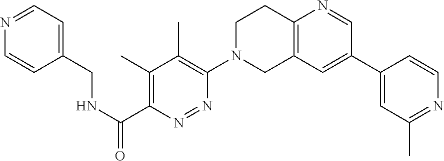 [SMILES: Cc1cc(-c2cnc3c(c2)CN(c2nnc(C(=O)NCc4ccncc4)c(C)c2C)CC3)ccn1] | 466.4 |
| 190 | 6-[3-(2-fluoroanilino)-7,8-dihydro- 5H-1,6-naphthyridin-6-yl]-4,5- dimethyl-N-(4- pyridylmethyl)pyridazine-3- carboxamide | 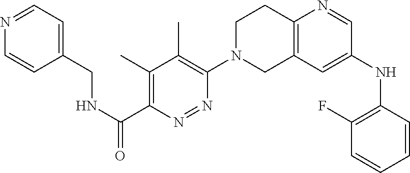 [SMILES: Cc1c(C(=O)NCc2ccncc2)nnc(N2CCc3ncc(Nc4ccccc4F)cc3C2)c1C] | 484.4 |
| 191 | 5-methyl-6-[3-(3-pyridyl)-7,8- dihydro-5H-1,6-naphthyridin-6- yl]pyridine-3-carbonitrile | 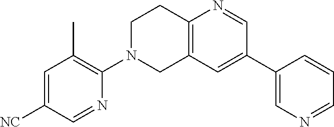 [SMILES: Cc1cc(C#N)cnc1N1CCc2ncc(-c3cccnc3)cc2C1] | 328.4 |
| 192 | 6-(6-chloro-2,5-dimethyl- pyrimidin-4-yl)-3-(1,3- dimethylpyrazol-4-yl)-7,8-dihydro- 5H-1,6-naphthyridine | 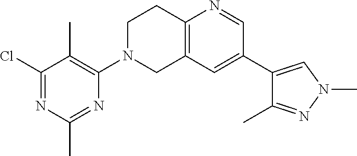 [SMILES: Cc1nc(Cl)c(C)c(N2CCc3ncc(-c4cn(C)nc4C)cc3C2)n1] | 369.2 |
| 193 | 6-(6-chloro-2,5-dimethyl- pyrimidin-4-yl)-3-(1,3,5- trimethylpyrazol-4-yl)-7,8-dihydro- 5H-1,6-naphthyridine | 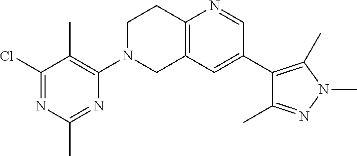 [SMILES: Cc1nc(Cl)c(C)c(N2CCc3ncc(-c4c(C)nn(C)c4C)cc3C2)n1] | 383.2 |
| 194 | 6-(6-chloro-2,5-dimethyl- pyrimidin-4-yl)-3-[1- (cyclopropylmethyl)pyrazol-4-yl]- 7,8-dihydro-5H-1,6-naphthyridine | 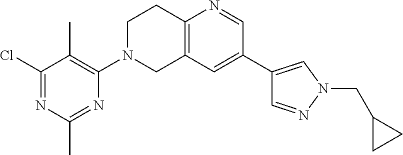 [SMILES: Cc1nc(Cl)c(C)c(N2CCc3ncc(-c4cnn(CC5CC5)c4)cc3C2)n1] | 395.2 |
| 195 | 6-(6-chloro-2,5-dimethyl- pyrimidin-4-yl)-3-(1- cyclobutylpyrazol-4-yl)-7,8- dihydro-5H-1,6-naphthyridine | 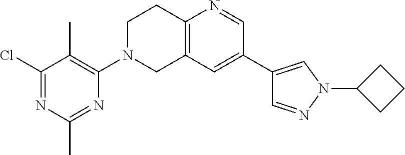 [SMILES: Cc1nc(Cl)c(C)c(N2CCc3ncc(-c4cnn(C5CCC5)c4)cc3C2)n1] | 395.2 |
| 196 | 3-(1,3-dimethylpyrazol-4-yl)-6-(6- methoxy-2,5-dimethyl-pyrimidin-4- yl)-7,8-dihydro-5H-1,6- naphthyridine | 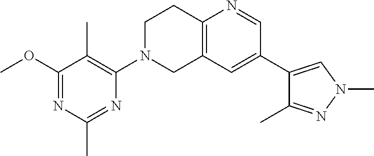 | 365.4 |
| 197 | 4-[6-(6-chloro-2,5-dimethyl- pyrimidin-4-yl)-7,8-dihydro-5H- 1,6-naphthyridin-3-yl]-3,5- dimethyl-isoxazole | 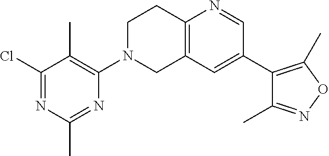 [SMILES: Cc1nc(Cl)c(C)c(N2CCc3ncc(-c4c(C)noc4C)cc3C2)n1] | 370.2 |
| 198 | 5-[6-(6-chloro-2,5-dimethyl- pyrimidin-4-yl)-7,8-dihydro-5H- 1,6-naphthyridin-3-yl]-2,4- dimethyl-thiazole | 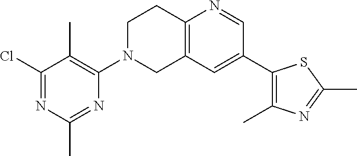 [SMILES: Cc1nc(Cl)c(C)c(N2CCc3ncc(-c4sc(C)nc4C)cc3C2)n1] | 386.2 |
| 199 | 5-[6-(6-chloro-2,5-dimethyl- pyrimidin-4-yl)-7,8-dihydro-5H- 1,6-naphthyridin-3-yl]-2-methyl- thiazole | 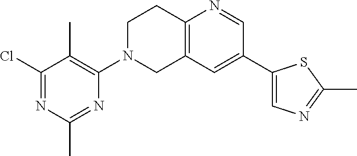 [SMILES: Cc1nc(Cl)c(C)c(N2CCc3ncc(-c4cnc(C)s4)cc3C2)n1] | 372.2 |
| 200 | 6-(6-chloro-2,5-dimethyl- pyrimidin-4-yl)-3- (trifluoromethyl)-7,8-dihydro-5H- 1,6-naphthyridine | 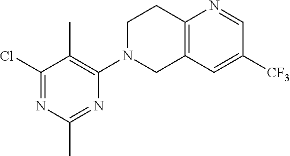 [SMILES: Cc1nc(Cl)c(C)c(N2CCc3ncc(C(F)(F)F)cc3C2)n1] | 343.2 |
| 201 | 3-(1,3-dimethylpyrazol-4-yl)-6- (2,5,6-trimethylpyrimidin-4-yl)-7,8- dihydro-5H-1,6-naphthyridine | 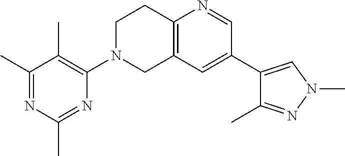 | 349.2 |
| 202 | 6-(6-chloro-2,5-dimethyl- pyrimidin-4-yl)-3-(4- fluorophenoxy)-7,8-dihydro-5H- 1,6-naphthyridine | 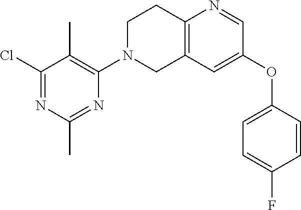 [SMILES: Cc1nc(Cl)c(C)c(N2CCc3ncc(Oc4ccc(F)cc4)cc3C2)n1] | 385.2 |
| 203 | 6-(6-chloro-2,5-dimethyl- pyrimidin-4-yl)-3-(3- fluorophenoxy)-7,8-dihydro-5H- 1,6-naphthyridine | 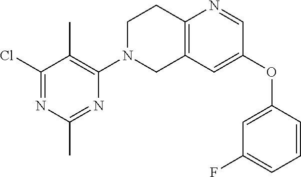 [SMILES: Cc1nc(Cl)c(C)c(N2CCc3ncc(Oc4cccc(F)c4)cc3C2)n1] | 385.2 |
| 204 | 6-(6-chloro-2,5-dimethyl- pyrimidin-4-yl)-3-(2- fluorophenoxy)-7,8-dihydro-5H- 1,6-naphthyridine | 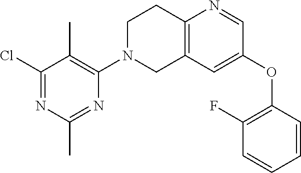 [SMILES: Cc1nc(Cl)c(C)c(N2CCc3ncc(Oc4ccccc4F)cc3C2)n1] | 385.2 |
| 205 | 6-(6-chloro-2,5-dimethyl- pyrimidin-4-yl)-3-[(6-methoxy-3- pyridyl)oxy]-7,8-dihydro-5H-1,6- naphthyridine | 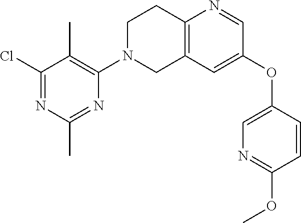 [SMILES: COc1ccc(Oc2cnc3c(c2)CN(c2nc(C)nc(Cl)c2C)CC3)cn1] | 398.2 |
| 206 | 6-(6-chloro-2,5-dimethyl- pyrimidin-4-yl)-3-(2,3-dihydro-1,4- benzodioxin-6-yloxy)-7,8-dihydro- 5H-1,6-naphthyridine | 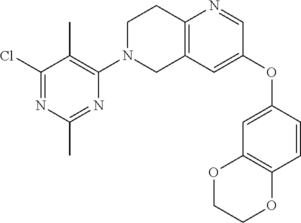 [SMILES: Cc1nc(Cl)c(C)c(N2CCc3ncc(Oc4ccc5c(c4)OCCO5)cc3C2)n1] | 425.2 |
| 207 | 6-(6-chloro-2,5-dimethyl- pyrimidin-4-yl)-3-(3-pyridyloxy)- 7,8-dihydro-5H-1,6-naphthyridine | 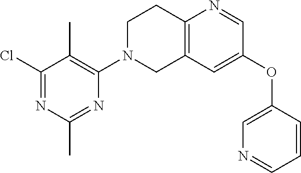 [SMILES: Cc1nc(Cl)c(C)c(N2CCc3ncc(Oc4cccnc4)cc3C2)n1] | 368.2 |
| 208 | 6-(6-chloro-2,5-dimethyl- pyrimidin-4-yl)-3-(1- methylindazol-5-yl)oxy-7,8- dihydro-5H-1,6-naphthyridine | 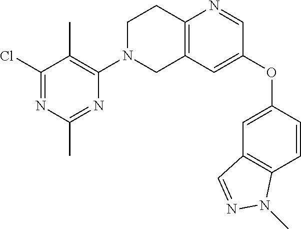 [SMILES: Cc1nc(Cl)c(C)c(N2CCc3ncc(Oc4ccc5c(cnn5C)c4)cc3C2)n1] | 421.2 |
| 209 | 6-(6-chloro-2,5-dimethyl- pyrimidin-4-yl)-3-methyl-7,8- dihydro-5H-1,6-naphthyridine | 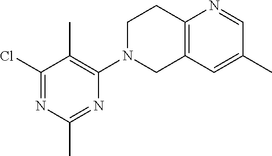 [SMILES: Cc1cnc2c(c1)CN(c1nc(C)nc(Cl)c1C)CC2] | 289.2 |
| 210 | 6-(6-chloro-2,5-dimethylpyrimidin- 4-yl)-3-(trifluoromethyl)-5,6,7,8- tetrahydro-1,6-naphthyridine | 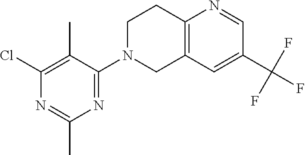 [SMILES: Cc1nc(Cl)c(C)c(N2CCc3ncc(C(F)(F)F)cc3C2)n1] | 343.2 |
| 211 | 3-bromo-6-(6-chloro-2- (methoxymethyl)-5- methylpyrimidin-4-yl)-5,6,7,8- tetrahydro-1,6-naphthyridine | 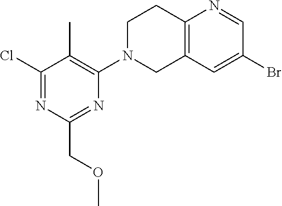 [SMILES: COCc1nc(Cl)c(C)c(N2CCc3ncc(Br)cc3C2)n1] | 385 |
| 212 | 6-(6-chloro-2,5-dimethylpyrimidin- 4-yl)-N-(1-methyl-1H-pyrazol-5- yl)-5,6,7,8-tetrahydro-1,6- naphthyridin-3-amine | 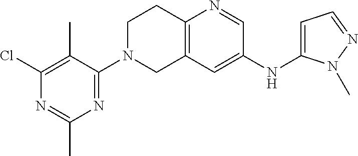 [SMILES: Cc1nc(Cl)c(C)c(N2CCc3ncc(Nc4ccnn4C)cc3C2)n1] | 370.2 |
| 213 | 6-(6-chloro-2,5-dimethylpyrimidin- 4-yl)-5,6,7,8-tetrahydro-1,6- naphthyridin-3-amine | 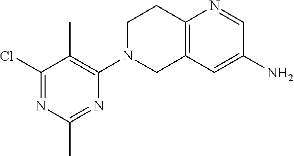 [SMILES: Cc1nc(Cl)c(C)c(N2CCc3ncc(N)cc3C2)n1] | 290.2 |
| 214 | 6-(6-chloro-2-(methoxymethyl)-5- methylpyrimidin-4-yl)-3- (trifluoromethyl)-5,6,7,8- tetrahydro-1,6-naphthyridine | 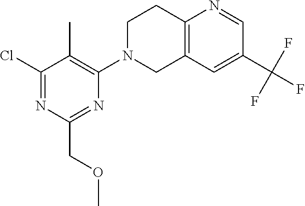 [SMILES: COCc1nc(Cl)c(C)c(N2CCc3ncc(C(F)(F)F)cc3C2)n1] | 373.2 |
| 215 | 6-(6-chloro-2,5-dimethylpyrimidin- 4-yl)-3-(difluoromethyl)-5,6,7,8- tetrahydro-1,6-naphthyridine | 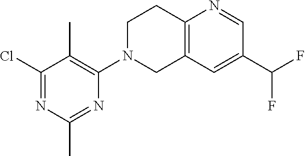 [SMILES: Cc1nc(Cl)c(C)c(N2CCc3ncc(C(F)F)cc3C2)n1] | 325.2 |
| 216 | 6-(6-chloro-2-(methoxymethyl)-5- methylpyrimidin-4-yl)-3-(1,3- dimethyl-1H-pyrazol-4-yl)-5,6,7,8- tetrahydro-1,6-naphthyridine | 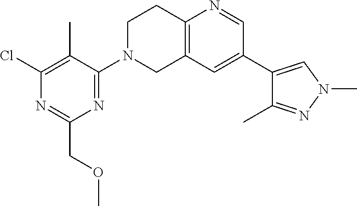 [SMILES: COCc1nc(Cl)c(C)c(N2CCc3ncc(-c4cn(C)nc4C)cc3C2)n1] | 399.2 |
| 217 | 5-(6-(6-chloro-2-(methoxymethyl)- 5-methylpyrimidin-4-yl)-5,6,7,8- tetrahydro-1,6-naphthyridin-3- yl)thiazole | 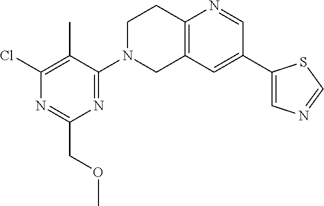 [SMILES: COCc1nc(Cl)c(C)c(N2CCc3ncc(-c4cncs4)cc3C2)n1] | 388.2 |
| 218 | 6-(6-chloro-2,5-dimethylpyrimidin- 4-yl)-2-(trifluoromethyl)-5,6,7,8- tetrahydro-1,6-naphthyridine | 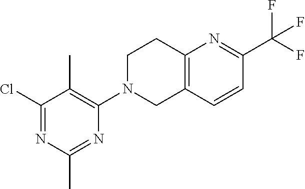 [SMILES: Cc1nc(Cl)c(C)c(N2CCc3nc(C(F)(F)F)ccc3C2)n1] | 343.3 |
| 219 | 6-(6-chloro-2,5-dimethylpyrimidin- 4-yl)-8,8-dimethyl-5,6,7,8- tetrahydro-1,6-naphthyridin-3- amine | 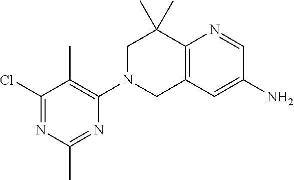 [SMILES: Cc1nc(Cl)c(C)c(N2Cc3cc(N)cnc3C(C)(C)C2)n1] | 318.4 |
| 220 | 5-(6-(6-chloro-2,5- dimethylpyrimidin-4-yl)-5,6,7,8- tetrahydro-1,6-naphthyridin-3-yl)- N,N-dimethylpyridin-2-amine | 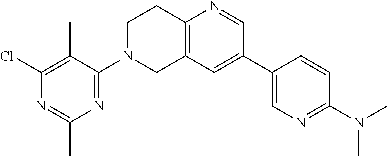 [SMILES: Cc1nc(Cl)c(C)c(N2CCc3ncc(-c4ccc(N(C)C)nc4)cc3C2)n1] | 395.4 |
| 221 | (2,5-dimethyl-6-(3- (trifluoromethyl)-7,8-dihydro-1,6- naphthyridin-6(5H)-yl)pyrimidin-4- yl)(morpholino)methanone | 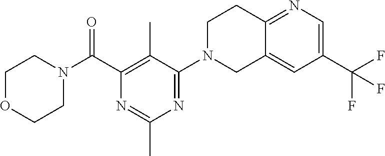 [SMILES: Cc1nc(C(=O)N2CCOCC2)c(C)c(N2CCc3ncc(C(F)(F)F)cc3C2)n1] | 422.3 |
| 222 | 6-(6-chloro-2,5-dimethylpyrimidin- 4-yl)-8-methyl-3-nitro-5,6,7,8- tetrahydro-1,6-naphthyridine | 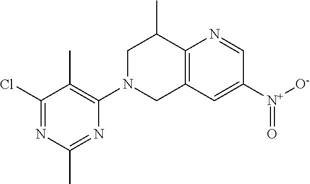 [SMILES: Cc1nc(Cl)c(C)c(N2Cc3cc([N+](=O)[O-])cnc3C(C)C2)n1] | 334.4 |
| 223 | 6-(2,5-dimethyl-6-(pyrrolidin-1- yl)pyrimidin-4-yl)-3-(6- fluoropyridin-3-yl)-5,6,7,8- tetrahydro-1,6-naphthyridine | 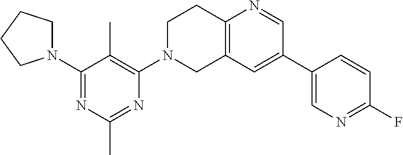 [SMILES: Cc1nc(N2CCCC2)c(C)c(N2CCc3ncc(-c4ccc(F)nc4)cc3C2)n1] | 405.4 |
| 224 | 6-(6-chloro-2,5-dimethylpyrimidin- 4-yl)-3-(6-fluoropyridin-3-yl)- 5,6,7,8-tetrahydro-1,6- naphthyridine | 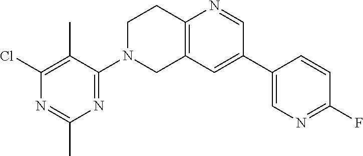 [SMILES: Cc1nc(Cl)c(C)c(N2CCc3ncc(-c4ccc(F)nc4)cc3C2)n1] | 370.3 |
| 225 | 6-(6-chloro-2,5-dimethylpyrimidin- 4-yl)-3-cyclopropyl-5,6,7,8- tetrahydro-1,6-naphthyridine | 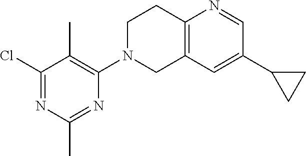 [SMILES: Cc1nc(Cl)c(C)c(N2CCc3ncc(C4CC4)cc3C2)n1] | 315.4 |
| 226 | (4-(3-(1,3-dimethyl-1H-pyrazol-4- yl)-7,8-dihydro-1,6-naphthyridin- 6(5H)-yl)-5,6-dimethylpyrimidin-2- yl)methanol | 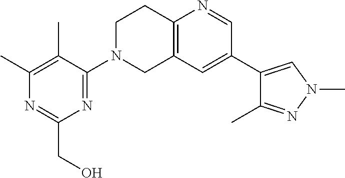 | 365.4 |
| 227 | 4-(5-(6-(2,5,6-trimethylpyrimidin- 4-yl)-5,6,7,8-tetrahydro-1,6- naphthyridin-3-yl)pyridin-2- yl)morpholine | 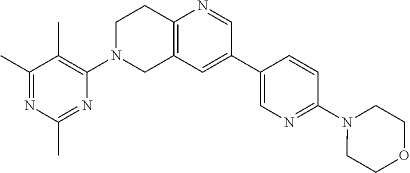 [SMILES: Cc1nc(C)c(C)c(N2CCc3ncc(-c4ccc(N5CCOCC5)nc4)cc3C2)n1] | 417.4 |
| 228 | 3-(6-(3,3-difluoropyrrolidin-1- yl)pyridin-3-yl)-6-(2,5,6- trimethylpyrimidin-4-yl)-5,6,7,8- tetrahydro-1,6-naphthyridine | 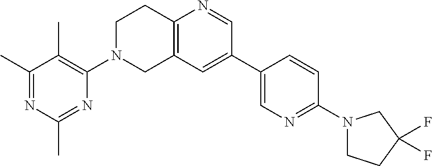 [SMILES: Cc1nc(C)c(C)c(N2CCc3ncc(-c4ccc(N5CCC(F)(F)C5)nc4)cc3C2)n1] | 437.4 |
| 229 | 3-(6-fluoropyridin-3-yl)-6-(2,5,6- trimethylpyrimidin-4-yl)-5,6,7,8- tetrahydro-1,6-naphthyridine | 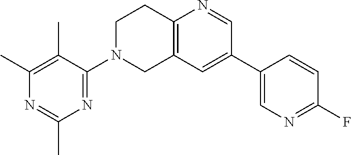 [SMILES: Cc1nc(C)c(C)c(N2CCc3ncc(-c4ccc(F)nc4)cc3C2)n1] | 350.5 |
| 230 | 3-(6-methoxypyridin-3-yl)-6- (2,5,6-trimethylpyrimidin-4-yl)- 5,6,7,8-tetrahydro-1,6- naphthyridine | 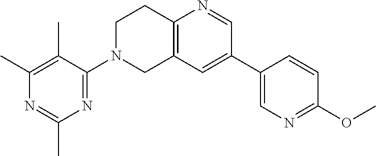 | 362.4 |
| 231 | 3-(6-(pyrrolidin-1-yl)pyridin-3-yl)- 6-(2,5,6-trimethylpyrimidin-4-yl)- 5,6,7,8-tetrahydro-1,6- naphthyridine | 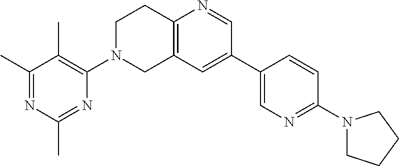 [SMILES: Cc1nc(C)c(C)c(N2CCc3ncc(-c4ccc(N5CCCC5)nc4)cc3C2)n1] | 401.4 |
| 232 | 3-(6-(1H-imidazol-1-yl)pyridin-3- yl)-6-(2,5,6-trimethylpyrimidin-4- yl)-5,6,7,8-tetrahydro-1,6- naphthyridine | 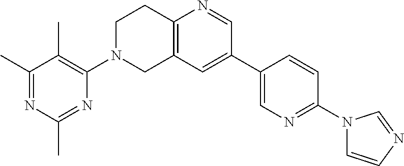 [SMILES: Cc1nc(C)c(C)c(N2CCc3ncc(-c4ccc(-n5ccnc5)nc4)cc3C2)n1] | 398.4 |
| 233 | 3-(6-(4-methylpiperazin-1- yl)pyridin-3-yl)-6-(2,5,6- trimethylpyrimidin-4-yl)-5,6,7,8- tetrahydro-1,6-naphthyridine | 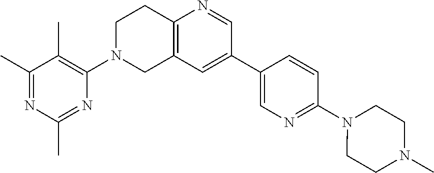 | 430.4 |
| 234 | 3-(6-(azetidin-1-yl)pyridin-3-yl)-6- (2,5,6-trimethylpyrimidin-4-yl)- 5,6,7,8-tetrahydro-1,6- naphthyridine | 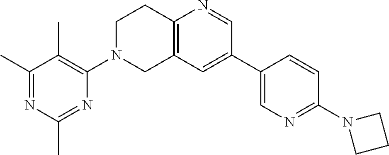 [SMILES: Cc1nc(C)c(C)c(N2CCc3ncc(-c4ccc(N5CCC5)nc4)cc3C2)n1] | 387.4 |
| 235 | 3-(trifluoromethyl)-6-(2,5,6- trimethylpyrimidin-4-yl)-5,6,7,8- tetrahydro-1,6-naphthyridine | 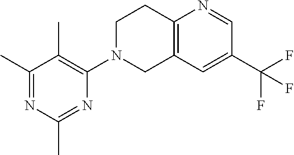 [SMILES: Cc1nc(C)c(C)c(N2CCc3ncc(C(F)(F)F)cc3C2)n1] | 323.3 |
| 236 | 3-bromo-6-(6-chloro-2,5- dimethylpyrimidin-4-yl)-8,8- dimethyl-5,6,7,8-tetrahydro-1,6- naphthyridine | 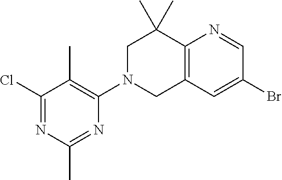 [SMILES: Cc1nc(Cl)c(C)c(N2Cc3cc(Br)cnc3C(C)(C)C2)n1] | 383.2 |
| 237 | 6-(6-chloro-5-methyl-2- (trifluoromethyl)pyrimidin-4-yl)-3- (trifluoromethyl)-5,6,7,8- tetrahydro-1,6-naphthyridine | 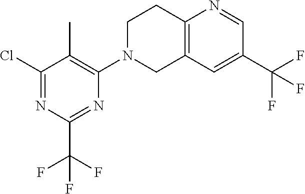 [SMILES: Cc1c(Cl)nc(C(F)(F)F)nc1N1CCc2ncc(C(F)(F)F)cc2C1] | 397.2 |
| 238 | 6-(6-chloro-2,5-dimethylpyrimidin- 4-yl)-3-(1,3-dimethyl-1H-pyrazol- 4-yl)-8,8-dimethyl-5,6,7,8- tetrahydro-1,6-naphthyridine | 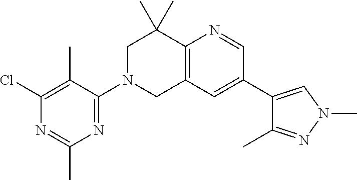 [SMILES: Cc1nc(Cl)c(C)c(N2Cc3cc(-c4cn(C)nc4C)cnc3C(C)(C)C2)n1] | 397.4 |
| 239 | 6-(6-chloro-2,5-dimethylpyrimidin- 4-yl)-8,8-dimethyl-N-(1-methyl- 1H-pyrazol-5-yl)-5,6,7,8- tetrahydro-1,6-naphthyridin-3- amine | 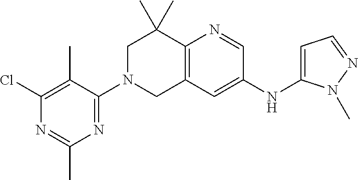 [SMILES: Cc1nc(Cl)c(C)c(N2Cc3cc(Nc4ccnn4C)cnc3C(C)(C)C2)n1] | 398.3 |
| 240 | N-(2-fluoropyridin-3-yl)-6-(2,5,6- trimethylpyrimidin-4-yl)-5,6,7,8- tetrahydro-1,6-naphthyridin-3- amine | 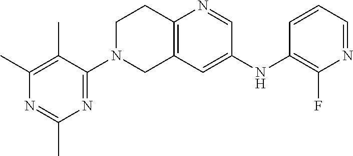 [SMILES: Cc1nc(C)c(C)c(N2CCc3ncc(Nc4cccnc4F)cc3C2)n1] | 365.4 |
| 241 | N-(1-methyl-1H-pyrazol-5-yl)-6- (2,5,6-trimethylpyrimidin-4-yl)- 5,6,7,8-tetrahydro-1,6- naphthyridin-3-amine | 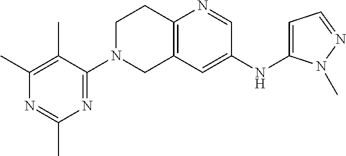 [SMILES: Cc1nc(C)c(C)c(N2CCc3ncc(Nc4ccnn4C)cc3C2)n1] | 350.4 |
| 242 | 6-(6-chloro-5-methyl-2- (trifluoromethyl)pyrimidin-4-yl)-3- cyclopropyl-5,6,7,8-tetrahydro-1,6- naphthyridine | 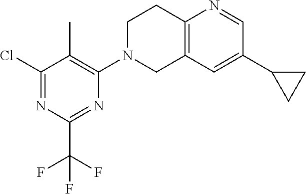 [SMILES: Cc1c(Cl)nc(C(F)(F)F)nc1N1CCc2ncc(C3CC3)cc2C1] | 369.3 |
| 243 | 6-(6-chloro-2,5-dimethylpyrimidin- 4-yl)-3-(6,7-dihydropyrazolo[1,5- a]pyrimidin-4(5H)-yl)-5,6,7,8- tetrahydro-1,6-naphthyridine | 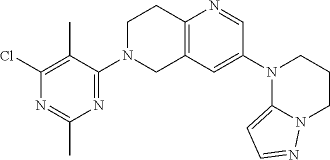 [SMILES: Cc1nc(Cl)c(C)c(N2CCc3ncc(N4CCCn5nccc54)cc3C2)n1] | 396.3 |
| 244 | 3-(6,7-dihydropyrazolo[1,5- a]pyrimidin-4(5H)-yl)-6-(2,5,6- trimethylpyrimidin-4-yl)-5,6,7,8- tetrahydro-1,6-naphthyridine | 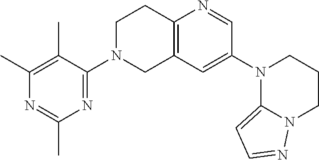 [SMILES: Cc1nc(C)c(C)c(N2CCc3ncc(N4CCCn5nccc54)cc3C2)n1] | 376.4 |
| 245 | 6-(6-chloro-5-methyl-2- (trifluoromethyl)pyrimidin-4-yl)-3- (1,3-dimethyl-1/-pyrazol-4-yl)- 5,6,7,8-tetrahydro-1,6- naphthyridine | 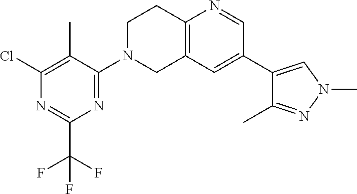 [SMILES: Cc1nn(C)cc1-c1cnc2c(c1)CN(c1nc(C(F)(F)F)nc(Cl)c1C)CC2] | 423.3 |
| 246 | 5-(6-(6-chloro-5-methyl-2- (trifluoromethyl)pyrimidin-4-yl)- 5,6,7,8-tetrahydro-1,6- naphthyridin-3-yl)-N,N- dimethylpyridin-2-amine | 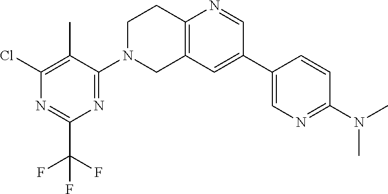 [SMILES: Cc1c(Cl)nc(C(F)(F)F)nc1N1CCc2ncc(-c3ccc(N(C)C)nc3)cc2C1] | 449.3 |
| 247 | 6-(6-chloro-5-methyl-2- (trifluoromethyl)pyrimidin-4-yl)-N- (1-methyl-1H-pyrazol-5-yl)- 5,6,7,8-tetrahydro-1,6- naphthyridin-3-amine | 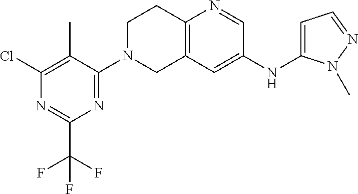 [SMILES: Cc1c(Cl)nc(C(F)(F)F)nc1N1CCc2ncc(Nc3ccnn3C)cc2C1] | 424.2 |
| 248 | 6-(6-chloro-5-methyl-2- (trifluoromethyl)pyrimidin-4-yl)-3- (6,7-dihydropyrazolo[1,5- a]pyrimidin-4(5H)-yl)-5,6,7,8- tetrahydro-1,6-naphthyridine | 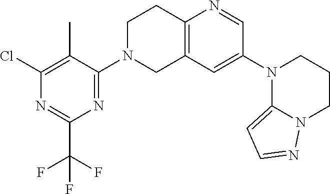 [SMILES: Cc1c(Cl)nc(C(F)(F)F)nc1N1CCc2ncc(N3CCCn4nccc43)cc2C1] | 450.2 |
| 249 | 6-(6-chloro-5-methyl-2- (trifluoromethyl)pyrimidin-4-yl)-N- (2-fluoropyridin-3-yl)-5,6,7,8- tetrahydro-1,6-naphthyridin-3- amine | 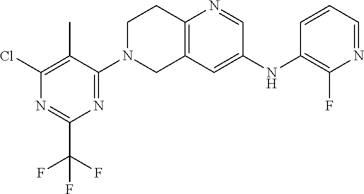 [SMILES: Cc1c(Cl)nc(C(F)(F)F)nc1N1CCc2ncc(Nc3cccnc3F)cc2C1] | 439.2 |
| 250 | 6-(6-chloro-2,5-dimethylpyrimidin- 4-yl)-3-(6,7-dihydropyrazolo[1,5- a]pyrimidin-4(5H)-yl)-8,8- dimethyl-5,6,7,8-tetrahydro-1,6- naphthyridine | 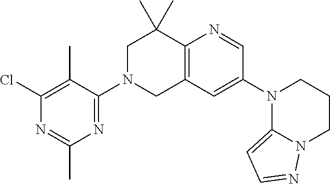 [SMILES: Cc1nc(Cl)c(C)c(N2Cc3cc(N4CCCn5nccc54)cnc3C(C)(C)C2)n1] | 424.3 |
| 251 | 6-(6-chloro-2,5-dimethylpyrimidin- 4-yl)-N-(2-fluoropyridin-3-yl)- 5,6,7,8-tetrahydro-1,6- naphthyridin-3-amine | 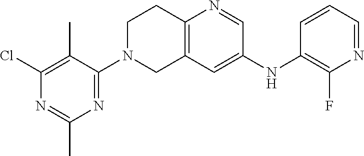 [SMILES: Cc1nc(Cl)c(C)c(N2CCc3ncc(Nc4cccnc4F)cc3C2)n1] | 385.3 |
| 252 | 6-(5,6-dimethyl-2- (trifluoromethyl)pyrimidin-4-yl)-3- (trifluoromethyl)-5,6,7,8- tetrahydro-1,6-naphthyridine | 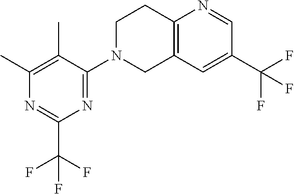 [SMILES: Cc1nc(C(F)(F)F)nc(N2CCc3ncc(C(F)(F)F)cc3C2)c1C] | 377.2 |
| 253 | 6-(2-chloro-5,6-dimethylpyrimidin- 4-yl)-3-(trifluoromethyl)-5,6,7,8- tetrahydro-1,6-naphthyridine | 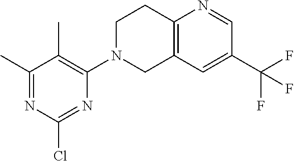 [SMILES: Cc1nc(Cl)nc(N2CCc3ncc(C(F)(F)F)cc3C2)c1C] | 343.2 |
| 254 | 6-(2-chloro-5,6-dimethylpyrimidin- 4-yl)-3-cyclopropyl-5,6,7,8- tetrahydro-1,6-naphthyridine | 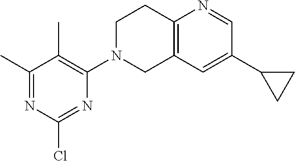 [SMILES: Cc1nc(Cl)nc(N2CCc3ncc(C4CC4)cc3C2)c1C] | 315.3 |
| 255 | 6-(6-chloro-2,5-dimethylpyrimidin- 4-yl)-8-methyl-5,6,7,8-tetrahydro- 1,6-naphthyridin-3-amine | 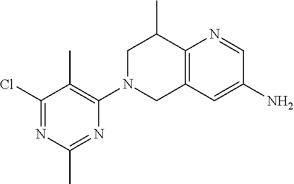 [SMILES: Cc1nc(Cl)c(C)c(N2Cc3cc(N)cnc3C(C)C2)n1] | 304.2 |
| 256 | 6-(6-chloro-2,5-dimethylpyrimidin- 4-yl)-8-methyl-3-(trifluoromethyl)- 5,6,7,8-tetrahydro-1,6- naphthyridine | 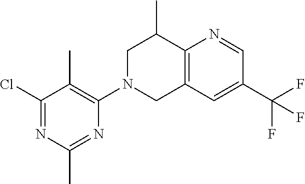 [SMILES: Cc1nc(Cl)c(C)c(N2Cc3cc(C(F)(F)F)cnc3C(C)C2)n1] | 357.2 |
| 257 | 6-(6-chloro-2,5-dimethylpyrimidin- 4-yl)-8,8-dimethyl-3- (trifluoromethyl)-5,6,7,8- tetrahydro-1,6-naphthyridine | 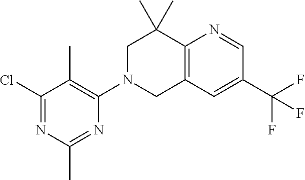 [SMILES: Cc1nc(Cl)c(C)c(N2Cc3cc(C(F)(F)F)cnc3C(C)(C)C2)n1] | 371.2 |
| 258 | 3-(6,7-dihydropyrazolo[1,5- a]pyrimidin-4(5H)-yl)-6-(5,6- dimethyl-2- (trifluoromethyl)pyrimidin-4-yl)- 5,6,7,8-tetrahydro-1,6- naphthyridine | 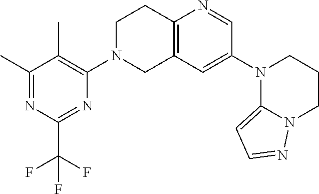 [SMILES: Cc1nc(C(F)(F)F)nc(N2CCc3ncc(N4CCCn5nccc54)cc3C2)c1C] | 430.4 |
| 259 | 6-(2-chloro-6,7-dihydro-5H- cyclopenta[d]pyrimidin-4-yl)-3- (trifluoromethyl)-5,6,7,8- tetrahydro-1,6-naphthyridine | 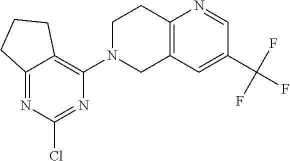 [SMILES: FC(F)(F)c1cnc2c(c1)CN(c1nc(Cl)nc3c1CCC3)CC2] | 355.2 |
| 260 | 3-(2-fluorophenoxy)-6-(2,5,6- trimethylpyrimidin-4-yl)-5,6,7,8- tetrahydro-1,6-naphthyridine | 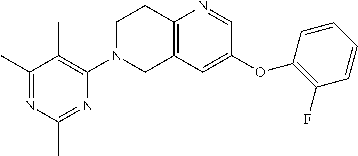 [SMILES: Cc1nc(C)c(C)c(N2CCc3ncc(Oc4ccccc4F)cc3C2)n1] | 365.4 |
| 261 | 3-(2,3-difluorophenoxy)-6-(2,5,6- trimethylpyrimidin-4-yl)-5,6,7,8- tetrahydro-1,6-naphthyridine | 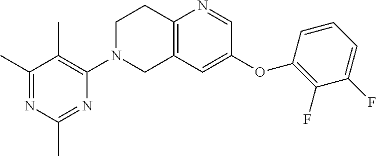 [SMILES: Cc1nc(C)c(C)c(N2CCc3ncc(Oc4cccc(F)c4F)cc3C2)n1] | 383.3 |
| 262 | 3-(2,4-difluorophenoxy)-6-(2,5,6- trimethylpyrimidin-4-yl)-5,6,7,8- tetrahydro-1,6-naphthyridine | 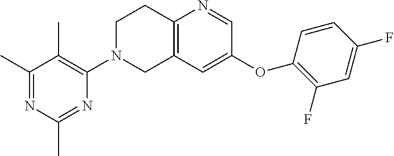 [SMILES: Cc1nc(C)c(C)c(N2CCc3ncc(Oc4ccc(F)cc4F)cc3C2)n1] | 383.3 |
| 263 | 3-(2,5-difluorophenoxy)-6-(2,5,6- trimethylpyrimidin-4-yl)-5,6,7,8- tetrahydro-1,6-naphthyridine | 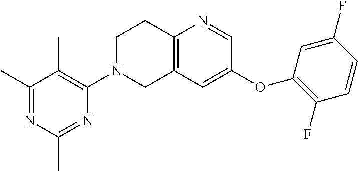 [SMILES: Cc1nc(C)c(C)c(N2CCc3ncc(Oc4cc(F)ccc4F)cc3C2)n1] | 383.3 |
| 264 | 2-chloro-4-(3-(trifluoromethyl)-7,8- dihydro-1,6-naphthyridin-6(5H)- yl)-5,6,7,8-tetrahydroquinazoline | 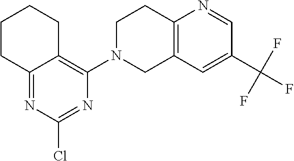 [SMILES: FC(F)(F)c1cnc2c(c1)CN(c1nc(Cl)nc3c1CCCC3)CC2] | 369.3 |
| 265 | 6-(2-cyclopropyl-6,7-dihydro-5H- cyclopenta[d]pyrimidin-4-yl)-3- (trifluoromethyl)-5,6,7,8- tetrahydro-1,6-naphthyridine | 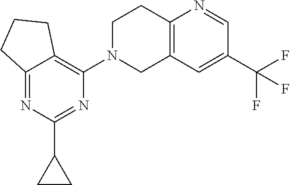 [SMILES: FC(F)(F)c1cnc2c(c1)CN(c1nc(C3CC3)nc3c1CCC3)CC2] | 361 |
| 266 | 2-cyclopropyl-4-(3- (trifluoromethyl)-7,8-dihydro-1,6- naphthyridin-6(5H)-yl)-5,6,7,8- tetrahydroquinazoline | 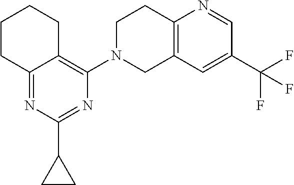 [SMILES: FC(F)(F)c1cnc2c(c1)CN(c1nc(C3CC3)nc3c1CCCC3)CC2] | 375 |
| 267 | 6-(2-methyl-6,7-dihydro-5H- cyclopenta[d]pyrimidin-4-yl)-3- (trifluoromethyl)-5,6,7,8- tetrahydro-1,6-naphthyridine | 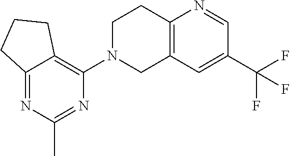 [SMILES: Cc1nc2c(c(N3CCc4ncc(C(F)(F)F)cc4C3)n1)CCC2] | 335 |
| 268 | 2-methyl-4-(3-(trifluoromethyl)- 7,8-dihydro-1,6-naphthyridin- 6(5H)-yl)-5,6,7,8- tetrahydroquinazoline | 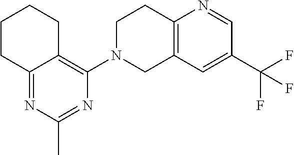 [SMILES: Cc1nc2c(c(N3CCc4ncc(C(F)(F)F)cc4C3)n1)CCCC2] | 349 |
| 269 | 3-(2-fluoro-4-methylphenoxy)-6- (2,5,6-trimethylpyrimidin-4-yl)- 5,6,7,8-tetrahydro-1,6- naphthyridine | 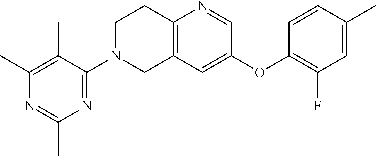 | 379 |
| 270 | 4-(4-(3-(1,3-dimethyl-1H-pyrazol- 4-yl)-7,8-dihydro-1,6-naphthyridin- 6(5H)-yl)-5,6-dimethylpyrimidin-2- yl)morpholine | 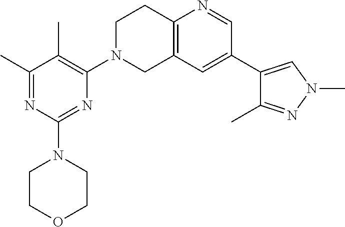 | 420.4 |
| 271 | 3-(1,3-dimethyl-1H-pyrazol-4-yl)- 6-(5,6-dimethyl-2-(pyrrolidin-1- yl)pyrimidin-4-yl)-5,6,7,8- tetrahydro-1,6-naphthyridine | 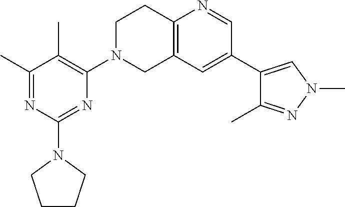 | 404.4 |
| 272 | N-cyclopropyl-4-(3-(1,3-dimethyl- 1H-pyrazol-4-yl)-7,8-dihydro-1,6- naphthyridin-6(5H)-yl)-5,6- dimethylpyrimidin-2-amine | 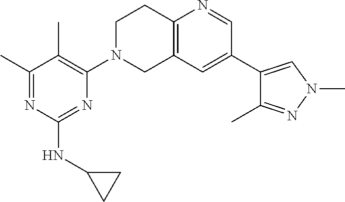 | 390.4 |
| 273 | 4-(3-(1,3-dimethyl-1H-pyrazol-4- yl)-7,8-dihydro-1,6-naphthyridin- 6(5H)-yl)-5,6-dimethyl-N- (tetrahydrofuran-3-yl)pyrimidin-2- amine | 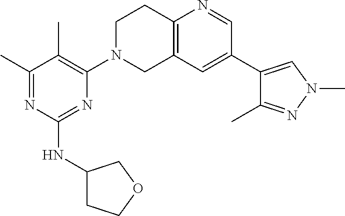 | 420.4 |
| 274 | 1-((4-(3-(1,3-dimethyl-1H-pyrazol- 4-yl)-7,8-dihydro-1,6-naphthyridin- 6(5H)-yl)-5,6-dimethylpyrimidin-2- yl)amino)-2-methylpropan-2-ol | 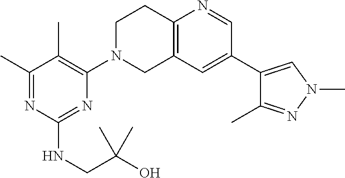 | 422.4 |
| 275 | 3-(1,3-dimethyl-1/-pyrazol-4-yl)- 6-(5,6-dimethyl-2-(4- methylpiperazin-1-yl)pyrimidin-4- yl)-5,6,7,8-tetrahydro-1,6- naphthyridine | 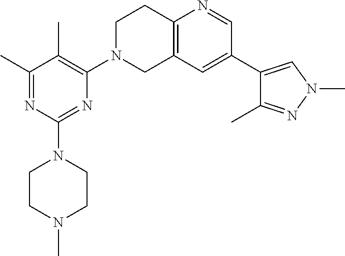 | 433.4 |
| 276 | 6-(5,6-dimethyl-2- morpholinopyrimidin-4-yl)-N-(1- methyl-1H-pyrazol-5-yl)-5,6,7,8- tetrahydro-1,6-naphthyridin-3- amine | 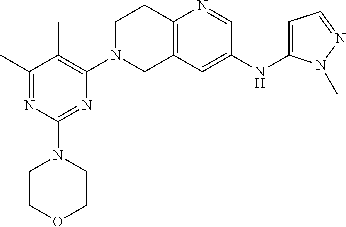 [SMILES: Cc1nc(N2CCOCC2)nc(N2CCc3ncc(Nc4ccnn4C)cc3C2)c1C] | 421.4 |
| 277 | 6-(5,6-dimethyl-2-(pyrrolidin-1- yl)pyrimidin-4-yl)-N-(1-methyl- 1H-pyrazol-5-yl)-5,6,7,8- tetrahydro-1,6-naphthyridin-3- amine | 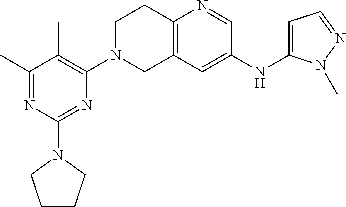 [SMILES: Cc1nc(N2CCCC2)nc(N2CCc3ncc(Nc4ccnn4C)cc3C2)c1C] | 405.4 |
| 278 | 6-(2-(cyclopropylamino)-5,6- dimethylpyrimidin-4-yl)-N-(1- methyl-1H-pyrazol-5-yl)-5,6,7,8- tetrahydro-1,6-naphthyridin-3- amine | 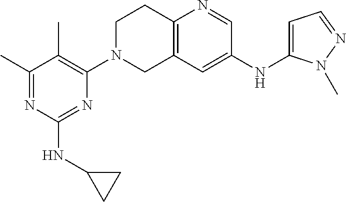 [SMILES: Cc1nc(NC2CC2)nc(N2CCc3ncc(Nc4ccnn4C)cc3C2)c1C] | 391.4 |
| 279 | 6-(5,6-dimethyl-2- ((tetrahydrofuran-3- yl)amino)pyrimidin-4-yl)-N-(1- methyl-1H-pyrazol-5-yl)-5,6,7,8- tetrahydro-1,6-naphthyridin-3- amine | 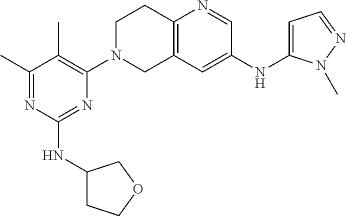 [SMILES: Cc1nc(NC2CCOC2)nc(N2CCc3ncc(Nc4ccnn4C)cc3C2)c1C] | 421.4 |
| 280 | 1-((4,5-dimethyl-6-(3-((1-methyl- 1H-pyrazol-5-yl)amino)-7,8- dihydro-1,6-naphthyridin-6(5/I)- yl)pyrimidin-2-yl)amino)-2- methylpropan-2-ol | 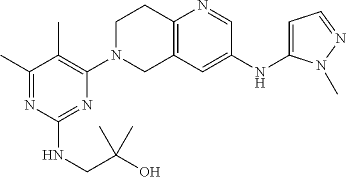 [SMILES: Cc1nc(NCC(C)(C)O)nc(N2CCc3ncc(Nc4ccnn4C)cc3C2)c1C] | 423.4 |
| 281 | 2-chloro-4-(3-(trifluoromethyl)-7,8- dihydro-1,6-naphthyridin-6(5H)- yl)-5,7-dihydrofuro[3,4- d]pyrimidine | 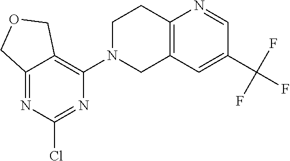 [SMILES: FC(F)(F)c1cnc2c(c1)CN(c1nc(Cl)nc3c1COC3)CC2] | 357.3 |
| 282 | 2-chloro-4-(3-(6,7- dihydropyrazolo[1,5-a]pyrimidin- 4(5H)-yl)-7,8-dihydro-1,6- naphthyridin-6(5H)-yl)-5,7- dihydrofuro[3,4-d]pyrimidine | 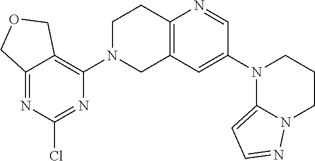 [SMILES: Clc1nc2c(c(N3CCc4ncc(N5CCCn6nccc65)cc4C3)n1)COC2] | 410.3 |
| 283 | 1-(6-(2-chloro-5,7-dihydrofuro[3,4- d]pyrimidin-4-yl)-5,6,7,8- tetrahydro-1,6-naphthyridin-3-yl)- 2,3-dihydro-1H-pyrido[2,3- b][1,4]oxazine | 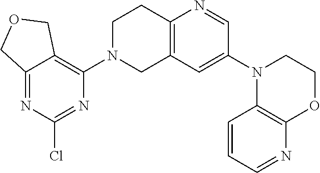 [SMILES: Clc1nc2c(c(N3CCc4ncc(N5CCOc6ncccc65)cc4C3)n1)COC2] | 423.2 |
| 284 | (R)-2-chloro-4-(3-(2- methylmorpholino)-7,8-dihydro- 1,6-naphthyridin-6(5H)-yl)-5,7- dihydrofuro[3,4-d]pyrimidine | 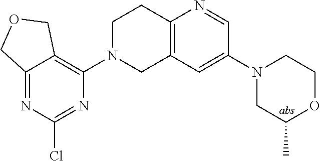 [SMILES: C[C@@H]1CN(c2cnc3c(c2)CN(c2nc(Cl)nc4c2COC4)CC3)CCO1] | 388.3 |
| 285 | 2-chloro-4-(3-(1,3-dimethyl-1H- pyrazol-4-yl)-7,8-dihydro-1,6- naphthyridin-6(5H)-yl)-5,7- dihydrofuro[3,4-d]pyrimidine | 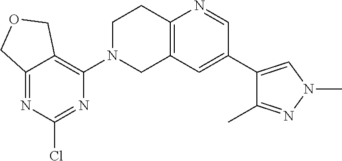 [SMILES: Cc1nn(C)cc1-c1cnc2c(c1)CN(c1nc(Cl)nc3c1COC3)CC2] | 383.2 |
| 286 | 2-chloro-4-(3-(1-cyclopropyl-1H- pyrazol-4-yl)-7,8-dihydro-1,6- naphthyridin-6(5H)-yl)-5,7- dihydrofuro[3,4-d]pyrimidine | 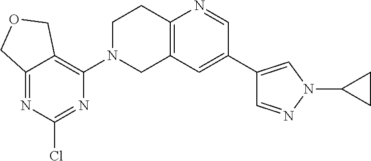 [SMILES: Clc1nc2c(c(N3CCc4ncc(-c5cnn(C6CC6)c5)cc4C3)n1)COC2] | 395.2 |
| 287 | 2-chloro-4-(3-cyclopropyl-7,8- dihydro-1,6-naphthyridin-6(5H)- yl)-5,7-dihydrofuro[3,4- d]pyrimidine | 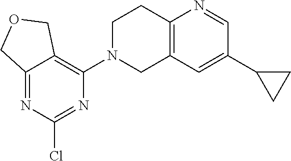 [SMILES: Clc1nc2c(c(N3CCc4ncc(C5CC5)cc4C3)n1)COC2] | 329.2 |
| 288 | 6-(2-chloro-5,7-dihydrofuro[3,4- d]pyrimidin-4-yl)-N-(1-methyl-1H- pyrazol-5-yl)-5,6,7,8-tetrahydro- 1,6-naphthyridin-3-amine | 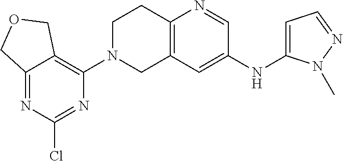 [SMILES: Cn1nccc1Nc1cnc2c(c1)CN(c1nc(Cl)nc3c1COC3)CC2] | 384.3 |
| 289 | 6-(2-chloro-5,7-dihydrofuro[3,4- d]pyrimidin-4-yl)-N-(2- fluoropyridin-3-yl)-5,6,7,8- tetrahydro-1,6-naphthyridin-3- amine | 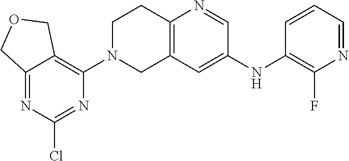 [SMILES: Fc1ncccc1Nc1cnc2c(c1)CN(c1nc(Cl)nc3c1COC3)CC2] | 399.2 |
| 290 | 2-chloro-4-(3-(2-fluorophenoxy)- 7,8-dihydro-1,6-naphthyridin- 6(5H)-yl)-5,7-dihydrofuro[3,4- d]pyrimidine | 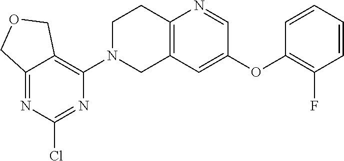 [SMILES: Fc1ccccc1Oc1cnc2c(c1)CN(c1nc(Cl)nc3c1COC3)CC2] | 399.3 |
| 291 | 6-(3-(isothiazol-5-yl)-7,8-dihydro- 1,6-naphthyridin-6(5H)-yl)-5- methylnicotinonitrile | 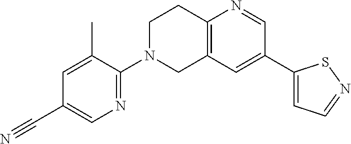 [SMILES: Cc1cc(C#N)cnc1N1CCc2ncc(-c3ccns3)cc2C1] | 334.4 |
| 292 | 6-(3-(isothiazol-5-yl)-7,8-dihydro- 1,6-naphthyridin-6(5H)-yl)-4,5- dimethylpyridazine-3-carbonitrile | 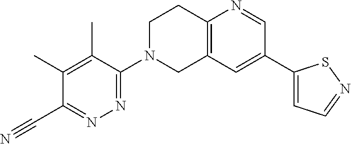 [SMILES: Cc1c(C#N)nnc(N2CCc3ncc(-c4ccns4)cc3C2)c1C] | 349.4 |
| 293 | 4,5-dimethyl-6-(3-(2-methylthiazol- 5-yl)-7,8-dihydro-1,6-naphthyridin- 6(5H)-yl)pyridazine-3-carbonitrile | 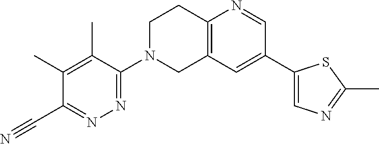 [SMILES: Cc1ncc(-c2cnc3c(c2)CN(c2nnc(C#N)c(C)c2C)CC3)s1] | 363.3 |
| 294 | 6-(3-(2,4-dimethylthiazol-5-yl)-7,8- dihydro-1,6-naphthyridin-6(5H)- yl)-4,5-dimethylpyridazine-3- carbonitrile | 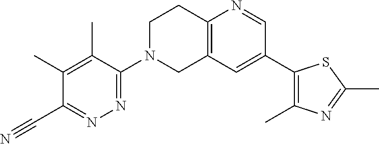 [SMILES: Cc1nc(C)c(-c2cnc3c(c2)CN(c2nnc(C#N)c(C)c2C)CC3)s1] | 377.4 |
| 295 | 6-(3-(1,3-dimethyl-1H-pyrazol-4- yl)-7,8-dihydro-1,6-naphthyridin- 6(5H)-yl)-4,5-dimethylpyridazine- 3-carbonitrile | 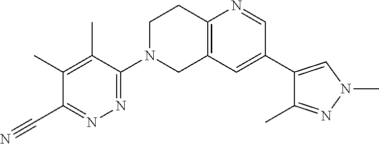 [SMILES: Cc1nn(C)cc1-c1cnc2c(c1)CN(c1nnc(C#N)c(C)c1C)CC2] | 360.4 |
| 296 | 4,5-dimethyl-6-(3-(1,3,5-trimethyl- 1H-pyrazol-4-yl)-7,8-dihydro-1,6- naphthyridin-6(5H)-yl)pyridazine- 3-carbonitrile | 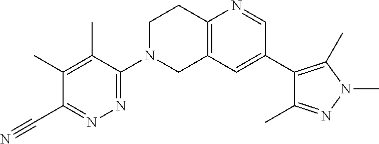 [SMILES: Cc1nn(C)c(C)c1-c1cnc2c(c1)CN(c1nnc(C#N)c(C)c1C)CC2] | 374.4 |
| 297 | 6-(3-(3,5-dimethylisoxazol-4-yl)- 7,8-dihydro-1,6-naphthyridin- 6(5H)-yl)-4,5-dimethylpyridazine- 3-carbonitrile | 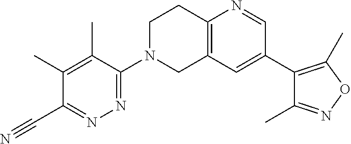 [SMILES: Cc1noc(C)c1-c1cnc2c(c1)CN(c1nnc(C#N)c(C)c1C)CC2] | 361.4 |
| 298 | 4,5-dimethyl-6-(3-(6- methylpyridin-3-yl)-7,8-dihydro- 1,6-naphthyridin-6(5H)- yl)pyridazine-3-carbonitrile | 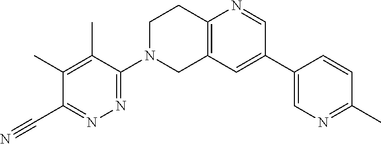 [SMILES: Cc1ccc(-c2cnc3c(c2)CN(c2nnc(C#N)c(C)c2C)CC3)cn1] | 357.4 |
| 299 | 6-(3-(6-methoxypyridin-3-yl)-7,8- dihydro-1,6-naphthyridin-6(5H)- yl)-4,5-dimethylpyridazine-3- carbonitrile | 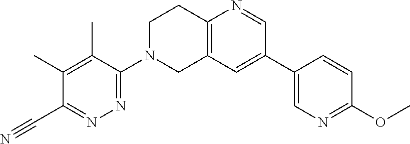 [SMILES: COc1ccc(-c2cnc3c(c2)CN(c2nnc(C#N)c(C)c2C)CC3)cn1] | 373.4 |
| 300 | 6-(3-(4-fluorophenoxy)-7,8- dihydro-1,6-naphthyridin-6(5H)- yl)-4,5-dimethylpyridazine-3- carbonitrile | 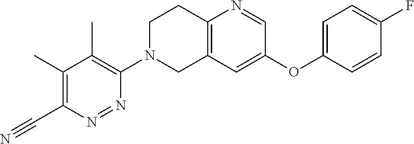 [SMILES: Cc1c(C#N)nnc(N2CCc3ncc(Oc4ccc(F)cc4)cc3C2)c1C] | 376.4 |
| 301 | 6-(3-(6-cyclopropylpyridin-3-yl)- 7,8-dihydro-1,6-naphthyridin- 6(5H)-yl)-4,5-dimethylpyridazine- 3-carbonitrile | 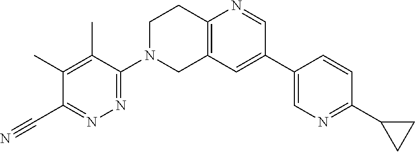 [SMILES: Cc1c(C#N)nnc(N2CCc3ncc(-c4ccc(C5CC5)nc4)cc3C2)c1C] | 383.4 |
| 302 | 6-(3-((6-methoxypyridin-3-yl)oxy)- 7,8-dihydro-1,6-naphthyridin- 6(5H)-yl)-4,5-dimethylpyridazine- 3-carbonitrile | 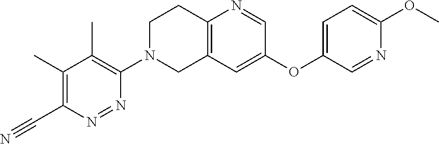 [SMILES: COc1ccc(Oc2cnc3c(c2)CN(c2nnc(C#N)c(C)c2C)CC3)cn1] | 389.4 |
| 303 | 4,5-dimethyl-6-(3- (trifluoromethyl)-7,8-dihydro-1,6- naphthyridin-6(5H)-yl)pyridazine- 3-carbonitrile | 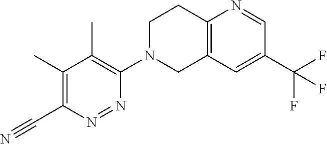 [SMILES: Cc1c(C#N)nnc(N2CCc3ncc(C(F)(F)F)cc3C2)c1C] | 334.2 |
| 304 | 5-methyl-6-(3-(trifluoromethyl)- 7,8-dihydro-1,6-naphthyridin- 6(5H)-yl)pyridazine-3-carbonitrile | 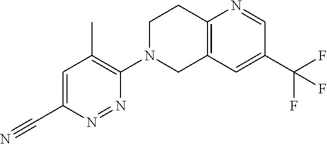 [SMILES: Cc1cc(C#N)nnc1N1CCc2ncc(C(F)(F)F)cc2C1] | 320.2 |
| 305 | 5-methyl-6-(3-methyl-7,8-dihydro- 1,6-naphthyridin-6(5H)- yl)pyridazine-3-carbonitrile | 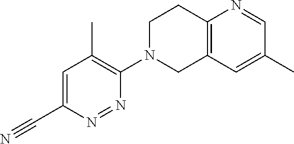 [SMILES: Cc1cnc2c(c1)CN(c1nnc(C#N)cc1C)CC2] | 266.2 |
| 306 | 5-methyl-6-(3-((1-methyl-1H- pyrazol-5-yl)amino)-7,8-dihydro- 1,6-naphthyridin-6(5H)- yl)nicotinonitrile | 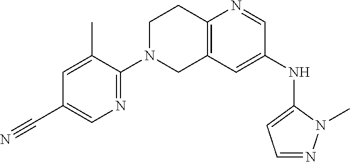 [SMILES: Cc1cc(C#N)cnc1N1CCc2ncc(Nc3ccnn3C)cc2C1] | 346.2 |
| 307 | 6-(3-(difluoromethyl)-7,8-dihydro- 1,6-naphthyridin-6(5H)-yl)-5- methylnicotinonitrile | 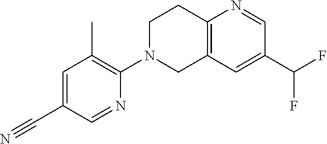 [SMILES: Cc1cc(C#N)cnc1N1CCc2ncc(C(F)F)cc2C1] | 301.2 |
| 308 | 6-(3-(difluoromethyl)-7,8-dihydro- 1,6-naphthyridin-6(5H)-yl)-4,5- dimethylpyridazine-3-carbonitrile | 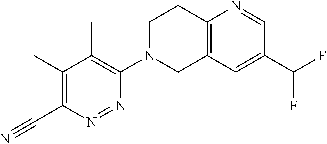 [SMILES: Cc1c(C#N)nnc(N2CCc3ncc(C(F)F)cc3C2)c1C] | 316.2 |
| 309 | 6-(3-(difluoromethyl)-7,8-dihydro- 1,6-naphthyridin-6(5H)-yl)-5- methylpyridazine-3-carbonitrile | 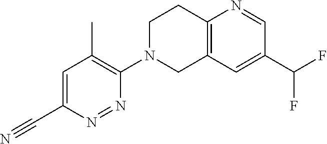 [SMILES: Cc1cc(C#N)nnc1N1CCc2ncc(C(F)F)cc2C1] | 302.2 |
| 310 | 6-(3-(2,4-difluorophenoxy)-7,8- dihydro-1,6-naphthyridin-6(5H)- yl)-5-methylnicotinonitrile | 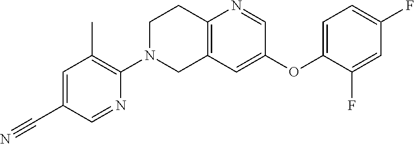 [SMILES: Cc1cc(C#N)cnc1N1CCc2ncc(Oc3ccc(F)cc3F)cc2C1] | 379.3 |
| 311 | 5-methyl-6-(3-((1-methyl-1H- pyrazol-5-yl)amino)-7,8-dihydro- 1,6-naphthyridin-6(5H)- yl)pyridazine-3-carbonitrile | 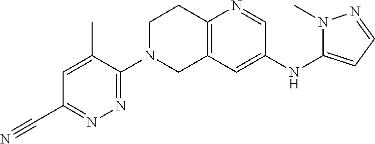 [SMILES: Cc1cc(C#N)nnc1N1CCc2ncc(Nc3ccnn3C)cc2C1] | 347.2 |
| 312 | 6-(3-(6-(dimethylamino)pyridin-3- yl)-7,8-dihydro-1,6-naphthyridin- 6(5H)-yl)-4,5-dimethylpyridazine- 3-carbonitrile | 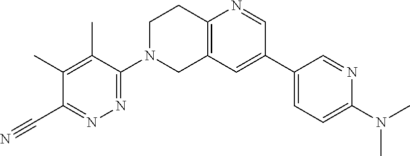 [SMILES: Cc1c(C#N)nnc(N2CCc3ncc(-c4ccc(N(C)C)nc4)cc3C2)c1C] | 386.2 |
| 313 | 6-(3-((1-isopentyl-3-methyl-1/- pyrazol-5-yl)amino)-7,8-dihydro- 1,6-naphthyridin-6(5H)-yl)-5- methylnicotinonitrile | 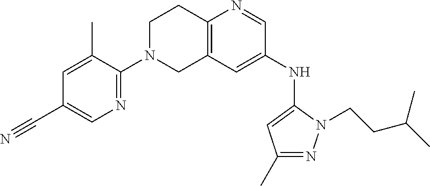 [SMILES: Cc1cc(Nc2cnc3c(c2)CN(c2ncc(C#N)cc2C)CC3)n(CCC(C)C)n1] | 416.5 |
| 314 | 6-(3-(6,7-dihydropyrazolo[1,5- a]pyrimidin-4(5H)-yl)-7,8-dihydro- 1,6-naphthyridin-6(5H)-yl)-5- methylnicotinonitrile | 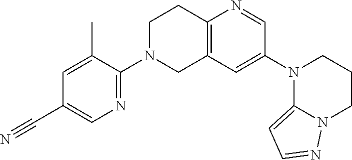 [SMILES: Cc1cc(C#N)cnc1N1CCc2ncc(N3CCCn4nccc43)cc2C1] | 372.4 |
| 315 | 6-(3-((1-butyl-3-methyl-1H- pyrazol-5-yl)amino)-7,8-dihydro- 1,6-naphthyridin-6(5H)-yl)-5- methylnicotinonitrile | 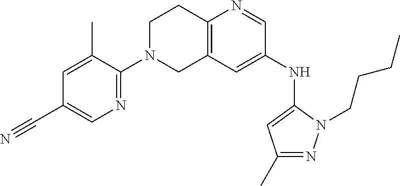 [SMILES: CCCCn1nc(C)cc1Nc1cnc2c(c1)CN(c1ncc(C#N)cc1C)CC2] | 402.4 |
| 316 | 6-(3-((1-isopropyl-3-methyl-1H- pyrazol-5-yl)amino)-7,8-dihydro- 1,6-naphthyridin-6(5H)-yl)-5- methylnicotinonitrile | 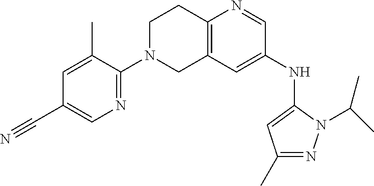 [SMILES: Cc1cc(Nc2cnc3c(c2)CN(c2ncc(C#N)cc2C)CC3)n(C(C)C)n1] | 388.4 |
| 317 | 6-(3-cyclopropyl-7,8-dihydro-1,6- naphthyridin-6(5H)-yl)-4,5- dimethylpyridazine-3-carbonitrile | 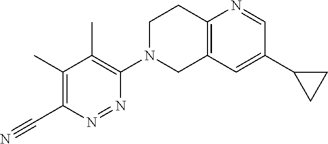 [SMILES: Cc1c(C#N)nnc(N2CCc3ncc(C4CC4)cc3C2)c1C] | 306.4 |
| 318 | 6-(3-(6-(dimethylamino)pyridin-3- yl)-7,8-dihydro-1,6-naphthyridin- 6(5H)-yl)-5-methylnicotinonitrile | 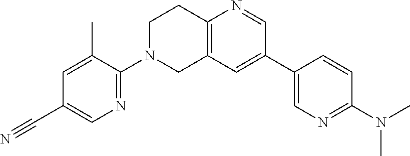 [SMILES: Cc1cc(C#N)cnc1N1CCc2ncc(-c3ccc(N(C)C)nc3)cc2C1] | 371.2 |
| 319 | 6-(3-(6-fluoropyridin-3-yl)-7,8- dihydro-1,6-naphthyridin-6(5H)- yl)-5-methylnicotinonitrile | 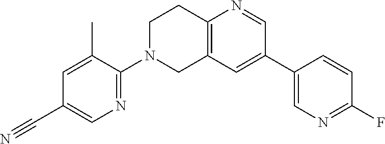 [SMILES: Cc1cc(C#N)cnc1N1CCc2ncc(-c3ccc(F)nc3)cc2C1] | 346.2 |
| 320 | 6-(3-(6-fluoro-4-methylpyridin-3- yl)-7,8-dihydro-1,6-naphthyridin- 6(5H)-yl)-4,5-dimethylpyridazine- 3-carbonitrile | 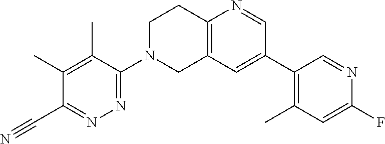 [SMILES: Cc1cc(F)ncc1-c1cnc2c(c1)CN(c1nnc(C#N)c(C)c1C)CC2] | 375.2 |
| 321 | 6-(3-(6-cyclopropylpyridin-3-yl)- 7,8-dihydro-1,6-naphthyridin- 6(5H)-yl)-5-methylpyridazine-3- carbonitrile | 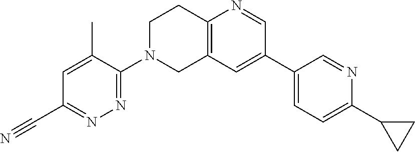 [SMILES: Cc1cc(C#N)nnc1N1CCc2ncc(-c3ccc(C4CC4)nc3)cc2C1] | 369.2 |
| 322 | 5-methyl-6-(3-(4-methylpyridin-3- yl)-7,8-dihydro-1,6-naphthyridin- 6(5H)-yl)pyridazine-3-carbonitrile | 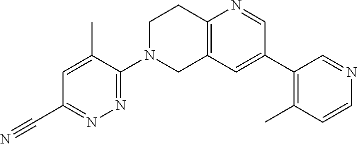 [SMILES: Cc1ccncc1-c1cnc2c(c1)CN(c1nnc(C#N)cc1C)CC2] | 343.2 |
| 323 | 6-(3-(1,3-dimethyl-1H-pyrazol-4- yl)-7,8-dihydro-1,6-naphthyridin- 6(5H)-yl)-5-methylpyridazine-3- carbonitrile | 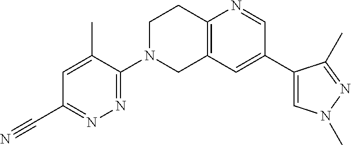 [SMILES: Cc1cc(C#N)nnc1N1CCc2ncc(-c3cn(C)nc3C)cc2C1] | 346.2 |
| 324 | 6-(3-(1-(2-(dimethylamino)ethyl)- 1H-pyrazol-4-yl)-7,8-dihydro-1,6- naphthyridin-6(5H)-yl)-4,5- dimethylpyridazine-3-carbonitrile | 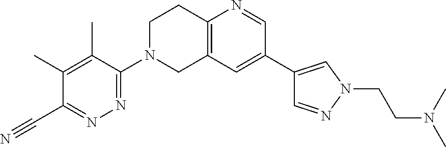 [SMILES: Cc1c(C#N)nnc(N2CCc3ncc(-c4cnn(CCN(C)C)c4)cc3C2)c1C] | 403.2 |
| 325 | 4,5-dimethyl-6-(3-(1-(2- morpholinoethyl)-1H-pyrazol-4- yl)-7,8-dihydro-1,6-naphthyridin- 6(5H)-yl)pyridazine-3-carbonitrile | 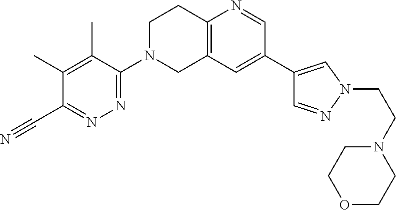 [SMILES: Cc1c(C#N)nnc(N2CCc3ncc(-c4cnn(CCN5CCOCC5)c4)cc3C2)c1C] | 445.2 |
| 326 | 6-(3-(1-(difluoromethyl)-1H- pyrazol-4-yl)-7,8-dihydro-1,6- naphthyridin-6(5H)-yl)-4,5- dimethylpyridazine-3-carbonitrile | 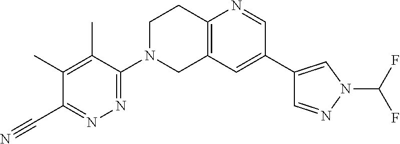 [SMILES: Cc1c(C#N)nnc(N2CCc3ncc(-c4cnn(C(F)F)c4)cc3C2)c1C] | 382.2 |
| 327 | 6-(3-(1-(2-hydroxy-2- methylpropyl)-1H-pyrazol-4-yl)- 7,8-dihydro-1,6-naphthyridin- 6(5H)-yl)-4,5-dimethylpyridazine- 3-carbonitrile | 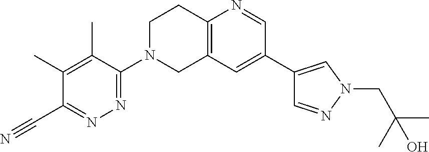 [SMILES: Cc1c(C#N)nnc(N2CCc3ncc(-c4cnn(CC(C)(C)O)c4)cc3C2)c1C] | 404.2 |
| 328 | 4,5-dimethyl-6-(3-(6-(4- methylpiperazin-1-yl)pyridin-3-yl)- 7,8-dihydro-1,6-naphthyridin- 6(5H)-yl)pyridazine-3-carbonitrile | 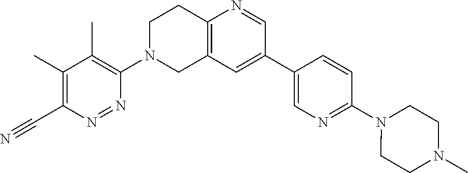 [SMILES: Cc1c(C#N)nnc(N2CCc3ncc(-c4ccc(N5CCN(C)CC5)nc4)cc3C2)c1C] | 441.2 |
| 329 | 6-(3-(6-(3-(2-hydroxypropan-2- yl)azetidin-1-yl)pyridin-3-yl)-7,8- dihydro-1,6-naphthyridin-6(5H)- yl)-4,5-dimethylpyridazine-3- carbonitrile | 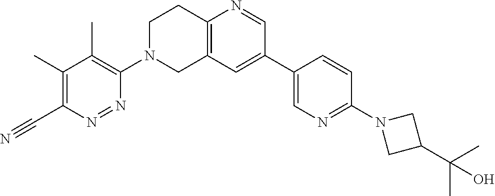 [SMILES: Cc1c(C#N)nnc(N2CCc3ncc(-c4ccc(N5CC(C(C)(C)O)C5)nc4)cc3C2)c1C] | 456.2 |
| 330 | 6-(3-cyclopropyl-7,8-dihydro-1,6- naphthyridin-6(5H)-yl)-5- methylnicotinonitrile | 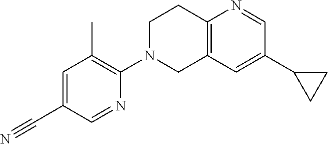 [SMILES: Cc1cc(C#N)cnc1N1CCc2ncc(C3CC3)cc2C1] | 291.4 |
| 331 | 5-methyl-6-(3-(methyl(1-methyl- 1H-pyrazol-5-yl)amino)-7,8- dihydro-1,6-naphthyridin-6(5H)- yl)nicotinonitrile | 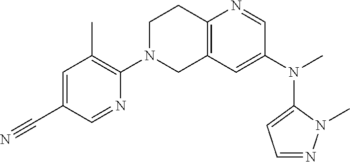 [SMILES: Cc1cc(C#N)cnc1N1CCc2ncc(N(C)c3ccnn3C)cc2C1] | 360.4 |
| 332 | 6-(3-(6-(2-oxa-6- azaspiro[3.3]heptan-6-yl)pyridin-3- yl)-7,8-dihydro-1,6-naphthyridin- 6(5H)-yl)-4,5-dimethylpyridazine- 3-carbonitrile | 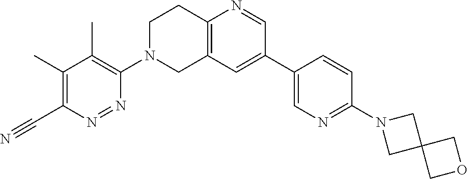 [SMILES: Cc1c(C#N)nnc(N2CCc3ncc(-c4ccc(N5CC6(COC6)C5)nc4)cc3C2)c1C] | 440.2 |
| 333 | 6-(3-(6-(2-oxa-5- azabicyclo[2.2.1]heptan-5- yl)pyridin-3-yl)-7,8-dihydro-1,6- naphthyridin-6(5H)-yl)-4,5- dimethylpyridazine-3-carbonitrile | 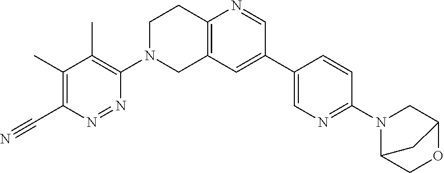 [SMILES: Cc1c(C#N)nnc(N2CCc3ncc(-c4ccc(N5CC6CC5CO6)nc4)cc3C2)c1C] | 440.2 |
| 334 | 6-(3-(6-fluoropyridin-3-yl)-7,8- dihydro-1,6-naphthyridin-6(5H)- yl)-4,5-dimethylpyridazine-3- carbonitrile | 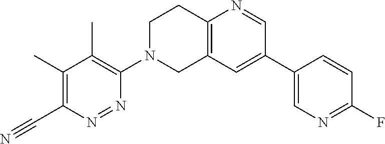 [SMILES: Cc1c(C#N)nnc(N2CCc3ncc(-c4ccc(F)nc4)cc3C2)c1C] | 361.2 |
| 335 | 6-(3-(6-(4-cyclopropylpiperazin-1- yl)pyridin-3-yl)-7,8-dihydro-1,6- naphthyridin-6(5H)-yl)-4,5- dimethylpyridazine-3-carbonitrile | 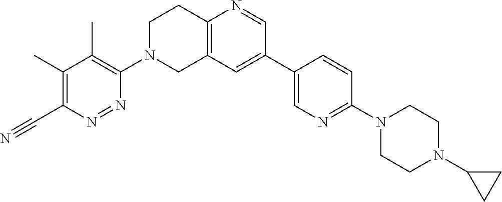 [SMILES: Cc1c(C#N)nnc(N2CCc3ncc(-c4ccc(N5CCN(C6CC6)CC5)nc4)cc3C2)c1C] | 467.2 |
| 336 | 4-(6-(5-cyano-3-methylpyridin-2- yl)-5,6,7,8-tetrahydro-1,6- naphthyridin-3-yl)picolinonitrile | 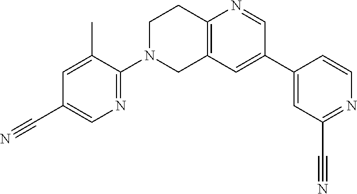 [SMILES: Cc1cc(C#N)cnc1N1CCc2ncc(-c3ccnc(C#N)c3)cc2C1] | 353.3 |
| 337 | 6-(3-(2,5-difluoropyridin-4-yl)-7,8- dihydro-1,6-naphthyridin-6(5H)- yl)-5-methylnicotinonitrile | 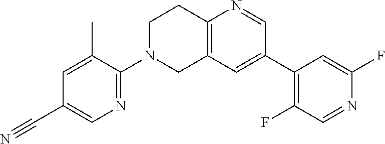 [SMILES: Cc1cc(C#N)cnc1N1CCc2ncc(-c3cc(F)ncc3F)cc2C1] | 364.3 |
| 338 | 6-(3-(2-methoxypyridin-4-yl)-7,8- dihydro-1,6-naphthyridin-6(5H)- yl)-5-methylnicotinonitrile | 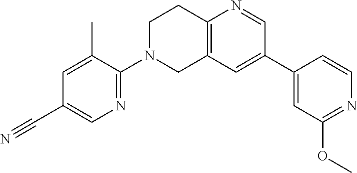 [SMILES: COc1cc(-c2cnc3c(c2)CN(c2ncc(C#N)cc2C)CC3)ccn1] | 358.2 |
| 339 | 6-(3-(2-cyclopropylpyridin-4-yl)- 7,8-dihydro-1,6-naphthyridin- 6(5H)-yl)-5-methylnicotinonitrile | 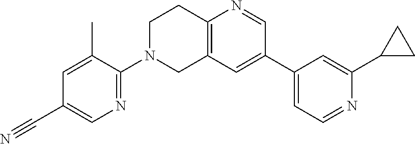 [SMILES: Cc1cc(C#N)cnc1N1CCc2ncc(-c3ccnc(C4CC4)c3)cc2C1] | 368.4 |
| 340 | 6-(3-(2-fluoropyridin-4-yl)-7,8- dihydro-1,6-naphthyridin-6(5)- yl)-5-methylnicotinonitrile | 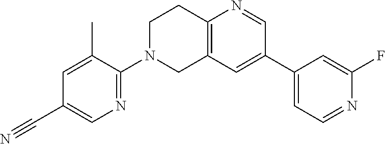 [SMILES: Cc1cc(C#N)cnc1N1CCc2ncc(-c3ccnc(F)c3)cc2C1] | 346.2 |
| 341 | 6-(3-(5-chloro-2-methoxypyridin-4- yl)-7,8-dihydro-1,6-naphthyridin- 6(5H)-yl)-5-methylnicotinonitrile | 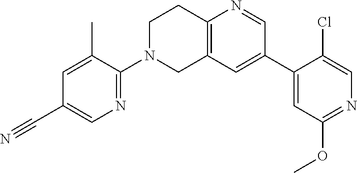 [SMILES: COc1cc(-c2cnc3c(c2)CN(c2ncc(C#N)cc2C)CC3)c(Cl)cn1] | 392.2 |
| 342 | 6-(3-(6,7-dihydropyrazolo[1,5- a]pyrimidin-4(5H)-yl)-7,8-dihydro- 1,6-naphthyridin-6(5H)-yl)-4,5- dimethylpyridazine-3-carbonitrile | 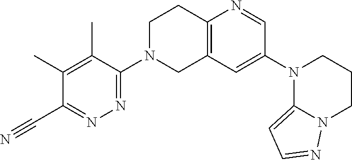 [SMILES: Cc1c(C#N)nnc(N2CCc3ncc(N4CCCn5nccc54)cc3C2)c1C] | 387.3 |
| 343 | 6-(3-(1H-imidazol-1-yl)-7,8- dihydro-1,6-naphthyridin-6(5H)- yl)-5-methylnicotinonitrile | 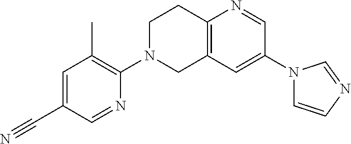 [SMILES: Cc1cc(C#N)cnc1N1CCc2ncc(-n3ccnc3)cc2C1] | 317.3 |
| 344 | 6-(3-(1H-pyrazol-1-yl)-7,8- dihydro-1,6-naphthyridin-6(5H)- yl)-5-methylnicotinonitrile | 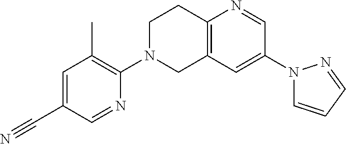 [SMILES: Cc1cc(C#N)cnc1N1CCc2ncc(-n3cccn3)cc2C1] | 317.3 |
| 345 | 5-methyl-6-(3-(5-methyl-1H- pyrazol-1-yl)-7,8-dihydro-1,6- naphthyridin-6(5H)- yl)nicotinonitrile | 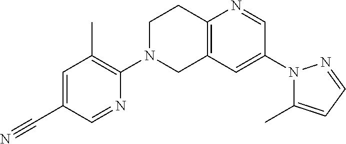 [SMILES: Cc1cc(C#N)cnc1N1CCc2ncc(-n3nccc3C)cc2C1] | 331.3 |
| 346 | 6-(3-(3,5-dimethyl-1H-pyrazol-1- yl)-7,8-dihydro-1,6-naphthyridin- 6(5H)-yl)-5-methylnicotinonitrile | 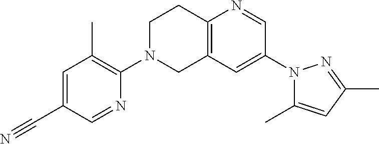 [SMILES: Cc1cc(C)n(-c2cnc3c(c2)CN(c2ncc(C#N)cc2C)CC3)n1] | 345.4 |
| 347 | 5-methyl-6-(8-methyl-3- (trifluoromethyl)-7,8-dihydro-1,6- naphthyridin-6(5H)- yl)nicotinonitrile | 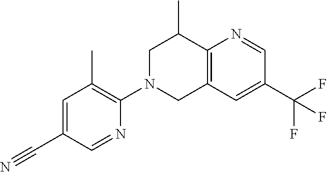 [SMILES: Cc1cc(C#N)cnc1N1Cc2cc(C(F)(F)F)cnc2C(C)C1] | 333.3 |
| 348 | 4,5-dimethyl-6-(8-methyl-3- (trifluoromethyl)-7,8-dihydro-1,6- naphthyridin-6(5H)-yl)pyridazine- 3-carbonitrile | 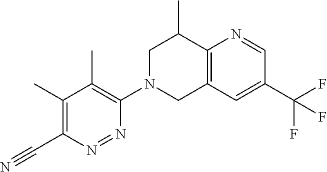 [SMILES: Cc1c(C#N)nnc(N2Cc3cc(C(F)(F)F)cnc3C(C)C2)c1C] | 348.3 |
| 349 | 6-(3-(2-fluoro-4-methylphenoxy)- 7,8-dihydro-1,6-naphthyridin- 6(5H)-yl)-5-methylnicotinonitrile | 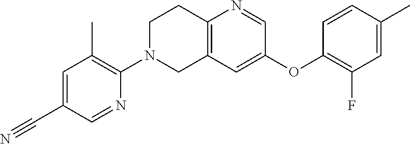 [SMILES: Cc1ccc(Oc2cnc3c(c2)CN(c2ncc(C#N)cc2C)CC3)c(F)c1] | 375.4 |
| 350 | 6-(3-(2,3-difluorophenoxy)-7,8- dihydro-1,6-naphthyridin-6(5H)- yl)-5-methylnicotinonitrile | 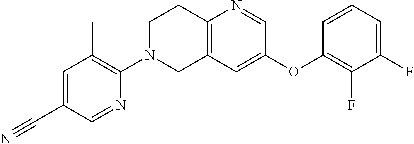 [SMILES: Cc1cc(C#N)cnc1N1CCc2ncc(Oc3cccc(F)c3F)cc2C1] | 379.3 |
| 351 | 6-(3-(2,5-difluorophenoxy)-7,8- dihydro-1,6-naphthyridin-6(5H)- yl)-5-methylnicotinonitrile | 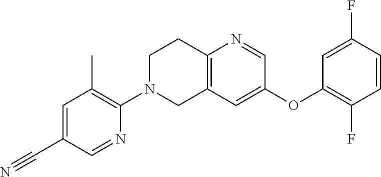 [SMILES: Cc1cc(C#N)cnc1N1CCc2ncc(Oc3cc(F)ccc3F)cc2C1] | 379.3 |
| 352 | 6-(3-(2,3-difluorophenoxy)-7,8- dihydro-1,6-naphthyridin-6(5H)- yl)-4,5-dimethylpyridazine-3- carbonitrile | 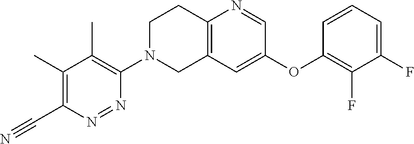 [SMILES: Cc1c(C#N)nnc(N2CCc3ncc(Oc4cccc(F)c4F)cc3C2)c1C] | 394.3 |
| 353 | 6-(3-(2,5-difluorophenoxy)-7,8- dihydro-1,6-naphthyridin-6(5H)- yl)-4,5-dimethylpyridazine-3- carbonitrile | 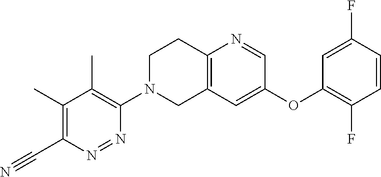 [SMILES: Cc1c(C#N)nnc(N2CCc3ncc(Oc4cc(F)ccc4F)cc3C2)c1C] | 394.4 |
| 354 | 6-(3-(2,4-difluorophenoxy)-7,8- dihydro-1,6-naphthyridin-6(5H)- yl)-4,5-dimethylpyridazine-3- carbonitrile | 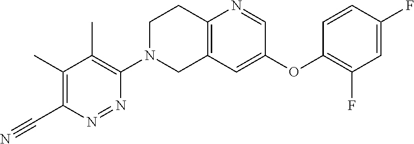 [SMILES: Cc1c(C#N)nnc(N2CCc3ncc(Oc4ccc(F)cc4F)cc3C2)c1C] | 394 |
| 355 | 6-(3-(2,4-difluorophenoxy)-7,8- dihydro-1,6-naphthyridin-6(5H)- yl)-5-methylpyridazine-3- carbonitrile | 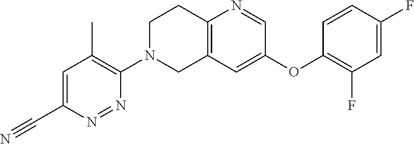 [SMILES: Cc1cc(C#N)nnc1N1CCc2ncc(Oc3ccc(F)cc3F)cc2C1] | 380 |
| 356 | 6-(3-(2,3-difluorophenoxy)-7,8- dihydro-1,6-naphthyridin-6(5H)- yl)-5-methylpyridazine-3- carbonitrile | 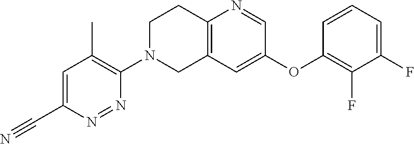 [SMILES: Cc1cc(C#N)nnc1N1CCc2ncc(Oc3cccc(F)c3F)cc2C1] | 380 |
| 357 | 6-(3-(2,5-difluorophenoxy)-7,8- dihydro-1,6-naphthyridin-6(5H)- yl)-5-methylpyridazine-3- carbonitrile | 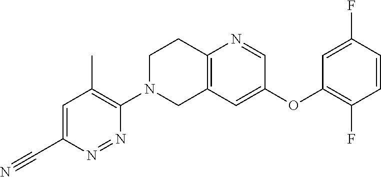 [SMILES: Cc1cc(C#N)nnc1N1CCc2ncc(Oc3cc(F)ccc3F)cc2C1] | 380 |
| 358 | 6-(3-(2-fluoro-4-methylphenoxy)- 7,8-dihydro-1,6-naphthyridin- 6(5H)-yl)-4,5-dimethylpyridazine- 3-carbonitrile | 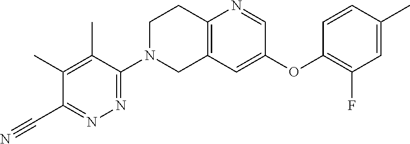 [SMILES: Cc1ccc(Oc2cnc3c(c2)CN(c2nnc(C#N)c(C)c2C)CC3)c(F)c1] | 390 |
| 359 | 5-methyl-6-(3-(7-(trifluoromethyl)- 6,7-dihydropyrazolo[1,5- a]pyrimidin-4(5H)-yl)-7,8-dihydro- 1,6-naphthyridin-6(5H)- yl)nicotinonitrile | 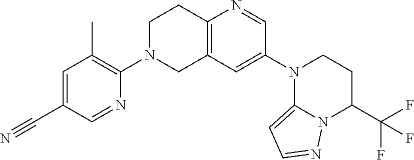 [SMILES: Cc1cc(C#N)cnc1N1CCc2ncc(N3CCC(C(F)(F)F)n4nccc43)cc2C1] | 440.4 |
| 360 | 6-(3-(6,7-dihydropyrazolo[1,5- a]pyrimidin-4(5H)-yl)-7,8-dihydro- 1,6-naphthyridin-6(5H)-yl)-5- methylpyridazine-3-carbonitrile | 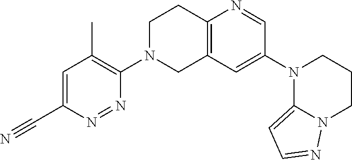 [SMILES: Cc1cc(C#N)nnc1N1CCc2ncc(N3CCCn4nccc43)cc2C1] | 373.3 |
| 361 | 6-(3-(1-cyclopropyl-1H-pyrazol-4- yl)-7,8-dihydro-1,6-naphthyridin- 6(5H)-yl)-5-methylpyridazine-3- carbonitrile | 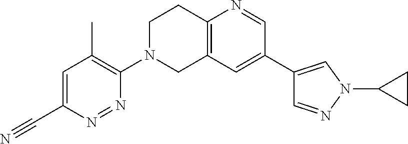 [SMILES: Cc1cc(C#N)nnc1N1CCc2ncc(-c3cnn(C4CC4)c3)cc2C1] | 358.4 |
| 362 | 6-(3-(3,4-dihydro-1,5-naphthyridin- 1(2H)-yl)-7,8-dihydro-1,6- naphthyridin-6(5H)-yl)-5- methylnicotinonitrile | 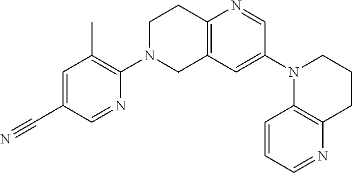 [SMILES: Cc1cc(C#N)cnc1N1CCc2ncc(N3CCCc4ncccc43)cc2C1] | 383.4 |
| 363 | 4,5-dimethyl-6-(3-(2-methyl-6,7- dihydropyrazolo[1,5-a]pyrimidin- 4(5H)-yl)-7,8-dihydro-1,6- naphthyridin-6(5H)-yl)pyridazine- 3-carbonitrile | 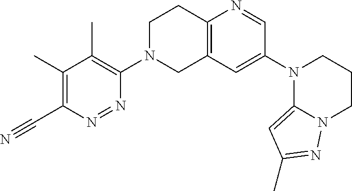 [SMILES: Cc1cc2n(n1)CCCN2c1cnc2c(c1)CN(c1nnc(C#N)c(C)c1C)CC2] | 401 |
| 364 | 4,5-dimethyl-6-(3-(7-methyl-6,7- dihydropyrazolo[1,5-a]pyrimidin- 4(5H)-yl)-7,8-dihydro-1,6- naphthyridin-6(5H)-yl)pyridazine- 3-carbonitrile | 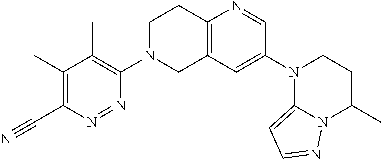 [SMILES: Cc1c(C#N)nnc(N2CCc3ncc(N4CCC(C)n5nccc54)cc3C2)c1C] | 401 |
| 365 | 6-(3-(2-fluorobenzyl)-7,8-dihydro- 1,6-naphthyridin-6(5H)-yl)-5- methylnicotinonitrile | 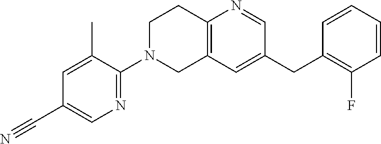 [SMILES: Cc1cc(C#N)cnc1N1CCc2ncc(Cc3ccccc3F)cc2C1] | 359 |
| 366 | 5-methyl-6-(7-methyl-3- (trifluoromethyl)-7,8-dihydro-1,6- naphthyridin-6(5H)- yl)nicotinonitrile | 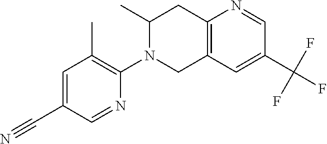 [SMILES: Cc1cc(C#N)cnc1N1Cc2cc(C(F)(F)F)cnc2CC1C] | 333.3 |
| 367 | 4,5-dimethyl-6-(7-methyl-3- (trifluoromethyl)-7,8-dihydro-1,6- naphthyridin-6(5H)-yl)pyridazine- 3-carbonitrile | 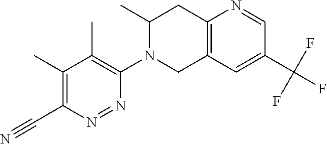 [SMILES: Cc1c(C#N)nnc(N2Cc3cc(C(F)(F)F)cnc3CC2C)c1C] | 348.3 |
| 368 | 6-(3-(2-fluoro-5-methylphenoxy)- 7,8-dihydro-1,6-naphthyridin- 6(5H)-yl)-5-methylnicotinonitrile | 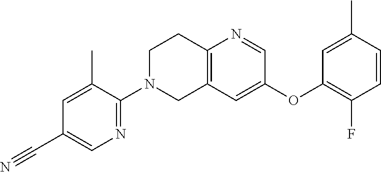 [SMILES: Cc1ccc(F)c(Oc2cnc3c(c2)CN(c2ncc(C#N)cc2C)CC3)c1] | 375 |
| 369 | 6-(3-(2-fluorobenzyl)-7,8-dihydro- 1,6-naphthyridin-6(5H)-yl)-4,5- dimethylpyridazine-3-carbonitrile | 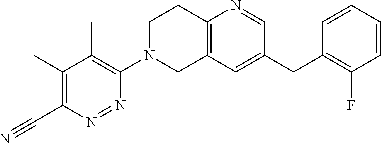 [SMILES: Cc1c(C#N)nnc(N2CCc3ncc(Cc4ccccc4F)cc3C2)c1C] | 374 |
| 370 | 6-(3-(2,2-dimethylmorpholino)-7,8- dihydro-1,6-naphthyridin-6(5H)- yl)-5-methylnicotinonitrile | 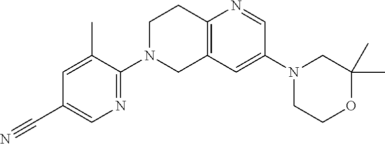 [SMILES: Cc1cc(C#N)cnc1N1CCc2ncc(N3CCOC(C)(C)C3)cc2C1] | 364 |
| 371 | 2-methoxy-5-methyl-6-(3- (trifluoromethyl)-7,8-dihydro-1,6- naphthyridin-6(5H)- yl)nicotinonitrile | 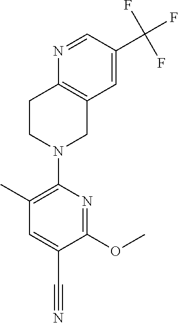 [SMILES: COc1nc(N2CCc3ncc(C(F)(F)F)cc3C2)c(C)cc1C#N] | 349.1 |
| 372 | 5-methyl-2-(1-methyl-1H-pyrrol-2- yl)-6-(3-(trifluoromethyl)-7,8- dihydro-1,6-naphthyridin-6(5H)- yl)nicotinonitrile | 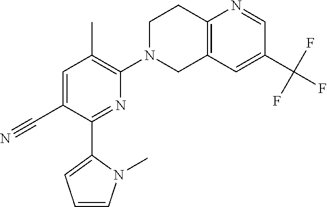 [SMILES: Cc1cc(C#N)c(-c2cccn2C)nc1N1CCc2ncc(C(F)(F)F)cc2C1] | 398.4 |
| 373 | 2-ethoxy-5-methyl-6-(3- (trifluoromethyl)-7,8-dihydro-1,6- naphthyridin-6(5H)- yl)nicotinonitrile | 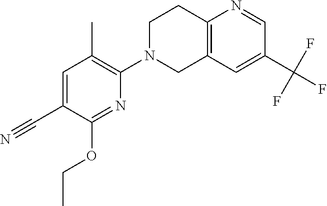 [SMILES: CCOc1nc(N2CCc3ncc(C(F)(F)F)cc3C2)c(C)cc1C#N] | 363.4 |
| 374 | 4,5-dimethyl-6-(3-(6-methyl-6,7- dihydropyrazolo[1,5-a]pyrimidin- 4(5H)-yl)-7,8-dihydro-1,6- naphthyridin-6(5H)-yl)pyridazine- 3-carbonitrile | 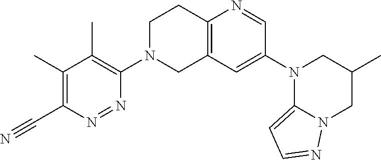 [SMILES: Cc1c(C#N)nnc(N2CCc3ncc(N4CC(C)Cn5nccc54)cc3C2)c1C] | 401 |
| 375 | (R)-5-methyl-6-(3-(2- phenylmorpholino)-7,8-dihydro- 1,6-naphthyridin-6(5H)- yl)nicotinonitrile | 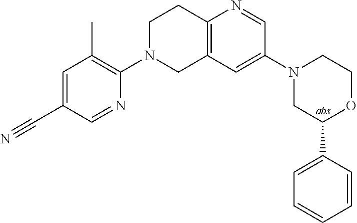 [SMILES: Cc1cc(C#N)cnc1N1CCc2ncc(N3CCO[C@H](c4ccccc4)C3)cc2C1] | 412 |
| 376 | (R)-4,5-dimethyl-6-(3-(2- phenylmorpholino)-7,8-dihydro- 1,6-naphthyridin-6(5H)- yl)pyridazine-3-carbonitrile | 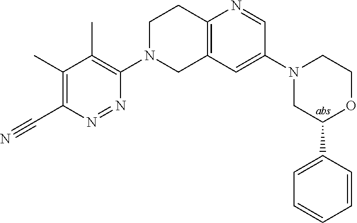 [SMILES: Cc1c(C#N)nnc(N2CCc3ncc(N4CCO[C@H](c5ccccc5)C4)cc3C2)c1C] | 427 |
| 377 | 6-(3-(2,2-dimethylmorpholino)-2- methyl-7,8-dihydro-1,6- naphthyridin-6(5H)-yl)-5- methylnicotinonitrile | 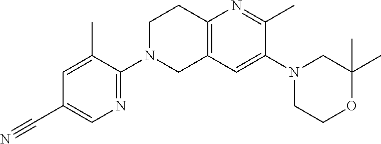 [SMILES: Cc1cc(C#N)cnc1N1CCc2nc(C)c(N3CCOC(C)(C)C3)cc2C1] | 378 |
| 378 | (S)-5-methyl-6-(3-(2- phenylmorpholino)-7,8-dihydro- 1,6-naphthyridin-6(5H)- yl)nicotinonitrile | 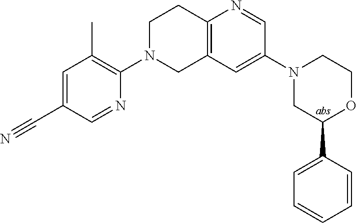 [SMILES: Cc1cc(C#N)cnc1N1CCc2ncc(N3CCO[C@@H](c4ccccc4)C3)cc2C1] | 412 |
| 379 | (S)-4,5-dimethyl-6-(3-(2- phenylmorpholino)-7,8-dihydro- 1,6-naphthyridin-6(5H)- yl)pyridazine-3-carbonitrile | 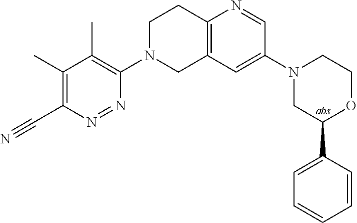 [SMILES: Cc1c(C#N)nnc(N2CCc3ncc(N4CCO[C@@H](c5ccccc5)C4)cc3C2)c1C] | 427 |
| 380 | 4,5-dimethyl-6-(3-(2-(pyridin-2- yl)morpholino)-7,8-dihydro-1,6- naphthyridin-6(5H)-yl)pyridazine- 3-carbonitrile | 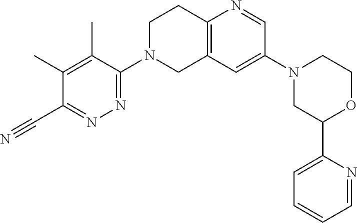 [SMILES: Cc1c(C#N)nnc(N2CCc3ncc(N4CCOC(c5ccccn5)C4)cc3C2)c1C] | 428 |
| 381 | 5-methyl-6-(3-(6-methyl-6,7- dihydropyrazolo[1,5-a]pyrimidin- 4(5H)-yl)-7,8-dihydro-1,6- naphthyridin-6(5H)-yl)pyridazine- 3-carbonitrile | 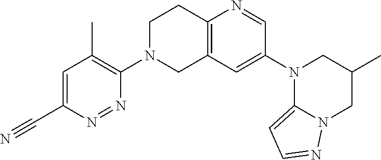 [SMILES: Cc1cc(C#N)nnc1N1CCc2ncc(N3CC(C)Cn4nccc43)cc2C1] | 387 |
| 382 | 5-methyl-6-(3-(2-methyl-6,7- dihydropyrazolo[1,5-a]pyrimidin- 4(5H)-yl)-7,8-dihydro-1,6- naphthyridin-6(5H)-yl)pyridazine- 3-carbonitrile | 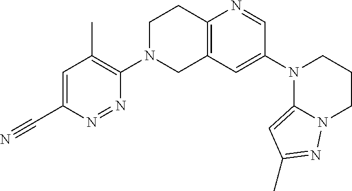 [SMILES: Cc1cc2n(n1)CCCN2c1cnc2c(c1)CN(c1nnc(C#N)cc1C)CC2] | 387 |
| 383 | 5-methyl-6-(3-(7-methyl-6,7- dihydropyrazolo[1,5-a]pyrimidin- 4(5H)-yl)-7,8-dihydro-1,6- naphthyridin-6(5H)-yl)pyridazine- 3-carbonitrile | 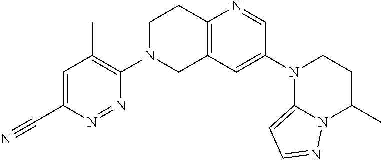 [SMILES: Cc1cc(C#N)nnc1N1CCc2ncc(N3CCC(C)n4nccc43)cc2C1] | 387 |
| 384 | 5-methyl-6-(3-(6-methyl-6,7- dihydropyrazolo[1,5-a]pyrimidin- 4(5H)-yl)-7,8-dihydro-1,6- naphthyridin-6(5H)- yl)nicotinonitrile | 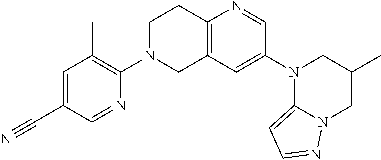 [SMILES: Cc1cc(C#N)cnc1N1CCc2ncc(N3CC(C)Cn4nccc43)cc2C1] | 386 |
| 385 | 5-methyl-6-(3-(2-methyl-6,7- dihydropyrazolo[1,5-a]pyrimidin- 4(5H)-yl)-7,8-dihydro-1,6- naphthyridin-6(5H)- yl)nicotinonitrile | 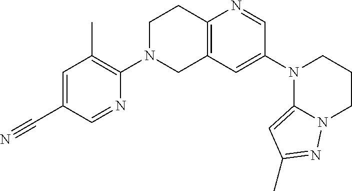 [SMILES: Cc1cc2n(n1)CCCN2c1cnc2c(c1)CN(c1ncc(C#N)cc1C)CC2] | 386 |
| 386 | 5-methyl-6-(3-(7-methyl-6,7- dihydropyrazolo[1,5-a]pyrimidin- 4(5H)-yl)-7,8-dihydro-1,6- naphthyridin-6(5H)- yl)nicotinonitrile | 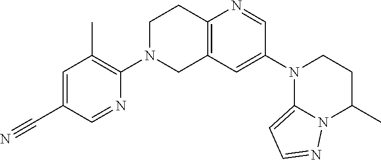 [SMILES: Cc1cc(C#N)cnc1N1CCc2ncc(N3CCC(C)n4nccc43)cc2C1] | 386 |
| 387 | 5-methyl-6-(3-(2-(pyridin-2- yl)morpholino)-7,8-dihydro-1,6- naphthyridin-6(5H)- yl)nicotinonitrile | 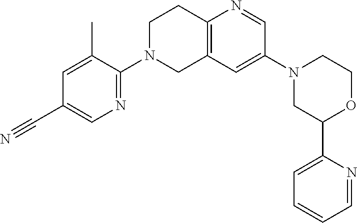 [SMILES: Cc1cc(C#N)cnc1N1CCc2ncc(N3CCOC(c4ccccn4)C3)cc2C1] | 413 |
| 388 | 4,5-dimethyl-6-(3-(2-(pyridin-3- yl)morpholino)-7,8-dihydro-1,6- naphthyridin-6(5H)-yl)pyridazine- 3-carbonitrile | 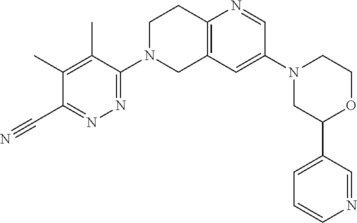 [SMILES: Cc1c(C#N)nnc(N2CCc3ncc(N4CCOC(c5cccnc5)C4)cc3C2)c1C] | 428 |
| 389 | 5-methyl-6-(3-(2-(pyridin-3- yl)morpholino)-7,8-dihydro-1,6- naphthyridin-6(5H)- yl)nicotinonitrile | 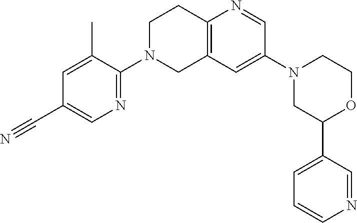 [SMILES: Cc1cc(C#N)cnc1N1CCc2ncc(N3CCOC(c4cccnc4)C3)cc2C1] | 413 |
| 390 | 5-methyl-6-(3-(2-methylthiazol-5- yl)-7,8-dihydro-1,6-naphthyridin- 6(5H)-yl)pyridazine-3-carbonitrile | 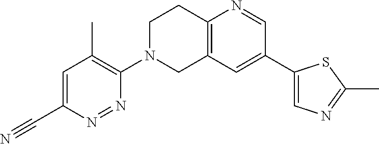 [SMILES: Cc1ncc(-c2cnc3c(c2)CN(c2nnc(C#N)cc2C)CC3)s1] | 349 |
| 391 | 6-(3-(2,3-dihydro-1H-pyrido[2,3- b][1,4]oxazin-1-yl)-7,8-dihydro- 1,6-naphthyridin-6(5H)-yl)-5- methylpyridazine-3-carbonitrile | 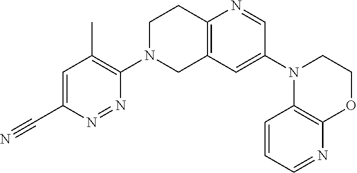 [SMILES: Cc1cc(C#N)nnc1N1CCc2ncc(N3CCOc4ncccc43)cc2C1] | 386.5 |
| 392 | 5-methyl-6-(3-(6-methylpyridin-3- yl)-7,8-dihydro-1,6-naphthyridin- 6(5H)-yl)pyridazine-3-carbonitrile | 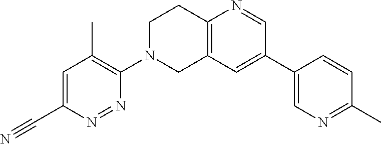 [SMILES: Cc1ccc(-c2cnc3c(c2)CN(c2nnc(C#N)cc2C)CC3)cn1] | 343.4 |
| 393 | 6-(3-cyclopropyl-7,8-dihydro-1,6- naphthyridin-6(5H)-yl)-5- methylpyridazine-3-carbonitrile | 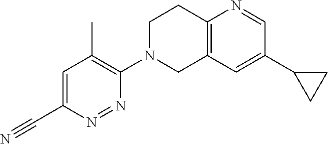 [SMILES: Cc1cc(C#N)nnc1N1CCc2ncc(C3CC3)cc2C1] | 292.4 |
| 394 | 5-methyl-6-(3-(1-methyl-1H- pyrazol-5-yl)-7,8-dihydro-1,6- naphthyridin-6(5H)-yl)pyridazine- 3-carbonitrile | 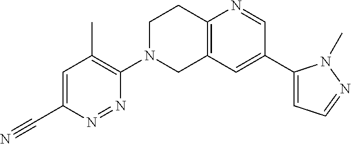 [SMILES: Cc1cc(C#N)nnc1N1CCc2ncc(-c3ccnn3C)cc2C1] | 332.4 |
| 395 | 5-methyl-6-(3-(tetrahydrofuran-3- yl)-7,8-dihydro-1,6-naphthyridin- 6(5H)-yl)nicotinonitrile | 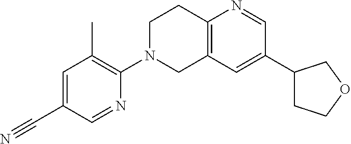 [SMILES: Cc1cc(C#N)cnc1N1CCc2ncc(C3CCOC3)cc2C1] | 321.4 |
| 396 | 6-(3-(2-fluorophenoxy)-7,8- dihydro-1,6-naphthyridin-6(5H)- yl)-5-methylpyridazine-3- carbonitrile | 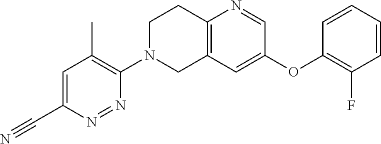 [SMILES: Cc1cc(C#N)nnc1N1CCc2ncc(Oc3ccccc3F)cc2C1] | 362.4 |
| 397 | (R)-5-methyl-6-(3-(2- methylmorpholino)-7,8-dihydro- 1,6-naphthyridin-6(5H)- yl)pyridazine-3-carbonitrile | 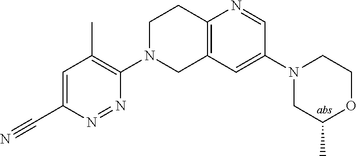 [SMILES: Cc1cc(C#N)nnc1N1CCc2ncc(N3CCO[C@H](C)C3)cc2C1] | 351.4 |
| 398 | 6-(3-(2,2-dimethylmorpholino)-7,8- dihydro-1,6-naphthyridin-6(5H)- yl)-5-methylpyridazine-3- carbonitrile | 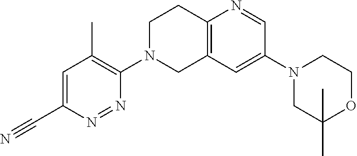 | 365.4 |
| 399 | 6-(3-((2-fluoropyridin-3-yl)amino)- 7,8-dihydro-1,6-naphthyridin- 6(5H)-yl)-5-methylpyridazine-3- carbonitrile | 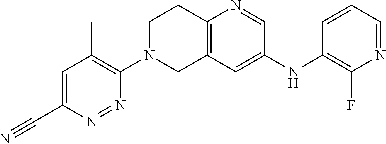 [SMILES: Cc1cc(C#N)nnc1N1CCc2ncc(Nc3cccnc3F)cc2C1] | 362.3 |
| 400 | 6-(3-(1H-pyrazol-1-yl)-7,8- dihydro-1,6-naphthyridin-6(5H)- yl)-5-methylpyridazine-3- carbonitrile | 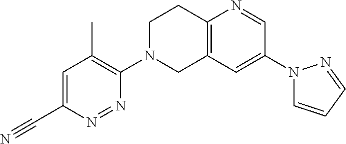 [SMILES: Cc1cc(C#N)nnc1N1CCc2ncc(-n3cccn3)cc2C1] | 318.3 |
| 401 | 6-(3-(3,5-dimethylisoxazol-4-yl)- 7,8-dihydro-1,6-naphthyridin- 6(5H)-yl)-5-methylpyridazine-3- carbonitrile | 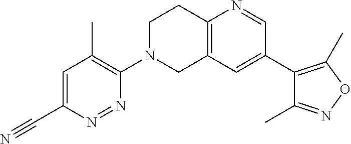 [SMILES: Cc1cc(C#N)nnc1N1CCc2ncc(-c3c(C)noc3C)cc2C1] | 347.3 |
| 402 | 6-(3-cyclobutyl-7,8-dihydro-1,6- naphthyridin-6(5H)-yl)-5- methylpyridazine-3-carbonitrile | 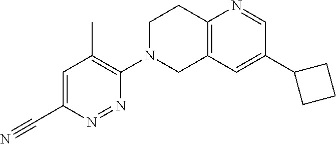 [SMILES: Cc1cc(C#N)nnc1N1CCc2ncc(C3CCC3)cc2C1] | 306.4 |
| 403 | 6-(3-cyclobutyl-7,8-dihydro-1,6- naphthyridin-6(5H)-yl)-5- methylnicotinonitrile | 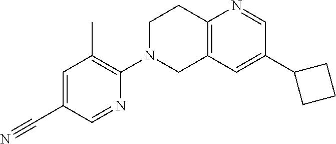 [SMILES: Cc1cc(C#N)cnc1N1CCc2ncc(C3CCC3)cc2C1] | 305.4 |
| 404 | 5-methyl-6-(8-methyl-3- morpholino-7,8-dihydro-1,6- naphthyridin-6(5H)-yl)pyridazine- 3-carbonitrile | 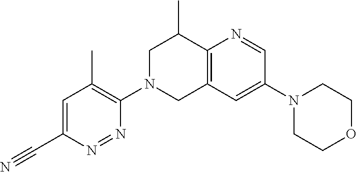 [SMILES: Cc1cc(C#N)nnc1N1Cc2cc(N3CCOCC3)cnc2C(C)C1] | 351 |
| 405 | 5-methyl-6-(8-methyl-3- morpholino-7,8-dihydro-1,6- naphthyridin-6(5H)- yl)nicotinonitrile | 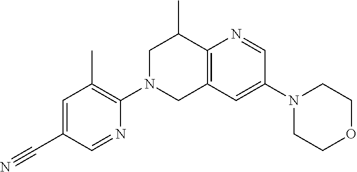 [SMILES: Cc1cc(C#N)cnc1N1Cc2cc(N3CCOCC3)cnc2C(C)C1] | 350.2 |
| 406^{a} | 5-methyl-6-(8-methyl-3- morpholino-7,8-dihydro-1,6- naphthyridin-6(5H)- yl)nicotinonitrile | 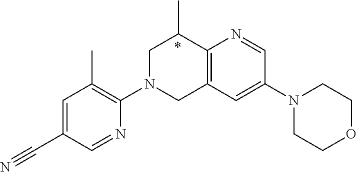 [SMILES: Cc1cc(C#N)cnc1N1Cc2cc(N3CCOCC3)cnc2C(C)C1] | 350.2 |
| 407^{a} | 5-methyl-6-(8-methyl-3- morpholino-7,8-dihydro-1,6- naphthyridin-6(5H)-yl)pyridazine- 3-carbonitrile | 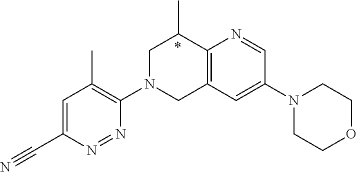 [SMILES: Cc1cc(C#N)nnc1N1Cc2cc(N3CCOCC3)cnc2C(C)C1] | 351.2 |
| 408^{b} | 5-methyl-6-(8-methyl-3- morpholino-7,8-dihydro-1,6- naphthyridin-6(5H)- yl)nicotinonitrile | 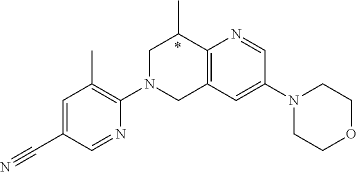 [SMILES: Cc1cc(C#N)cnc1N1Cc2cc(N3CCOCC3)cnc2C(C)C1] | 350.4 |
| 409 | 5-methyl-6-(3-(4-methyl-3- oxopiperazin-1-yl)-7,8-dihydro-1,6- naphthyridin-6(5H)- yl)nicotinonitrile | 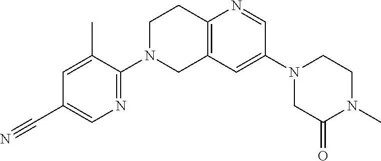 [SMILES: Cc1cc(C#N)cnc1N1CCc2ncc(N3CCN(C)C(=O)C3)cc2C1] | 363 |
| 410 | 5-methyl-6-(3-(4-methyl-3- oxopiperazin-1-yl)-7,8-dihydro-1,6- naphthyridin-6(5H)-yl)pyridazine- 3-carbonitrile | 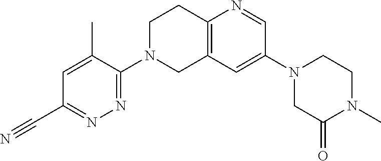 [SMILES: Cc1cc(C#N)nnc1N1CCc2ncc(N3CCN(C)C(=O)C3)cc2C1] | 364 |
| 411 | 6-(3-(6,7-dihydroimidazo[1,2- a]pyrimidin-8(5H)-yl)-7,8-dihydro- 1,6-naphthyridin-6(5H)-yl)-5- methylnicotinonitrile | 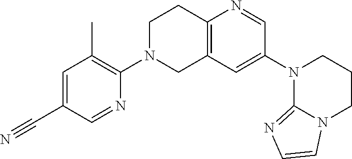 [SMILES: Cc1cc(C#N)cnc1N1CCc2ncc(N3CCCn4ccnc43)cc2C1] | 372 |
| 412 | 5-methyl-6-(3-(2-(trifluoromethyl)- 6,7-dihydropyrazolo[1,5- a]pyrimidin-4(5H)-yl)-7,8-dihydro- 1,6-naphthyridin-6(5H)- yl)nicotinonitrile | 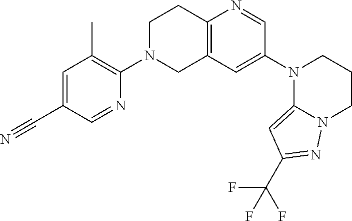 [SMILES: Cc1cc(C#N)cnc1N1CCc2ncc(N3CCCn4nc(C(F)(F)F)cc43)cc2C1] | 440 |
| 413 | 5-methyl-6-(3-(2-(trifluoromethyl)- 6,7-dihydropyrazolo[1,5- a]pyrimidin-4(5H)-yl)-7,8-dihydro- 1,6-naphthyridin-6(5H)- yl)pyridazine-3-carbonitrile | 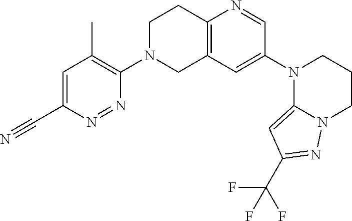 [SMILES: Cc1cc(C#N)nnc1N1CCc2ncc(N3CCCn4nc(C(F)(F)F)cc43)cc2C1] | 441 |
| 414 | 6-(3-(2-(tert-butyl)-6,7- dihydropyrazolo[1,5-a]pyrimidin- 4(5H)-yl)-7,8-dihydro-1,6- naphthyridin-6(5H)-yl)-5- methylnicotinonitrile | 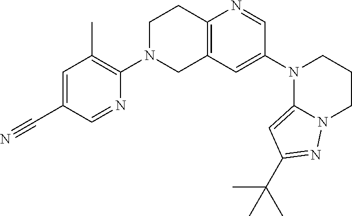 [SMILES: Cc1cc(C#N)cnc1N1CCc2ncc(N3CCCn4nc(C(C)(C)C)cc43)cc2C1] | 428.2 |
| 415 | 6-(3-(2-cyclopropyl-6,7- dihydropyrazolo[1,5-a]pyrimidin- 4(5H)-yl)-7,8-dihydro-1,6- naphthyridin-6(5H)-yl)-5- methylnicotinonitrile | 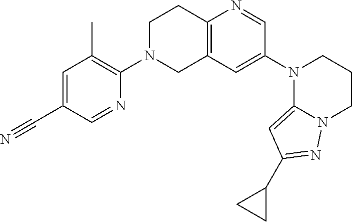 [SMILES: Cc1cc(C#N)cnc1N1CCc2ncc(N3CCCn4nc(C5CC5)cc43)cc2C1] | 412.2 |
| 416 | 6-(3-(6,7-dihydropyrazolo[1,5- a]pyrimidin-4(5H)-yl-5,5,6,6,7,7- d6)-7,8-dihydro-1,6-naphthyridin- 6(5H)-yl)-5-methylpyridazine-3- carbonitrile | 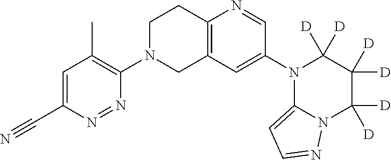 [SMILES: [2H]C1([2H])N(c2cnc3c(c2)CN(c2nnc(C#N)cc2C)CC3)c2ccnn2C([2H])([2H])C1([2H])[2H]] | 379.4 |
| 417 | 6-(3-(6,7-dihydropyrazolo[1,5- a]pyrimidin-4(5H)-yl-5,5,6,6,7,7- d₆)-7,8-dihydro-1,6-naphthyridin- 6(5H)-yl)-5-methylnicotinonitrile | 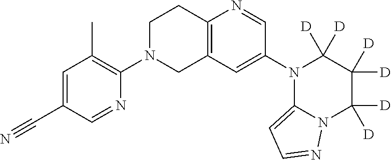 [SMILES: [2H]C1([2H])N(c2cnc3c(c2)CN(c2ncc(C#N)cc2C)CC3)c2ccnn2C([2H])([2H])C1([2H])[2H]] | 378.2 |
| 418 | 6-(3-(2-(tert-butyl)-6,7- dihydropyrazolo[1,5-a]pyrimidin- 4(5H)-yl)-7,8-dihydro-1,6- naphthyridin-6(5H)-yl)-5- methylpyridazine-3-carbonitrile | 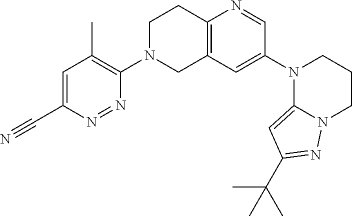 [SMILES: Cc1cc(C#N)nnc1N1CCc2ncc(N3CCCn4nc(C(C)(C)C)cc43)cc2C1] | 429.2 |
| 419 | 6-(3-(2-cyclopropyl-6,7- dihydropyrazolo[1,5-a]pyrimidin- 4(5H)-yl)-7,8-dihydro-1,6- naphthyridin-6(5H)-yl)-5- methylpyridazine-3-carbonitrile | 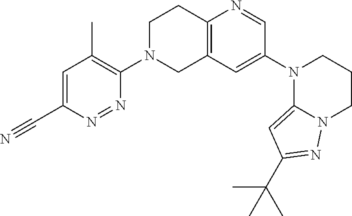 [SMILES: Cc1cc(C#N)nnc1N1CCc2ncc(N3CCCn4nc(C(C)(C)C)cc43)cc2C1] | 413.2 |
| 420 | 5-methyl-6-(3-(4-methyl-2- oxopiperazin-1-yl)-7,8-dihydro-1,6- naphthyridin-6(5H)- yl)nicotinonitrile | 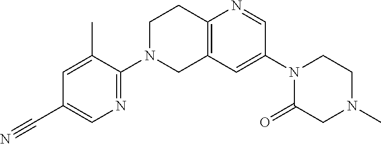 [SMILES: Cc1cc(C#N)cnc1N1CCc2ncc(N3CCN(C)CC3=O)cc2C1] | 363 |
| 421 | 5-methyl-6-(3-(methyl-d₃)-7,8- dihydro-1,6-naphthyridin-6(5H)- yl)pyridazine-3-carbonitrile | 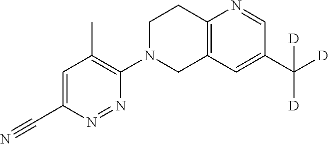 [SMILES: [2H]C([2H])([2H])c1cnc2c(c1)CN(c1nnc(C#N)cc1C)CC2] | 269.2 |
| 422 | 5-methyl-6-(3-(2,2,2- trifluoroethyl)-7,8-dihydro-1,6- naphthyridin-6(5H)-yl)pyridazine- 3-carbonitrile | 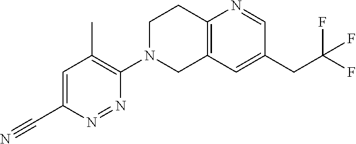 [SMILES: Cc1cc(C#N)nnc1N1CCc2ncc(CC(F)(F)F)cc2C1] | 334.3 |
| 423 | 5-methyl-6-(3-(2,2,2- trifluoroethyl)-7,8-dihydro-1,6- naphthyridin-6(5H)- yl)nicotinonitrile | 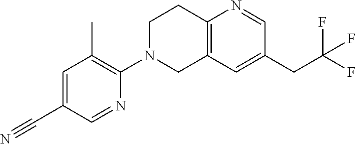 [SMILES: Cc1cc(C#N)cnc1N1CCc2ncc(CC(F)(F)F)cc2C1] | 333.3 |
| 424 | 5-methyl-6-(3-(1,1, 1-trifluoro-2- hydroxypropan-2-yl)-7,8-dihydro- 1,6-naphthyridin-6(5H)- yl)pyridazine-3-carbonitrile | 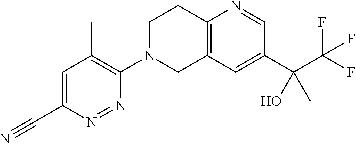 [SMILES: Cc1cc(C#N)nnc1N1CCc2ncc(C(C)(O)C(F)(F)F)cc2C1] | 364.4 |
| 425 | 4,5-dimethyl-6-(3-(1,1,1-trifluoro- 2-hydroxypropan-2-yl)-7,8- dihydro-1,6-naphthyridin-6(5H)- yl)pyridazine-3-carbonitrile | 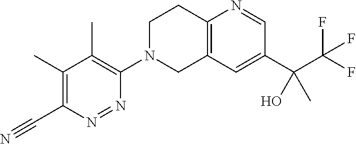 [SMILES: Cc1c(C#N)nnc(N2CCc3ncc(C(C)(O)C(F)(F)F)cc3C2)c1C] | 378.4 |
| 426 | 5-methyl-6-(3-(1,1,1-trifluoro-2- hydroxypropan-2-yl)-7,8-dihydro- 1,6-naphthyridin-6(5H)- yl)nicotinonitrile | 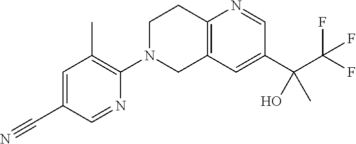 [SMILES: Cc1cc(C#N)cnc1N1CCc2ncc(C(C)(O)C(F)(F)F)cc2C1] | 363.4 |
| 427 | 5-methyl-6-(3-(2-phenoxyethoxy)- 7,8-dihydro-1,6-naphthyridin- 6(5H)-yl)nicotinonitrile | 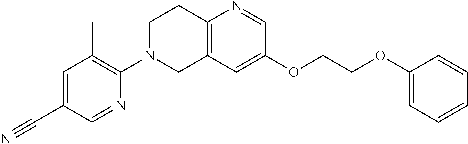 [SMILES: Cc1cc(C#N)cnc1N1CCc2ncc(OCCOc3ccccc3)cc2C1] | 403.3 |
| 428 | 6-(3-(2-(4-fluorophenoxy)ethoxy)- 7,8-dihydro-1,6-naphthyridin- 6(5H)-yl)-5-methylnicotinonitrile | 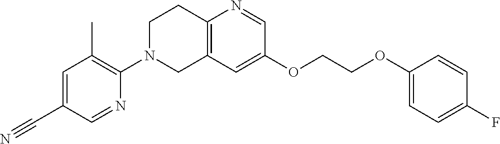 [SMILES: Cc1cc(C#N)cnc1N1CCc2ncc(OCCOc3ccc(F)cc3)cc2C1] | 405.4 |
| 429 | 6-(3-(2-(2-fluorophenoxy)ethoxy)- 7,8-dihydro-1,6-naphthyridin- 6(5H)-yl)-5-methylnicotinonitrile | 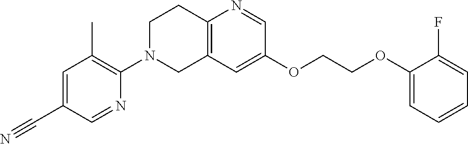 [SMILES: Cc1cc(C#N)cnc1N1CCc2ncc(OCCOc3ccccc3F)cc2C1] | 405.4 |
| 430 | 5-methyl-6-(3-(2-(o- tolyloxy)ethoxy)-7,8-dihydro-1,6- naphthyridin-6(5H)- yl)nicotinonitrile | 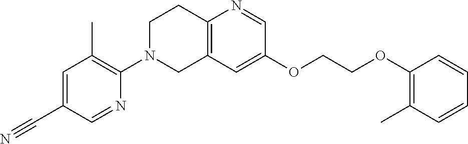 [SMILES: Cc1ccccc1OCCOc1cnc2c(c1)CN(c1ncc(C#N)cc1C)CC2] | 401.4 |
| 431 | 5-methyl-6-(3-(2-(p- tolyloxy)ethoxy)-7,8-dihydro-1,6- naphthyridin-6(5H)- yl)nicotinonitrile | 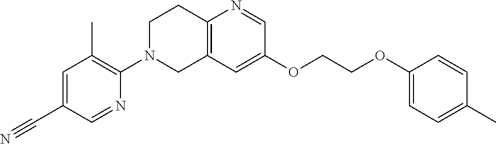 [SMILES: Cc1ccc(OCCOc2cnc3c(c2)CN(c2ncc(C#N)cc2C)CC3)cc1] | 401.4 |
| 432 | 5-methyl-6-(3-(3- phenoxypropoxy)-7,8-dihydro-1,6- naphthyridin-6(5H)- yl)nicotinonitrile | 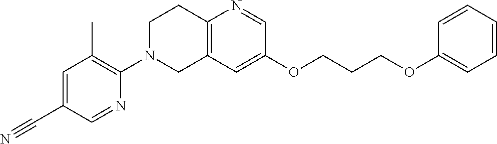 [SMILES: Cc1cc(C#N)cnc1N1CCc2ncc(OCCCOc3ccccc3)cc2C1] | 401.4 |
| 433 | 6-(3-(2-(cyclopentyloxy)ethoxy)- 7,8-dihydro-1,6-naphthyridin- 6(5H)-yl)-5-methylnicotinonitrile | 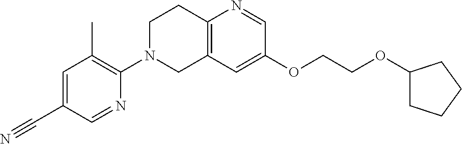 [SMILES: Cc1cc(C#N)cnc1N1CCc2ncc(OCCOC3CCCC3)cc2C1] | 379.4 |
| 434 | 6-(3-(2-methoxyethoxy)-7,8- dihydro-1,6-naphthyridin-6(5H)- yl)-5-methylnicotinonitrile | 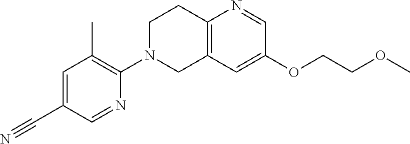 [SMILES: COCCOc1cnc2c(c1)CN(c1ncc(C#N)cc1C)CC2] | 325.5 |
| 435 | 4,5-dimethyl-6-(3-(5- methylpyridin-3-yl)-7,8-dihydro- 1,6-naphthyridin-6(5H)- yl)pyridazine-3-carbonitrile | 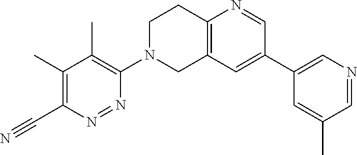 [SMILES: Cc1cncc(-c2cnc3c(c2)CN(c2nnc(C#N)c(C)c2C)CC3)c1] | 357.4 |
| 436 | 6-(3-(6-fluoro-4-methylpyridin-3- yl)-7,8-dihydro-1,6-naphthyridin- 6(5H)-yl)-5-methylnicotinonitrile | 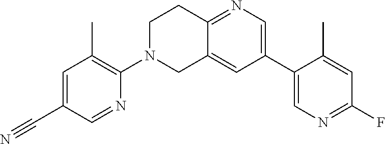 [SMILES: Cc1cc(F)ncc1-c1cnc2c(c1)CN(c1ncc(C#N)cc1C)CC2] | 360 |
| 437 | 2-(3-(6-fluoro-4-methylpyridin-3- yl)-7,8-dihydro-1,6-naphthyridin- 6(5H)-yl)-3- methylisonicotinonitrile | 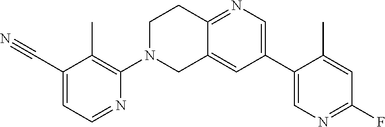 [SMILES: Cc1cc(F)ncc1-c1cnc2c(c1)CN(c1nccc(C#N)c1C)CC2] | 360 |
| 438 | 5-methyl-6-(3-methyl-7,8-dihydro- 1,6-naphthyridin-6(5H)-yl)-N- (pyridin-4-yl)nicotinamide | 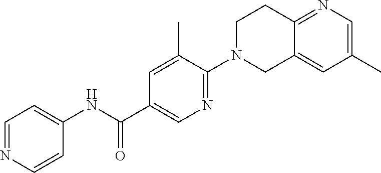 [SMILES: Cc1cnc2c(c1)CN(c1ncc(C(=O)Nc3ccncc3)cc1C)CC2] | 360.2 |
| 439 | 5-methyl-6-(3-methyl-7,8-dihydro- 1,6-naphthyridin-6(5H)-yl)-N- (thiazol-5-ylmethyl)nicotinamide | 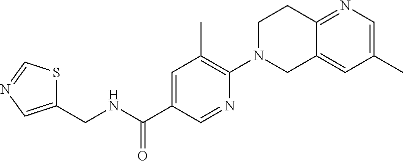 [SMILES: Cc1cnc2c(c1)CN(c1ncc(C(=O)NCc3cncs3)cc1C)CC2] | 380.2 |
| 440 | 5-methyl-6-(3-methyl-7,8-dihydro- 1,6-naphthyridin-6(5H)-yl)-N- (pyridin-4-ylmethyl)nicotinamide | 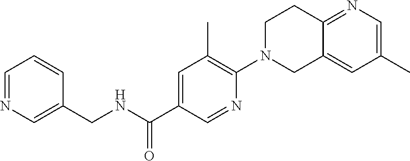 [SMILES: Cc1cnc2c(c1)CN(c1ncc(C(=O)NCc3cccnc3)cc1C)CC2] | 374.2 |
| 441 | 5-methyl-6-(3-methyl-7,8-dihydro- 1,6-naphthyridin-6(5H)-yl)-N-((3- methylpyridin-4- yl)methyl)nicotinamide | 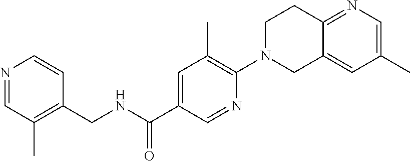 [SMILES: Cc1cnc2c(c1)CN(c1ncc(C(=O)NCc3ccncc3C)cc1C)CC2] | 388.2 |
| 442 | 5-methyl-N-(pyridin-4-yl)-6-(3- (trifluoromethyl)-7,8-dihydro-1,6- naphthyridin-6(5H)- yl)nicotinamide | 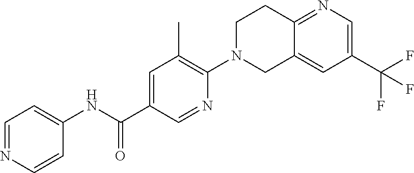 [SMILES: Cc1cc(C(=O)Nc2ccncc2)cnc1N1CCc2ncc(C(F)(F)F)cc2C1] | 414.2 |
| 443 | 5-methyl-N-(thiazol-5-ylmethyl)-6- (3-(trifluoromethyl)-7,8-dihydro- 1,6-naphthyridin-6(5H)- yl)nicotinamide | 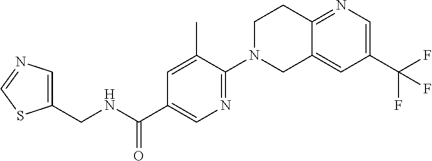 [SMILES: Cc1cc(C(=O)NCc2cncs2)cnc1N1CCc2ncc(C(F)(F)F)cc2C1] | 434 |
| 444 | 5-methyl-N-(pyridin-4-ylmethyl)-6- (3-(trifluoromethyl)-7,8-dihydro- 1,6-naphthyridin-6(5H)- yl)nicotinamide | 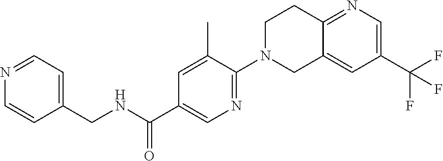 [SMILES: Cc1cc(C(=O)NCc2ccncc2)cnc1N1CCc2ncc(C(F)(F)F)cc2C1] | 428.2 |
| 445 | 5-methyl-N-((3-methylpyridin-4- yl)methyl)-6-(3-(trifluoromethyl)- 7,8-dihydro-1,6-naphthyridin- 6(5H)-yl)nicotinamide | 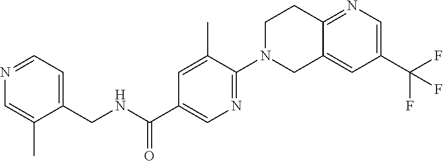 [SMILES: Cc1cnccc1CNC(=O)c1cnc(N2CCc3ncc(C(F)(F)F)cc3C2)c(C)c1] | 442.2 |
| 446 | 5-methyl-N-(quinolin-3-ylmethyl)- 6-(3-(trifluoromethyl)-7,8-dihydro- 1,6-naphthyridin-6(5H)- yl)nicotinamide | 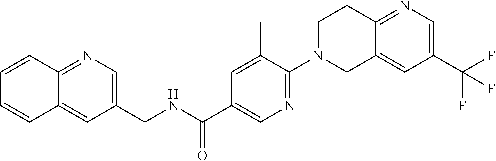 [SMILES: Cc1cc(C(=O)NCc2cnc3ccccc3c2)cnc1N1CCc2ncc(C(F)(F)F)cc2C1] | 478.2 |
| 447 | N-(benzo[b]thiophen-3-ylmethyl)- 5-methyl-6-(3-(trifluoromethyl)- 7,8-dihydro-1,6-naphthyridin- 6(5H)-yl)nicotinamide | 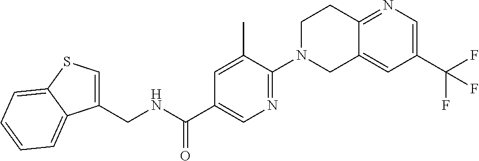 [SMILES: Cc1cc(C(=O)NCc2csc3ccccc23)cnc1N1CCc2ncc(C(F)(F)F)cc2C1] | 483 |
| 448 | 5-methyl-6-(3-(trifluoromethyl)- 7,8-dihydro-1,6-naphthyridin- 6(5H)-yl)nicotinamide | 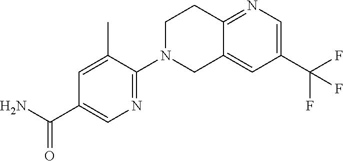 [SMILES: Cc1cc(C(N)=O)cnc1N1CCc2ncc(C(F)(F)F)cc2C1] | 337.2 |
| 449 | 5-methyl-6-(3-((1-methyl-1H- pyrazol-5-yl)amino)-7,8-dihydro- 1,6-naphthyridin-6(5H)-yl)-N- (thiazol-5-ylmethyl)nicotinamide | 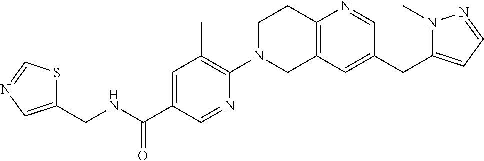 [SMILES: Cc1cc(C(=O)NCc2cncs2)cnc1N1CCc2ncc(Cc3ccnn3C)cc2C1] | 461.3 |
| 450 | 6-(3-(1,3-dimethyl-1H-pyrazol-4- yl)-7,8-dihydro-1,6-naphthyridin- 6(5H)-yl)-5-methyl-N-(pyridin-4- yl)nicotinamide | 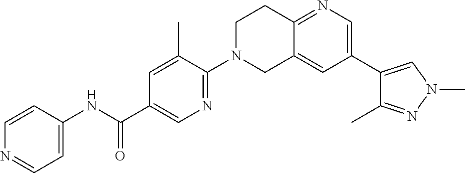 [SMILES: Cc1cc(C(=O)Nc2ccncc2)cnc1N1CCc2ncc(-c3cn(C)nc3C)cc2C1] | 440.2 |
| 451 | 6-(3-(1,3-dimethyl-1/-pyrazol-4- yl)-7,8-dihydro-1,6-naphthyridin- 6(5H)-yl)-5-methyl-N-((3- methylpyridin-4- yl)methyl)nicotinamide | 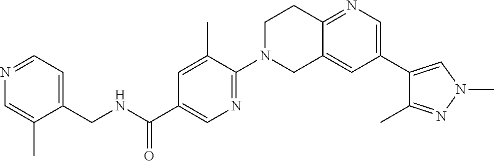 [SMILES: Cc1cnccc1CNC(=O)c1cnc(N2CCc3ncc(-c4cn(C)nc4C)cc3C2)c(C)c1] | 468.2 |
| 452 | N-(benzo[b]thiophen-3-ylmethyl)- 6-(3-(1,3-dimethyl-1H-pyrazol-4- yl)-7,8-dihydro-1,6-naphthyridin- 6(5H)-yl)-5-methylnicotinamide | 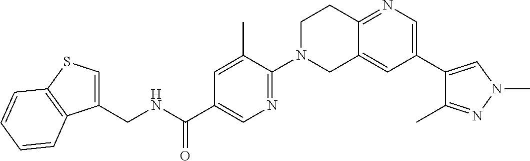 [SMILES: Cc1cc(C(=O)NCc2csc3ccccc23)cnc1N1CCc2ncc(-c3cn(C)nc3C)cc2C1] | 509.2 |
| 453 | N-(benzo[d]thiazol-2-ylmethyl)-6- (3-(1,3-dimethyl-1H-pyrazol-4-yl)- 7,8-dihydro-1,6-naphthyridin- 6(5H)-yl)-5-methylnicotinamide | 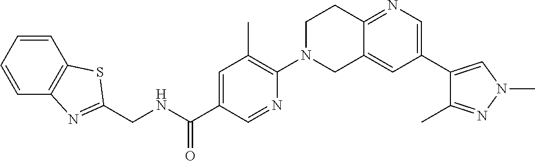 [SMILES: Cc1cc(C(=O)NCc2nc3ccccc3s2)cnc1N1CCc2ncc(-c3cn(C)nc3C)cc2C1] | 510.2 |
| 454 | 6-(3-(1,3-dimethyl-1/-pyrazol-4- yl)-7,8-dihydro-1,6-naphthyridin- 6(5H)-yl)-N,5-dimethyl-N-(pyridin- 4-ylmethyl)nicotinamide | 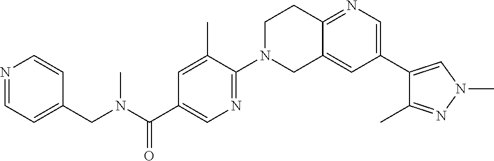 [SMILES: Cc1cc(C(=O)N(C)Cc2ccncc2)cnc1N1CCc2ncc(-c3cn(C)nc3C)cc2C1] | 468.5 |
| 455 | N-((2-chlorothiazol-5-yl)methyl)-6- (3-(1,3-dimethyl-1H-pyrazol-4-yl)- 7,8-dihydro-1,6-naphthyridin- 6(5H)-yl)-5-methylnicotinamide | 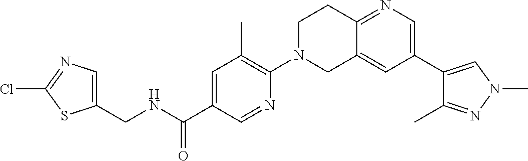 [SMILES: Cc1cc(C(=O)NCc2cnc(Cl)s2)cnc1N1CCc2ncc(-c3cn(C)nc3C)cc2C1] | 494.2 |
| 456 | N-((6-cyanopyridin-3-yl)methyl)-6- (3-(1,3-dimethyl-1H-pyrazol-4-yl)- 7,8-dihydro-1,6-naphthyridin- 6(5H)-yl)-5-methylnicotinamide | 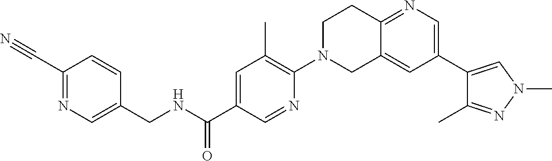 [SMILES: Cc1cc(C(=O)NCc2ccc(C#N)nc2)cnc1N1CCc2ncc(-c3cn(C)nc3C)cc2C1] | 479.4 |
| 457 | 6-(3-(1,3-dimethyl-1H-pyrazol-4- yl)-7,8-dihydro-1,6-naphthyridin- 6(5H)-yl)-5-methyl-N-(pyridin-3- ylmethyl)nicotinamide | 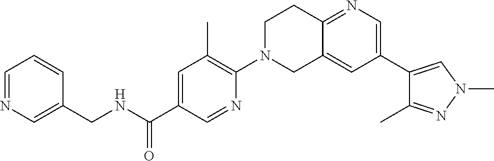 [SMILES: Cc1cc(C(=O)NCc2cccnc2)cnc1N1CCc2ncc(-c3cn(C)nc3C)cc2C1] | 454.4 |
| 458 | 6-(3-(1,3-dimethyl-1H-pyrazol-4- yl)-7,8-dihydro-1,6-naphthyridin- 6(5H)-yl)-5-methyl-N-(pyridin-2- ylmethyl)nicotinamide | 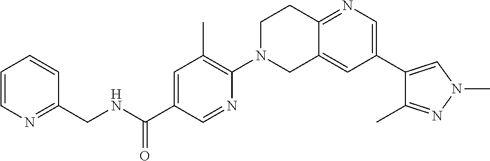 [SMILES: Cc1cc(C(=O)NCc2ccccn2)cnc1N1CCc2ncc(-c3cn(C)nc3C)cc2C1] | 454.4 |
| 459 | 6-(3-(1,3-dimethyl-1H-pyrazol-4- yl)-7,8-dihydro-1,6-naphthyridin- 6(5H)-yl)-5-methyl-N-((tetrahydro- 2H-pyran-4-yl)methyl)nicotinamide | 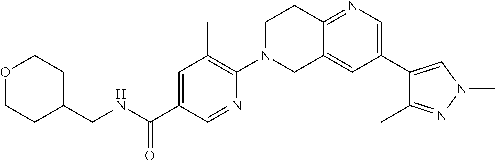 [SMILES: Cc1cc(C(=O)NCC2CCOCC2)cnc1N1CCc2ncc(-c3cn(C)nc3C)cc2C1] | 461.5 |
| 460 | 6-(3-(1,3-dimethyl-1H-pyrazol-4- yl)-7,8-dihydro-1,6-naphthyridin- 6(5H)-yl)-N-isobutyl-5- methylnicotinamide | 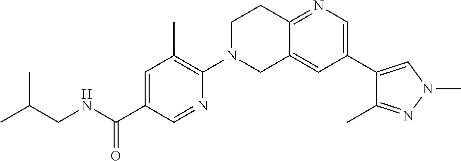 [SMILES: Cc1cc(C(=O)NCC(C)C)cnc1N1CCc2ncc(-c3cn(C)nc3C)cc2C1] | 419.4 |
| 461 | 6-(3-(1,3-dimethyl-1H-pyrazol-4- yl)-7,8-dihydro-1,6-naphthyridin- 6(5H)-yl)-N-(2-hydroxy-2- methylpropyl)-5- methylnicotinamide | 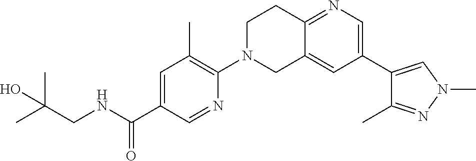 [SMILES: Cc1cc(C(=O)NCC(C)(C)O)cnc1N1CCc2ncc(-c3cn(C)nc3C)cc2C1] | 435.4 |
| 462 | N-((2,6-dichloropyridin-3- yl)methyl)-6-(3-(1,3-dimethyl-1H- pyrazol-4-yl)-7,8-dihydro-1,6- naphthyridin-6(5H)-yl)-5- methylnicotinamide | 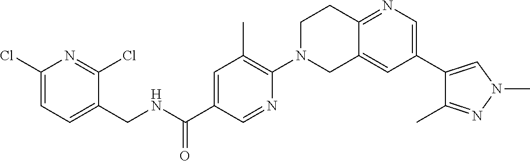 [SMILES: Cc1cc(C(=O)NCc2ccc(Cl)nc2Cl)cnc1N1CCc2ncc(-c3cn(C)nc3C)cc2C1] | 522.3 |
| 463 | N-(benzo[b]thiophen-3-ylmethyl)- 4,5-dimethyl-6-(3- (trifluoromethyl)-7,8-dihydro-1,6- naphthyridin-6(5H)-yl)pyridazine- 3-carboxamide | 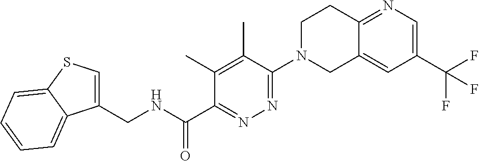 [SMILES: Cc1c(C(=O)NCc2csc3ccccc23)nnc(N2CCc3ncc(C(F)(F)F)cc3C2)c1C] | 498.3 |
| 464 | 4,5-dimethyl-N-((3-methylpyridin- 4-yl)methyl)-6-(3- (trifluoromethyl)-7,8-dihydro-1,6- naphthyridin-6(5H)-yl)pyridazine- 3-carboxamide | 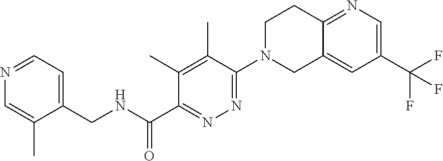 [SMILES: Cc1cnccc1CNC(=O)c1nnc(N2CCc3ncc(C(F)(F)F)cc3C2)c(C)c1C] | 457.4 |
| 465 | 4,5-dimethyl-N-(thiazol-5- ylmethyl)-6-(3-(trifluoromethyl)- 7,8-dihydro-1,6-naphthyridin- 6(5H)-yl)pyridazine-3-carboxamide | 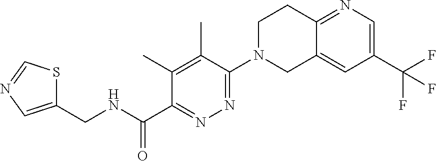 [SMILES: Cc1c(C(=O)NCc2cncs2)nnc(N2CCc3ncc(C(F)(F)F)cc3C2)c1C] | 449.2 |
| 466 | 4,5-dimethyl-N-(pyridin-4-yl)-6-(3- (trifluoromethyl)-7,8-dihydro-1,6- naphthyridin-6(5H)-yl)pyridazine- 3-carboxamide | 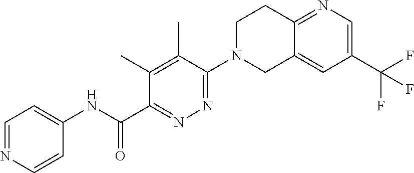 [SMILES: Cc1c(C(=O)Nc2ccncc2)nnc(N2CCc3ncc(C(F)(F)F)cc3C2)c1C] | 429.2 |
| 467 | 4,5-dimethyl-N-(pyridin-4- ylmethyl)-6-(3-(trifluoromethyl)- 7,8-dihydro-1,6-naphthyridin- 6(5H)-yl)pyridazine-3-carboxamide | 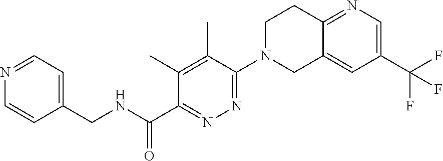 [SMILES: Cc1c(C(=O)NCc2ccncc2)nnc(N2CCc3ncc(C(F)(F)F)cc3C2)c1C] | 443.3 |
| 468 | 4,5-dimethyl-6-(3-methyl-7,8- dihydro-1,6-naphthyridin-6(5H)- yl)-N-(thiazol-5- ylmethyl)pyridazine-3-carboxamide | 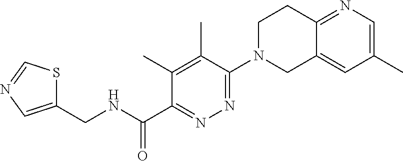 [SMILES: Cc1cnc2c(c1)CN(c1nnc(C(=O)NCc3cncs3)c(C)c1C)CC2] | 395.3 |
| 469 | 4,5-dimethyl-6-(3-methyl-7,8- dihydro-1,6-naphthyridin-6(5H)- yl)-N-(pyridin-4-yl)pyridazine-3- carboxamide | 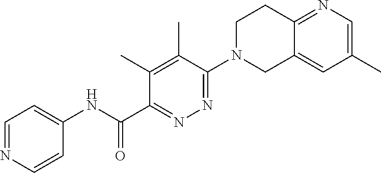 [SMILES: Cc1cnc2c(c1)CN(c1nnc(C(=O)Nc3ccncc3)c(C)c1C)CC2] | 375.4 |
| 470 | N-(benzo[b]thiophen-3-ylmethyl)- 4,5-dimethyl-6-(3-methyl-7,8- dihydro-1,6-naphthyridin-6(5H)- yl)pyridazine-3-carboxamide | 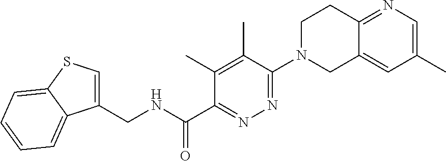 [SMILES: Cc1cnc2c(c1)CN(c1nnc(C(=O)NCc3csc4ccccc34)c(C)c1C)CC2] | 444.3 |
| 471 | 4,5-dimethyl-6-(3-methyl-7,8- dihydro-1,6-naphthyridin-6(5H)- yl)-N-(quinolin-3- ylmethyl)pyridazine-3-carboxamide | 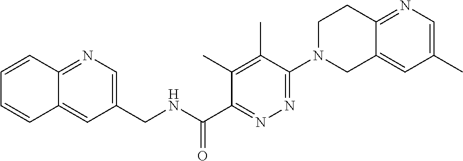 [SMILES: Cc1cnc2c(c1)CN(c1nnc(C(=O)NCc3cnc4ccccc4c3)c(C)c1C)CC2] | 439.4 |
| 472 | 4,5-dimethyl-6-(3-methyl-7,8- dihydro-1,6-naphthyridin-6(5H)- yl)-N-((3-methylpyridin-4- yl)methyl)pyridazine-3- carboxamide | 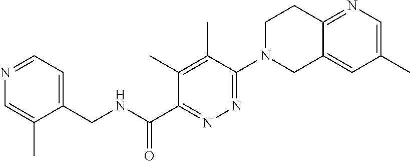 [SMILES: Cc1cnc2c(c1)CN(c1nnc(C(=O)NCc3ccncc3C)c(C)c1C)CC2] | 403.3 |
| 473 | 5-methyl-6-(3-((1-methyl-1H- pyrazol-5-yl)amino)-7,8-dihydro- 1,6-naphthyridin-6(5H)-yl)-N- (pyridin-4-ylmethyl)nicotinamide | 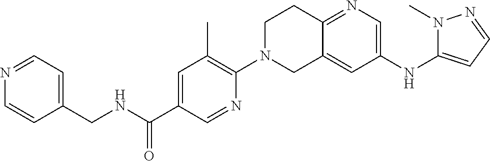 [SMILES: Cc1cc(C(=O)NCc2ccncc2)cnc1N1CCc2ncc(Nc3ccnn3C)cc2C1] | 455.4 |
| 474 | 5-methyl-6-(3-((1-methyl-1H- pyrazol-5-yl)amino)-7,8-dihydro- 1,6-naphthyridin-6(5H)-yl)-N-((3- methylpyridin-4- yl)methyl)nicotinamide | 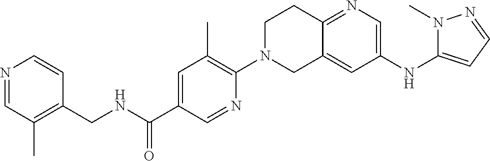 [SMILES: Cc1cnccc1CNC(=O)c1cnc(N2CCc3ncc(Nc4ccnn4C)cc3C2)c(C)c1] | 469.5 |
| 475 | 6-(3-(1,3-dimethyl-1H-pyrazol-4- yl)-7,8-dihydro-1,6-naphthyridin- 6(5H)-yl)-5-methyl-N-(2- oxaspiro[3.3 ]heptan-6- yl)nicotinamide | 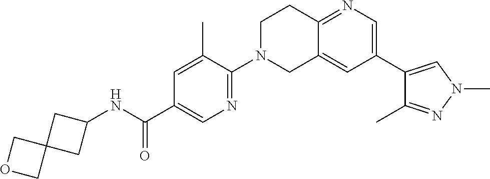 [SMILES: Cc1cc(C(=O)NC2CC3(COC3)C2)cnc1N1CCc2ncc(-c3cn(C)nc3C)cc2C1] | 459.2 |
| 476 | N-(3,3-difluorocyclobutyl)-6-(3- (1,3-dimethyl-1H-pyrazol-4-yl)- 7,8-dihydro-1,6-naphthyridin- 6(5H)-yl)-5-methylnicotinamide | 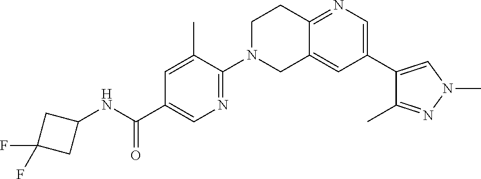 [SMILES: Cc1cc(C(=O)NC2CC(F)(F)C2)cnc1N1CCc2ncc(-c3cn(C)nc3C)cc2C1] | 453.4 |
| 477 | 6-(3-(1,3-dimethyl-1H-pyrazol-4- yl)-7,8-dihydro-1,6-naphthyridin- 6(5H)-yl)-N-(3- fluorobicyclo[1.1.1 ]pentan-1-yl)-5- methylnicotinamide | 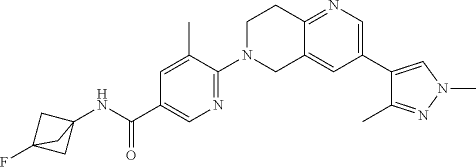 [SMILES: Cc1cc(C(=O)NC23CC(F)(C2)C3)cnc1N1CCc2ncc(-c3cn(C)nc3C)cc2C1] | 447.3 |
| 478 | N-((6-(1H-imidazol-1-yl)pyridin-3- yl)methyl)-6-(3-(1,3-dimethyl-1H- pyrazol-4-yl)-7,8-dihydro-1,6- naphthyridin-6(5/)-yl)-5- methylnicotinamide | 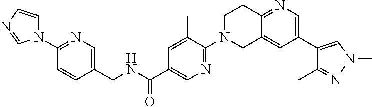 [SMILES: Cc1cc(C(=O)NCc2ccc(-n3ccnc3)nc2)cnc1N1CCc2ncc(-c3cn(C)nc3C)cc2C1] | 520.4 |
| 479 | N-([1,2,4]triazolo[1,5-a]pyridin-6- ylmethyl)-6-(3-(1,3-dimethyl-1/- pyrazol-4-yl)-7,8-dihydro-1,6- naphthyridin-6(5H)-yl)-5- methylnicotinamide | 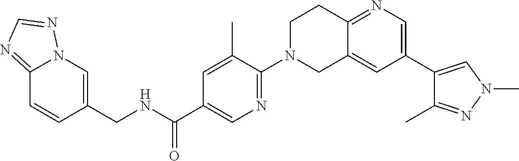 [SMILES: Cc1cc(C(=O)NCc2ccc3ncnn3c2)cnc1N1CCc2ncc(-c3cn(C)nc3C)cc2C1] | 494.4 |
| 480 | 6-(3-(1,3-dimethyl-1H-pyrazol-4- yl)-7,8-dihydro-1,6-naphthyridin- 6(5H)-yl)-5-methyl-N-((6- morpholinopyridin-3- yl)methyl)nicotinamide | 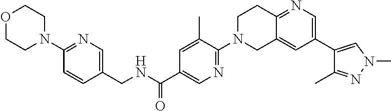 [SMILES: Cc1cc(C(=O)NCc2ccc(N3CCOCC3)nc2)cnc1N1CCc2ncc(-c3cn(C)nc3C)cc2C1] | 539.4 |
| 481 | N-((6-chloropyridin-3-yl)methyl)- 6-(3-(1,3-dimethyl-1H-pyrazol-4- yl)-7,8-dihydro-1,6-naphthyridin- 6(5H)-yl)-5-methylnicotinamide | 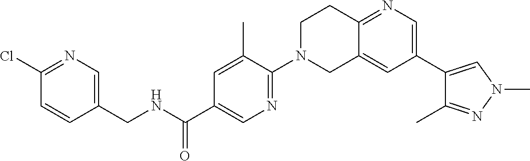 [SMILES: Cc1cc(C(=O)NCc2ccc(Cl)nc2)cnc1N1CCc2ncc(-c3cn(C)nc3C)cc2C1] | 488.4 |
| 482 | 6-(3-(1,3-dimethyl-1H-pyrazol-4- yl)-7,8-dihydro-1,6-naphthyridin- 6(5H)-yl)-5-methyl-N-(pyridin-4- ylmethyl)nicotinamide | 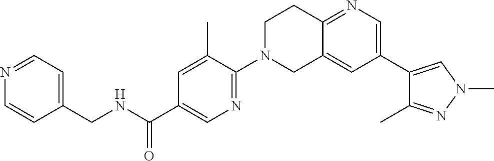 [SMILES: Cc1cc(C(=O)NCc2ccncc2)cnc1N1CCc2ncc(-c3cn(C)nc3C)cc2C1] | 454.4 |
| 483 | 6-(3-(6-cyclopropylpyridin-3-yl)- 7,8-dihydro-1,6-naphthyridin- 6(5H)-yl)-5-methyl-N-(pyridin-4- ylmethyl)nicotinamide | 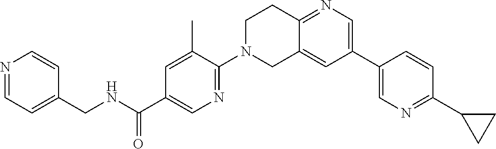 [SMILES: Cc1cc(C(=O)NCc2ccncc2)cnc1N1CCc2ncc(-c3ccc(C4CC4)nc3)cc2C1] | 477.5 |
| 484 | 6-(3-(6-cyclopropylpyridin-3-yl)- 7,8-dihydro-1,6-naphthyridin- 6(5H)-yl)-5-methyl-N-(thiazol-4- ylmethyl)nicotinamide | 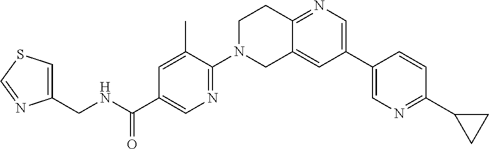 [SMILES: Cc1cc(C(=O)NCc2cscn2)cnc1N1CCc2ncc(-c3ccc(C4CC4)nc3)cc2C1] | 483.3 |
| 485 | 6-(3-(6-(dimethylamino)pyridin-3- yl)-7,8-dihydro-1,6-naphthyridin- 6(5H)-yl)-5-methyl-N-(thiazol-5- ylmethyl)nicotinamide | 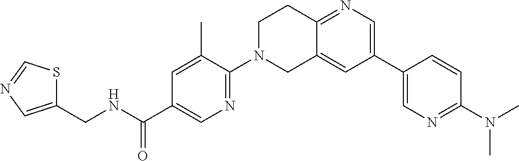 [SMILES: Cc1cc(C(=O)NCc2cncs2)cnc1N1CCc2ncc(-c3ccc(N(C)C)nc3)cc2C1] | 486.4 |
| 486 | 4,5-dimethyl-6-(3-methyl-7,8- dihydro-1,6-naphthyridin-6(5H)- yl)-N-(pyridin-4- ylmethyl)pyridazine-3-carboxamide | 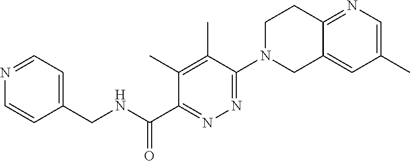 [SMILES: Cc1cnc2c(c1)CN(c1nnc(C(=O)NCc3ccncc3)c(C)c1C)CC2] | 304.4 |
| 487 | 6-(3-(1,3-dimethyl-1H-pyrazol-4- yl)-7,8-dihydro-1,6-naphthyridin- 6(5H)-yl)-5- (trifluoromethyl)nicotinamide | 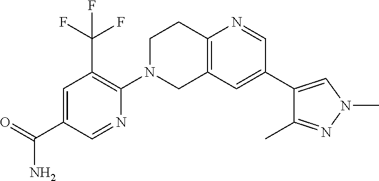 [SMILES: Cc1nn(C)cc1-c1cnc2c(c1)CN(c1ncc(C(N)=O)cc1C(F)(F)F)CC2] | 417 |
| 488 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(pyrrolidin-1- yl)methanone | 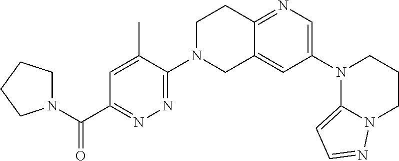 [SMILES: Cc1cc(C(=O)N2CCCC2)nnc1N1CCc2ncc(N3CCCn4nccc43)cc2C1] | 445.4 |
| 489 | 6-(3-(6,7-dihydropyrazolo[1,5- a]pyrimidin-4(5H)-yl)-7,8-dihydro- 1,6-naphthyridin-6(5H)-yl)-5- methyl-N-(thiazol-5- ylmethyl)pyridazine-3-carboxamide | 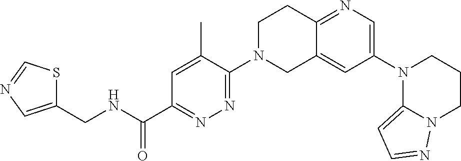 [SMILES: Cc1cc(C(=O)NCc2cncs2)nnc1N1CCc2ncc(N3CCCn4nccc43)cc2C1] | 488.4 |
| 490 | 6-(3-(6,7-dihydropyrazolo[1,5- a]pyrimidin-4(5H)-yl)-7,8-dihydro- 1,6-naphthyridin-6(5H)-yl)-5- methyl-N-(pyridin-4- ylmethyl)pyridazine-3-carboxamide | 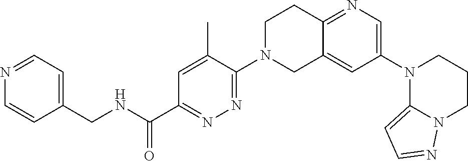 [SMILES: Cc1cc(C(=O)NCc2ccncc2)nnc1N1CCc2ncc(N3CCCn4nccc43)cc2C1] | 482.4 |
| 491 | 6-(3-(6,7-dihydropyrazolo[1,5- a]pyrimidin-4(5H)-yl)-7,8-dihydro- 1,6-naphthyridin-6(5H)-yl)-5- methyl-N-((3-methylpyridin-4- yl)methyl)pyridazine-3- carboxamide | 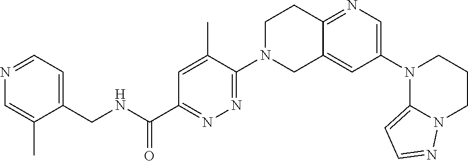 [SMILES: Cc1cnccc1CNC(=O)c1cc(C)c(N2CCc3ncc(N4CCCn5nccc54)cc3C2)nn1] | 496.4 |
| 492 | 5-methyl-N-(thiazol-5-ylmethyl)-6- (3-(trifluoromethyl)-7,8-dihydro- 1,6-naphthyridin-6(5H)- yl)pyridazine-3-carboxamide | 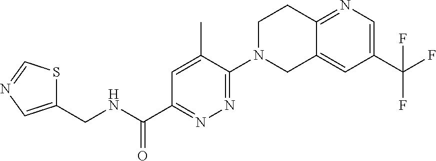 [SMILES: Cc1cc(C(=O)NCc2cncs2)nnc1N1CCc2ncc(C(F)(F)F)cc2C1] | 435.3 |
| 493 | 5-methyl-N-(pyridin-4-ylmethyl)-6- (3-(trifluoromethyl)-7,8-dihydro- 1,6-naphthyridin-6(5H)- yl)pyridazine-3-carboxamide | 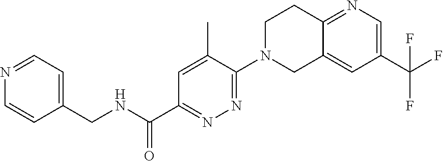 [SMILES: Cc1cc(C(=O)NCc2ccncc2)nnc1N1CCc2ncc(C(F)(F)F)cc2C1] | 429.4 |
| 494 | 5-methyl-N-((3-methylpyridin-4- yl)methyl)-6-(3-(trifluoromethyl)- 7,8-dihydro-1,6-naphthyridin- 6(5H)-yl)pyridazine-3-carboxamide | 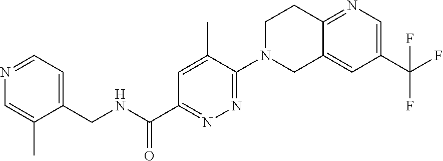 [SMILES: Cc1cnccc1CNC(=O)c1cc(C)c(N2CCc3ncc(C(F)(F)F)cc3C2)nn1] | 443.3 |
| 495 | N-((3-methoxypyridin-4- yl)methyl)-5-methyl-6-(3- (trifluoromethyl)-7,8-dihydro-1,6- naphthyridin-6(5H)-yl)pyridazine- 3-carboxamide | 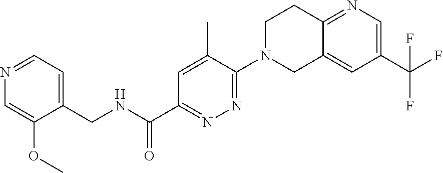 [SMILES: COc1cnccc1CNC(=O)c1cc(C)c(N2CCc3ncc(C(F)(F)F)cc3C2)nn1] | 459.2 |
| 496 | N-((3-fluoropyridin-4-yl)methyl)-5- methyl-6-(3-(trifluoromethyl)-7,8- dihydro-1,6-naphthyridin-6(5H)- yl)pyridazine-3-carboxamide | 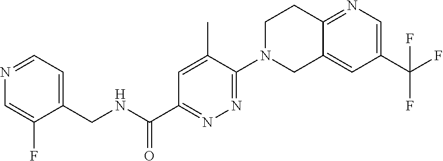 [SMILES: Cc1cc(C(=O)NCc2ccncc2F)nnc1N1CCc2ncc(C(F)(F)F)cc2C1] | 447.3 |
| 497 | N-((3-chloropyridin-4-yl)methyl)- 5-methyl-6-(3-(trifluoromethyl)- 7,8-dihydro-1,6-naphthyridin- 6(5H)-yl)pyridazine-3-carboxamide | 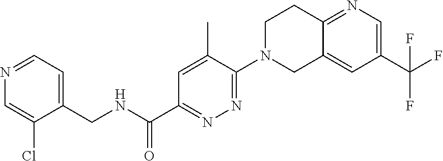 [SMILES: Cc1cc(C(=O)NCc2ccncc2Cl)nnc1N1CCc2ncc(C(F)(F)F)cc2C1] | 463.2 |
| 498 | (3,4-dihydro-2,6-naphthyridin- 2(1H)-yl)(5-methyl-6-(3- (trifluoromethyl)-7,8-dihydro-1,6- naphthyridin-6(5H)-yl)pyridazin-3- yl)methanone | 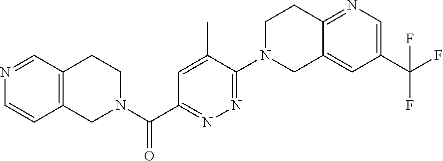 [SMILES: Cc1cc(C(=O)N2CCc3cnccc3C2)nnc1N1CCc2ncc(C(F)(F)F)cc2C1] | 455.3 |
| 499 | (3,4-dihydro-2,7-naphthyridin- 2(1H)-yl)(5-methyl-6-(3- (trifluoromethyl)-7,8-dihydro-1,6- naphthyridin-6(5H)-yl)pyridazin-3- yl)methanone | 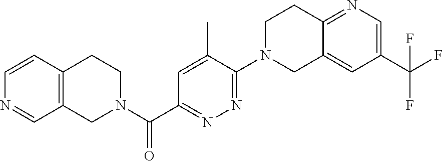 [SMILES: Cc1cc(C(=O)N2CCc3ccncc3C2)nnc1N1CCc2ncc(C(F)(F)F)cc2C1] | 455.3 |
| 500 | (5,8-dihydro-1,7-naphthyridin- 7(6H)-yl)(5-methyl-6-(3- (trifluoromethyl)-7,8-dihydro-1,6- naphthyridin-6(5H)-yl)pyridazin-3- yl)methanone | 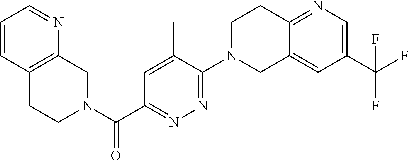 [SMILES: Cc1cc(C(=O)N2CCc3cccnc3C2)nnc1N1CCc2ncc(C(F)(F)F)cc2C1] | 455.3 |
| 501 | 5-methyl-N-(5,6,7,8- tetrahydroquinolin-6-yl)-6-(3- (trifluoromethyl)-7,8-dihydro-1,6- naphthyridin-6(5H)-yl)pyridazine- 3-carboxamide | 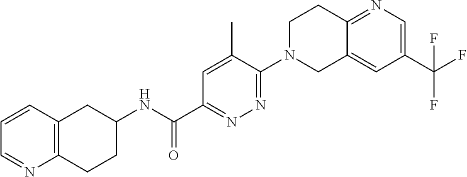 [SMILES: Cc1cc(C(=O)NC2CCc3ncccc3C2)nnc1N1CCc2ncc(C(F)(F)F)cc2C1] | 469.4 |
| 502 | (5-methyl-6-(3-(trifluoromethyl)- 7,8-dihydro-1,6-naphthyridin- 6(5H)-yl)pyridazin-3- yl)(pyrrolidin-1-yl)methanone | 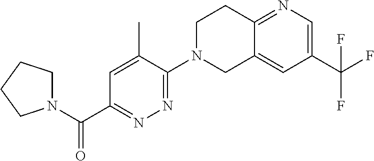 [SMILES: Cc1cc(C(=O)N2CCCC2)nnc1N1CCc2ncc(C(F)(F)F)cc2C1] | 392.3 |
| 503 | (6,7-dihydropyrazolo[1,5- a]pyrimidin-4(5H)-yl)(5-methyl-6- (3-(trifluoromethyl)-7,8-dihydro- 1,6-naphthyridin-6(5H)- yl)pyridazin-3-yl)methanone | 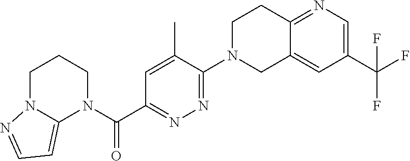 [SMILES: Cc1cc(C(=O)N2CCCn3nccc32)nnc1N1CCc2ncc(C(F)(F)F)cc2C1] | 444.4 |
| 504 | (3-azabicyclo[3.1.0]hexan-3-yl)(5- methyl-6-(3-(trifluoromethyl)-7,8- dihydro-1,6-naphthyridin-6(5H)- yl)pyridazin-3-yl)methanone | 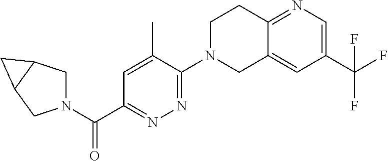 [SMILES: Cc1cc(C(=O)N2CC3CC3C2)nnc1N1CCc2ncc(C(F)(F)F)cc2C1] | 404.3 |
| 505 | 5-methyl-N-(pyridin-4-ylmethyl- d₂)-6-(3-(trifluoromethyl)-7,8- dihydro-1,6-naphthyridin-6(5H)- yl)pyridazine-3-carboxamide | 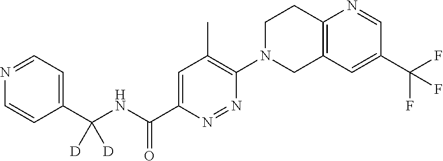 [SMILES: [2H]C([2H])(NC(=O)c1cc(C)c(N2CCc3ncc(C(F)(F)F)cc3C2)nn1)c1ccncc1] | 431.3 |
| 506 | N,5-dimethyl-N-(pyridin-4- ylmethyl)-6-(3-(trifluoromethyl)- 7,8-dihydro-1,6-naphthyridin- 6(5H)-yl)pyridazine-3-carboxamide | 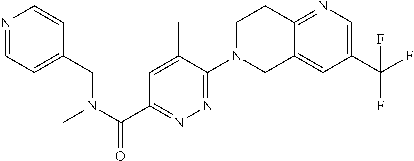 [SMILES: Cc1cc(C(=O)N(C)Cc2ccncc2)nnc1N1CCc2ncc(C(F)(F)F)cc2C1] | 443.3 |
| 507 | 5-methyl-N-(thiazol-4-ylmethyl- d₂)-6-(3-(trifluoromethyl)-7,8- dihydro-1,6-naphthyridin-6(5H)- yl)pyridazine-3-carboxamide | 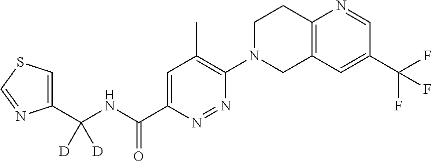 [SMILES: [2H]C([2H])(NC(=O)c1cc(C)c(N2CCc3ncc(C(F)(F)F)cc3C2)nn1)c1cscn1] | 437.2 |
| 508 | 6-(3-(6,7-dihydropyrazolo[1,5- a]pyrimidin-4(5H)-yl)-7,8-dihydro- 1,6-naphthyridin-6(5H)-yl)-5- methyl-N-(thiazol-4-ylmethyl- d2)pyridazine-3-carboxamide | 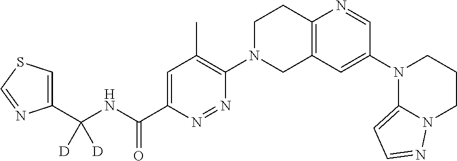 [SMILES: [2H]C([2H])(NC(=O)c1cc(C)c(N2CCc3ncc(N4CCCn5nccc54)cc3C2)nn1)c1cscn1] | 490.4 |
| 509 | 6-(3-(6,7-dihydropyrazolo[1,5- a]pyrimidin-4(5H)-yl)-7,8-dihydro- 1,6-naphthyridin-6(5H)-yl)-5- methyl-N-(pyridin-4-ylmethyl- d₂)pyridazine-3-carboxamide | 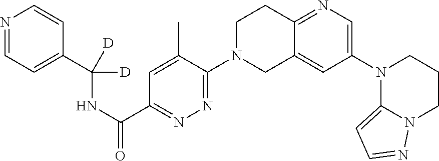 [SMILES: [2H]C([2H])(NC(=O)c1cc(C)c(N2CCc3ncc(N4CCCn5nccc54)cc3C2)nn1)c1ccncc1] | 391.2 |
| 510 | N-(thiazol-5-ylmethyl)-6-(3- (trifluoromethyl)-7,8-dihydro-1,6- naphthyridin-6(5H)-yl)pyridazine- 3-carboxamide | 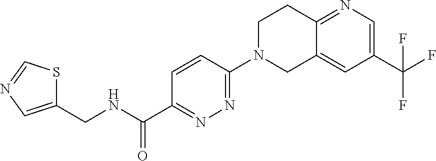 [SMILES: O=C(NCc1cncs1)c1ccc(N2CCc3ncc(C(F)(F)F)cc3C2)nn1] | 421.4 |
| 511 | N-(pyridin-4-ylmethyl)-6-(3- (trifluoromethyl)-7,8-dihydro-1,6- naphthyridin-6(5H)-yl)pyridazine- 3-carboxamide | 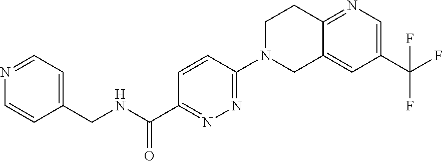 [SMILES: O=C(NCc1ccncc1)c1ccc(N2CCc3ncc(C(F)(F)F)cc3C2)nn1] | 415.3 |
| 512 | (1,3-dihydro-2H-pyrrolo[3,4- c]pyridin-2-yl)(6-(3-(6,7- dihydropyrazolo[1,5-a]pyrimidin- 4(5H)-yl)-7,8-dihydro-1,6- naphthyridin-6(5H)-yl)-5- methylpyridazin-3-yl)methanone | 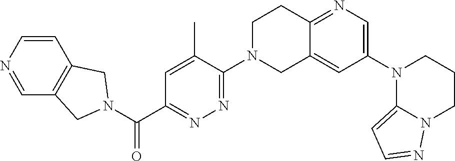 [SMILES: Cc1cc(C(=O)N2Cc3ccncc3C2)nnc1N1CCc2ncc(N3CCCn4nccc43)cc2C1] | 494.4 |
| 513 | (5,7-dihydro-6H-pyrrolo[3,4- b]pyridin-6-yl)(6-(3-(6,7- dihydropyrazolo[1,5-a]pyrimidin- 4(5H)-yl)-7,8-dihydro-1,6- naphthyridin-6(5H)-yl)-5- methylpyridazin-3-yl)methanone | 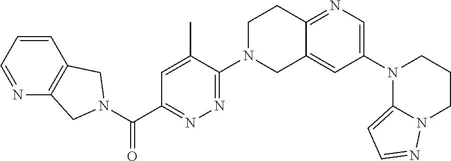 [SMILES: Cc1cc(C(=O)N2Cc3cccnc3C2)nnc1N1CCc2ncc(N3CCCn4nccc43)cc2C1] | 494.4 |
| 514 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(4- methylpiperazin-1-yl)methanone | 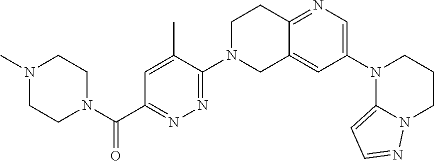 [SMILES: Cc1cc(C(=O)N2CCN(C)CC2)nnc1N1CCc2ncc(N3CCCn4nccc43)cc2C1] | 474.2 |
| 515 | (6-(3-(6,7-dihydropyrazolo[1,5- a]pyrimidin-4(5H)-yl)-7,8-dihydro- 1,6-naphthyridin-6(5H)-yl)-5- methylpyridazin-3- yl)(morpholino)methanone | 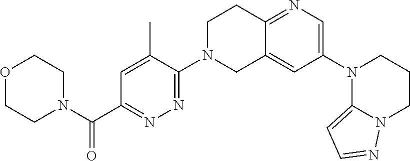 [SMILES: Cc1cc(C(=O)N2CCOCC2)nnc1N1CCc2ncc(N3CCCn4nccc43)cc2C1] | 461.2 |
| 516 | 5-methyl-6-(3-(1-methyl-1H- pyrazol-5-yl)-7,8-dihydro-1,6- naphthyridin-6(5H)-yl)-N-(thiazol- 5-ylmethyl)pyridazine-3- carboxamide | 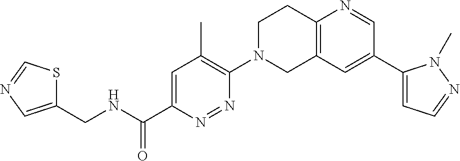 [SMILES: Cc1cc(C(=O)NCc2cncs2)nnc1N1CCc2ncc(-c3ccnn3C)cc2C1] | 447.2 |
| 517 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3-(pyridin-4- yl)azetidin-1-yl)methanone | 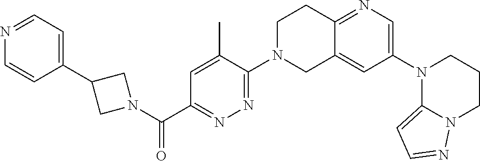 [SMILES: Cc1cc(C(=O)N2CC(c3ccncc3)C2)nnc1N1CCc2ncc(N3CCCn4nccc43)cc2C1] | 508.2 |
| 518 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3- (dimethylamino)azetidin-1- yl)methanone | 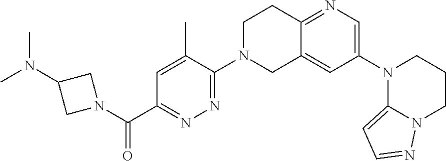 [SMILES: Cc1cc(C(=O)N2CC(N(C)C)C2)nnc1N1CCc2ncc(N3CCCn4nccc43)cc2C1] | 474.2 |
| 519 | 1-(6-(3-(6,7-dihydropyrazolo[1,5- a]pyrimidin-4(5H)-yl)-7,8-dihydro- 1,6-naphthyridin-6(5H)-yl)-5- methylpyridazine-3-carbonyl)-3- methylazetidine-3-carbonitrile | 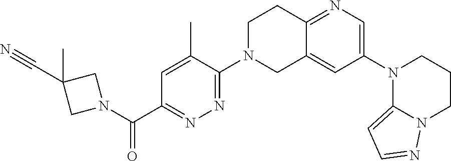 [SMILES: Cc1cc(C(=O)N2CC(C)(C#N)C2)nnc1N1CCc2ncc(N3CCCn4nccc43)cc2C1] | 470.2 |
| 520 | 6-(3-(6,7-dihydropyrazolo[1,5- a]pyrimidin-4(5H)-yl)-7,8-dihydro- 1,6-naphthyridin-6(5H)-yl)-5- methyl-N-(1-(2,2,2- trifluoroethyl)azetidin-3- yl)pyridazine-3-carboxamide | 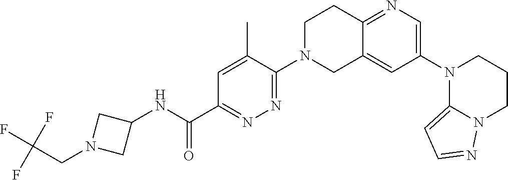 [SMILES: Cc1cc(C(=O)NC2CN(CC(F)(F)F)C2)nnc1N1CCc2ncc(N3CCCn4nccc43)cc2C1] | 528.2 |
| 521 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3-(pyridin-4- yl)pyrrolidin-1-yl)methanone | 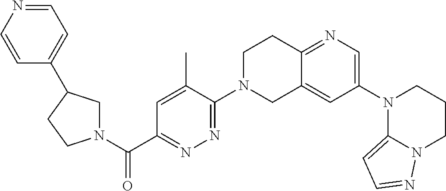 [SMILES: Cc1cc(C(=O)N2CCC(c3ccncc3)C2)nnc1N1CCc2ncc(N3CCCn4nccc43)cc2C1] | 522.2 |
| 522 | (3-(difluoromethyl)azetidin-1-yl)(5- methyl-6-(3-(trifluoromethyl)-7,8- dihydro-1,6-naphthyridin-6(5H)- yl)pyridazin-3-yl)methanone | 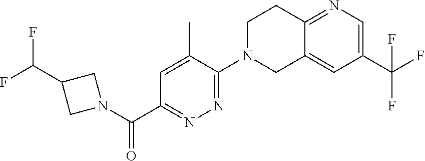 [SMILES: Cc1cc(C(=O)N2CC(C(F)F)C2)nnc1N1CCc2ncc(C(F)(F)F)cc2C1] | 428.4 |
| 523 | N-(1-cyanocyclopropyl)-5-methyl- 6-(3-(trifluoromethyl)-7,8-dihydro- 1,6-naphthyridin-6(5H)- yl)pyridazine-3-carboxamide | 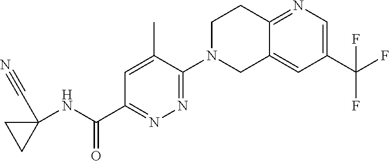 [SMILES: Cc1cc(C(=O)NC2(C#N)CC2)nnc1N1CCc2ncc(C(F)(F)F)cc2C1] | 403.4 |
| 524 | (3-(difluoromethyl)azetidin-1-yl)(6- (3-(6,7-dihydropyrazolo[1,5- a]pyrimidin-4(5H)-yl)-7,8-dihydro- 1,6-naphthyridin-6(5H)-yl)-5- methylpyridazin-3-yl)methanone | 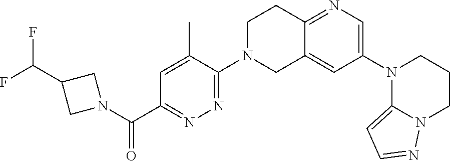 [SMILES: Cc1cc(C(=O)N2CC(C(F)F)C2)nnc1N1CCc2ncc(N3CCCn4nccc43)cc2C1] | 481.2 |
| 525 | N-(1-cyanocyclopropyl)-6-(3-(6,7- dihydropyrazolo[1,5-a]pyrimidin- 4(5H)-yl)-7,8-dihydro-1,6- naphthyridin-6(5H)-yl)-5- methylpyridazine-3-carboxamide | 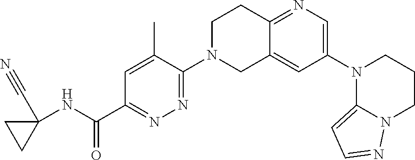 [SMILES: Cc1cc(C(=O)NC2(C#N)CC2)nnc1N1CCc2ncc(N3CCCn4nccc43)cc2C1] | 456.2 |
| 526 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3-(pyridin-3- yl)azetidin-1-yl)methanone | 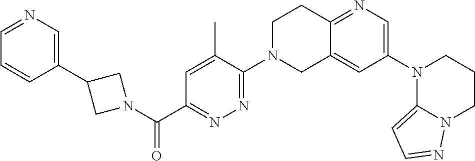 [SMILES: Cc1cc(C(=O)N2CC(c3cccnc3)C2)nnc1N1CCc2ncc(N3CCCn4nccc43)cc2C1] | 508.2 |
| 527 | (3-chloroazetidin-1-yl)(6-(3-(6,7- dihydropyrazolo[1,5-a]pyrimidin- 4(5H)-yl)-7,8-dihydro-1,6- naphthyridin-6(5H)-yl)-5- methylpyridazin-3-yl)methanone | 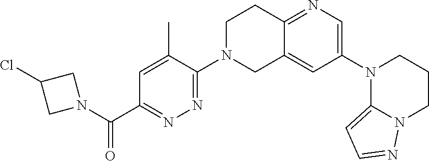 [SMILES: Cc1cc(C(=O)N2CC(Cl)C2)nnc1N1CCc2ncc(N3CCCn4nccc43)cc2C1] | 465.4 |
| 528 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3-(2- hydroxypropan-2-yl)azetidin-1- yl)methanone | 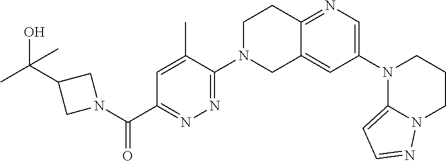 [SMILES: Cc1cc(C(=O)N2CC(C(C)(C)O)C2)nnc1N1CCc2ncc(N3CCCn4nccc43)cc2C1] | 489.5 |
| 529 | (6-(3-(6,7-dihydropyrazolo[1,5- a]pyrimidin-4(5H)-yl)-7,8-dihydro- 1,6-naphthyridin-6(5/I)-yl)-5- methylpyridazin-3-yl)(2,2- dimethylpyrrolidin-1-yl)methanone | 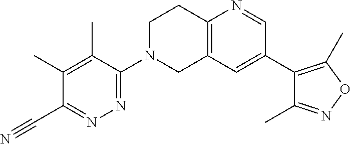 [SMILES: Cc1noc(C)c1-c1cnc2c(c1)CN(c1nnc(C#N)c(C)c1C)CC2] | 473.5 |
| 530 | (6-(3-(1H-pyrazol-1-yl)-7,8- dihydro-1,6-naphthyridin-6(5H)- yl)-5-methylpyridazin-3- yl)(pyrrolidin-1-yl)methanone | 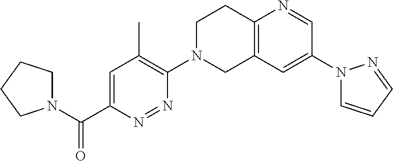 [SMILES: Cc1cc(C(=O)N2CCCC2)nnc1N1CCc2ncc(-n3cccn3)cc2C1] | 390.4 |
| 531 | (6-(3-(1H-pyrazol-1-yl)-7,8- dihydro-1,6-naphthyridin-6(5H)- yl)-5-methylpyridazin-3- yl)(azetidin-1-yl)methanone | 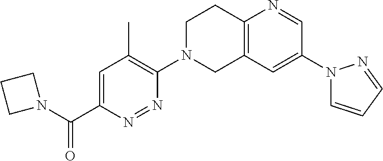 [SMILES: Cc1cc(C(=O)N2CCC2)nnc1N1CCc2ncc(-n3cccn3)cc2C1] | 376.4 |
| 532 | (6-(3-(1H-pyrazol-1-yl)-7,8- dihydro-1,6-naphthyridin-6(5H)- yl)-5-methylpyridazin-3-yl)(3- chloroazetidin-1-yl)methanone | 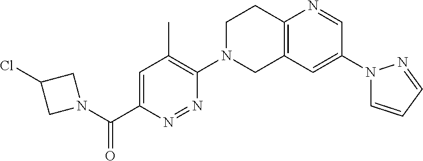 [SMILES: Cc1cc(C(=O)N2CC(Cl)C2)nnc1N1CCc2ncc(-n3cccn3)cc2C1] | 410.3 |
| 533 | (6-(3-(1H-pyrazol-1-yl)-7,8- dihydro-1,6-naphthyridin-6(5H)- yl)-5-methylpyridazin-3-yl)(3,3- difluoroazetidin-1-yl)methanone | 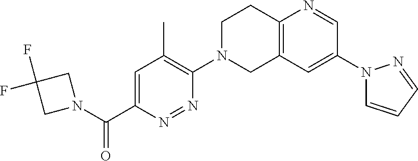 [SMILES: Cc1cc(C(=O)N2CC(F)(F)C2)nnc1N1CCc2ncc(-n3cccn3)cc2C1] | 412.4 |
| 534 | (6-(3-(1H-pyrazol-1-yl)-7,8- dihydro-1,6-naphthyridin-6(5H)- yl)-5-methylpyridazin-3-yl)(3- hydroxy-3-methylazetidin-1- yl)methanone | 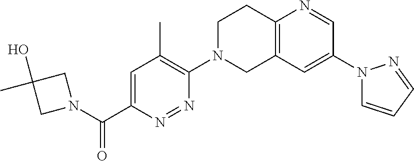 [SMILES: Cc1cc(C(=O)N2CC(C)(O)C2)nnc1N1CCc2ncc(-n3cccn3)cc2C1] | 406.4 |
| 535 | (6-(3-(1H-pyrazol-1-yl)-7,8- dihydro-1,6-naphthyridin-6(5H)- yl)-5-methylpyridazin-3-yl)(3- (pyridin-3-yl)azetidin-1- yl)methanone | 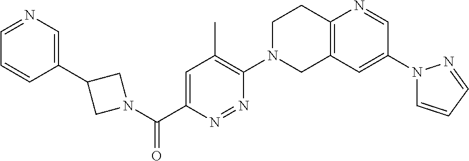 [SMILES: Cc1cc(C(=O)N2CC(c3cccnc3)C2)nnc1N1CCc2ncc(-n3cccn3)cc2C1] | 453.4 |
| 536 | (R)-(5-methyl-6-(3-(2- methylmorpholino)-7,8-dihydro- 1,6-naphthyridin-6(5H)- yl)pyridazin-3-yl)(pyrrolidin-1- yl)methanone | 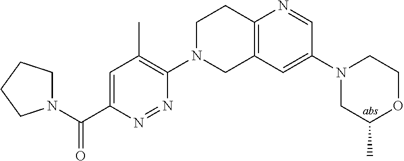 [SMILES: Cc1cc(C(=O)N2CCCC2)nnc1N1CCc2ncc(N3CCO[C@H](C)C3)cc2C1] | 423.4 |
| 537 | (R)-azetidin-1-yl(5-methyl-6-(3-(2- methylmorpholino)-7,8-dihydro- 1,6-naphthyridin-6(5H)- yl)pyridazin-3-yl)methanone | 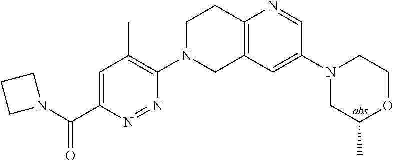 [SMILES: Cc1cc(C(=O)N2CCC2)nnc1N1CCc2ncc(N3CCO[C@H](C)C3)cc2C1] | 409.4 |
| 538 | (R)-(3-chloroazetidin-1-yl)(5- methyl-6-(3-(2-methylmorpholino)- 7,8-dihydro-1,6-naphthyridin- 6(5H)-yl)pyridazin-3-yl)methanone | 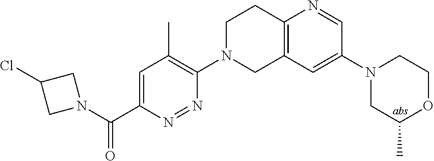 [SMILES: Cc1cc(C(=O)N2CC(Cl)C2)nnc1N1CCc2ncc(N3CCO[C@H](C)C3)cc2C1] | 443.3 |
| 539 | (R)-(3,3-difluoroazetidin-1-yl)(5- methyl-6-(3-(2-methylmorpholino)- 7,8-dihydro-1,6-naphthyridin- 6(5H)-yl)pyridazin-3-yl)methanone | 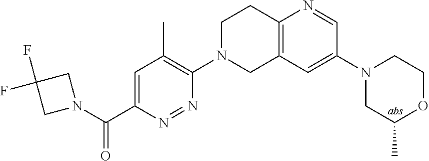 [SMILES: Cc1cc(C(=O)N2CC(F)(F)C2)nnc1N1CCc2ncc(N3CCO[C@H](C)C3)cc2C1] | 445.4 |
| 540 | (R)-(3-hydroxy-3-methylazetidin-1- yl)(5-methyl-6-(3-(2- methylmorpholino)-7,8-dihydro- 1,6-naphthyridin-6(5H)- yl)pyridazin-3-yl)methanone | 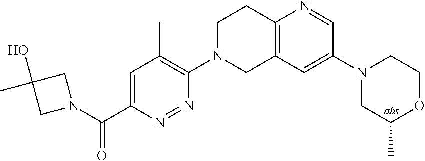 [SMILES: Cc1cc(C(=O)N2CC(C)(O)C2)nnc1N1CCc2ncc(N3CCO[C@H](C)C3)cc2C1] | 439.4 |
| 541 | (R)-(5-methyl-6-(3-(2- methylmorpholino)-7,8-dihydro- 1,6-naphthyridin-6(5H)- yl)pyridazin-3-yl)(3-(pyridin-3- yl)azetidin-1-yl)methanone | 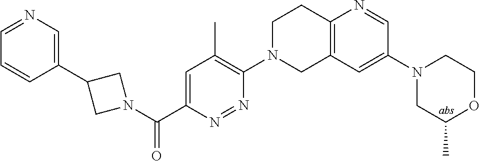 [SMILES: Cc1cc(C(=O)N2CC(c3cccnc3)C2)nnc1N1CCc2ncc(N3CCO[C@H](C)C3)cc2C1] | 486.4 |
| 542 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(2-oxa-6- azaspiro[3.3 ]heptan-6- yl)methanone | 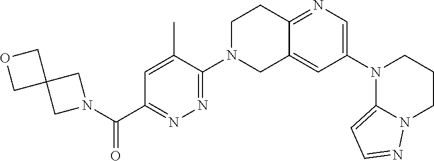 [SMILES: Cc1cc(C(=O)N2CC3(COC3)C2)nnc1N1CCc2ncc(N3CCCn4nccc43)cc2C1] | 473.4 |
| 543 | 6-(3-(6,7-dihydropyrazolo[1,5- a]pyrimidin-4(5H)-yl)-7,8-dihydro- 1,6-naphthyridin-6(5H)-yl)-5- methyl-N-(1-(pyridin-2- yl)cyclopropyl)pyridazine-3- carboxamide | 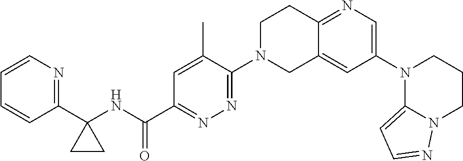 [SMILES: Cc1cc(C(=O)NC2(c3ccccn3)CC2)nnc1N1CCc2ncc(N3CCCn4nccc43)cc2C1] | 508.4 |
| 544 | (3,3-difluoroazetidin-1-yl)(6-(3- (6,7-dihydropyrazolo[1,5- a]pyrimidin-4(5H)-yl)-7,8-dihydro- 1,6-naphthyridin-6(5/)-yl)-5- methylpyridazin-3-yl)methanone | 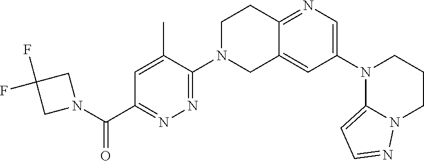 [SMILES: Cc1cc(C(=O)N2CC(F)(F)C2)nnc1N1CCc2ncc(N3CCCn4nccc43)cc2C1] | 467.4 |
| 545 | azetidin-1-yl(6-(3-(6,7- dihydropyrazolo[1,5-a]pyrimidin- 4(5H)-yl)-7,8-dihydro-1,6- naphthyridin-6(5H)-yl)-5- methylpyridazin-3-yl)methanone | 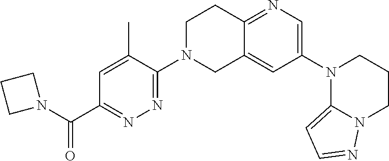 [SMILES: Cc1cc(C(=O)N2CCC2)nnc1N1CCc2ncc(N3CCCn4nccc43)cc2C1] | 431.4 |
| 546 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3-fluoro-3- methylazetidin-1-yl)methanone | 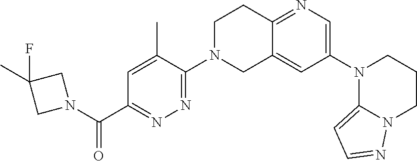 [SMILES: Cc1cc(C(=O)N2CC(C)(F)C2)nnc1N1CCc2ncc(N3CCCn4nccc43)cc2C1] | 463.3 |
| 547 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3-hydroxy- 3-methylazetidin-1-yl)methanone | 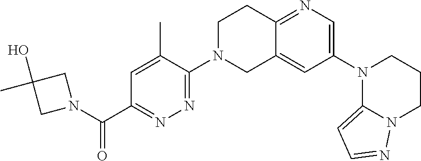 [SMILES: Cc1cc(C(=O)N2CC(C)(O)C2)nnc1N1CCc2ncc(N3CCCn4nccc43)cc2C1] | 461.4 |
| 548 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3- methoxyazetidin-1-yl)methanone | 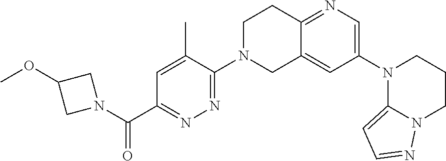 [SMILES: COC1CN(C(=O)c2cc(C)c(N3CCc4ncc(N5CCCn6nccc65)cc4C3)nn2)C1] | 461.4 |
| 549 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3- methylazetidin-1-yl)methanone | 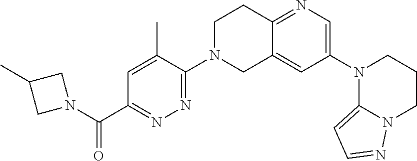 [SMILES: Cc1cc(C(=O)N2CC(C)C2)nnc1N1CCc2ncc(N3CCCn4nccc43)cc2C1] | 445.4 |
| 550 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(2- methylazetidin-1-yl)methanone | 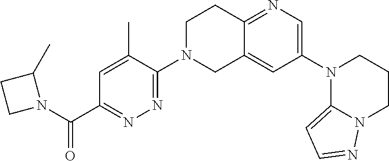 [SMILES: Cc1cc(C(=O)N2CCC2C)nnc1N1CCc2ncc(N3CCCn4nccc43)cc2C1] | 445.3 |
| 551 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3- fluoroazetidin-1-yl)methanone | 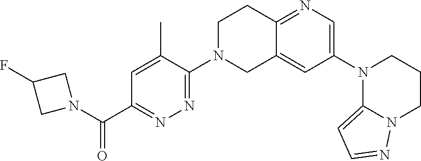 [SMILES: Cc1cc(C(=O)N2CC(F)C2)nnc1N1CCc2ncc(N3CCCn4nccc43)cc2C1] | 449.4 |
| 552 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3-methoxy- 3-methylazetidin-1-yl)methanone | 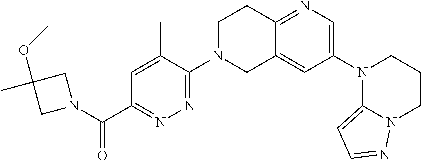 [SMILES: COC1(C)CN(C(=O)c2cc(C)c(N3CCc4ncc(N5CCCn6nccc65)cc4C3)nn2)C1] | 475.2 |
| 553 | 6-(3-(6,7-dihydropyrazolo[1,5- a]pyrimidin-4(5H)-yl)-7,8-dihydro- 1,6-naphthyridin-6(5H)-yl)-5- methyl-N-(1-(pyridin-3- yl)cyclopropyl)pyridazine-3- carboxamide | 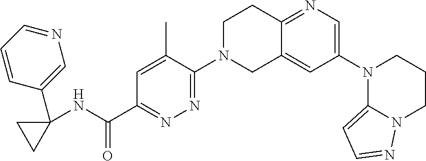 [SMILES: Cc1cc(C(=O)NC2(c3cccnc3)CC2)nnc1N1CCc2ncc(N3CCCn4nccc43)cc2C1] | 508.4 |
| 554 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(3- (hydroxymethyl)azetidin-1- yl)methanone | 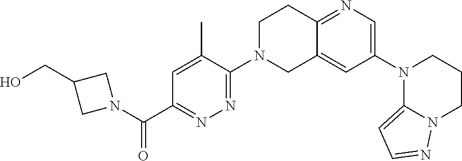 [SMILES: Cc1cc(C(=O)N2CC(CO)C2)nnc1N1CCc2ncc(N3CCCn4nccc43)cc2C1] | 461.4 |
| 555 | (6,6-difluoro-2- azaspiro[3.3]heptan-2-yl)(6-(3-(6,7- dihydropyrazolo[1,5-a]pyrimidin- 4(5H)-yl)-7,8-dihydro-1,6- naphthyridin-6(5H)-yl)-5- methylpyridazin-3-yl)methanone | 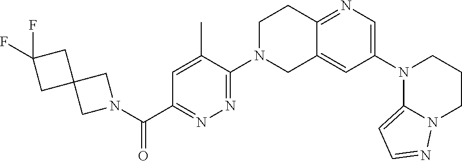 [SMILES: Cc1cc(C(=O)N2CC3(C2)CC(F)(F)C3)nnc1N1CCc2ncc(N3CCCn4nccc43)cc2C1] | 507.4 |
| 556 | 6-(3-(6,7-dihydropyrazolo[1,5- a]pyrimidin-4(5H)-yl)-7,8-dihydro- 1,6-naphthyridin-6(5H)-yl)-N,N,5- trimethylpyridazine-3-carboxamide | 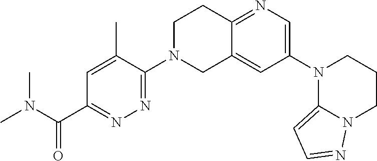 [SMILES: Cc1cc(C(=O)N(C)C)nnc1N1CCc2ncc(N3CCCn4nccc43)cc2C1] | 419.4 |
| 557 | (6-(3-(6,7-dihydropyrazolo[1,5- a]pyrimidin-4(5H)-yl)-7,8-dihydro- 1,6-naphthyridin-6(5H)-yl)-5- methylpyridazin-3-yl)(2- phenylazetidin-1-yl)methanone | 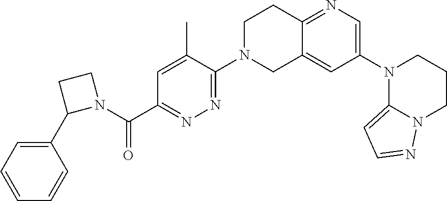 [SMILES: Cc1cc(C(=O)N2CCC2c2ccccc2)nnc1N1CCc2ncc(N3CCCn4nccc43)cc2C1] | 507.3 |
| 558 | (3-(difluoromethyl)azetidin-1-yl)(5- methyl-6-(3-((1-methyl-1H- pyrazol-5-yl)amino)-7,8-dihydro- 1,6-naphthyridin-6(5H)- yl)pyridazin-3-yl)methanone | 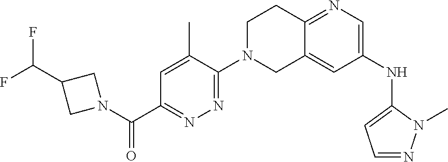 [SMILES: Cc1cc(C(=O)N2CC(C(F)F)C2)nnc1N1CCc2ncc(Nc3ccnn3C)cc2C1] | 455.4 |
| 559 | (3-fluoroazetidin-1-yl)(5-methyl-6- (3-((1-methyl-1H-pyrazol-5- yl)amino)-7,8-dihydro-1,6- naphthyridin-6(5H)-yl)pyridazin-3- yl)methanone | 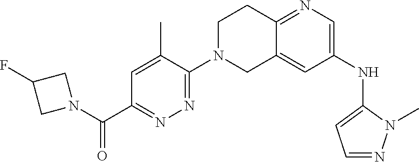 [SMILES: Cc1cc(C(=O)N2CC(F)C2)nnc1N1CCc2ncc(Nc3ccnn3C)cc2C1] | 423.3 |
| 560 | (3-fluoro-3-methylazetidin-1-yl)(5- methyl-6-(3-((1-methyl-1H- pyrazol-5-yl)amino)-7,8-dihydro- 1,6-naphthyridin-6(5H)- yl)pyridazin-3-yl)methanone | 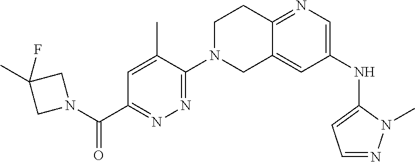 | 437.3 |
| 561 | (5-methyl-6-(3-((1-methyl-1H- pyrazol-5-yl)amino)-7,8-dihydro- 1,6-naphthyridin-6(5H)- yl)pyridazin-3-yl)(2-oxa-6- azaspiro[3.3 ]heptan-6- yl)methanone | 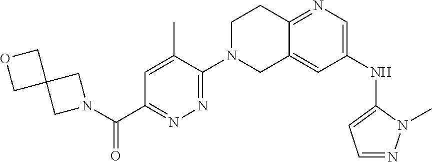 [SMILES: Cc1cc(C(=O)N2CC3(COC3)C2)nnc1N1CCc2ncc(Nc3ccnn3C)cc2C1] | 447.4 |
| 562 | (3-(difluoromethyl)azetidin-1-yl)(5- methyl-6-(3-(1-methyl-1H-pyrazol- 5-yl)-7,8-dihydro-1,6-naphthyridin- 6(5H)-yl)pyridazin-3-yl)methanone | 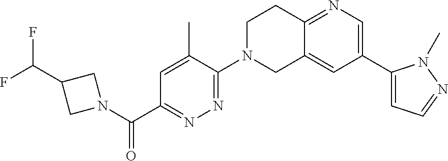 [SMILES: Cc1cc(C(=O)N2CC(C(F)F)C2)nnc1N1CCc2ncc(-c3ccnn3C)cc2C1] | 440.3 |
| 563 | (3-fluoroazetidin-1-yl)(5-methyl-6- (3-(1-methyl-1H-pyrazol-5-yl)-7,8- dihydro-1,6-naphthyridin-6(5H)- yl)pyridazin-3-yl)methanone | 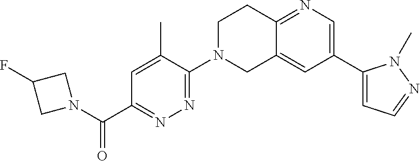 [SMILES: Cc1cc(C(=O)N2CC(F)C2)nnc1N1CCc2ncc(-c3ccnn3C)cc2C1] | 408.3 |
| 564 | (3-fluoro-3-methylazetidin-1-yl)(5- methyl-6-(3-(1-methyl-1H-pyrazol- 5-yl)-7,8-dihydro-1,6-naphthyridin- 6(5H)-yl)pyridazin-3-yl)methanone | 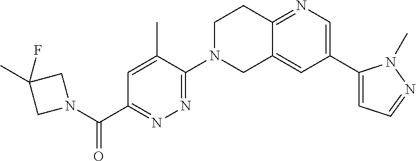 [SMILES: Cc1cc(C(=O)N2CC(C)(F)C2)nnc1N1CCc2ncc(-c3ccnn3C)cc2C1] | 422.3 |
| 565 | (5-methyl-6-(3-(1-methyl-1H- pyrazol-5-yl)-7,8-dihydro-1,6- naphthyridin-6(5H)-yl)pyridazin-3- yl)(2-oxa-6-azaspiro[3.3]heptan-6- yl)methanone | 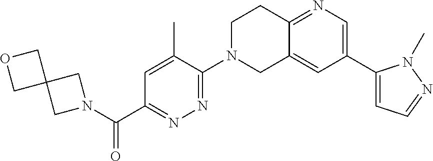 [SMILES: Cc1cc(C(=O)N2CC3(COC3)C2)nnc1N1CCc2ncc(-c3ccnn3C)cc2C1] | 432.4 |
| 566 | (3,3-difluoroazetidin-1-yl)(5- methyl-6-(3-((1-methyl-1H- pyrazol-5-yl)amino)-7,8-dihydro- 1,6-naphthyridin-6(5H)- yl)pyridazin-3-yl)methanone | 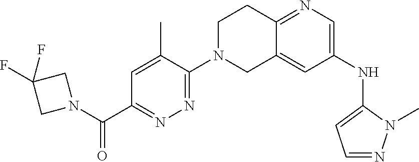 [SMILES: Cc1cc(C(=O)N2CC(F)(F)C2)nnc1N1CCc2ncc(Nc3ccnn3C)cc2C1] | 441.3 |
| 567 | (3,3-difluoroazetidin-1-yl)(5- methyl-6-(3-(1-methyl-1H-pyrazol- 5-yl)-7,8-dihydro-1,6-naphthyridin- 6(5H)-yl)pyridazin-3-yl)methanone | 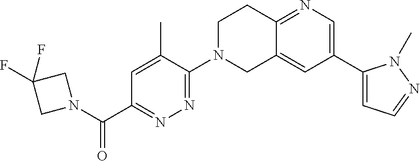 [SMILES: Cc1cc(C(=O)N2CC(F)(F)C2)nnc1N1CCc2ncc(-c3ccnn3C)cc2C1] | 426.4 |
| 568 | (6-(3-(1H-pyrazol-1-yl)-7,8- dihydro-1,6-naphthyridin-6(5H)- yl)-5-methylpyridazin-3-yl)(3- fluoroazetidin-1-yl)methanone | 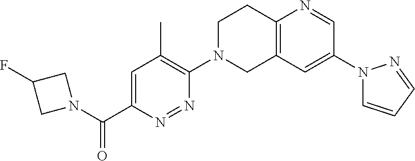 [SMILES: Cc1cc(C(=O)N2CC(F)C2)nnc1N1CCc2ncc(-n3cccn3)cc2C1] | 394.3 |
| 569 | (6-(3-(1H-pyrazol-1-yl)-7,8- dihydro-1,6-naphthyridin-6(5H)- yl)-5-methylpyridazin-3-yl)(3- fluoro-3-methylazetidin-1- yl)methanone | 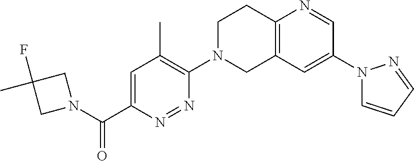 [SMILES: Cc1cc(C(=O)N2CC(C)(F)C2)nnc1N1CCc2ncc(-n3cccn3)cc2C1] | 408.4 |
| 570 | (6-(3-(1H-pyrazol-1-yl)-7,8- dihydro-1,6-naphthyridin-6(5H)- yl)-5-methylpyridazin-3-yl)(6,6- difluoro-2-azaspiro[3.3 ]heptan-2- yl)methanone | 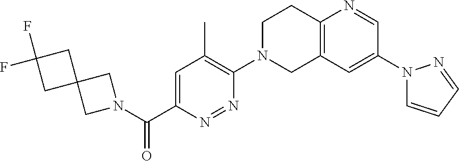 [SMILES: Cc1cc(C(=O)N2CC3(C2)CC(F)(F)C3)nnc1N1CCc2ncc(-n3cccn3)cc2C1] | 452.4 |
| 571 | (6-(3-(1H-pyrazol-1-yl)-7,8- dihydro-1,6-naphthyridin-6(5H)- yl)-5-methylpyridazin-3-yl)(2-oxa- 6-azaspiro[3.3]heptan-6- yl)methanone | 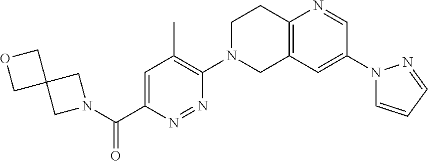 [SMILES: Cc1cc(C(=O)N2CC3(COC3)C2)nnc1N1CCc2ncc(-n3cccn3)cc2C1] | 418.4 |
| 572 | (R)-(3-fluoroazetidin-1-yl)(5- methyl-6-(3-(2-methylmorpholino)- 7,8-dihydro-1,6-naphthyridin- 6(5H)-yl)pyridazin-3-yl)methanone | 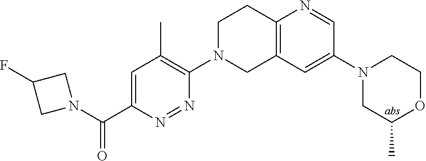 [SMILES: Cc1cc(C(=O)N2CC(F)C2)nnc1N1CCc2ncc(N3CCO[C@H](C)C3)cc2C1] | 427.3 |
| 573 | (R)-(3-fluoro-3-methylazetidin-1- yl)(5-methyl-6-(3-(2- methylmorpholino)-7,8-dihydro- 1,6-naphthyridin-6(5H)- yl)pyridazin-3-yl)methanone | 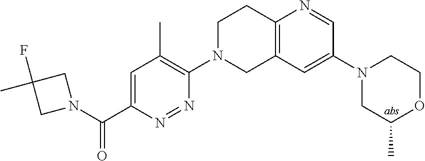 [SMILES: Cc1cc(C(=O)N2CC(C)(F)C2)nnc1N1CCc2ncc(N3CCO[C@H](C)C3)cc2C1] | 441.4 |
| 574 | (R)-(6,6-difluoro-2- azaspiro[3.3 ]heptan-2-yl)(5-methyl- 6-(3-(2-methylmorpholino)-7,8- dihydro-1,6-naphthyridin-6(5H)- yl)pyridazin-3-yl)methanone | 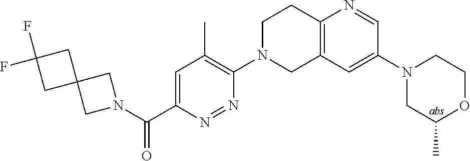 [SMILES: Cc1cc(C(=O)N2CC3(C2)CC(F)(F)C3)nnc1N1CCc2ncc(N3CCO[C@H](C)C3)cc2C1] | 485.4 |
| 575 | (R)-(5-methyl-6-(3-(2- methylmorpholino)-7,8-dihydro- 1,6-naphthyridin-6(5H)- yl)pyridazin-3-yl)(2-oxa-6- azaspiro[3.3 ]heptan-6- yl)methanone | 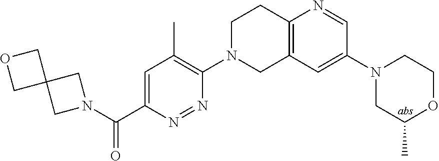 [SMILES: Cc1cc(C(=O)N2CC3(COC3)C2)nnc1N1CCc2ncc(N3CCO[C@H](C)C3)cc2C1] | 451.4 |
| 576 | (3,3-difluoroazetidin-1-yl)(5- methyl-6-(3-(methyl-d₃)-7,8- dihydro-1,6-naphthyridin-6(5H)- yl)pyridazin-3-yl)methanone | 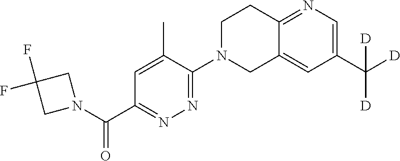 [SMILES: [2H]C([2H])([2H])c1cnc2c(c1)CN(c1nnc(C(=O)N3CC(F)(F)C3)cc1C)CC2] | 363.3 |
| 577 | (5-methyl-6-(3-(methyl-d₃)-7,8- dihydro-1,6-naphthyridin-6(5H)- yl)pyridazin-3-yl)(2-oxa-6- azaspiro[3.3 ]heptan-6- yl)methanone | 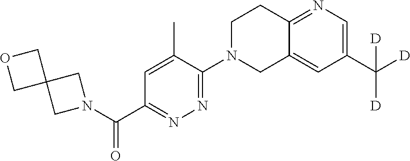 [SMILES: [2H]C([2H])([2H])c1cnc2c(c1)CN(c1nnc(C(=O)N3CC4(COC4)C3)cc1C)CC2] | 369.4 |
| 578 | (5-methyl-6-(3-(methyl- d₃)-7,8- dihydro-1,6-naphthyridin-6(5H)- yl)pyridazin-3-yl)(pyrrolidin-1- yl)methanone | 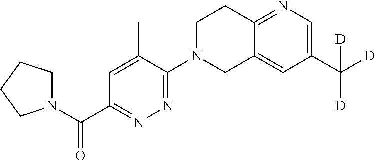 [SMILES: [2H]C([2H])([2H])c1cnc2c(c1)CN(c1nnc(C(=O)N3CCCC3)cc1C)CC2] | 341.4 |
| 579 | 5-methyl-6-(3-(methyl- d₃)-7,8- dihydro-1,6-naphthyridin-6(5H)- yl)-N-(pyridin-4- ylmethyl)pyridazine-3-carboxamide | 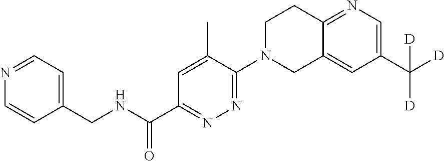 [SMILES: [2H]C([2H])([2H])c1cnc2c(c1)CN(c1nnc(C(=O)NCc3ccncc3)cc1C)CC2] | 378.4 |
| 580 | 5-methyl-6-(3-(methyl- d₃)-7,8- dihydro-1,6-naphthyridin-6(5H)- yl)-N-(1-(pyridin-3- yl)cyclopropyl)pyridazine-3- carboxamide | 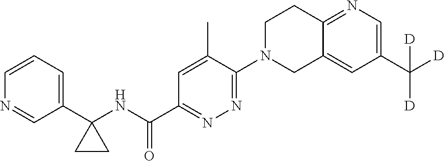 [SMILES: [2H]C([2H])([2H])c1cnc2c(c1)CN(c1nnc(C(=O)NC3(c4cccnc4)CC3)cc1C)CC2] | 404.4 |
| 581 | 5-methyl-N-(pyridin-4-ylmethyl)-6- (3-(2,2,2-trifluoroethyl)-7,8- dihydro-1,6-naphthyridin-6(5H)- yl)pyridazine-3-carboxamide | 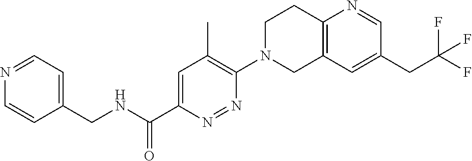 [SMILES: Cc1cc(C(=O)NCc2ccncc2)nnc1N1CCc2ncc(CC(F)(F)F)cc2C1] | 443.4 |
| 582 | (5-methyl-6-(3-(2,2,2- trifluoroethyl)-7,8-dihydro-1,6- naphthyridin-6(5H)-yl)pyridazin-3- yl)(pyrrolidin-1-yl)methanone | 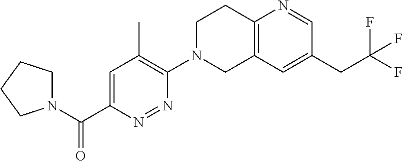 [SMILES: Cc1cc(C(=O)N2CCCC2)nnc1N1CCc2ncc(CC(F)(F)F)cc2C1] | 406.4 |
| 583 | (3,3-difluoroazetidin-1-yl)(5- methyl-6-(3-(2,2,2-trifluoroethyl)- 7,8-dihydro-1,6-naphthyridin- 6(5H)-yl)pyridazin-3-yl)methanone | 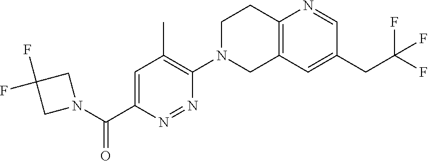 [SMILES: Cc1cc(C(=O)N2CC(F)(F)C2)nnc1N1CCc2ncc(CC(F)(F)F)cc2C1] | 426.4 |
| 584 | (3,3-bis(hydroxymethyl)azetidin-1- yl)(5-methyl-6-(3-(2,2,2- trifluoroethyl)-7,8-dihydro-1,6- naphthyridin-6(5H)-yl)pyridazin-3- yl)methanone | 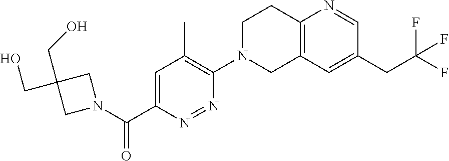 [SMILES: Cc1cc(C(=O)N2CC(CO)(CO)C2)nnc1N1CCc2ncc(CC(F)(F)F)cc2C1] | 452.3 |
| 585 | 5-methyl-N-(1-(pyridin-3- yl)cyclopropyl)-6-(3-(2,2,2- trifluoroethyl)-7,8-dihydro-1,6- naphthyridin-6(5H)-yl)pyridazine- 3-carboxamide | 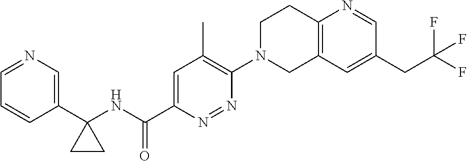 [SMILES: Cc1cc(C(=O)NC2(c3cccnc3)CC2)nnc1N1CCc2ncc(CC(F)(F)F)cc2C1] | 469.4 |
| 586 | (3-fluoroazetidin-1-yl)(5-methyl-6- (3-(2,2,2-trifluoroethyl)-7,8- dihydro-1,6-naphthyridin-6(5H)- yl)pyridazin-3-yl)methanone | 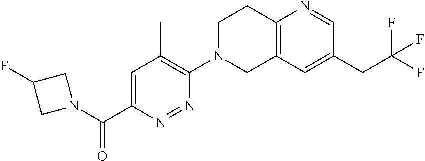 [SMILES: Cc1cc(C(=O)N2CC(F)C2)nnc1N1CCc2ncc(CC(F)(F)F)cc2C1] | 410.4 |
| 587 | (5-methyl-6-(3-(2,2,2- trifluoroethyl)-7,8-dihydro-1,6- naphthyridin-6(5/)-yl)pyridazin-3- yl)(morpholino)methanone | 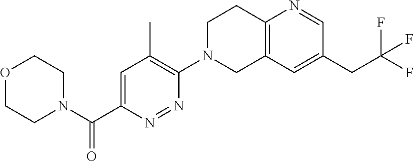 [SMILES: Cc1cc(C(=O)N2CCOCC2)nnc1N1CCc2ncc(CC(F)(F)F)cc2C1] | 422.4 |
| 588 | (3-hydroxy-3-methylazetidin-1- yl)(5-methyl-6-(3-(2,2,2- trifluoroethyl)-7,8-dihydro-1,6- naphthyridin-6(5H)-yl)pyridazin-3- yl)methanone | 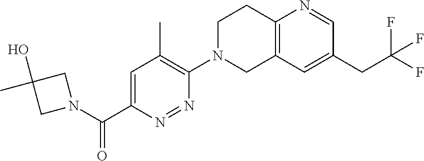 [SMILES: Cc1cc(C(=O)N2CC(C)(O)C2)nnc1N1CCc2ncc(CC(F)(F)F)cc2C1] | 422.4 |
| 589 | (3-methoxyazetidin-1-yl)(5-methyl- 6-(3-(2,2,2-trifluoroethyl)-7,8- dihydro-1,6-naphthyridin-6(5H)- yl)pyridazin-3-yl)methanone | 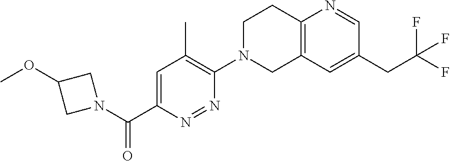 [SMILES: COC1CN(C(=O)c2cc(C)c(N3CCc4ncc(CC(F)(F)F)cc4C3)nn2)C1] | 422.4 |
| 590 | 5-methyl-N-(thiazol-5-ylmethyl)-6- (3-(2,2,2-trifluoroethyl)-7,8- dihydro-1,6-naphthyridin-6(5H)- yl)pyridazine-3-carboxamide | 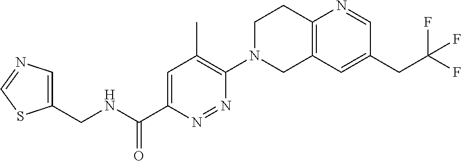 [SMILES: Cc1cc(C(=O)NCc2cncs2)nnc1N1CCc2ncc(CC(F)(F)F)cc2C1] | 449.4 |
| 591 | 4-benzyl-7-bromo-8-(3- (trifluoromethyl)-7,8-dihydro-1,6- naphthyridin-6(5H)-yl)-3,4- dihydropyrido[3,2-f][1,4]oxazepin- 5(2H)-one | 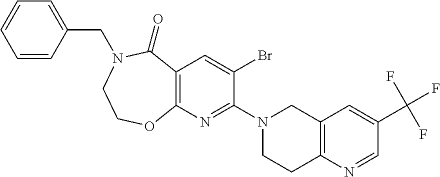 [SMILES: O=C1c2cc(Br)c(N3CCc4ncc(C(F)(F)F)cc4C3)nc2OCCN1Cc1ccccc1] | 533 |
| 592 | 4-benzyl-7-methyl-8-(3- (trifluoromethyl)-7,8-dihydro-1,6- naphthyridin-6(5H)-yl)-3,4- dihydropyrido[3,2-f][1,4]oxazepin- 5(2H)-one | 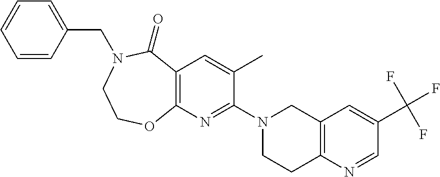 [SMILES: Cc1cc2c(nc1N1CCc3ncc(C(F)(F)F)cc3C1)OCCN(Cc1ccccc1)C2=O] | 469 |
| 593 | 6-(3-(6-fluoro-4-methylpyridin-3- yl)-7,8-dihydro-1,6-naphthyridin- 6(5H)-yl)-5-methylpyridazine-3- carbonitrile | 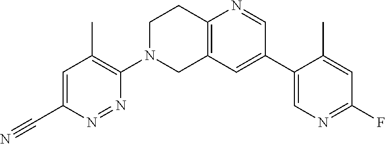 [SMILES: Cc1cc(F)ncc1-c1cnc2c(c1)CN(c1nnc(C#N)cc1C)CC2] | 361 |
| 594 | 6-(3-bromo-7,8-dihydro-1,6- naphthyridin-6(5H)-yl)-5- methylnicotinonitrile | 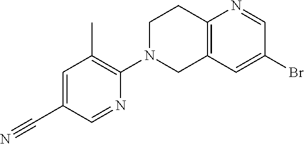 [SMILES: Cc1cc(C#N)cnc1N1CCc2ncc(Br)cc2C1] | 329.1 |
| 595 | 6-(3-(4,5-difluoro-2-methylphenyl)- 7,8-dihydro-1,6-naphthyridin- 6(5H)-yl)-5-methylnicotinonitrile | 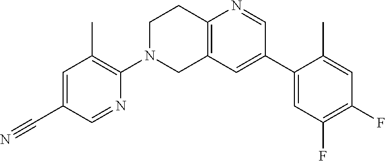 [SMILES: Cc1cc(F)c(F)cc1-c1cnc2c(c1)CN(c1ncc(C#N)cc1C)CC2] | 377.3 |
| 596 | 6-(3-(4,5-difluoro-2-methylphenyl)- 7,8-dihydro-1,6-naphthyridin- 6(5H)-yl)-5-methylpyridazine-3- carbonitrile | 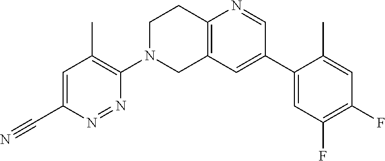 [SMILES: Cc1cc(F)c(F)cc1-c1cnc2c(c1)CN(c1nnc(C#N)cc1C)CC2] | 378.2 |
| 597 | 6-(3-(3,5-difluoro-2-methylphenyl)- 7,8-dihydro-1,6-naphthyridin- 6(5H)-yl)-5-methylnicotinonitrile | 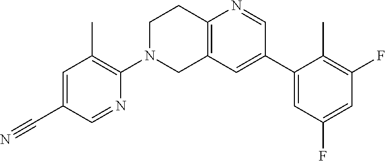 [SMILES: Cc1cc(C#N)cnc1N1CCc2ncc(-c3cc(F)cc(F)c3C)cc2C1] | 377.2 |
| 598 | 6-(3-((3-fluoropyridin-4-yl)amino)- 7,8-dihydro-1,6-naphthyridin- 6(5H)-yl)-5-methylpyridazine-3- carbonitrile | 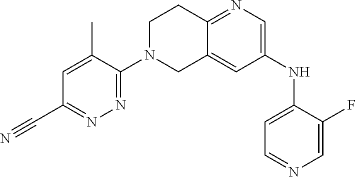 [SMILES: Cc1cc(C#N)nnc1N1CCc2ncc(Nc3ccncc3F)cc2C1] | 362 |
| 599 | 6-(3-((3-fluoropyridin-4-yl)amino)- 7,8-dihydro-1,6-naphthyridin- 6(5H)-yl)-5-methylnicotinonitrile | 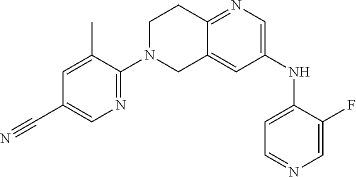 [SMILES: Cc1cc(C#N)cnc1N1CCc2ncc(Nc3ccncc3F)cc2C1] | 361 |
| 600 | 6-(3-((5-fluoro-2-methylpyridin-4- yl)amino)-7,8-dihydro-1,6- naphthyridin-6(5H)-yl-5,5,7,7-d4)- 4,5-dimethylpyridazine-3- carbonitrile | 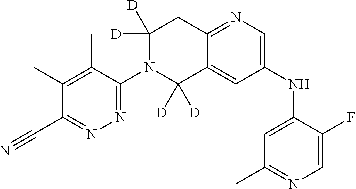 [SMILES: [2H]C1([2H])Cc2ncc(Nc3cc(C)ncc3F)cc2C([2H])([2H])N1c1nnc(C#N)c(C)c1C] | 394 |
| 601 | 6-(3-((5-fluoro-2-methylpyridin-4- yl)amino)-7,8-dihydro-1,6- naphthyridin-6(5H)-yl-5,5,7,7-d₄)- 5-methylnicotinonitrile | 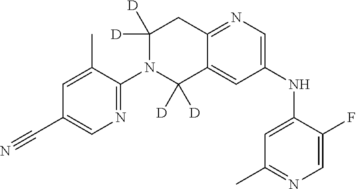 [SMILES: [2H]C1([2H])Cc2ncc(Nc3cc(C)ncc3F)cc2C([2H])([2H])N1c1ncc(C#N)cc1C] | 379 |
| 602 | 5-methyl-6-(3-((2-methylpyridin-3- yl)amino)-7,8-dihydro-1,6- naphthyridin-6(5H)-yl-5,5,7,7- d₄)nicotinonitrile | 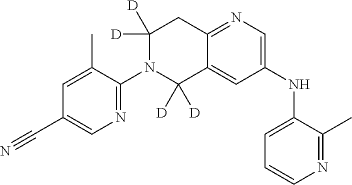 [SMILES: [2H]C1([2H])Cc2ncc(Nc3cccnc3C)cc2C([2H])([2H])N1c1ncc(C#N)cc1C] | 361 |
| 603 | 6-(3-((5-fluoro-2-methylpyridin-4- yl)amino)-7,8-dihydro-1,6- naphthyridin-6(5H)-yl-5,5,7,7-d₄)- 5-methylpyridazine-3-carbonitrile | 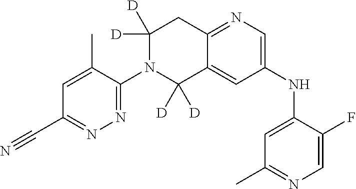 [SMILES: [2H]C1([2H])Cc2ncc(Nc3cc(C)ncc3F)cc2C([2H])([2H])N1c1nnc(C#N)cc1C] | 380 |
| 604 | 5-methyl-6-(3-((2-methylpyridin-3- yl)amino)-7,8-dihydro-1,6- naphthyridin-6(5H)-yl-5,5,7,7- d₄)pyridazine-3-carbonitrile | 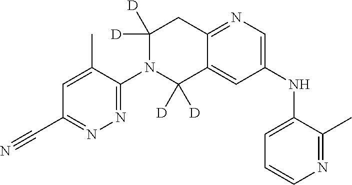 [SMILES: [2H]C1([2H])Cc2ncc(Nc3cccnc3C)cc2C([2H])([2H])N1c1nnc(C#N)cc1C] | 362 |
| 605 | 5-methyl-6-(2-methyl-7,8-dihydro- 1,6-naphthyridin-6(5H)- yl)pyridazine-3-carbonitrile | 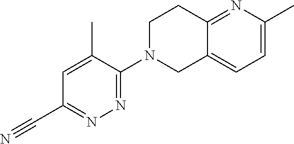 [SMILES: Cc1ccc2c(n1)CCN(c1nnc(C#N)cc1C)C2] | 266 |
| 606 | 6-(3-((3-fluoropyridin-4-yl)amino)- 7,8-dihydro-1,6-naphthyridin- 6(5H)-yl-5,5,7,7-d4)-5- methylpyridazine-3-carbonitrile | 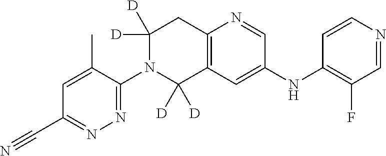 [SMILES: [2H]C1([2H])Cc2ncc(Nc3ccncc3F)cc2C([2H])([2H])N1c1nnc(C#N)cc1C] | 366 |
| 607 | 5-methyl-6-(3-((3-methylpyridin-4- yl)amino)-7,8-dihydro-1,6- naphthyridin-6(5H)-yl-5,5,7,7- d₄)pyridazine-3-carbonitrile | 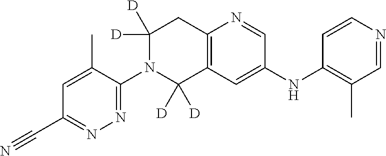 [SMILES: [2H]C1([2H])Cc2ncc(Nc3ccncc3C)cc2C([2H])([2H])N1c1nnc(C#N)cc1C] | 362 |
| 608 | 6-(3-((3-fluoropyridin-4-yl)amino)- 7,8-dihydro-1,6-naphthyridin- 6(5H)-yl-5,5,7,7-d4)-4,5- dimethylpyridazine-3-carbonitrile | 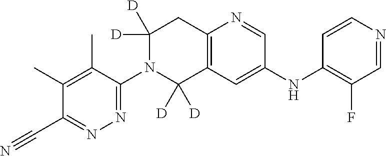 [SMILES: [2H]C1([2H])Cc2ncc(Nc3ccncc3F)cc2C([2H])([2H])N1c1nnc(C#N)c(C)c1C] | 380 |
| 609 | 4,5-dimethyl-6-(3-((3- methylpyridin-4-yl)amino)-7,8- dihydro-1,6-naphthyridin-6(5H)-yl- 5,5,7,7-d₄)pyridazine-3-carbonitrile | 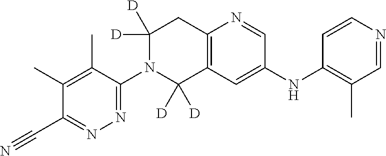 [SMILES: [2H]C1([2H])Cc2ncc(Nc3ccncc3C)cc2C([2H])([2H])N1c1nnc(C#N)c(C)c1C] | 376 |
| 610 | 5-methyl-6-(3-((3-methylpyridin-4- yl)amino)-7,8-dihydro-1,6- naphthyridin-6(5H)-yl-5,5,7,7- d₄)nicotinonitrile | 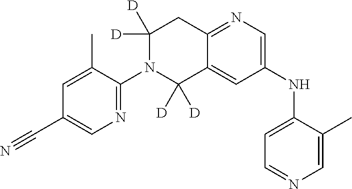 [SMILES: [2H]C1([2H])Cc2ncc(Nc3ccncc3C)cc2C([2H])([2H])N1c1ncc(C#N)cc1C] | 361 |
| 611 | 6-(3-((2-fluorophenyl)amino)-7,8- dihydro-1,6-naphthyridin-6(5H)-yl- 5,5,7,7-d₄)-5-methylpyridazine-3- carbonitrile | 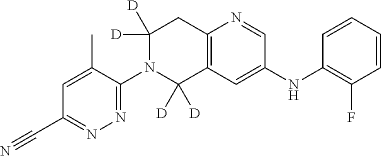 [SMILES: [2H]C1([2H])Cc2ncc(Nc3ccccc3F)cc2C([2H])([2H])N1c1nnc(C#N)cc1C] | 365 |
| 612 | 6-(3-((2-fluorophenyl)amino)-7,8- dihydro-1,6-naphthyridin-6(5H)-yl- 5,5,7,7-d₄)-5-methylnicotinonitrile | 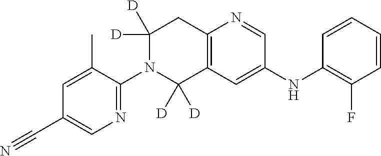 [SMILES: [2H]C1([2H])Cc2ncc(Nc3ccccc3F)cc2C([2H])([2H])N1c1ncc(C#N)cc1C] | 364 |
| 613 | 6-(3-((3-fluoropyridin-4-yl)amino)- 7,8-dihydro-1,6-naphthyridin- 6(5H)-yl)-4,5-dimethylpyridazine- 3-carbonitrile | 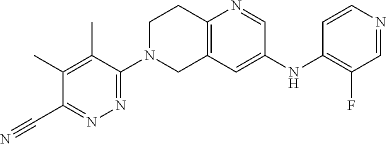 [SMILES: Cc1c(C#N)nnc(N2CCc3ncc(Nc4ccncc4F)cc3C2)c1C] | 376 |
| 614 | 6-(3-((5-fluoro-2-methylpyridin-4- yl)amino)-7,8-dihydro-1,6- naphthyridin-6(5H)-yl)-4,5- dimethylpyridazine-3-carbonitrile | 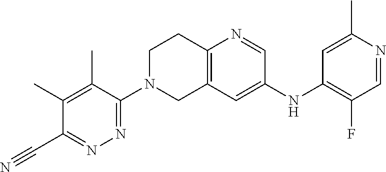 [SMILES: Cc1cc(Nc2cnc3c(c2)CN(c2nnc(C#N)c(C)c2C)CC3)c(F)cn1] | 390 |
| 615 | 6-(3-((5-fluoro-2-methylpyridin-4- yl)amino)-7,8-dihydro-1,6- naphthyridin-6(5H)-yl)-5- methylpyridazine-3-carbonitrile | 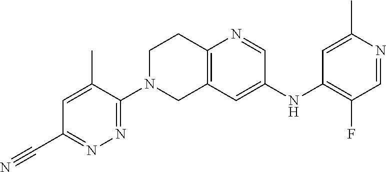 [SMILES: Cc1cc(Nc2cnc3c(c2)CN(c2nnc(C#N)cc2C)CC3)c(F)cn1] | 376 |
| 616 | 6-(3-((5-fluoro-2-methylpyridin-4- yl)amino)-7,8-dihydro-1,6- naphthyridin-6(5H)-yl)-5- methylnicotinonitrile | 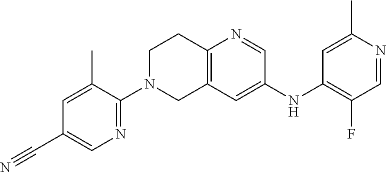 [SMILES: Cc1cc(Nc2cnc3c(c2)CN(c2ncc(C#N)cc2C)CC3)c(F)cn1] | 375 |
| 617 | 5-methyl-6-(3-(1-methyl-1H- pyrazol-5-yl)-7,8-dihydro-1,6- naphthyridin-6(5H)-yl-5,5,7,7- d₄)pyridazine-3-carbonitrile | 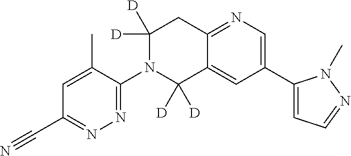 [SMILES: [2H]C1([2H])Cc2ncc(-c3ccnn3C)cc2C([2H])([2H])N1c1nnc(C#N)cc1C] | 336 |
| 618 | 4,5-dimethyl-6-(3-((3- methylpyridin-4-yl)amino)-7,8- dihydro-1,6-naphthyridin-6(5)- yl)pyridazine-3-carbonitrile | 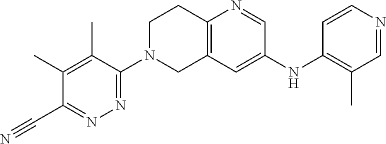 [SMILES: Cc1cnccc1Nc1cnc2c(c1)CN(c1nnc(C#N)c(C)c1C)CC2] | 372 |
| 619 | 5-methyl-6-(3-((3-methylpyridin-4- yl)amino)-7,8-dihydro-1,6- naphthyridin-6(5H)-yl)pyridazine- 3-carbonitrile | 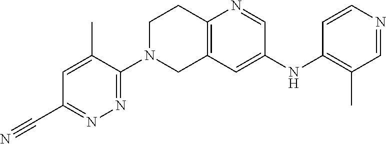 [SMILES: Cc1cnccc1Nc1cnc2c(c1)CN(c1nnc(C#N)cc1C)CC2] | 358 |
| 620 | 5-methyl-6-(3-((3-methylpyridin-4- yl)amino)-7,8-dihydro-1,6- naphthyridin-6(5H)- yl)nicotinonitrile | 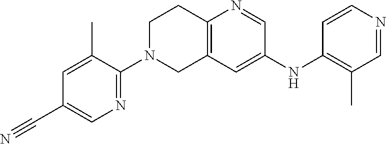 [SMILES: Cc1cnccc1Nc1cnc2c(c1)CN(c1ncc(C#N)cc1C)CC2] | 357 |
| 621 | 6-(3-(3,5-dimethylisoxazol-4-yl)- 7,8-dihydro-1,6-naphthyridin- 6(5H)-yl-5,5,7,7-d4)-5- methylpyridazine-3-carbonitrile | 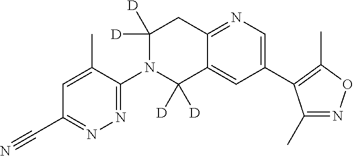 [SMILES: [2H]C1([2H])Cc2ncc(-c3c(C)noc3C)cc2C([2H])([2H])N1c1nnc(C#N)cc1C] | 351 |

^{a}Prepared from the first eluting enantiomer by chiral SFC separation in Example 36, Step B.
^{b}Prepared from the second eluting enantiomer by chiral SFC separation in Example 36, Step B.

### Biological Activity

### A. Cell Lines Expressing Muscarinic Acetylcholine Receptors

Human and rat M₄ cDNAs, along with the chimeric G protein G_{qi5}, were transfected into Chinese hamster ovary (CHO-K1) cells purchased from the American Type Culture Collection using Lipofectamine2000. hM₄-G_{qi5} cells were grown in Ham's F-12 medium containing 10% heat-inactivated fetal bovine serum (FBS), 20 mM HEPES, 50 μg/mL G418 sulfate, and 500 μg/mL Hygromycin B. rM₄-G_{gi5} cells were grown in DiVEM containing 10% heat-inactivated FBS, 20 mM THEPES, 400 μμg/mL G418 sulfate, and 500 μμg/mL Hygromycin B.

### B. Cell-Based Functional Assay of Muscarinic Acetylcholine Receptor Activity

For high throughput measurement of agonist-evoked increases in intracellular calcium, CHO-K1 cells stably expressing muscarinic receptors were plated in growth medium lacking G418 and hygromycin at 15,000 cells/20 μL/well in Greiner 384-well black-walled, tissue culture (TC)-treated, clear-bottom plates (VWR). Cells were incubated overnight at 37° C. and 5% CO₂. The next day, cells were washed using an ELX 405 (BioTek) with assay buffer; the final volume was then aspirated to 20 μL. Next, 20 μL of a 2.3 M stock of Fluo-4/acetoxymethyl ester (Invitrogen, Carlsbad, CA), prepared as a 2.3 mM stock in DMSO and mixed in a 1:1 ratio with 10% (w/v) Pluronic F-127 and diluted in assay buffer, was added to the wells and the cell plates were incubated for 50 min at 37° C. and 5% C02. Dye was removed by washing with the ELX 405 and the final volume was aspirated to 20 μL. Compound master plates were formatted in an 11 point concentration-response curve (CRC) format (1:3 dilutions) in 100% DMSO with a starting concentration of 10 mM using a BRAVO liquid handler (Agilent). Test compound CRCs were then transferred to daughter plates (240 nL) using the Echo acoustic plate reformatter (Labcyte, Sunnyvale, CA) and then diluted into assay buffer (40 μL) to a 2× stock using a Thermo Fisher Combi (Thermo Fisher Scientific, Waltham, MA).

Calcium flux was measured using the Functional Drug Screening System (FDSS) 6000 or 7000 (Hamamatsu Corporation, Tokyo, Japan) as an increase in the fluorescent static ratio. Compounds were applied to cells (20 μL, 2×) using the automated system of the FDSS at 2-4 seconds into the protocol and the data were collected at 1 Hz. At 144 seconds, 10 μL of an EC₂₀ concentration of the muscarinic receptor agonist acetylcholine was added (5×), followed by the addition of 12 μL of an EC₈₀ concentration of acetylcholine at the 230 second time point (5×). Agonist activity was analyzed as a concentration-dependent increase in calcium mobilization upon compound addition. Positive allosteric modulator activity was analyzed as a concentration-dependent increase in the EC₂₀ acetylcholine response. Antagonist activity was analyzed as a concentration-dependent decrease in the EC₈₀ acetylcholine response. Concentration-response curves were generated using a four-parameter logistical equation in XLFit curve fitting software (IDBS, Bridgewater, NJ) for Excel (Microsoft, Redmond, WA) or Prism (GraphPad Software, Inc., San Diego, CA).

The above described assay was also operated in a second mode where an appropriate fixed concentration of the present compounds were added to the cells after establishment of a fluorescence baseline for about 3 seconds, and the response in cells was measured. 140 s later, the appropriate concentration of agonist was added and the calcium response (maximum-local minima response) was measured. The EC₅₀ values for the agonist in the presence of test compound were determined by nonlinear curve fitting. A decrease in the EC₅₀ value of the agonist with increasing concentrations of the present compounds (a leftward shift of the agonist concentration-response curve) is an indication of the degree of muscarinic positive allosteric modulation at a given concentration of the present compound. An increase in the EC₅₀ value of the agonist with increasing concentrations of the present compounds (a rightward shift of the agonist concentration response curve) is an indication of the degree of muscarinic antagonism at a given concentration of the present compound. The second mode also indicates whether the present compounds also affect the maximum response of the muscarinic receptor to agonists.

C. Activity of Compounds in a mAChR M₄ Cell-Based Assay

Compounds were synthesized as described above. Activity (EC₅₀ and Eₘₐₓ) was determined in the mAChR M₄ cell-based functional assay as described above and the data are shown in Table 2. The compound number corresponds to the compound numbers used in Table 1.

TABLE 2

| | Human | |
|---|---|---|
| | M₄ EC₅₀ | Emax |
| No. | (nM) | (%)* |
| 1 | 286 | 86 |
| 2 | 457 | 85 |
| 3 | 1,740 | 87 |
| 4 | 1,270 | 55 |
| 5 | 2,400 | 63 |
| 6 | 223 | 77 |
| 7 | 90.5 | 94 |
| 8 | 1,370 | 83 |
| 9 | 785 | 76 |
| 10 | 240 | 56 |
| 11 | 393 | 70 |
| 12 | 273 | 67 |
| 13 | 303 | 64 |
| 14 | 455 | 70 |
| 15 | 421 | 32 |
| 16 | 378 | 61 |
| 17 | 560 | 33 |
| 18 | 192 | 52 |
| 19 | 349 | 48 |
| 20 | 890 | 62 |
| 21 | 558 | 39 |
| 22 | 132 | 43 |
| 23 | 926 | 63 |
| 24 | 948 | 46 |
| 25 | 1,910 | 45 |
| 26 | 1,040 | 43 |
| 27 | 966 | 29 |
| 28 | 599 | 35 |
| 29 | 783 | 65 |
| 30 | 790 | 86 |
| 31 | 3,160 | 76 |
| 32 | 219 | 74 |
| 33 | 197 | 66 |
| 34 | 314 | 77 |
| 35 | >10,000 | 62 |
| 36 | 698 | 66 |
| 37 | 438 | 57 |
| 38 | 226 | 71 |
| 39 | 239 | 73 |
| 40 | 910 | 51 |
| 41 | 483 | 46 |
| 42 | 483 | 80 |
| 43 | 1,160 | 71 |
| 44 | 1,880 | 40 |
| 45 | 1,190 | 97 |
| 46 | 970 | 34 |
| 47 | 136 | 81 |
| 48 | 1,810 | 82 |
| 49 | 868 | 60 |
| 50 | 1,790 | 68 |
| 51 | 778 | 61 |
| 52 | 305 | 64 |
| 53 | 479 | 70 |
| 54 | 391 | 70 |
| 55 | 195 | 76 |
| 56 | 364 | 53 |
| 57 | 10,000 | 63 |
| 58 | 10,000 | 51 |
| 59 | 6,910 | 68 |
| 60 | 4,720 | 61 |
| 61 | 1,630 | 67 |
| 62 | 760 | 78 |
| 63 | 2,710 | 68 |
| 64 | 10,000 | 59 |
| 65 | 10,000 | 52 |
| 66 | 10,000 | 47 |
| 67 | 1944 | 55 |
| 68 | 3140 | 43 |
| 69 | 2380 | 80 |
| 70 | 2930 | 80 |
| 71 | 3380 | 102 |
| 72 | 233 | 78 |
| 73 | 940 | 50 |
| 74 | 368 | 87 |
| 75 | 666 | 58 |
| 76 | 1490 | 76 |
| 77 | 198 | 89 |
| 78 | 324 | 65 |
| 79 | 232 | 82 |
| 80 | 300 | 72 |
| 81 | 539 | 78 |
| 82 | 157 | 82 |
| 83 | 1,540 | 77 |
| 84 | 2,240 | 71 |
| 85 | 361 | 86 |
| 86 | 654 | 84 |
| 87 | 815 | 82 |
| 88 | 731 | 86 |
| 89 | 716 | 80 |
| 90 | 7,600 | 72 |
| 91 | 5,570 | 67 |
| 92 | 218 | 82 |
| 93 | 123 | 95 |
| 94 | 96.9 | 91 |
| 95 | 84.1 | 98 |
| 96 | 302 | 95 |
| 97 | 680 | 81 |
| 98 | 407 | 79 |
| 99 | 740 | 57 |
| 100 | 188 | 80 |
| 101 | 339 | 91 |
| 102 | 362 | 86 |
| 103 | 158 | 95 |
| 104 | 216 | 99 |
| 105 | 263 | 98 |
| 106 | 1140 | 33 |
| 107 | 142 | 98 |
| 108 | 330 | 90 |
| 109 | 167 | 90 |
| 110 | 167 | 95 |
| 111 | 345 | 93 |
| 112 | 465 | 76 |
| 113 | 1,450 | 98 |
| 114 | 969 | 100 |
| 115 | 995 | 92 |
| 116 | 336 | 95 |
| 117 | 3650 | 55 |
| 118 | 2,820 | 92 |
| 119 | 191 | 82 |
| 120 | 2,680 | 64 |
| 121 | 544 | 70 |
| 122 | 10,000 | 55 |
| 123 | 10,000 | 82 |
| 124 | 2,170 | 78 |
| 125 | 856 | 78 |
| 126 | 9,080 | 63 |
| 127 | 2,620 | 67 |
| 128 | 1,150 | 74 |
| 129 | 3,340 | 61 |
| 130 | 507 | 82 |
| 131 | 876 | 57 |
| 132 | 234 | 96 |
| 133 | 158 | 100 |
| 134 | 1640 | 69 |
| 135 | 1800 | 84 |
| 136 | 1880 | 88 |
| 137 | 3380 | 91 |
| 138 | 1110 | 96 |
| 139 | 1360 | 70 |
| 140 | 1030 | 62 |
| 141 | 1420 | 56 |
| 142 | 10,000 | 50 |
| 143 | 5830 | 53 |
| 144 | 6090 | 61 |
| 145 | 10,000 | 50 |
| 146 | 10,000 | 56 |
| 147 | 10,000 | 52 |
| 148 | 3540 | 67 |
| 149 | 10,000 | 56 |
| 150 | 4680 | 61 |
| 151 | 3250 | 67 |
| 152 | 2120 | 64 |
| 153 | 3170 | 68 |
| 154 | 6560 | 47 |
| 155 | 3910 | 63 |
| 156 | 1150 | 84 |
| 157 | 3480 | 57 |
| 158 | 3640 | 68 |
| 159 | 3130 | 69 |
| 160 | 10,000 | 51 |
| 161 | 10,000 | 58 |
| 162 | 3350 | 65 |
| 163 | 2660 | 65 |
| 164 | 10,000 | 54 |
| 165 | 145 | 111 |
| 166 | 45.1 | 106 |
| 167 | 265 | 91 |
| 168 | 420 | 91 |
| 169 | 111 | 100 |
| 170 | 110 | 103 |
| 171 | 265 | 87 |
| 172 | 78.5 | 93 |
| 173 | 77.3 | 95 |
| 174 | 75.8 | 98 |
| 175 | 145 | 89 |
| 176 | 188 | 89 |
| 177 | 101 | 91 |
| 178 | 262 | 87 |
| 179 | 100 | 98 |
| 180 | 96.9 | 94 |
| 181 | 141 | 90 |
| 182 | 439 | 75 |
| 183 | 164 | 91 |
| 184 | 106 | 111 |
| 185 | 211 | 101 |
| 186 | 318 | 97 |
| 187 | 138 | 108 |
| 188 | 244 | 106 |
| 189 | 199 | 93 |
| 190 | 136 | 105 |
| 191 | 187 | 98 |
| 192 | 169 | 90 |
| 193 | 605 | 82 |
| 194 | 211 | 93 |
| 195 | 611 | 80 |
| 196 | 1170 | 70 |
| 197 | 63.0 | 92 |
| 198 | 137 | 92 |
| 199 | 140 | 81 |
| 200 | 128 | 96 |
| 201 | 176 | 77 |
| 202 | 921 | 78 |
| 203 | 647 | 86 |
| 204 | 328 | 90 |
| 205 | 1340 | 67 |
| 206 | 534 | 77 |
| 207 | 370 | 86 |
| 208 | 304 | 76 |
| 209 | 170 | 95 |
| 210 | 128 | 96 |
| 211 | 123 | 99 |
| 212 | 65 | 98 |
| 213 | 1890 | 94 |
| 214 | 709 | 103 |
| 215 | 137 | 93 |
| 216 | 864 | 94 |
| 217 | 539 | 96 |
| 218 | 759 | 83 |
| 219 | 10,000 | 37 |
| 220 | 518 | 82 |
| 221 | 10,000 | 25 |
| 222 | 333 | 91 |
| 223 | 3292 | 56 |
| 224 | 214 | 82 |
| 225 | 57 | 87 |
| 226 | 346 | 89 |
| 227 | 266 | 67 |
| 228 | 890 | 66 |
| 229 | 332 | 59 |
| 230 | 279 | 65 |
| 231 | 668 | 63 |
| 232 | 263 | 61 |
| 233 | 366 | 80 |
| 234 | 354 | 63 |
| 235 | 350 | 87 |
| 236 | 10,000 | 29 |
| 237 | 2958 | 73 |
| 238 | 819 | 36 |
| 239 | 911 | 58 |
| 240 | 154 | 61 |
| 241 | 111 | 81 |
| 242 | 1874 | 83 |
| 243 | 25 | 102 |
| 244 | 25 | 99 |
| 245 | 641 | 56 |
| 246 | 8392 | 19 |
| 247 | 764 | 91 |
| 248 | 300 | 88 |
| 249 | 1438 | 82 |
| 250 | 616 | 66 |
| 251 | 197 | 93 |
| 252 | 1030 | 75 |
| 253 | 200 | 94 |
| 254 | 70 | 95 |
| 255 | 2095 | 88 |
| 256 | 663 | 85 |
| 257 | 8310 | 39 |
| 258 | 69 | 79 |
| 259 | 217 | 91 |
| 260 | 154 | 87 |
| 261 | 358 | 72 |
| 262 | 275 | 77 |
| 263 | 145 | 90 |
| 264 | 1625 | 66 |
| 265 | 1475 | 88 |
| 266 | 4566 | 38 |
| 267 | 1027 | 87 |
| 268 | 4188 | 68 |
| 269 | 1079 | 89 |
| 270 | 896 | 70 |
| 271 | 214 | 78 |
| 272 | 466 | 78 |
| 273 | 639 | 92 |
| 274 | 735 | 87 |
| 275 | 981 | 86 |
| 276 | 377 | 100 |
| 277 | 92 | 98 |
| 278 | 178 | 95 |
| 279 | 191 | 103 |
| 280 | 261 | 105 |
| 281 | 3287 | 58 |
| 282 | 302 | 62 |
| 283 | 368 | 56 |
| 284 | 3260 | 35 |
| 285 | 1224 | 30 |
| 286 | 504 | 33 |
| 287 | 584 | 54 |
| 288 | 1216 | 51 |
| 289 | 918 | 51 |
| 290 | 991 | 42 |
| 291 | 88 | 96 |
| 292 | 163 | 86 |
| 293 | 354 | 85 |
| 294 | 292 | 83 |
| 295 | 485 | 95 |
| 296 | 191 | 101 |
| 297 | 267 | 83 |
| 298 | 530 | 93 |
| 299 | 178 | 64 |
| 300 | 246 | 71 |
| 301 | 265 | 57 |
| 302 | 1714 | 48 |
| 303 | 1604 | 48 |
| 304 | 676 | 93 |
| 305 | 537 | 105 |
| 306 | 23 | 109 |
| 307 | 162 | 100 |
| 308 | 405 | 94 |
| 309 | 770 | 96 |
| 310 | 600 | 90 |
| 311 | 239 | 94 |
| 312 | 458 | 50 |
| 313 | 605 | 42 |
| 314 | 26 | 94 |
| 315 | 961 | 47 |
| 316 | 689 | 57 |
| 317 | 98 | 85 |
| 318 | 470 | 74 |
| 319 | 144 | 76 |
| 320 | 2679 | 54 |
| 321 | 356 | 73 |
| 322 | 1042 | 86 |
| 323 | 764 | 86 |
| 324 | 1069 | 70 |
| 325 | 2949 | 51 |
| 326 | 611 | 68 |
| 327 | 1908 | 68 |
| 328 | 191 | 64 |
| 329 | 554 | 66 |
| 330 | 71 | 85 |
| 331 | 43 | 86 |
| 332 | 824 | 83 |
| 333 | 817 | 72 |
| 334 | 582 | 60 |
| 335 | 386 | 41 |
| 336 | 258 | 49 |
| 337 | 270 | 71 |
| 338 | 315 | 91 |
| 339 | 277 | 90 |
| 340 | 7551 | 81 |
| 341 | 1493 | 49 |
| 342 | 64 | 86 |
| 343 | 226 | 97 |
| 344 | 380 | 88 |
| 345 | 341 | 86 |
| 346 | 1298 | 88 |
| 347 | 992 | 82 |
| 348 | 331 | 70 |
| 349 | 1123 | 72 |
| 350 | 547 | 84 |
| 351 | 160 | 88 |
| 352 | 233 | 64 |
| 353 | 126 | 73 |
| 354 | 271 | 64 |
| 355 | 297 | 81 |
| 356 | 276 | 83 |
| 357 | 127 | 84 |
| 358 | 320 | 82 |
| 359 | 472 | 120 |
| 360 | 88 | 129 |
| 361 | 786 | 115 |
| 362 | 76 | 96 |
| 363 | 148 | 90 |
| 364 | 321 | 98 |
| 365 | 358 | 89 |
| 366 | 305 | 96 |
| 367 | 209 | 89 |
| 368 | 1704 | 94 |
| 369 | 424 | 63 |
| 370 | 81 | 82 |
| 371 | 4346 | 75 |
| 372 | 5803 | 55 |
| 373 | 1738 | 64 |
| 374 | 248 | 96 |
| 375 | 1320 | 85 |
| 376 | 1384 | 90 |
| 377 | 760 | 76 |
| 378 | 4305 | 62 |
| 379 | 3596 | 47 |
| 380 | 2152 | 63 |
| 381 | 384 | 98 |
| 382 | 74 | 98 |
| 383 | 171 | 97 |
| 384 | 185 | 100 |
| 385 | 40 | 103 |
| 386 | 76 | 103 |
| 387 | 861 | 71 |
| 388 | 1250 | 83 |
| 389 | 942 | 92 |
| 390 | 433 | 78 |
| 391 | 53 | 89 |
| 392 | 458 | 85 |
| 393 | 111 | 88 |
| 394 | 1079 | 86 |
| 395 | 389 | 90 |
| 396 | 65 | 87 |
| 397 | 441 | 72 |
| 398 | 102 | 74 |
| 399 | 219 | 73 |
| 400 | 1052 | 56 |
| 401 | 522 | 76 |
| 402 | 150 | 91 |
| 403 | 84 | 91 |
| 404 | 901 | 72 |
| 405 | 415 | 83 |
| 406 | 326 | 96 |
| 407 | 611 | 91 |
| 408 | 685 | 65 |
| 409 | 1104 | 96 |
| 410 | 3804 | 89 |
| 411 | 2250 | 47 |
| 412 | 306 | 62 |
| 413 | 257 | 102 |
| 414 | 739 | 65 |
| 415 | 94 | 72 |
| 416 | 116 | 115 |
| 417 | 24 | 110 |
| 418 | 516 | 80 |
| 419 | 284 | 87 |
| 420 | 10,000 | 43 |
| 421 | 336 | 89 |
| 422 | 523 | 94 |
| 423 | 229 | 76 |
| 424 | 1434 | 86 |
| 425 | 621 | 61 |
| 426 | 577 | 95 |
| 427 | 2670 | 63 |
| 428 | 2345 | 59 |
| 429 | 2516 | 57 |
| 430 | 6532 | 42 |
| 431 | 10,000 | 47 |
| 432 | 6796 | 48 |
| 433 | 897 | 74 |
| 434 | 958 | 102 |
| 435 | 914 | 61 |
| 436 | 69 | 98 |
| 437 | 692 | 47 |
| 438 | 517 | 88 |
| 439 | 138 | 108 |
| 440 | 86 | 107 |
| 441 | 143 | 88 |
| 442 | 424 | 96 |
| 443 | 157 | 107 |
| 444 | 196 | 100 |
| 445 | 170 | 100 |
| 446 | 1144 | 87 |
| 447 | 1644 | 97 |
| 448 | 537 | 102 |
| 449 | 84 | 105 |
| 450 | 215 | 71 |
| 451 | 150 | 92 |
| 452 | 1793 | 89 |
| 453 | 828 | 92 |
| 454 | 3905 | 60 |
| 455 | 581 | 93 |
| 456 | 888 | 75 |
| 457 | 576 | 85 |
| 458 | 562 | 84 |
| 459 | 1186 | 71 |
| 460 | 1835 | 71 |
| 461 | 4104 | 76 |
| 462 | 714 | 72 |
| 463 | 422 | 101 |
| 464 | 36 | 101 |
| 465 | 44 | 101 |
| 466 | 111 | 98 |
| 467 | 129 | 98 |
| 468 | 119 | 108 |
| 469 | 114 | 98 |
| 470 | 278 | 102 |
| 471 | 251 | 91 |
| 472 | 71 | 99 |
| 473 | 324 | 87 |
| 474 | 206 | 89 |
| 475 | 1742 | 64 |
| 476 | 752 | 67 |
| 477 | 2471 | 80 |
| 478 | 379 | 50 |
| 479 | 886 | 57 |
| 480 | 1394 | 76 |
| 481 | 1057 | 58 |
| 482 | 468 | 78 |
| 483 | 313 | 84 |
| 484 | 176 | 88 |
| 485 | 247 | 79 |
| 486 | 286 | 97 |
| 487 | 2875 | 38 |
| 488 | 217 | 89 |
| 489 | 5 | 95 |
| 490 | 16 | 100 |
| 491 | 15 | 91 |
| 492 | 17 | 110 |
| 493 | 37 | 112 |
| 494 | 36 | 106 |
| 495 | 43 | 102 |
| 496 | 37 | 102 |
| 497 | 64 | 101 |
| 498 | 3169 | 85 |
| 499 | 2672 | 88 |
| 500 | 1790 | 92 |
| 501 | 127 | 109 |
| 502 | 3533 | 105 |
| 503 | 4080 | 103 |
| 504 | 3875 | 101 |
| 505 | 21 | 112 |
| 506 | 2806 | 84 |
| 507 | 43 | 81 |
| 508 | 13 | 82 |
| 509 | 12 | 104 |
| 510 | 855 | 81 |
| 511 | 3161 | 75 |
| 512 | 19 | 95 |
| 513 | 51 | 90 |
| 514 | 635 | 103 |
| 515 | 197 | 107 |
| 516 | 31 | 102 |
| 517 | 12 | 76 |
| 518 | 32 | 102 |
| 519 | 14 | 80 |
| 520 | 43 | 104 |
| 521 | 98 | 99 |
| 522 | 168 | 109 |
| 523 | 74 | 109 |
| 524 | 18 | 93 |
| 525 | 11 | 82 |
| 526 | 17 | 116 |
| 527 | 16 | 129 |
| 528 | 53 | 102 |
| 529 | 570 | 68 |
| 530 | 3034 | 81 |
| 531 | 241 | 83 |
| 532 | 78 | 90 |
| 533 | 107 | 88 |
| 534 | 322 | 88 |
| 535 | 85 | 84 |
| 536 | 1151 | 86 |
| 537 | 131 | 96 |
| 538 | 54 | 103 |
| 539 | 51 | 101 |
| 540 | 268 | 100 |
| 541 | 61 | 101 |
| 542 | 64 | 113 |
| 543 | 47 | 114 |
| 544 | 45 | 105 |
| 545 | 46 | 125 |
| 546 | 16 | 116 |
| 547 | 40 | 122 |
| 548 | 25 | 115 |
| 549 | 56 | 120 |
| 550 | 86 | 111 |
| 551 | 44 | 111 |
| 552 | 22 | 76 |
| 553 | 21 | 80 |
| 554 | 16 | 78 |
| 555 | 48 | 83 |
| 556 | 1457 | 87 |
| 557 | 166 | 91 |
| 558 | 101 | 90 |
| 559 | 64 | 73 |
| 560 | 64 | 75 |
| 561 | 169 | 82 |
| 562 | 91 | 74 |
| 563 | 74 | 69 |
| 564 | 86 | 72 |
| 565 | 128 | 72 |
| 566 | 41 | 73 |
| 567 | 70 | 75 |
| 568 | 161 | 75 |
| 569 | 200 | 78 |
| 570 | 340 | 84 |
| 571 | 213 | 85 |
| 572 | 51 | 87 |
| 573 | 100 | 89 |
| 574 | 193 | 81 |
| 575 | 88 | 75 |
| 576 | 41 | 71 |
| 577 | 109 | 70 |
| 578 | 2438 | 105 |
| 579 | 50 | 111 |
| 580 | 108 | 75 |
| 581 | 60 | 96 |
| 582 | 1912 | 76 |
| 583 | 121 | 91 |
| 584 | 533 | 91 |
| 585 | 482 | 96 |
| 586 | 176 | 99 |
| 587 | 6543 | 69 |
| 588 | 322 | 93 |
| 589 | 173 | 99 |
| 590 | 41 | 94 |
| 591 | 1989 | 56 |
| 592 | 292 | 38 |
| 593 | 876 | 70 |
| 594 | 132 | 84 |
| 595 | 4466 | 38 |
| 596 | 1879 | 54 |
| 597 | 2870 | 69 |
| 598 | 25 | 98 |
| 599 | 7 | 96 |
| 600 | 26 | 92 |
| 601 | 7 | 95 |
| 602 | 35 | 92 |
| 603 | 28 | 97 |
| 604 | 158 | 83 |
| 605 | 1444 | 106 |
| 606 | 10 | 90 |
| 607 | 906 | 70 |
| 608 | 4 | 83 |
| 609 | 753 | 56 |
| 610 | 59 | 70 |
| 611 | 8 | 86 |
| 612 | 24 | 91 |
| 613 | 3 | 97 |
| 614 | 12 | 92 |
| 615 | 11 | 89 |
| 616 | 4 | 95 |
| 617 | 2455 | 88 |
| 618 | 391 | 74 |
| 619 | 1021 | 86 |
| 620 | 72 | 96 |
| 621 | 318 | 85 |

*% ACh maximum at 30 μM.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A compound of formula (I), or a pharmaceutically acceptable salt thereof, 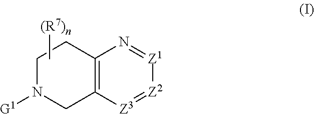
wherein: 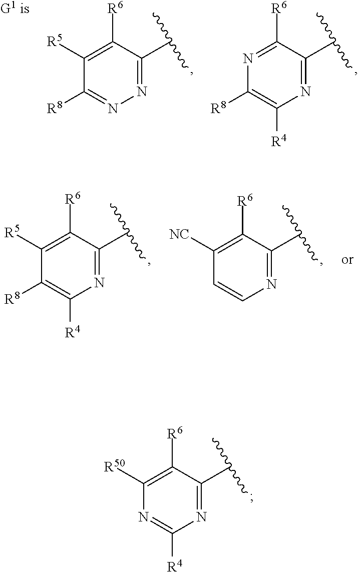
Z¹ is CR¹;
Z² is CR²;
Z³ is CR³;
R¹ is hydrogen;
R² is G², —NR^{c}, halogen, cyano, NO₂, C₁₋₆alkyl, C₁₋₆haloalkyl, —OR^{b}, —SR^{b}, —OC(O)R^{b}, —NR^{b}C(O)R^{c}, —NR^{b}SO₂R^{a}, —C(O)OR^{b}, —C(O)NR^{b}R^{c}, —SO₂NR^{b}R^{c}, —C(O)R^{b}, —S(O)R^{a}, —SO₂R^{a}, —C₁₋₆alkylene-OH, —C₁₋₆fluoroalkylene-OH, or —C₁₋₃alkylene-G², or hydrogen;
R³ is hydrogen;
R^{a}, at each occurrence, is independently C₁₋₆alkyl, C₁₋₆haloalkyl, G², or —C₁₋₃alkylene-G²;
R^{b} and R^{c}, at each occurrence, are independently hydrogen, C₁₋₆alkyl, C₁₋₆haloalkyl, G², —C1-3alkylene-G², —C₂₋₄alkylene-O—C₁₋₄alkyl, or —C₂₋₄alkylene-O—G²;
G², at each occurrence, is independently a a 4- to 12-membered heterocyclyl, a 5- to 12-membered heteroaryl, 6- to 12-membered aryl, or a 3- to 12-membered carbocyclyl, wherein G² is optionally substituted with 1-5 substituents independently selected from the group consisting of halogen, cyano, C₁₋₆alkyl, C₁₋₆haloalkyl, oxo, —OR^{x}, —N(R^{x})₂, —C₁₋₆alkylene-OR^{x}, —C₁₋₆alkylene-N(R^{x})₂, G^{2a}, and —C₁₋₃alkylene-G^{2a};
R^{x}, at each occurrence, is independently hydrogen, C₁₋₄alkyl, C₁₋₄haloalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄haloalkyl;
R⁴ is hydrogen, halogen, cyano, C₁₋₆alkyl, C₁₋₆haloalkyl, —C₁₋₆alkylene-OR^{4a}, —OR^{4a}, —O—C₂₋₆alkylene-OR^{4a}, —N(R^{4a})₂, —N(R^{4a})—C₂₋₆alkylene-OR^{4a}, G³, —O—G³, —N(R^{4a})—G³, —C₁₋₃alkylene-G³, —O—C₁₋₃alkylene-G³, or —N(R^{4a})—C₁₋₃alkylene-G³;
R^{4a}, at each occurrence, is independently hydrogen, C₁₋₆alkyl, or C₁₋₆haloalkyl;
R⁵ and R⁶ are each independently hydrogen, halogen, cyano, C₁₋₆alkyl, C₁₋₆haloalkyl, OH, —OC₁₋₆alkyl, —OC₁₋₆haloalkyl, G³, —O—G³, —C₁₋₃alkylene-G³, or —O—C₁₋₃alkylene-G³;
R⁵⁰ is hydrogen, halogen, cyano, C₁₋₆alkyl, C₁₋₆haloalkyl, OH, —OC₁₋₆alkyl, —OC₁₋₆haloalkyl, G³⁰, —O—G³, —C(O)—N(R^{50a})₂—C₁₋₃alkylene-G³, or —O—C₁₋₃alkylene-G³;
R^{50a}, at each occurrence, is independently hydrogen, C₁₋₄alkyl, G³, or —C₁₋₃alkylene-G³, or two
R^{50a}, together with the nitrogen to which they attach, form a 4- to 8-membered heterocyclyl, the heterocyclyl optionally containing a second heteroatom that is O, N, or S and being optionally substituted with 1-4 C₁₋₄alkyl;
wherein, alternatively, R⁵⁰ and R⁶, together with the atom to which each attaches, form a 5- to 7-membered non-aromatic carbocyclic or heterocyclic ring, the heterocyclic ring containing one heteroatom that is O, N, or S and the carbocyclic or heterocyclic ring being optionally substituted with 1-4 C₁₋₄alkyl;
G³, at each occurrence, is independently a 6- to 12-membered aryl, a 5- to 12-membered heteroaryl, a 4- to 12-membered heterocyclyl, or a 3- to 12-membered carbocyclyl, wherein G³ is optionally substituted with 1-5 substituents independently selected from the group consisting of halogen, cyano, C₁₋₄alkyl, C₁₋₄haloalkyl, oxo, OH, —OC₁₋₄alkyl, —OC₁₋₄haloalkyl, G^{3a}, and —C₁₋₃alkylene-G^{3a};
G³⁰ is a 6- to 12-membered aryl, a 5- to 12-membered heteroaryl other than indazol-3-yl, a 4- to 12-membered heterocyclyl, or a 3- to 12-membered carbocyclyl, wherein G³⁰ is optionally substituted with 1-5 substituents independently selected from the group consisting of halogen, cyano, C₁₋₄alkyl, C₁₋₄haloalkyl, oxo, OH, —OC₁₋₄alkyl, —OC₁₋₄haloalkyl, G^{3a}, and —C₁₋₃alkylene-G^{3a};
R⁷, at each occurrence, is independently halogen, C₁₋₄alkyl, C₁₋₄haloalkyl, C₃₋₇cycloalkyl, OH, —OC₁₋₄alkyl, or —OC₁₋₄haloalkyl, wherein the cycloalkyl is optionally substituted with 1-4 substituents independently selected from the group consisting of halogen, C₁₋₄alkyl, and C₁₋₄haloalkyl;
R⁸ is cyano, —C(O)R^{Bb}, halogen, C₁₋₆alkyl, C₁₋₆haloalkyl, —OR^{8b}, —NR^{8b}R^{8c}, —SR^{8b}, —OC(O)R^{8b}—NR^{8b}C(O)R^{8c}—NR^{8b}SO₂R^{8a}, —C(O)OR^{8b}, —C(O)NR^{8b}R^{8c}, —SO₂NR^{8b}R^{8c}, —S(O)R^{8a}, —SO₂R^{8a}, G⁴, or —C₁₋₃alkylene-G⁴;
R^{8a}, at each occurrence, is independently C₁₋₆alkyl, C₁₋₆haloalkyl, G⁴, or —C₁₋₃alkylene-G⁴;
R^{8b} and R^{8c}, at each occurrence, are independently G⁴, hydrogen, C₁₋₆alkyl, C₁₋₆haloalkyl, —C₁₋₆alkylene-OH, or —C₁₋₃alkylene-G⁴;
wherein, alternatively, when R⁸ is —C(O)NR^{8b}R^{8c}, and R⁴ is —OR^{4a}, R^{8c} and R^{4a}, together with the atom to which each attaches, form an oxazepin-5-one ring;
G⁴, at each occurrence, is independently a 4- to 12-membered heterocyclyl, a 6- to 12-membered aryl, a 5- to 12-membered heteroaryl, or a 3- to 12-membered carbocyclyl optionally fused to a 6-membered arene or heteroarene that contains 1-2 nitrogen atoms, wherein G⁴ is optionally substituted with 1-5 substituents independently selected from the group consisting of halogen, cyano, C₁₋₄alkyl, C₁₋₄haloalkyl, —C₁₋₆alkylene-OH, oxo, —OR⁸⁰, —N(R⁸⁰)₂, —NR⁸⁰C(O)R⁸⁰, —NR⁸⁰SO₂R⁸⁰, —C(O)OR⁸⁰, —C(O)N(R⁸⁰)₂, —SO₂R⁸⁰, G^{4a}, and —C₁₋₃alkylene-G^{4a};
R⁸⁰, at each occurrence, is independently hydrogen, C₁₋₄alkyl, C₁₋₄haloalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄haloalkyl;
G^{2a}, G^{3a}, and G^{4a}, at each occurrence, are independently a phenyl, a 5- to 6-membered heteroaryl, a 4- to 8-membered heterocyclyl, or a 3- to 8-membered carbocyclyl, wherein G^{2a}, G^{3a}, and G^{4a}, at each occurrence, are independently optionally substituted with 1-5 substituents independently selected from the group consisting of halogen, cyano, C₁₋₄alkyl, C₁₋₄haloalkyl, —C₁₋₆alkylene-OH, oxo, OH, —OC₁₋₄alkyl, —OC₁₋₄haloalkyl, C₃₋₄cycloalkyl, and —C₁₋₃alkylene-C₃₋₄cycloalkyl;
and
n is 0, 1, 2, 3, or 4;
provided that the compound is not
6-(7,8-dihydro-1,6-naphthyridin-6(5H)-yl)-N-[2-(1-methyl-2-piperidinyl)ethyl]-3-pyridinecarboxamide;
6-(7,8-dihydro-1,6-naphthyridin-6(5H)-yl)-N-[(3-propyl-5-isoxazolyl)methyl]-3-pyridinecarboxamide; or
6-(6-methyl-2-(pyridin-3-yl)pyrimidin-4-yl)-5,6,7,8-tetrahydro-1,6-naphthyridine;
or a salt thereof.

2. The compound of claim 1, or a pharmaceutically acceptable salt thereof, wherein:
G¹ is 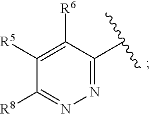
R⁵ is hydrogen or C₁₋₄alkyl; and
R⁶ is C₁₋₄alkyl.

3. The compound of claim 1, or a pharmaceutically acceptable salt thereof, wherein G¹ is 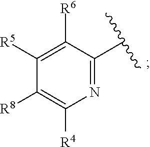
R⁵ is hydrogen; and
R⁶ is halogen, C₁₋₄alkyl, or C₁₋₄fluoroalkyl.

4. The compound of claim 3, or a pharmaceutically acceptable salt thereof, wherein R⁴ is hydrogen, —OC₁₋₄alkyl, or a 5- to 6-membered heteroaryl containing 1-3 heteroatoms that are independently O, N, or S, the heteroaryl being optionally substituted with 1-3 C₁₋₄alkyl.

5. The compound of claim 1, or a pharmaceutically acceptable salt thereof, wherein G¹ is 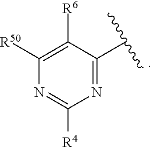
R⁴ is halogen, C₁₋₆alkyl, C₁₋₆haloalkyl, —C₁₋₆alkylene-OR^{4a}, —N(R^{4a})—C₂₋₆alkylene-OR^{4a}, G³, or —N(R^{4a})—G³.

6. The compound of claim 5, or a pharmaceutically acceptable salt thereof, wherein
R⁵⁰ is halogen, C₁₋₄alkyl, —OC₁₋₄alkyl, —C(O)—N(R^{50a})₂, or G³⁰; and
R⁶ is C₁₋₄alkyl.

7. The compound of claim 5, or a pharmaceutically acceptable salt thereof, wherein R⁵⁰ and R⁶, together with the atom to which each attaches, form a non-aromatic carbocyclic or heterocyclic ring, the heterocyclic ring containing one heteroatom that is O, N, or S and the carbocyclic or heterocyclic ring being optionally substituted with 1-4 C₁₋₄alkyl.

8. The compound of any of claims 1-7, or a pharmaceutically acceptable salt thereof, R² is G², —RR, halogen, NO₂, C₁₋₆alkyl, C₁₋₆haloalkyl, —OR^{b}, —C₁₋₆alkylene-OH, —C₁₋₆fluoroalkylene-OH, —C₁₋₃alkylene-G², or hydrogen.

9. The compound of claim 8, or a pharmaceutically acceptable salt thereof, wherein R² is G²; and
G² is:
a) a 4- to 10-membered heterocyclyl containing 1-3 heteroatoms independently selected from the group consisting of 0, N, and S;
b) a 5- to 6-membered or 9- to 10-membered heteroaryl containing 1-3 heteroatoms independently selected from the group consisting of O, N, and S;
c) C₃₋₆cycloalkyl; or
d) phenyl;
wherein G² is optionally substituted with 1-4 substituents independently selected from the group consisting of halogen, cyano, C₁₋₆alkyl, C₁₋₆haloalkyl, oxo, —OR^{x}, —N(R^{x})₂, —C₁₋₆alkylene-OR^{x}, —C₁₋₆alkylene-N(R^{x})₂, G^{2a}, and —C₁₋₃alkylene-G^{2a}; and
G^{2a} is C₃₋₆cycloalkyl, phenyl, a 5- to 6-membered heteroaryl containing 1-3 heteroatoms independently selected from the group consisting of O, N, and S, or a 4- to 8-membered heterocyclyl containing 1-3 heteroatoms independently selected from the group consisting of O, N, and S, G^{2a} being optionally substituted with 1-4 substituents independently selected from the group consisting of halogen, C₁₋₄alkyl, C₁₋₄fluoroalkyl, —C₁₋₆alkylene-OH, C₃₋₄cycloalkyl, and —C₁₋₃alkylene-C₃₋₄cycloalkyl.

10. The compound of claim 9, or a pharmaceutically acceptable salt thereof, wherein G² is

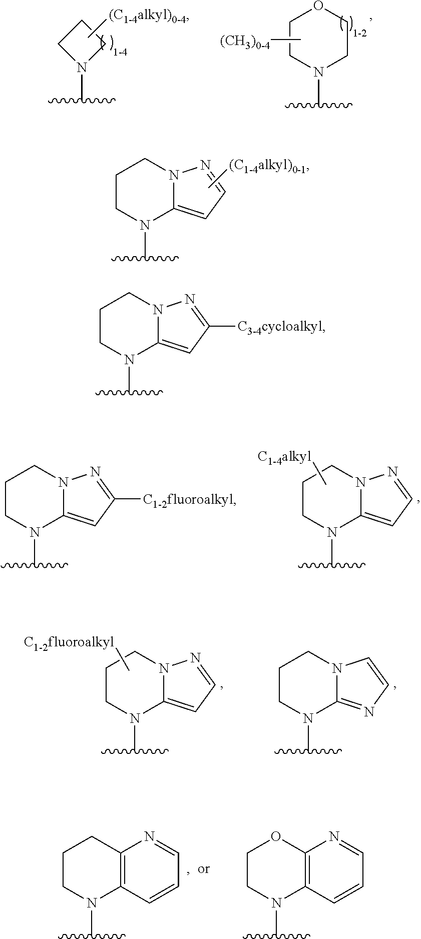

11. The compound of claim 9, or a pharmaceutically acceptable salt thereof, wherein G² is

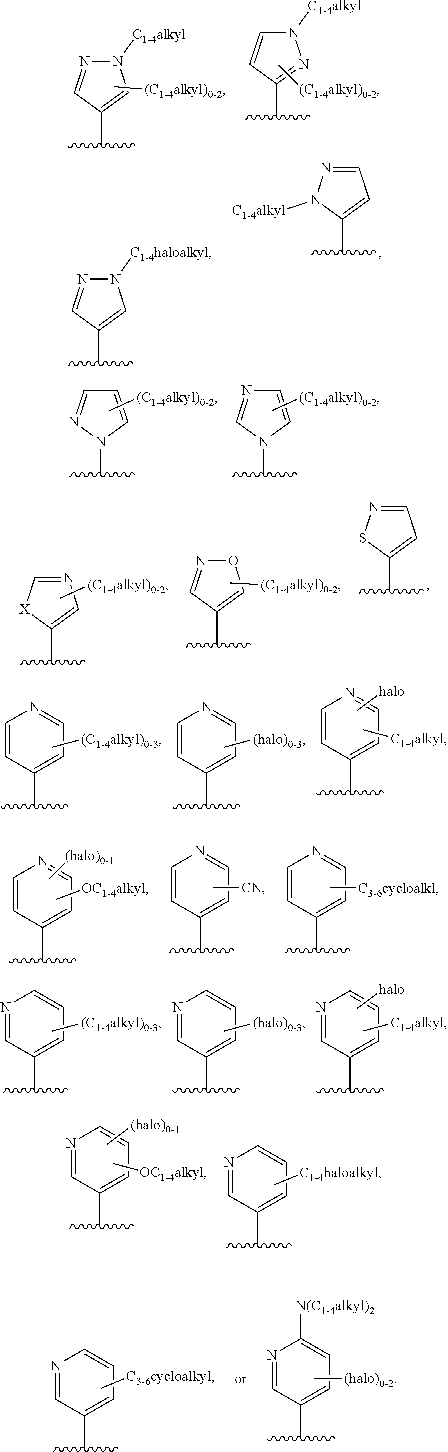

12. The compound of claim 8, or a pharmaceutically acceptable salt thereof, wherein
R² is —NR^{b}R^{c};
R^{b} is —G² or —C₁₋₃alkylene-G²; and
R^{c} is hydrogen, C₁₋₆alkyl, C₁₋₆haloalkyl, C₃₋₆cycloalkyl, or —C₁₋₃alkylene-C₃₋₆cycloalkyl.

13. The compound of claim 12, or a pharmaceutically acceptable salt thereof, wherein
R^{b} is —G²;
G² is a 5- to 6-membered heteroaryl containing 1-3 heteroatoms independently selected from the group consisting of O, N, and S, or G² is phenyl, wherein G² is optionally substituted with 1-4 substituents independently selected from the group consisting of halogen, cyano, C₁₋₆alkyl, C₁₋₆haloalkyl, —OR^{x}, —N(R^{x})₂, G^{2a}, and —C₁₋₃alkylene-G^{2a}; and
G^{2a} is C₃₋₆cycloalkyl.

14. The compound of claim 13, or a pharmaceutically acceptable salt thereof, wherein R² is

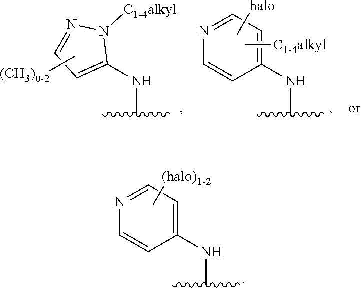

15. The compound of claim 12, or a pharmaceutically acceptable salt thereof, wherein R^{b} is —C₁₋₃alkylene-G²;
G² is phenyl, a 5- to 6-membered heteroaryl containing 1-3 heteroatoms independently selected from the group consisting of O, N, and S, or a 4- to 8-membered heterocyclyl containing 1-2 heteroatoms independently selected from the group consisting of O, N, and S, wherein G² is optionally substituted with 1-4 substituents independently selected from the group consisting of halogen, cyano, C₁₋₆alkyl, C₁₋₆haloalkyl, —OR^{x}, —N(R^{x})₂, G^{2a}, and —C₁₋₃alkylene-G^{2a}; and
G^{2a} is C₃₋₆cycloalkyl.

16. The compound of claim 8, or a pharmaceutically acceptable salt thereof, wherein
R² is —OR^{b};
R^{b} is G²; and
G² is phenyl, a phenyl fused to a 5- to 7-membered heterocycle containing 1-2 oxygen atoms, a 5- to 6-membered heteroaryl, or a 9- to 10-membered heteroaryl, each heteroaryl containing 1-3 heteroatoms independently selected from the group consisting of O, N, and S, G² being optionally substituted with 1-4 substituents independently selected from the group consisting of halogen, cyano, C₁₋₄alkyl, C₁₋₄haloalkyl, —OR^{x}, and —N(R^{x})₂.

17. The compound of claim 8, or a pharmaceutically acceptable salt thereof, wherein
R² is —OR^{b}; and
R^{b} is —C₂₋₄alkylene-O—C₁₋₄alkyl or —C₂₋₄alkylene-O—G².

18. The compound of claim 8, or a pharmaceutically acceptable salt thereof, wherein R² is —C₁₋₃alkylene-G².

19. The compound of any of claims 1-4 or 8-18, or a pharmaceutically acceptable salt thereof, wherein R⁸ is cyano, —C(O)OR^{8b}, —C(O)R^{8b}, or —C(O)NR^{8b}R^{8c}.

20. The compound of claim 19, or a pharmaceutically acceptable salt thereof, wherein R⁸ is cyano.

21. The compound of claim 19, or a pharmaceutically acceptable salt thereof, wherein
R⁸ is —C(O)R^{b};
R^{8b} is G⁴; and
G⁴ is a 4- to 10-membered heterocyclyl containing 1-3 heteroatoms independently selected from the group consisting of 0, N, and S; wherein G⁴ is optionally substituted with 1-5 substituents independently selected from the group consisting of halogen, cyano, C₁₋₄alkyl, C₁₋₄haloalkyl, —C₁₋₆alkylene-OH, oxo, —OR⁸⁰, —N(R⁸⁰)₂, —NR⁸⁰ C(O)R⁸⁰, —NR⁸⁰ SO₂R⁸⁰, —C(O)OR⁸⁰, —C(O)N(R⁸⁰)₂, —SO₂R⁸°, G^{4a}, and —C₁₋₃alkylene-G^{4a}.

22. The compound of claim 21, or a pharmaceutically acceptable salt thereof, wherein G⁴ is

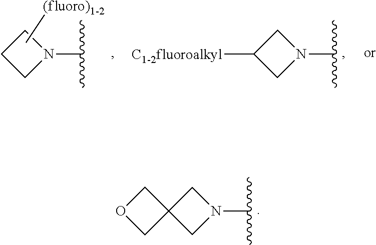

23. The compound of claim 19, or a pharmaceutically acceptable salt thereof, wherein R⁸ is —C(O)NR^{8b}R^{8c}.

24. The compound of claim 23, or a pharmaceutically acceptable salt thereof, wherein
R^{8b} is C₁₋₆alkyl, G⁴, or —C₁₋₃alkylene-G⁴; and
G⁴ is
a) a 5- to 6-membered or 9- to 10-membered heteroaryl containing 1-3 heteroatoms independently selected from the group consisting of 0, N, and S;
b) phenyl;
c) a phenyl fused to a 5- to 7-membered heterocycle containing 1-2 heteroatoms independently selected from the group consisting of 0, N, and S;
d) a 4- to 10-membered heterocyclyl containing 1-3 heteroatoms independently selected from the group consisting of 0, N, and S; or
e) a C₃₋₈cycloalkyl;
wherein G⁴ is optionally substituted with 1-5 substituents independently selected from the group consisting of halogen, cyano, C₁₋₄alkyl, C₁₋₄haloalkyl, —C₁₋₆alkylene-OH, oxo, —OR⁸⁰, —N(R⁸⁰)₂, —NR⁸⁰ C(O)R⁸⁰, —NR⁸⁰ SO₂R⁸⁰, —C(O)OR^{8c}, —C(O)N(R⁸⁰)₂, —SO₂R⁸⁰, G⁴a and —C₁₋₃alkylene-G4a.

25. The compound of claim 23, or a pharmaceutically acceptable salt thereof, wherein R⁴ is —OR^{4a}, and R^{8c} and R^{4a}, together with the atom to which each attaches, form an oxazepin-5-one ring.

26. The compound of any of claims 1-25, or a pharmaceutically acceptable salt thereof, wherein n is 0, 1, or 2; and R⁷ is C₁₋₄alkyl.

27. A pharmaceutical composition comprising the compound of any of claims 1-26 and a pharmaceutically acceptable carrier.

28. A method for treating a neurological and/or psychiatric disorder associated with muscarinic acetylcholine receptor dysfunction in a mammal, comprising administering to the mammal a therapeutically effective amount of the compound of any of claims 1-26, or pharmaceutically acceptable salt thereof, or the pharmaceutical composition of claim 27.
